(19) United States
(12) Patent Application Publication
(10) Pub. No.: US 2005/0215764 A1
(43) Pub. Date: Sep. 29, 2005

(54) Biological polymer with differently charged portions

(76) Inventors: Jack A. Tuszynski, Edmonton (CA); Kendrick Goss, Brighton, MA (US); Howard J. Greenwald, Rochester, NY (US)

Correspondence Address: HOWARD J. GREENWALD P.C., 349 W. COMMERCIAL STREET SUITE 2490, EAST ROCHESTER, NY, 14445-2408, US

(21) Appl. No.: 11/060,868

(22) Filed: Feb. 18, 2005

Related U.S. Application Data
(63) Continuation-in-part of application No. 10/923,615, filed on Aug. 20, 2004.
(63) Continuation-in-part of application No. 10/808,618, filed on Mar. 24, 2004.
(63) Continuation-in-part of application No. 10/867,517, filed on Jun. 14, 2004.
(63) Continuation-in-part of application No. 10/878,905, filed on Jun. 28, 2004.

(51) Int. Cl. C07K 14/47

(52) U.S. Cl. 530/358

(57) ABSTRACT

A biological polymer assembly with a biological polymer that contains at least 90 percent of tubulin and a positively charged segment. The positively charged segment has a molecular weight of at least about 5,000 Daltons, a bulk electrical conductivity of at least about 10⁻⁷ ohms⁻¹ meter⁻¹ Siemens, a concentration of elemental charges per cubic centimeter of from about 10¹² to about 10²⁵, and a length of at least about 2 nanometers.

### CROSS-REFERENCE TO RELATED PATENT APPLICATIONS

This application claims priority from United States provisional patent application U.S. Ser. No. 60/516,134, filed on Oct. 31, 2003, the entire disclosure of which is hereby incorporated by reference into this specification.

This application is a continuation-in-part of applicants' U.S. patent application Ser. No. 10/923,615, (filed on Aug. 20, 2004), Ser. No. 10/808,618 (filed on Mar. 24, 2004), of applicants' U.S. patent application Ser. No. 10/867,517 (filed on Jun. 14, 2004), and of applicants' U.S. patent application Ser. No. 10/878,905(filed on Jun. 28, 2004).

### FIELD OF THE INVENTION

A biological polymer tubulin-containing assembly that contains positively charged region that has a molecular weight of at least 5,000 Daltons, a bulk electrical conductivity of at least about 10⁻⁷ ohm⁻¹ meter⁻¹ Siemens, at least 10¹² positive charges per cubic centimeter, and a length of at least 2 namometers.

### BACKGROUND OF THE INVENTION

Conductive microtubules are known to those skilled in the art. As is disclosed in U.S. Pat. No. 6,452,564, the entire disclosure of which is hereby incorporated by reference into this specification, “ . . . these microtubules are preferably a system of biologically-derived, high-aspect ratio, rods or tubules of microscopic dimensions, and are made electrically conductive by electroless plating . . . .”

Preparation of the microtubules of U.S. Pat. No. 6,452,564 is described in the paragraph beginning at line 51 of column 4, wherein it is disclosed that: “The microtubules are based on research done a number of years ago, wherein researchers at the Naval Research Laboratories in Washington, D.C., discovered particles with the size and shape appropriate for percolation. These microtubules are biologically derived, hollow organic cylinders of half-micron diameter and lengths of tens to hundreds of microns. The cylinders are coated with metal to render them conductive by an electroless process. Once metallized, the microtubules can be dried to a powder and dispersed into polymer matrices at varying loading densities to form the composite. In a preferred embodiment, the microtubules are formed from diacetylenic lipid (1,2bis(tricosa-10,12-diynoyl)-sn-glycero-3-phosphocholine), or DC8,9PC. See, for example, A. N. Lagarkov and A. K. Sarychev, Phys. Rev. B 53, 6318 (1996) and F. Behroozi, M. Orman, R. Reese, W. Stockton, J. Calvert, F. Rachfold and P. Schoen, J. Appl. Phys. 68, 3688 (1990). The lipid is dissolved in alcohol at 50° C., water is added, and the temperature lowered to room temperature. The lipid self-assembles itself into microtubules and subsequently precipitates. The particles are rinsed and coated with a palladium catalyst and mixed with metal ions and reductants. In contact with the catalyst, the metal ions-are reduced to neutral metal on the surface of the microtubules and coat the structure with a conductive layer of metal of several tenths of a micron thickness. Several metal species are available for use in this process, but nickel and copper appear to be of greatest potential usefulness for the present invention.”

The microtubules of U.S. Pat. No. 6,452,564 have a substantially uniform conductivity over their entire surface, such conductivity being substantially equal to the conductivity of the metal used to coat the surface.

In some applications, such as, e.g., for semiconductor applications, it is desired to have microtubules with regions with distinct and separate electrical charges and/or charge polarities. It is an object of this invention to provide such a biological polymer.

### SUMMARY OF THE INVENTION

In accordance with this invention, there is provided a biological polymer tubulin-containing assembly that contains positively charged region that has a molecular weight of at least 5,000 Daltons, a bulk electrical conductivity of at least about 10⁻⁷ ohm⁻¹ meter⁻¹ Siemens, at least 10¹² positive charges per cubic centimeter, and a length of at least 2 namometers.

### BRIEF DESCRIPTION OF THE DRAWINGS

The invention will be described with referene to the specification and the enclosed drawings, in which like numerals refer to like elements, and wherein:

FIG. 1 is a schematic illustration of one preferred implantable assembly of the invention;

FIG. 2 is a schematic illustration of a flow meter that may be used in conjunction with the implantable assembly of claim 1;

FIG. 3 is a flow diagram of one preferred process of the invention;

FIG. 4 is a flow diagram of another preferred process of the invention;

FIG. 5 is a flow diagram of yet another preferred process of the invention;

FIG. 6 is a schematic of one preferred electrical circuit;

FIG. 7 is a flow diagram of a preferred process of the invention;

FIGS. 8A and 8B are schematic illustrations of one preferred process of Figure of the invention; 7;

FIG. 9 is a series of schematic illustrations of some of the preferred charged biological assemblies that may be made by the process of the invention;

FIG. 10 is a flow diagram illustrating a preferred process for the preparation of conductive DNA polymeric segments;

FIG. 11 is a schematic illustration of two complementary thiol-terminated oligonucleotides binding to each other;

FIG. 12 is a schematic illustration of two tubulin assemblies bound to oligonucleotide segments;

FIG. 13 is a schematic illustration of a microtubule assembly comprised of a conductive oligonucleotide segment;

FIG. 14 is a schematic illustration of microtubule assemblies bound electrostatically by oligonucleotide segments;

FIG. 15 is a schematic representation of the disassembly of microtubular polypeptides into component monomers;

FIG. 16 is a schematic representation of a microtubule and its associated electrical properties;

FIG. 17 is a schematic representation of microtubule with a coating and a notation representing its electrical properties;

FIGS. 18 and 19 each is a schematic representation of a microtubule and a notation describing its electrical properties;

FIG. 20 is a representation of a three-dimensional array of microtubules;

FIG. 21 is a schematic representation of an inductive assembly comprised of microtubules with associated proteins;

FIG. 22 is a schematic representation of an electrical switch comprised of recognition molecules;

FIG. 23 is a schematic representation of a circuit with multiple P and N sections made from biological material and notation describing its electrical properties;

FIG. 24 is a plan view of a biological switching device;

FIG. 25 is a cross sectional view of another biological switching device;

FIG. 26 is a time variant plan view of a biological switching device used as a sensor;

FIG. 27 is a cutaway view of a biological memory array;

FIG. 28 is a perspective view of a magnetic biological memory array element;

FIG. 29 is a plan view of the microelectrode and nanoelectrode structure;

FIG. 30 is a schematic illustration of a process for nanoelectrode fabrication;

FIG. 31 is a flow diagram of a process for influencing cellular processes;

FIG. 32 is a illustration of certain equations that may be ued in conjunction with the process depicted in FIG. 31;

FIG. 33 is a schematic of a device for interrogating cells;

FIGS. 34 and 35 illustrate a preferred process for repairing nerve damage; and

FIG. 36 is a flow diagram of a process for treating cells with electromagnetic energy.

### DESCRIPTION OF THE PREFERRED EMBODIMENTS

This specification will describe several different inventions, all of which relate to either tubulin compositions and/or microtubule compositons and/or the uses of such compositions and/or reagents that may be used in conjunction with such compositions.

In the first portion of this specification, applicants will discuss the preparation of a database of tubulin isotopes. In the second part of this specification, applicants will discuss certain preferred, magnetic compounds that, in one embodiment, target such tubulin isotypes and/or the microtubules they make up. In the third part of this specification, applicants will discuss a process for treating a biological organism in which the magnetic anti-mitotic compound may be used to both synchronize certain cells and immobilize the microtubules within such cells prior to the time such cells are subjected to mechanical vibrational energy. Thereafter, applicants will discuss other embodiments of the invention, including the preparation and use of a biological polymer that contains a region that has a molecular weight of at least 30,000 Daltons, a bulk electrical conductivity of at least about 10⁻⁷ ohm⁻¹ meter⁻¹ Siemens, at least 10¹⁴ positive charges per cubic centimeter at pH 7, and a length of at least 2 namometers.

A Process for Preparing a Tubulin Isotype Database

Tubulin is a component of microtubules. At the molecular level tubulin's roles are highly complex. For example, microtubules undergo cycles of rapid growth and disassembly in a process known as “dynamic instability” that appears to be critical for microtubule function. In one embodiment, the magnetic anti-mitotic compounds of this invention are capable of disrupting and/or modifying such process of “dynamic instability,” either by interacting with one or more tubulin isotypes, and/or one or more proteins involved in the dynamics of microtubule assembly and/or disassembly, and/or the microtubules themselves.

Both the alpha and the beta forms of tubulin consist of a series of isotypes, differing in amino acid sequence, each one encoded by a different gene. See, e.g., an article by Richard F. Luduena on “The multiple forms of tublin: different gene products and covalent modifications,” Int. Rev. Cytol. 178-107-275 (1998). Reference also may be had, e.g., to U.S. Pat. No. 6,306,615 (detection method for monitoring beta-tubulin isotype specific modification); the entire disclosure of this United States patent is hereby incorporated by reference into this specification.

An interesting discussion of tubulin isotypes is also presented in published U.S. patent application 2004/0121351, the entire disclosure of which is hereby incorporated by reference into this specification. As is disclosed in this published patent application, “Microtubules are essential to the eucaryotic cell due as they are involved in many processes and functions such as, e.g., being components of the cytoskeleton, of the centrioles and ciliums and in the formation of spindle fibres during mitosis. The constituents of microtubules are heterodimers consisting of one α-tubulin molecule and one β-tubulin molecule. These two related self-associating 50 kDa proteins are encoded by a multigen family. The various members of this multigen family are dispersed all over the human genome. Both α-tubulin and β-tubulin are most likely to originate from a common ancestor as their amino acid sequence shows a homology of up to 50%. In man there are at least 15 genes or pseudogenes for β-tubulin.”

As is also disclosed in published U.S. patent application 2004/0121351, “The conservation of structure and regulatory functions among the β-tubulin genes in three vertebrate species (chicken, mouse and human) allowed the identification of and categorization into six major classes of beta-tubulin polypeptide isotypes on the basis of their variable carboxyterminal ends. The specific, highly variable 15 carboxyterminal amino acids are very conserved among the various species. Beta-tubulins of categories I, II, and IV are closely related differing only 2-4% in contrast to categories III, V and VI which differ in 8-16% of amino acid positions [Sullivan K. F., 1988, Ann. Rev. Cell Biol. 4: 687-716] . . . the expression pattern is very similar between the various species as can be taken from the following table [Sullivan K. F., 1988, Ann. Rev. Cell Biol. 4: 687-716] which comprises the respective human members of each class: 1 isotype member expression pattern class I HM 40 ubiquitous class II H β9 mostly in the brain class III H β4 exclusively in the brain class IVa H β5 exclusively in the brain class IVb H β2 ubiquitous . . . . “The C terminal end of the beta-tubulins starting from amino acid 430 is regarded as highly variable between the various classes. Additionally, the members of the same class seem to be very conserved between the various species. As tubulin molecules are involved in many processes and form part of many structures in the eucaryotic cell, they are possible targets for pharmaceutically active compounds. As tubulin is more particularly the main structural component of the microtubules it may act as point of attack for anticancer drugs such as vinblastin, colchicin, estramustin and taxol which interfere with microtubule function. The mode of action is such that cytostatic agents such as the ones mentioned above, bind to the carboxyterminal end the β-tubulin which upon such binding undergoes a conformational change. For example, Kavallaris et al. [Kavallaris et al. 1997, J. Clin. Invest. 100: 1282-1293] reported a change in the expression of of specific β-tubulin isotypes (class I, II, III, and IVa) in taxol resistant epithelial ovarian tumor. It was concluded that these tubulins are involved in the formation of the taxol resistence. Also a high expression of class III β-tubulins was found in some forms of lung cancer suggesting that this isotype may be used as a diagnostic marker.”

The function of certain tubulins in Taxol resistance was also discussed in U.S. Pat. No. 6,362,321, the entire disclosure of which is hereby incorporated by reference into this specification. As is disclosed in this patent, “Taxol is a natural product derived from the bark of Taxus brevafolio (Pacific yew). Taxol inhibits microtubule depolymerization during mitosis and results in subsequent cell death. Taxol displays a broad spectrum of tumorcidal activity including against breast, ovary and lung cancer (McGuire et al., 1996, N. Engld. J. Med. 334:1-6; and Johnson et al., 1996, J. Clin. Ocol. 14:2054-2060). While taxol is often effective in treatment of these malignancies, it is usually not curative because of eventual development of taxol resistance. Cellular resistance to taxol may include mechanisms such as enhanced expression of P-glycoprotein and alterations in tubulin structure through gene mutations in the β chain or changes in the ratio of tubulin isomers within the polymerized microtubule (Wahl et al., 1996, Nature Medicine 2:72-79; Horwitz et al., 1993, Natl. Cancer Inst. 15:55-61; Haber et al., 1995, J. Biol. Chem. 270:31269-31275; and Giannakakou et al., 1997, J. Biol. Chem. 272:17118-17125) . . . ” In one embodiment of this invention, the magnetetic anti-mitotic compound of this invention is used in conjunction with paclitaxel to provide an improved anti-cancer composition. Without wishing to be bound to any particular theory, applicants believe that their anti-mitotic compound targets a tubulin isotype that is responsible for the drug resistance to paclitaxel.

The increased presence of certain tubulin isotypes associated with certain types of cancers was noted in an article by Tien Yeh et al., “The B_{II} Isotype of Tubulin is Present in the Cell Nuclei of a Variety of Cancers,” Cell Motility and the Cytoskeleton 57:96-106 (2004). Constructs of these B_{II} isotypes and applicants' magnetic anti-mitotic compound comprise one embodiment of the present invention.

The Yeh et al. article discloses that both alpha-tubulin and beta-tubulin consist of a series of isotypes differieng in amino acid sequence, each one encoded by a different gene; and it refers to a 1998 article by Richard F. Luduena entitled “The multiple forms of tubulin: different gene products and covalent modifications,” Int. Rev. Cytol 178:207-275. The Yeh et al. article also disclosed that the B_{II} isotype of tubulin is present in the nuclei of many tumors, stating that “Three quarters (75%) of the tumors we examined contained nuclear the B_{II} (Table I).” The authors of the Yeh et al. article suggest that (at page 104) “ . . . it would be interesting to expore the possibility of using nuclear B_{II}ias a chemotherapeutic target.”

It thus appears that many isotypes of tubulin might be “chemotherapeutic targets” such as, e.g., the “nuclear B_{II}” disclosed in the Yeh et al. article, or the “ . . . specific β-tubulin isotypes (class I, II, III, and IVa) . . . ” described in the Kavallaris et al. article (Kavallaris et al. 1997, J. Clin. Invest. 100: 1282-1293) and discussed in published U.S. patent application 2004/0121351. It also appears that many isotypes of tubulin are “ . . . targets for pharmaceutically active compounds . . . .” The process of this invention may be used to identify these tubulin isotype targets, to model such targets, and to determine what therapeutic agents interact with such targets; and it may also be used to assist in the construction of anti-mitotic agents bound to such isotypes.

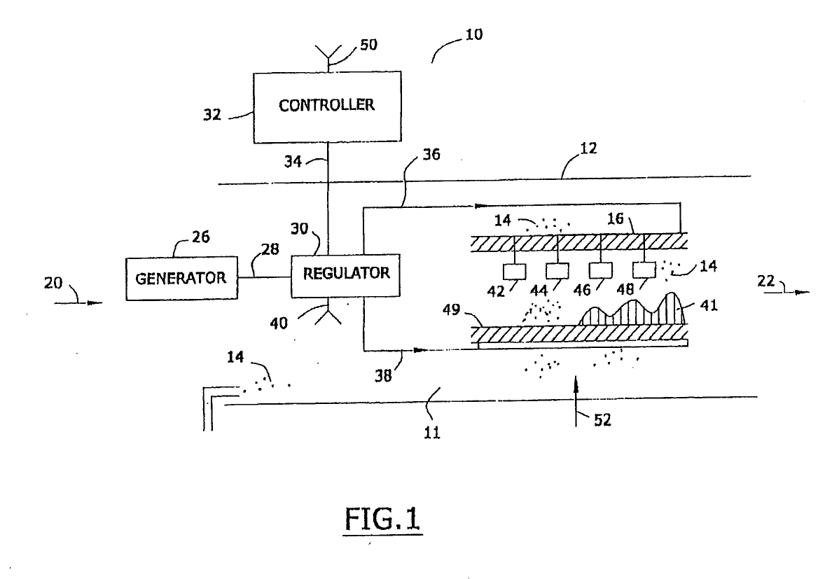

As is discussed in published United States patent application U.S. 2002/0106705 (the entire disclosure of which is hereby incorporated by reference into this specification), the therapeutic agent that interacts with the tubulin isotype target may be, e.g., a “β-tubulin modifying agent.” One such agent is described in U.S. 2002/0106705 as being “ . . . an agent that has the ability to specifically react with an amino acid residue of β-tubulin, preferably a cysteine, more preferably the cysteine residue at position 239 of a β-tubulin isotype such as β1- β2- or β4-tubulin and antigenic fragments thereof comprising the residue, preferably cysteine 239. The β-tubulin modifying agent of the invention can be, e.g., any sulfhydryl or disulfide modifying agent known to those of skill in the art that has the ability to react with the sulfur group on a cysteine residue, preferably cysteine residue 239 of a P-tubulin isotype. Preferably, the β-tubulin modifying agents are substituted benzene compounds, pentafluorobenzenesulfonamides, arylsulfonanilide phosphates, and derivatives, analogs, and substituted compounds thereof (see, e.g., U.S. Pat. No. 5,880,151; PCT 97/02926; PCT 97/12720; PCT 98/16781; PCT 99/13759; and PCT 99/16032, herein incorporated by reference; see also Pierce Catalogue, 1999/2000, and Means, Chemical Modification of Proteins). In one embodiment, the agent is 2-fluoro-1-methoxy-4-pentafluorophenylsulfonamidobenzene (compound 1; FIG. 1C). Modification of a β-tubulin isotype at an amino acid residue, e.g., cysteine 239, by an agent can be tested by treating a β-tubulin peptide, described herein, with the putative agent, followed by, e.g., elemental analysis for a halogen, e.g., fluorine, reverse phase HPLC, NMR, or sequencing and HPLC mass spectrometry. Optionally compound 1 described herein can be used as a positive control. Similarly, an α-tubulin modifying agent refers to an agent having the ability to specifically modify an amino acid residue of an α-tubulin.” In one embodiment of this invention, prior art beta-tubulin targeting agents are modified by making them water-soluble and/or magnetic in accordance with the process of this invention.

Amino acid sequencing of alpha-tubulin and beta-tubulin indicate that these tubulins are highly related. Reference may be had to, e.g., two articles by Richard F. Luduena et al. on “Isolation and partial characterization of α- and β-tublin from outer doublets of sea-urchin sperm and microtubules of chick-embryo brain” (Proc. Nat. Acad. Sci. USA 70, 3594-3598, 1973), and “α- and β-tublin: separation and partial sequence analysis” (Ann. N.Y. Acad. Sci 253, 272-283, 1975). A “Table 2.2” from the latter Luduena et al. article is presented on page 39 of Pierre Dustin's “Microtubules (Springer-Velag, New York, N.Y., 1978). At such page 39, Dustin reports that, with regard to such alpha- and beta-tubulins, “each has been highly stable in the course of evolution, as indicated by the similarities of tubulins from two widely separated species like the chick and the sea urchin: in α tubulin, no differences were found in the first 25 N-terminal amino acid . . . .”

There are, however, some distinct differences between the alpha- and beta-tubulins. As reported in the Dustin book (at pages 39-40), “It is likely that α- and β-tubulins derive from a common ancestor protein. They do differ by the location of their specific binding sites for guanine nucleotides, and the lateral and longitudinal sites necessary for their assembly into tubules. They differ also by the sites of fixation of specific poisons such as colchicines and VLB . . . .”

It has also been reported that most, but not necessarily all, microtubules are comprised of identical alpha/beta dimmers. As is disclosed on page 40 of the Dustin book, “Electrophoretic data indicate that the two tubulins are present in equal quantities in most MT studied. The ultrastructural data suggest that MT are assembled from identical, αβ dimmers . . . . If solubulized tubulin is treated with a cross-linking agent . . . and studied on an acrylamide gel system capable of discriminating between αα, αβ, and ββ dimmers, it is found that most tubulin is of the αβ type.”

There is a difference in charge between alpha and beta tubulins which allows for their separation. As is also disclosed on page 39 of the Dustin text, “The dimeric structure of tubulin was early recognized . . . and amply confirmed by research on the fixation of colchicines and VLB, and the location of the guanine nucleotides. The polyacrylamide gel electrophoresis of tubulin preparations shows two closelhy located bands, namely α- and β-tubulins, the β subunit having the greater electrophorectic mobility . . . . This separation results from a difference of charge.”

Identification of the Tubulin Isotype Targets

The tubulin isotypes that are potential chemotherapeutic targets are preferably those isotypes that are present in a higher concentration in diseased biological organisms than in normal biological organisms. They may be identified by, e.g., standard analytical techniques.

By way of illustration, and not limitation, an analysis may be done regarding the extent to which, if any, a beta-tubulin isotype, e.g., is present in tumors. As is described in the Yeh et al. paper cited elsewhere in this specification, one may study a variety of tumors by “standard immunohistochemical techniques” to determine the extent to which one or more tubulin isotypes if present in the tumors. Yeh et al. state that: “Tumors were randomly selected from the San Antonio Cancer Institute Tumor Bank to represent a variety of tumor types, grades, and stages. Benign tissues adjacent to the tumor were examined when possible. In addition to malignant tumors, selected benign lesions, such as meningiomas, and tumors of low malignant potential, such as giant cell tumors of bone, were also examined. All tissues were formalin-fixed and paraffin-embedded . . . Standard immunohistochemical techniques were utilized [Hsu et al., 1981]. The monoclonal antibody to the (BII isotype of tubulin (JDR.3B8) was at an initial concentration of 2 mg/mL and diluted 1:2,000, for a final concentration of 1 μg/mL. No antigen retrieval step was used because the antigen was easily accessible for immunohistochemical staining. Slides were incubated at room temperature with the primary antibody for 1 h. The sections were then exposed to a secondary biotinylated rabbit anti-mouse antibody (DAKO, cat no. E354, 1:100), then Streptavidin horseradish peroxidase was applied, followed by diaminobenzidine and OsO4. Slides were counter-stained with methyl green. A positive skin control and negative controls (minus antibody) were run with each batch of tumors . . . . Slides were visualized using an Olympus BX-40 microscope, equipped with PlanFluorite objectives. The pattern and location of cells staining with the antibody to B11-tubulin were recorded. Intensity and proportion of cells stained were recorded in a semi-quantitative manner, as previously described [Allred et al., 1998] . . . .”

Preparation of a Database of Tubulin Isotypes

In one embodiment of the process of this invention, a database of tubulin isotypes is prepared. In this section of the specification, excerpts from a paper that was prepared by one of the applicants is presented. The paper in question is entitled “Homology Modeling of Tubulin Isotypes and its Consequences for the Biophysical Properties of Tubulin and Microtubules.” One of the authors of this paper is applicant Jack .A. Tuszynski; and such paper will hereinafter be referred to as the “Tuszynski paper.”.

As is disclosed in the introductiory portion of the Tuszynski et al. paper, “Microtubules, cylindrical organelles found in all eukaryotes, are critically involved in a variety of cellular processes including motility, transport and mitosis.” As authority for this proposition, the paper cites a text by J. S. Hymans et al. entitled “Microtubules” (Wiley-Liss, New York, N.Y., 1994).

The Tuszynski paper also discloses that: “ ” Their component protein, tubulin, is composed of two polypeptides of related sequence, designated α and β. In addition to α- and β-tubulin, many microtubules in cells require the related γ-tubulin for nucleation.” As authority for this proposition, there are cited articles by H. P. Erickson (“γ-tubulin nucleation, template or protofilament?,” Nature Cell Biology 2:E93-E96, 200) and by R. F. Luduena (“The multiple forms of tubulin: different gene products and covalent modifications,” Int. Rev. Cytol. 178:207-275; 1998).

The Tuszynski paper also discloses that: “Two other tubulins, designated δ and ε, are widespread, . . . although their roles are still uncertain . . . models utilizing them have been proposed.” As authority for this statement, the paper cites works by S. T. Vaughan et al. (“New tubulins in protozoal parasites,” Curr. Biol. 10:R258-R259, 2000) and Y. F. Inclan et al. (“Structural models for the self-assembly and microtubule interactions of . . . tubulin,” Journal of Cell Science 114:413-422, 2001).

The Tuszynski paper also discloses that: “At least three of these tubulins, namely, α, β, and γ, exist in many organisms as families of closely related isotypes. An enigmatic feature of tubulin is its heterogeneity. Not only can α- and β-tubulin exist as multiple isotypes in many organisms, but the protein can also undergo various post-translational modifications, such as phosphorylation, acetylation, detyrosination, and polyglutamylation.” As authority for this statement, the paper cites a work by A. Banergee, “Coordination of posttranslational modificatioins of bovine brain. α-tubulin, polyglycylation of delta2 tubulin,” Journal of Biological Chemistry 277:46140-46144, 2002).

The Tuszynski paper also discloses that “At the molecular level tubulin's roles are highly complex and are related to the structural variations observed.” As authority for this proposition, the article cites a work by K. L. Richards et al., “Structure-function relationships in yeast tubulins,” Molecular Biology of the Cell 11:1887-1903, 2000.

The Tuszynski paper also states that “ . . . microtubules undergo cycles of rapid growth and disassembly in a process known as dynamic instability that appears to be critical for microtubule function, especially in mitosis. A guanosine triphosphate (GTP) tubulin hydrolyzes bound GTP to GDP; the kinetics of this process in beta-tubulin is critical in regulating dynamic instability by affecting the loss of a so-called ‘cap’ that stabilizes the microtubule structure.” As authority for this statement, the article cites a work by T. J. Mitchison et al., “Dynamic instability of microtubule growth,” Nature 312:237-242, 1984.

The Tuszynski paper also discloses that “In addition to forming microtubules, tubulin interacts with a large number of associated proteins. Some of these, such as tektin, may play structural roles; others, the so-called microtubule-associated proteins (MAPs) such as tau or MAP2, may stabilize the microtubules, stimulate microtubule assembly and mediate interactions with other proteins. Still others, such as kinesin and dynein, are motor proteins that move cargoes, e.g., vesicles, along microtubules.” As authority for these statements, the article refers to works by M. Kikkawa et al. (“Switch-based mechanisms of kinesin motors,” Nature 411:439-445, 2001) and Z. Wang et al. (“The C-terminus of tubulin increases cytoplasmic dynein and kinesin processity,” Biophysical Journal 78:1955-1964, 2000).

As is also disclosed in the Tuszynski et al. paper, “The precise molecular basis of the properties of tubulin is still not well understood, in part because tubulin's highly flexible conformation . . . makes it difficult to crystallize this region.” As authority for this statement, the article cites a work by O. Keskin et al., “Relating molecular flexibility to function: a case study of tubulin,” Biphysical Journal 83:663-680, 2002.

The Tuszynski paper also discloses that: “In a major advance in the field, the three-dimensional structure of bovine brain tubulin has been determined by electron crystallography resulting in atomic structures available in the The Protein Data Bank (Berman et al. [2000] as entries 1TUB Nogales et al. (1998) and 1JFF Lowe et al. (2000).” The Berman et al. reference is to an article by H. M. Berman et al. on “The protein data bank,” Nucleic Acids Research 28:235-242, 2000. The Nogales et al. reference was to an article by E. Nogales et al. on the “Structure of the alpha/beta tubulin dimer by electron crystallography,” Nature 393: 199-203, 1998. The Lowe et al. reference is to an article by J. Lowe et al. on the “Refined structure of alpha/beta-tubulin at 3.5 angstrom resolution,” Journal of Molecular Biology 313:1045-1057 (2001).

The Tuszynski paper also discloses that “Once the three dimensional structure of a protein is known it is possible to use homology modeling to predict the structures of related forms of the protein with some degree of accuracy. We have applied these techniques to a series of 300 different tubulins, representing α- and β-tubulins from animals, plants, fungi and protists, as well as several γ-, δ- and ε-tubulins.” It should be noted that such “homology modeling” is frequently referred to in the patent literature. Reference may be had, e.g., to U.S. Pat. Nos. 5,316,935; 5,486,802; 5,686,255; 5,738,998; 6,027,720; 6,080,549; 6,197,589; 6,356,845; 6,433,158; 6,451,986; 6,468,770; 6,548,477; 6,654,644; 6,654,667; 6.627,746; and the like. The entire disclosure of each of these United States patents is hereby incorporated by reference into this specification.

The Tuszynski paper also discloses that: “For all of the resulting tubulin structures, we have been able to estimate the magnitudes and orientations of their dipole moments, charge distributions and surface to volume ratios. The magnitudes and orientations of the tubulin dimers' dipose moments appear to play significant roles in microtubule assembly and stability.”

The Tuszynski paper also discloses that “In addition, we have been able to generate plausible conformations for the C-terminal regions. Notably, the C-termini of alpha- and beta-tubulin were not resolved in the original crystallographic structures of tubulin due to their flexibility and possibly sample inhomgeneity.” As support for this statement, the article cited a work by E. Nogales et al., “Structure of the alpha/beta tubulin dimmer by electron crystallography,” Nature 393:199-203, 1998.

The Tuszynski paper also discloses that “The importance of these regions is highlighted by the fact that they are the site of most of tubulin's post-translational modifications, that they bind to MAPs and that differences among tubulin isotypes cluster here.”

The Tuszynski paper discusses the materials and methods used to construct the tublin isotype database. In one embodiment of the process used in the Tuszynksi paper, the “ . . . abundance of various homologous isotypes of tubulin, called alpha and beta (with additional indices labeling the isotypes) is correlated with the specific locations of the cells in which they are found. We have used the known amino-acid sequences in which the isotypes differ, in connection with the data of the Downing group for the known three-dimensional structure obtained by electron crystallography of bovine brain tubulin by Nogales et al., and applied these in molecular dynamics simulations in order to study the resulting differences in the biophysical and biochemical properties such as: volume, surface are, electric field distributions, binding sites, conformational changes, etc. Our structural experiments on purified abII, abIII and abIV tubulin dimers have produced strong evidence that their conformations differ. Using the Molecular Simulation International (MSI) Homology Software Module, we have constructed three-dimensional models of the abI, abII, abIII, abIV, abV, abVI and abVII dimers. This Downing structure was fitted to the amino acid sequences for porcine brain a- and b-tubulin, which, for the beta subunit, is largely bII. To generate models of the various dimers, the Homology software module is used to align the sequences of the various isotypes to the sequence of the Nogales et al structure, and the coordinates of the Nogales structure are mapped to the aligned beta isotype. Then energy minimization and molecular dynamic simulation is being used on the approximate result to refine a structural model of each of these dimers. Similar homology modeling approaches have been used to predict the structure of one protein from that of a closely related protein; such models have also been extensively used to design useful drugs. In constructing computational 3D models from all of the available sequences of tubulin isotypes we have exploited the high degree of sequence and structure conservation that is observed within tubulin isotypes and between the alpha and beta subunits by using software such as the experimental Modeller and tubulin crystallographic data as structural templates to produce 3D models containing chosen amino acid sequences.”

In one embodiment of the Tuszynski process, the “Swiss-Prot database” was referred to. As is also disclosed in the Tuszynski paper, “As an initial step the Swiss-Prot database Release 40.2 of 08 Nov. 2002 . . . (available at http://www.expasy.org/sprot/]) was searched for tubulin amino acid sequences.” The article referred to a work by B. Boekmann et al. (“The SWISS—PROT protein knowledgebase and its supplement TrEMBL,” Nucl. Acids. Res. 31:365-370, 2003) for a reference relating to such “Swiss-Prot database.” It should be noted that many United States patents refer to such Swiss-Prot database. Reference may be had, e.g., to U.S. Pat. Nos. 6,183,968; 6,207,397; 6,303,319; 6,372,897; 6,373,971 (method and apparatus for pattern discovery in protein sequences); U.S. Pat. Nos. 6,387,641; 6,631,322 (methods for using functional site descriptors and predicting protein function), U.S. Pat. No. 6,466,874 (Rosetta stone method for detecting protein function and protein-protein interactions from genome sequences), U.S. Pat. No. 6,470,277 (techniques for facilitating identification of candidate genes), U.S. Pat. No. 6,564,151 (assigning protein functions by comparative genome analysis protein phylogenetic profiles), and the like. The entire disclosure of each of these United States patents is hereby incorporated by reference into this specification.

Referring again to the Tuszynksi paper, it is disclosed that: “A search using the keyword ‘tubulin’ was manually filtered to separate actual tubulin sequences from those of other tubulin related proteins. This provided some 290 sequences, representing a wide range of species. Of these 27 are annotated as being fragmentary, leaving 263 complete tubulin monomer sequences. Of particular interest were the 15 human sequences obtained . . . .”

Referring again to the Tuszynksi paper, it is disclosed that: “Table 1 summarizes all of the tubulin sequences used in this study for quick reference and convenience. The table names the source organism, and for each . . . gives the name used in the databank. It is important to relate the biochemical data encapsulated by the amino acid sequence to the biologically relevant information presented in Table 1 in the form of the organism from which a given tubulin is derived.”

In referring to such “Table 1,” the Tuszynski paper states that: “Table 1. Tubulin sequences used in this study. The table names the source organism, and for each . . . gives the name used in the databank.”

For “Animals,” such Table 1 listed the following source organisms: Haemonchus contortus a: TBA_HAECO; Caenorhabditis briggsae b: TBB7_CAEBR; Caenorhabditis elegans a: TBA2_CAEEL, TBA8_CAEEL; b: TBB2_CAEEL, TBB4_CAEEL, TBB7_CAEEL; g: TBG_CAEEL; Brugia pahangi b: TBB1_BRUPA; Onchocerca gibsoni b: TBB_ONCGI; Homarus americanus (American lobster) a: TBA1_HOMAM, TBA2_HOMAM, TBA3_HOMAM; b: TBB1_HOMAM, TBB2_HOMAM; Bombyx mori (Domestic silkworm) a: TBA_BOMMO, b: TBB_BOMMO; Manduca sexta (Tobacco hawkmoth) b: TBB1_MANSE; Drosophila erecta (fruit fly) b: TBB2_DROER; Drosophila melanogaster (fruit fly) a: TBA1_DROME; TBA2_DROME, BA3_DROME, TBA4_DROME; b: TBB2_DROME, TBB3_DROME; g: TBG2_DROME; Patella vulgata (common limpet) a: TBA2_PATVU; Haliotis discus (Pacific black abalone) b: TBB_HALDI; Octopus dofleini (giant Pacific octopus) a: TBA_OCTDO; b: BB_OCTDO; Lymnae stagnalis (giant pond snail) b: TBB_LYMST; Octopus vulgaris (common octopus) a: TBA_OCTVU; Lytechinus pictus (painted urchin) a: TBA_LYTPI, b: TBB_LYTPI; Paracentrotus lividus (common sea urchin) a: TBA1_PARLI; b: BB_PARLI; Strongylocentrotus purpuratus (purple sea urchin) b: TBB_STRPU; Onchorhynchus keta (chum salmon) a: TBAT_ONCKE; Onchorhynchus mykiss (rainbow trout) a: TBAT_ONCMY; Gadus morhua (Atlantic cod) b: TBB1_GADMO; Notothenia coriiceps b: TBB1_NOTCO; Pseudopleuronectes americanus (winter flounder) b: TBB_PSEAM; Torpedo marmorata (electric eel) a: TBA_TORMA; Notophthalmus viridiscens (Eastern newt) a: TBA_PATVI; Xenopus laevis (African clawed frog) a: TBA_XENLA; b: TBB2_XENLA, TBB4_XENLA; g: TBG_XENLA; Gallus gallus (chicken) a: TBA1_CHICK, TBA2_CHICK, TBA3_CHICK, TBA4_CHICK, TBA5_CHICK, TBA8_CHICK; b: TBB1_CHICK, TBB2_CHICK, TBB3_CHICK, TBB4_CHICK, TBB5_CHICK, TBB6_CHICK, TBB7_CHICK; Mus musculus (house mouse) a: TBA1_MOUSE, TBA2_MOUSE, TBA3_MOUSE, BA6_MOUSE, TBA8_MOUSE; g: TBG1_MOUSE, TBG2_MOUSE; Rattus norvegicus (Norway rat) b: TBB1_RAT; Sus scrofa (pig) a: TBA_PIG; b: TBB_PIG; Homo sapiens (human) a: TBA1_HUMAN, TBA2_HUMAN, TBA4_HUMAN, TBA6_HUMAN, TBA8_HUMAN; b: TBB1_HUMAN, TBB2_HUMAN, TBB4_HUMAN, BB5_HUMAN, TBBQ_HUMAN, TBBX_HUMAN; g: TBG1_HUMAN, TBG2_HUMAN; d: TBD_HUMAN; e: TBE_HUMAN.”

Referring again to Table 1 of the Tuszynski paper, the following source organisms were listed for “Plants:” Cyanaphora paradoxa b: TBBA_CYAPA; Physcomitrella patens ( )g: TBG_PHYPA; Anemia phyllitidis (flowering fern) a: TBA1_ANEPH, TBA2_ANEPH; b: TBB1_ANEPH, TBB2_ANEPH, TBB3_ANEPH; g: TBG_ANEPH; Picia abies (Norway spruce) a: TBA_PICAB; Zea mays (maize) a: TBA1_MAIZE, TBA2_MAIZE, TBA3_MAIZE, TBA4_MAIZE, TBA5_MAIZE, TBA6_MAIZE; b: TBB1_MAIZE, TBB2_MAIZE, TBB3_MAIZE, TBB4_MAIZE, TBB5_MAIZE, TBB6_MAIZE, TBB7_MAIZE, TBB8_MAIZE; g: TBG1_MAIZE, TBG2_MAIZE, TBG3_MAIZE; Eleusine indica (goosegrass) a: TBA1-ELEIN, TBA2-ELEIN, TBA3-ELEIN; b: TBB1-ELEIN, TBB2-ELEIN, TBB3-ELEIN, TBB4-ELEIN; Hordeum vulgare (barley) a: TBA1_HORVU, TBA2_HORVU, TBA3_HORVU; b: TBB_HORVU; Triticum aestivum (bread wheat) a: TBA_WHEAT; b: TBB1_WHEAT, TBB2_WHEAT, TBB3_WHEAT, TBB4_WHEAT, TBB5_WHEAT; Pisum sativus (pea) a: TBA1_PEA; b: TBB1_PEA, TBB2_PEA, TBB3_PEA; Prunus dulcis (almond) a: TBA_PRUDU; Arabidopsis thaliana (thale cress) a: TBA1_ARATH, TBA2_ARATH, TBA3_ARATH, TBA6_ARATH; b: TBB1_ARATH, TBB2_ARATH, TBB4_ARATH, TBB5_ARATH, TBB6_ARATH, TBB7_ARATH, TBB8_ARATH, TBB9_ARATH; g: TBG2_ARATH; Avena sativa (oat) a: TBA_AVESA; b: TBB1_AVESA; Oryza sativa (rice) a: TBA1_ORYSA; b: TBB1_ORYSA, TBB2_ORYSA, TBB3_ORYSA; g: TBG2_ORYSA; Daucus carota (carrot) b: TBB1_DAUCA, TBB2_DAUCA; Glycine max (soybean) b: TBB1_SOYBN, TBB2_SOYBN, TBB3_SOYBN; Solanum tuberosum (potato) b: TBB1_SOLTU, TBB2_SOLTU; Cicer arietinum (chickpea) b: TBB_CICAR; Lupinus albus b: TBB1_LUPAL, TBB2_LUPAL.”

Referring again to the Tuszynski paper, the following “source organisms” were listed for “Fungi” and “Yeast:” “Emericella nidulans a: TBA1_EMENI, TBA2_EMENI; b: TBB1_EMENI, TBB2_EMENI; g: TBG_EMENI; Mycosphaerella graminicola a: TBA_MYCGR; eurospora crassa a: TBA1_NEUCR, TBA2_NEUCR; b: TBB_NEUCR; g: TBG_NEUCR; Glomerella cingulata b: TBB1_COLGL, TBB2_COLGL; Glomerella graminicola b: TBB1_COLGR, TBB2_COLGR; Sordaria macrospora a: TBA_SORMA; Ajellomyces capsulatum a: TBA_AJECA, b: TBB_AJECA; Pneumocystis carinii a: TBA1_PNECA, TBAA_PNECA; b: TBB_PNECA; Aspergillus flavus b: TBB_ASPFL; Aspergillus parasiticus b: TBB_ASPPA; Erysiphe pisi b: TBB2_ERYPI; Botryotinia fuckeliana b: TBB_BOTCI; Blumeria graminis b: TBB_ERYGR; Mycosphaerella pini b: TBB_MYCPJ; Venturia inaequalis b: TBB_VENIN; Phaeosphaeria nodorum b: TBB_PHANO; Rhynchosporium secalis b: TBB_RHYSE; Penicillium digitatum b: TBB_PENDI; Pestalotiopsis microspora b: TBB_PESMI; Neotyphodium coenophialum b: TBB_ACRCO; Epichloe typhina b: TBB_EPITY; Gibberella fujikuroi b: TBB_GIBFU; Acremonium chrysogenum b: TBB_CEPAC; Trichoderma viride b: TBB1_TRIVI, TBB2_TRIVI; Cochlioboius heterostrophus g: TBG_COCHE; Candida albicans a: TBA_CANAL; b: TBB_CANAL; g: TBG_CANAL; Saccharomyces cerevisiae a: TBA1_YEAST, TBA3_YEAST; b: TBB_YEAST; g: TBG_YEAST; Schizosaccharomyces pombe a: TBA1_SCHPO, TBA2_SCHPO; b: TBB_SCHPO; g: TBG_SCHPO; Schizosaccharomyces japonicus g: TBG_SCHJP; Galactomyces geotrichum b: TBB1_GEOCN, TBB2_GEOCN; Schizophyllum commune a: TBAA_SCHCO, TBAB_SCHCO; b: TBB_SCHCO; Pleurotus sajor-caju b: TBB_PLESA; Microbotryum violaceum g: TBG_USTVI.”

Referring again to the Tuszynski paper, the following “source organisms” were listed in Table 1 for “Protists:” “Chlamydomonas reinhardtii a: TBA1_CHLRE, TBA2_CHLRE; b: TBB_CHLRE; g: TBG_CHLRE; Chlamydomonas incerta reinhardtii b: TBB_CHLIN; Volvox carteri a: TBA1_VOLCA; b: TBB1_VOLCA; Chlorella vulgaris a: TBA_CHLVU; Polytomella agilis b: TBB_POLAG; Stylonichia lemnae a: TBA1_STYLE, TBA2_STYLE; b: TBB_STYLE; Oxytricha granulifera a: TBA_OXYGR; Tetrahymena pyriformis a: TBA_TETPY; b: TBB_TETPY; Tetrahymena thermophila a: TBA_TETTH; b: TBB_TETTH; Paramecium tetraurelia b: TBB1_PARTE; Euplotes aediculatus g: TBG_EUPAE; Euplotes focardii b: TBB_EUPFO; Euplotes octocarinatus a: TBA_EUPOC; b: TBB_EUPOC; g: TBG2_EUPOC; g: TBG2_EUPOC; Euplotes vannus a: TBA_EUPVA; Monoeuplotes crassus a: TBB_EUPCR; g: TBG2_EUPCR; Blepharisma japonicus a: TBA_BLEJA; Plasmodium falciparum a: TBA_PLAFK; b: TBB_PLAFK, TBB_PLAFA; g: TBG_PLAFO; Plasmodium berghei yoelii a: TBA_PLAYO; Toxoplasma gondii a: TBA_TOXGO; b: TBB_TOXGO; Babesia bovis b: TBB_BABBO; Eimeria tenella b: TBB_EIMTE; Naegleria gruberi a: TBA_NAEGR; b: TBB_NAEGR; Trypanosoma brucei a: TBA_TRYBR; b: TBB_TRYBR; Trypanosoma cruzi a: TBA_TRYCR; b: TBB_TRYCR; Leishmania mexicana b: TBB_LEIME; Leptomonas seymouri a: TBA_LEPSE; Euglena gracilis a: TBA_EUGGR; b: TBB_EUGGR; Physarum polycephalum a: TBAD_PHYPO, TBAE_PHYPO, TBAN_PHYPO; b: TBB1_PHYPO; TBB2_PHYPO; Pelvetia fastigiata a: TBA1_PELFA, TBA2_PELFA; Entamoeba histolytica a: TBA1_ENTHI; g: TBG_ENTHI; Dictyostelium discoideum a: TBA_DICDI; b: TBB_DICDI; Giardia intestinalis b: TBB_GIALA; Reticulomyxa filosa g: TBG_RETFI; Porphyra purpura b: TBB1_PORPU, TBB2_PORPU, TBB3_PORPU, TBB4_PORPU; Ectocarpus variabilis b: TBB5_ECTVR, TBB6_ECTVR; Achlya klebsiana b: TBB_ACHKL; Phytophthora cinnamomi b: TBB_PHYCI; Thalassiosira weisflogii b: TBB_THAWE; Chondrus crispus b: TBB1_CHOCR.”

Referring again to the Tuszynksi paper, and in referring to “Model Construction,” the paper disclosed that: “The structures of alpha and beta tubulins are known to be quite similar, being nearly indistinguishable at 6 Angstroms . . . dispite only a 40% amino acid homology.”.” As support for this statement, reference is made to an article by H. Li et al., “Microtubule structure at 8 angstrom resolution,” Structure 10:1317-1328, 2002.”

Referring again to the Tuszynksi paper, it is disclosed that: “ . . . Since the sequences within an alpha or beta tubulin family are more similar to each other than to those sequences belonging to the other families of tubuins, it is reasonable to believe that any given sequence should produce a structure very similar to another member of a given family. Further support for this comes from the published structures of Nogales et al. (1998) and Lowe et al. (2001) which are of a porcine sequence, but which were fit to data from an inhomogeneous bovine sample.” The Nogales et al. reference is to an article by E. Nogales et al., “Structure of the alpha/beta tubulin dimmer by electron crystallogaraphy,” Nature 393: 199-303. The Lowe et al. reference was to an article by J. Lowe et al., “Refined structure of alpha/beta1 tubulin at 3.5 angstrom resolution,” Journal of Molecular Biology 313:1045-1057 (2001).

Referring again to the Tuszynksi paper, it is disclosed that: “Accordingly, by substituting appropriate amino acid side chains and properly adjusting other residues to accommodate insertions and deletions and in the sequence, crystallographic structures can be used as a framework to produce model structures with different sequences with a high degree of confidence.”

As is also disclosed in the Tuszynski et al. paper, “To build such 3D structures of the many isotypes Modeller (version 6.2) was used [Marti-Renom 2000].” The Marti-Renom reference is an article by M. A. Marti-Renom et al., “Comparative protein structure modeling of genes and genomes,” Annu. Rev. Biophys. Biomol. Struct. 29:291-325, 2000.

In the Marti-Renom paper, it is stated that the MODELLER database is disclosed at “guitar.Rockefeller.edu/modeler.html” and is discussed in an article by A. Sali et al., “Comparative protein modeling by satisfaction of spatial restraints,” J. Mol. Biol. 234:799-915, 1993.

The Modeller database is also referred to in the patent literature. Reference may be had, e.g., to U.S. Pat. Nos. 5,859,972; 5,968,782; 5,985,643; 6,225,446; 6,251,620 (three dimensional structure of a ZAP tyrosine protein kinase fragement and modeling methods), U.S. Pat. Nos. 6,391,614; 6,417,324; 6,459,996; 6,468,772; 6,495,354; 6,495,674; 6,532,437; 6,559,297; 6,605,449; 6,642,041; 6,607,902; 6,645,762; 6,569,656; 6,677,377 (structure based discovery of inhibitors of matriptase for the treatement of cancer and other conditions), U.S. Pat. No. 6,680,176; and the like. The entire disclosure of each of these United States patents is hereby incorporated by reference into this specification.

The Modeller database may be used for the “comparative protein structure modeling” that is discussed in, e.g., the Marti-Renom paper (and also in the Tuszynski paper). Such “comparative protein structure modeling” is also referred to in the patent literature. Reference may be had, e.g., to U.S. Pat. Nos. 6,462,189; 6,703,199; and 6,703,901; reference may also be had to published United States patent applications 2002/0045578 and 2004/0014944 (method and system useful for structural classification of unknown polypeptides); and reference also may be had to international patent publications WO0135255 (large scale comparative protein structure modeling); WO0234877; WO03019183(process for the informative and iterative design of a gene-family screening library), and WO03029404. The entire disclosure of each of these United States patents, of each of these published United States patent applications, and of each of these international patent applications, is hereby incorporated in its entirety into this specification.

Referring again to the Tuszynksi paper, and to the Modeller program used therein, it is disclosed that: “To build the library of 3D tubulin structures, Modeller (version 6v2) was used. This program uses alignment of the sequences with known related structures, used as templates, to obtain spatial constraints that the output structure must satisfy. Additional restraints derived from statistical studies of representative protein and chemical structures are also used to ensure a physically probable result. Missing loop regions are predicuted by simulated annealing optimization of a molecular mechanics model.”

As is known to those skilled in the art, a system as large as tubulin may have many local energy minima; thus, an energy minimization program may not be sufficient to find the lowest global minimum. To seek the difference in conformation between GTP (guanosine triphosphate) and GDP (guanosine diphosphate) tubulin, applicants preferably use an annealing procedure in which the molecule is heated up well beyond physiological temperatures to induce a difference in conformation and is then slowly cooled down below physiological temperatures. The cooling process is maintained at a low enough rate so that the molecule can move between minima and find a lower energy final conformation. For a similar process that is applied by kinesin, reference may be had, e.g., to an article by W. Wriggers et al. on “Nucleotide-dependent movements of the kinesis motor domain predicted by simulated annealing,” Biophys. J., 75:646-661, August, 1998.

In one embodiment of the process of this invention, the TINKER molecular simulation software is used. This software package is described, e.g., in an article by M. J. Dudek et al. on the “Accurate modeling of the intramolecular electrostatic energy of proteins,” J. Comput. Chem, 16:791-816, 1995. This TINKER software is also described in, e.g., U.S. Pat. Nos. 5,049,390; 6,180,612; 6,531,306; 6,537,791; and 6,573,060. The entire disclosure of each of these United States patents is hereby incorporated by reference into this specification.

In one embodiment, the TINKER anneal program is preferably used to heat up the proteins from 1 degree Kelvin to 400 degrees Kelvin and then cool them very slowly to 200 degrees Kelvin.

In one embodiment, the anneal program is used to heat up the proteins from a temperature of from about 1 to about 299 degrees Kelvin to to a temperature within the range of from about 300 to about 500 degrees Kelvin linearly over a period of from about 100 to about 100,000 picoseconds, preferably, overa period of at least about 200 picoseconds.

Referring again to the Tuszynksi paper, it is disclosed therein that “Since the 3D structures of tubulin lack the extreme C-termini of the proteins, we used this capability to create structure files that include the C-terminal amino acids by including those portions of the sequence in the Modeller input.” In the process of this invention, the tubulin with its C-terminii, “tubulin-C,” may be generated by adding the missing residues onto the alpha band beta-tubulin. Thus, e.g., one may use the “MOLMOL” software to add the “missing residues.” See, e.g., an article by R. Koradi, “MOLMOL: a program for display and analysis of macromolecular structures,” J. Mol. Graphics, 14:51-55, 1996. Reference also may be had, e.g., to U.S. Pat. No. 6,077,682 (method of identifying inhibitors of sensor histidine kinases through rational drug design); U.S. Pat. Nos. 6,162,627; 6,171,804 (method of determining interdomain orientation and changes of interdomain orientation on ligaton), U.S. Pat. No. 6,723,697; and the like. The entire disclosure of each of these United States patents is hereby incorporated by reference into this specification.

In the process described in the Tuszynski paper, the missing residues were added by the Modeller software, and the “tubulin-C model” was then subjected to an energy minimization program. As is known to those skilled in the art, in an energy minimization program, one searches for the minimum energy configuration of a molecule by moving down a gradient through configuration space (see W. F. van Gusteren et al., “Computer simulation of molecular dynamics: Methodoly, applications and perspectives in chemistry,” Angew. Chem. Int. Ed. Engl., 29-992-1023, 1990. Reference also may be had, e.g., to U.S. Pat. No. 5,453,937 (method and system for protein modeling); U.S. Pat. No. 5,5576,535 (method and system for protein modeling); U.S. Pat. No. 5,884,230 (method and system for protein modeling); U.S. Pat. No. 6,188,965 (apparatus and method for automated protein design); U.S. Pat. No. 6,269,312 (apparatus ad method for automated protein design); U.S. Pat. Nos. 6,376,504; 6,380,190; 6,403,312 (protein design authoamtic for protein libraries); U.S. Pat. Nos. 6,514,729; 6,545,152; 6,682,923; 6,689,793; 6,708,120 (apparatus and method for automated protein design); U.S. Pat. Nos. 6,746,853; 6,750,325; and the like. The entire disclosure of each of these United States patents is hereby incorporated by reference into this specification.

Referring again to the Tuszynski paper, it is disclosed that: “For our work we used five structures from the tubulin family as templates. One of these from PDB file 1FSZ (Lowe and Amos, 1998) is the crystal structure of FtsZ, a putative prokaryontic homolog of tublin Erickson (1997).” The Lowe and Amos reference is an article by J. Lowe et al., “Crystal Structure of the bacterial cell-division protein FtsZ,” Nature, 393:203-206, 1998. The Erickson reference is an article by H. P. Erickson, “FtsZ, a tubulin homologue, in prokaryote cell division,” Trends Cell Biol., 7:362-367, 1997. Reference also may be had, e.g., to U.S. Pat. No. 6,350,866, the entire disclosure of which is hereby incorporated by reference in to this specification.

Another two of the tubulin templates described in the Tuszynski paper were described as being “Two more structures (and alpha- and a beta-monomer) came from 1TUB (Nogales et al., 1998), the original tubulin crystal . . . ” The Nogales et al. reference is E. S. Nogales et al., “Structure of the alpha/beta tubulion dimmer by electron crystallography,” Nature 393:199-203, 1998.

Yet another two of the tubulin templates described in the Tuszynski paper were “ . . . two more from 1JFF (Lowe et al. 2001), a more refined version of the same structure.” The Lowe et al. reference is an article by J. H. Lowe et al. on “Refined structure of alpha/beta tubulin at 3.5 angstrom resolution,” Journal of Molecular Biology, 313:1045-1057, 2001.

As is also disclosed in the Tuszynski et al. paper, “With the resulting library of structural tubulin models, various computational estimates of physical properties of the different tubulins may be made. These include the volume, surface area, net charge, and dipole moments. We performed these calculations on the model structures, typically using analysis tools within the Gromacs (Lindahl et al., 2001) molecular dynamics package (version 3.1..4) . . . .” The Lindahl et al. reference was an article by E. B. Lindahl et al. entitled “GROMACS 3.0: A package for molecular simulation and trajectory analysis,” J. Mol. Mod., 7:306-317, 2001. Reference also may be had, e.g., to published United States patent applications 20030082521, 20030108957, 20030187626 (method for providing thermal excitation to molecular dynamics models), and 20030229456 (methods for pedicting properties of molecules). The entire disclosure of each of these published patent applications is hereby incorporated by reference into this specification.

As is also disclosed in the Tuszynski article, “We also analyzed the properties of the C-terminal projection. We first needed to define this region. We used Clustal W (version 1.82) (Thompson et al., 1994) in order to obtain a multiple sequence alignment amongst the peptides. The multiple alignment then allows rapid identification of corresponding residues in all of the sequences.” The Thompson et al. reference is an article by J. D. Thompson et al. on “CLUSTAL W: Improving the sensitivity of progressive multiple sequence alignment through sequence weighting, positions-specific gap penalties and weight matrix choice,” Nucleic Acids Research, 22:4673-4680, 1994. Reference also may be had, e.g., to U.S. Pat. Nos. 6,403,558; 6,451,548; 6,465,431; 6,489,537; 6,559,294; 6,582,950; 6,632,621; 6,653,283; 6,586,401; 6,589,936; 6,734,283; and the like. The entire disclosure of each of these United States patents is hereby incorporated by reference into this specification.

As is also disclosed in the Tuszynski paper, “Other interesting properties of tubulin are inherent to dimers. In order to create a set of dimers for study we fit an alpha-monomer and a beta-monomer to their corresponding monomers in the 1JFF structure. This was done by rotation and translation of the Modeller structures in order to minimize the RMSD between a set of alpha-carbons from residues present in all the sequences. This procedure does notprevent steric conflicts between the two monomers and can create dimers with overlaps. However, for some types of calculations such as estimates of multipiole components, this will not prevent reasonable results. A set of over 200 dimers was obtained in this way by constructing all the alpha-beta pairs that share a common species identifier in the Swiss-Prot name. This restricts the number of dimers to a manageable set and voids hybrids such as a carrot/chicken crossing that would not occur naturally.”

As is also disclosed in the Tuszynski paper, “The library of tubulin structures . . . were analyzed by molecular mechanics to determine their net charges, dipole moment components, dipole orientations, volumes, surface areas and the lengths and charges of their C-termini. The results of our computatations in this regard are shown in Table 2.” The Table 1 below contains the data presented in the Table 2 of the article.

TABLE 1

| | | | | | Net | Volume | Area | |
|---|---|---|---|---|---|---|---|---|
| Name | <M_x> | <M_y> | <M_z> | <IMI> | Charge | A\hat{ }3 | A\hat{ }2 | |
| TBA1_ANEPH | −3.02E+002 | −6.06E+002 | 1.16E+003 | 1.34E+003 | −22 | 43722.51 | 46119.66 | |
| TBA1_ARATH | 5.03E+001 | −4.69E+002 | 1.50E+003 | 1.57E+003 | −24 | 43725.6 | 46097.33 | |
| TBA1_CHICK | −2.84E+002 | −9.75E+002 | 1.61E+003 | 1.90E+003 | −21 | 40489.52 | 43082.05 | F |
| TBA1_CHLRE | −6.10E+001 | −7.44E+002 | 7.28E+002 | 1.04E+003 | −21 | 43642.98 | 45933.57 | |
| TBA1_DROME | 5.95E+001 | −6.29E+002 | 1.05E+003 | 1.23E+003 | −22 | 44030.65 | 46824.19 | |
| TBA1_ELEIN | −5.54E+001 | −3.29E+002 | 1.37E+003 | 1.41E+003 | −24 | 43860.52 | 46749.02 | |
| TBA1 _EMENI | −1.86E+002 | −1.23E+003 | 7.71E+002 | 1.47E+003 | −24 | 44069.69 | 46434.2 | |
| TBA1 _ENTHI | 2.50E+002 | −6.70E+002 | 1.46E+002 | 7.30E+002 | −10 | 44061.3 | 46460.88 | |
| TBA1_HOMAM | −1.53E+002 | −1.15E+003 | 9.52E+002 | 1.50E+003 | −22 | 44167.33 | 46824.48 | |
| TBA1_HORVU | 1.55E+002 | −3.40E+002 | 1.27E+003 | 1.32E+003 | −23 | 43590.96 | 45826.84 | |
| TBA1 _HUMAN | −4.67E+002 | −8.10E+002 | 1.11E+003 | 1.45E+003 | −24 | 44250.31 | 47173.96 | |
| TBA1_MAIZE | 1.03E+002 | −3.28E+002 | 1.28E+003 | 1.32E+003 | −24 | 43834.72 | 46651.62 | |
| TBA1_MOUSE | −3.33E+002 | −1.21E+003 | 7.70E+002 | 1.47E+003 | −24 | 44263.22 | 47101.9 | |
| TBA1_NEUCR | 4.87E+001 | −6.76E+002 | 6.94E+002 | 9.70E+002 | −19 | 44052.23 | 46358.29 | |
| TBA1_ORYSA | −2.19E+002 | −1.16E+003 | 1.12E+003 | 1.62E+003 | −24 | 43648.39 | 45939.87 | |
| TBA1_PARLI | 2.71E+002 | −1.19E+003 | 1.78E+003 | 2.16E+003 | −25 | 44183.57 | 46803.97 | |
| TBA1_PEA | −3.23E+002 | −7.69E+002 | 1.05E+003 | 1.34E+003 | −23 | 43567.64 | 45723.58 | |
| TBA1_PELFA | −4.01E+002 | −1.41E+003 | 8.27E+002 | 1.68E+003 | −24 | 43906.79 | 46567.68 | |
| TBA1_PNECA | −2.57E+001 | −9.24E+002 | 9.87E+002 | 1.35E+003 | −20 | 44334.85 | 47012.18 | |
| TBA1_SCHPO | −2.56E+000 | −1.26E+003 | 6.43E+002 | 1.41E+003 | −22 | 44895.34 | 47968.48 | |
| TBA1_STYLE | −2.03E+002 | −1.27E+003 | 8.29E+002 | 1.53E+003 | −23 | 43243.03 | 45451.26 | |
| TBA1_VOLCA | −1.26E+002 | −8.00E+002 | 6.88E+002 | 1.06E+003 | −21 | 43630.21 | 45981.34 | |
| TBA1_YEAST | −1.90E+002 | −9.79E+002 | 4.23E+002 | 1.08E+003 | −22 | 43873.76 | 46461.59 | |
| TBA2_ANEPH | −2.78E+002 | −8.85E+002 | 1.35E+003 | 1.64E+003 | −15 | 35461.49 | 37487.42 | F |
| TBA2_ARATH | −1.18E+002 | −6.40E+002 | 1.50E+003 | 1.63E+003 | −23 | 43766.11 | 46803.45 | |
| TBA2_CAEEL | −1.39E+002 | −8.51E+002 | 1.07E+003 | 1.37E+003 | −22 | 43890.89 | 46319.2 | |
| TBA2_CHICK | −9.83E+001 | −2.00E+002 | 1.12E+003 | 1.14E+003 | −25 | 43774.22 | 46365.41 | |
| TBA2_CHLRE | −1.41E+002 | −8.09E+002 | 7.99E+002 | 1.15E+003 | −22 | 43601.27 | 45660.58 | |
| TBA2_DROME | −9.25E+001 | −1.09E+003 | 7.03E+002 | 1.30E+003 | −21 | 44116.52 | 46892.4 | |
| TBA2_ELEIN | 3.81E+001 | −3.80E+002 | 1.39E+003 | 1.44E+003 | −21 | 43843.11 | 45940.56 | |
| TBA2_EM EN I | −3.11E+002 | −1.41E+003 | 6.14E+002 | 1.57E+003 | −21 | 44173.08 | 46890.29 | |
| TBA2_HOMAM | −7.38E+002 | −6.68E+002 | 9.66E+002 | 1.39E+003 | −20 | 44252.35 | 47078.27 | |
| TBA2_HORVU | −1.24E+002 | −5.45E+002 | 1.44E+003 | 1.54E+003 | −24 | 43705.55 | 46254.23 | |
| TBA2_HUMAN | −7.89E+001 | −1.27E+003 | 7.92E+002 | 1.49E+003 | −23 | 44045.61 | 46631.11 | |
| TBA2_MAIZE | 3.87E+001 | −3.08E+002 | 1.32E+003 | 1.35E+003 | −24 | 43670.06 | 46059.53 | |
| TBA2_MOUSE | −4.62E+002 | −1.26E+003 | 7.32E+002 | 1.53E+003 | −24 | 44188.6 | 46902.07 | |
| TBA2_NEUCR | −4.64E+002 | −8.59E+002 | 6.78E+002 | 1.19E+003 | −22 | 43969.77 | 46397.94 | |
| TBA2PATVU | −7.08E+002 | −1.23E+003 | 9.86E+002 | 1.73E+003 | −24 | 44205.67 | 46802.41 | |
| TBA2_PELFA | −5.63E+002 | −1.35E+003 | 1.09E+003 | 1.82E+003 | −25 | 43972.36 | 46729.96 | |
| TBA2_SCHPO | −3.69E+002 | −6.06E+002 | 7.84E+002 | 1.06E+003 | −23 | 44413.68 | 47084.43 | |
| TBA2_STYLE | −1.52E+002 | −1.20E+003 | 1.42E+003 | 1.87E+003 | −21 | 43462.96 | 45794.68 | |
| TBA3_ARATH | −1.37E+002 | −6.23E+002 | 1.31E+003 | 1.45E+003 | −23 | 43767.64 | 46340.56 | |
| TBA3_CHICK | 9.52E+001 | −1.35E+003 | 4.35E+002 | 1.42E+003 | −11 | 31862.21 | 34076.89 | F |
| TBA3_DROME | 8.39E+001 | −5.89E+002 | 9.56E+002 | 1.13E+003 | −22 | 44025.38 | 46744.36 | |
| TBA3_ELEIN | −2.23E+002 | −1.06E+003 | 7.94E+002 | 1.34E+003 | −24 | 43622.68 | 45927.05 | |
| TBA3_HOMAM | −4.66E+002 | −1.35E+003 | 9.96E+002 | 1.74E+003 | −24 | 44023.88 | 46424.8 | |
| TBA3_HORVU | 1.67E+002 | −2.61E+002 | 1.19E+003 | 1.23E+003 | −24 | 43774.25 | 46614.74 | |
| TBA3_MAIZE | −2.26E+002 | −9.73E+002 | 1.25E+003 | 1.60E+003 | −20 | 43523.21 | 45861.11 | |
| TBA3_MOUSE | −7.89E+001 | −1.27E+003 | 7.92E+002 | 1.49E+003 | −23 | 44045.61 | 46631.11 | |
| TBA3_YEAST | −3.29E+001 | −1.38E+003 | 7.81E+001 | 1.38E+003 | −20 | 43772.88 | 46394.31 | |
| TBA4_CHICK | −7.55E+001 | −1.23E+003 | 1.34E+003 | 1.82E+003 | −19 | 31763.1 | 34085.01 | F |
| TBA4_DROME | −4.56E+002 | −9.92E+002 | 8.14E+002 | 1.36E+003 | −18 | 44749.62 | 46802.21 | |
| TBA4_HUMAN | −4.56E+001 | −7.37E+002 | 1.29E+003 | 1.49E+003 | −24 | 44006.12 | 46802.17 | |
| TBA4_MAIZE | 1.91E+002 | 5.47E+002 | 5.31E+002 | 7.86E+002 | −13 | 5653.1 | 6441.79 | F |
| TBA5_CHICK | −5.61E+002 | −8.51E+002 | 9.93E+002 | 1.42E+003 | −24 | 44001.41 | 46787.46 | |
| TBA5_MAIZE | 1.18E+002 | −3.59E+002 | 1.20E+003 | 1.26E+003 | −24 | 43664.32 | 46180.91 | |
| TBA6_ARATH | −4.74E+002 | −9.38E+002 | 1.03E+003 | 1.47E+003 | −23 | 43549.12 | 45981.06 | |
| TBA6_HUMAN | −1.51E+002 | −8.12E+002 | 9.12E+002 | 1.23E+003 | −23 | 44019.72 | 46935.74 | |
| TBA6_MAIZE | 1.05E+002 | −2.29E+002 | 1.28E+003 | 1.30E+003 | −24 | 43616.26 | 45962.24 | |
| TBA6_MOUSE | −4.97E+002 | −8.04E+002 | 8.36E+002 | 1.26E+003 | −23 | 44005.43 | 46878.15 | |
| TBA8_CAEEL | 4.38E+001 | −1.35E+003 | 6.07E+002 | 1.48E+003 | −21 | 44092.19 | 46452.13 | |
| TBA8_CHICK | −3.14E+002 | −1.21E+003 | 6.74E+002 | 1.42E+003 | −17 | 31941.5 | 34147.91 | F |
| TBA8_HUMAN | −2.56E+002 | −1.13E+003 | 6.47E+002 | 1.33E+003 | −24 | 44108.74 | 46846.78 | |
| TBA8_MOUSE | 2.58E+001 | −9.76E+002 | 5.25E+002 | 1.11E+003 | −23 | 44094.24 | 46772.18 | |
| TBA_AJECA | 4.11E+002 | −5.71E+002 | −3.80E+002 | 8.00E+002 | −11 | 40915.67 | 42810.21 | F |
| TBAA_PN ECA | 4.02E+002 | −4.58E+002 | −3.47E+002 | 7.01E+002 | 0 | 21163.74 | 22925.51 | F |
| TBAA_SCHCO | 6.78E+000 | −9.52E+002 | 6.63E+002 | 1.16E+003 | −20 | 43528.88 | 46457.95 | |
| TBA_AVESA | 4.40E+002 | −3.62E+002 | 3.57E+002 | 6.72E+002 | −17 | 43193.08 | 45318.02 | |
| TBA_BLEJA | −1.14E+002 | 4.91E+001 | 5.37E+001 | 1.35E+002 | −17 | 4939.05 | 5726.72 | F |
| TBA_BOMMO | −1.56E+002 | −1.02E+003 | 5.91E+002 | 1.19E+003 | −23 | 44002.66 | 46587.95 | |
| TBAB_SCHCO | 1.68E+002 | −8.87E+002 | 1.06E+003 | 1.39E+003 | −17 | 43480.44 | 46447.1 | |
| TBA_CANAL | −2.94E+002 | −1.58E+003 | 1.45E+002 | 1.61E+003 | −20 | 43827.47 | 46383.14 | |
| TBA_CHLVU | −3.40E+002 | −1.04E+003 | 6.60E+002 | 1.28E+003 | −23 | 43800.27 | 46511.59 | |
| TBA_DICDI | −2.65E+002 | −8.18E+002 | 4.88E+002 | 9.88E+002 | −15 | 44897.67 | 47487.03 | |
| TBAD_PHYPO | 7.24E+001 | −8.54E+002 | 1.25E+003 | 1.51E+003 | −22 | 43832.96 | 46203.15 | |
| TBAE_PHYPO | 5.38E+001 | −8.04E+002 | 9.48E+002 | 1.24E+003 | −22 | 43712.79 | 46164.96 | |
| TBA_EUGGR | −5.50E+002 | −9.02E+002 | 7.55E+002 | 1.30E+003 | −23 | 44007.88 | 46521.52 | |
| TBA_EU POC | 3.58E+000 | −8.90E+002 | 8.98E+002 | 1.26E+003 | −22 | 43646.63 | 46268.82 | |
| TBA_EU PVA | −3.61E+002 | −9.45E+002 | 6.25E+002 | 1.19E+003 | −22 | 43678.31 | 46191.85 | |
| TBA_HAECO | −5.01E+002 | −9.24E+002 | 1.01E+003 | 1.45E+003 | −23 | 44184.78 | 46867.6 | |
| TBA_LEPSE | 0 | | | | | 2420.34 | 2750.51 | F |
| TBA_LYTPI | −8.32E+002 | −1.07E+003 | 1.57E+003 | 2.07E+003 | −11 | 15959.86 | 17858.74 | |
| TBA_MYCGR | 1.31E+001 | −1.13E+003 | 8.25E+001 | 1.13E+003 | −24 | 43927.86 | 46753.33 | |
| TBA_NAEGR | −4.44E+002 | −1.04E+003 | 3.75E+002 | 1.19E+003 | −23 | 44031.56 | 47036.09 | |
| TBA_NOTVI | −1.47E+002 | −8.20E+002 | 1.11E+003 | 1.39E+003 | −24 | 44167.23 | 47197.14 | |
| TBAN_PHYPO | −1.15E+002 | −9.60E+002 | 8.97E+002 | 1.32E+003 | −23 | 43607.45 | 45977.08 | |
| TBA_OCTDO | −1.92E+002 | −1.38E+003 | 1.19E+003 | 1.84E+003 | −22 | 44189.74 | 46624.65 | |
| TBA_OCTDU | −3.40E+002 | −1.21E+003 | 1.28E+003 | 1.79E+003 | −12 | 23897.38 | 25881.13 | F |
| TBA_ONCKE | −1.99E+002 | −1.15E+003 | 1.11E+003 | 1.61E+003 | −24 | 43491.82 | 46581.51 | |
| TBA_OXYGA | −8.66E+001 | −1.08E+003 | 8.99E+002 | 1.41E+003 | −23 | 43713.34 | 46373.82 | |
| TBA_PICAB | −1.02E+002 | −9.19E+001 | −1.23E+002 | 1.84E+002 | −10 | 11088.01 | 12137.53 | F |
| TBA_PIG | −4.06E+002 | −1.20E+003 | 6.32E+002 | 1.42E+003 | −25 | 44083.31 | 46762.84 | |
| TBA_PLAFK | −6.63E+002 | −1.02E+003 | 1.09E+003 | 1.64E+003 | −22 | 44159.95 | 46868.83 | |
| TBA_PLAYO | −4.57E+002 | −9.08E+002 | 9.83E+002 | 1.41E+003 | −12 | 19399.72 | 20787.14 | F |
| TBA_PRUDU | −2.93E+002 | −1.09E+003 | 7.65E+002 | 1.36E+003 | −23 | 43611.95 | 46257.89 | |
| TBA_SORMA | −5.77E+001 | −5.78E+002 | 8.84E+002 | 1.06E+003 | −23 | 43781.31 | 46691.13 | |
| TBA_TETPY | 1.49E+002 | −8.36E+002 | 8.32E+002 | 1.19E+003 | −21 | 43728.45 | 46142.49 | |
| TBA_TETTH | −5.13E+001 | −7.26E+002 | 8.46E+002 | 1.12E+003 | −21 | 43757.64 | 46334.88 | |
| TBAT_ONCMY | −1.81E+002 | −1.07E+003 | 8.98E+002 | 1.41E+003 | −23 | 44043.36 | 46640.52 | |
| TBA_TO R MA | −2.02E+002 | −1.15E+003 | 6.45E+002 | 1.33E+003 | −24 | 44318.53 | 47358.43 | |
| TBA_TOXGO | 2.03E+002 | −1.08E+003 | 1.11E+003 | 1.56E+003 | −23 | 44098.44 | 46708.74 | |
| TBATRYBR | −1.72E+002 | −1.00E+003 | 8.63E+002 | 1.33E+003 | −24 | 43867.8 | 46476.58 | |
| TBA_TRYCR | −3.14E+002 | −1.05E+003 | 9.14E+002 | 1.42E+003 | −25 | 43758.17 | 46172.03 | |
| TBA_WHEAT | 2.00E+002 | −6.80E+002 | 1.39E+003 | 1.56E+003 | −24 | 43805.34 | 46562.2 | |
| TBA_XEN LA | −2.31E+002 | −1.10E+003 | 6.83E+002 | 1.31E+003 | −23 | 43943 | 46478.64 | |
| TBB1_ANEPH | −2.40E+002 | −6.68E+002 | 1.53E+003 | 1.69E+003 | −21 | 43331.36 | 45949.82 | F |
| TBB1_ARATH | −1.22E+003 | −1.02E+003 | 2.69E+003 | 3.13E+003 | −27 | 43751.93 | 46146.83 | |
| TBB1_AVESA | −8.00E+002 | −1.71E+003 | 2.56E+003 | 3.18E+003 | −25 | 38101.3 | 41156.74 | |
| TBB1_BRUPA | −2.70E+002 | −6.98E+002 | 1.81E+003 | 1.96E+003 | −26 | 43981.4 | 46705.33 | |
| TBB1 _CHICK | −1.13E+003 | −1.02E+003 | 1.51E+003 | 2.15E+003 | −25 | 43815.13 | 46865.04 | |
| TBB1 _CHOCK | −6.25E+002 | 2.36E+002 | 1.77E+003 | 1.89E+003 | −27 | 43977.7 | 45918.5 | |
| TBB1_COLGL | −1.39E+003 | −1.22E+003 | 3.07E+003 | 3.58E+003 | −24 | 43616.55 | 45527.47 | |
| TBB1_COLGR | −2.58E+002 | −6.84E+002 | 2.23E+003 | 2.35E+003 | −24 | 43341.08 | 45417.82 | |
| TBB1_CYAPA | −1.03E+003 | −1.03E+003 | 1.46E+003 | 2.06E+003 | −25 | 43703.53 | 46639.47 | |
| TBB1_DAUCA | −1.29E+003 | −4.57E+002 | 2.76E+003 | 3.08E+003 | −17 | 31337.94 | 33360.35 | |
| TBB1_ELEIN | −1.10E+003 | −1.03E+003 | 2.71E+003 | 3.10E+003 | −26 | 43749.89 | 46609.62 | |
| TBB1 _EMENI | −3.24E+002 | −1.74E+003 | 1.74E+003 | 2.48E+003 | −23 | 43750.84 | 46675.24 | |
| TBB1 _GADMO | −1.02E+003 | −1.16E+003 | 1.20E+003 | 1.95E+003 | −25 | 43817.93 | 47122.12 | |
| TBB1_GEOCN | −9.55E+002 | −9.87E+002 | 1.33E+003 | 1.91E+003 | −24 | 43808.6 | 46274.2 | |
| TBB1_HOMAM | −1.24E+003 | −1.24E+003 | 2.66E+003 | 3.19E+003 | −24 | 44266.15 | 45948.21 | |
| TBB1 _HU MAN | −4.95E+002 | −1.36E+003 | 2.04E+003 | 2.50E+003 | −25 | 43765.02 | 46853.55 | |
| TBB1 _LU PAL | −1.56E+003 | −1.20E+003 | 2.93E+003 | 3.53E+003 | −25 | 43898.24 | 46734.22 | |
| TBB1 _MAIZE | −8.98E+002 | −1.44E+003 | 2.28E+003 | 2.84E+003 | −25 | 43776.83 | 46781.39 | |
| TBB1 _MANSE | 3.26E+001 | −4.73E+002 | 1.77E+003 | 1.83E+003 | −25 | 44083.08 | 46838.17 | |
| TBB1_NOTCO | −9.76E+002 | −1.32E+003 | 2.51E+003 | 3.00E+003 | −25 | 43698.37 | 46442.69 | |
| TBB1 _ORYSA | −1.04E+003 | −1.14E+003 | 1.59E+003 | 2.22E+003 | −25 | 43757.44 | 46832.09 | |
| TBB1 _PARTE | −1.64E+002 | −1.30E+003 | 1.62E+003 | 2.08E+003 | −24 | 43491.13 | 46266.33 | |
| TBB1_PEA | −1.68E+003 | −1.21E+003 | 3.14E+003 | 3.76E+003 | −26 | 44208.97 | 46988.05 | F |
| TBB1_PHYPO | −2.55E+002 | −9.30E+002 | 1.51E+003 | 1.79E+003 | −23 | | | |
| TBB1 _PORPU | −9.28E+002 | −9.18E+002 | 2.05E+003 | 2.43E+003 | −28 | 43887.44 | 47046.49 | F |
| TBB1 _RAT | −1.24E+003 | −1.25E+003 | 2.47E+003 | 3.04E+003 | −25 | 43855.58 | 46823.88 | |
| TBB1_SOLTU | −1.31E+003 | −1.08E+003 | 3.00E+003 | 3.45E+003 | −26 | 43921.08 | 45964.17 | |
| TBB1_SOYBN | −1.99E+002 | −1.02E+003 | 1.77E+003 | 2.06E+003 | −22 | 43716.86 | 46392.04 | |
| TBB1 _TRIVI | −2.09E+002 | −1.21E+003 | 1.46E+003 | 1.91E+003 | −21 | 43239.19 | 45386.6 | |
| TBB1_VOLCA | −5.67E+002 | −1.31E+003 | 1.89E+003 | 2.37E+003 | −24 | 43622.7 | 46596.3 | |
| TBB1_WHEAT | −8.62E+002 | −8.33E+002 | 2.00E+003 | 2.33E+003 | −25 | 44053.19 | 47377.04 | |
| TBB2_ANEPH | −2.34E+002 | −1.01E+003 | 1.48E+003 | 1.81E+003 | −18 | 40451.27 | 43579.53 | |
| TBB2_ARATH | −1.86E+003 | −8.40E+002 | 3.47E+003 | 4.03E+003 | −27 | 44380.25 | 47532.89 | |
| TBB2_CAEEL | −1.22E+003 | −1.30E+003 | 2.60E+003 | 3.15E+003 | −24 | 44042.41 | 46516.26 | |
| TBB2_CHICK | −9.85E+002 | −1.18E+003 | 2.51E+003 | 2.94E+003 | −24 | 43790.78 | 46641.57 | |
| TBB2_COLGL | 4.51E+001 | −1.25E+003 | 2.44E+003 | 2.74E+003 | −24 | 43772.28 | 46801.36 | |
| TBB2_COLG R | −6.48E+002 | −1.73E+003 | 2.32E+003 | 2.96E+003 | −24 | 43776.26 | 46725.65 | |
| TBB2DAUCA | −3.80E+002 | −1.04E+003 | 1.48E+003 | 1.85E+003 | −25 | 43469.26 | 46734.07 | |
| TBB2_DROER | −1.53E+003 | −1.19E+003 | 3.02E+003 | 3.59E+003 | −25 | 43757.19 | 46469.02 | |
| TBB2_DROM E | −1.08E+003 | −1.15E+003 | 2.59E+003 | 3.03E+003 | −26 | 43646.93 | 46257.22 | |
| TBB2_ELEIN | −5.42E+002 | −6.38E+002 | 2.37E+003 | 2.52E+003 | −26 | 44115.31 | 47287.17 | |
| TBB2_EMENI | −3.49E+002 | −1.26E+003 | 2.11E+003 | 2.48E+003 | −22 | 43740.18 | 46549.31 | |
| TBB2_ERYPI | −1.03E+003 | −1.43E+003 | 1.94E+003 | 2.62E+003 | −22 | 43844.47 | 46799.54 | |
| TBB2_G EOCN | −1.16E+003 | −5.41E+002 | 2.71E+003 | 3.00E+003 | −28 | 44192.98 | 46317.34 | |
| TBB2_HOMAM | −4.43E+002 | −9.20E+001 | 2.03E+003 | 2.08E+003 | −24 | 44467.04 | 45943.42 | |
| TBB2_HUMAN | −1.83E+002 | −1.53E+003 | 1.72E+003 | 2.31E+003 | −25 | 43874.46 | 47063.85 | |
| TBB2_LUPAL | −1.68E+003 | −1.16E+003 | 3.46E+003 | 4.02E+003 | −26 | 44006.64 | 46759.35 | |
| TBB2_MAIZE | −9.72E+002 | −1.25E+003 | 2.49E+003 | 2.95E+003 | −23 | 43627.92 | 46573.3 | |
| TBB2_ORYSA | −7.82E+002 | −1.02E+003 | 1.61E+003 | 2.06E+003 | −25 | 44025.13 | 47076.73 | |
| TBB2_PEA | −1.87E+003 | −1.43E+003 | 2.80E+003 | 3.66E+003 | −28 | 44119.64 | 47264.21 | |
| TBB2_1³ HYPO | −1.62E+003 | −9.87E+002 | 3.29E+003 | 3.80E+003 | −24 | 44197.53 | 47050.16 | |
| TBB2_PORPU | −8.84E+002 | −5.18E+002 | 1.80E+003 | 2.07E+003 | −27 | 41546.31 | 44676.99 | |
| TBB2_SOLTU | −9.18E+002 | −1.31E+003 | 2.27E+003 | 2.78E+003 | −26 | 44046.81 | 46135.05 | |
| TBB2_SOYBN | −1.21E+003 | −1.42E+003 | 2.75E+003 | 3.32E+003 | −26 | 44355.5 | 47559.1 | |
| TBB2_TRIVI | −5.10E+002 | −9.99E+002 | 2.41E+003 | 2.66E+003 | −24 | 43739.12 | 46059.81 | |
| TBB2_WH EAT | −1.29E+003 | −8.96E+002 | 3.24E+003 | 3.61E+003 | −27 | 43864.6 | 46565.28 | |
| TBB2_XENLA | −8.81E+002 | −8.68E+002 | 1.96E+003 | 2.32E+003 | −24 | 43639.84 | 46526.04 | |
| TBB3_ANEPH | −7.63E+002 | −8.82E+002 | 1.87E+003 | 2.20E+003 | −9 | 24028.31 | 25945.03 | F |
| TBB3_CHICK | −1.48E+003 | −1.08E+003 | 3.01E+003 | 3.52E+003 | −26 | 43756.1 | 46490.85 | |
| TBB3_D ROME | −1.29E+003 | −1.65E+003 | 2.45E+003 | 3.22E+003 | −23 | 44396.18 | 46320.02 | |
| TBB3_ELEIN | −1.51E+003 | −1.19E+003 | 2.31E+003 | 3.00E+003 | −27 | 43974.3 | 47141.63 | |
| TBB3_MAIZE | −1.40E+003 | −1.05E+003 | 2.84E+003 | 3.34E+003 | −25 | 43485.86 | 46040.71 | |
| TBB3_ORYSA | −1.39E+003 | −9.58E+002 | 2.79E+003 | 3.26E+003 | −27 | 43797.24 | 46373.71 | |
| TBB3_PEA | −1.46E+003 | −1.53E+003 | 2.81E+003 | 3.52E+003 | −27 | 43323.16 | 46648.94 | F |
| TBB3_FORPU | −1.17E+003 | −1.14E+003 | 2.60E+003 | 3.07E+003 | −26 | 43529.91 | 46185.14 | |
| TBB3_SOYBN | 4.79E+002 | −1.01E+003 | −2.15E+002 | 1.14E+003 | −9 | 40339.02 | 43199.08 | F |
| TBB3_WHEAT | −1.42E+003 | −1.03E+003 | 3.02E+003 | 3.49E+003 | −28 | 43670.95 | 46343.03 | |
| TBB4_ARATH | −1.06E+003 | −1.21E+003 | 2.38E+003 | 2.87E+003 | −25 | 43750.97 | 46535.59 | |
| TBB4_CAEEL | −1.01E+003 | −1.29E+003 | 1.88E+003 | 2.49E+003 | −24 | 43683.61 | 46649.3 | |
| TBB4_CHICK | −1.14E+003 | −1.37E+003 | 2.71E+003 | 3.24E+003 | −24 | 44048.85 | 46490.78 | |
| TBB4_ELEI N | −1.14E+003 | −9.75E+002 | 2.27E+003 | 2.72E+003 | −25 | 43906.03 | 46993.99 | |
| TBB4_HUMAN | −1.15E+003 | −8.25E+002 | 2.06E+003 | 2.49E+003 | −25 | 44223.15 | 47073.77 | |
| TBB4_MAIZE | −1.01E+003 | −9.45E+002 | 2.18E+003 | 2.58E+003 | −24 | 43757.47 | 46283.88 | |
| TBB4_PORPU | −1.71E+003 | −1.27E+003 | 2.68E+003 | 3.42E+003 | −28 | 44129.26 | 47186.23 | |
| TBB4_W H EAT | −7.03E+002 | −1.24E+003 | 2.34E+003 | 2.75E+003 | −25 | 43821.6 | 47046.07 | |
| TBB4_XENLA | −1.18E+003 | −1.11E+003 | 2.71E+003 | 3.16E+003 | −24 | 43722.48 | 46674.76 | |
| TBB5_ARATH | −1.80E+003 | −1.08E+003 | 3.25E+003 | 3.86E+003 | −28 | 44001.8 | 46634.56 | |
| TBB5_CHICK | −7.93E+002 | −1.16E+003 | 2.21E+003 | 2.62E+003 | −25 | 43891.79 | 46604.44 | |
| TBB5_ECTVR | −1.27E+003 | −1.16E+003 | 2.72E+003 | 3.22E+003 | −25 | 43750.92 | 46441.18 | |
| TBB5_HUMAN | −8.71E+002 | −1.12E+003 | 1.95E+003 | 2.41E+003 | −24 | 43580.58 | 46339.09 | |
| TBB5_MAIZE | −1.23E+003 | −1.21E+003 | 2.47E+003 | 3.01E+003 | −24 | 43798.93 | 46550.2 | |
| TBB5_WHEAT | −7.11E+002 | −7.94E+002 | 2.47E+003 | 2.69E+003 | −26 | 44109.94 | 47148.65 | |
| TBB6_ARATH | −1.78E+003 | −1.24E+003 | 2.49E+003 | 3.30E+003 | −28 | 44352.88 | 47605.4 | |
| TBB6_CHICK | −1.10E+001 | −1.14E+003 | 1.81E+003 | 2.14E+003 | −20 | 44013.78 | 46378.76 | |
| TBB6_ECTVR | −1.19E+003 | −1.20E+003 | 2.80E+003 | 3.27E+003 | −24 | 43894.94 | 46566.79 | |
| TBB6_MAIZE | −8.83E+002 | −1.00E+003 | 1.53E+003 | 2.03E+003 | −25 | 44054.31 | 47248.64 | |
| TBB7_ARATH | −1.53E+003 | −1.27E+003 | 3.15E+003 | 3.72E+003 | −26 | 44339.38 | 47096.68 | |
| TBB7_CAEBR | −2.49E+002 | −1.05E+003 | 1.53E+003 | 1.87E+003 | −21 | 43204.36 | 45682.33 | |
| TBB7_CAEEL | −1.65E+002 | −9.89E+002 | 1.48E+003 | 1.79E+003 | −19 | 43248.82 | 45913.33 | |
| TBB7_CHICK | −1.07E+003 | −1.20E+003 | 2.50E+003 | 2.97E+003 | −24 | 43586.17 | 46372.26 | |
| TBB7_MAIZE | −1.62E+003 | −1.02E+003 | 3.29E+003 | 3.81E+003 | −28 | 43810.2 | 46505.18 | |
| TBB8_ARATH | −1.44E+003 | −1.09E+003 | 3.25E+003 | 3.72E+003 | −25 | 44295.12 | 47021.18 | |
| TBB8_MAIZE | −2.88E+002 | −1.30E+003 | 1.98E+003 | 2.39E+003 | −25 | 43821.89 | 47069.82 | |
| TBB9_ARATH | −1.85E+002 | −1.19E+003 | 1.87E+003 | 2.22E+003 | −27 | 43541.37 | 46710.21 | |
| TBB_ACHKL | −1.49E+003 | −1.18E+003 | 2.78E+003 | 3.37E+003 | −27 | 43582.08 | 46093.01 | |
| TBB_ACRCO | −2.96E+002 | −1.68E+003 | 2.83E+003 | 3.31E+003 | −25 | 44030.34 | 47131.02 | |
| TBB_AJECA | −1.90E+001 | −1.42E+003 | 1.43E+003 | 2.02E+003 | −17 | 43899.95 | 46111.64 | |
| TBB_ASPFL | −1.25E+003 | −1.07E+003 | 3.51E+003 | 3.88E+003 | −23 | 43818.01 | 46347.28 | |
| TBB_ASPPA | −1.09E+003 | −1.17E+003 | 3.41E+003 | 3.77E+003 | −23 | 43974.21 | 46807.66 | |
| TBB_BABBO | −7.24E+001 | −9.18E+002 | 9.11E+002 | 1.30E+003 | −22 | 43389.67 | 46335.4 | |
| TBB_BOMMO | −1.75E+002 | −1.61E+003 | 3.52E+003 | 3.87E+003 | −25 | 44236.29 | 47190.27 | |
| TBB_BOTCI | −8.12E+001 | −1.52E+003 | 2.48E+003 | 2.91E+003 | −22 | 43733.8 | 46687 | |
| TBB_CANAL | −7.66E+002 | −1.32E+003 | 2.58E+003 | 3.00E+003 | −27 | 43649.84 | 45896.27 | |
| TBB_CEPAC | −6.54E+002 | −1.75E+003 | 2.46E+003 | 3.09E+003 | −24 | 43755.92 | 46590.03 | |
| TBB_CHLIN | −8.12E+002 | −1.08E+003 | 2.18E+003 | 2.56E+003 | −24 | 43440.63 | 46047.48 | |
| TBB_CHLRE | −7.63E+002 | −1.04E+003 | 1.67E+003 | 2.11E+003 | −24 | 43580.97 | 46513.44 | |
| TBB_CICAR | −1.47E+003 | −1.26E+003 | 2.76E+003 | 3.37E+003 | −26 | 44093.85 | 46346.37 | |
| TBB_DICDI | −2.34E+002 | −3.61E+002 | 2.34E+003 | 2.38E+003 | −25 | 44756.23 | 46368.54 | |
| TBB_EIMTE | −8.00E+002 | −1.16E+003 | 2.38E+003 | 2.76E+003 | −24 | 43849.31 | 46691.1 | |
| TBB_EPITY | −1.72E+002 | −9.30E+002 | 2.54E+003 | 2.71E+003 | −24 | 43981.99 | 47087.79 | |
| TBB_ERYGR | −1.11E+003 | −1.62E+003 | 2.49E+003 | 3.17E+003 | −21 | 43521.09 | 46022.36 | |
| TBB_EUGGR | −5.27E+002 | −9.96E+002 | 1.74E+003 | 2.08E+003 | −28 | 43338.8 | 45940.86 | |
| TBB_EUPCR | −1.29E+003 | −1.16E+003 | 2.41E+003 | 2.97E+003 | −26 | 43733.12 | 46318.45 | |
| TBB_EUPFO | −3.46E+002 | −9.23E+002 | 2.17E+003 | 2.38E+003 | −23 | 43736.75 | 46621.81 | |
| TBB_EUPOC | −1.09E+003 | −1.05E+003 | 2.34E+003 | 2.79E+003 | −25 | 43474.13 | 46098.76 | |
| TBB_G!ALA | −1.11E+003 | −9.79E+002 | 2.43E+003 | 2.85E+003 | −24 | 43973.16 | 47134.22 | |
| TBB_GIBFU | −1.09E+003 | −1.15E+003 | 3.42E+003 | 3.76E+003 | −24 | 43717.6 | 46450.54 | |
| TBB_HALDI | 2.70E+002 | −1.27E+003 | 5.88E+002 | 1.43E+003 | −5 | 33789.64 | 36441.03 | F |
| TBB_HORVU | −1.62E+003 | −1.07E+003 | 3.28E+003 | 3.82E+003 | −27 | 43828.44 | 46711.06 | |
| TBB_LEI ME | −4.57E+002 | −1.16E+003 | 1.79E+003 | 2.18E+003 | −25 | 43640.51 | 46103.21 | |
| TBB_LYMST | −4.11E+002 | 9.15E+001 | 1.44E+003 | 1.50E+003 | −16 | 11210.57 | 12653.09 | F |
| TBB_LYTPI | −1.31E+003 | −6.00E+002 | 3.00E+003 | 3.33E+003 | −13 | 17813.1 | 19768.08 | F |
| TBB_MYCPJ | −1.06E+003 | −1.44E+003 | 3.06E+003 | 3.54E+003 | −22 | 43590.41 | 46322.14 | |
| TBB_NAEGR | −9.11E+002 | −1.50E+003 | 2.96E+003 | 3.44E+003 | −26 | 44385.1 | 47524.01 | |
| TBB_NEUCR | −8.67E+002 | −1.30E+003 | 3.63E+003 | 3.95E+003 | −24 | 43678.94 | 46401.01 | |
| TBB_OCTDO | −7.15E+002 | −5.08E+002 | 1.86E+003 | 2.06E+003 | −23 | 44106.16 | 46618.28 | |
| TBB_ONCGI | −7.02E+002 | −1.07E+003 | 2.06E+003 | 2.42E+003 | −21 | 43865.63 | 46450.72 | |
| TBB_PAALI | −1.51E+003 | −1.20E+003 | 3.26E+003 | 3.78E+003 | −26 | 43883.1 | 46679.47 | |
| TBB_PENDI | −4.37E+002 | −1.44E+003 | 2.26E+003 | 2.71E+003 | −21 | 43814.64 | 46891.07 | |
| TBB_PESMI | −3.95E+002 | −1.50E+003 | 2.53E+003 | 2.97E+003 | −24 | 43722.64 | 46550.51 | |
| TBB_P NANO | −7.61E+002 | −1.12E+003 | 2.67E+003 | 2.99E+003 | −21 | 43777.75 | 46470 | |
| TBB_PHYCI | −6.90E+002 | −1.01E+003 | 1.92E+003 | 2.27E+003 | −24 | 43659.2 | 46213.31 | |
| TBB_PIG | −3.59E+002 | −1.55E+003 | 1.91E+003 | 2.48E+003 | −25 | 43854.42 | 47042.18 | |
| TBB_PLASA | −6.56E+002 | −1.43E+003 | 1.55E+003 | 2.21E+003 | −28 | 43731.91 | 46620.07 | |
| TBB_POAFK | −1.06E+003 | −1.24E+003 | 1.40E+003 | 2.14E+003 | −27 | 43684.84 | 46607.02 | |
| TBB_PLESA | −1.51E+003 | −1.06E+003 | 2.51E+003 | 3.12E+003 | −25 | 44047.21 | 46981.21 | |
| TBB_PNECA | 5.85E+001 | −7.05E+002 | 1.77E+003 | 1.90E+003 | −22 | 43093.29 | 45488.54 | |
| TBB_POLAG | −9.12E+002 | −1.12E+003 | 2.29E+003 | 2.71E+003 | −24 | 43428.91 | 45898.77 | |
| TBB_PSEAM | −1.11E+003 | −1.06E+003 | 1.54E+003 | 2.18E+003 | −25 | 43784.36 | 46877.34 | |
| TBBQ_HUMAN | −2.77E+002 | −7.68E+002 | 5.86E+002 | 1.00E+003 | −18 | 42440.37 | 44994.21 | |
| TBB_RHYSE | −1.13E+003 | −1.49E+003 | 3.08E+003 | 3.60E+003 | −21 | 43739.88 | 46423.03 | |
| TBB_SCHCO | −7.83E+002 | −1.13E+003 | 1.60E+003 | 2.11E+003 | −25 | 43970.42 | 46854.63 | |
| TBB_SCHPO | −4.48E+002 | −8.91E+002 | 2.11E+003 | 2.33E+003 | −27 | 43446.86 | 45913.99 | |
| TBB_STRPU | −6.17E+002 | −1.36E+003 | 3.07E+003 | 3.41E+003 | −18 | 29275.73 | 31526.12 | F |
| TBB_STYLE | −9.53E+002 | −1.02E+003 | 2.15E+003 | 2.56E+003 | −24 | 43277.04 | 45763.19 | |
| TBB_TETPY | −5.66E+002 | −8.36E+002 | 1.78E+003 | 2.05E+003 | −24 | 43557.91 | 46339.63 | |
| TBB_TETTH | −5.22E+002 | −8.68E+002 | 1.71E+003 | 1.99E+003 | −25 | 43553.05 | 46384.46 | |
| TBB_THAWE | −9.42E+002 | −1.14E+003 | 2.35E+003 | 2.78E+003 | −23 | 43337.8 | 45963.07 | |
| TBB_TOXGO | −1.44E+003 | −1.21E+003 | 2.28E+003 | 2.96E+003 | −27 | 43887.58 | 46652.72 | |
| TBB_TRYBR | −9.50E+001 | −1.16E+003 | 1.42E+003 | 1.84E+003 | −24 | 43475.69 | 45924.44 | |
| TBB_TRYCR | −5.27E+002 | −1.06E+003 | 1.56E+003 | 1.96E+003 | −25 | 43396.83 | 45908.56 | |
| TBB_VENIN | −1.12E+003 | −1.36E+003 | 3.10E+003 | 3.56E+003 | −22 | 43549.74 | 46256.71 | |
| TBBX_HUMAN | −1.07E+003 | −1.20E+003 | 2.50E+003 | 2.97E+003 | −24 | 43586.17 | 46372.26 | |
| TBB_YEAST | −1.38E+003 | −3.14E+002 | 3.25E+003 | 3.54E+003 | −31 | 44568.68 | 47245.65 | |
| TBD_H U MAN | −2.52E+002 | −1.29E+003 | 3.67E+002 | 1.36E+003 | −5 | 44650.69 | 45579.63 | |
| TBE_HUMAN | 5.31E+002 | −4.99E+002 | 4.47E+002 | 8.55E+002 | −6 | | | |
| TBG1 U MAN | 7.16E+002 | −1.58E+003 | −6.03E+002 | 1.84E+003 | −10 | 44645.36 | 45231.14 | |
| TBG1 _MAIZE | 8.07E+002 | −1.90E+003 | −3.86E+002 | 2.10E+003 | −10 | 46058.94 | 46110.79 | |
| TBG1 _MOUSE | 5.56E+002 | −1.71E+003 | −8.73E+002 | 2.00E+003 | −11 | 44751.95 | 45706.51 | |
| TBG2_ARATH | 1.46E+003 | −2.04E+003 | 5.26E+002 | 2.56E+003 | −10 | 46598.89 | 47773.51 | |
| TBG2_DROM E | 8.15E+002 | −1.58E+003 | −8.50E+002 | 1.97E+003 | −6 | 44800.18 | 45401.53 | |
| TBG2_EU PCR | 3.78E+002 | −1.86E+003 | −7.45E+002 | 2.04E+003 | −15 | 45632.97 | 46882.17 | |
| TBG2_EUPOC | 4.42E+001 | −2.22E+003 | −3.12E+002 | 2.24E+003 | −10 | 45771.97 | 47628.98 | |
| TBG2_HUMAN | 5.46E+002 | −1.57E+003 | −3.46E+002 | 1.70E+003 | −13 | 44707.12 | 45746.7 | |
| TBG2_MAIZE | 4.62E+002 | −1.85E+003 | −5.21E+002 | 1.98E+003 | −12 | | | |
| TBG2_MOUSE | 3.57E+002 | −1.22E+003 | −6.91E+002 | 1.45E+003 | −10 | 44770.54 | 45966.96 | |
| TBG2_ORYSA | 7.37E+002 | −1.71E+003 | −6.59E+002 | 1.97E+003 | −12 | 46151.43 | 46463.29 | |
| TBG3_MAIZE | 7.36E+002 | −1.95E+003 | −1.05E+002 | 2.09E+003 | −9 | 41586.56 | 42200.31 | F |
| TBG_ANEPH | 1.48E+003 | −2.35E+003 | 3.46E+002 | 2.79E+003 | −9 | 46391.54 | 47490.67 | |
| TBG_CAEEL | 3.04E+002 | −1.06E+003 | −8.91E+002 | 1.42E+003 | −9 | 43944.74 | 45972.73 | |
| TBG_CANAL | 1.34E+003 | −1.39E+003 | 1.90E+003 | 2.71E+003 | −23 | | | |
| TBG_CHLRE | 7.24E+002 | −1.85E+003 | −3.37E+002 | 2.02E+003 | −6 | 45684.61 | 46543.6 | |
| TBG_COCHE | 4.43E+002 | −8.17E+002 | −5.81E+002 | 1.10E+003 | −2 | 26054.95 | 27657.99 | F |
| TBG_EMENI | 7.59E+002 | −1.72E+003 | −7.19E+002 | 2.01E+003 | −9 | 44602.99 | 46275.01 | |
| TBG_ENTHI | 1.65E+002 | −9.20E+002 | −8.38E+002 | 1.26E+003 | −6 | 45398.09 | 46350.19 | |
| TBG_EUPAE | 7.82E+002 | −1.99E+003 | −3.07E+002 | 2.16E+003 | −10 | 45766.71 | 47108.63 | |
| TBG_NEUCR | 5.63E+002 | −1.98E+003 | −3.09E+002 | 2.08E+003 | −9 | 45255.26 | 46777.78 | |
| TBG_PHYPA | 1.25E+003 | −2.49E+003 | 2.51E+001 | 2.78E+003 | −8 | 46549.14 | 47781.9 | |
| TBG_PLAFO | 6.66E+002 | −2.18E+003 | −7.53E+002 | 2.40E+003 | −7 | 45179.34 | 46542.13 | |
| TBG_RETFI | 1.16E+003 | −1.59E+003 | −5.16E+002 | 2.04E+003 | −4 | 47100.48 | 48598.68 | |
| TBG_SCHJP | 1.95E+000 | −1.81E+003 | −6.21E+002 | 1.91E+003 | −7 | 44087.45 | 45523.53 | |
| TBG_SCHPO | 3.32E+002 | −1.54E+003 | −3.58E+002 | 1.62E+003 | −8 | 43930.03 | 45423.04 | |
| TBG_USTVI | 7.32E+002 | −1.61E+003 | −8.74E+002 | 1.97E+003 | −10 | 45915.36 | 47039.01 | |
| TBG_XENLA | 8.58E+002 | −1.48E+003 | −8.55E+002 | 1.91E+003 | −9 | 44698.46 | 45367.78 | |
| TBG_YEAST | 9.08E+002 | −1.50E+003 | 1.31E+003 | 2.19E+003 | −30 | 45777.95 | 47349.09 | |

As is also disclosed in the Tuszynski paper, “FIG. 1a shows a scatter diagram of the net/charge/volume ratios of the different tubulins. This plot is striking in that the net charge on the beta-tubulins is bar far the greatest, ranging between −17 and −32 elementary charges (e) depending upon the particular beta-tubulin with an average value in this case at approximately −25e. Next comes the alpha-tubulins whose net charges vary between −10 and 1-25 elementary charges . . . There appears to be little if any correlation between the size of a protein and its charge . . . . Further, it should be kept in mind, that the charge on a tubulin dimmer will be neutralized in solution due to the presence of counter-ions which almost completely screen the net charge. This was experimentally determined for tubulin by the application of an external electric field; the resulting value of an unscreened charge of approximately 0.2e per monomer was found Stracke et al. 2002.” The reference to Stracke et al. was to an article by R. Stracke, J. A. Tuszynski, et al. regarding “Analysis of the migration behaviour of single microtubules in electric fields,” Biochemical and Biophysical Research Communications, 293:606-609, 2002.

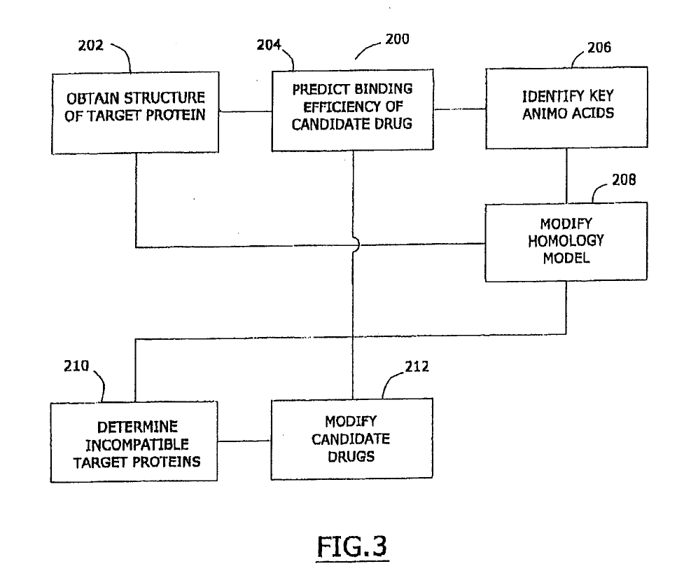

As is also disclosed in the Tuszynski paper, “What is, however, of great interest in connection with polymerization of tubulin into microtubules and with drug-protein binding is the actual distribution of charges on the surface of the tubulin. FIG. 3 illustrates this for the Downing-Nogales structure with plus signs indicating the regions of positively charged and minus signs negatively charged locations. This figure shows C-termini in two very upright positions. Of course, each of the different tubulins will show differences in this regard . . . .”

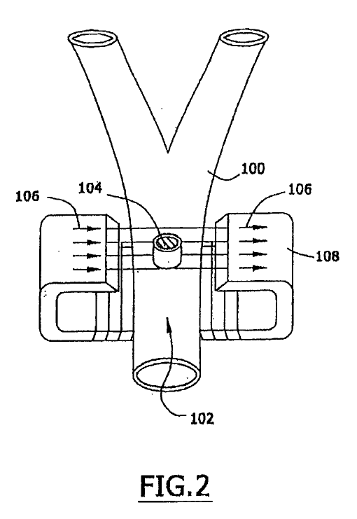

As is also disclosed in the Tuszynski paper, “ . . . alpha tubulins have relatively low dipole moments about their centres-of-mass, ranging between 1000 and 2000 Debye, while the beta-tubulins are very high in this regard with the corresponding values ranging between 1000 and 4000 Debye and with the average value close to 3000 Debye . . . . In FIG. 2 we have illustrated the important aspect of dipole organization for tubulin, namely its orientation. FIG. 2a shows a Mollweide projection of dipole orientation in tubulin . . . . We conclude from this diagram and its magnification . . . that both alpha- and beta-tubulins orient their dipose moments in a direction that is close to being perpendicular to the microtubule surface . . . .”

As is also disclosed in the Tuszynski paper, “FIG. 1c shows the logarithm of surface area against the logarithm of volume for the different tubulins . . . . Note that the alpha and beta families have a very similar slope with a value close to the unity that is indicative of cylindrical symmetry in the overall geometry . . . .”

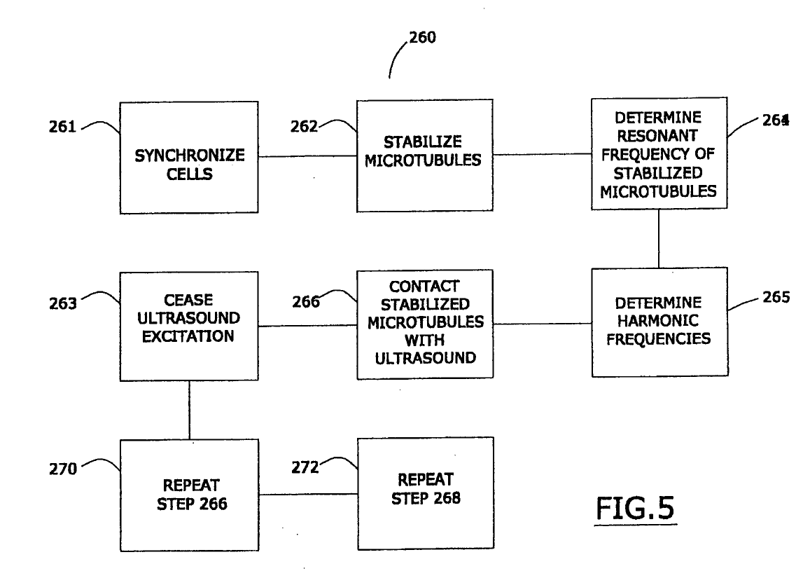

As is also disclosed in the Tuszynski paper, “Our models show that only alpha- and beta-tubulins have C-termini that project outwards from the tubulin, due to their high negative charges. FIG. 5 shows the energy levels of different orientational positons of the C-termini in a toy model and suggests that there is relatively little energetic difference between projecting straight outward from the rest of the tublin and lying on the surface of tubulin in certain energy minima . . . .”

As is also disclosed in the Tuszynski et al. paper, “Isotype compositon has a demonstrable effect on microtubule assembly kinetics (Panda et al., 1994).” The Panda et al. reference was an article by D. Panda et al. on “Microtubule dynamics in vitro are regulated by the tubulin isotype composition,” Proc. Natl. Acad. Sci. USA 91: 11 358-11 362, 1994.

As is also disclosed in the Tuszynski paper, “This could be due to changes in the electrostatics of tubulin, which although significantly screened by counter-ions does affect microtubule assembly by influencing dimer-dimer interactions over relatively short distances (approximagely 5 nm) as well as the kinetics of assembly. These short-range interactions have recently been studied by Sept et al. (2003) by calculating the energy of protofilament-protofilaent interactions. These authors condluced from their work that the two types of microtubule lattices (A and B) correspond to the local energy minima.” The Sept et al. reference was to an article by D. Sept et al., “The physical basis of microtubule structure and stability,” Protein Science, 12:2257-2261, 2003.

As is also disclosed in the Tuszynski paper, “The dipole moment could play a role in microtubule assembly and in other processes. This could be instrumental in the docking process of molecules to tubulin and in the proper steric configuration of a tubulin dimer as it approaches a microtubule for binding. An isolated dimer has an electric field dominated by its net charge . . . . In contrast, a tubulin dimer . . . surrounded by water molecules and counter-ions, as is physiologically relevant, has an isopotential surface with two lobes much like the dumbbell shape of a mathematically dipole moment. The greater the dipole of of each of its units is, the less stable the microtubule since dipole-dipole interactions provide a positive energy disfavoring a microtubule structure. Note that the strength of the interaction potential is proportional to the square of the dipole moment, hence microtubule structures formed from tubulin units with larger dipoles momements should be more prone to undergo disassembly catastrophes compoared to those microtubles that contain low dipole moment tubulins. For organisms that express more than one type of tubulin isotype in the same cell, one can conceive that microtubule dynamic behavior could be regulated by altering the relative amounts of the different isotypes according to their dipole moments.”

As is also disclosed in the Tuszynski paper, “In terms of surface/volume ratios, α- and β-tubulin are the least compact, while γ, δ and ε are the most compact. There is abundant evidence that both α and β have flexible conformations. This is attested to by their interaction with drugs and is consistent with the dynamic instability of microtubules. In contrast, there is as yet no evidence of dynamic instability in γ, δ and ε partcipating in dynamic instability, nor is there any theoretical reason to imagine such flexibility. It is reasonable to postulate that a less compact structure may have a more flexible conformation.”

As is also disclosed in the Tuszynski et al. paper, “Our models predict that the C-termini of α and β can readily adopt the two extreme conformations: either projecting outwards from the tubulin (and the microtubule surface) or to lie on the surface, albeit such that their charged residues can form electrostatic bonds with complimentary charges on the surface. The state of the C-terminus (upright, down, or in intermediate states) down) is easily influenced by the local ion concentration including pH. This conformational complexity has many implications (Pal et al., 2001).” The Pal et al. reference is an article by D. Pal et al. on “Conformational properties of alpha-tubulin tail peptide: implications for tail-body interaction,” Biochemistry, 40: 15 512-15 519, 2001.

As is also disclosed in the Tuszynski paper, “First, a projecting C-terminus could play a major role in signaling. The fact that tubulin isotypes differ markedly in the C-termini suggests that specific sequences may mediate the functional roles of the isotypes. These sequences would be readily available for interactions with other proteins in a projecting C-terminus. Second, the C-termini are the sites of many of the post-translational modifications of tubulin—-polyglutamylation, polyglycylation, detyrosinolation/tyrosinolation, removal of the penultimate glutamic acid, and phosphorylation of serine and tyrosine (Redeker et al., 1998).” The Redeker et al. reference was an article by V. Redekere et al. on “Posttranslational modifications of the C-terminus of alpha-tubulin in adult rat brain: alpha 4 is glutamylated at two residues,” Biochemistry, 37: 14 838-14 844, 1998.

As is also disclosed in the Tuszynski paper, “It is known that the C-termini are essential to normal microtubule function (Duan and Gorovsky, 2002); a projecting C-terminus would be easily accessible to enzymes that affect these modifications and also the modification could influence the likelihood of the C-terminus changing conformation. In addition, if the modification plays a role in signaling then the signal would be readily available in a projecting C-terminus, as already mentioned.” The reference to Duan and Gorovsky is to an article by J. Duan et al., “Both carboxy-termianl tails of alpha- and beta-tubulin are essential, but either one will suffice,” Current Biology, 12:313-316, 2002.

As is also disclosed in the Tuszynski et al. paper, “Third, projecting C-termini would automatically create spacing between microtubules. It is known that microtubules are never closely packed and are surrounded by what is referred to as an exclusion zone. (Dustin, 1984).” The reference to Dustin is to a book by P. Dustin on “Microtubules (Springer-Verlag, Berlin, 1984).

As is also disclosed in the Tuszynski paper, “This is a region of space around them that strongly disfavors the presence of other microtubules in the vicinity. Although MAPs could play a role in such spacing, electrostatic repulsion among C-terminal ends are likely to influence this as well. The C-termini are the major sites of binding of the MAPs to tubulin. A projecting C-terminus may facilitate MAP binding and, conversely, MAP binding could influence the conformation of the C-terminus. Evidence for this is provided by the work of Makridis et al who showed that when tau binds to microtubules, it triggers a structural change on the microtubule surface whereby a structural element, presumably tau, lies along the surface of the microtubule forming a lattice whose alingement angle is much sharper than that of the tubulin subunits. This lattice is presumably superimposed on top of the normal microtubule (A or B) lattice. The orientation of the C-termini when they are lying on the surface of the microtubule form exactly the same kind of lattice that (Makridis et al, 2003) observed, a striking confirmation of the potential accuracy of our modeling . . . . These results raise the possibility that the orientation of the C-termini of the alpha and beta subunits determines the arrangement of tau molecules on the microtubule.” The Makrides reference referred to is an article by V. Markrides et al., “Microtubule-dependent oligomerization of tau: Implicatons for physiological tau function and tauopathies,” J. Biol. Chem., 278:33 298-33 304, 2003.

As is also disclosed in the Tuszynski et al. paper, “ . . . the state of the C-termini could mediate how motor proteins such as kinesin bind to and move on microtubules. Our models show that kinesin can only bind to upright C-termini . . . and not to C-termini lying on the surface of the microtubule . . . . Very minor changes in the local ionic environment or the pH could halt the progress of kinesin by collapsing the C-termini. One can postulate that the proportion of C-termini that are in the upright conformation in a given portion of the microtubule could determine the actual rate of kinesin movement. It is likely that such arguments could apply to other motor proteins as well. One might imagine that the very fine coordination of movements that occur in processes such as mitosis could be influenced or even caused by the conformational state of the C-termini in particular areas of the microtubule.”

As is also disclosed in the Tuszynski paper, “Finally, one can imagine that the C-termini could collapse in waves that could simultaneously move a wave of ions that could polarize or depolarize a membrane. This could be a form of microtubule signaling that has not yet been considered. A quantitative model of ionic wave transmission coupled to coordinated motion of the C-termini of dendritic microtubules has been recently developed by Priel et al . . . .” The refererence to Priel et al. was to an article by A. Priel et al. entitled “Moleuclar Dynamics of C-termini in Tubulin: Implications for Transport to Active Synapsis,” submitted to Biophys. J., 2003.

Table 1 of the Tuszynksi paper disclosed the tubulin sequences used in the study reported in the article. In such Table 1, the table names the names the source organism, and for each α, β, γ, δ, and ε, gives the name used in the databank.

The Use of Particular Models of Isotypes of Tubulin for Drug Development

In one embodiment of the invention, once a particular tubulin isotype has been identified as being of interest, and once a three-dimensional model of it has been made in accordance with the process of this invention, this model may then be used to identify which drug or drugs would most advantageously interact with the binding sites of the tubulin isotype in question.

The preferred binding sites which may be used in the process of identifying the candidate drugs are discussed in the next section of this specification.

Preferred Binding Sites of Tubulin Isotypes

It is known that many chemotherapeutic drugs effect their primary actions by inhibiting tubulin polymerization. Thus, as is disclosed in U.S. Pat. No. 6,162,930 (the entire disclosure of which is hereby incorporated by reference into this specification), “An aggressive chemotherapeutic strategy toward the treatment and maintenance of solid-tumor cancers continues to rely on the development of architecturally new and biologically more potent anti-tumor, anti-mitotic agents. A variety of clinically-promising compounds which demonstrate potent cytotoxic and anti-tumor activity are known to effect their primary mode of action through an efficient inhibition of tubulin polymerization (Gerwick et al.). This class of compounds undergoes an initial binding interaction to the ubiquitous protein tubulin which in turn arrests the ability of tubulin to polymerize into microtubules which are essential components for cell maintenance and cell division (Owellen et al.).”

U.S. Pat. No. 6,162,930 also discloses that the precise means by which the cytotoxic agents “ . . . arrests the ability of tubulin to polymerize . . . ” is unknown, stating that: “Currently the most recognized and clinically useful tubulin polymerization inhibitors for the treatment of cancer are vinblastine and vincristine (Lavielle, et al.). Additionally, the natural products rhizoxin (Nakada, et al., 1993a and 1993b; Boger et al.; Rao et al., 1992 and 1993; Kobayashi et al., 1992 and 1993) combretastin A-4 and A-2 (Lin et al.; Pettit, et al., 1982, 1985, and 1987) and taxol (Kingston et al.; Schiff et al; Swindell, et a, 1991; Parness, et al.) as well as certain synthetic analogues including the 2-styrylquinazolin-4(3H)-ones (SQO) (Jiang et al.) and highly oxygenated derivatives of cis- and trans-stilbene (Cushman et al.) and dihydrostilbene are all known to mediate their cytotoxic activity through a binding interaction with tubulin. The exact nature of this interaction remains unknown and most likely varies somewhat between the series of compounds.”

U.S. Pat. No. 6,512,003 also discusses the “ . . . nature of this unknown interaction . . . ,” stating that (at column 1) “Novel tubulin-binding molecules, which, upon binding to tubulin, interfere with tubulin polymerization, can provide novel agents for the inhibition of cellular proliferation and treatement of cancer.” U.S. Pat. No. 6,512,003 presents a general discussion of the role of tubulin in cellular proliferation, disclosing (also at colum1) that: Cellular proliferation, for example, in cancer and other cell proliferative disorders, occurs as a result of cell division, or mitosis. Microtubules play a pivotal role in mitotic spindle assembly and cell division . . . . These cytoskeletal elements are formed by the self-association of the ad tubulin heterodimers . . . . Agents which induce depolymerization of tubulin and/or inhibit the polymerization of tubulin provide a therapeutic approach to the treatment of cell proliferation disorders such as cancer. Recently, the structure of the .alpha.β tubulin dimer was resolved by electron crystallography of zinc-induced tubulin sheets . . . . According to the reported atomic model, each 46×40×65 .ANG. tubulin monomer is made up of a 205 amino acid N-terminal GTP/GDP binding domain with a Rossman fold topology typical for nucleotide-binding proteins, a 180 amino acid intermediate domain comprised of a mixed β sheet and five helices which contain the taxol binding site, and a predominantly helical C-terminal domain implicated in binding of microtubule-associated protein (MAP) and motor proteins . . . .”

U.S. Pat. No. 6,512,003 also teaches that the the binding site of vinca alkaloids to tubulin differs from the binding site of colchicin to tublin, stating (also at column 1) that: “Spongistatin (SP) . . . is a potent tubulin depolymerizing natural product isolated from an Eastern Indian Ocean sponge in the genus Spongia . . . . Spongistatins are 32-membered macrocyclic lactone compounds with a spongipyran ring system containing 4 pyran-type rings incorporated into two spiro[5.5]ketal moieties . . . In cytotoxicity assays, spongistatin (SP) exhibited potent cytotoxicity with subnanomolar IC50 values against an NCI panel of 60 human cancer cell lines . . . . SP was found to inhibit the binding of vinc alkaloids (but not colchicin) to tubulin . . . , indicating that the binding site for this potent tubulin depolymerizing agent may also serve as a binding region for vinc alkaloids.”

U.S. Pat. No. 6,593,374, the entire disclsoure of which is hereby incorporated by reference into this specification, presents a “working hypothesis” that the “ . . . methoxy aryl functionality . . . ” is especially important for binding at the colchicin binding site. It discloses (at columns 1-2 thereof) that: “An important aspect of this work requires a detailed understanding, on the molecular level, of the ‘small molecule’ binding domain of both the alpha. and β subunits of tubulin. The tertiary structure of the alpha.,β tubulin heterodimer was reported in 1998 by Downing and co-workers at a resolution of 3.7 ANG. using a technique known as electron crystallography . . . . This brilliant accomplishment culminates decades of work directed toward the elucidation of this structure and should facilitate the identification of small molecule binding sites, such as the colchicine site, through techniques such as photoaffinity and chemical affinity labeling . . . . We have developed a working hypothesis suggesting that the discovery of new antimitotic agents may result from the judicious combination of a molecular template (scaffold) which in appropriately substituted form (ie. phenolic moieties, etc.) interacts with estrogen receptor (ER), suitably modified with structural features deemed imperative for tubulin binding (arylalkoxy groups, certain halogen substitutions, etc.). The methoxy aryl functionality seems especially important for increased interaction at the colchicine binding site in certain analogs . . . . Upon formulation of this hypothesis concerning ER molecular templates, our initial design and synthesis efforts centered on benzo[b]thiophene ligands modeled after raloxifene, the selective estrogen receptor modulator (SERM) developed by Eli Lilly and Co . . . . Our initial studies resulted in the preparation of a very active benzo[b]thiophene-based antitubulin agent . . . . In further support of our hypothesis, recent studies have shown that certain estrogen receptor (ER) binding compounds as structurally modified estradiol congeners (2-methoxyestradiol, for example) interact with tubulin and inhibit tubulin polymerization . . . . Estradiol is, of course, perhaps the most important estrogen in humans, and it is intriguing and instructive that the addition of the methoxy aryl motif to this compound makes it interactive with tubulin. It is also noteworthy that 2-methoxyestradiol is a natural mammalian metabolite of estradiol and may play a cell growth regulatory role especially prominent during pregnancy. The term ‘phenolic moiety’ means herein a hydroxy group when it refers to an R group on an aryl ring.”

As is also disclsoed in U.S. Pat. No. 6,593,374 (at column 1 thereof), “Tubulin is currently among the most attractive therapeutic targets in new drug design for the treatment of solid tumors. The heralded success of vincristine and taxol along with the promise of combretastatin A-4 (CSA-4) prodrug and dolastatin . . . , to name just a few, have firmly established the clinical efficacy of these antimitotic agents for cancer treatment. An aggressive chemotherapeutic strategy toward the treatment and maintenance of solid-tumor cancers continues to rely on the development of architecturally new and biologically more potent anti-tumor, anti-mitotic agents which mediate their effect through a direct binding interaction with tubulin. A variety of clinically-promising compounds which demonstrate potent cytotoxicity and antitumor activity are known to effect their primary mode of action through an efficient inhibition of tubulin polymerization . . . . This class of compounds undergoes an initial interaction (binding) to the ubiquitous protein tubulin which in turn arrests the ability of tubulin to polymerize into microtubules which are essential components for cell maintenance and division . . . . During metaphase of the cell cycle, the nuclear membrane has broken down and the cytoskeletal protein tubulin is able to form centrosomes (also called microtubule organizing centers) and through polymerization and depolymerization of tubulin the dividing chromosomes are separated. Currently, the most recognized and clinically useful members of this class of antimitotic, antitumor agents are vinblastine and vincristine . . . along with taxol . . . . Additionally, the natural products rhizoxin, . . . combretastatin A-4 and A-2, . . . curacin A, . . . podophyllotoxin, . . . epothilones A and B, . . . dolastatin 10 . . . and welwistatin . . . (to name just a few) as well as certain synthetic analogues including phenstatin, . . . the 2-styrylquinazolin-4(3H)-ones (SQO), . . . and highly oxygenated derivatives of cis- and trans-stilbene . . . and dihydrostilbene are all known to mediate their cytotoxic activity through a binding interaction with tubulin. The exact nature of this binding site interaction remains largely unknown, and definitely varies between the series of compounds.”

Published U.S. patent application 2004/0044059, the entire disclosure of which is hereby incorporated by reference into this specification, also discloses the uncertaintly that exists with regard to the “ . . . tubulin binding site interactions . . . ” At page 1 thereof, it states that: “The exact nature of tubulin binding site interactions remain largely unknown, and they definitely vary between each class of Tubulin Binding Agent. Photoaffinity labeling and other binding site elucidation techniques have identified three key binding sites on tubulin: 1) the Colchicine site (Floyd et al, Biochemistry, 1989; Staretz et al, J. Org. Chem., 1993; Williams et al, J. Biol. Chem., 1985; Wolff et al, Proc. Natl. Acad. Sci. U.S.A., 1991), 2) the Vinca Alkaloid site (Safa et al, Biochemistry, 1987), and 3) a site on the polymerized microtubule to which taxol binds (Rao et al, J. Natl. Cancer Inst., 1992; Lin et al, Biochemistry, 1989; Sawada et al, Bioconjugate Chem, 1993; Sawada et al, Biochem. Biophys. Res. Commun., 1991; Sawada et al, Biochem. Pharmacol., 1993). An important aspect of this work requires a detailed understanding, at the molecular level, of the ‘small molecule’ binding domain of both the α and β subunits of tubulin. The tertiary structure of the α,β tubulin heterodimer was reported in 1998 by Downing and co-workers at a resolution of 3.7 using a technique known as electron crystallography (Nogales et al, Nature, 1998). This brilliant accomplishment culminates decades of work directed toward the elucidation of this structure and should facilitate the identification of small molecule binding sites, such as the colchicine site, using techniques such as photoaffinity and chemical affinity labeling (Chavan et al, Bioconjugate Chem., 1993; Hahn et al, Photochem. Photobiol., 1992).”

As is also disclosed in published U.S. patent application 2004/0044059, “The cytoskeletal protein tubulin is among the most attractive therapeutic drug targets for the treatment of solid tumors. A particularly successful class of chemotherapeutics mediates its anti-tumor effect through a direct binding interaction with tubulin. This clinically-promising class of therapeutics, called Tubulin Binding Agents, exhibit potent tumor cell cytotoxicity by efficiently inhibiting the polymerization of αβ-tubulin heterodimers into the microtubule structures that are required for facilitation of mitosis or cell division (Hamel, Medicinal Research Reviews, 1996). . . . Currently, the most recognized and clinically useful antitumor agents are Vinca Alkaloids, such as Vinblastine and Vincristine (Owellen et al, Cancer Res., 1976; Lavielle et al, J. Med. Chem., 1991) along with Taxanes such Taxol (Kingston, J. Nat. Prod., 1990; Schiff et al, Nature, 1979; Swindell et al, J. Cell Biol., 1981). Additionally, natural products such as Rhizoxin (Nakada et al, Tetrahedron Lett., 1993; Boger et al, J. Org. Chem., 1992; Rao, et al, Tetrahedron Lett., 1992; Kobayashi et al, Pure Appl. Chem., 1992; Kobayashi et al, Indian J. Chem., 1993; Rao et al, Tetrahedron Lett., 1993), the Combretastatins (Lin et al, Biochemistry, 1989; Pettit et al, J. Nat. Prod., 1987; Pettit et al, J. Org. Chem., 1985; Pettit et al, Can. J. Chem., 1982; Dorr et al, Invest. New Drugs, 1996), Curacin A (Gerwick et al, J. Org. Chem., 59:1243, 1994), Podophyllotoxin (Hammonds et al, J. Med. Microbiol, 1996; Coretese et al, J. Biol. Chem., 1977), Epothilones A and B (Nicolau et al., Nature, 1997), Dolastatin-10 (Pettit et al, J. Am. Chem. Soc., 1987; Pettit et al, Anti-Cancer Drug Des., 1998), and Welwistatin (Zhang et al, Molecular Pharmacology, 1996), as well as certain synthetic analogues including Phenstatin (Pettit G R et al., J. Med. Chem., 1998), 2-styrylquinazolin-4(3H)-ones (“SQOs”, Jiang et al, J. Med. Chem., 1990), and highly oxygenated derivatives of cis- and trans-stilbene and dihydrostilbene (Cushman et al, J. Med. Chem., 1991) are all known to mediate their tumor cytotoxic activity through tubulin binding and subsequent inhibition of mitosis.”

As is also disclosed in published U.S. patent application 2004/0044059, “Normally, during the metaphase of cell mitosis, the nuclear membrane has broken down and tubulin is able to form centrosomes (also called microtubule organizing centers) which facilitate the formation of a microtubule spindle apparatus to which the dividing chromosomes become attached. Subsequent polymerization and depolymerization of the spindle apparatus mitigates the separation of the daughter chromosomes during anaphase such that each daughter cell contains a full complement of chromosomes. As antiproliferatives or antimitotic agents, Tubulin Binding Agents exploit the relatively rapid mitosis that occurs in proliferating tumor cells. By binding to tubulin and inhibiting the formation of the spindle apparatus in a tumor cell, the Tubulin Binding Agent can cause significant tumor cell cytotoxicity with relatively minor effects on the slowly-dividing normal cells of the patient.”

An article by Mary Ann Jordan et al., entitled “Microtubules as a target for anticancer drugs,” appeared in Nature Reviews/Cancer, Volume 4, April 2004, pages 253-266. At page 253 of this article, it was disclosed that: “Microtubles are extremely important in the process of mitosis . . . . Their importance in mitosis and cell divison makes microtubles an important target for anticancer drugs. Microtubules and their dynamics are the targets of a chemically diverse group of antimitotic drugs (with various tubulin-binding sites) that have been used with great success in the treatment of cancer . . . . In view of the success of this class of drugs, it has been argued that microtubules represent the best cancer target to be identified so far . . . .”

The polymerization dynamics of microtubules are discussed at pages 254 et seq. of the Jordan paper, wherein it is disclosed that: “The polymerization if microtubules occurs by a nucleation-elongation mechanism in which the relatively slow formation of a short microtubule ‘nucleus’ is followed by rapid elongation of the microtubule at its ends by the reversible, non-covalent addition of tubulin dimers . . . . It is important to emphasize that microtubues are not simple equilibrium polymers. The show complex polymerization dynamics that use energy provided by the hydrolysis of GTP at the time that tubulin with bound GTP adds to the microtubule ends; these dynamics are crucial to their cellular functions.”

The Jordan et al. article also disloses that: “ . . . the correct movements of the chromosomes and their proper segregation to daughter cells require extremely rapid dynamics, making mitosis exquisitely sensitive to microtubule-targeted drugs.”

The Jordan et al. article also disloses that: “The biological functions of microtubules in all cells are determined and regulated in large part by their polymerization dynamics . . . . Microtubules show two kinds of non-equilibrium dynamics, both with purified microtubule systes in vitro and in cells.”

The Jordan et al. article also discloses (at page 257, “Box 1”) how one may measure microtubule dynamic instability. It states that: “With purified microtubules in vitro (generally purified from pig, cow, or sheep brains, which are a rich source of microtubules), dynamic instability of individual microtubules is measured by computer-enhanced time-lapse differential interference contrast microscopy. In living cells, individual fluorescent microtubules can be readily visualized in the thin peripheral regions of the cells after microinjection of fluorescent tubulin or by expressnion of GFP (green fluorescent protein) labeled tubulin. The growing and shortening dynamics of the microtubules, which are prominent in this region of interphase cells, are recorded by time-lapse using a sensitive CCD (charge-coupled device) camera. To determine how microtubule length changes with time, both in vitro and in living cells, the ends of the individual growing and shortening microtubules are traced by a cursor on succeeding time-lapse frames, recorded, and their rates, lengths, and durations of growing and shortening are calculated from the sequence of record x−=y positons of the microtubule ends.”

The “dynamic instability” phenomenon is discussed at page 254 of the Jordan et al. article, wherein it is disclosed that: “One kind of dynamic behavior that is highly prominent in cells, called ‘dynamic instability,’ is a process in which the individual microtubule ends switch between phases of growth and shortening . . . The two ends of a microtubule are not equivalent: one end, called the plus end, grows and shortens more rapidly and more extensively than the other (the minus end). . . . The microtubules undergo relatively long periods of slow lengthening, brief periods of rapid shortening, and periods of attenuated dynamics or pause, when the microtubules neither gorw nor shorten detectably . . . . Dynamic instability is characterized by four main variables: the rate of microtubule growth; the rate of shortening; the frequency of transition from the growth or paused state to shortening (this transitionis called a ‘catastrophe’); and the frequency of transition from shortening to growth or pause (called a ‘rescue’). Periods of pause are defined operationally, when any changes in microtubule length that might be occurring are below the resolution of the light microscope. The variable called ‘dynamicity’ is highly useful to describve the overall visually detectable rate of exchange of tubulin dimmers with microtubule ends.

The Jordan et al. article also discloses that: “The second dynamic behavior, called ‘treadmilling’ . . . is net growth at one microtubule end and balanced net shortening at the opposite end . . . It involves the intrinsic flow of tubulin subunits from the plus end of the microtubule to the minus end and is created by differences in the critical subunit concentrations at the opposite microtubule ends. (The critical subunit concentrations are the concentrations of the free tubulin subunits in equilibrium with the microtubule ends.). This behavior occurs in cells as well as in vitro and might be particularly important in mitosis . . . . Treadmilling and dynamic instability are compatible behaviours, and a specific microtubule population can show primary treadmilling behavior, dynamic instability behaviour, or some mixture of both. The mechanisms that control one or the other behavior are poorly understood but probably involve the tubulin isotype compositon of the microtubule poplulation, the degree of post-transaltional modification of tubulin, and, especially, the actions of regulatory proteins.” Applicants believe that, by causing the combination of one or more particular tubulin isotypes with a candidate therapeutic agent, one may affect the treadmiling behaviour and/or the dynamic instability behaviour of the microtubules which comrprise the tubulin isotype.” In particular, they believe that the magnetic anti-mitotic compound of their invention affects the treadmilling behavior and/or the dynamic instability behavior of microtubules.

As is disclosed on page 263 of the Jordan et al. article, a comprehensive review of tubulin isotypes and post-translational modifications is presented in an article by R. F. Luduena, “Multiple forms of tubulin: different gene productrs and covalent modifications,” Int. Rev. Cytology, 170: 207-275 (1998). The Jordan et al. article also refers to a work by P. Verdier-Pinard et al., “Direct analysis of tubulin expression in cancer cell lines by electrospray ionization mass spectrometery,” Biochemistry, 42: 12019-12027 (2003). According to the Jordan et al. article, “The Verider-Pinard et al. article describes analyses of tubulin isotypes, muations, and post-translational modifications by liquid chromatography/electrospray-ionization mass spectrometery in paclitaxel-sensivite and resistant cell lines.”

Referring again to the Jordan et al. article, it is disclosed that: “Dynamic instability and treadmilling behaviours can both be observed with purified microtubules in vitro. However, the rate and extent of both treadmilling and dynamc instability are relatively slow with purified microtubules compared with rates in cells. It is clear that microtubule dynamics in cells are regulated by a host of mechanisms: cells can alter their expression levels of β tubulin isotypes; they can alter their levels of tubulin post-translational modifications; they can express mutated tubulin; and they can alter the expression and phosphorylation levels of microtubule-regulatory proteins . . . that interact with the microtubule surfaceds and ends. Although microtubule dynamics can be modulated by the interaction of regulatory molecules with soluble tubulin itself, the assembled microtubule is likely to the the primary target of cellular molecules that regulate microtubule dynamics. The many drugs that modulate microtubule dynamics might be mimicking the actions of the numerous natural regulators that control microtubule dynamics in cells.” Applicants believe that the magnetic anti-mitotic compound of their invention is as effective as is paclitaxel in “ . . . mimicking the actions of the numerous natural regulators that control microtubule dynamics in cells . . . .”

At page 255 of the Jordan et al. article, the authors disclose that “Microtubule dynamics are crucial to mitosis . . . . With the development of sophisticated methods for observing microtubule dynamics in living cells, it is now possible to visualize the dynamics of mitotic spindle microtubules. With these advances it has become clear that microtubles in mitotic spindles have uniquely rapid dynamics that are crucial to successful mitosis . . . During interphase, microtubules turn over (eschange their tubulin with the soluble tubulin pool) relatively slowly, with half-times that range from several minutes to several hours . . . . The interphase microtubule network disassembles at the onset of mitosis and is replaced by a new population of spindle microtubules that are 4-100 times more dynamic than the microtubules in the interphase cytoskeleton. Although there is variation among the various spindle-microtubule subpopulations, mitotic-spindle microtubules exchange their tubulin with tubulin in the soluble pool rapidly with half-times on the order of 10-30 seconds . . . . At least in some cells, the increase in dynamics seems to result from an increase in the catastrophe frequency, and a reduction in the rescue frequency rather than from changes in the inherent rate of growth and shortening.”

At page 256 of the Jordan et al. article, a “Table 1” is presented regarding “Antimitotic drugs, their diverse binding sites on tubulin and their stages of clinical development.” As is disclosed in such Table 1, one of the well-known binding domains on tubulin is the “vinca domain.”

One drug that binds at the vinca domain is Vinblastine (Velban), which is used to treat Hodgkins disease and testicular germ cell cancer. Reference may be had, e.g., to articles by G. C. Na et al. (“Thermodynamic linkage between tubulin self-association and the binding of vinblastine,” Biochemistry, 19: 1347-1354, 1980; and “Stoichiometry of the vinblastine self-induced self-association of calf-brain tubulin,” Biochem. Soc. Trans., 8: 1347-1354, 1980), by S. Lobert et al. (in Methods in Enzymology, Vol. 323, [ed. Johnson M.] 77-103 [Academic Press 2000]), and by A. Duflos et al. (“Novel aspects of natural and modified vinca alkaloids,” Curr. Med. Chem. Anti-Canc. Agents, 2: 55-70, 2002).

Another drug that binds at the vinca domain is Vincristine (Oncovin); it is used to treat leukemia and lymphomas. Reference may be had, e.g., to works by G. L. Plosker et al. (“Rituximab: a review of its use in non-Hodgkins lymphoma and chronic leukemia,” Drugs, 63: 803-843, 2003), by A. B. Sandler (“Chemotherapy for small cell lung cancer,” Semin. Oncol., 30: 9-25, 2003), and by J. O. Armitage et al. (“Overview of rational and individualized therapeutic strategies for non-Hodgkin's lymphoma,” Clin. Lymphoma, 3: S5-S11, 2002).

Another drug that binds at the vinca domain is Vinorelbine (Navelbine), which is used to treat sold tumors, lymphomas and lung cancer. Reference may be had, e.g., to works by J. Jassem et al. (“Oral vinorelbine in combination with cisplatin, a novel active regimen in advanced non-small-cell lung cancer,” Ann. Oncol. 14: 1634-1639, 2003), by A. Rossi et al. (“Single agent vinorelbine as first-line chemotherapy in elderly patients with advanced breast cancer,” Anticancer Res., 23: 1657-1664, 2003), and by A. D. Seidman (“Monotherapy options in the management of metastatic breast cancer,” Semin. Oncol., 30: 6-10, 2003).

Another drug that binds at the vinca domain is Vinflnine, which is used to treat bladder cancer, non-small-cell lung cancer, and breast cancer. Reference may be had to, e.g., the aforementioned article by A. Duflos et al., and to an article by T. Okouneva et al. on “The effects of vinflunine, vinorelbine, and vinblastine on centromere dynamics,” Cancer Ther., 2: 4.27-4.36, 2003.

Another drug that binds to the vinca domain is cryptophycin 52, and it is used to treat solid tumors. Reference may be had, e.g., to articles by D. Panda et al. (“Interaction of the antitumor compound cryptophycin 52 with tubulin,” Biochemistry, 39: 14121-14127, 2000), and by K. Kerksiek et al. (“Interaction of cryptophycin with tubulin and microtubules,” FEBS Lett., 377: 59-61, 1995).

A class of drugs that binds to the vinca domain of tubulin is the halichondrins (such as, e.g., E7389). Reference may be had, e.g., to articles by M. A. Jordan (“Mechanism of action of antitumor drugs that interact with microtubules and tubulin,” Curr. Med. Chem Anti-Cancer. Agents, 2: 1-17, 2002), by R. B. Bai et al. (“Halichondrin B and homohalichondrin B, marine natural products binding in the Vinca domain of tubulin. Discovery of tubulin-based mechanism of action by analysis of differential cytotoxity data,” J. Biol. Chem., 266: 15882-15889, 1991), by R. F. Luduena et al. (“Interaction of halichondrin B and homohalichondrin B with bovine brain tubulin,” Biochem. Pharmcol., 45: 4.21-4.27, 1993), and by M. J. Towle et al. (in in vitro and in vivo anticancer activities of synthetic macrocyclic ketone analogs of halichondrin B, Cancer Res., 61: 1013-1021, 2001),

Another class of drugs that bind to the vinca domain are the dolastatins (such as TZT-1027), which are used as a vascular targeting agent. Reference may be had, e.g., to an article by E. Harnel, “Natural products which interact with tubulin in the Vinca domain: maytarsine, rhizoxin, phomopsin A. Dolostatins 10 and 15 and halichondrin B.,” Pharmacol. Ther., 55:31-51, 1992.

Another class of drugs that bind to the vinca domain is the hemiasterlins (such as HTI-286). Reference may be had, e.g., to articles by R. Bai et al. (“Interactions of the sponge-derived antimitotic antipeptide hemiasterin with tubulin: comparison with dolastatin 10 and cryptophycin 1,” Biochemistry, 38: 14302-14310, 1999), and by F. Loganzo et al. (“HTI-286, a synthetic analogue of the tripeptide hemiasterin, is a potent antimicrotubule agent that circumvents P-glycoprotein-mediated resistance in vitro and in vivo,” Cancer Res., 63: 1838-1845, 2003).

Another of the binding sites mentioned in the 2004 Jordan et al. article (see Table 1) is the colchicine domain. One of the drugs that binds in the colchicine domain is colchicine, and it is used to treat non-neoplastic diseases such as gout and familial Mediterranean fever. Reference may be had, e.g., to articles by S. B. Hastie (“Interactions of colchicines with tubulin,” Pharmacol. Ther., 512: 377-401, 1991), and by D. Skoufias et al., “Mechanism of inhibition of microtubule polymerization by colchicines inhibitory potencies of unliganded cochicine and tubulin-colchicine complexes,” Biochemistry, 31: 738-746, 1992.

The combretastatins (AVE8062A, CA-1-P, CA-4-P, N-acetylcolchicinol-O-phosphate, ZD6126) are another class of drugs that bind at the colchicines binding site. Reference may be had to articles by G. M. Tozer et al. (“The biology of the combretastatins as tumor vascular targeting agent,” Int. J. Exp. Pathol., 83: 21-38, 2002), and by E. Harnel et al. (“Antitumor 2,3-dihydro-2-(aryl)-4(1H) quinazolinone derivatives: interactions with tubulin,” Biochem. Pharmacol., 51: 53-59, 1996).

Another class of drugs that bind to the colchicines domain is the methoxybenzene-sulphonamides (such as ABT-751, E7010, etc.) that are used to treat solid tumors. Reference may be had, e.g., to an article by K. Yoshimatsu et al., “Mechanism of action of E7010, an orally active sulfonamide antitumor agent: inhibition of mitosis by binding to the colchicines site of tubulin,” Cancer Res., 57: 3208-3213, 1997).

As is also disclosed in Table 1 of the 2004 M. A. Jordan et al. article, the taxane site is another well known tubulin binding site. Taxanes (such as paclitaxel) bind at this site and are used to treat ovarian cancer, breast cancer, lung cancer, Kaposi's sarcoma, and many other tumors. Reference may be had, e.g., to articles by S. B. Horwitz (“How to make taxol from scratch,” Nature, 367: 593-594, 1994), by J. Manfredi et al. (“Taxol binds to cell microtubules,” J. Cell. Biol., 94: 688-696, 1982), by J. Parness et al. (“Taxol binds to polymerized tublulin in vitro,” J. Cell. Biol., 91: 479-487, 1981), and by J. F. Diaz et al. (“Assembly of purified GDP-tubulin into microtubules induced by taxol and taxotere: reversibility, ligand stoichiochemistry, and competition,” Biochemistry, 32: 2747-2755, 1993.).

Docetaxel (Taxotere) is another drug that binds to the taxane site; and it is used to treat prostrate, brain, and lung tumors. Reference may be had, e.g., to articles by C. P. Belani et al. (“TAX 326 Study Group: First-line chemotherapy for NSCLC: an overview of relevant trials,” Lung Cancer, 38 (Suppl. 4): 13-19, 2002), and by F. V. Fosella et al. (“Second line chemotherapy for NSCLC: establishing a gold standard,” Lung Cancer, 38, 5-12, 2002).

The epothilones (such as BMS-247550, epothilones B and D) are other drugs that bind to the taxane site; they are used to treat paclitaxel-resistant tumors. References may be had, e.g., to articles by D. M. Bolag et al. (“Epothilones: a new class of microtubule-stabilizing agents with a taxol-like mechanism of action,” Cancer Res., 55: 2325-2333, 1995), by M. Wartmann et al. (“The biology and medicinal chemistiry of epothilones,” Curr. Med. Chem. Anti-Cancer Agents, 2: 123-148, 2002), by F. Y. Lee et al. (“BMS-247550: a novel epothilone analog with a mode of action similar to apcitaxel but possessing superior antitumour efficacy,” Clin. Cancer Res., 7: 1429-1437, 2001), and by K. Kamath et al. (“Suppression of microtubule dynamics by epothilone B in living MCF7 cells,” Cancer Res., 63: 6026-6031, 2003).

There are other microtubule binding sites disclosed in Table 1 of the 2004 Jordan et al. publication. Thus, e.g., it is disclosed that estramustine is used to treat prostrate cancer. Reference may be had, e.g., to articles by D. Panda et al. (“Stabilizatio of microtubule dynamics by estramustine by binding to a novel site in tubulin: a possible mechanistic basis forits antitumor action,” Proc. Nat. Acad. Sci USA94: 10560-10564, 1997), by O. Smaletz et al. (“Pilot study of epothilone B analog [BMS-247550] and estramustine phosphate in patients with progressive metastatic prostrate cancer following castration,” Ann. Oncol., 14: 1518-1524), by W. Kelly et al. (“Dose escalation study of intraveneous extramstine phosphate in combination with Paclitaxel and Carboplatin in patients with advanced prostate cancer,” Clin. Cancer Res. 9: 2098-2107, 2003), by G. Hudes et al. (“Phase 1 clinical and pharmacologic trial of intraveneous estramustine phosphate,” J. Clin. Oncol., 20: 1115-1127, 2002), and by B. Dahllof et al. (“Estramustine depolymerizes microtubules by binding to tubulin,” Cancer Res. 53, 4573-4581, 1993).

Referring again to the Jordan et al. article, and at page 256 thereof, the criticality of “highly dynamic microtubules” is discussed. It is disclosed that: “Mitosis in most cells progresses rapidly and the highly dynamic microtubules in the spindle are required for all stages of mitosis. First, for the timely and correct attachment of chromosomes at their kinetochoares to the spindle during prometaphase after nuclear-envelope breakdown . . . . Second, for the complex movements of the chromosomes that bring them to their properly aligned positons at the metaphase plate . . . . Last, for the synchronous separation of the chromosomes in anaphase and telophase after the metaphase . . . . During prometaphase, microtubules emanating from each of the two spindle poles make vast growing and shortening excursions, essentially probing the cytoplasm until they ‘find’ and become attached to chromosomes at their kinetocores . . . . Such microtubules must be able to grow for long distances . . . then shorten almost completely, then re-grow again, until they successfully become attached. The presence of a single chromosome that is unable to achieve a bipolar attachment to the spindle is sufficient to prevent a cell from transitioning to anaphase; the cell then remains blocked in a prometaphase/metaphase like state and eventually undergoes apoptosis (programmed cell death) . . . . We have found that suppression of microtubule dynamics by drugs such as paclitaxel (Taxol) and Vinca alkaloids seems to be a common mechanism by which these drugs block mitosis and kill tumour cells. Human osterosarcoma cells after inclubation with . . . paclitaxel and . . . vinflunine . . . are shown . . . . Many chromosomes are stuck at the spindle poles, unable to congress to the metaphase plate. At least one reason that cancer cells are relatively sensitive to these drugus compared to normal cells is that cancer cells divide more freuqenlty than normal cells and thereofore frequently pass though a stage of vulnerability to mitotic poisons.”

The anti-mitotic drugs may also interfere with “oscillations.” As is disclosed at page 257 of the Jordan et al. article, “During metaphase in the absence of drugs . . . the duplicated chromosomes, which are attached to the microtubules at their kinetohores, oscillate back and forth under high tension in the spindle equatorial region in concert with growth and shortening of the attached microtubles . . . . Superimposed on these oscillations, tubulin is continuously and rapidly added to microtubles at the kinetochores and is lost at the poles in a balanced fashion (that is, the microtubules treadmill) . . . . The oscillations are believed to be required for the proper functioning of the spindle. The absence of tension on the chromosomal kinetochores is also sufficient to block cell-cycle progress from metaphase to anaphase . . . . In apanphase . . . , microtubules that are attached to chromosomes must undergo a carefully regulated shortening at that same time that another propotion of spindle microtubles (the interpolar microtubules) lengthens.”

Anti-mitotic drugs interfere with these “microtubule dynamics” in different ways. As is disclosed at page 257 of the Jordan et al. article, “ . . . a large number of chemically diverse substances bind to soluble tubulin and/or directly to tubulin in the microtubules.” In one embodiment, the magnetic anti-mitotic drugs of this invention bind directly to soluble tubulin. In another embodiment, the magnetic anti-mitotic drugs of this invention binid to the polymerized tubulin in the microtubules.

As is also disclosed in the Jordan et al. article, “Most of these compounds are antimitotic agents and inhibit cell proliferation by actring on the polymerization dynamics of spindle microtubles, the rapid dynamics of which are essential to proper spindle function.” In one embodiment, the magnetic anti-mitotic compounds of this invention act on the polymerization dynamics of the spindle microtubules.

As is also disclosed in the Jordan et al. article, “The specific effects of individual microtubule-targeted drugs on the microtubule polymer mass and on the stability and dynamics of the microtubules are complex. Microtubule-targeted antimitoitic drugs are usually classified into two main groups. One group, known as the microtubule-destabilizing agents, inhibits microtubule polymerization at high concentrations . . . .” In one embodiment, the magnetic anti-mitotic compounds of this invention inihibit microtubule polymerization at high concentrations.

As is also disclosed in the Jordan et al. article, “The second main group is known as the microtubule stabilizing agents. These agents stimulate microtubule polymerization and include paclitaxel . . . docetaxel . . . the epothilones, discodermolide . . . and certain steroids . . . .” In one embodiment, the magnetic anti-mitotic compounds of this invention stimulate microtubule polymerization.

As is also disclosed in the Jordan et al. article, “The classification of drugs as microtubule ‘staiblizers’ or ‘destabilizers’ is overly simplistic . . . . The reason . . . is that drugs that increase or decrease microtubule polymerization at high concentrations powerfully suppress microtubule dynamics at 10-100 fold lower concentrations and, therefore, kinetically stabilize the microtubules, without changing the microtubule-polymer mass. In other words, the effects of the drugs on dynamics are often more powerful than their effects on polymer mass. It was previously thought that the effects of the two classes of drugs on microtubule-polymer mass were the most important actions resonsbile for their chemotherapeutic properties. However, the drugs would have to be given and maintained at very high dosage levels to act primarily and continuously on microtubule-polymer mass. It now seems that the most important action of these drugs is the suppression of spindle-microtubule dynamics, which results in the slowing or blocking of mitosis at the metaphase-anaphase transition and induction of apoptioic cell death.” In one embodiment, the magnetic properties of applicants' anti-mitotic compounds result in the slowing or blocking of mitosis at the metaphase-anaphase transition.

As is also disclosed in the Jordan et al. article, “The microtubule-targeted drugs affect microtubule dynamics in several different ways. To suppress microtubule dynamics for a significant time, the drugs must bind to and act directly on the microtubule. For example, a drug that suppresses the shortening rate at microtubule ends must bind directly to the microtubule, either at its end or along its length . . . many drugs also act on soluble tubulin, and the relatively ability of a given drug to bind to soluble tubulin or directly to the microtubule, and the location of the specific binding site in tubulin and the microtubule, greatly affect the response of the microtubule system to the drug.”

At page 258 of the Jordan et al. article, the mechanism by which Vinca alkaloids kills cancer cells is discussed. It is stated that: “Tubulin and microtubules are the main targets of the Vinca alkaloids . . . , which depolymerize microtubles and destroy mitotic spindles at high concentrations . . . , therefore leaving the dividing cancer cells blocked in mitosis with condensed chromosomes. At low but clinically relevant concentrations, vinbalstine does not depolymerize spindle microtubules, yet it powerfully blocks mitosis and cells die by apoposis. Studies form our laboratory . . . indicate that the block is due to suppression of microtubule dynamics rther than microtubule depolymerization . . . . Vinblastine binds to the beta-submit of tublin dimmers at a distict region called the Vinca-binding domain. Various other novel chemotherapeutic drugs also bind at this domain . . . . The binding of vinblastine to sulbue tubulin is rapid ad reversible . . . . Importantly, binding of vinblastine induces a conformational change in tubulin in connection with tubulin self-association . . . . The ability of vinlastine to increase the affinity of tubulin for itself probably has a key role in the ability of the drug to stabilize microtubules kinetically.”

The degree to which vinblastine binds to tubulin depends upon whether the tubulin is “exposed” or “buried.” As is also disclosed in the Jordan et al. article, “Vinblastine also binds directly to microtubules. In vitro, vinblastine binds to tubulin at the extreme microtubule ends . . . with very high affinity, but it binds with markedly reduced affinity to tubulin that is brued in the tubulin lattice . . . . Remarkably, the binding of one or two molecules of vinblastine per microtubule plus end is sufficient to reduce both treadmilling and dynamic instability by about 50 percent without causing appreciable microtubule depolymerization.”

By comparison, the taxanes bind poorly to soluble tubulin. As is also disclosed in the Jordan et al. article, “The taxanes bind poorly to soluble tubulin itself, but instead bind directly with high affinity to tubulin along the length of the microtubule . . . . The biding site for paclitaxel is in the beta-subunit, and its location, which is on the inside surface of the microtubule, is known with precision . . . . Paclitaxel is thought to gain access to its binding sites by diffusing through small openings in the microtubules or fluctuations in the microtubule lattice. Binding of paclitaxel to its site on the inside microtubule surface stalbilizes the microtubule and increases microtubule polymerization, presumably by inducing a conformational change in the tubulin that, by an unkown mechanism, increases its affinity for neighboring tubulin molecules.” In one preferred embodiment of this invention, a preferred magnetic anti-mitotic compound of the invention binds well to soluble tubulin.

Even relatlively small amounts of paclitaxel will stabilize the microtubules. As is disclosed in the Jordan et al. article, “There is one paclitaxel binding site on very molecule of tublin in a microtubule and the ability of paclitaxel to increase microtubule polymerization is associated with nearly 1:1 stoichiometric bind of paclitaxel to tubulin in microtubules So if a typical microtubule consists of approximately 10,000 tubulin molecules, then the ability of paclitaxel to increase microtubule polymerization requires the binding of about 5,000 packlitaxel molecules per microtubule. However, in contrast with the large number of molecules that are required to increase microtubule polymerization, we found that binding of a very small number of molecules stabilizes the dynamics of the microtubules without increasing microtubule polymerization.” Support for this statement in the article was a work by W. B. Derry et al., “Substoichiometric binding of taxol suppresses microtubule dynamics,” Biochemistry, 34: 2203-2211, 1995.

As is also disclosed in the Jordan et al. article, “ . . . just one paclitaxel molecule bound per several hundred tubulin molecules in a microtubule can reduce the rate of microtubule shortening by about 50 percent. Suppression of microtubule dynamics by paclitaxel leads to mitotic block in the absence of significant microtubule bundling.” Basis for this statement was an article by A. M. Yvon et al., “Taxol suppresses dynamics of individual microtubules in living human tumor cells,” Mol. Biol. Cell, 10:947-949, 1999. This Yvon et al. artricle was the “first demonstration that suppression of microtubule dynamics in living cells by low concentrations of paclitaxel correlates with mitotic block.”

As is also disclosed in the Jordan et al. article, “ . . . the suppression of spindle-microtubule dynamics prevents the dividing cancer cells from progressing from metaphase into anaphase and the cells eventually die by apoptosis.” As basis for this statement, articles were cited by M. A. Jordan et al. (“Mitotic block induced in HeLa cells by low concentrations of paclitaxel [Taxol] results in abnormal mitotic exit and apoptotic cell death,” Cancer Res., 56: 816-825, 1996), by Yvon et al. (“Taxol suppresses dynamics of individual microtubules in living human tumor cells, Mol. Biol. Cell, 10: 947-949, 1999), and by J. Kelling et al. (“Suppression of centromere dynamics by taxol in lving osteosarcoma cells,” Cancer Res., 63: 2794-2801, 2003).

The Jordan et al. article also discusses the mechanism by which colchicines exerts its anti-mitotic effects. At pages 260 et seq., it discloses that: “The interaction of colchicines with tubulin and microtubules presents yet another variation in the mechanisms by which microtubule-active drugs inhibit microtubule function. As with the Vinca alkaloids, colchicines depolymerizes microtubles at high concentrations and stabilizes microtubule dynamics at low concentrations. Colchicine inhibits microtubule polymerization substoichiometrically (at concentrations well below the concentration of tubulin that is free in solution . . . .” In support of this statement, the Jordan et al. article cites an article by L. Wilson et al. (in Microtubules [eds. J. S. Hymans et al.], 59-84 [Wiley-Liss, New York, N.Y., 1994]).

As is also disclosed in the Jordan et al. article, “ . . . colchicine itself does not bind directly to microtubule ends. Instead, it first binds to soluble tubulin, induces slow conformational changes in the tubulin and ultimately forms a poorly reversible final state tubulin-colchicine complex . . . which then copolymerizes into the microtubule ends in small numbers along with large numbers of free tubulin molecules.”

The Jordan et al. article discloses that the tubulin-colchicine complexes must bind more tightly to tublin that tubulin itself does, stating that: “Tubulin colchicines complexes might have a conformation that disrupts the microtubule lattice in a way that slows, but does not prevent, new tubulin addition. Importantly, the incorporated tubulin-colchicine complex must bind more tightly to its tubulin neighbors than tubulin itself does, so that the normal rate of tubulin dissociation is reduced.”

As is also disclosed in the Jordan et al. article, “So, despite the differences between the effects at high concentrations of the Vinca/colchicines-like drugs and the taxane-like drugs, nearly all of the microtubule-targeted antimitotic drugs stabilize microtubule dynamics at their lowest effective concentrations. Stabilization of microtubule dynamics correlates with blocking of the cell cycle at mitosis and in senstivie tumour cells, ultimately resulting in cell death by apoptosis. Therefore, the most potent mechanism of nearly all of the microtubule—targeted drugs seems to be the stabilization of dynamics of mitotic spindle microtubles.”

In one preferred embodiment of this invention, the antimitotic compounds of this invention inhibit the process of angiogenesis (the formation of new blood vessels). In another embodiment of this invention, the antimitotic compounds of this invention shut down the existing vasulature of tumors.

Prior art compositions that have these antivascular effects have been reported. Thus, as is disclosed at page 260 of the 2004 Jordan et al. article, ““The tumour vasculature is a relatively attractive new target for cancer therapy. The vasculature is easily accessible to blood-borne therapeutic agents, and tumour cells generally die rapidly unless they are supplied with oxygen and nutrients through the blood. There are two types of approaches to inhibiting vascular function. One . . . is the search for agents that inhibit the process of angiogenesis—the formation new blood vessels. However, more recently, the ability of several compounds, especially microtubule-targeted agents, to rapidly shout down existing turmour vasculature has been recognized . . . .” In support of this last statement, the Jordan et al. article cited an article by G. M. Tozer et al. on “The biologcy of the combretastatins as tumouor vascular targeting agents,” Int. J. Exp. Pathol., 83: 21-38 (2002).

As is also disclosed in the 2004 Jordan et al. article, “Since the late 1990s, the combestatins and N-acetylcolchicinol-O-phosphate, compounds that resemble colchicines and bind to the colchicines domain on tubulin, have undergone extensive development as antivascular agents . . . . When vascular targeting agents . . . are added to cultures of endothelial cells . . . , the microtubules rapidly depolymerize, the cells become round within minutes, undergo blebbing and detaching from the substrate, actin stress fibres form (presumably as a result of signaling from the depolymerizing microtubule cytoskeleton), and the cells die with no evidence of apoptosis.” As support for this latter statement, the 2004 Jordan et al. article cited a work by C. Kanthou et al., “The tumor vascular targeting agent combretastatin A-4 phosphate induces reorganization of the actin cytoskeleton and early membrane blebbing in human endothelial cells,” Blood, 99:2060-2069 (2002).

As is also disclosed in the 2004 Jordan et al. article, “The process of vascular shutdown can be observed in rats through windowed chambers that are implanted subcutaneously. This indicates that a primary and marked effect of vascular-targeting agents is an extremely rapid reduction of blood flow to the interior of solid tumours, often within 5 minutes of administering the drug to the aminal. Within 1 hour, the red-cell velocity might drop to less than 5 percent of the starting value.” As support for this statement, the 2004 Jordan et al. article cited a work by G. M. Tozer et al. on “Mechanisms associated with tumor vascular shut-down induced by combretastatin A-4 phosphate: intravital miscroscopy and measurement of vascular permeability,” Cancer Res., 61: 6413-6422 (2001).

The anti-vascular agents cause small blood vessels to disapper, blood flow to slow, red blood cells to aggregate in stacks or “rouleaux,” hemorrhaging from peripheral tumor vessels to occur, vascular permeability to increase, and the death of interior tumor cells by necrosis. See, e.g., an article by G. M. Tozer et al., “The Biology of the combretastatins as tumor vascular targeting agents,” Int. J. Exp. Pathol, 83: 21-38 (2002).

As is also disclosed in the 2004 Jordan et al. article, “ . . . the vascular-targeting aents that are now under development seem to damage tumour vasculature without significantly harming normal tissues . . . .” The Jordan et al. article, as support for this statement, cites work by V. E. Prise et al., reported in “The vascular response of tumor and normal tissues in the rat to the vascular targeting agent combretastatin A4 phosphate, at clinically relevant doses,” Int. J. Oncol. 21: 717-726 (2002). In one embodiment, the magnetic anti-mitotic compound of this invention damages tumors without significantly harming normal tissues.

As is also disclosed in the 2004 Jordan et al. article, “The source of this specificity is not known, but has been suggested to be attributable to differences between the mature vasculature of normal tissues and the immature or forming vasculature of tumors. There are suggestions that endothelial cells of immature vasculature could have a less well-developed actin cytoskeleton that might make the cells more susceptible to collapse.” The basis for this statement was an article by P. D. Davis et al., “ZD6126: A novel vascular-targeting agent that casues selective destruction of tumor vasculature,” Cancer Res. 62: 7247-7253 (2003).

As is also disclosed in the 2004 Jordan et al. article, “ . . . more sluggish or more variable blood flow in tumour vasculature might make the tumour vessels particularly susceptible to damaging agents. Differences in rates of endothelial-cell proliferation, in post-translational modifications of tubulin, and in interactions between actin and microtubules might also contribute to the specificity of vasclualr targeting agents.”

At page 261 of the 2004 Jordan et al. article, tumor sensitivity and resistance are discussed. It is disclosed that: “Among the most important unsolved questions about the antitumour activities of microtubule-targeted drugs concerns the basis of their tissue specificities and the basis for the development of drug resistance to these agents. For example, it is not known why paclitaxel is so effective against ovarian, mammary and lung tumours, but essentially ineffective against many other solid tumours, such as kidney or color carcinomas and various sarcomas. Similarly, for the Vinca alkaloids, it is unclear why they are frequently most effective against haematological cancers, but often ineffective against many solid tumors. There are clearly many determinants of sensitivity and resistance to antimitotic drugs, both at the level of the cells themselves and at the level of the pharmacological accessibility of the drugs to the tumour cells.” As authority for these statements, the 2004 Jordan et al. article cited work by C. Dumontet et al., “Mechanisms of action of and resistance to antitubulin agents: microtubule dynamics, drug transport, and cell death,” J. Clin. Oncol., 17: 1061-1070 (1999).

As is also disclosed in the 2004 Jordan et al. article, “the “ultimate failure or inherent resistance to chemotherapy with antimitotic drugs often results from overexpression of a class of membrane transporter proteins known as ABC-transporters (ATP-dependent drug efflux pumps or ATP-binding cassettes). These membrane pumps produce decreased intracellular drug levels and lead to cross-resistance (multidrug resistance) . . . to drugs of different chemical structures, such as paclitaxel and Vinca alkaloids. The first of many identified was P-glycoprotein, the product of the human MDRI gene.” As support for these statements, the 2004 Jordan et al. article cited work by S. V. Ambudkar et al., “P-glycoprotein: from genomics to mechanism,” Oncogene, 22: 7468-7485 (2003).

In one preferred embodiment, the magnetic anti-mitotic compound of this invention is not removed by these membrane pumps. It should be noted that, as is reported by the 2004 Jordan et al. article, “Considerable efforts are underway to understand these mechanisms of resitance, to develop P-glycoprotein inhibitors and to develop microtubule-targeted drugs that are not removed by these pumps. As authority for these statements, the 2004 Jordan et al. article cited works by S. V. Ambdukar et al. (see the citation in the preceding paragraph), by A. R. Safa (“Identification and characterization of the binding sites of P-glycoprotein for multidrug-resistance-related drugs and modulators,” Curr. Med. chem. Anti-Canc. Agents, 4: 1-17, 2004), by H. Thomas et al. (“Overcoming multidrug resistance in ancer: an udate on the clinical strategy of inhibiting P-glycoprotein,” Cancer Control, 10: 159-165, 2003), and by R. Geney et al. (“Overcoming multidrug resistance in taxane chemotherapy,” Clin. Chem. Lab. Med., 40: 918-925, 2002).

The 2004 Jordan et al. article discusses the role of specific tubulin isotypes in multidrug resitance. At page 262 of the article, it is stated that: “However, in addition, cells also have many microtubule-related mechanisms that confer resistance or determine intrinsic insensivity to antimitotic drugs.” As support for these statements, the Jordan et al. article cites an article by G. A. Orr et al. (“Mechanisms of taxol resistance related to microtubules,” Oncogene, 22: 7280-7295, 2003) which is a comprehensive review of microtubule-related mechanisms of paclitaxel resistance. The article also cites works by M. Kavallaris et al. (“Multiple microtubule alterations are associated with Vinca alkaloid resistance in human leukemia cells,” Cancer Res, 61: 5803-5809, 2001), by A. M. Minotti et al. (“Resistance to antimitotic drugs in Chinese hamster overay cells correlated with changes int eh level of polymerized tubulin,” J. Biol. Chem., 266: 3987-3994, 1991), by S. W. James et al. (A mutation in the . . . tubulin gene of Chlamydomonas reinhardtii confers resistance to anti-microtubule herbicides,” J. Cell Sci. 106: 209-218, 1993), by W. P. Lee et al. (“Purification and characterization of tublin form parental and vincristine-resistant HOB1 lymphoma cells,” Arch. Biochem. Biophys. 319: 498-503, 1995), by S. Ohta et al. (“Characterization of a taxol-resistant human small-cell lung cancer cell line,” Jpn. J. Cancer Res., 85: 290-297, 1994), and by N. M. Laing et al. (“Amplification of the ATP-binding cassette 2 transporter gene if unctionally linked with enhanced efflux of estramustine in overian carcinoma cells,” Cancer Res., 58: 1332-1337, 1998.)

In one preferred embodiment of this invention, the magnetic anti-mitotic compound of this invention binds to, and inactivates, a tubulin isotype that causes, or tends to cause, drug-resistance.

As is also disclosed in the 2004 Jordan et al. article, “Microtubule polymer levels and dynamics are regulated by a host of factors, including expression of regulatory proteins, post-translational modifications of tubulin and extression of different tubulin isotypes. The levels of each of these isotpypes differ among tissue and cell types, and there are numerous examples of changes in their levels that correlate with development of resistance of paclitaxel or Vinca alkaloids and other microtubule-targeted drugs.” In support of these statements, the Jordan et al. article cited works by C. M. Galmarini et al. (“Drug resistance associated with loss of p53 involves extensive alterations in microtubule composition and dynamics,” Br. J. Cancer, 88:1793-1799, 2003), by C. A. Burkart et al. (“The role of beta-tubulin isotpyes in resistance to antimitotic drugs,” Biochim. Biophys. Acta, 2: 01-09, 2001), by C. Dumontet et al. (“Resistance to microtubule-targeted cytotoxins in a K562 leukemia cell variant is associated with altered tubulin expression,” Elec. J. Oncol., 2: 33-44, 1999), by P. Giannakakou et al. (“A common pharmacophore for epothilone and taxanes: molecular basis for drug resistance conferred by tubulin mutations in human cancer cells, Proc. Natl. Acad. Sci USA, 97: 2904-2090, 2000), by A. Goncalves et al. (“Resistance to taxol in lung cancer cells associated with increased microtubule dynamics,” Proc. Natl. Acad. Sci USA, 98: 11737-11741, 2001), by M. Haber et al. (“Altered expression of M32, the class II beta-tubulin isotype, in a murine J774.2 cell line with a high level of taxol resistance,” J. Biol. Chem., 270: 31269-31275, 1995), by J. P. Jaffrezou et al. (“Novel mechanism of resistance to paclitaxel in human K562 leukemia cells by combined selection with PSC833,” Oncology Res., 7: 512-517, 1995), and by M. Kavallaris et al. (“Taxol-resistant epithelial ovarian tumors are associated with altered expression of specific beta-tubulin isotypes): J. Clin. Invest., 100: 1-12, 1997. In one embodiment, the “ . . . specific beta-tubulin isotypes” that are preferentially expressed by malignant cells are preferentially bound to (and inactivated) by the magnetic, anti-mitotic compound of this invention, as is more fully discussed elsewhere in this specification.

As is also disclosed in the 2004 Jordan et al. article, “ . . . subtle suppression of microtubule dynamics by paclitaxel, vinblastine or other antimitotic drugs, without any attendant change in the microtubule-polymer mass, prevents progress through the cell cycle from metaphase to anaphase in sensitive cells. Changes in microtubule dynamics can lead to altered sensitivity to microtubule-targeted drugs. In one well studied case of paclitaxel resistance, resistant and paclitaxel-depedent A549 lung cancer cells had inherently faster microtubule dynamics following withdrawal of paclitaxel than sensitive cells . . . .” As support for this statement, the article cited work by A. Goncalves et al., reported in “Resistance to taxol in lung cancer cells associated with increased microtubule dynamics,” Proc. Natl. Acad. Sci. USA, 98: 11737-11747, 2001.”

As is also disclosed in the 2004 Jordan et al. article, “In the absence of paclitaxel, the paclitaxel-resistant/dependent cells with the faster microtubule dynamics were unable to progress from metaphase to anaphase and their spindles became disorganized. So, these cells were resistant to paclitaxel and also dependent on paclitaxel to slow their dynamics and allow them to go through mitosis successfully. The inherent sensititivy of cells to subtle changes in microtubule dynamics means that there are numerous ways for cells to become resistant to microtubule-targeted drugs. In the case of the paclitaxel-resistant A549 cells discussed above, the mechanisms of increased dynamics seem to involve several changes. The resistant cells overexpress one of the isotypes of tubulin, BIII-tubulin.” As support for this last statement, the 2004 Jordan et al. article cited works by M. Kavallaris et al. (“Antisense oligonucleotides to class III beta-tubulin sensitive drug-resistant cells to taxol,” Br. J. Cancer, 80: 1020-1025, 1991), by L. A. Martello et al. (“Taxol and discodermolide represent a synergistic drug combination in human carcinoma cell lines,” Clin. Cancer Res., 6: 1978-1987, 2000), and another article by Martello et al. (“Elevated levels of microtubule-destabilizing factors in a taxol-resistant A549 cell line with a alpha-tubulin mutation,” Cancer Res., 63: 1207-1213, 2003. In one embodiment of this invention, the anti-mitotic compound of this invention is used to bind with, and inactivate, the beta-tubulin isotype(s) expressed by the drug-resistant cancer cells.

As is also disclosed in the 2004 Jordan et al. article. “In addition, they have a heterozygous point mutation in alpha-tubulin and they overexpress the ative form of the microtubule-destabilizing protein stahmin and the inactive form of the putative microtubule stabiling protein MAP 4 . . . . ”

As is also disclosed in the 2004 Jordan et al. article, “ . . . drug resistance might involve some of the ther forms of tubulin . . . that associate with the centrosomes in intrphase and with the spindle poles in mitotic cells.” In one embodiment of this invention, the anti-mitotic compound of this invention binds to, and inactivates, one or more of these other forms of tubulin.

As is also disclosed in the 2004 Jordan et al. article. “The fact that antimitotic drugs bind to many diverse sites on tubulin and microtubles mean that clinical combinations of two or more of these drugs have the potential to improve efficiency and reduce the side effects of therapy.” In one embodiment of this invention, the actions of two or more separate chemotherapeutic agents are combined into one compound or composition. In another embodiment, the anti-mitotic compound of this invention is administered with another chemotherapeutic agent, prior to the administration of another chemotherapeutic agent, or after the administration of another chemotherapeutic agent. This embodiment is discussed elsewhere in this specification.

As is also disclosed in the 2004 Jordan et al. article, “The discovery of the synergistm of paclitaxel with discodermolide is particularly interesting, as both drugs bind to the same or overlapping sites on tubulin or microtubules.” In one embodiment, the magnetic, anti-mitotic compound of this invention binds to the same or averlapping sites on tubulin or microtubules as does paclitaxel.

Many of the matters disclosed in the 2004 Jordan et al. article regarding tubulin isotype are also disclosed in the patent literature.

By way of illustration, U.S. Pat. No. 5,888,818, the entire disclosure of which is hereby incorporated by reference into this specification, claims “An isolated DNA encoding an .alpha.- or .gamma.-tubulin, which tubulin is resistant to an anti-tubulin agent selected from the group consisting of dinitroanaline, phosphorothioamidate and chlorthal dimethyl, the resistant tubulin comprising a non-polar amino acid instead of a threonine residue at a position corresponding to that depicted as position 239, 237, or 240 in Table 2.” At columns 1 et seq. of such patent, an excellent discussion of microtubules and tubulin isotypes is presented.

Thus, as is disclosed in U.S. Pat. No. 5,888,818, “Almost all eukaryotic cells contain microtubules which comprise a major component of the network of proteinaceous filaments known as the cytoskeleton. Microtubules thereby participate in the control of cell shape and intracellular transport. They are also the principal constituent of mitotic and meiotic spindles, cilia and flagella. In plants, microtubules have additional specialized roles in cell division and cell expansion during development.”

As is also disclosed in U.S. Pat. No. 5,888,818, “In terms of their composition, microtubules are proteinaceous hollow rods with a diameter of approximately 24 nm and highly variable length. They are assembled from heterodimer subunits of an .alpha.-tubulin and a β-tubulin polypeptide, each with a molecular weight of approximately 50,000. Both polypeptides are highly flexible globular proteins (approximately 445 amino acids), each with a predicted 25% .alpha. helical and 40% β-pleated sheet content. In addition to the two major forms (.alpha.- and β-tubulin), there is a rare .gamma.-tubulin form which does not appear to participate directly in the formation of microtubule structure, but rather it may function in the initiation of microtubule structure.”

As is also disclosed in U.S. Pat. No. 5,888,818, “In all organisms, the multiple .alpha.- and β-tubulin polypeptides are encoded by corresponding families of alpha.- and β-tubulin genes, which are located in the nuclear genome. Many such genes (or corresponding cDNAs) have been isolated and sequenced. For example, maize has approximately 6 alpha.-tubulin genes and approximately 8 β-tubulin genes dispersed over the genome (Villemur et al, 1992, 34th Maize Genetics Symposium). Some of the .alpha.-tubulin genes from maize have been cloned and sequenced (Montoliu et al, 1989, Plant Mol Biol, 14, 1-15; Montoliu et al, 1990, Gene, 94, 201-207; Villemur et al, 1992, J Mol Biol, 227:81-96), as have some of the β-tubulin genes (Hussey et al, 1990, Plant Mol Biol, 15, 957-972). Comparison of amino acid sequences of the three documented maize .alpha.-tubulins indicates they have 93% homology. Maize β-tubulins exhibit 38% identity with these .alpha.-tubulins. In segments of divergence between the .alpha.- and β-tubulin amino acid sequences, homology ranges from 13% to 17%. Homology between the three .alpha.-tubulin amino acid sequences within these same .alpha.-/β-divergence regions ranges from 77% to 96%.”

As is also disclosed in U.S. Pat. No. 5,888,818, “Sequence information on the various tubulin forms shows that throughout evolution the protein domains involved in polymerization have been highly conserved, and interspecies amino acid sequence homology is generally high. For example, the four β-tubulin isotypes in human are identical with their counterparts in mouse. There is 82-90% homology between mammalian neuronal or constitutively expressed tubulins and algal, protozoan and slime mould tubulins. Considering plant sequences in more detail, there are long stretches in which the amino acid sequence of all the alpha.- and β-tubulins are identical (Silflow et al, 1987, Developmental Genetics, 8, 435-460). For example, the 35 amino acids in positions 401-435 are identical in all plant alpha.-tubulins, as are the 41 amino acids in the region between positions 240 and 281 in the plant β-tubulins. Conservation of amino acid residues is approximately 40% between the alpha.- and β-tubulin families, and 85-90% within each of the alpha.- and β-tubulin families. It should be noted that in general, most .alpha.-tubulins are 1 to 5 residues larger that the β-tubulins.”

U.S. Pat. No. 5,888,818 then goes on to discuss anti-tubulin agents, stating that: “The economic interest of tubulins lies in the effect of certain agents which interfere with tubulin structure and/or function. Such agents (including non-chemical stresses) are hereinafter referred to as ‘anti-tubulin agents’ as they share a similar type of mode of action. Extreme conditions are known to destabilize the tubulins and/or microtubules. Such conditions include cold, pressure and certain chemicals. For example, Correia (1991, Pharmac Ther, 52:127-147) describes .alpha.- and β-tubulin interactions, microtubule assembly and drugs affecting their stability. Some anti-tubulin agents are often called ‘spindle poisons’ or ‘antimitotic agents’ because they cause disassembly of microtubules which constitute the mitotic spindle. For at least one hundred years, it has been known that certain chemical agents arrest mammalian cells in mitosis, and of these agents the best known is colchicine which was shown in the mid-1960s to inhibit mitosis by binding to tubulin. Many of these anti-tubulin agents have since found widespread use as cancer therapeutic agents (eg vincristine, vinblastine, podophyllotoxin), estrogenic drugs, anti-fungal agents (eg griseofulvin), antihelminthics (eg the benzimidazoles) and herbicides (eg the dinitroanilines). Indeed some of the specific agents have uses against more than one class of organism. For example, the dinitroaniline herbicide trifluralin has recently been shown to inhibit the proliferation and differentiation of the parasitic protozoan Leishmania (Chan and Fong, 1990, Science, 249:924-926).” Thus, as is apparent from this teaching, the magnetic, anti-mitotic drugs disclosed in this specification may be used not only to treat cancer but also as “ . . . estrogenic drugs, anti-fungal agents . . . , antihelminthics . . . and herbicides . . . .”

As is also disclosed in U.S. Pat. No. 5,888,818, “The dinitroaniline herbicides may be considered as an example of one group of anti-tubulin agents. Dinitroaniline herbicides are widely used to control weeds in arable crops, primarily for grass control in dicotyledonous crops such as cotton and soya. Such herbicides include trifluralin, oryzalin, pendimethalin, ethalfluralin and others. The herbicidally active members of the dinitroaniline family exhibit a common mode of action on susceptible plants. For example, dinitroaniline herbicides disrupt the mitotic spindle in the meristems of susceptible plants, and thereby prevent shoot and root elongation (Vaughn K C and Lehnen L P, 1991, Weed Sci, 39:450-457). The molecular target for dinitroaniline herbicides is believed to be tubulin proteins which are the principle constituents of microtubules (Strachan and Hess, 1983, Pestic Biochem Physiology, 20, 141-150; Morejohn et al, 1987, Planta, 172, 252-264).”

As is also disclosed in U.S. Pat. No. 5,888,818, “The extensive interest in anti-tubulin agents in many branches of science has been accompanied by the identification of several mutants shown to resist the action of such agents (Oakley B R, 1985, Can J Blochem Cell Biol, 63:479-488). Several of these mutants have been shown to contain modified alpha.- or β-tubulin genes, but to date the only resistant mutants to be fully characterised and sequenced are those in β-tubulin. For example, colchicine resistance in mammalian cell lines is closely associated with modified β-tubulin polypeptides (Cabral et al, 1980, Cell, 20, 29-36); resistance to benzimidazole fungicides has been attributed to a modified β-tubulin gene, for example in yeast (Thomas et al, 1985, Genetics, 112, 715-734) and Aspergillus (Jung et al, 1992, Cell Motility and the Cytoskeleton, 22:170-174); some benzimidazole resistant forms of nematode are known; and dinitroaniline-resistant Chlamydomonas mutants possess a modified β-tubulin gene (Lee and Huang, 1990, Plant Cell, 2, 1051-1057). Some of these mutants, although resistant to one anti-tubulin agent, also show increased susceptibility to other anti-tubulin agents (such as cold stress).” As is also discussed elsewhere in this, and in one preferred embodiment, the anti-mitotic compounds and/or compositions of this invention are adapted to bind one or more of the tubulin isotypes expressed by such mutants.

As is also disclosed in U.S. Pat. No. 5,888,818, “Among certain weed species, some biotypes have evolved resistance to dinitroaniline herbicides. Three examples of species in which dinitroaniline resistant (R) biotypes have emerged are goosegrass, Eleusine indica (Mudge et al, 1984, Weed Sci, 32, 591-594); green foxtail, Setaria viridis (Morrison et al, 1989, Weed Technol, 3, 554-551); and Amaranthus palmeri (Gossett et al, 1992, Weed Technology, 6:587-591). These resistant (R) biotypes emerged following selective pressure exerted by repeated application of trifluralin. A range of resistant biotypes of each species exists but the nature and source of the resistance trait is unclear and the biotypes are genetically undefined. The R biotypes of these species exhibit cross-resistance to a wide range of dinitroaniline herbicides, including oryzalin, pendimethalin and ethalfluralin. All dinitroaniline herbicides have a similar mode of action and are therefore believed to share a common target site. Many of the R biotypes are also cross-resistant to other herbicide groups such as the phosphorothioamidates, which include amiprophos-methyl and butamifos, or chlorthal-dimethyl. The phenomenon of cross-resistance exhibited by resistant biotypes strongly indicates that the herbicide resistance trait is a consequence of a modified target site. In addition, the resistant biotypes appear to have no competitive disadvantage as they grow vigorously and can withstand various stresses (such as cold).” To the extent that the drug resistant trait is “ . . . a consequence of a modified target site . . . ,” and in one preferred embodiment, the magnetic anti-mitotoic compounds of this invention are adapted to preferentially bind to such modified target site.

As is also disclosed in U.S. Pat. No. 5,888,818, “It has not been previously shown which specific gene is modified in Eleusine indica or Setaria viridis to confer the dinitroaniline resistance trait. Research by K. C. Vaughn and M. A. Vaughn (American Chemical Society Symposium Series, 1989, 364-375) showed an apparent alteration in the electrophoretic properties of β-tubulin present in an R biotype of Eleusine indica, and suggested dinitroaniline resistance results from the presence of a modified β-tubulin polypeptide. The results of recent work by Waldin, Ellis and Hussey (1992, Planta, 188:258-264) provide no evidence that dinitroaniline herbicide resistance is associated with an electrophoretically modified β-tubulin polypeptide in the resistant biotypes of Eleusine indicaor Setaria viridis which were studied.” In one preferred embodiment of this invention, the magnetic anti-mitotic agent of this invention is adapted to bind to a target site on a beta-tubulin polypeptide.

U.S. Pat. No. 6,306,615, the entire disclosure of which is hereby incorporated by reference into this specification, claims a detection method for identifying modified beta-tubulin isotypes. Thus, e.g., claim 17 of this patent discloses: “17. A method of monitoring the amount of a tubulin modified at a cysteine residue at amino acid position 239 in a patient treated with a sulfhydryl or a disulfide tubulin modifying agent, the method comprising the steps of: (a) providing a sample from the patient treated with the tubulin modifying agent; (b) contacting the sample with an antibody that specifically binds to the tubulin modified at a cysteine residue at amino acid position 239; and (c) determining the amount of the tubulin modified at a cysteine residue at amino acid position 239 in the patient sample by detecting the antibody and comparing the amount of antibody detected in the patient sample to a standard curve, thereby monitoring the amount of the tubulin modified at a cysteine residue at amino acid position 239 in the patient.”

As is also disclosed in U.S. Pat. No. 6,306,615, “Microtubules are composed of .alpha./β-tubulin heterodimers and constitute a crucial component of the cell cytoskeleton. Furthermore, microtubules play a pivotal role during cell division, in particular when the replicated chromosomes are separated during mitosis. Interference with the ability to form microtubules from .alpha./β-tubulin heterodimeric subunits generally leads to cell cycle arrest. This event can, in certain cases, induce programmed cell death. Thus, natural products and organic compounds that interfere with microtubule formation have been used successfully as chemotherapeutic agents in the treatment of various human cancers.”

As is also disclosed in U.S. Pat. No. 6,306,615, “Pentafluorophenylsulfonamidobenzenes and related sulfhydryl and disulfide modifying agents (see, e.g., compound 1; 2-fluoro-1-methoxy-4-pentafluorophenylsulfonamidobenzene; . . . prevent microtubule formation by selectively covalently modifying β-tubulin. For example, compound 1 does not covalently modify all of the five known β-tubulin isotypes. Instead, binding is restricted to those β-tubulin isotypes that have a cysteine residue at amino acid position 239 in β-tubulin. Such isotypes include beta-1, beta-2, and beta-4. The other two isotypes (beta-3 and beta-5) have a serine residue at this particular position (Shan et al., Proc. Nat'l Acad. Sci USA 96:5686-5691 (1999)). It is notable that no other cellular proteins are modified by compound 1.” In one embodiment of this invention, the anti-mitotic compound of this invention selectively covalently modifies certain beta-tubulin isotypes but does not covalently modify other proteins.

U.S. Pat. No. 6,362,321. the entire disclosure of which is hereby incorporated by reference into this specification, discusses taxol-resistant cancer cell lines. At column 1 of this patent, it is disclosed that: “Many of the most common carcinomas, including breast and ovarian cancer, are initially relatively sensitive to a wide variety chemotherapy agents. However, acquired drug resistance phenotype typically occurs after months or years of exposure to chemotherapy. Determining the molecular basis of drug resistance may offer opportunities for improved diagnostic and therapeutic strategies.”

As is also disclosed in U.S. Pat. No. 6,362,32, “Taxol is a natural product derived from the bark of Taxus brevafolio (Pacific yew). Taxol inhibits microtubule depolymerization during mitosis and results in subsequent cell death. Taxol displays a broad spectrum of tumorcidal activity including against breast, ovary and lung cancer (McGuire et al., 1996, N. Engld. J. Med. 334:1-6; and Johnson et al., 1996, J. Clin. Ocol. 14:2054-2060). While taxol is often effective in treatment of these malignancies, it is usually not curative because of eventual development of taxol resistance. Cellular resistance to taxol may include mechanisms such as enhanced expression of P-glycoprotein and alterations in tubulin structure through gene mutations in the β chain or changes in the ratio of tubulin isomers within the polymerized microtubule (Wahl et al., 1996, Nature Medicine 2:72-79; Horwitz et al., 1993, Natl. Cancer Inst. 15:55-61; Haber et al., 1995, J. Biol. Chem. 270:31269-31275; and Giannakakou et al., 1997, J. Biol. Chem. 272:17118-17125). Some tumors acquires taxol resistance through unknown mechanisms.

International publication WO 02/36603 A2, the entire disclosure of which is hereby incorporated by reference into this specification, discloses nucleic acid molecules comprising a nucleotide sequence encoding a tubulin molecule. At pages 1 et seq. of this patent document, it is disclosed that: “Microtubules are essential to the eucaryotic cell due as they are involved in many processes and functions such as, e.g., being components of the cytoskeleton, of the centrioles and ciliums and in the formation of spindle fibres during mitosis. The constituents of microtubules are heterodimers consisting of one alpha-tubulin molecule and one beta-tubulin molecule. These two related self-associating 50 kDa proteins are encoded by a multigen family. The various members of this multigen family are dispersed all over the human genorne. Both alpha-tubulin and beta-tubulin are most likely to originate from a common ancestor as their amino acid sequence shows a homology of up to 50%. In man there are at least 15 genes or pseudogenes for tubulin.

As is also disclosed in International Publication WO 02/36603, “The conservation of structure and regulatory functions among the beta-tubulin genes in three vertebrate species (chicken, mouse and human) allowed the identification of and categorization into six major classes of beta-tubulin polypeptide isotypes on the basis of their variable carboxyterminal ends. The specific, highly variable 15 carboxyterminal amino acids are very conserved among the various species. Beta-tubulins of categories I, II, and IV are closely related differing only 2-4% in contrast to categories III, V and VI which differ in 8-16% of amino acid positions [Sullivan K. F., 1988, Ann. Rev. Cell Biol. 4: 687-716].

As is also disclosed in International Publication WO 02/36603, “Also the expression pattern is very similar between the various species as can be taken from the following table [Sullivan K. F., 1988, Arm. Rev. Cell Biol. 4: 687-716] which comprises the respective human members of each class . . . . The C terminal end of the beta-tubulins starting from amino acid 430 is regarded as highly variable between the various classes. Additionally, the members of the same class seem to be very conserved between the various species.”

As is also disclosed in International Publication WO 02/36603, “As tubulin molecules are involved in many processes and form part of many structures in the eucaryotic cell, they are possible targets for pharmaceutically active compounds. As tubulin is more particularly the main structural component of the microtubules it may act as point of attack for anticancer drugs such as vinblastin, colchicin, estramustin and taxol which interfere with microtubule function. The mode of action is such that cytostatic agents such as the ones mentioned above, bind to the carboxyterminal end the beta-tubulin which upon such binding undergoes a conformational change. For example, Kavallaris et al. [Kavallaris et al. 1997, J. Clin. Invest. 100: 1282-1293] reported a change in the expression of of specific beta-tubulin isotypes (class I, II, III, and IVa) in taxol resistant epithelial ovarian tumor. It was concluded that these tubulins are involved in the formation of the taxol resistence. Also a high expression of class III beta—tubulins was found in some forms of lung, cancer suggesting that this isotype may be used as a diagnostic marker.”

As is also disclosed in International Publication WO 02/36603, “The problem underlying the present invention was to provide the means to further characterize the various tubulins present in eucaryotic cells. A further problem underlying the present invention was to provide the means to extend possible screening programs for cytostatic agents to other isotypes of human beta-tubulins. This problem is solved in a first aspect by a nucleic acid molecule comprising a nucleotide sequence encoding a tubulin molecule, wherein said nucleic acid molecule comprises the sequence according to SEQ. ID. No. I This problem is ‘solved in a second aspect by a nucleic acid molecule comprising a nucleotide sequence encoding a tubulin molecule, wherein said nucleic acid molecule comprises the sequence according to SEQ. ID. No. 2.”

Published U.S. patent application 2002/0106705, the entire disclosure of which is hereby incorporated by reference into this specification, describes a method for detecting a modified beta-tubulin isotype. Claim 1 of this patent, which is typical, describes: “A method of detecting in a sample a P-tubulin isotype modified at cysteine residue 239, the method comprising the steps of: (a) providing a sample treated with a β-tubulin modifying agent; (b) contacting the sample with an antibody that specifically binds to a β-tubulin isotype modified at cysteine residue 239; and (c) determining whether the sample contains a modified β-tubulin isotype by detecting the antibody.” This patent discloses that: “Microtubules are composed of α/β-tubulin heterodimers and constitute a crucial component of the cell cytoskeleton. Furthermore, microtubules play a pivotal role during cell division, in particular when the replicated chromosomes are separated during mitosis. Interference with the ability to form microtubules from α/β-tubulin heterodimeric subunits generally leads to cell cycle arrest. This event can, in certain cases, induce programmed cell death. Thus, natural products and organic compounds that interfere with microtubule formation have been used successfully as chemotherapeutic agents in the treatment of various human cancers.”

Published United States paent application 2002/0106705 also discloses that: “Pentafluorophenylsulfonamidobenzenes and related sulfhydryl and disulfide modifying agents (see, e.g., compound 1; 2-fluoro-1-methoxy-4-pentafluorophenylsulfonamidobenzene . . . prevent microtubule formation by selectively covalently modifying β-tubulin. For example, compound 1 does not covalently modify all of the five known β-tubulin isotypes. Instead, binding is restricted to those β-tubulin isotypes that have a cysteine residue at amino acid position 239 in β-tubulin. Such isotypes include β1, β2 and β4-tubulin. The other two isotypes (β3 and β5) have a serine residue at this particular position (Shan et al., Proc. Nat'l Acad. Sci USA 96:5686-5691 (1999)). It is notable that no other cellular proteins are modified by compound 1.”

Published United States paent application 2002/0106705 relates primarily to a “ . . . a β-tubulin isotype modified at cysteine residue 239 . . . ” Thus, at page 3 of this published patent application, in defining a “beta-tubulin modifying agent,” it describes such agent as follows: “A “β-tubulin modifying agent” refers to an agent that has the ability to specifically react with an amino acid residue of β-tubulin, preferably a cysteine, more preferably the cysteine residue at position 239 of a β-tubulin isotype such as β1- β2- or β4-tubulin and antigenic fragments thereof comprising the residue, preferably cysteine 239. The β-tubulin modifying agent of the invention can be, e.g., any sulfhydryl or disulfide modifying agent known to those of skill in the art that has the ability to react with the sulfur group on a cysteine residue, preferably cysteine residue 239 of a β-tubulin isotype. Preferably, the β-tubulin modifying agents are substituted benzene compounds, pentafluorobenzenesulfonamides, arylsulfonanilide phosphates, and derivatives, analogs, and substituted compounds thereof (see, e.g., U.S. Pat. No. 5,880,151; PCT 97/02926; PCT 97/12720; PCT 98/16781; PCT 99/13759; and PCT 99/16032, herein incorporated by reference; see also Pierce Catalogue, 1999/2000, and Means, Chemical Modification of Proteins). In one embodiment, the agent is 2-fluoro-1-methoxy-4-pentafluorophenylsulfonamidobenzene (compound 1; FIG. 1C). Modification of a P-tubulin isotype at an amino acid residue, e.g., cysteine 239, by an agent can be tested by treating a β-tubulin peptide, described herein, with the putative agent, followed by, e.g., elemental analysis for a halogen, e.g., fluorine, reverse phase HPLC, NMR, or sequencing and HPLC mass spectrometry. Optionally compound 1 described herein can be used as a positive control. Similarly, an α-tubulin modifying agent refers to an agent having the ability to specifically modify an amino acid residue of an α-tubulin.”

U.S. Pat. No. 6,541,509, the entire disclosure of which is hereby incorporated by reference into this specification, discloses a “method for treating neoplasis using combination chemotherapy.” Claim 1 of this patent describes: “A method of treating neoplasia in a subject in need of treatment, comprising administering to the subject an amount of paclitaxel effective to treat the neoplasia, in combination with an amount of discodermolide effective to treat the neoplasia, wherein a synergistic antineoplastic effect results.” At column 6 of this patent, the patentees discuss how to determine synergy between two drugs. They state that: One measure of synergy between two drugs is the combination index (CI) method of Chou and Talalay [37], which is based on the median-effect principle. This method calculates the degree of synergy, additivity, or antagonism between two drugs at various levels of cytotoxicity. Where the CI value is less than 1, there is synergy between the two drugs. Where the CI value is 1, there is an additive effect, but no synergistic effect. CI values greater than 1 indicate antagonism. The smaller the CI value, the greater the synergistic effect. Another measurement of synergy is the fractional inhibitory concentration (FIC) [48]. This fractional value is determined by expressing the IC50 of a drug acting in combination, as a function of the IC50 of the drug acting alone. For two interacting drugs, the sum of the FIC value for each drug represents the measure of synergistic interaction. Where the FIC is less than 1, there is synergy between the two drugs. An FIC value of 1 indicates an additive effect. The smaller the FIC value, the greater the synergistic interaction. In the method of the present invention, combination therapy using paclitaxel and discodermolide preferably results in an antineoplastic effect that is greater than additive, as determined by any of the measures of synergy known in the art.” The cited Chou et al. reference is an entited “Quantitative analysis of dose effect relationships: the combined effect of multiple drugs or enzyme inhibitors,” Adv. Enzyme Regul., 11:27-56 (1984). The cited “reference 48 is an article by Hall et al., “The fractional inhibitory concentration (FIC) as a measure of synergy,” J. Antimicrob. Chemother., 11(5):427-433 (1983).

Claim 8 of U.S. Pat. No. 6,541,509 describes “A synergistic combination of antineoplastic agents, comprising an effective antimenoplastic amount of paclitaxel and an effective antineoplastic amount of discodermolide.” As one embodiment of the instant invention, applicants claims: A synergistic combination of antineoplastic agents, comprising an effective antimenoplastic amount of paclitaxel and an effective antineoplastic amount of the preferred, magnetic anti-mitotic compound of this inventon. Thus, the process of such U.S. Pat. No. 6,541,509 may be adapted to use the magnetic compound of this invention instead of discodermolide.

As is disclosed in U.S. Pat. No. 6,541,509, “The present invention provides a method of treating neoplasia in a subject in need of treatment. As used herein, ‘neoplasia’ refers to the uncontrolled and progressive multiplication of cells under conditions that would not elicit, or would cause cessation of, multiplication of normal cells. Neoplasia results in the formation of a ‘neoplasm’, which is defined herein to mean any new and abnormal growth, particularly a new growth of tissue, in which the growth is uncontrolled and progressive. Malignant neoplasms are distinguished from benign in that the former show a greater degree of anaplasia, or loss of differentiation and orientation of cells, and have the properties of invasion and metastasis. Thus, neoplasia includes ‘cancer’, which herein refers to a proliferation of cells having the unique trait of loss of normal controls, resulting in unregulated growth, lack of differentiation, local tissue invasion, and metastasis.” As support for this statement, the patent cited a work by Beers and Berkow (eds.), The Merck Manual of Diagnosis and Therapy, 17^{th} edition (Whitehouse Station, N.J.; Merck Research Laboratories, 1999, 973-974, 976, 986, and 991).

As is also disclosed in U.S. Pat. No. 6,541,509, “ . . . neoplasia is treated in a subject in need of treatment by administering to the subject an amount of paclitaxel effective to treat the neoplasia, in combination with an amount of discodermolide effective to treat the neoplasia, wherein a synergistic antineoplastic effect results. The subject is preferably a mammal (e.g., humans, domestic animals, and commercial animals, including cows, dogs, monkeys, mice, pigs, and rats), and is most preferably a human.” In the embodiment described in this specification, the magnetic compound of this invention replaces discomdermolide.

As is also disclosed in U.S. Pat. No. 6,541,509, “ . . . ‘paclitaxel’ refers to paclitaxel and analogues and derivatives thereof, including, for example, a natural or synthetic functional variant of paclitaxel which has paclitaxel biological activity, as well as a fragment of paclitaxel having paclitaxel biological activity. As further used herein, the term “paclitaxel biological activity” refers to paclitaxel activity which interferes with cellular mitosis by affecting microtubule formation and/or action, thereby producing antimitotic and antineoplastic effects. Furthermore, as used herein, ‘antineoplastic’ refers to the ability to inhibit or prevent the development or spread of a neoplasm, and to limit, suspend, terminate, or otherwise control the maturation and proliferation of cells in a neoplasm.”

As is also disclosed in U.S. Pat. No. 6,541,509, “Methods of preparing paclitaxel and its analogues and derivatives are well-known in the art, and are described, for example, in U.S. Pat. Nos. 5,569,729; 5,565,478; 5,530,020; 5,527,924; 5,484,809; 5,475,120; 5,440,057; and 5,296,506. Paclitaxel and its analogues and derivatives are also available commercially. Synthetic paclitaxel, for example, can be obtained from Bristol-Myers Squibb Company, Oncology Division (Princeton, N.J.), under the registered trademark Taxol. Taxol for injection may be obtained in a single-dose vial, having a concentration of 30 mg/5 mL (6 mg/mL per 5 mL) [47]. Taxol and its analogues and derivatives have been used successfully to treat leukemias and tumors. In particular, Taxol is useful in the treatment of breast, lung, and ovarian cancers. Discodermolide and its analogues and derivatives can be isolated from extracts of the marine sponge, Discodermia dissoluta, as described, for example, in U.S. Pat. Nos. 5,010,099 and 4,939,168. Discodermolide and its analogues and derivatives also may be synthesized, as described, for example, in U.S. Pat. No. 6,096,904. Moreover, both paclitaxel and discodermolide may be synthesized in accordance with known organic chemistry procedures [46] that are readily understood by one skilled in the art.”

As is also disclosed in U.S. Pat. No. 6,541,509, “In the method of the present invention, an amount of paclitaxel or discodermolide that is ‘effective to treat the neoplasia’ is an amount that is effective to ameliorate or minimize the clinical impairment or symptoms of the neoplasia, in either a single or multiple dose. For example, the clinical impairment or symptoms of the neoplasia may be ameliorated or minimized by diminishing any pain or discomfort suffered by the subject; by extending the survival of the subject beyond that which would otherwise be expected in the absence of such treatment; by inhibiting or preventing the development or spread of the neoplasm; or by limiting, suspending, terminating, or otherwise controlling the maturation and proliferation of cells in the neoplasm. For example, doses of paclitaxel (Taxol) administered intraperitoneally may be between 1 and 10 mg/kg, and doses administered intravenously may be between 1 and 3 mg/kg, or between 135 mg/m2 and 200 mg/m2. However, the amounts of paclitaxel and discodermolide effective to treat neoplasia in a subject in need of treatment will vary depending on the particular factors of each case, including the type of neoplasm, the stage of neoplasia, the subject's weight, the severity of the subject's condition, and the method of administration. These amounts can be readily determined by the skilled artisan.”

As is also disclosed in U.S. Pat. No. 6,541,509, “The method of the present invention may be used to treat neoplasia in a subject in need of treatment. Neoplasias for which the present invention will be particularly useful include, without limitation, carcinomas, particularly those of the bladder, breast, cervix, colon, head, kidney, lung, neck, ovary, prostate, and stomach; lymphocytic leukemias, particularly acute lymphoblastic leukemia and chronic lymphocytic leukemia; myeloid leukemias, particularly acute monocytic leukemia, acute promyelocytic leukemia, and chronic myelocytic leukemia; malignant Iymphomas, particularly Burkitt's lymphoma and Non-Hodgkin's lymphoma; malignant melanomas; myeloproliferative diseases; sarcomas, particularly Ewing's sarcoma, hemangiosarcoma, Kaposi's sarcoma, liposarcoma, peripheral neuroepithelioma, and synovial sarcoma; and mixed types of neoplasias, particularly carcinosarcoma and Hodgkin's disease [45]. Preferably, the method of the present invention is used to treat breast cancer, colon cancer, leukemia, lung cancer, malignant melanoma, ovarian cancer, or prostate cancer.” The aforementioned neoplasias may also be treated by the process of the instant invention.

As is also disclosed in U.S. Pat. No. 6,541,509, “In the method of the present invention, paclitaxel is administered to a subject in combination with discodermolide, such that a synergistic antineoplastic effect is produced. A ‘synergistic antineoplastic effect’ refers to a greater-than-additive antineoplastic effect which is produced by a combination of two drugs, and which exceeds that which would otherwise result from individual administration of either drug alone. Administration of paclitaxel in combination with discodermolide unexpectedly results in a synergistic antineoplastic effect by providing greater efficacy than would result from use of either of the antineoplastic agents alone. Discodermolide enhances paclitaxel's effects. Therefore, lower doses of one or both of the antineoplastic agents may be used in treating neoplasias, resulting in increased therapeutic efficacy and decreased side-effects.” As will be apparent, in applicants' invention the discodermolide is replaced by the magnetic anti-mitotic compound described in this specification.

As is also disclosed in U.S. Pat. No. 6,541,509, “Discodermolide also may provide a means to circumvent clinical resistance due to overproduction of P-glycoprotein. Accordingly, the combination of paclitaxel and discodermolide may be advantageous for use in subjects who exhibit resistance to paclitaxel (Taxol). Since Taxol is frequently utilized in the treatment of human cancers, a strategy to enhance its utility in the clinical setting, by combining its administration with that of discodermolide, may be of great benefit to many subjects suffering from malignant neoplasias, particularly advanced cancers.” The comments made regading discodermolide are equally applicable to applicants' magnetic anti-mitotic agent.

As is also disclosed in U.S. Pat. No. 6,541,509, “In the method of the present invention, administration of paclitaxel ‘in combination with’ discodermolide refers to co-administration of the two antineoplastic agents. Co-administration may occur concurrently, sequentially, or alternately. Concurrent co-administration refers to administration of both paclitaxel and discodermolide at essentially the same time. For concurrent co-administration, the courses of treatment with paclitaxel and with discodermolide may be run simultaneously. For example, a single, combined formulation, containing both an amount of paclitaxel and an amount of discodermolide in physical association with one another, may be administered to the subject. The single, combined formulation may consist of an oral formulation, containing amounts of both paclitaxel and discodermolide, which may be orally administered to the subject, or a liquid mixture, containing amounts of both paclitaxel and discodermolide, which may be injected into the subject.” The same means of administration may be used in the process of the instant inventin.

As is also disclosed in U.S. Pat. No. 6,541,509, “It is also within the confines of the present invention that an amount of paclitaxel and an amount of discodermolide may be administered concurrently to a subject, in separate, individual formulations. Accordingly, the method of the present invention is not limited to concurrent co-administration of paclitaxel and discodermolide in physical association with one another.” The same means of administration may be used in the process of the instant invention.

As is also disclosed in U.S. Pat. No. 6,541,509, “In the method of the present invention, paclitaxel and discodermolide also may be co-administered to a subject in separate, individual formulations that are spaced out over a period of time, so as to obtain the maximum efficacy of the combination. Administration of each drug may range in duration from a brief, rapid administration to a continuous perfusion. When spaced out over a period of time, co-administration of paclitaxel and discodermolide may be sequential or alternate. For sequential co-administration, one of the antineoplastic agents is separately administered, followed by the other. For example, a full course of treatment with paclitaxel may be completed, and then may be followed by a full course of treatment with discodermolide. Alternatively, for sequential co-administration, a full course of treatment with discodermolide may be completed, then followed by a full course of treatment with paclitaxel. For alternate co-administration, partial courses of treatment with paclitaxel may be alternated with partial courses of treatment with discodermolide, until a full treatment of each drug has been administered.” The same means of administration may be used in the process of the instant invention.

As is also disclosed in U.S. Pat. No. 6,541,509, “The antineoplastic agents of the present invention (i.e., paclitaxel and discodermolide, either in separate, individual formulations, or in a single, combined formulation) may be administered to a human or animal subject by known procedures, including, but not limited to, oral administration, parenteral administration (e.g., intramuscular, intraperitoneal, intravascular, intravenous, or subcutaneous administration), and transdermal administration. Preferably, the antineoplastic agents of the present invention are administered orally or intravenously.” The same means of administration may be used in the process of the instant invention.

As is also disclosed in U.S. Pat. No. 6,541,509, “For oral administration, the formulations of paclitaxel and discodermolide (whether individual or combined) may be presented as capsules, tablets, powders, granules, or as a suspension. The formulations may have conventional additives, such as lactose, mannitol, corn starch, or potato starch. The formulations also may be presented with binders, such as crystalline cellulose, cellulose derivatives, acacia, corn starch, or gelatins. Additionally, the formulations may be presented with disintegrators, such as corn starch, potato starch, or sodium carboxymethyl-cellulose. The formulations also may be presented with dibasic calcium phosphate anhydrous or sodium starch glycolate. Finally, the formulations may be presented with lubricants, such as talc or magnesium stearate.” The same means of administration may be used in the process of the instant invention.

As is also disclosed in U.S. Pat. No. 6,541,509, “For parenteral administration, the formulations of paclitaxel and discodermolide (whether individual or combined) may be combined with a sterile aqueous solution which is preferably isotonic with the blood of the subject. Such formulations may be prepared by dissolving a solid active ingredient in water containing physiologically-compatible substances, such as sodium chloride, glycine, and the like, and having a buffered pH compatible with physiological conditions, so as to produce an aqueous solution, then rendering said solution sterile. The formulations may be presented in unit or multi-dose containers, such as sealed ampules or vials. Moreover, the formulations may be delivered by any mode of injection, including, without limitation, epifascial, intracapsular, intracutaneous, intramuscular, intraorbital, intraperitoneal (particularly in the case of localized regional therapies), intraspinal, intrastemal, intravascular, intravenous, parenchymatous, or subcutaneous.” The same means of administration may be used in the process of the instant invention.

As is also disclosed in U.S. Pat. No. 6,541,509, “For transdermal administration, the formulations of paclitaxel and discodermolide (whether individual or combined) may be combined with skin penetration enhancers, such as propylene glycol, polyethylene glycol, isopropanol, ethanol, oleic acid, N-methylpyrrolidone, and the like, which increase the permeability of the skin to the antineoplastic agent, and permit the antineoplastic agent to penetrate through the skin and into the bloodstream. The antineoplastic agent/enhancer compositions also may be further combined with a polymeric substance, such as ethylcellulose, hydroxypropyl cellulose, ethylene/vinylacetate, polyvinyl pyrrolidone, and the like, to provide the composition in gel form, which may be dissolved in a solvent such as methylene chloride, evaporated to the desired viscosity, and then applied to backing material to provide a patch.” The same means of administration may be used in the process of the instant invention.

As is also disclosed in U.S. Pat. No. 6,541,509, “It is within the confines of the present invention that the formulations of paclitaxel and discodermolide (whether individual or combined) may be further associated with a pharmaceutically-acceptable carrier, thereby comprising a pharmaceutical composition. The pharmaceutically-acceptable carrier must be “acceptable” in the sense of being compatible with the other ingredients of the composition, and not deleterious to the recipient thereof. Examples of acceptable pharmaceutical carriers include Cremophor.™. (a common vehicle for Taxol), as well as carboxymethyl cellulose, crystalline cellulose, glycerin, gum arabic, lactose, magnesium stearate, methyl cellulose, powders, saline, sodium alginate, sucrose, starch, talc, and water, among others. Formulations of the pharmaceutical composition may conveniently be presented in unit dosage.” The same means of administration may be used in the process of the instant invention.

As is also disclosed in U.S. Pat. No. 6,541,509, “The formulations of the present invention may be prepared by methods well-known in the pharmaceutical art. For example, the active compound may be brought into association with a carrier or diluent, as a suspension or solution. Optionally, one or more accessory ingredients (e.g., buffers, flavoring agents, surface active agents, and the like) also may be added. The choice of carrier will depend upon the route of administration. The pharmaceutical composition would be useful for administering the antineoplastic agents of the present invention (i.e., paclitaxel and discodermolide, and their analogues and derivatives, either in separate, individual formulations, or in a single, combined formulation) to a subject to treat neoplasia. The antineoplastic agents are provided in amounts that are effective to treat neoplasia in the subject. These amounts may be readily determined by the skilled artisan.” Similar formulations may be used in the process of the instant invention.

As is also disclosed in U.S. Pat. No. 6,541,509, “It is also within the confines of the present invention that paclitaxel and discodermolide be co-administered in combination with radiation therapy or an antiangiogenic compound (either natural or synthetic). Examples of antiangiogenic compounds with which paclitaxel and discodermolide may be combined include, without limitation, angiostatin, tamoxifen, thalidomide, and thrombospondin.” Similar compositons may be used in the process of the instant invention.

As is also disclosed in U.S. Pat. No. 6,541,509, “The present invention further provides a synergistic combination of antineoplastic agents. As defined above, ‘antineoplastic’ refers to the ability to inhibit or prevent the development or spread of a neoplasm, and to limit, suspend, terminate, or otherwise control the maturation and proliferation of cells in a neoplasm. As used herein, a “synergistic combination of antineoplastic agents” refers to a combination of antineoplastic agents that achieves a greater antineoplastic effect than would otherwise result if the antineoplastic agents were administered individually. Additionally, as described above, the “antineoplastic agents” of the present invention are paclitaxel and discodermolide, and their analogues and derivatives, either in separate, individual formulations, or in a single, combined formulation. Administration of paclitaxel in combination with discodermolide unexpectedly results in a synergistic antineoplastic effect by providing greater efficacy than would result from use of either of the antineoplastic agents alone.” Similar synergistic combinations may be used in the process of the instant invention.

As is also disclosed in U.S. Pat. No. 6,541,509, “In the synergistic combination of the present invention, paclitaxel and discodermolide may be combined in a single formulation, such that the amount of paclitaxel is in physical association with the amount of discodermolide. This single, combined formulation may consist of an oral formulation, containing amounts of both paclitaxel and discodermolide, which may be orally administered to the subject, or a liquid mixture, containing amounts of both paclitaxel and discodermolide, which may be injected into the subject.” Similar synergistic combinations may be used in the process of the instant invention.

As is also disclosed in U.S. Pat. No. 6,541,509, “Alternatively, in the synergistic combination of the present invention, a separate, individual formulation of paclitaxel may be combined with a separate, individual formulation of discodermolide. For example, an amount of paclitaxel may be packaged in a vial or unit dose, and an amount of discodermolide may be packaged in a separate vial or unit dose. A synergistic combination of paclitaxel and discodermolide then may be produced by mixing the contents of the separate vials or unit doses in vitro. Additionally, a synergistic combination of paclitaxel and discodermolide may be produced in vivo by co-administering to a subject the contents of the separate vials or unit doses, according to the methods described above. Accordingly, the synergistic combination of the present invention is not limited to a combination in which amounts of paclitaxel and discodermolide are in physical association with one another in a single formulation.” Similar synergistic combinations may be used in the process of the instant invention.

As is also disclosed in U.S. Pat. No. 6,541,509, “The synergistic combination of the present invention comprises an effective antineoplastic amount of paclitaxel and an effective antineoplastic amount of discodermolide. As used herein, an ‘effective antineoplastic amount’ of paclitaxel or discodermolide is an amount of paclitaxel or discodermolide that is effective to ameliorate or minimize the clinical impairment or symptoms of neoplasia in a subject, in either a single or multiple dose. For example, the clinical impairment or symptoms of neoplasia may be ameliorated or minimized by diminishing any pain or discomfort suffered by the subject; by extending the survival of the subject beyond that which would otherwise be expected in the absence of such treatment; by inhibiting or preventing the development or spread of the neoplasm; or by limiting, suspending, terminating, or otherwise controlling the maturation and proliferation of cells in the neoplasm.” These comments are equally applicable to the process of the instant invention, in which discodermolide is replaced by the magnetic anti-mitotic compound of this invention.

As is also discussed in U.S. Pat. No. 6,541,509, “The effective antineoplastic amounts of paclitaxel and discodermolide will vary depending on the particular factors of each case, including the type of neoplasm, the stage of neoplasia, the subject's weight, the severity of the subject's condition, and the method of administration. For example, effective antineoplastic amounts of paclitaxel (Taxol) administered intraperitoneally may range from 1 to 10 mg/kg, and doses administered intravenously may range from 1 to 3 mg/kg, or from 135 mg/m2 to 200 mg/m2. Nevertheless, the appropriate effective antineoplastic amounts of paclitaxel and discodermolide can be readily determined by the skilled artisan.” These comments are equally applicable to the process of the instant invention, in which discodermolide is replaced by the magnetic anti-mitotic compound of this invention.

As is also disclosed in U.S. Pat. No. 6,541,509, “The synergistic combination described herein may be useful for treating neoplasia in a subject in need of treatment. Paclitaxel and discodermolide, which comprise the synergistic combination of the present invention, may be co-administered to a subject concurrently, sequentially, or alternately, as described above. Moreover, the paclitaxel and discodermolide of the present invention may be administered to a subject by any of the methods, and in any of the formulations, described above.” These comments are equally applicable to the process of the instant invention, in which discodermolide is replaced by the magnetic anti-mitotic compound of this invention.

By way of yet further illustration, and referring to published U.S. patent application 2003/0235855 (the entire disclosure of which is hereby incorporated by reference into this specification), claims an assay for the detection of paclitaxel resistant cells in human tumors. Claim 4 of this published patent application, which is typical, claims: “An isolated tubulin amino acid sequence comprising an amino acid sequence having at least one mutation, the mutation selected from the group consisting of a mutation at position 210, a mutation at position 214, a mutation at position 215, a mutation at position 216, a mutation at position 217, a mutation at position 225, a mutation at position 228, a mutation at position 270, a mutation at position 273, a mutation at position 292, and a mutation at position 365 and any combination thereof.”

At page 1 of published U.S. patent application 2003/0235855, the importance of paclitaxel is discussed. It is disclosed that “Paclitaxel (Taxol), Taxotere and other paclitaxel-like drugs that are currently under development hold great promise for the treatment of human cancer. Paclitaxel has shown remarkable activity against breast and ovarian cancer, melanomas, non-small lung carcinoma, esophogeal cancer, Kaposi's sarcoma, and some hematological malignancies. It has been described as the most significant antitumor drug developed in the last several decades and will, without doubt, find widespread use in the treatment of cancer. However, as is true of virtually all cancer chemotherapeutic drugs, patients responsive to paclitaxel eventually relapse due to the emergence of drug resistant tumor cells. Thus, there is a need in the art for methods to identify paclitaxel-resistant tumor cells, for agents that allow such identifications in a simple and cost effective way, and for methods for to treat patients with paclitaxel resistant tumor cells.” The solution presented to this problem in such published patent application is also described at page 1 thereof, wherein it is stated that: “The present invention involves polynucleotide mutations which confer paclitaxel resistance; mutant cells which are paclitaxel resistant; and methods to determine paclitaxel resistance. The present invention also provides a simple assay with sufficient sensitivity to detect drug resistant cells in tumor biopsies by extracting polynucleotide from the tissue. The extracted polynucleotide is then hybridized to mutant-specific PCR primers and the mutant regions of tubulin are identified by selective amplification. Once identified, a secondary treatment protocol can be administered to the patient to aid in tumor treatment.”

At pages 2 et seq. of published U.S. patent application 2003/00235855, the inventor discloses that “ . . . mutations able to conver resistance to paclitaxel are clustered in several small regions of beta-tubulin.” In paragraphs 0022 et seq., it is disclosed that: “The inventor has found that mutations able to confer resistance to paclitaxel are clustered in several small regions of β-tubulin (Tables I-III) including I210T, T214A, L215H, L215R, L215F, L215A, L215E, L215M, L215P, K216A, L217R, L217N, L217A, L225M, L228A, L228F, L228H, F270C, L273V, Q292H, and V365D. Of these 21 identified and sequenced mutant tubulins, 15 or 62% have a substitution at leucine including locations 215, 217, 225, 228 and 273. Of the 15 total leucine mutants, 7 or 46.7% occur at leu215, 3 or 20% occur at leu217, 3 or 20% occur at leu228, 1 or 6.7% occur at leu225 and 1 or 6.7% occur at leu273. The ability of 19 of the 21 total mutations to confer paclitaxel resistance has been confirmed by transfecting mutant cDNAs into wild-type cells.”

It is also disclosed in published U.S. patent application 2003/0235855 (commencing at page 3 thereof) that: “The clustering of mutations affecting leucines is unusual and unexpected. Also unexpected is the three relatively localized regions of mutation, 210-217, 225-228, and 270-273, and two isolated sites of mutations, 292 and 365. Although some of these regions appear distant in the primary structure, they are actually close together in the tertiary structure of β-tubulin. The data support the hypothesis that the mutations affect a critical interaction between tubulin subunits necessary for microtubule assembly and that the mechanism of paclitaxel is to facilitate this interaction.” Thereafter, in the middle of page 3 of such patent application, Table 1 is presented.

It is also disclosed in published U.S. patent application 2003/0235855 (commencing at page 3 thereof) that: “able V below contains the corresponding β-tubulin protein sequences for the variants listed in Table I: L215H (Seq. No. 10); L215R (Seq. No. 11); L215F (Seq. No. 12); L217R (Seq. No. 13); L228F (Seq. No. 14); and L228H (Seq. No. 15). All of these mutations result in amino acid substitutions at 3 leucine residues that are within 14 amino acids of one another.”

It is also disclosed in published U.S. patent application 2003/0235855 (commencing at page 3 thereof) that: “Using site-directed mutagenesis, the inventor has identified additional mutations in the H6/H7 loop of beta tubulin (that contains L215 and L217) that confer paclitaxel resistance. Table II lists the cell line, a portion of the encoding region including the mutated codon and the protein alteration.” Thereafer, Table II is presented on page 3 of the patent application.

It is also disclosed in published U.S. patent application 2003/0235855 (commencing at page 4 thereof) that: “The corresponding P-tubulin protein sequences (see Table IV) are: T214A (Seq. No. 24), L215A (Seq. No. 25), L215E (Seq. No. 26), L215M (Seq. No. 27), L215P (Seq. No. 28), K216A (Seq. No. 29), L217A (Seq. No. 30) and L228A (Seq. No. 31). The present invention also relates to probes having at least 12 bases including the codon for the particular amino acid substitution.”

It is also disclosed in published U.S. patent application 2003/0235855 (commencing at page 3 thereof) that: “More recently, the inventor has found that the number of mutations that confer resistance to paclitaxel are likely to be small and that most are clustered in a small region of β-tubulin. The likelihood that only a relatively small number of mutations will cause paclitaxel resistance is indicated by the observation that a random mutagenesis approach to find new mutations is recapitulating mutations that have already been found by classical genetics, and by the observation that mutations reported in different laboratories using different cell lines are beginning to show overlap. New mutants recently identified by the inventor in both CHO cells, and in the human KB3 cervical carcinoma cell line, are summarized in Table m. The fact that human mutations fall into the same region as the CHO mutations in the tertiary structure, combined with the observation that some mutations (not reported in this application) in CHO cells affect residues that are altered in human cell lines, supports the conclusion (based on identical amino acid sequences for β-tubulin in the two species) that mutations identified in CHO cells are expected to confer drug resistance in human cells. The nucleotide sequences encoding the new mutants are shown in Table III. 3 TABLE III “Thereafter, Table III is rpesented on page 4.

It is also disclosed in published U.S. patent application 2003/0235855 (commencing at page 4 thereof) that: “The new corresponding mutant CHO β-tubulin protein sequences (see Table IV) are: I210T (Ile to Thr at location 210) (Seq. No. 39), L217N (Leu to Asn at location 217) (Seq. No. 40), F270C (Phe to Cys at location 270) (Seq. No. 41) and Q292H (Gln to His at location 292) (Seq. No. 42). The new corresponding mutant human β-tubulin sequences are: L225M (Leu to Met at location 225) (Seq. No.43), L273V (Leu to Val at location 273) (Seq. No. 44) and V365D (Val to Asp at location 365) (Seq. No. 45).”

It is also disclosed in published U.S. patent application 2003/0235855 (commencing at page 4 thereof) that: “Table IV lists all of the nucleic acid and protein sequences in sequence order that are described in this application along with their sequence id number and abbreviated amino acid mutation.” Thereafter, Table IV is presented on pages 4 et seq.

It is also disclosed in United States published patent application 2003/0235855 (commencing at page 8 thereof) that: “Because α-tubulin and β-tubulin are similar proteins, similar clustering of mutations are anticipated in α-tubulin in paclitaxel resistant cells and α-tubulin PCR mutant primer sequences can be constructed in a similar manner to the primers presented herein for β-tubulin in paclitaxel resistant tumor cells.”

It is also disclosed in United States published patent application 2003/0235855 (commencing at page 8 thereof) that: “The assays of the present invention were performed using Chinese hamster ovary (CHO) cells selected for resistance to paclitaxel. It is important to note that human and hamster tubulin have identical amino acid sequences and the nucleotide sequences are highly homologous and the nucleotide differences do not alter the amino acid sequence, and therefore, the amino acid changes found in mutant CHO cells will also confer resistance in humans.”

It is also disclosed in United States published patent application 2003/0235855 (commencing at page 8 thereof) that: “It has been established that the most frequent mechanism of resistance to paclitaxel occurs through mutations in tubulin that affect the stability of the microtubules. These paclitaxel-resistant cells assemble less microtubule polymer and are frequently hypersensitive to other drugs such as vinblastine and vincristine that inhibit microtubule assembly.”

It is also disclosed in United States published patent application 2003/0235855 (commencing at page 8 thereof) that: “A model to explain these observations is provided in FIG. 1. The assay of the present invention can be used to identify many or most patients in danger of relapse due to tumor cell mutation and allow administration of alternate or additional treatment protocols using such agents as vinblastine or vincristine which are highly effective in eliminating the paclitaxel-resistant cells.”

It is also disclosed in United States published patent application 2003/0235855 (commencing at page 8 thereof) that: “The identification of the mutations and the clustering of mutations within the tubulin genes provide the data to construct highly efficient assays to detect these mutations in patients. Until now, there has been no method available to easily detect paclitaxel resistant cells in human tumors. The present methods or assays involve the design and use of allele-specific oligonucleotide primers for PCR.”

It is also disclosed in United States published patent application 2003/0235855 (commencing at page 8 thereof) that: “One such assay has been successfully confirmed for primers using the leu217 to arg mutation shown in FIG. 2. The wild-type primer (CTCCGTAGGTGGGCGTGGTGA (Seq. No.46)) is able to amplify wild-type DNA; but because of a 3′ mismatch with the mutant allele, it fails to amplify mutant DNA. Conversely, the mutant primer (CTCCGTAGGTGGGCGTGCGC (Seq. No. 47)) is able to amplify mutant DNA, but does not amplify the wild-type DNA because of 3′ mismatch (underlined). The mutant primer also contains an intentional mismatch to both wild-type and mutant DNA at the third nucleotide from the 3′ end (underlined) in order to enhance its allele specificity.”

It is also disclosed in United States published patent application 2003/0235855 (commencing at page 8 thereof) that: “Thus, allele-specific primers covering most potential mutations can be used individually or a ‘cocktails’ to detect the mutations in a single or very few PCR reactions. Alternatively, assays involving restriction enzyme digestion or allele-specific hybridization using the mutant DNA sequences can be used, but may lack the sensitivity and simplicity of the PCR assay.”

It is also disclosed in United States published patent application 2003/0235855 (commencing at page 9 thereof) that: “The high frequency of mutations affecting only a few leucine residues of α-tubulin in paclitaxel-resistant mutants was unexpected. Currently, there is no rational basis for predicting how an individual patient will respond to paclitaxel therapy. An initial assay of the tumor for mutations in tubulin that confer paclitaxel resistance would help clinicians decide whether the patient is a good candidate for paclitaxel therapy and save needless morbidity with a treatment that is unlikely to be effective. It would also allow the clinician to choose an alternative or additional therapy at an early time in the disease progression, thereby enhancing the survival of the patient.”

It is also disclosed in United States published patent application 2003/0235855 (commencing at page 9 thereof) that: “Mammals express 6 α- and 6 β-tubulin genes, which are the targeted genes. To further optimize assays, it may be necessary to determine which tubulin isotype is involved in paclitaxel resistance for each type of tumor in certain instances. The tubulin is expressed in a tissue specific manner, with some forms restricted to certain tissues, which are widely disclosed in the prior art literature. Furthermore, the present inventors have found in CHO cells that the most abundant tubulin isotype is the one always involved in conferring resistance, which was completely unexpected. Thus, one skilled in the art must merely find the most abundant isotype for each type of tumor, which is disclosed in many technical journal and prior art references.”

It is also disclosed in United States published patent application 2003/0235855 (commencing at page 9 thereof) that: “Paclitaxel is the prototype for a novel class of agents that inhibit cells in mitosis by promoting and stabilizing microtubule assembly. Early studies with this compound demonstrated that it binds to microtubules in a 1:1 stoichiometry with tubulin heterodimers (Manfredi, J. J., Parness, J., and Horwitz, S. B. (1981) J. Cell Biol. 94, 688-696) and inhibits microtubule disassembly. It is also able to induce microtubule assembly both in vitro and in vivo and induces microtubule bundle formation in treated cells (Schiff, P. B., Fant, J., and Horwitz, S. B. (1979) Nature 277, 665-667 and Schiff, P. B., and Horwitz, S. B. (1980) Proc. Natl. Acad. Sci. U.S.A. 77, 1561-1565). Recent interest in this and related compounds has been fueled by clinical studies demonstrating remarkable activity of paclitaxel against a number of malignant diseases (Rowinsky, E. K., and Donehower, R. C. (1995) N. E. J. Med. 332, 1004-1014). Although still in clinical trials, the demonstrated activity of paclitaxel in phase II studies has led to FDA approval for its use in refractory cases of breast and ovarian cancer. As more patients are treated with this drug, clinical resistance is expected to become an increasingly significant problem.”

It is also disclosed in United States published patent application 2003/0235855 (commencing at page 9 thereof) that: “The mechanisms by which tumor cells acquire resistance to paclitaxel are not fully understood. Cell culture studies have shown that paclitaxel is a substrate for the multidrug resistance pump (gP170), and cells selected for high levels of resistance to the drug have increased gP170 (Casazza, A. M., and Fairchild, C. R. (1996) Cancer Treatment & Research 87, 149-71). Nevertheless, it has yet to be demonstrated that this mechanism is significant in paclitaxel refractory tumors. Indeed, the remarkable efficacy of paclitaxel in early clinical studies of patients who were pretreated with Adriamycin, a well known substrate for gP170, argues that the multidrug resistance (mdr) phenotype may not be as clinically prevalent as had initially been anticipated (Schiff, P. B., and Horwitz, S. B. (1980) Proc. Natl. Acad. Sci. U.S.A. 77, 1561-1565).”

It is also disclosed in United States published patent application 2003/0235855 (commencing at page 9 thereof) that: “Additional mechanisms of resistance to paclitaxel have been reported. For example, several laboratories have provided evidence that changes in the expression of specific β-tubulin genes are associated with paclitaxel resistance in cultured tumor cell lines (Haber, M., Burkhart, C. A., Regl, D. L., Madafiglio, J., Norris, M. D., and Horwitz, S. B. (1995) J. Biol. Chem. 270, 31269-75; Jaffrezou, J. P., Dumontet, C., Derry, W. B., Duran, G., Chen, G., Tsuchiya, E., Wilson, L., Jordan, M. A., and Sikic, B. I. (1995) Oncology Res. 7, 517-27; Kavallaris, M., Kuo, D. Y. S., Burkhart, C. A., Regl, D. L., Norris, M. D., Haber, M., and Horwitz, S. B. (1997) J. Clin. Invest. 100, 1282-93; and Ranganathan, S., Dexter, D. W., Benetatos, C. A., and Hudes, G. R. (1998) Biochim. Biophys. Acta 1395, 237-245). More recently, a report describing mutations in P-tubulin that make the protein unresponsive to paclitaxel has appeared (Giannakakou, P., Sackett, D. L., Kang, Y.-K., Zhan, Z., Buters, J. T. M., Fojo, T., and Poruchynsky, M. S. (1997) J. Biol. Chem. 272, 17118-17125). To date, however, there is little evidence that any of the mechanisms described in cell culture cause paclitaxel resistance in human tumors.” It is also disclosed in United States published patent application 2003/0235855 (commencing at page 9 thereof) that “The inventor's own studies have described a resistance mechanism mediated by tubulin alterations that affect microtubule assembly (Cabral, F., and Barlow, S. B. (1991) Pharmac. Ther. 52, 159-171). Based on mutant properties and drug cross-resistance patterns, it is proposed that these changes in microtubule assembly could compensate for the presence of the drug (Cabral, F., Brady, R. C., and Schibler, M. J. (1986) Ann. N.Y. Acad. Sci. 466, 745-756). The inventors were later able to directly demonstrate that paclitaxel resistant Chinese hamster ovary (CHO) cells have diminished microtubule assembly compared to wild-type controls (Minotti, A. M., Barlow, S. B., and Cabral, F. (1991) J. Biol. Chem. 266,3987-3994). Thus, isolation of paclitaxel resistant mutants provides an opportunity to study mutations that not only give information about the mechanisms of drug action and resistance, but also give structural information about regions of tubulin that are involved in assembly.”

It is also disclosed in United States published patent application 2003/0235855 (commencing at page 10 thereof) that: “The inventors have now sequenced 9 mutant β-tubulin alleles and find that the mutations cluster at a site that is likely to be involved in lateral or longitudinal interactions during microtubule assembly. Remarkably, these mutations are present in the H6H7 region of of tubulin. Previously, it was believed that this region was not associated with paclitaxel binding. However, the inventors have isolated mutants in the H6H7 region, which are directly related to paclitaxel resistance.”

It is also disclosed in United States published patent application 2003/0235855 (commencing at page 10 thereof) that: “There is some significance to the fact that all the mutated residues are leucines—it certainly indicates that the changes that produce taxol resistance are not random. One possibility is that the leucines define a structural motif (e.g., analogous to a leucine zipper, but clearly distinct) that forms an interaction site with a neighboring subunit. A more trivial explanation is that the leucines are among the least critical residues in the region and are therefore better able to tolerate changes that produce the kind of subtle alterations in tubulin assembly that give resistance to taxol. The fact that the 3 leucines are highly conserved throughout all species and that the conservation extends to alpha and even gamma tubulin would tend to argue for the former alternative, but it will take a lot of further experimentation before the true significance can be elucidated.”

It is also disclosed in United States published patent application 2003/0235855 (commencing at page 10 thereof) that: “All 3 leucines in hamster are encoded by a CTC. Thus, a single base change can lead to substitution of histidine, arginine, phenylalanine, isoleucine, valine, or pro line. Only his, arg, and phe were isolated in the mutant cell lines. By transfection of cDNA altered by site-directed mutagenesis, is has been found that ile and val do not produce taxol resistance, probably because they do not perturb the structure of the microtubule sufficiently to produce resistance. Proline substitution can cause resistance, but appears to do so when expressed at very low levels. Moreover, the inventors have not been able to express it at high levels. This suggests that pro was not isolated in the mutant cell lines because it disrupts the structure of microtubules too severely for the cells to survive.”

It is also disclosed in United States published patent application 2003/0235855 (commencing at page 10 thereof) that: “The codons for leucine in human DNA are CTG at positions 215 and 217, and CTT at position 228. Single nucleotide changes will produce the same amino acid substitutions at 228, but a different set (valine, methionine, glutamine, arginine, or proline) at 215 and 217. Thus, 2 new possibilities (methionine and glutamine) might be found at 215 or 217 in human cells resistant to taxol. Of the two, methionine has been tested by transfection and it turns out to produce borderline resistance even at high levels of expression. A glutamine substitution has not yet been tested and should therefore be considered a presumptive candidate for producing resistance.”

A Preferred Anti-Mitotic Compound

In this section of the specification, a preferred compound is discussed. The preferred compound of this embodiment of the invention is an anti-mitotic compound. Anti-mitotic compounds are known to those skilled in the art. Reference may be had, e.g., to U.S. Pat. No. 6,723,858 (estrogenic compounds as anti-mitotic agents), U.S. Pat. No. 6,528,676 (estrogenic compounds as anti-mitotic agents), U.S. Pat. No. 6,350,777 (anti-mitotic agents which inhibit tubulin polyumerization), U.S. Pat. No. 6,162,930 (anti-mitotic agents which inhibit tubulin polymerization), U.S. Pat. No. 5,892,069 (estrogenic compounds as anti-mitotic agents), U.S. Pat. No. 5,886,025 (anti-mitotic agents which inhibit tubulin polymerization), U.S. Pat. No. 5,661,143 (estrogenic compounds as anti-mitotic agents), U.S. Pat. No. 3,997,506 (anti-mitotic derivatives of thiocolchicine), and the like. The entire disclosure of each of these United States patents applications is hereby incorporated by reference into this specification.

These prior art anti-mitotic agents may be modified, in accordance with the process of this invention, to make them “magnetic,” as that term is defined in this specification. In the next section of this specification, a process for modifying prior art taxanes to make them “magnetic” is described.

Preparation and Use of Magnetic Taxanes

In this portion of the specification, applicant will describe the preparation of certain magnetic taxanes that may be used in one or more of the processes of his invention. The process that is ued to make such taxanes magnetic and/or water soluble may also be used to make other anti-mitotic compounds magnetic and/or water soluble.

In one embodiment of the invention, a biologically active substrate is linked to a magnetic carrier particle. An external magnetic field may then be used to increase the concentration of a magnetically linked drug at a predetermined location.

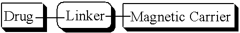

One method for the introduction of a magnetic carrier particle involves the linking of a drug with a magnetic carrier. While some naturally occurring drugs inherently carry magnetic particles (ferrimycin, albomycin, salmycin, etc.), it is more common to generate a synthetic analog of the target drug and attach the magnetic carrier through a linker.

Functionalized Taxanes

Paclitaxel and docetaxel are members of the taxane family of compounds. A variety of taxanes have been isolated from the bark and needles of various yew trees.

In one embodiment of the invention, such a linker is covalently attached to at least one of the positions in taxane.

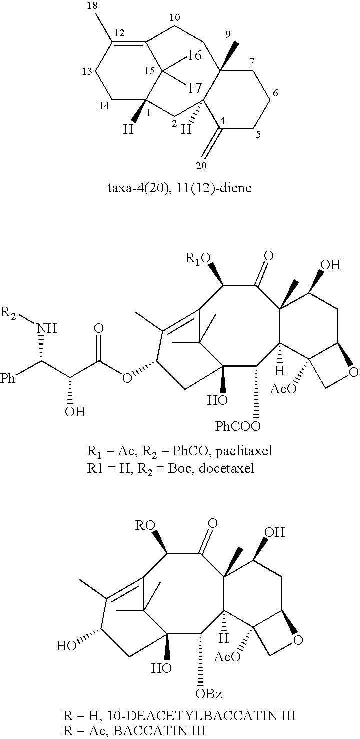

It is well known in the art that the northern hemisphere of taxanes has been altered without significant impact on the biological activity of the drug. Reference may be had to Chapter 15 of Taxane Anticancer Agents, Basic Science and Current Status, edited by G. George et al., ACS Symposium Series 583, 207^{th} National Meeting of the American Chemical Society, San Diego, Calif. (1994). Specifically the C-7, C-9, and C-10 positions of paclitaxel have been significantly altered without degrading the biological activity of the parent compound. Likewise the C-4 position appears to play only a minor role. The oxetane ring at C-4 to C-5 has been shown to be critical to biological activity. Likewise, certain functional groups on the C-13 sidechain have been shown to be of particular importance.

In one embodiment of the invention, a position within paclitaxel is functionalized to link a magnetic carrier particle. A number of suitable positions are presented below. It should be understood that paclitaxel is illustrated in the figures below, but other taxane analogs may also be employed. 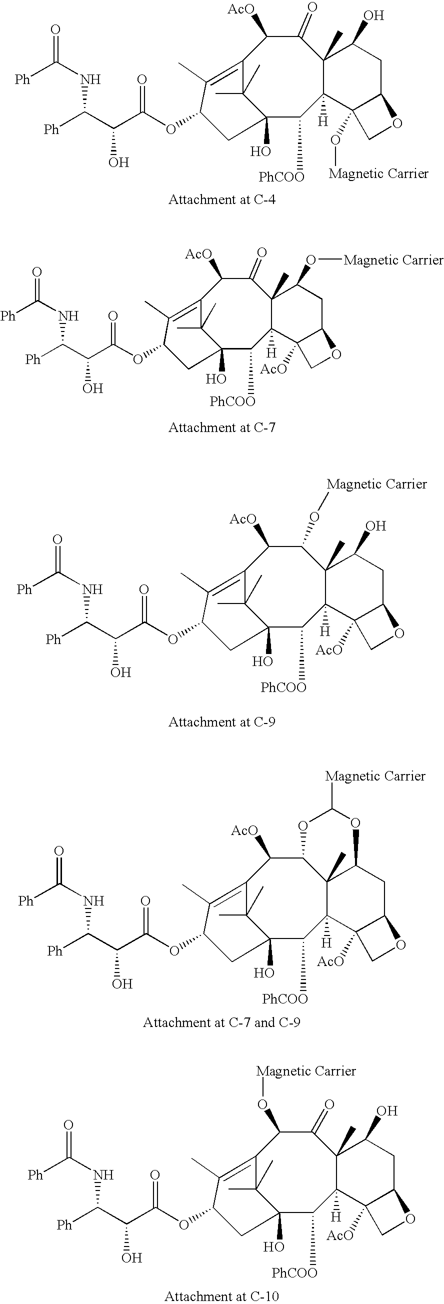 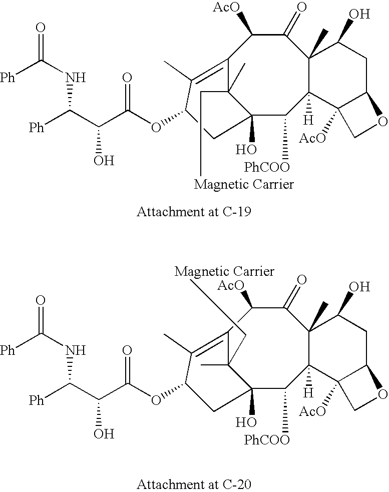
Attachment at C-4

C-4 taxane analogs have been previously generated in the art. A wide range of methodologies exist for the introduction of a variety of substituents at the C-4 position. By way of illustration, reference may be had to “Synthesis and Biological Evaluation of Novel C-4 Aziridine-Bearing Paclitaxel Analogs” by S. Chen et al., J. Med. Chem. 1995, vol 38, pp 2263.

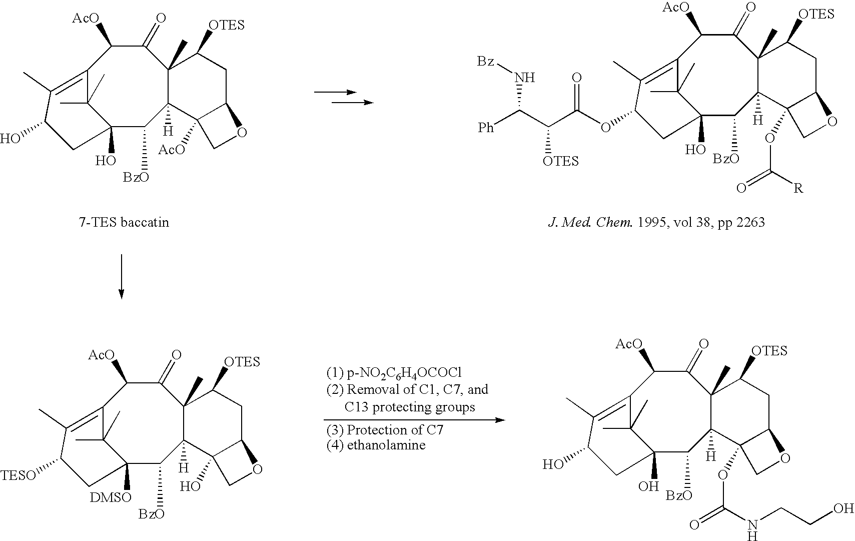

The secondary (C-13) and tertiary (C-1) alcohols of 7-TES baccatin were protected using the procedure of Chen (J. Org. Chem. 1994, vol 59, p 6156) while simultaneously unmasking the alcohol at C-4. The resulting product was treated with a chloroformate to yield the corresponding carboxylate. Removal of the silyl protecting groups at C-1, C-7, and C-13, followed by selective re-protection of the C-7 position gave the desired activated carboxylate. The compound was then treated with a suitable nucleophile (in the author's case, ethanolamine) to produce a C-4 functionalized taxane. The C-13 sidechain was installed using standard lactam methodology.

This synthetic scheme thus provides access to a variety of C-4 taxane analogs by simply altering the nucleophile used. In one embodiment of the instant invention, the nucleophile is selected so as to allow the attachment of a magnetic carrier to the C-4 position.

Attachment at C-7

The C-7 position is readily accessed by the procedures taught in U.S. Pat. No. 6,610,860. The alcohol at the C-10 position of 10-deacetylbaccatin III was selectively protected. The resulting product was then allowed to react with an acid halide to produce the corresponding ester by selectively acylating the C-7 position over the C-13 alcohol. Standard lactam methodology allowed the installation of the C-13 sidechain. In another embodiment, baccatin III, as opposed to its deacylated analog, is used as the starting material.

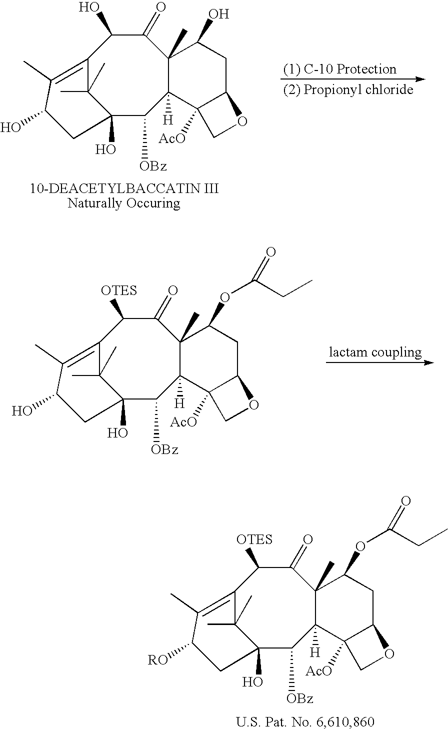

Other C-7 taxane analogs are disclosed in U.S. Pat. Nos. 6,610,860; 6,359,154; and 6,673,833, the contents of which are hereby incorporated by reference.

Attachment at C-9

It has been established that the C-9 carbonyl of paclitaxel is relatively chemically inaccessible, although there are exceptions (see, for example, Tetrahedron Lett. Vol 35, p 4999). However, scientists gained access to C-9 analogs when 13-acetyl-9-dihydrobaccatin III was isolated from Taxus candidensis (see J. Nat. Products, 1992, vol 55, p 55 and Tetrahedron Lett. 1992, vol 33, p 5173). This triol is currently used to provide access to a variety of such C-9 analogues.

In chapter 20 of Taxane Anticancer Agents, Basic Science and Current Status, (edited by G. George et al., ACS Symposium Series 583, 207^{th} National Meeting of the American Chemical Society, San Diego, Calif. (1994)) Klein describes a number of C-7/C-9 taxane analogs. One of routes discussed by Klein begins with the selective deacylation of 13-acetyl-9-dihydrobaccatin III, followed by the selective protection of the C7 alcohol as the silyl ether. A standard lactam coupling introduced the C-13 sidechain. The alcohols at C-7 and C-9 were sufficiently differentiated to allow a wide range of analogs to be generated. “In contrast to the sensitivity of the C-9 carbonyl series under basic conditions, the 9(R)-dihydro system can be treated directly with strong base in order to alkylate the C-7 and/or the C-9 hydroxyl groups.”

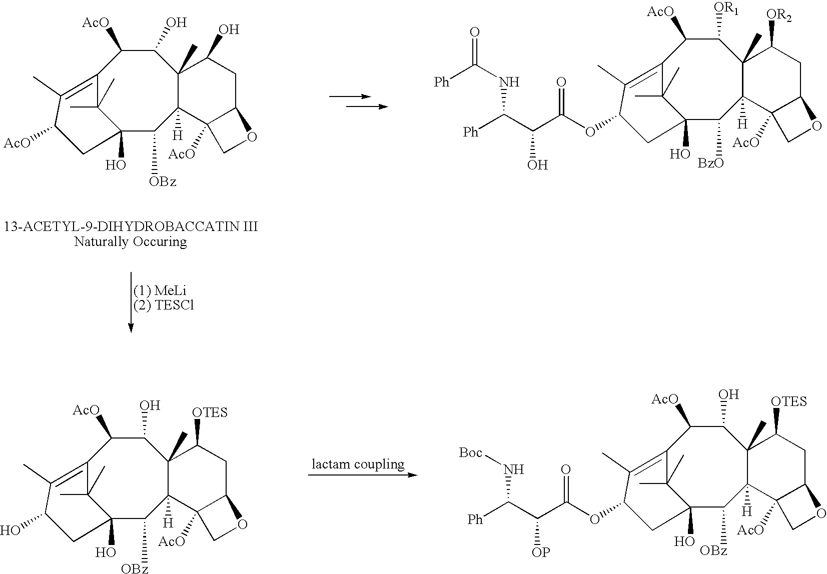

One skilled in the art may adapt Klein's general procedures to install a variety of magnetic carriers at these positions. Such minor adaptations are routine for those skilled in the art.

Attachment at C-7 and C-9

Klein also describes a procedure wherein 13-acetyl-9-dihydrobaccatin III is converted to 9-dihydrotaxol. Reference may be had to “Synthesis of 9-Dihydrotaxol: a Novel Bioactive Taxane” by L. L. Klein in Tetrahedron Lett. Vol 34, pp 2047-2050. An intermediate in this synthetic pathway is the dimethylketal of 9-dihydrotaxol.

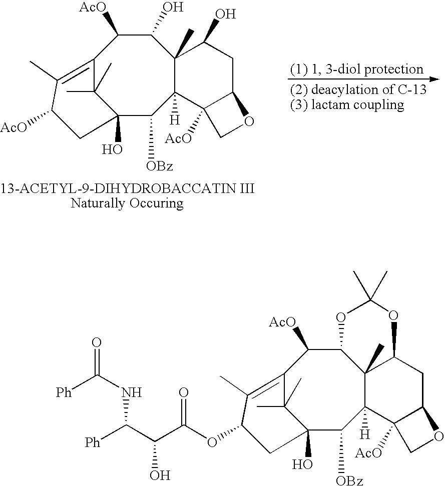

In one embodiment, the procedure of Klein is followed with a carbonyl compound other than acetone to bind a wide variety of groups to the subject ketal. Supplemental discussion of C-9 analogs is found in “Synthesis of 9-Deoxotaxane Analogs” by L. L. Klein in Tetrahedron Lett. Vol 35, p 4707 (1994).

Attachment at C-10

In one embodiment of the invention, the C-10 position is functionalized using the procedure disclosed in U.S. Pat. No. 6,638,973. This patent teaches the synthesis of paclitaxel analogs that vary at the C-10 position. A sample of 10-deacetylbaccatin III was acylated by treatment with propionic anhydride. The C-13 sidechain was attached using standard lactam methodology after first performing a selective protection of the secondary alcohol at the C-7 position. In one embodiment of the invention, this procedure is adapted to allow access to a variety of C-10 analogues of paclitaxel.

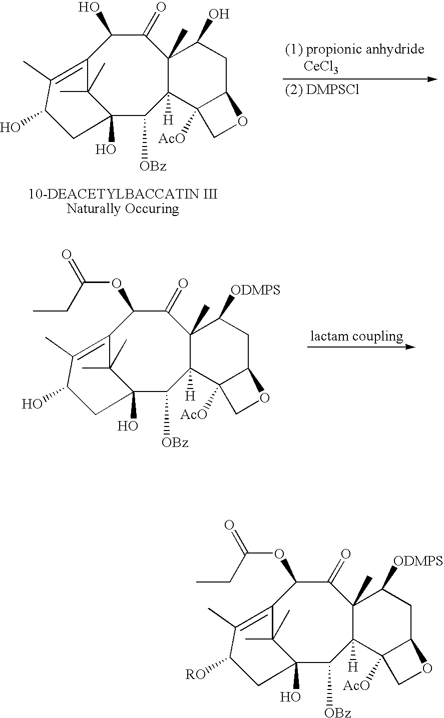

In one embodiment an anhydride is used as an electrophile. In another embodiment, an acid halide is used. As would be apparent to one of ordinary skill in the art, a variety of electrophiles could be employed. 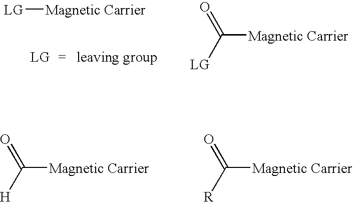
Siderophores

In one embodiment, a member of the taxane family of compounds is attached to a magnetic carrier particle. Suitable carrier particles include siderophores (both iron and non-iron containing), nitroxides, as well as other magnetic carriers.

Siderophores are a class of compounds that act as chelating agents for various metals. Most organisms use siderophores to chelate iron (III) although other metals may be exchanged for iron (see, for example, Exchange of Iron by Gallium in Siderophores by Emergy, Biochemistry 1986, vol 25, pages 4629-4633). Most of the siderophores known to date are either catecholates or hydroxamic acids.

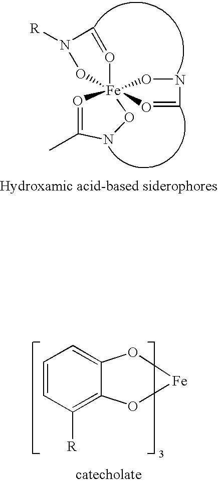

Representative examples of catecholate siderophores include the albomycins, agrobactin, parabactin, enterobactin, and the like.

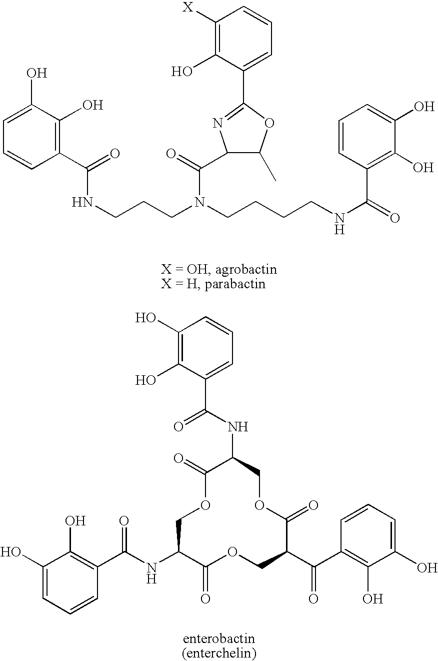

Examples of hydroxamic acid-based siderophores include ferrichrome, ferricrocin, the albomycins, ferrioxamines, rhodotorulic acid, and the like. Reference may be had to Microbial Iron Chelators as Drug Delivery Agents by M. J. Miller et al., Acc. Chem. Res. 1993, vol 26, pp 241-249; Structure of Des(diserylglycyl)ferrirhodin, DDF, a Novel Siderophore from Aspergillus ochraceous by M. A. F. Jalal et al., J. Org. Chem. 1985, vol 50, pp 5642-5645; Synthesis and Solution Structure of Microbial Siderophores by R. J. Bergeron, Chem. Rev. 1984, vol 84, pp 587-602; and Coordination Chemistry and Microbial Iron Transport by K. N. Raymond, Acc. Chem. Res., 1979, vol 12, pp 183-190. The synthesis of a retrohydroxamate analog of ferrichrome is described by R. K. Olsen et al. in J. Org. Chem. 1985, vol 50, pp 2264-2271.

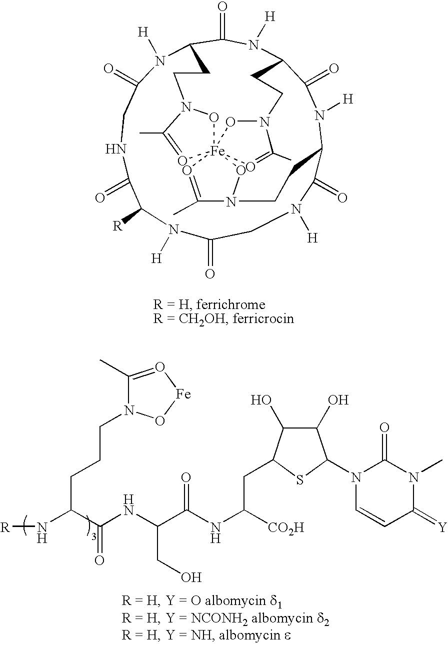

In “Total Synthesis of Desferrisalmycin” (M. J. Miller et al. in J. Am. Chem. Soc. 2002, vol 124 pp 15001-15005), a natural product is synthesized that contains a siderophore. The author states “siderophores are functionally defined as low molecular mass molecules which acquire iron (II) from the environment and transport it into microganisms. Because of the significant roles they play in the active transport of physiologically essentially iron (III) through microbe cell members, it is not surprising that siderophores-drug conjugates are attracting more and more attention from both medicinal chemists and clinical researchers as novel drug delivery systems in the war against microbial infections, especially in an area of widespread emergency of multidrug-resistance (MDR) strains. There have been three families of compounds identified as natural siderophore-drug conjugates, including ferrimycin, albomycin, and salmycin.” In a related paper, Miller describes the use of siderophores as drug delivery agents (Acc. Chem. Res. 1993, vol 26, pp 241-249. Presumably, the siderophore acts as a “sequestering agents [to] facilitate the active transport of chelated iron into cells where, by modification, reduction, or siderophore decomposition, it is released for use by the cell.” Miller describes the process of tethering a drug to a sidrophore to promote the active transport of the drug across the cell membrane.

In “The Preparation of a Fully Differentiated ‘Multiwarhead’ Sidrophore Precursor”, by M. J. Miller et al (J. Org. Chem. 2003, vol 68, pp 191-194) a precursor is disclosed which allows for a drug to be tethered to a sidrophore. In one embodiment, the route disclosed by Miller is employed to provide a variety of siderophores of similar structure. The synthesis of similar hydroxamic acid-based siderophores is discussed in J. Org. Chem. 2000, vol 65 (Total Synthesis of the Siderophore Danoxamine by M. J. Miller et al.), pp 4833-4838 and in the J. of Med. Chem. 1991, vol 32, pp 968-978 (by M. J. Miller et al.).

A variety of fluorescent labels have been attached to ferrichrome analogues in “Modular Fluorescent-Labeled Siderophore Analogues” by A. Shanzer et al. in J. Med. Chem. 1998, vol 41, 1671-1678. The authors have developed a general methodology for such attachments.

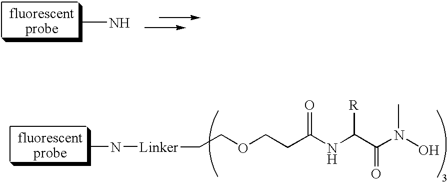

As discussed above, functionalized ferrichrome analogs have been previous generated, usually using basic amine acids (glycine). In one embodiment, functionality is introduced using an alternative amine acid (such as serine) in place of the central glycine residue. This provides a functional group foothold from which to base a wide variety of analogs. Using traditional synthetic techniques, various linkers are utilized so as to increase or decrease the distance between the magnetic carrier and the drug.

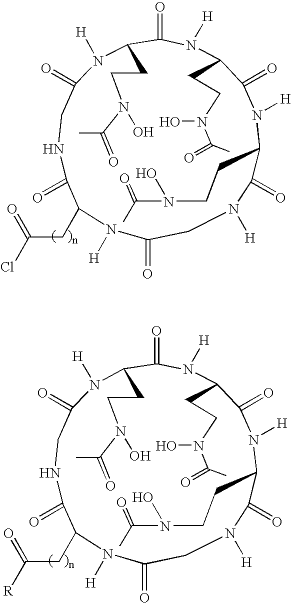

As would be apparent to one of ordinary skill in the art, the above specified techniques are widely applicable to a variety of substrates. By way of illustration, and not limitation, a number of magnetic taxanes are shown below. 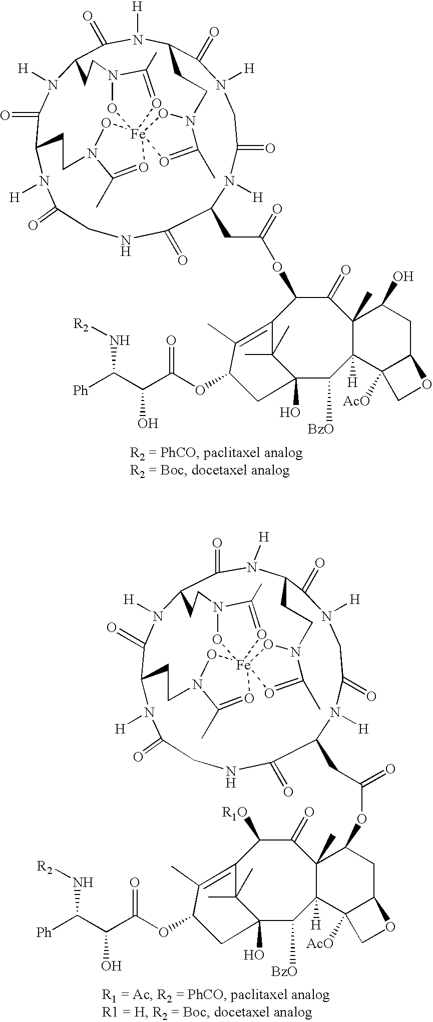 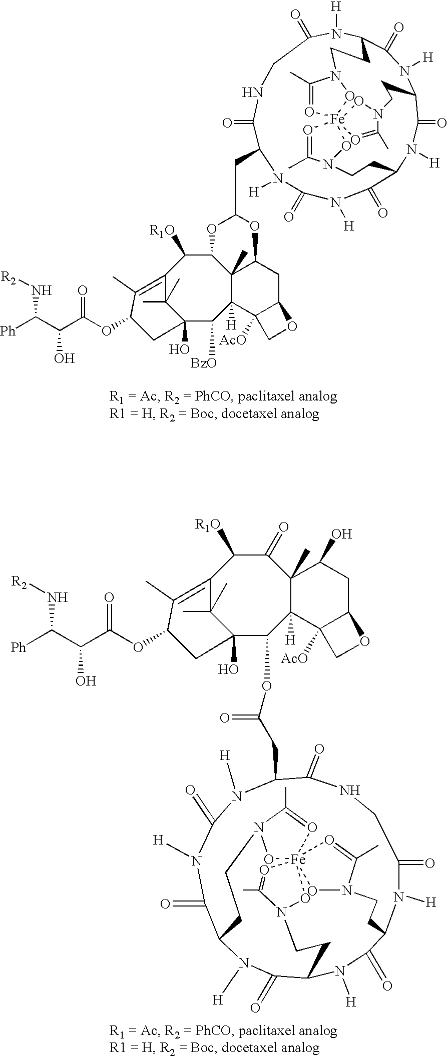
Nitroxides

Another class of magnetic carriers is the nitroxyl radicals (also known as nitroxides). Nitroxyl radicals a “persistent” radials that are unusually stable. A wide variety of nitroxyls are commercially available. Their paramagnetic nature allows them to be used as spin labels and spin probes.

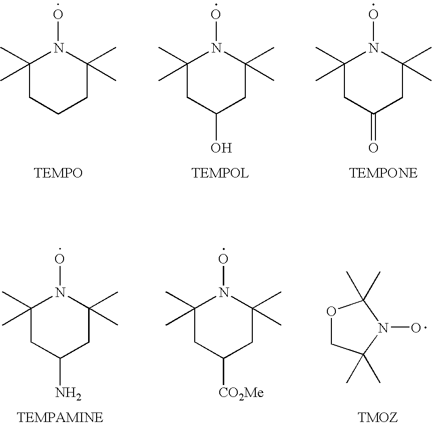

In addition to the commercially available nitroxyls, other paramagnetic radical labels have been generated by acid catalyzed condensation with 2-Amino-2-methyl-1-propanol followed by oxidation of the amine.

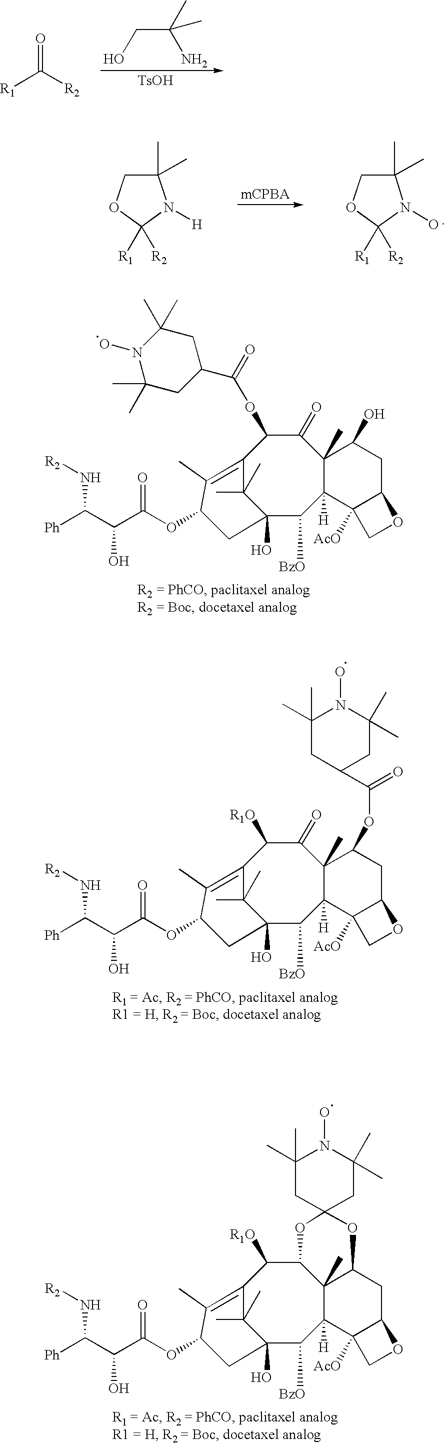

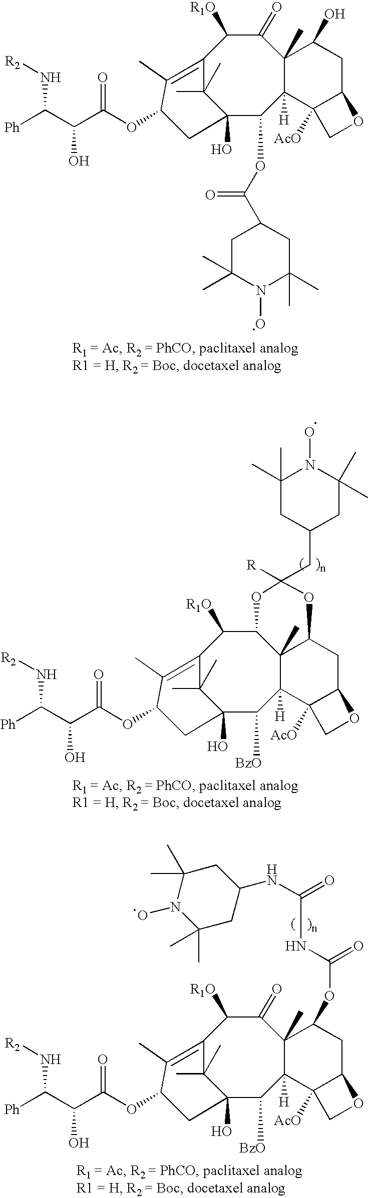

One of ordinary skill in the art could use the teachings of this specification to generate a wide variety of suitable carrier-drug complexes. The following table represents but a small sampling of such compounds.

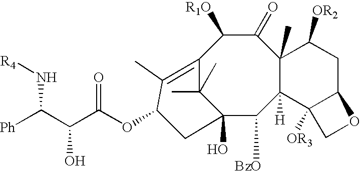

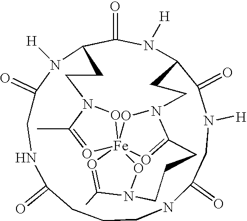

| R1 | R2 | R3 | R4 |
|---|---|---|---|
| F1, Y=CH2, | H | Ac | COPh |
| n=0 to 20 | | | |
| Ac | F1, Y=CH2, | Ac | COPh |
| | n=0 to 20 | | |
| Ac | H | F1, Y=CH2, | COPh |
| | | n=0 to 20 | |
| Ac | H | Ac | F1, Y=CH2, |
| | | | n=0 to 20 |
| H | H | Ac | Boc |
| F1, Y=CH2, | H | Ac | Boc |
| n=0 to 20 | | | |
| H | F1, Y=CH2, | Ac | Boc |
| | n=0 to 20 | | |
| H | H | F1, Y=CH2, | Boc |
| | | n=0 to 20 | |
| H | H | Ac | F1, Y=CH2, |
| | | | n=0 to 20 |
| F1, Y=NH or | H | Ac | COPh |
| NR, n=0 to 20 | | | |
| Ac | F1, Y=NH or | Ac | COPh |
| | NR, n=0 to 20 | | |
| Ac | H | F1, Y=NH or | COPh |
| | | NR, n=0 to 20 | |
| Ac | H | Ac | F1, Y=NH or |
| | | | NR, n=0 to 20 |
| H | H | Ac | Boc |
| F1, Y=NH or | H | Ac | Boc |
| NR, n=0 to 20 | | | |
| H | F1, Y=NH or | Ac | Boc |
| | NR, n=0 to 20 | | |
| H | H | F1, Y=NH or | Boc |
| | | NR, n=0 to 20 | |
| H | H | Ac | F1, Y=NH or |
| | | | NR, n=0 to 20 |
| N1, n=0 to 20 | H | Ac | COPh |
| Ac | N1, n=0 to 20 | Ac | COPh |
| Ac | H | N1, n=0 to 20 | COPh |
| Ac | H | Ac | N1, n=0 to 20 |
| H | H | Ac | Boc |
| N1, n=0 to 20 | H | Ac | Boc |
| H | N1, n=0 to 20 | Ac | Boc |
| H | H | N1, n=0 to 20 | Boc |
| H | H | Ac | N1, n=0 to 20 |
| N2, n=0 to 20, | H | Ac | COPh |
| X=O or NH | | | |
| Ac | N2, n=0 to 20, | Ac | COPh |
| | X=O or NH | | |
| Ac | H | N2, n=0 to 20, | COPh |
| | | X=O or NH | |
| Ac | H | Ac | N2, n=0 to 20, |
| | | | X=O or NH |
| H | H | Ac | Boc |
| N2, n=0 to 20, | H | Ac | Boc |
| X=O or NH | | | |
| H | N2, n=0 to 20, | Ac | Boc |
| | X=O or NH | | |
| H | H | N2, n=0 to 20, | Boc |
| | | X=O or NH | |
| H | H | Ac | N2, n=0 to 20, |
| | | | X=O or NH |
| N3, n=0 to 20, | H | Ac | COPh |
| X=O or NH | | | |
| Ac | N3, n=0 to 20, | Ac | COPh |
| | X=O or NH | | |
| Ac | H | N3, n=0 to 20, | COPh |
| | | X=O or NH | |
| Ac | H | Ac | N3, n=0 to 20, |
| | | | X=O or NH |
| H | H | Ac | Boc |
| N3, n=0 to 20, | H | Ac | Boc |
| X=O or NH | | | |
| H | N3, n=0 to 20, | Ac | Boc |
| | X=O or NH | | |
| H | H | N3, n=0 to 20, | Boc |
| | | X=O or NH | |
| H | H | Ac | N3, n=0 to 20, |
| | | | X=O or NH |
| F2 or F3 | H | Ac | COPh |
| Ac | F2 or F3 | Ac | COPh |
| Ac | H | F2 or F3 | COPh |
| Ac | H | Ac | F2 or F3 |
| F2 or F3 | H | Ac | Boc |
| H | F2 or F3 | Ac | Boc |
| H | H | F2 or F3 | Boc |
| H | H | Ac | F2 or F3 |

F2

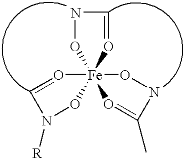

F3

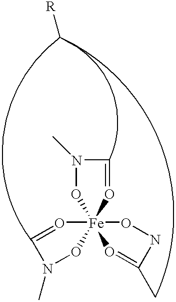

N1

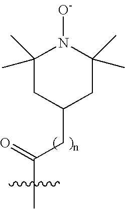

N2

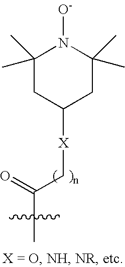

N3

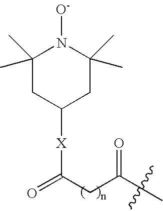

The prior disclosure illustrates how one may modify prior art taxanes to make them magnetic. As will be apparent to those skilled in the art, one may similarly modify other modifiable prior art anti-mitotic compounds to make them magnetic.

Other Modifiable Prior Art Compounds

Many anti-mitotic compounds that may be modified in accordance with the process of this invention are described in the prior art. One of these compounds is discodermolide; and it is described in U.S. Pat. No. 6,541,509, the entire disclosure of which is hereby incorporated by reference into this specification. Reference may be had, e.g., to column 10 of such paent and to the references 10, 11, 12, and 13 cited in such patent.

The reference 12 in U.S. Pat. No. 6,541,509 is to an article by R. J. Kowalski et al., “The Microtubule-Stabilizing Agent Discodermolide Competitively Inhibits the Binding of Paclitaxel (Taxol) to Tubulin Monomers, . . . ” Mol. Pharacol. 52:613-22, 1997. At page 2 of the Kowalski et al. patent, a formula for discodermolide is presented with 29 numbered carbon atoms (see FIG. 1).

Elsewhere in this specification, applicants teach how to make “magnetic taxanes” by incorporating therein various linker groups and/or siderophores. The same linker groups and/or siderphores may be utilized via subsgtantially the same process to make the discodermolide magnetic in the same manner.

As is disclosed elsewhere in this specification, siderphores are a class of compounds that act as chelating agents for various metals. When used to make “magnetic taxanes,” they are preferably bound to either the C7 and/or the C₁₀ carbons of the paclitaxels. They can similarly be used to make “magnetic discodermolides,” but in this latter case they should be bonded at the C17 carbon of discodermolide, to which a hydroxyl group is bound. The same linker that is used to link the C7/C10 carbon of the taxane to the siderphore may also be sued to link the C₁₋₇ carbon of the discodermolde to the siderphore.

In one embodiment, the “siderohophoric group” disclosed in U.S. Pat. No. 6,310,058, the entire disclosure of which is hereby incorporated by reference into this specification, is utilized. The siderophoric group is of the formula —(CH2)ₘ—N(OH)—C(O)—(CH₂)ₙ—(CH═CH)ₒ—CH3, wherein m is an integer of from 2 to 6, n is 0 or an integer of from 1 to 22, and o is 0 or an integer 1 to 4, provided that m+o is no greater than 25.

In another embodiment, “magentic epothilone A” and/or “magentic epotilone B” is also made by a similar process. As is also disclosed in the FIG. 1 of the Kowalski et al. article (see page 614), and in the formula depicted, the epothilone A exists when, in such formula, the alkyl group (“R”) is hydrogen, whereas the epothilone B exists when, in such formula, the alkyl group is methyl. In either case, one can make magnetic analogs of these compounds by using the same siderophores and the same linkers groups but utilzing them at a different site. One may bind such siderophores at either the number 3 carbon (which which a hydroxyl group is bound) and/or the number 7 carbon (to which another hydroxyl group is bound.).

Without wishing to be bound to any particular theory, applicants believe that the binding of the siderphores at the specified carbon sites imparts the required magnetic properties to such modified materials without adversely affecting the anti-mitotic properteis of the material. In fact, in some embodiment, the anti-mitotic properties of the modified magnetic materials surpass the anti-mitotic properties of the unmodified materials.

This is unexpected; for, if the same linker groups and/or siderophores are used to bind to other than the specified carbon atoms, materials with no or subtantially poorer anti-mitotic properties are produced.

Thus, e.g., and referring to the magnetic taxanes described elsewhere in this speficification (and also to FIG. 1 of the Kowalski et al. article), one should not link such siderphores to to any carbons on the pendant aromatic rings. Thus, e.g., and referring to the discodermolide structure, one should not link siderphores to any of 1, 2, 3, or 4 carbon atoms. Thus, e.g., and referring to the epothilones, one should not link the siderphores to any carbonon the ring structure containing sulfur and nitrogen.

By way of further illustration, and referring to U.S. Pat. Nos. 5,504,074, 5,661,143, 5,892,069, 6,528,676, and 6,723,858 (the entire disclosure of each of which is hereby incorporated by reference into this specification), one may modify estradiol and estradiol metabolites to make them magnetic in accordance with the process of this invention. As is disclosed in U.S. Pat. No. 6,723,858 (the entire disclosure of which is hereby incorporated by reference into this specification, “Cell mitosis is a multi-step process that includes cell division and replication (Alberts, B. et al. In The Cell, pp. 652-661 (1989); Stryer, E. Biochemistry (1988)). Mitosis is characterized by the intracellular movement and segregation of organelles, including mitotic spindles and chromosomes. Organelle movement and segregation are facilitated by the polymerization of the cell protein tubulin. Microtubules are formed from .alpha. and β tubulin polymerization and the hydrolysis of guanosine triphosphate (GTP). Microtubule formation is important for cell mitosis, cell locomotion, and the movement of highly specialized cell structures such as cilia and flagella.”

As is also disclosed in U.S. Pat. No. 6,723,858, “Microtubules are extremely labile structures that are sensitive to a variety of chemically unrelated anti-mitotic drugs. For example, colchicine and nocadazole are anti-mitotic drugs that bind tubulin and inhibit tubulin polymerization (Stryer, E. Biochemistry (1988)). When used Cell mitosis is a multi-step process that includes cell division and replication (Alberts, B. et al. In The Cell, pp. 652-661 (1989); Stryer, E. Biochemistry (1988)). Mitosis is characterized by the intracellular movement and segregation of organelles, including mitotic spindles and chromosomes. Organelle movement and segregation are facilitated by the polymerization of the cell protein tubulin. Microtubules are formed from .alpha. and β tubulin polymerization and the hydrolysis of guanosine triphosphate (GTP). Microtubule formation is important for cell mitosis, cell locomotion, and the movement of highly specialized cell structures such as cilia and flagella. Microtubules are extremely labile structures that are sensitive to a variety of chemically unrelated anti-mitotic drugs. For example, colchicine and nocadazole are anti-mitotic drugs that bind tubulin and inhibit tubulin polymerization (Stryer, E. Biochemistry (1988)). When used alone or in combination with other therapeutic drugs, colchicine may be used to treat cancer (WO-9303729-A, published Mar. 4, 1993; J 03240726-A, published Oct. 28, 1991), alter neuromuscular function, change blood pressure, increase sensitivity to compounds affecting sympathetic neuron function, depress respiration, and relieve gout (Physician's Desk Reference, Vol. 47, p. 1487, (1993)).”

As is also disclosed in U.S. Pat. No. 6,723,858, “Estradiol and estradiol metabolites such as 2-methoxyestradiol have been reported to inhibit cell division (Seegers, J. C. et al. J. Steroid Biochem. 32, 797-809 (1989); Lottering, M-L. et al. Cancer Res. 52, 5926-5923(1992); Spicer, L. J. and Hammond, J. M. Mol. and Cell. Endo. 64, 119-126 (1989); Rao, P. N. and Engelberg, J. Exp. Cell Res. 48, 71-81 (1967)). However, the activity is variable and depends on a number of in vitro conditions. For example, estradiol inhibits cell division and tubulin polymerization in some in vitro settings (Spicer, L. J. and Hammond, J. M. Mol. and Cell. Endo. 64, 119-126 (1989); Ravindra, R., J. Indian Sci. 64 (c) (1983)), but not in others (Lottering, M-L. et al. Cancer Res. 52, 5926-5923 (1992); Ravindra, R., J. Indian Sci. 64 (c) (1983)). Estradiol metabolites such as 2-methoxyestradiol will inhibit cell division in selected in vitro settings depending on whether the cell culture additive phenol red is present and to what extent cells have been exposed to estrogen. (Seegers, J. C. et al. Joint NCI-IST Symposium. Biology and Therapy of Breast Cancer. Sep. 25, Sep. 27, 1989, Genoa, Italy, Abstract A 58). alone or in combination with other therapeutic drugs, colchicine may be used to treat cancer (WO-9303729-A, published Mar. 4, 1993; J 03240726-A, published Oct. 28, 1991), alter neuromuscular function, change blood pressure, increase sensitivity to compounds affecting sympathetic neuron function, depress respiration, and relieve gout (Physician's Desk Reference, Vol. 47, p. 1487, (1993)).

As is also disclosed in U.S. Pat. No. 6,723,858, estradiol and estradiol metabolites such as 2-methoxyestradiol have been reported to inhibit cell division (Seegers, J. C. et al. J. Steroid Biochem. 32, 797-809 (1989); Lottering, M-L. et al. Cancer Res. 52, 5926-5923(1992); Spicer, L. J. and Hammond, J. M. Mol. and Cell. Endo. 64, 119-126 (1989); Rao, P. N. and Engelberg, J. Exp. Cell Res. 48, 71-81 (1967)). However, the activity is variable and depends on a number of in vitro conditions. For example, estradiol inhibits cell division and tubulin polymerization in some in vitro settings (Spicer, L. J. and Hammond, J. M. Mol. and Cell. Endo. 64, 119-126 (1989); Ravindra, R., J. Indian Sci. 64 (c) (1983)), but not in others (Lottering, M-L. et al. Cancer Res. 52, 5926-5923 (1992); Ravindra, R., J. Indian Sci. 64 (c) (1983)). Estradiol metabolites such as 2-methoxyestradiol will inhibit cell division in selected in vitro settings depending on whether the cell culture additive phenol red is present and to what extent cells have been exposed to estrogen. (Seegers, J. C. et al. Joint NCI-IST Symposium. Biology and Therapy of Breast Cancer. Sep. 25, Sep. 27, 1989, Genoa, Italy, Abstract A 58).

In one preferred embodiment, the modifiable anti-mitotic agent is an anti-microtubule agent. In one aspect of this embodiment, and referring to U.S. Pat. No. 6,689,803 at columns 5-6 thereof (the entire disclosure of which patent is hereby incorporated by reference into this specification), representative anti-microtubule agents include, e.g., “ . . . . taxanes (e.g., paclitaxel and docetaxel), campothecin, eleutherobin, sarcodictyins, epothilones A and B, discodermolide, deuterium oxide (D2 O), hexylene glycol (2-methyl-2,4-pentanediol), tubercidin (7-deazaadenosine), LY290181 (2-amino-4-(3-pyridyl)-4H-naphtho(1,2-b)pyran-3-cardonitrile), aluminum fluoride, ethylene glycol bis-(succinimidylsuccinate), glycine ethyl ester, nocodazole, cytochalasin B, colchicine, colcemid, podophyllotoxin, benomyl, oryzalin, majusculamide C, demecolcine, methyl-2-benzimidazolecarbamate (MBC), LY195448, subtilisin, 1069C85, steganacin, combretastatin, curacin, estradiol, 2-methoxyestradiol, flavanol, rotenone, griseofulvin, vinca alkaloids, including vinblastine and vincristine, maytansinoids and ansamitocins, rhizoxin, phomopsin A, ustiloxins, dolastatin 10, dolastatin 15, halichondrins and halistatins, spongistatins, cryptophycins, rhazinilam, betaine, taurine, isethionate, HO-221, adociasulfate-2, estramustine, monoclonal anti-idiotypic antibodies, microtubule assembly promoting protein (taxol-like protein, TALP), cell swelling induced by hypotonic (190 mosmol/L) conditions, insulin (100 nmol/L) or glutamine (10 mmol/L), dynein binding, gibberelin, XCHO1 (kinesin-like protein), lysophosphatidic acid, lithium ion, plant cell wall components (e.g., poly-L-lysine and extensin), glycerol buffers, Triton X-100 microtubule stabilizing buffer, microtubule associated proteins (e.g., MAP2, MAP4, tau, big tau, ensconsin, elongation factor-1-alpha (EF-1.alpha.) and E-MAP-115), cellular entities (e.g., histone H1, myelin basic protein and kinetochores), endogenous microtubular structures (e.g., axonemal structures, plugs and GTP caps), stable tubule only polypeptide (e.g., STOP145 and STOP220) and tension from mitotic forces, as well as any analogues and derivatives of any of the above. Within other embodiments, the anti-microtubule agent is formulated to further comprise a polymer.”

The term “anti-microtubule,” as used in this specification (and in the specification of U.S. Pat. No. 6,689,803), refers to any “ . . . protein, peptide, chemical, or other molecule which impairs the function of microtubules, for example, through the prevention or stabilization of polymerization. A wide variety of methods may be utilized to determine the anti-microtubule activity of a particular compound, including for example, assays described by Smith et al. (Cancer Lett 79(2):213-219, 1994) and Mooberry et al., (Cancer Lett. 96(2):261-266, 1995);” see, e.g., lines 13-21 of column 14 of U.S. Pat. No. 6,689,803. One preferred method, utilizing the anti-mitotic factor, is described in this specification.

An extensive listing of anti-microtubule agents is provided in columns 14, 15, 16, and 17 of U.S. Pat. No. 6,689,803; and one or more of them may be modified them in accordance with the process of this invention to make them magnetic. These anti-microtubule agents include “ . . . taxanes (e.g., paclitaxel (discussed in more detail below) and docetaxel) (Schiff et al., Nature 277: 665-667, 1979; Long and Fairchild, Cancer Research 54: 4355-4361, 1994; Ringel and Horwitz, J. Natl. Cancer Inst. 83(4): 288-291, 1991; Pazdur et al., Cancer Treat. Rev. 19(4): 351-386, 1993), campothecin, eleutherobin (e.g., U.S. Pat. No. 5,473,057), sarcodictyins (including sarcodictyin A), epothilones A and B (Bollag et al., Cancer Research 55: 2325-2333, 1995), discodermolide (ter Haar et al., Biochemistry 35: 243-250, 1996), deuterium oxide (D2 O) (James and Lefebvre, Genetics 130(2): 305-314, 1992; Sollott et al., J. Clin. Invest. 95: 1869-1876, 1995), hexylene glycol (2-methyl-2,4-pentanediol) (Oka et al., Cell Struct. Funct. 16(2): 125-134, 1991), tubercidin (7-deazaadenosine) (Mooberry et al., Cancer Lett. 96(2): 261-266, 1995), LY290181 (2-amino-4-(3-pyridyl)-4H-naphtho(1,2-b)pyran-3-cardonitrile) (Panda et al., J. Biol. Chem. 272(12): 7681-7687, 1997; Wood et al., Mol. Pharmacol. 52(3): 437-444, 1997), aluminum fluoride (Song et al., J. Cell. Sci. Suppl. 14: 147-150, 1991), ethylene glycol bis-(succinimidylsuccinate) (Caplow and Shanks, J. Biol. Chem. 265(15): 8935-8941, 1990), glycine ethyl ester (Mejillano et al., Biochemistry 31(13): 3478-3483, 1992), nocodazole (Ding et al., J. Exp. Med. 171(3): 715-727, 1990; Dotti et al., J. Cell Sci. Suppl. 15: 75-84, 1991; Oka et al., Cell Struct. Funct. 16(2): 125-134, 1991; Weimeret al., J. Cell. Biol. 136(1), 71-80, 1997), cytochalasin B (Illinger et al., Biol. Cell 73(2-3): 131-138, 1991), colchicine and CI 980 (Allen et al., Am. J. Physiol. 261(4 Pt. 1): L315-L321, 1991; Ding et al., J. Exp. Med. 171(3): 715-727, 1990; Gonzalez et al., Exp. Cell. Res. 192(1): 10-15, 1991; Stargell et al., Mol. Cell. Biol. 12(4): 1443-1450, 1992; Garcia et al., Antican. Drugs 6(4): 533-544, 1995), colcemid (Barlow et al., Cell. Motil. Cytoskeleton 19(1): 9-17, 1991; Meschini et al., J. Microsc. 176(Pt. 3): 204-210, 1994; Oka et al., Cell Struct. Funct. 16(2): 125-134, 1991), podophyllotoxin (Ding et al., J. Exp. Med. 171(3): 715-727, 1990), benomyl (Hardwick et al., J. Cell. Biol. 131(3): 709-720, 1995; Shero et al., Genes Dev. 5(4): 549-560, 1991), oryzalin (Stargell et al., Mol. Cell. Biol. 12(4): 1443-1450, 1992), majusculamide C (Moore, J. Ind. Microbiol. 16(2): 134-143, 1996), demecolcine (Van Dolah and Ramsdell, J. Cell. Physiol. 166(1): 49-56, 1996; Wiemer et al., J. Cell. Biol. 136(1): 71-80, 1997), methyl-2-benzimidazolecarbamate (MBC) (Brown et al., J. Cell. Biol. 123(2): 387-403, 1993), LY195448 (Barlow & Cabral, Cell Motil. Cytoskel. 19: 9-17, 1991), subtilisin (Saoudi et al., J. Cell Sci. 108: 357-367, 1995), 1069C85 (Raynaud et al., Cancer Chemother. Pharmacol. 35: 169-173, 1994), steganacin (Hamel, Med. Res. Rev. 16(2): 207-231, 1996), combretastatins (Hamel, Med. Res. Rev. 16(2): 207-231, 1996), curacins (Hamel, Med. Res. Rev. 16(2): 207-231, 1996), estradiol (Aizu-Yokata et al., Carcinogen. 15(9): 1875-1879, 1994), 2-methoxyestradiol (Hamel, Med. Res. Rev. 16(2): 207-231, 1996), flavanols (Hamel, Med. Res. Rev. 16(2): 207-231, 1996), rotenone (Hamel, Med. Res. Rev. 16(2): 207-231, 1996), griseofulvin (Hamel, Med. Res. Rev. 16(2): 207-231; 1996), vinca alkaloids, including vinblastine and vincristine (Ding et al., J. Exp. Med. 171(3): 715-727, 1990; Dirk et al., Neurochem. Res. 15(11): 1135-1139, 1990; Hamel, Med. Res. Rev. 16(2): 207-231, 1996; Illinger et al., Biol. Cell 73(2-3): 131-138, 1991; Wiemer et al., J. Cell. Biol. 136(1): 71-80, 1997), maytansinoids and ansamitocins (Hamel, Med. Res. Rev. 16(2): 207-231, 1996), rhizoxin (Hamel, Med. Res. Rev. 16(2): 207-231, 1996), phomopsin A (Hamel, Med. Res. Rev. 16(2): 207-231, 1996), ustiloxins (Hamel, Med. Res. Rev. 16(2): 207-231, 1996), dolastatin 10 (Hamel, Med Res. Rev. 16(2): 207-231, 1996), dolastatin 15 (Hamel, Med. Res. Rev. 16(2): 207-231, 1996), halichondrins and halistatins (Hamel, Med. Res. Rev. 16(2): 207-231, 1996), spongistatins (Hamel, Med. Res. Rev. 16(2): 207-231, 1996), cryptophycins (Hamel, Med. Res. Rev. 16(2): 207-231, 1996), rhazinilam (Hamel, Med. Res. Rev. 16(2): 207-231, 1996), betaine (Hashimoto et al., Zool. Sci. 1: 195-204, 1984), taurine (Hashimoto et al., Zool. Sci. 1: 195-204, 1984), isethionate (Hashimoto et al., Zool. Sci. 1: 195-204, 1984), HO-221 (Ando et al., Cancer Chemother. Pharmacol. 37: 63-69, 1995), adociasulfate-2 (Sakowicz et al., Science 280: 292-295, 1998), estramustine (Panda et al., Proc. Natl. Acad. Sci. USA 94: 10560-10564, 1997), monoclonal anti-idiotypic antibodies (Leu et al., Proc. Natl. Acad. Sci. USA 91(22): 10690-10694, 1994), microtubule assembly promoting protein (taxol-like protein, TALP) (Hwang et al., Biochem. Biophys. Res. Commun. 208(3): 1174-1180, 1995), cell swelling induced by hypotonic (190 mosmol/L) conditions, insulin (100 nmol/L) or glutamine (10 mmol/L) (Haussinger et al., Biochem. Cell. Biol. 72(1-2): 12-19, 1994), dynein binding (Ohba et al., Biochim. Biophys. Acta 1158(3): 323-332, 1993), gibberelin (Mita and Shibaoka, Protoplasma 119(1/2): 100-109, 1984), XCHO1 kinesin-like protein) (Yonetani et al., Mol. Biol. Cell 7(suppl): 211A, 1996), lysophosphatidic acid (Cook et al., Mol. Biol. Cell 6(suppl): 260A, 1995), lithium ion (Bhattacharyya and Wolff, Biochem. Biophys. Res. Commun. 73(2): 383-390, 1976), plant cell wall components (e.g., poly-L-lysine and extensin) (Akashi et al., Planta 182(3): 363-369, 1990), glycerol buffers (Schilstra et al., Biochem. J. 277(Pt. 3): 839-847, 1991; Farrell and Keates, Biochem. Cell. Biol. 68(11): 1256-1261, 1990; Lopez et al., J. Cell. Biochem. 43(3): 281-291, 1990), Triton X-100 microtubule stabilizing buffer (Brown et al., J. Cell Sci. 104(Pt. 2): 339-352, 1993; Safiejko-Mroczka and Bell, J. Histochem. Cytochem. 44(6): 641-656, 1996), microtubule associated proteins (e.g., MAP2, MAP4, tau, big tau, ensconsin, elongation factor-1-alpha EF-1.alpha.) and E-MAP-115) (Burgess et al., Cell Motil. Cytoskeleton 20(4): 289-300, 1991; Saoudi et al., J. Cell. Sci. 108(Pt. 1): 357-367, 1995; Bulinski and Bossler, J. Cell. Sci. 107(Pt. 10): 2839-2849, 1994; Ookata et al., J. Cell Biol. 128(5): 849-862, 1995; Boyne et al., J. Comp. Neurol. 358(2): 279-293, 1995; Ferreira and Caceres, J. Neurosci. 11(2): 392400, 1991; Thurston et al., Chromosoma 105(1): 20-30, 1996; Wang et al., Brain Res. Mol. Brain Res. 38(2): 200-208, 1996; Moore and Cyr, Mol. Biol. Cell 7(suppl): 221-A, 1996; Masson and Kreis, J. Cell Biol. 123(2), 357-371, 1993), cellular entities (e.g. histone H1, myelin basic protein and kinetochores) (Saoudi et al., J. Cell. Sci. 108(Pt. 1): 357-367, 1995; Simerly et al., J. Cell Biol. 111(4): 1491-1504, 1990), endogenous microtubular structures (e.g., axonemal structures, plugs and GTP caps) (Dye et al., Cell Motil. Cytoskeleton 21(3): 171-186, 1992; Azhar and Murphy, Cell Motil. Cytoskeleton 15(3): 156-161, 1990; Walker et al., J. Cell Biol. 114(1): 73-81, 1991; Drechsel and Kirschner, Curr. Biol. 4(12): 1053-1061, 1994), stable tubule only polypeptide (e.g., STOP 145 and STOP220) (Pirollet et al., Biochim. Biophys. Acta 1160(1): 113-119, 1992; Pirollet et al., Biochemistry 31(37): 8849-8855, 1992; Bosc et al., Proc. Natl. Acad. Sci. USA 93(5): 2125-2130, 1996; Margolis et al., EMBO J. 9(12): 4095-4102, 1990) and tension from mitotic forces (Nicklas and Ward, J. Cell Biol. 126(5): 1241-1253, 1994), as well as any analogues and derivatives of any of the above. Such compounds can act by either depolymerizing microtubules (e.g., colchicine and vinblastine), or by stabilizing microtubule formation (e.g., paclitaxel).”

U.S. Pat. No. 6,689,803 also discloses (at columns 16 and 17 that, “Within one preferred embodiment of the invention, the anti-mitotic compound is paclitaxel, a compound which disrupts microtubule formation by binding to tubulin to form abnormal mitotic spindles. Briefly, paclitaxel is a highly derivatized diterpenoid (Wani et al., J. Am. Chem. Soc. 93:2325, 1971) which has been obtained from the harvested and dried bark of Taxus brevifolia (Pacific Yew) and Taxomyces Andreanae and Endophytic Fungus of the Pacific Yew (Stierle et al., Science 60:214-216,-1993). “Paclitaxel” (which should be understood herein to include prodrugs, analogues and derivatives such as, for example, TAXOL®, TAXOTERE®, Docetaxel, 10-desacetyl analogues of paclitaxel and 3′N-desbenzoyl-3′N-t-butoxy carbonyl analogues of paclitaxel) may be readily prepared utilizing techniques known to those skilled in the art (see e.g., Schiff et al., Nature 277:665-667, 1979; Long and Fairchild, Cancer Research 54:4355-4361, 1994; Ringel and Horwitz, J. Natl. Cancer Inst. 83(4):288-291, 1991; Pazdur et al., Cancer Treat. Rev. 19(4):351-386, 1993; WO 94/07882; WO 94/07881; WO 94/07880; WO 94/07876; WO 93/23555; WO 93/10076; WO94/00156; WO 93/24476; EP 590267; WO 94/20089; U.S. Pat. Nos. 5,294,637; 5,283,253; 5,279,949; 5,274,137; 5,202,448; 5,200,534; 5,229,529; 5,254,580; 5,412,092; 5,395,850; 5,380,751; 5,350,866; 4,857,653; 5,272,171; 5,411,984; 5,248,796; 5,248,796; 5,422,364; 5,300,638; 5,294,637; 5,362,831; 5,440,056; 4,814,470; 5,278,324; 5,352,805; 5,411,984; 5,059,699; 4,942,184; Tetrahedron Letters 35(52):9709-9712, 1994; J. Med. Chem. 35:4230-4237, 1992; J. Med. Chem. 34:992-998, 1991; J. Natural Prod. 57(10):1404-1410, 1994; J. Natural Prod. 57(11):1580-1583, 1994; J. Am. Chem. Soc. 110:6558-6560, 1988), or obtained from a variety of commercial sources, including for example, Sigma Chemical Co., St. Louis, Mo. (T7402—from Taxus brevifolia).”

As is also disclosed in U.S. Pat. No. 6,689,893, “Representative examples of such paclitaxel derivatives or analogues include 7-deoxy-docetaxol, 7,8-cyclopropataxanes, N-substituted 2-azetidones, 6,7-epoxy paclitaxels, 6,7-modified paclitaxels, 10-desacetoxytaxol, 10-deacetyltaxol (from 10-deacetylbaccatin III), phosphonooxy and carbonate derivatives of taxol, taxol 2′,7-di(sodium 1,2-benzenedicarboxylate, 10-desacetoxy-11,12-dihydrotaxol-10,12(18)-diene derivatives, 10-desacetoxytaxol, Protaxol(2′- and/or 7-O-ester derivatives), (2′- and/or 7-O-carbonate derivatives), asymmetric synthesis of taxol side chain, fluoro taxols, 9-deoxotaxane, (13-acetyl-9-deoxobaccatine III, 9-deoxotaxol, 7-deoxy-9-deoxotaxol, 10-desacetoxy-7-deoxy-9-deoxotaxol, Derivatives containing hydrogen or acetyl group and a hydroxy and tert-butoxycarbonylamino, sulfonated 2′-acryloyltaxol and sulfonated 2′-O-acyl acid taxol derivatives, succinyltaxol, 2′-.gamma.-aminobutyryltaxol formate, 2′-acetyl taxol, 7-acetyl taxol, 7-glycine carbamate taxol, 2′-OH-7-PEG(5000)carbamate taxol, 2′-benzoyl and 2′,7-dibenzoyl taxol derivatives, other prodrugs (2′-acetyl taxol; 2′,7-diacetyltaxol; 2′succinyltaxol; 2′-(beta-alanyl)-taxol); 2′gamma-aminobutyryltaxol formate; ethylene glycol derivatives of 2′-succinyltaxol; 2′-glutaryltaxol; 2′-(N,N-dimethylglycyl)taxol; 2′-(2-(N,N-dimethylamino)propionyl)taxol; 2′orthocarboxybenzoyl taxol; 2′aliphatic carboxylic acid derivatives of taxol, Prodrugs {2′(N,N-diethylaminopropionyl)taxol, 2′(N,N-dimethylglycyl)taxol, 7(N,N-dimethylglycyl)taxol, 2′,7-di-(N,N-dimethylglycyl)taxol, 7(N,N-diethylaminopropionyl)taxol, 2′,7-di(N,N-diethylaminopropionyl)taxol, 2′-(L-glycyl)taxol, 7-(L-glycyl)taxol, 2′,7-di(L-glycyl)taxol, 2′-(L-alanyl)taxol, 7-(L-alanyl)taxol, 2′,7-di(L-alanyl)taxol, 2′-(L-leucyl)taxol, 7-(L-leucyl)taxol, 2′,7-di(L-leucyl)taxol, 2′-(L-isoleucyl)taxol, 7-(L-isoleucyl)taxol, 2′,7-di(L-isoleucyl)taxol, 2′-(L-valyl)taxol, 7-(L-valyl)taxol, 2′,7-di(L-valyl)taxol, 2′-(L-phenylalanyl)taxol, 7-(L-phenylalanyl)taxol, 2′,7-di(L-phenylalanyl)taxol, 2′-(L-prolyl)taxol, 7-(L-prolyl)taxol, 2′,7-di(L-prolyl)taxol, 2′-(L-lysyl)taxol, 7-(L-lysyl)taxol, 2′,7-di(L-lysyl)taxol, 2′-(L-glutamyl)taxol, 7-(L-glutamyl)taxol, 2′,7-di(L-glutamyl)taxol, 2′-(L-arginyl)taxol, 7-(L-arginyl)taxol, 2′,7-di(L-arginyl)taxol}, Taxol analogs with modified phenylisoserine side chains, taxotere, (N-debenzoyl-N-tert-(butoxycaronyl)-10-deacetyltaxol, and taxanes (e.g., baccatin III, cephalomannine, 10-deacetylbaccatin III, brevifoliol, yunantaxusin and taxusin).”

By way of yet further illustration, one may use one or more of the anti-mitotic agents disclosed in U.S. Pat. No. 6,673,937 (syntheses and methods of use of new antimitotic agents), U.S. Pat. No. 6,624,317 (taxoid conjugates as antimitotoic and antitumor agents), U.S. Pat. No. 6,593,334 (camptothecin-taxoid conjugates as antimitotic and antitumor agents), U.S. Pat. No. 6,593,321 (2-alkoxyestradiiol analogs with antiproliferative and antimitotic activity), U.S. Pat. No. 6,569,870 (fluorinated quinolones as antimitotic and antitumor agent), U.S. Pat. No. 6,528,489 (mycotoxin derivatives as antimitotic agents), U.S. Pat. No. 6,392,055 (synthesis and biological evaluation of analogs of the antimitotic marine natural product curacin A), U.S. Pat. No. 6,127,377 (vinka alkaloid antimitotic halogenated derivatives), U.S. Pat. No. 5,695,950 (method of screening for antimitotic compounds using the cdc25 tyrosine phosphatase), U.S. Pat. No. 5,620,985 (antimitotic binary alkaloid derivatives from catharanthus roseus), U.S. Pat. No. 5,294,538 (method of screening for antimitotic compounds using the CDC tyrosine phosphatase), and the like. The entire disclosure of each of these United States patents is hereby incorporated by reference into this specification.

As will be apparent, one or more of the aforementioned anti-mitotic and/or anti-microtubule agents may be modified to make them magnetic in accordance with this invention.

Synergistic Combinations of Magnetic Anti-Mitotic Agents

In one embodiment of this invention, discussed elsewhere in this specification, a synergistic combination of the magnetic anti-mititoic compound of this invention and paclitaxel is described. In the embodiment of the invention described in this section of the specification, a synergitic combination of two or more anti-mititoic compounds is described.

In one embodiment, the first anti-mitotic compound is preferably a magentic taxane such as, e.g., magentic paclitaxel and/or magnetic docetaxel. In this embodiment, the second anti-mitotic compound may be magnetic discdermolide, and/or magnetic epothilone A, and/or magentic epothilone B, and/or mixtures thereof. Other suitable combinations of magnetic anti-mitotic agents will be apparent.

Properties of the Preferred Anti-Mitotic Compounds

In one preferred embodiment, the compound of this invention has a mitotic index factor of at least about 10 percent and, more preferably, at least about 20 percent. In one aspect of this embodiment, the mitotic index factor is at least about 30 percent. In another embodiment, the mitotic index factor is at least about 50 percent.

In another embodiment of the invention, the compound of this invention has a mitotic index factor of less than about 5 percent.

As is known to those skilled in the art, the mitotic index is a measure of the extent of mitosis. Reference may be had, e.g., to U.S. Pat. No. 5,262,409 (binary tumor therapy), U.S. Pat. No. 5,443,962 (methods of indentifying inhibitors of cdc25 phosphatase), U.S. Pat. No. 5,744,300 (methods and reagents for the indentificatioin and regulation of senescence-related genes), U.S. Pat. Nos. 6,613,318, 6,251,585 (assay and reagents for indentifying anti-proliferative agents), U.S. Pat. No. 6,252,058 (sequences for targeting metastatic cells), U.S. Pat. No. 6,387,642 (method for indentifying a reagent that modulates Mytl activity), U.S. Pat. No. 6,413,735 (method of screening for a modulator of angiogenesis), U.S. Pat. No. 6,531,479 (anti-cancer compounds), U.S. Pat. No. 6,599,694 (method of characterizing potential therapeutics by determining cell-cell interactions), U.S. Pat. No. 6,620,403 (in vivo chemosensitivity screen for human tumors), U.S. Pat. No. 6,699,854 (anti-cancer compounds), U.S. Pat. No. 6,743,576 (database system for predictive cellular bioinformatics), and the like. The entire disclosure of each of these United States patents is hereby incorporated by reference into this specification.

Reference may also be had, e.g., to U.S. Pat. No. 5,262,409, which discloses that: “Determination of mitotic index: For testing mitotic blockage with nocodazole and taxol, cells were grown a minimum of 16 hours on polylysinecoated glass coverslips before drug treatment. Cells were fixed at intervals, stained with antibodies to detect lamin B, and counterstained with propidium iodide to assay chromosome condensation. To test cell cycle blocks in interphase, cells were synchronized in mitosis by addition of nocodazole (Sigma Chemical Co.) to a final concentration of 0.05 μg/ml from a 1 mg/ml stock in dimethylsulfoxide. After 12 hours arrest, the mitotic subpopulation was isolated by shakeoff from the culture plate. After applying cell cycle blocking drugs and/or 2-AP, cells were fixed at intervals, prepared for indirect immunofluorescence with anti-tubulin antibodies, and counterstained with propidium iodide. All data timepoints represent averages of three counts of greater than 150 cells each. Standard deviation was never more than 1.5% on the ordinate scale.”

Reference may be had, e.g., to U.S. Pat. No. 6,413,735 which discloses that: “The mitotic index is determined according to procedures standard in the art. Keram et al., Cancer Genet. Cytogenet. 55:235 (1991). Harvested cells are fixed in methanol:acetic acid (3:1, v:v), counted, and resuspended at 106 cells/ml in fixative. Ten microliters of this suspension is placed on a slide, dried, and treated with Giemsa stain. The cells in metaphase are counted under a light microscope, and the mitotic index is calculated by dividing the number of metaphase cells by the total number of cells on the slide. Statistical analysis of comparisons of mitotic indices is performed using the 2-sided paired t-test.”

By means of yet further illustration, one may measure the mitotic index by means of the procedures described in, e.g., articles by Keila Torres et al. (“Mechanisms of Taxol-Induced Cell Death are Concentration Dependent,” Cancer Research 58, 3620-3626, Aug. 15, 1998), and Jie-Gung Chen et al. (“Differential Mitosis Responses to Microtubule-stabilizing and destablilizng Drugs,” Cancer Research 62, 1935-1938, Apr. 1, 2002).

The mitotic index is preferably measured by using the well-known HeLa cell lines. As is known to those skilled in the art, HeLa cells are cells that have been derived from a human carcinoma of the cervix from a patient named Henrietta Lack; the cells have been maintained in tissued culture since 1953.

Hela cells are described, e.g., in U.S. Pat. No. 5,811,282 (cell lines useful for detection of human immunodeficiency virus), U.S. Pat. No. 5,376,525 (method for the detectioin of mycoplasma), U.S. Pat. Nos. 6,143,512, 6,326,196, 6,365,394 (cell lines and constructs useful in production of E-1 deleted adenoviruses), U.S. Pat. No. 6,440,658 (assay method for determining effect on aenovirus infection of Hela cells), U.S. Pat. No. 6,461,809 (method of improving inflectivity of cells for viruses), U.S. Pat. Nos. 6,596,535, 6,605,426, 6,610,493 (screening compounds for the ability to alter the production of amyloid-beta-peptide), U.S. Pat. No. 6,699,851 (cytotoxic compounds and their use), and the like; the entire disclosure of each of these United States patents is hereby incorporated by reference into this specification. By way of illustration, U.S. Pat. No. 6,440,658 This patent discloses that, for the experiments described in such patent, “The HeLa cell line was obtained from the American Type Culture Collection, Manassas Va.”

In one preferred embodiment, the mitotic index of a “control cell line” (i.e., one that omits that drug to be tested) and of a cell line that includes 50 nanomoles of such drug per liter of the cell line are determined and compared. The “mitotic index factor” is equal to (Mt−Mc/Mc)×100, wherein Mc is the mitotic index of the “control cell line,” and Mt is the mitotic index of the cell line that includes the drug to be tested.

The compound of this invention preferably has a molecular weight of at least about 150 grams per mole. In one embodiment, the molecular weight of such compound is at least 300 grams per mole. In another embodiment, the molecular weight of such compound is 400 grams per mole. In yet another embodiment, the molecular weight of such compound is at least about 550 grams per mole. In yet another embodiment, the molecular weight of such compound is at least about 1,000 grams per mole. In yet another embodiment, the molecular weight of such compound is at least 1,200 grams per mole.

The compound of this invention preferably has a positive magnetic susceptibility of at least 1,000×10⁻⁶ centimeter-gram-seconds (cgs). As is known to those skilled in the art, magnetic susceptibility is the ratio of the magnetization of a material to the magnetic filed strength. Reference may be had, e.g., to U.S. Pat. No. 3,614,618 (magnetic susceptibility tester), U.S. Pat. No. 3,644,823 (nulling coil apparatus for magnetic susceptibility logging), U.S. Pat. No. 3,657,636 (thermally stable coil assembly for magnetic susceptibility logging), U.S. Pat. No. 3,665,297 (apparatus for determining magnetic susceptibility in a controlled chemical and thermal environment), U.S. Pat. No. 3,758,847 (method and system with voltage cancellation for measuring the magnetic susceptibility of a subsurface earth formation), U.S. Pat. No. 3,758,848 (magnetic susceptibility well logging system), U.S. Pat. No. 3,879,658 (apparatus for measuring magnetic susceptibility), U.S. Pat. No. 3,890,563 (magnetic susceptibility logging apparatus for distinguishing ferromagnetic materials), U.S. Pat. No. 3,980,076 (method for measuring externally of the human body magnetic susceptibility changes), U.S. Pat. No. 4,079,730 (apparatus for measuring externally of the human body magnetic susceptibility changes), U.S. Pat. No. 4,277,750 (induction probe for the measurement of magnetic susceptibility), U.S. Pat. No. 4,359,399 (taggands with induced magnetic susceptibility), U.S. Pat. No. 4,507,613 (method for identifying non-magnetic minerals in earth formations utilizing magnetic susceptibility measurements), U.S. Pat. No. 4,662,359 (use of magnetic susceptibility probes in the treatment of cancer), U.S. Pat. No. 4,701,712 (thermoregulated magnetic susceptibility sensor assembly), U.S. Pat. No. 5,233,992 (MRI method for high liver iron measurement using magnetic susceptibility induced field distortions), U.S. Pat. No. 6,208,884 (noninvasive room temperature instrument to measure magnetic susceptibility variations in body tissue), U.S. Pat. No. 6,321,105 (contrast agents with high magnetic susceptibility), U.S. Pat. No. 6,477,398 (resonant magnetic susceptibility imaging), and the like. The entire disclosure of each of these United States patent applications is hereby incorporated by reference into this specification.

In one embodiment, the compound of this invention has a positive magnetic susceptibility of at least 5,000×10⁻⁶ cgs. In another embodiment, such compound has a positive magnetic susceptibility of at least 10,000×10⁻⁶ cgs.

The compound of this invention is preferably comprised of at least 7 carbon atoms and, more preferably, at least about 10 carbon atoms. In another embodiment, such compound is comprised of at least 13 carbon atoms and at least one aromatic ring; in one aspect of this embodiment, the compound has at least two aromatic rings. In another embodiment, such compound is comprised of at least 17 carbon atoms.

In one embodiment, the compound of this invention is comprised of at least one oxetane ring. As is disclosed, e.g., on page 863 of N. Iving Sax's “Hawley's Condensed Chemical Dictionary,” Eleventh Edition (Van Nostrand Reinhold Company, New York, N.Y., 1987), the oxetane group, also known as “trimethylene oxide), is identified by chemical abstract number CAS: 503-30-0. The oxetane group present in the preferred compound preferably is unsubstituted. In one embodiment, however, one ore more of the ring carbon atoms (either carbon number one, or carbon number two, or carbon number 3), has one or more of its hydrogen atoms substituted by a halogen group (such as chlorine), a lower alkyl group of from 1 to 4 carbon atoms, a lower haloalkyl group of from 1 to 4 carbon atoms, a cyanide group (CN), a hydroxyl group, a carboxyl group, an amino group (wich can be primary, secondary, or teriarary and may also contain from 0 to 6 carbon atoms), a substituted hydroxyl group (such as, e.g., an ether group containing from 1 to 6 carbon atoms), and the like. In one aspect of this embodiment, the substituted oxetane group is 3,3-bis (chlormethyl) oxetane.

In one embodiment, the compound of this invention is comprised of from about 1 to 10 groups of the formula —OB, in which B is selected from the group consisting of hydrogen, alkyl of from about 1 to about 5 carbon atoms, and a moiety of the formula R—(C═O)—O—, wherein R is selected from the group consisting of hydrogen and alkyl of from about 1 to about 6 cabon atoms, and the carbon is bonded to the R moiety, to the double-bonded oxygen, and to the single bonded oxygen, thereby forming what is commonly known as an acetyl group. This acetyl group preferably is linked to a ring structure that is unsaturated and preferably contains from about 6 to about 10 carbon atoms.

In one embodiment, the compound is comprised of two unsaturated ring structures linked by an amide structure, which typically has an acyl group, —CONR₁ —, wherein R₁ is selected from the group consisting of hydrogen lower alkyl of from 1 to about 6 carbon atoms. In one preferred embodiment, the N group is bonded to both to the R₁ group and also to radical that contains at least about 20 carbon atoms and at least about 10 oxygen atoms.

In one embodiment, the compound of this invention contains at least one saturated ring comprising from about 6 to about 10 carbon atoms. By way of illustration, the saturated ring structures may be one or more cyclohexane rings, cyclopheptane rings, cyclooctane rings, cylclononane rings, and/or cylcodecane rings. In one preferred aspect of this embodiment, at least one saturated ring in the compound is bonded to at least one quinine group. Referring to page 990 of the “Hawley's Condensed Chemical Dictionary” described elsewhere in this specification, quinine is 1,4-benzoquinone and is identified as “CAS: 106-51-4.”

In one embodiment, the compound of this invention may comprise a ring structure with one double bond or two double bonds (as opposed to the three double bonds in the aromatic structures). These ring structures may be a partially unsaturated material selected from the group consisting of partially unsaturated cyclohexane, partially unsaturated cyclopheptane, partially unsaturated cyclooctane, partially unstaruated cyclononane, partially unsaturated cyclodecane, and mixtures thereof.

The compound of this invention is also preferably comprised of at least one inorganic atom with a positive magnetic susceptibility of at least 200×10⁻⁶ cgs. Thus, and referring to the “CRC Handbook of Chemistry and Physics,” 63^{rd} Edition (CRC Press, Inc., Boca Raton, Fla., 1982-83), the magnetic susceptibility of elements are described at pages E-118 to E-123. Suitable inorganic (i.e., non-carbon containing) elements with a positive magnetic susceptibility greater than about 200×10⁻⁶ cgs include, e.g., cerium (+5,160×10⁻⁶ cgs), cobalt (+11,000×10⁻⁶ cgs), dysprosium (+89,600×10⁻⁶ cgs), europium (+34,000×10⁻⁶ cgs), gadolinium (+755,000×10⁻⁶ cgs), iron (+13,600×10⁻⁶ cgs), manganese (+529×10⁻⁶ cgs), palladium (+567.4×10⁻⁶ cgs), plutonium (+610×10⁻⁶ cgs), praseodymium (+5010×10⁻⁶ cgs), samarium (+2230×10⁻⁶ cgs), technetium (+250×10⁻⁶ cgs), thulium (+51,444×10⁻⁶ cgs), and the like. In one embodiment, the positive magnetic susceptibility of such element is preferably greater than about +500×10⁻⁶ cgs and, even more preferably, greater than about +1,000×10⁻⁶ cgs.

In one preferred compound, the inorganic atom is radioactive. As is known to those skilled in the art, radioactivity is a phenomenon characterized by spontaneous disintegration of atomic nuclei with emission of corpuscular or electromagnetic radiation.

In another preferred embodiment, one or more inorganic or organic atoms that do not have the specified degree of magnetic suscpeptibility are radioactive. Thus, e.g., the radioactive atom may be, .e.g, radioactive carbon, radioactive hydrogen (tritium), radioactive phosphorus, radioactive sulfur, radioactive potassium, or any other of the atoms that exist is radioactive isotope form.

One preferred class of atoms is the class of radioactive nuclides. As is known to those skilled in the art, radioactive nuclides are atoms disintegrate by emission of corpuscular or electromagnetic radiatons. The rays most commonly emitted are alpha or beta gamma rays. See, e.g., page F-109 of the aforementioned “CRC Handbook of Chemistry and Physics.”

Radioactive nuclides are well known and are described, e.g., in U.S. Pat. No. 4,355,179 (radioactive nuclide labeled propiophenone compounds), U.S. Pat. No. 4,625,118 (device for the elution and metering of a radioactive nuclide), U.S. Pat. No. 5,672,876 (method and apparatus for measuring distribution of radioactive nuclide in a subject), and U.S. Pat. No. 6,607,710 (bisphosphonic acid derivative and compound thereof labeled with radioactive nuclide.). The entire disclosure of each of these United States patents is hereby incorporated by reference into this specification.

Referring again to the aforementioned “CRC Handbook of Chemistry and Physics,” and to pages and in particular to pages B340-B378 thereof, it will be seen that the inorganic atom may be, e.g., cobalt 53, cobalt 54, cobalt 55, cobalt 56, cobalt 57, cobalt 58, cobalt 59, cobalt 60, cobalt 61, cobalt 62, cobalt 63, gadolinium 146, iron 49, iron 51, iron 52, iron 53, iron 54, iron 57, iron 58, iron 59, iron 60, iron 61, iron 62, manganese 50, praseodymium 135, samarium 156, and the like.

The compound of this invention preferably has a magnetic moment of at least about 0.5 Bohr magnetrons per molecule and, more preferably, at least about 1.0 Bohr magnetrons per molecule. In one embodiment, the compound has a magnetic moment of at least about 2 Bohr magnetrons per molecule.

As is known to those skilled in the art, a Bohr magnetron is the amount he/4(pi)mc, wherein he is Plank's constant, e and m are the charge and mass of the electron, c is the speed of light, and pi is equal to about 3.14567. Reference may be had, e.g., to U.S. Pat. Nos. 4,687,331, 4,832,877, 4,849,107, 5,040,373 (“(One Bohr magnetron is equal to 9.273×10-24 Joules/Tesla”), U.S. Pat. Nos. 5,169,944, 5,323,227 (“μo is a constant known as the Bohr magnetron at 9.274×10-21 erg/Gauss”), U.S. Pat. Nos. 5,352,979 6,383,597, 6,725,668, 6,739,137 (“One Bohr magnetron [B is equal to 9.273×10-24 Joules/Tesla”), and the like. The entire disclosure of each of these United States patents is hereby incorporated by reference into this specification.

In one preferred embodiment, the magnetic compound of this invention is water soluble. As is known to those skilled in the art, solubility of one liquid or solid in another is the mass of the substance cotnained in a solution which is in equilibrium with an excess of the substance. Under such conditions, the solution is said to be saturated. Reference may be had, e.g., to page F-95 of the CRC “Handbook of Chemistry and Physics,” 53^{rd} Edition (The Chemical Rubber Company, CRC Press Division, 18901 Cranwood Parkway, Cleveland, Ohio, 44128, 1972-1973).

As used in this specification, the term “water soluble” refers to a solubility of at least 10 micrograms per milliliter and, more preferably, at least 100 micrograms per milliliter; by way of comparison, the solubility of paclitaxel in water is only about 0.4 micrograms per milliliter. One may determine water solubulity by conventional means. Thus, e.g., one may mix 0.5 milliters of water with the compound to be tested under ambient conditions, stir for 18 hours under ambient conditions, filter the slurry thus produced to remove the non-solubulized portion of the fitrand, and calculae how much of the filtrand was solubilized. From this, one can determine the number of micrograms that went into solution.

In one embodiment, the magnetic compound of this invention has a water solubility of at least 500 micrograms per milliliter, and more preferably at least 1,000 micrograms per milliliter. In yet another embodiment, the magnetic compound of this invention has a water solubility of at least 2500 micrograms per milliliter. In yet another embodiment, the magnetic compound of this invention has a water solubility of at least 5,000 micrograms per milliliter. In yet another embodiment, the magnetic compound of this invention has a water solubility of at least 10,000 micrograms per milliliter.

In another embodiment, the magnetic compound of this invention has a water solubility of less than about 10 micrograms per milliliter and, preferably, less than about 1.0 micrograms per milliliter.

Without wishing to be bound to any particular theory, applicants believe that the presence of a hydrophilic group in the compound of their invention helps render such compound water-soluble. Thus, e.g., it is believed that the siderophore group that is present in their preferred compounds aids in creating such water-solubility. As is known to those skilled in the art, a siderophe is one of a number of low molecular weight, iron-containing, or iron binding organic compounds or groups. Siderophores have a stomg affinity for Fe³⁺ (which they chelate) and function in the solubilization and transport of iron. Siderophores are classified as belonging to either the phenol-catechol type (such as enterobactin and agrobactin), or the hydroxyamic acid type (such as ferrichome and mycobactin). Reference may be had, e.g., to page 442 of J. Stenesh's “Dictionary of Biochemistry and Molecular Biology,” Second Edition (John Wiley & Sons, New York, N.Y., 1989).

In one preferred embodiment, the compound of this invention is comprised of one or more siderophore groups bound to a magnetic moiety (such as, e.g., an atom selected from the group consisting of iron, cobalt, nickel, and mixtures thereof).

As will be apparent, the inclusion of other hydrophilic groups into otherwise water-insoluble compounds is contemplated. Thus, by way of illustration and not limitation, and in place of or in addition to such siderophore group, one use hydrophilic groups such as the siderophore group(s) described hereinabove, hydroxyl groups, carboxyl groups, amino groups, organometallic ionic structures, phosphate groups, and the like. In one preferred aspect of this embodiment, the hydrophilic group utilized should preferably be biologically inert.

In one embodiment, the magnetic compound of this invention has an association rate with microtubules of at least 3,500,000/mole/second. The association rate may be determined in accordance with the procedure described in an article by J. F. Diaz et al., “Fast Kinetics of Taxol Binding to Microtubules,” Journal of Biological Chemistry, 278(10) 8407-8455. Reference also may be had, e.g., to a paper by J. R. Strobe et al. appearing in the Journal of Biological Chemistry, 275: 26265-26276 (2000). As is disclosed, e.g., in the Diaz et al. paper, “The kinetics of binding and dissociation of Flutax-1 and Flutax-2 were measured by the change of fluorescence intensity using an SS-51 stopped flow device (High-Tech Scientific, UK) equipped with a fluorescence detetion system, using an excitation wavelenght of 492 and a 530-nm cut-off filter in the emission pathway. The fitting of the kinetic curves was done with a non-linear least squares sfitting program based upon the Marquardt algorithm . . . where pseudo-firt order conditions were used . . . .”

In another embodiment of the invention, the magnetic compound of this invention has a dissociation rate with microubules, as measured in accordance with the procedure desribed in such Diaz et al. paper, of less than about 0.08/second, when measured at a temperature of 37 degrees Celsius and under atmospheric conditions. Thus, in this embodiment, the magnetic compound of this invention binds more durably to microtubules than does paclitaxel, which has a dissociation rate of at least 0.91/second.

In one embodiment, the dissociation rate of the magnetic compound of this invention is less than 0.7/second and, more preferably, less than 0.6/second.

In one embodiment of this invention, the anti-mitotic compound of the invention has the specified degree of water-solubility and of anti-mitotic activity but does not necessarily possess one or more of the magnetic properties described hereinabove.

Other Magnetic Compounds

In another embodiment of this invention, other compounds which are not necessarily anti-mitotic are made magnetic by a process comparable to the process described in this specification for making taxanes magnetic.

In this embodiment, it is preferred to make “magnetic derivatives” of drugs and therapeutic agents. These derivative compounds each preferably have a molecular weight of at least 150 grams per mole, a positive magnetic susceptibility of at least 1,000×10⁻⁶ cgs, and a magnetic moment of at least 0.5 bohr magnetrons, wherein said compound is comprised of at least 7 carbon atoms and at least one, inorganic atom with a positive magnetic susceptibility of at least 200×10⁻⁶ cgs.

Some of the preferred “precursors” used to make these “derivative compounds” are described in the remainder of this section of the specification.

The precursor materials may be either proteinaceous or non-proteinaceous drugs, as they terms are defined in U.S. Pat. No. 5,194,581, the entire disclosure of which is hereby incorporated by reference into this specification. U.S. Pat. No. 5,194,581 discloses “The drugs with which can be incorporated in the compositions of the invention include non-proteinaceous as well as proteinaceous drugs. The term “non-proteinaceous drugs” encompasses compounds which are classically referred to as drugs such as, for example, mitomycin C, daunorubicin, vinblastine, AZT, and hormones. Similar substances are within the skill of the art. The proteinaceous drugs which can be incorporated in the compositions of the invention include immunomodulators and other biological response modifiers. The term “biological response modifiers” is meant to encompass substances which are involved in modifying the immune response in such manner as to enhance the particular desired therapeutic effect, for example, the destruction of the tumor cells. Examples of immune response modifiers include such compounds as lymphokines. Examples of lymphokines include tumor necrosis factor, the interleukins, lymphotoxin, macrophage activating factor, migration inhibition factor, colony stimulating factor and the interferons. Interferons which can be incorporated into the compositions of the invention include alpha-interferon, beta-interferon, and gamma-interferon and their subtypes. In addition, peptide or polysaccharide fragments derived from these proteinaceous drugs, or independently, can also be incorporated. Also, encompassed by the term “biological response modifiers” are substances generally referred to as vaccines wherein a foreign substance, usually a pathogenic organism or some fraction thereof, is used to modify the host immune response with respect to the pathogen to which the vaccine relates. Those of skill in the art will know, or can readily ascertain, other substances which can act as proteinaceous drugs.”

The precursor may be a lectin, as is disclosed in U.S. Pat. No. 5,176,907, the entire disclosure of which is hereby incorporated by reference into this specification. This United States patent discloses “Lectins are proteins, usually isolated from plant material, which bind to specific sugar moieties. Many lectins are also able to agglutinate cells and stimulate lymphocytes. Other therapeutic agents which can be used therapeutically with the biodegradable compositions of the invention are known, or can be easily ascertained, by those of ordinary skill in the art.”

The precursor material may be an amorphous water-soluble pharmaceutical agent, as is disclosed in U.S. Pat. No. 6,117,455, the entire disclosure of which is hereby incorporated by reference into this specification. As is disclosed in the abstract of this patent, there is provided “A sustained-release microcapsule contains an amorphous water-soluble pharmaceutical agent having a particle size of from 1 nm-10 μm and a polymer. The microcapsule is produced by dispersing, in an aqueous phase, a dispersion of from 0.001-90% (w/w) of an amorphous water-soluble pharmaceutical agent in a solution of a polymer having a wt. avg. molecular weight of 2,000-800,000 in an organic solvent to prepare an s/o/w emulsion and subjecting the emulsion to in-water drying.”

In one embodiment, and referring to U.S. Pat. No. 5,420,105 (the entire disclosure of which is hereby incorporated by reference into this specification), the precursor material is selected from the group consisting of an anti-cancer anthracycline antibiotic, cis-platinum, methotrexate, vinblastine, mitoxanthrone ARA-C, 6-mercaptopurine, 6-mercaptoguanosine, mytomycin C and a steroid.

By way of further illustration, the precursor material is selected from the group consisting of antithrombogenic agents, antiplatelet agents, prostaglandins, thrombolytic drugs, antiproliferative drugs, antirejection drugs, antimicrobial drugs, growth factors, and anticalcifying agents.

By way of yet further illustration, the precursor material may, e.g., be any one or more of the therapeutic agents disclosed in column 5 of U.S. Pat. No. 5,464,650. Thus, and referring to such column 5, “The therapeutic substance used in the present invention could be virtually any therapeutic substance which possesses desirable therapeutic characteristics for application to a blood vessel. This can include both solid substances and liquid substances. For example, glucocorticoids (e.g. dexamethasone, betamethasone), heparin, hirudin, tocopherol, angiopeptin, aspirin, ACE inhibitors, growth factors, oligonucleotides, and, more generally, antiplatelet agents, anticoagulant agents, antimitotic agents, antioxidants, antimetabolite agents, and anti-inflammatory agents could be used. Antiplatelet agents can include drugs such as aspirin and dipyridamole. Aspirin is classified as an analgesic, antipyretic, anti-inflammatory and antiplatelet drug. Dypridimole is a drug similar to aspirin in that it has anti-platelet characteristics. Dypridimole is also classified as a coronary vasodilator. Anticoagulant agents can include drugs such as heparin, coumadin, protamine, hirudin and tick anticoagulant protein. Antimitotic agents and antimetabolite agents can include drugs such as methotrexate, azathioprine, vincristine, vinblastine, fluorouracil, adriamycin and mutamycin.”

The precurors material may be one or more of the drugs disclosed in U.S. Pat. No. 5,599,352, the entire disclosure of which is hereby incorporated by reference into this specification. As is disclosed in this patent, “Examples of drugs that are thought to be useful in the treatment of restenosis are disclosed in published international patent application WO 91/12779 “Intraluminal Drug Eluting Prosthesis” which is incorporated herein by reference. Therefore, useful drugs for treatment of restenosis and drugs that can be incorporated in the fibrin and used in the present invention can include drugs such as anticoagulant drugs, antiplatelet drugs, antimetabolite drugs, anti-inflammatory drugs and antimitotic drugs. Further, other vasoreactive agents such as nitric oxide releasing agents could also be used . . . By this method, drugs such as glucocorticoids (e.g. dexamethasone, betamethasone), heparin, hirudin, tocopherol, angiopeptin, aspirin, ACE inhibitors, growth factors, oligonucleotides, and, more generally, antiplatelet agents, anticoagulant agents, antimitotic agents, antioxidants, antimetabolite agents, and anti-inflammatory agents can be applied to a stent . . . .”

By way of yet further illustration, and referring to U.S. Pat. No. 5,605,696 (the entire disclosure of which is hereby incororporated by reference into this specification), the precursor may be a “selected therapeutic drug” that may be, e.g., “ . . . anticoagulant antiplatelet or antithrombin agents such as heparin, D-phe-pro-arg-chloromethylketone (synthetic antithrombin), dipyridamole, hirudin, recombinant hirudin, thrombin inhibitor (available from Biogen), or c7E3 (an antiplatelet drug from Centocore); cytostatic or antiproliferative agents such as angiopeptin (a somatostatin analogue from Ibsen), angiotensin converting enzyme inhibitors such as Captopril (available from Squibb), Cilazapril (available from Hoffman-LaRoche), or Lisinopril (available from Merk); calcium channel blockers (such as Nifedipine), colchicine, fibroblast growth factor (FGF) antagonists, fish oil (omega 3-fatty acid), low molecular weight heparin (available from Wyeth, and Glycomed), histamine antagonists, Lovastatin (an inhibitor of HMG-CoA reductase, a cholesterol lowering drug from Merk), methotrexate, monoclonal antibodies (such as to PDGF receptors), nitroprusside, phosphodiesterase inhibitors, prostacyclin and prostacyclin analogues, prostaglandin inhibitor (available from Glaxo), Seramin (a PDGF antagonist), serotonin blockers, steroids, thioprotease inhibitors, and triazolopyrimidine (a PDGF antagonist). Other therapeutic drugs which may be appropriate include alphainterferon and genetically engineered epithelial cells, for example.”

By way of yet further illustration, and referring to U.S. Pat. No. 5,700,286 (the entire disclosure of which is hereby incorporated by reference into this specification), precursor material may be a therapeutic agent or drug “ . . . including, but not limited to, antiplatelets, antithrombins, cytostatic and antiproliferative agents, for example, to reduce or prevent restenosis in the vessel being treated. The therapeutic agent or drug is preferably selected from the group of therapeutic agents or drugs consisting of sodium heparin, low molecular weight heparin, hirudin, argatroban, forskolin, vapiprost, prostacyclin and prostacyclin analogues, dextran, D-phe-pro-arg-chloromethylketone, dipyridamole, glycoprotein IIb/IIIa platelet membrane receptor antibody, recombinant hirudin, thrombin inhibitor, angiopeptin, angiotensin converting enzyme inhibitors, (such as Captopril, available from Squibb; Cilazapril, available for Hoffman-La Roche; or Lisinopril, available from Merck) calcium channel blockers, colchicine, fibroblast growth factor antagonists, fish oil, omega 3-fatty acid, histamine antagonists, HMG-CoA reductase inhibitor, methotrexate, monoclonal antibodies, nitroprusside, phosphodiesterase inhibitors, prostaglandin inhibitor, seramin, serotonin blockers, steroids, thioprotease inhibitors, triazolopyrimidine and other PDGF antagonists, alpha-interferon and genetically engineered epithelial cells, and combinations thereof.”

By way of yet further illustration, and referring to U.S. Pat. No. 5,900,433 (the entire disclosure of which is hereby incorporated by reference into this specification), the precursor material may be a congener of an endothelium-derived bioactive composition of matter. This congener is discussed in column 7 of the patent, wherein it is disclosed that “We have discovered that administration of a congener of an endothelium-derived bioactive agent, more particularly a nitrovasodilator, representatively the nitric oxide donor agent sodium nitroprusside, to an extravascular treatment site, at a therapeutically effective dosage rate, is effective for abolishing CFR's while reducing or avoiding systemic effects such as supression of platelet function and bleeding . . . congeners of an endothelium-derived bioactive agent include prostacyclin, prostaglandin E1, and a nitrovasodilator agent. Nitrovasodilater agents include nitric oxide and nitric oxide donor agents, including L-arginine, sodium nitroprusside and nitroglycycerine.”

By way of yet further illustration, the precursor material may be heparin. As is disclosed in U.S. Pat. No. 6,120,536 (the entire disclosure of which is hereby incorporated by reference into this specification), “While heparin is preferred as the incorporated active material, agents possibly suitable for incorporation include antithrobotics, anticoagulants, antibiotics, antiplatelet agents, thorombolytics, antiproliferatives, steroidal and non-steroidal antinflammatories, agents that inhibit hyperplasia and in particular restenosis, smooth muscle cell inhibitors, growth factors, growth factor inhibitors, cell adhesion inhibitors, cell adhesion promoters and drugs that may enhance the formation of healthy neointimal tissue, including endothelial cell regeneration.”

By way of yet further illustration, and referring to U.S. Pat. No. 6,624,138 (the entire disclosure of which is hereby incorporated by reference into this specification), the precursor material may be one or more of the drugs described in this patent. Thus, and referring to columns 9 et seq. of such patent, “Straub et al. in U.S. Pat. No. 6,395,300 discloses a wide variety of drugs that are useful in the methods and compositions described herein, entire contents of which, including a variety of drugs, are incorporated herein by reference. Drugs contemplated for use in the compositions described in U.S. Pat. No. 6,395,300 and herein disclosed include the following categories and examples of drugs and alternative forms of these drugs such as alternative salt forms, free acid forms, free base forms, and hydrates: analgesics/antipyretics. (e.g., aspirin, acetaminophen, ibuprofen, naproxen sodium, buprenorphine, propoxyphene hydrochloride, propoxyphene napsylate, meperidine hydrochloride, hydromorphone hydrochloide, morphine, oxycodone, codeine, dihydrocodeine bitartrate, pentazocine, hydrocodone bitartrate, levorphanol, diflunisal, trolamine salicylate, nalbuphine hydrochloride, mefenamic acid, butorphanol, choline salicylate, butalbital, phenyltoloxamine citrate, diphenhydramine citrate, methotrimeprazine, cinnamedrine hydrochloride, and meprobamate); antiasthamatics (e.g., ketotifen and traxanox); antibiotics (e.g., neomycin, streptomycin, chloramphenicol, cephalosporin, ampicillin, penicillin, tetracycline, and ciprofloxacin); antidepressants (e.g., nefopam, oxypertine, doxepin, amoxapine, trazodone, amitriptyline, maprotiline, phenelzine, desipramine, nortriptyline, tranylcypromine, fluoxetine, doxepin, imipramine, imipramine pamoate, isocarboxazid, trimipramine, and protriptyline); antidiabetics (e.g., biguanides and sulfonylurea derivatives); antifungal agents (e.g., griseofulvin, ketoconazole, itraconizole, amphotericin B, nystatin, and candicidin); antihypertensive agents (e.g., propanolol, propafenone, oxyprenolol, nifedipine, reserpine, trimethaphan, phenoxybenzamine, pargyline hydrochloride, deserpidine, diazoxide, guanethidine monosulfate, minoxidil, rescinnamine, sodium nitroprusside, rauwolfia serpentina, alseroxylon, and phentolamine); anti-inflammatories (e.g., (non-steroidal) indomethacin, ketoprofen, flurbiprofen, naproxen, ibuprofen, rarnifenazone, piroxicam, (steroidal) cortisone, dexamethasone, fluazacort, celecoxib, rofecoxib, hydrocortisone, prednisolone, and prednisone); antineoplastics (e.g., cyclophosphamide, actinomycin, bleomycin, daunorubicin, doxorubicin, epirubicin, mitomycin, methotrexate, fluorouracil, carboplatin, carmustine (BCNU), methyl-CCNU, cisplatin, etoposide, camptothecin and derivatives thereof, phenesterine, paclitaxel and derivatives thereof, docetaxel and derivatives thereof, vinblastine, vincristine, tamoxifen, and piposulfan); antianxiety agents (e.g., lorazepam, buspirone, prazepam, chlordiazepoxide, oxazepam, clorazepate dipotassium, diazepam, hydroxyzine pamoate, hydroxyzine hydrochloride, alprazolam, droperidol, halazepam, chlormezanone, and dantrolene); immunosuppressive agents (e.g., cyclosporine, azathioprine, mizoribine, and FK506 (tacrolimus)); antimigraine agents (e.g., ergotamine, propanolol, isometheptene mucate, and dichloralphenazone); sedatives/hypnotics (e.g., barbiturates such as pentobarbital, pentobarbital, and secobarbital; and benzodiazapines such as flurazepam hydrochloride, triazolam, and midazolam); antianginal agents (e.g., beta-adrenergic blockers; calcium channel blockers such as nifedipine, and diltiazem; and nitrates such as nitroglycerin, isosorbide dinitrate, pentearythritol tetranitrate, and erythrityl tetranitrate); antipsychotic agents (e.g., haloperidol, loxapine succinate, loxapine hydrochloride, thioridazine, thioridazine hydrochloride, thiothixene, fluphenazine, fluphenazine decanoate, fluphenazine enanthate, trifluoperazine, chlorpromazine, perphenazine, lithium citrate, and prochlorperazine); antimanic agents (e.g., lithium carbonate); antiarrhythmics (e.g., bretylium tosylate, esmolol, verapamil, amiodarone, encainide, digoxin, digitoxin, mexiletine, disopyramide phosphate, procainamide, quinidine sulfate, quinidine gluconate, quinidine polygalacturonate, flecainide acetate, tocainide, and lidocaine); antiarthritic agents (e.g., phenylbutazone, sulindac, penicillanine, salsalate, piroxicam, azathioprine, indomethacin, meclofenamate, gold sodium thiomalate, ketoprofen, auranofin, aurothioglucose, and tolmetin sodium); antigout agents (e.g., colchicine, and allopurinol); anticoagulants (e.g., heparin, heparin sodium, and warfarin sodium); thrombolytic agents (e.g., urokinase, streptokinase, and alteplase); antifibrinolytic agents (e.g., aminocaproic acid); hemorheologic agents (e.g., pentoxifylline); antiplatelet agents (e.g., aspirin); anticonvulsants (e.g., valproic acid, divalproex sodium, phenyloin, phenyloin sodium, clonazepam, primidone, phenobarbitol, carbamazepine, amobarbital sodium, methsuximide, metharbital, mephobarbital, mephenyloin, phensuximide, paramethadione, ethotoin, phenacemide, secobarbitol sodium, clorazepate dipotassium, and trimethadione); antiparkinson agents (e.g., ethosuximide); antihistamines/antipruritics (e.g., hydroxyzine, diphenhydramine, chlorpheniramine, brompheniramine maleate, cyproheptadine hydrochloride, terfenadine, clemastine fumarate, triprolidine, carbinoxamine, diphenylpyraline, phenindamine, azatadine, tripelennamine, dexchlorpheniramine maleate, methdilazine; agents useful for calcium regulation (e.g., calcitonin, and parathyroid hormone); antibacterial agents (e.g., amikacin sulfate, aztreonam, chloramphenicol, chloramphenicol palirtate, ciprofloxacin, clindamycin, clindamycin palmitate, clindamycin phosphate, metronidazole, metronidazole hydrochloride, gentamicin sulfate, lincomycin hydrochloride, tobramycin sulfate, vancomycin hydrochloride, polymyxin B sulfate, colistimethate sodium, and colistin sulfate); antiviral agents (e.g., interferon alpha, beta or gamma, zidovudine, amantadine hydrochloride, ribavirin, and acyclovir); antimicrobials (e.g., cephalosporins such as cefazolin sodium, cephradine, cefaclor, cephapirin sodium, ceftizoxime sodium, cefoperazone sodium, cefotetan disodium, cefuroxime e azotil, cefotaxime sodium, cefadroxil monohydrate, cephalexin, cephalothin sodium, cephalexin hydrochloride monohydrate, cefamandole nafate, cefoxitin sodium, cefonicid sodium, ceforanide, ceftriaxone sodium, ceftazidime, cefadroxil, cephradine, and cefuroxime sodium; penicillins such as ampicillin, amoxicillin, penicillin G benzathine, cyclacillin, ampicillin sodium, penicillin G potassium, penicillin V potassium, piperacillin sodium, oxacillin sodium, bacampicillin hydrochloride, cloxacillin sodium, ticarcillin disodium, azlocillin sodium, carbenicillin indanyl sodium, penicillin G procaine, methicillin sodium, and nafcillin sodium; erythromycins such as erythromycin ethylsuccinate, erythromycin, erythromycin estolate, erythromycin lactobionate, erythromycin stearate, and erythromycin ethylsuccinate; and tetracyclines such as tetracycline hydrochloride, doxycycline hyclate, and minocycline hydrochloride, azithromycin, clarithromycin); anti-infectives (e.g., GM-CSF); bronchodilators (e.g., sympathomimetics such as epinephrine hydrochloride, metaproterenol sulfate, terbutaline sulfate, isoetharine, isoetharine mesylate, isoetharine hydrochloride, albuterol sulfate, albuterol, bitolterolmesylate, isoproterenol hydrochloride, terbutaline sulfate, epinephrine bitartrate, metaproterenol sulfate, epinephrine, and epinephrine bitartrate; anticholinergic agents such as ipratropium bromide; xanthines such as aminophylline, dyphylline, metaproterenol sulfate, and aminophylline; mast cell stabilizers such as cromolyn sodium; inhalant corticosteroids such as beclomethasone dipropionate (BDP), and beclomethasone dipropionate monohydrate; salbutamol; ipratropium bromide; budesonide; ketotifen; salmeterol; xinafoate; terbutaline sulfate; triamcinolone; theophylline; nedocromil sodium; metaproterenol sulfate; albuterol; flunisolide; fluticasone proprionate; steroidal compounds and hormones (e.g., androgens such as danazol, testosterone cypionate, fluoxymesterone, ethyltestosterone, testosterone enathate, methyltestosterone, fluoxymesterone, and testosterone cypionate; estrogens such as estradiol, estropipate, and conjugated estrogens; progestins such as methoxyprogesterone acetate, and norethindrone acetate; corticosteroids such as triamcinolone, betamethasone, betamethasone sodium phosphate, dexamethasone, dexamethasone sodium phosphate, dexamethasone acetate, prednisone, methylprednisolone acetate suspension, triamcinolone acetonide, methylprednisolone, prednisolone sodium phosphate, methylprednisolone sodium succinate, hydrocortisone sodium succinate, triamcinolone hexacetonide, hydrocortisone, hydrocortisone cypionate, prednisolone, fludrocortisone acetate, paramethasone acetate, prednisolone tebutate, prednisolone acetate, prednisolone sodium phosphate, and hydrocortisone sodium succinate; and thyroid hormones such as levothyroxine sodium); hypoglycemic agents (e.g., human insulin, purified beef insulin, purified pork insulin, glyburide, chlorpropamide, glipizide, tolbutamide, and tolazamide); hypolipidemic agents (e.g., clofibrate, dextrothyroxine sodium, probucol, pravastitin, atorvastatin, lovastatin, and niacin); proteins (e.g., DNase, alginase, superoxide dismutase, and lipase); nucleic acids (e.g., sense or anti-sense nucleic acids encoding any therapeutically useful protein, including any of the proteins described herein); agents useful for erythropoiesis stimulation (e.g., erythropoietin); antiulcer/antireflux agents (e.g., famotidine, cimetidine, and ranitidine hydrochloride); antinauseants/antiemetics (e.g., meclizine hydrochloride, nabilone, prochlorperazine, dimenhydrinate, promethazine hydrochloride, thiethylperazine, and scopolamine); as well as other drugs useful in the compositions and methods described herein include mitotane, halonitrosoureas, anthrocyclines, ellipticine, ceftriaxone, ketoconazole, ceftazidime, oxaprozin, albuterol, valacyclovir, urofollitropin, famciclovir, flutamide, enalapril, mefformin, itraconazole, buspirone, gabapentin, fosinopril, tramadol, acarbose, lorazepan, follitropin, glipizide, omeprazole, fluoxetine, lisinopril, tramsdol, levofloxacin, zafirlukast, interferon, growth hormone, interleukin, erythropoie tin, granulocyte stimulating factor, nizatidine, bupropion, perindopril, erbumine, adenosine, alendronate, alprostadil, benazepril, betaxolol, bleomycin sulfate, dexfenfluramine, diltiazem, fentanyl, flecainid, gemcitabine, glatiramer acetate, granisetron, lamivudine, mangafodipir trisodium, mesalamine, metoprolol fumarate, metronidazole, miglitol, moexipril, monteleukast, octreotide acetate, olopatadine, paricalcitol, somatropin, sumatriptan succinate, tacrine, verapamil, nabumetone, trovafloxacin, dolasetron, zidovudine, finasteride, tobramycin, isradipine, tolcapone, enoxaparin, fluconazole, lansoprazole, terbinafine, pamidronate, didanosine, diclofenac, cisapride, venlafaxine, troglitazone, fluvastatin, losartan, imiglucerase, donepezil, olanzapine, valsartan, fexofenadine, calcitonin, and ipratropium bromide. These drugs are generally considered to be water soluble.” Any of these water-soluble drugs may be used as precursors in the process of this invention to make a composition with the desired magnetic properties.

As is also disclosed in U.S. Pat. No. 6,624,138, “Preferred drugs useful in the present invention may include albuterol, adapalene, doxazosin mesylate, mometasone furoate, ursodiol, amphotericin, enalapril maleate, felodipine, nefazodone hydrochloride, valrubicin, albendazole, conjugated estrogens, medroxyprogesterone acetate, nicardipine hydrochloride, zolpidem tartrate, amlodipine besylate, ethinyl estradiol, omeprazole, rubitecan, amlodipine besylate/benazepril hydrochloride, etodolac, paroxetine hydrochloride, paclitaxel, atovaquone, felodipine, podofilox, paricalcitol, betamethasone dipropionate, fentanyl, pramipexole dihydrochloride, Vitamin D3 and related analogues, finasteride, quetiapine fumarate, alprostadil, candesartan, cilexetil, fluconazole, ritonavir, busulfan, carbamazepine, flumazenil, risperidone, carbemazepine, carbidopa, levodopa, ganciclovir, saquinavir, amprenavir, carboplatin, glyburide, sertraline hydrochloride, rofecoxib carvedilol, halobetasolproprionate, sildenafil citrate, celecoxib, chlorthalidone, imiquimod, simvastatin, citalopram, ciprofloxacin, irinotecan hydrochloride, sparfloxacin, efavirenz, cisapride monohydrate, lansoprazole, tamsulosin hydrochloride, mofafinil, clarithromycin, letrozole, terbinafine hydrochloride, rosiglitazone maleate, diclofenac sodium, lomefloxacin hydrochloride, tirofiban hydrochloride, telmisartan, diazapam, loratadine, toremifene citrate, thalidomide, dinoprostone, mefloquine hydrochloride, trandolapril, docetaxel, mitoxantrone hydrochloride, tretinoin, etodolac, triamcinolone acetate, estradiol, ursodiol, nelfinavir mesylate, indinavir, beclomethasone dipropionate, oxaprozin, flutamide, famotidine, nifedipine, prednisone, cefuroxime, lorazepam, digoxin, lovastatin, griseofulvin, naproxen, ibuprofen, isotretinoin, tamoxifen citrate, nimodipine, amiodarone, and alprazolam. Specific non-limiting examples of some drugs that fall under the above categories include paclitaxel, docetaxel and derivatives, epothilones, nitric oxide release agents, heparin, aspirin, coumadin, PPACK, hirudin, polypeptide from angiostatin and endostatin, methotrexate, 5-fluorouracil, estradiol, P-selectin Glycoprotein ligand-1 chimera, abciximab, exochelin, eleutherobin and sarcodictyin, fludarabine, sirolimus, tranilast, VEGF, transforming growth factor (TGF)-beta, Insulin-like growth factor (IGF), platelet derived growth factor (PDGF), fibroblast growth factor (FGF), RGD peptide, beta or gamma ray emitter (radioactive) agents, and dexamethasone, tacrolimus, actinomycin-D, batimastat etc.” These drugs also may be used in the process of this invention to make magnetic compositons.

Another Preferred Compound of the Invention

In another embodiment of this invention, there is provided a compound that, in spite of having a molecular weight in excess of 550, still has a water solubility in excess of about 10 micrograms per milliliter. In particular, there is provided a compound with a molecular weight of at least about 550, a water solubility of at least about 10 micrograms per milliliter, a pKa dissociation constant of from about 1 to about 15, and a partition coefficient of from about 1.0 to about 50.

The compound of this embodiment of the invention has a molecular weight of at least about 550. In one embodiment, this compound has a molecular weight of at least about 700.

The water solubility of this compound is at least about 1 micrograms per milliliter and, more preferably, at least about 10 micrograms per milliliter. In one embodiment, such compound has a water solubility of at least about 100 micrograms per milliliter. In yet another embodiment, such compound has a water solubility of at least about 1,000 micrograms per milliliter.

The compound of this embodiment of the invention has a pKa dissociation constant of from about 1 to about 15. As used herein, the term “pKa dissociation constant” is equal to −log Kₐ, wherein Kₐ is equal to [H₃O⁺][A⁻]/[HA], wherein the square brackets ([ ]) indicate concentration, and wherein A is the counterion. Reference may be had, e.g., to pages 327-328 of Maitland Jones, Jr.'s “Organic Chemistry” (W.M. Norton & Company, New York, N.Y., 1997). Reference may also be had, e.g., to U.S. Pat. Nos. 5,036,164; 5,025,063; 5,767,066; 5,155,162; 5,132,000; and 5,079,134. The entire disclosure of each of these United States patents is hereby incorporated by reference into this specification.

As is known to those skilled in the art, and as is disclosed at pages 39 et seq. of Stephen H. Curry et al.'s “Manual of Laboratory Phamaconkinetics” (John Wiley & Sons, New York, N.Y., 1983), “Many drugs are weak acids and/or bases. The degree of ionization will influence the absorption, distribution, and excretion in vivo, the solubility at a given pH, the distribution of the drug between aqueous and organic pahses the choice of pH in liquid chromatographic separations, etc . . . . From the above it follows that the pH at which the compound is 50 percent ionized is equal to the pKₐ. To determine a value of pKₐ the relative concentrations of ionized and non-ionized forms msut be known at a particular pH. Several methods are available, including potentiometric titration, conductimetry, solubility, and spectrometery . . . .”

The compound of this embodiment of the invention preferably has a partition coefficient of from about 1.0 to about 50. This partition coefficient is also dicussed at pages 41 et seq. of the aforementioned Curry book, wherein it is disclosed that: “When a solute is distributed between two immiscible phases, 1 and 2, the ratio of the activities of the solute in the phases is constant. If the solutions are dilute and ideal behavior is assumed, then the ratio of the concentration of the solute will be constant . . . . The constant is known as the partition (or distribution) coefficient . . . . The convention with regard to which phase is classed as 1 and which is as 2 is not entirely clear. Usually, partition coefficients are defined as the concentration in the organic phase divided by the concentration in the aqueous phase.”

It is preferred to measure the partition coefficient between water and octane. Means for measuring the partition coefficient are well known to those skilled in the art and are described, e.g., in the patent literature. Reference may be had, e.g., to U.S. Pat. Nos. 6,660,288; 6,645,479; 6,585,953; 6,583,136; 6,500,995; 6,475,961; 6.369.001; 6,362,158; 6,315,907; 6,310,013; 6,271,665; 6,218,378; 6,203,817; 6,156,826; 6,124,086; 6,071,409; 6,045,835; 6,042,792; 5,874,481; 5,763,146; 5,555,747; 5,252,320 (complexes having a partition coefficient above 300); U.S. Pat. Nos. 5,254,342; 5,252,320; 5,164,189; 5,071,769; 5,041,523; 5,013,556; 5,011,982; 5,011,967; 4,986,917; 4,980,453; 4,957,862; 4,940,654; 4,886,656; 4,859,584; 4,762,701; 4,746,745; 4,743,550 (method for improving the partition coefficient in enzyme containing systems having at least two phases), U.S. Pat. Nos. 4,736,016; 4,721,730; 4,699,924; 4,619,939; 4,420,473; 4,371,540; 4,363,793; and the like. The entire disclosure of each of these United States patents is hereby incorporated by reference into this specification.

In one embodiment, the compound of this invention has a tumor uptake of at least about 10 percent and, more preferably, at least about 20 percent. In one embodiment, the tumor uptake is at least about 30 percent. In yet another embodiment, the tumor uptake is at least about 50 percent. In yet another embodiment, the tumor uptake is at least about 70 percent.

Tumor uptake is the extent to which the compound is selectively taken up by tumors from blood. It may be determined by dissolving 1 milligram of the compound to be tested in 1 milliliter of “Cremophor EL,” a 1:1 (volume/volume) mixture of anhydrous ethanol and polyethoxylated castor oil. For a discussion of such “Cremophor EL,” reference may be had, e.g., to U.S. Pat. No. 5,591,715 (methods and compositions for reducing multidrug resistance), U.S. Pat. No. 5,686,488 (polyethoxylated castor oil products as anti-inflammatory agents), U.S. Pat. No. 5,776,891 (compositions for reducing multidrug resistance), and the like. The entire disclosure4 of each of these United States patents is hereby incorporated by reference into this specification.

The mixture of the compound to be tested and “Cremophor EL” is injected ito the blood supply (artery) of a laboratory rat, near the tumor. Thirty seconds later the rate is sacrificed, the tumor is removed, and it and the blood are analyzed for the presence of the compound. Both the arterial blood and the venous drainage beyond the tumor are analyzed. The percent tumor uptake is equal to ([Cₐ−Cᵥ]/Cₐ)×100, wherein Cₐ is the concentration of the compound in the arterial blood, and Cᵥ is the concentration of the compound in the venous blood.

Other conventional means may be used to determine the tumor uptake. Reference may be had, e.g., to U.S. Pat. Nos. 4,448,762; 5,077,034; 5,094,835; 5,135,717; 5,166,944; 5,284,831; 5, 5,391,547; 399,338; 5,474,772; 5,516,940; 5,578,287; 5,595,738; 5,601,800; 5,608,060; 5,616,690; 5,624,798; 5,624,896; 5,683,873; 5,688,501; 5,753,262; 5,762,909; 5,783,169; 5,810,888; 5,811,073; 5,820,873; 5,847,121; 5,869,248; 5,877,162; 5,891,689; 5,902,604; 5,911,969; 5,914,312; 5,955,605; 5,965,598; 5,976,535; 5,976,874;6,008,319; 6,022,522; 6,022,966; 6,025,165; 6,027,725; 6,057,153; 6,074,626; 6,103,889; 6,121,424; 6,165,441; 6,171,577; 6,172,045; 6,197,333; 6,217,869; 6,217,886; 6,235,264; 6,242,477; 6,331,287; 6,348,214; 6,358,490; 6,403,096; 6,426,400; 6,436,708; 6,441,158; 6,458,336; 6,498,181; 6,515,110; 6,537,521; 6,610,478; 6,617,135; 6,620,805; 6,624,187; 6,723,318; 6,734,171; 6,685,915; and the like. The entire disclosure of each of these United States patents is hereby incorporated by reference into this specification.

Guided Delivery of the Compounds of this Invention

In one preferred embodiment, the magnetic properties of the anti-mitotic compound of this invention are used in order to preferentially deliver such compound to a specified site. In another embodiment, the magnetic properties of the compounds and compositions of this invention which are not necessarily anti-mitotic but have the desired magnetic properties also may be used to deliver such compounds and/or compositions to a desired site.

Thus, by way of illustration, one may guide delivery of the compound of this invention with conventional magnetic focusing means. In one aspect of this embodiment, a magnetic field of a specified strength is focused onto a desired therapeutic site, such as a tumor to be treated, whereby the compound is selectively drawn to the therapeutic site and binds with tubulin moleuces at the site. In one embodiment, the focused magnetic field has a field strength of at least about 6 Tesla in order to cause microtubules to move linearly. The magnetic field may, e.g., be focused for a period of at least about 30 minutes following the administration of the compound of this invention.

One may use any of the conventional magnetic field generators known to those skilled in the art to produce such a magnetic field. Thus, e.g., one may use one or more of the magnetic field generators disclosed in U.S. Pat. Nos. 6,503,364, 6,377,149 (magnetic field generator for magnetron plasma generation), U.S. Pat. No. 6,353,375 (magnetostatic wave device), U.S. Pat. No. 6,340,888 (magnetic field generator for MRI), U.S. Pat. Nos. 6,336,989, 6,335,617 (device for calibrating a magnetic field generator), U.S. Pat. Nos. 6,313,632, 6,297,634, 6,275,128, 6,246,066 (magnetic field generator and charged particle beam irradiator), U.S. Pat. No. 6,114,929 (magnetostatic wave device), U.S. Pat. No. 6,099,459 (magnetic field generating device and method of generating and applying a magnetic field), U.S. Pat. Nos. 5,795,212, 6,106,380 (deterministic magnetorheological finishing), U.S. Pat. No. 5,839,944 (apparatus for deterministic magnetorheological finishing), U.S. Pat. No. 5,971,835 (system for abrasive jet shaping and polishing of a surface using a magnetorheological fluid), U.S. Pat. Nos. 5,951,369, 6,506,102 (system for magnetorheological finishing of substrates), U.S. Pat. Nos. 6,267,651, 6,309,285 (magnetic wiper), U.S. Pat. Nos. 5,929,732 and 6,488,615 I (which describe devices and methods for creating a high intensity magnetic field for magnetically guiding a anti-mitotic compound to a predetermined site within a biological organism), and the like. The entire disclosure of each of these United States patents is hereby incorporated by reference into this specification.

The Use of Externally Applied Energy to Affect an Implanted Medical Device

The prior art discloses many devices in which an externally applied electromagnetic field (i.e., a field originating outside of a biological organism, such as a human body) is generated in order to influence one or more implantable devices disposed within the biological organism; these may be used in conjunction with anti-mitotic compound of this invention. Some of these devices are described below.

U.S. Pat. No. 3,337,776 describes a device for producing controllable low frequency magnetic fields; the entire disclosure of this patent is hereby incorporated by reference into this specification. Thus, e.g., claim 1 of this patent describes a biomedical apparatus for the treatment of a subject with controllable low frequency magnetic fields, comprising solenoid means for creating the magnetic field. These low-frequency magnetic fields may be used to affect the anti-mitotic compounds of this invention, and/or tubulin and/or microtubules and/or other moieties.

U.S. Pat. No. 3,890,953 also discloses an apparatus for promoting the growth of bone and other body tissues by the application of a low frequency alternating magnetic field; the entire disclosure of this United States patent is hereby incorporated by reference into this specification. This patent claims “In an electrical apparatus for promoting the growth of bone and other body tissues by the application thereto of a low frequency alternating magnetic field, such apparatus having current generating means and field applicator means, the improvement wherein the applicator means comprises a flat solenoid coil having an axis about which the coil is wound and composed of a plurality of parallel and flexible windings, each said winding having two adjacent elongate portions and two 180° coil bends joining said elongate portions together, said coil being flexible in the coil plane in the region of said elongate portion for being bent into a U-shape, said coil being bent into such U-shape about an axis parallel to the coil axis and adapted for connection to a source of low frequency alternating current.” These low-frequency magnetic fields may be used to affect the anti-mitotic compounds of this invention, and/or tubulin and/or microtubules and/or other moieties.

The device of U.S. Pat. No. 3,890,953 is described, in part, at lines 52 et seq. of column 2, wherein it is disclosed that: “.The apparatus shown diagrammatically in FIG. 1 comprises a AC generator 10, which supplies low frequency AC at the output terminals 12. The frequency of the AC lies below 150 Hz, for instance between 1 and 50 or 65 Hz. It has been found particularly favorable to use a frequency range between 5 or 10 and 30 Hz, for example 25 Hz. The half cycles of the alternating current should have comparatively gently sloping leading and trailing flanks (rise and fall times of the half cycles being for example in the order of magnitude of a quarter to an eighth of the length of a cycle); the AC can thus be a sinusoidal current with a low non-linear distortion, for example less than 20 percent, or preferably less than 10 percent, or a triangular wave current.”

U.S. Pat. No. 4,095,588 discloses a “vascular cleansing device” adapted to “ . . . effect motion of the red corpuscles in the blood stream of a vascular system . . . whereby these red cells may cleanse the vascular system by scrubbing the walls thereof . . . ;” the entire disclosure of this United States patent is hereby incorporated by reference into this specification. This patent claims (in claim 3) “A means to propel a red corpuscle in a vibratory and rotary fashion, said means comprising an electronic circuit and magnetic means including: a source of electrical energy; a variable oscillator connected to said source; a binary counter means connected to said oscillator to produce sequential outputs; a plurality of deflection amplifier means connected to be operable by the outputs of said binary counter means in a sequential manner, said amplifier means thereby controlling electrical energy from said source; a plurality of separate coils connected in separate pairs about an axis in series between said deflection amplifier means and said source so as to be sequentially operated in creating an electromagnetic field from one coil to the other and back again and thence to adjacent separate coils for rotation of the electromagnetic field from one pair of coils to another; and a table within the space encircled by said plurality of coils, said table being located so as to place a person along the axis such that the red corpuscles of the person's vascular system are within the electromagnetic field between the coils creating same.” The energy used to affect such red blood corpuscles may also be used affect the anti-mitotic compounds of this invention, and/or tubulin and/or microtubules and/or other moieties.

U.S. Pat. No. 4,323,075 discloses an implantable defibrillator with a rechargeable power supply; the entire disclosure of this patent is hereby incorporated by reference into this specification. Claim 1 of this patent describes “A fully implantable power supply for use in a fully implantable defibrillator having an implantable housing, a fibrillation detector for detecting fibrillation of the heart of a recipient, an energy storage and discharge device for storing and releasing defibrillation energy into the heart of the recipient and an inverter for charging the energy storage and discharge device in response to detection of fibrillation by the fibrillation detector, the inverter requiring a first level of power to be operational and the fibrillation detector requiring a second level of power different from said first level of power to be operational, said power supply comprising: implantable battery means positioned within said implantable housing, said battery means including a plurality of batteries arranged in series, each of said batteries having a pair of output terminals, each of said batteries producing a distinctly multilevel voltage across its pair of output terminals, said voltage being at a first level when the battery is fully charged and dropping to a second level at some point during the discharge of the battery; and implantable circuit means positioned within said implantable housing, said circuit means for creating a first conductive path betwen said serially-connected batteries and said fibrillation detector to provide said fibrillation detector with said second level of power, and for creating a second conductive path between said inverter and said battery means by placing only the batteries operating at said first level voltage in said second conductive path, and excluding the remaining batteries from said second conductive path to provide said inverter with said first level of power.” The power supply of this patent may be used to power, e.g., one or more magnetic focusing devices.

U.S. Pat. No. 4,340,038 discloses an implanted medical system comprised of magnetic field pick-up means for converting magnetic energy to electrical energy; the entire disclosure of this patent is hereby incorporated by reference into this specification. One may use the electrical energy produced by such pick-up means to affect the anti-mitotic compounds of this invention, and/or tubulin and/or microtubules and/or other moieties. Such energy may also be used to power an implanted magnetic focusing device.

In column 1 of U.S. Pat. No. 4,340,038, at lines 12 et seq., it is disclosed that “Many types of implantable devices incorporate a self-contained transducer for converting magnetic energy from an externally-located magnetic field generator to energy usable by the implanted device. In such a system having an implanted device and an externally-located magnetic field generator for powering the device, sizing and design of the power transfer system is important. In order to properly design the power transfer system while at the same time avoiding overdesign, the distance from the implanted device to the magnetic field generator must be known. However for some types of implanted devices the depth of the implanted device in a recipient's body is variable, and is not known until the time of implantation by a surgeon. One example of such a device is an intracranial pressure monitoring device (ICPM) wherein skull thickness varies considerably between recipients and the device must be located so that it protrudes slightly below the inner surface of the skull and contacts the dura, thereby resulting in a variable distance between the top of the implanted device containing a pick-up coil or transducer and the outer surface of the skull. One conventional technique for accommodating an unknown distance between the magnetic field generator and the implanted device includes increasing the transmission power of the external magnetic field generator. However this increased power can result in heating of the implanted device, the excess heat being potentially hazardous to the recipient. A further technique has been to increase the diameter of the pick-up coil in the implanted device. However, physical size constraints imposed on many implanted devices such as the ICPM are critical; and increasing the diameter of the pick-up coil is undesirable in that it increases the size of the orifice which must be formed in the recipient's skull. The concentrator of the present invention solves the above problems by concentrating magnetic lines of flux from the magnetic generator at the implanted pick-up coil, the concentrator being adapted to accommodate distance variations between the implanted device and the magnetic field generator.’

Claim 1 of U.S. Pat. No. 4,340,038 describes “In a system including an implanted device having a magnetic field pick-up means for converting magnetic energy to electrical energy for energizing said implanted device, and an external magnetic field generator located so that magnetic lines of flux generated thereby intersect said pick-up means, a means for concentrating a portion of said magnetic lines of flux at said pick-up means comprising a metallic slug located between said generator and said pick-up means, thereby concentrating said magnetic lines of flux at said pick-up means. “Claim 5 of this patent further describes the pick-up means as comprising “ . . . a magnetic pick-up coil and said slug is formed in the shape of a truncated cone and oriented so that a plane defined by the smaller of said cone end surfaces is adjacent to said substantially parallel to a plane defined by said magnetic pick-up coil.” In one embodiment, such pick-up means may be located near the site to be treated (such as a tumor) and may be used to affect the tumor by, e.g., hyperthermia treatement.

U.S. Pat. No. 4,361,153 discloses an implantable telemetry system; the entire disclosure of such United States patent is hereby incorporated by reference into this specification. Such an implantable telemetry system, equipped with a multiplicity of sensors, may be used to report how These the anti-mitotic compounds of this invention, and/or tubulin and/or microtubules and/or other moieties respond to applied electromagnetic fields.

As is disclosed at column 1 of U.S. Pat. No. 4,361,153 (see lines 9 et seq.), “Externally applied oscillating magnetic fields have been used before with implanted devices. Early inductive cardiac pacers employed externally generated electromagnetic energy directly as a power source. A coil inside the implant operated as a secondary transformer winding and was interconnected with the stimulating electrodes. More recently, implanted stimulators with rechargeable (e.g., nickel cadmium) batteries have used magnetic transmission to couple energy into a secondary winding in the implant to energize a recharging circuit having suitable rectifier circuitry. Miniature reed switches have been utilized before for implant communications. They appear to have been first used to allow the patient to convert from standby or demand mode to fixed rate pacing with an external magnet. Later, with the advent of programmable stimulators, reed switches were rapidly cycled by magnetic pulse transmission to operate pulse parameter selection circuitry inside the implant. Systems analogous to conventional two-way radio frequency (RF) and optical communication system have also been proposed. The increasing versatility of implanted stimulators demands more complex programming capabilities. While various systems for transmitting data into the implant have been proposed, there is a parallel need to develop compatible telemetry systems for signalling out of the implant. However, the austere energy budget constraints imposed by long life, battery operated implants rule out conventional transmitters and analogous systems”.

The solution provided by U.S. Pat. No. 4,361,153 is “ . . . achieved by the use of a resonant impedance modulated transponder in the implant to modulate the phase of a relatively high energy reflected magnetic carrier imposed from outside of the body.” In particular, and as is described by claim 1 of this patent, there is claimed “An apparatus for communicating variable information to an external device from an electronic stimulator implanted in a living human patient, comprising an external unit including means for transmitting a carrier signal, a hermetically sealed fully implantable enclosure adapted to be implanted at a fixed location in the patient's body, means within said enclosure for generating stimulator outputs, a transponder within said enclosure including tuned resonant circuit means for resonating at the frequency of said carrier signal so as to re-radiate a signal at the frequency of said carrier signal, and means for superimposing an information signal on the reflected signal by altering the resonance of said tuned circuit means in accordance with an information signal, said superimposing means including a variable impedance load connected across said tuned circuit and means for varying the impedance of said load in accordance with an information signal, said external unit further including pickup means for receiving the reflected signal from said transponder and means for recovering the information signal superimposed thereon, said receiving means including means reponsive to said reflected signal from said transponder for producing on associated analog output signal, and said recovering means including phase shift detector means responsive to said analog output signal for producing an output signal related to the relative phase angle thereof.”

U.S. Pat. No. 4,408,607 discloses a rechargeable, implantable capacitive energy source; the entire disclosure of this patent is hereby incorporated into this specification by reference; and this source may be used to directly or indirectly supply energy to one or more of the anti-mitotic compounds of this invention, and/or tubulin and/or microtubules and/or other moieties. As is disclosed in column 1 of such patent (at lines 12 et seq.), “Medical science has advanced to the point where it is possible to implant directly within living bodies electrical devices necessary or advantageous to the welfare of individual patients. A problem with such devices is how to supply the electrical energy necessary for their continued operation. The devices are, of course, designed to require a minimum of electrical energy, so that extended operation from batteries may be possible. Lithium batteries and other primary, non-rechargeable cells may be used, but they are expensive and require replacement of surgical procedures. Nickel-cadmium and other rechargeable batteries are also available, but have limited charge-recharge characteristics, require long intervals for recharging, and release gas during the charging process.”

The solution to this problem is described, e.g., in claim 1 of the patent, which describes “An electric power supply for providing electrical energy to an electrically operated medical device comprising: capacitor means for accommodating an electric charge; first means providing a regulated source of unidirectional electrical energy; second means connecting said first means to said capacitor means for supplying charging current to said capacitor means at a first voltage which increases with charge in the capacitor means; third means deriving from said first means a comparison second voltage of constant magnitude; comparator means operative when said first voltage reaches a first value to reduce said first voltage to a second, lower value; and voltage regulator means connected to said capacitor means and medical device to limit the voltage supplied to the medical device.”

U.S. Pat. No. 4,416,283 discloses a implantable shunted coil telemetry transponder employed as a magnetic pulse transducer for receiving externally transmitted data; the entire disclosure of this United States patent is hereby incorporated by reference into this specification. This transponder may be used in a manner similar to that of the aforementioned telemetry system.

In particular, a programming system for a biomedical implant is described in claim 1 of U.S. Pat. No. 4,416,283. Such claim 1 discloses “In a programming system for a biomedical implant of the type wherein an external programmer produces a series of magnetic impulses which are received and transduced to form a corresponding electrical pulse input to programmable parameter data registers inside the implant, wherein the improvement comprises external programming pulse receiving and transducing circuitry in the implant including a tuned coil, means responsive to pairs of successive voltage spikes of opposite polarity magnetically induced across said tuned coil by said magnetic impulses for forming corresponding binary pulses duplicating said externally generated magnetic impulses giving rise to said spikes, and means for outputting said binary pulses to said data registers to accomplish programming of the implant.”

U.S. Pat. No. 4,871,351 discloses an implantable pump infusion system; the entire disclosure of this United States patent is hereby incorporated by reference into this specification. These implantable pumps are discussed in column 1 of the patent, wherein it is disclosed that: “Certain human disorders, such as diabetes, require the injection into the body of prescribed amounts of medication at prescribed times or in response to particular conditions or events. Various kinds of infusion pumps have been propounded for infusing drugs or other chemicals or solutions into the body at continuous rates or measured dosages. Examples of such known infusion pumps and dispensing devices are found in U.S. Pat. Nos. 3,731,861; 3,692,027; 3,923,060; 4,003,379; 3,951,147; 4,193,397; 4,221,219 and 4,258,711. Some of the known pumps are external and inject the drugs or other medication into the body via a catheter, but the preferred pumps are those which are fully implantable in the human body.” One may use the implantable pumps of this patent to delivery the anti-mitotic compound of this invention to a specified site and, thereafter, to “finely focus” such delivery by means of magnetic focusing means.

U.S. Pat. No. 4,871,351 also discloses that: “Implantable pumps have been used in infusion systems such as those disclosed in U.S. Pat. Nos. 4,077,405; 4,282,872; 4,270,532; 4,360,019 and 4,373,527. Such infusion systems are of the open loop type. That is, the systems are pre-programmed to deliver a desired rate of infusion. The rate of infusion may be programmed to vary with time and the particular patient. A major disadvantage of such open loop systems is that they are not responsive to the current condition of the patient, i.e. they do not have feedback information. Thus, an infusion system of the open loop type may continue dispensing medication according to its pre-programmed rate or profile when, in fact, it may not be needed.”

U.S. Pat. No. 4,871,351 also discloses that: “There are known closed loop infusion systems which are designed to control a particular condition of the body, e.g. the blood glucose concentration. Such systems use feedback control continuously, i.e. the patient's blood is withdrawn via an intravenous catheter and analysed continuously and a computer output signal is derived from the actual blood glucose concentration to drive a pump which infuses insulin at a rate corresponding to the signal. The known closed loop systems suffer from several disadvantages. First, since they monitor the blood glucose concentration continuously they are complex and relatively bulky systems external to the patient, and restrict the movement of the patient. Such systems are suitable only for hospital bedside applications for short periods of time and require highly trained operating staff. Further, some of the known closed loop systems do not allow for manually input overriding commands. Examples of closed loop systems are found in U.S. Pat. Nos. 4,055,175; 4,151,845 and 4,245,634.”

U.S. Pat. No. 4,871,351 also discloses that “An implanted closed loop system with some degree of external control is disclosed in U.S. Pat. No. 4,146,029. In that system, a sensor (either implanted or external) is arranged on the body to sense some kind of physiological, chemical, electrical or other condition at a particular site and produced data which corresponds to the sensed condition at the sensed site. This data is fed directly to an implanted microprocessor controlled medication dispensing device. A predetermined amount of medication is dispensed in response to the sensed condition according to a pre-programmed algorithm in the microprocessor control unit. An extra-corporeal coding pulse transmitter is provided for selecting between different algorithms in the microprocessor control unit. The system of U.S. Pat. No. 4,146,029 is suitable for use in treating only certain ailments such as cardiac conditions. It is unsuitable as a blood glucose control system for example, since (i) it is not practicable to measure the blood glucose concentration continuously with an implanted sensor and (ii) the known system is incapable of dispensing discrete doses of insulin in response to certain events, such as meals and exercise. Furthermore, there are several disadvantages to internal sensors; namely, due to drift, lack of regular calibration and limited life, internal sensors do not have high long-term reliability. If an external sensor is used with the system of U.S. Pat. No. 4,146,029, the output of the sensor must be fed through the patient's skin to the implanted mechanism. There are inherent disadvantages to such a system, namely the high risk of infection. Since the algorithms which control the rate of infusion are programmed into the implanted unit, it is not possible to upgrade these algorithms without surgery. The extra-corporeal controller merely selects a particular one of several medication programs but cannot actually alter a program.”

U.S. Pat. No. 4,871,351 also discloses that “It is an object of the present invention to overcome, or substantially ameliorate the above described disadvantages of the prior art by providing an implantable open loop medication infusion system with a feedback control option.”

The solution to this problem is set forth in claim 1 of U.S. Pat. No. 4,871,351, which describes: “A medical infusion system intermittently switchable at selected times between an open loop system without feedback and a closed loop system with feedback, said system comprising an implantable unit including means for controllably dispensing medication into a body, an external controller, and an extra-corporeal sensor; wherein said implantable unit comprises an implantable transceiver means for communicating with a similar external transceiver means in said external controller to provide a telemetry link between said controller and said implantable unit, a first reservoir means for holding medication liquid, a liquid dispensing device, a pump connected between said reservoir means and said liquid dispensing device, and a first electronic control circuit means connected to said implantable transceiver means and to said pump to operate said pump; wherein said external controller comprises a second electronic control circuit means connected with said external transceiver means, a transducer means for reading said sensor, said transducer means having an output connected to said second electronic control circuit means, and a manually operable electric input device connected to said second electronic control circuit means; wherein said pump is operable by said first electronic control circuit means to pump said medication liquid from said first reservoir means to said liquid-dispensing deive at a first predetermined rate independent of the output of said extra-corporeal sensor, and wherein said input device or said transducer means include means which selectively operable at intermittent times to respectively convey commands or output of said transducer representing the reading of said sensor to said second control circuit to instruct said first control circuit via said telemetry link to modify the operation of said pump.” U.S. Pat. No. 4,941,461 describes an electrically actuated inflatable penile erecton device comprised of an implantable induction coil and an implantable pump; the entire disclosure of this United States patent is hereby incorporated by reference into this specification. The device of this patent is described, e.g., in claim 1 of the patent, which discloses “An apparatus for achieving a penile erection in a human male, comprising: at least one elastomer cylinder having a root chamber and a pendulous chamber, said elastomer cylinder adapted to be placed in the corpus carvenosum of the penis; an external magnetic field generator which can be placed over some section of the penis which generates an alternating magnetic field; an induction coil contained within said elastomer cylinder which produces an alternating electric current when in the proximity of said alternating magnetic filed which is produced by said external magnetic field generator; and a fluid pumping means located within said elastomer cylinder, said pumping means being operated by the electrical power generated in said induction coil to pump fluid from said root chamber to said pendulous chamber in order to stiffen said elastomer cylinder for causing the erect state of the penis.”

U.S. Pat. No. 5,487,760 discloses an implantable signal transceiver disposed in an artificial heart valve; this transceiver may be used in the process of this invention in accordance with the aforementioned telemetry device; and the entire disclosure of this United States patent is hereby incorporated by reference into this specification. Claim 1 of this patent describes: “In combination, an artificial heart valve of the type having a tubular body member, defining a lumen and pivotally supporting at least one occluder, said body member having a sewing cuff covering an exterior surface of said body member; and an electronic sensor module disposed between said sewing cuff and said exterior surface, wherein said sensor module incorporates a sensor element for detecting movement of said at least one occluder between an open and a closed disposition relative to said lumen and wherein said sensor module further includes a signal transceiver coupled to said sensor element, and means for energizing said signal transceiver, and wherein said sensor module includes means for encapsulating said sensor element, signal transceiver and energizing means in a moisture-impervious container.” As will be apparent to those skilled in the art, the sensor/transceiver combination may advantageously be used in conjunction with the anti-mitotic compound of this invention, and/or microtubules.

U.S. Pat. No. 5,702,430 discloses an implantable power supply; the entire disclosure of such patent is hereby incorporated by reference into this specification. This implantable power supply may be used to supply power to either the compound of this invention, the treatment site, and/or one or more other devices from which a specified energy output is desired.

Claim 1 of U.S. Pat. No. 5,702,430 describes: “A surgically implantable power supply comprising battery means for providing a source of power, charging means for charging the battery means, enclosure means isolating the battery means from the human body, gas holding means within the enclosure means for holding gas generated by the battery means during charging, seal means in the enclosure means arranged to rapture when the internal gas pressure exceeds a certain value and inflatable gas container means outside the enclosure means to receive gas from within the enclosure means when the seal means has been ruptured.”

Columns 1 through 5 of U.S. Pat. No. 5,702,430 presents an excellent discussion of “prior art” implantable pump assemblies that may be used, e.g., to deliver the anti-mitotic compound of this invention. As is disclosed in such portion of U.S. Pat. No. 5,702,430, “The most widely tested and commonly used implantable blood pumps employ variable forms of flexible sacks (also spelled sacs) or diaphragms which are squeezed and released in a cyclical manner to cause pulsatile ejection of blood. Such pumps are discussed in books or articles such as Hogness and Antwerp 1991, DeVries et al 1984, and Farrar et al 1988, and in U.S. Pat. No. 4,994,078 (Jarvik 1991), U.S. Pat. No. 4,704,120 (Slonina 1987), U.S. Pat. No. 4,936,758 (Coble 1990), and U.S. Pat. No. 4,969,864 (Schwarzmann et al 1990). Sack or diaphragm pumps are subject to fatigue failure of compliant elements and as such are mechanically and functionally quite different from the pump which is the subject of the present invention.”

U.S. Pat. No. 5,702,430 also discloses that “An entirely different class of implantable blood pumps uses rotary pumping mechanisms. Most rotary pumps can be classified into two categories: centrifugal pumps and axial pumps. Centrifugal pumps, which include pumps marketed by Sarns (a subsidiary of the 3M Company) and Biomedicus (a subsidiary of Medtronic, Eden Prairie, Minn.), direct blood into a chamber, against a spinning interior wall (which is a smooth disk in the Medtronic pump). A flow channel is provided so that the centrifugal force exerted on the blood generates flow.”

U.S. Pat. No. 5,702,430 also discloses that “By contrast, axial pumps provide blood flow along a cylindrical axis, which is in a straight (or nearly straight) line with the direction of the inflow and outflow. Depending on the pumping mechanism used inside an axial pump, this can in some cases reduce the shearing effects of the rapid acceleration and deceleration forces generated in centrifugal pumps. However, the mechanisms used by axial pumps can inflict other types of stress and damage on blood cells.”

U.S. Pat. No. 5,702,430 also discloses that “Some types of axial rotary pumps use impeller blades mounted on a center axle, which is mounted inside a tubular conduit. As the blade assembly spins, it functions like a fan, or an outboard motor propeller. As used herein, “impeller” refers to angled vanes (also called blades) which are constrained inside a flow conduit; an impeller imparts force to a fluid that flows through the conduit which encloses the impeller. By contrast, “propeller” usually refers to non-enclosed devices, which typically are used to propel vehicles such as boats or airplanes.”

“Another type of axial blood pump, called the “Haemopump” (sold by Nimbus) uses a screw-type impeller with a classic screw (also called an Archimedes screw; also called a helifoil, due to its helical shape and thin cross-section). Instead of using several relatively small vanes, the Haemopump screw-type impeller contains a single elongated helix, comparable to an auger used for drilling or digging holes. In screw-type axial pumps, the screw spins at very high speed (up to about 10,000 rpm). The entire Haemopump unit is usually less than a centimeter in diameter. The pump can be passed through a peripheral artery into the aorta, through the aortic valve, and into the left ventricle. It is powered by an external motor and drive unit.”

U.S. Pat. No. 5,702,430 also discloses that “Centrifugal or axial pumps are commonly used in three situations: (1) for brief support during cardio-pulmonary operations, (2) for short-term support while awaiting recovery of the heart from surgery, or (3) as a bridge to keep a patient alive while awaiting heart transplantation. However, rotary pumps generally are not well tolerated for any prolonged period. Patients who must rely on these units for a substantial length of time often suffer from strokes, renal (kidney) failure, and other organ dysfunction. This is due to the fact that rotary devices, which must operate at relatively high speeds, may impose unacceptably high levels of turbulent and laminar shear forces on blood cells. These forces can damage or lyse (break apart) red blood cells. A low blood count (anemia) may result, and the disgorged contents of lysed blood cells (which include large quantities of hemoglobin) can cause renal failure and lead to platelet activation that can cause embolisms and stroke.”

“One of the most important problems in axial rotary pumps in the prior art involves the gaps that exist between the outer edges of the blades, and the walls of the flow conduit. These gaps are the site of severe turbulence and shear stresses, due to two factors. Since implantable axial pumps operate at very high speed, the outer edges of the blades move extremely fast and generate high levels of shear and turbulence. In addition, the gap between the blades and the wall is usually kept as small as possible to increase pumping efficiency and to reduce the number of cells that become entrained in the gap area. This can lead to high-speed compression of blood cells as they are caught in a narrow gap between the stationary interior wall of the conduit and the rapidly moving tips or edges of the blades.”

U.S. Pat. No. 5,702,430 also discloses that “An important factor that needs to be considered in the design and use of implantable blood pumps is “residual cardiac function,” which is present in the overwhelming majority of patients who would be candidates for mechanical circulatory assistance. The patient's heart is still present and still beating, even though, in patients who need mechanical pumping assistance, its output is not adequate for the patient's needs. In many patients, residual cardiac functioning often approaches the level of adequacy required to support the body, as evidenced by the fact that the patient is still alive when implantation of an artificial pump must be considered and decided. If cardiac function drops to a level of severe inadequacy, death quickly becomes imminent, and the need for immediate intervention to avert death becomes acute.’”

U.S. Pat. No. 5,702,430 also discloses that “Most conventional ventricular assist devices are designed to assume complete circulatory responsibilities for the ventricle they are “assisting. As such, there is no need, nor presumably any advantage, for the device to interact in harmony with the assisted ventricle. Typically, these devices utilize a “fill-to-empty” mode that, for the most part, results in emptying of the device in random association with native heart contraction. This type of interaction between the device and assisted ventricle ignores the fact that the overwhelming majority of patients who would be candidates for mechanical assistance have at least some significant residual cardiac function.”

U.S. Pat. No. 5,702,430 also discloses that “It is preferable to allow the natural heart, no matter how badly damaged or diseased it may be, to continue contributing to the required cardiac output whenever possible so that ventricular hemodynamics are disturbed as little as possible. This points away from the use of total cardiac replacements and suggests the use of “assist” devices whenever possible. However, the use of assist devices also poses a very difficult problem: in patients suffering from severe heart disease, temporary or intermittent crises often require artificial pumps to provide “bridging” support which is sufficient to entirely replace ventricular pumping capacity for limited periods of time, such as in the hours or days following a heart attack or cardiac arrest, or during periods of severe tachycardia or fibrillation.”

U.S. Pat. No. 5,702,430 also discloses that “Accordingly, an important goal during development of the described method of pump implantation and use and of the surgically implantable reciprocating pump was to design a method and a device which could cover a wide spectrum of requirements by providing two different and distinct functions. First, an ideal cardiac pumping device should be able to provide “total” or “complete” pumping support which can keep the patient alive for brief or even prolonged periods, if the patient's heart suffers from a period of total failure or severe inadequacy. Second, in addition to being able to provide total pumping support for the body during brief periods, the pump should also be able to provide a limited “assist” function. It should be able to interact with a beating heart in a cooperative manner, with minimal disruption of the blood flow generated by the natural heartbeat. If a ventricle is still functional and able to contribute to cardiac output, as is the case in the overwhelming majority of clinical applications, then the pump will assist or augment the residual cardiac output. This allows it to take advantage of the natural, non-hemolytic pumping action of the heart to the fullest extent possible; it minimizes red blood cell lysis, it reduces mechanical stress on the pump, and it allows longer pump life and longer battery life.”

“Several types of surgically implantable blood pumps containing a piston-like member have been developed to provide a mechanical device for augmenting or even totally replacing the blood pumping action of a damaged or diseased mammalian heart.”

“U.S. Pat. No. 3,842,440 to Karlson discloses an implantable linear motor prosthetic heart and control system containing a pump having a piston-like member which is reciprocal within a magnetic field. The piston-like member includes a compressible chamber in the prosthetic heart which communicates with the vein or aorta.”

U.S. Pat. No. 5,702,430 also discloses that “U.S. Pat. Nos. 3,911,897 and 3,911,898 to Leachman, Jr. disclose heart assist devices controlled in the normal mode of operation to copulsate and counterpulsate with the heart, respectively, and produce a blood flow waveform corresponding to the blood flow waveform of the heart being assisted. The heart assist device is a pump connected serially between the discharge of a heart ventricle and the vascular system. The pump may be connected to the aorta between the left ventricle discharge immediately adjacent the aortic valve and a ligation in the aorta a short distance from the discharge. This pump has coaxially aligned cylindrical inlet and discharge pumping chambers of the same diameter and a reciprocating piston in one chamber fixedly connected with a reciprocating piston of the other chamber. The piston pump further includes a passageway leading between the inlet and discharge chambers and a check valve in the passageway preventing flow from the discharge chamber into the inlet chamber. There is no flow through the movable element of the piston.”

U.S. Pat. No. 5,702,430 also discloses that “U.S. Pat. No. 4,102,610 to Taboada et al. discloses a magnetically operated constant volume reciprocating pump which can be used as a surgically implantable heart pump or assist. The reciprocating member is a piston carrying a tilting-disk type check valve positioned in a cylinder. While a tilting disk valve results in less turbulence and applied shear to surrounding fluid than a squeezed flexible sack or rotating impeller, the shear applied may still be sufficiently excessive so as to cause damage to red blood cells.”

U.S. Pat. No. 5,702,430 also discloses that “U.S. Pat. Nos. 4,210,409 and 4,375,941 to Child disclose a pump used to assist pumping action of the heart having a piston movable in a cylindrical casing in response to magnetic forces. A tilting-disk type check valve carried by the piston provides for flow of fluid into the cylindrical casing and restricts reverse flow. A plurality of longitudinal vanes integral with the inner wall of the cylindrical casing allow for limited reverse movement of blood around the piston which may result in compression and additional shearing of red blood cells. A second fixed valve is present in the inlet of the valve to prevent reversal of flow during piston reversal.”

U.S. Pat. No. 5,702,430 also discloses that “U.S. Pat. No. 4,965,864 to Roth discloses a linear motor using multiple coils and a reciprocating element containing permanent magnets which is driven by microprocessor-controlled power semiconductors. A plurality of permanent magnets is mounted on the reciprocating member. This design does not provide for self-synchronization of the linear motor in the event the stroke of the linear motor is greater than twice the pole pitch on the reciprocating element. During start-up of the motor, or if magnetic coupling is lost, the reciprocating element may slip from its synchronous position by any multiple of two times the pole pitch. As a result, a sensing arrangement must be included in the design to detect the position of the piston so that the controller will not drive it into one end of the closed cylinder. In addition, this design having equal pole pitch and slot pitch results in a “jumpy” motion of the reciprocating element along its stroke.”

U.S. Pat. No. 5,702,430 also discloses that “In addition to the piston position sensing arrangement discussed above, the Roth design may also include a temperature sensor and a pressure sensor as well as control circuitry responsive to the sensors to produce the intended piston motion. For applications such as implantable blood pumps where replacement of failed or malfunctioning sensors requires open heart surgery, it is unacceptable to have a linear motor drive and controller that relies on any such sensors. In addition, the Roth controller circuit uses only NPN transistors thereby restricting current flow to the motor windings to one direction only.’

‘U.S. Pat. No. 4,541,787 to Delong describes a pump configuration wherein a piston containing a permanent magnet is driven in a reciprocating fashion along the length of a cylinder by energizing a sequence of coils positioned around the outside of the cylinder. However, the coil and control system configurations disclosed only allow current to flow through one individual winding at a time. This does not make effective use of the magnetic flux produced by each pole of the magnet in the piston. To maximize force applied to the piston in a given direction, current must flow in one direction in the coils surrounding the vicinity of the north pole of the permanent magnet while current flows in the opposite direction in the coils surrounding the vicinity of the south pole of the permanent magnet. Further, during starting of the pump disclosed by Delong, if the magnetic piston is not in the vicinity of the first coil energized, the sequence of coils that are subsequently energized will ultimately approach and repel the magnetic piston toward one end of the closed cylinder. Consequently, the piston must be driven into the end of the closed cylinder before the magnetic poles created by the external coils can become coupled with the poles of the magnetic piston in attraction.”

U.S. Pat. No. 5,702,430 also discloses that “U.S. Pat. No. 4,610,658 to Buchwald et al. discloses an implantable fluid displacement peritoneovenous shunt system. The system comprises a magnetically driven pump having a spool piston fitted with a disc flap valve.”

U.S. Pat. No. 5,702,430 also discloses that “U.S. Pat. No. 5,089,017 to Young et al. discloses a drive system for artificial hearts and left ventricular assist devices comprising one or more implantable pumps driven by external electromagnets. The pump utilizes working fluid, such as sulfur hexafluoride to apply pneumatic pressure to increase blood pressure and flow rate.”

U.S. Pat. No. 5,743,854 discloses a device for inducing and localizing epileptiform activity that is comprised of a direct current (DC) magnetic field generator, a DC power source, and sensors adapted to be coupled to a patient's head; this direct current magnetic field generator may be used in conjunction with the anti-mitotic compound of this invention and/or an auxiliary device and/or tubulin and/or microtubules. In one embodiment of the invention, described in claim 7, the sensors “ . . . comprise Foramen Ovale electrodes adapted to be implanted to sense evoked and natural epileptic firings.”

U.S. Pat. No. 5,803,897 discloses a penile prosthesis system comprised of an implantable pressurized chamber, a reservoir, a rotary pump, a magnetically responsive rotor, and a rotary magnetic field generator. Claim 1 of this patent describes: “A penile prosthesis system comprising: at least one pressurizable chamber including a fluid port, said chamber adapted to be located within the penis of a patient for tending to make the penis rigid in response to fluid pressure within said chamber; a fluid reservoir; a rotary pump adapted to be implanted within the body of a user, said rotary pump being coupled to said reservoir and to said chamber, said rotary pump including a magnetically responsive rotor adapted for rotation in the presence of a rotating magnetic field, and an impeller for tending to pump fluid at least from said reservoir to said chamber under the impetus of fluid pressure, to thereby pressurize said chamber in response to operation of said pump; and a rotary magnetic field generator for generating a rotating magnetic field, for, when placed adjacent to the skin of said user at a location near said rotary pump, rotating said magnetically responsive rotor in response to said rotating magnetic field, to thereby tend to pressurize said chamber and to render the penis rigid; controllable valve means operable in response to motion of said rotor of said rotary pump, for tending to prevent depressurization of said chamber when said rotating magnetic field no longer acts on said rotor, said controllable valve means comprising a unidirectional check valve located in the fluid path extending between said rotary pump and said port of said chamber.” Such fluid pumping means may be used to facilitate the delivery of the anti-mitotic compound of this invention.

U.S. Pat. No. 5,810,015 describes an implantable power supply that can convert non-electrical energy (such as mechanical, chemical, thermal, or nuclear energy) into electrical energy; the entire disclosure of this United States patent is hereby incorporated by reference into this specification. This power supply may be used to supply energy to the anti-mitotic compound of this invention and/or to tubulin and/or to microtubules.

In column 1 of U.S. Pat. No. 5,810,015, a discussion of “prior art” rechargeable power supplies is presented. It is disclosed in this column 1 that: “Modern medical science employs numerous electrically powered devices which are implanted in a living body. For example, such devices may be employed to deliver medications, to support blood circulation as in a cardiac pacemaker or artificial heart, and the like. Many implantable devices contain batteries which may be rechargeable by transcutaneous induction of electromagnetic fields in implanted coils connected to the batteries. Transcutaneous inductive recharging of batteries in implanted devices is disclosed for example in U.S. Pat. Nos. 3,923,060; 4,082,097; 4,143,661; 4,665,896; 5,279,292; 5,314,453; 5,372,605, and many others.”

U.S. Pat. No. 5,810,015 also discloses that: “Other methods for recharging implanted batteries have also been attempted. For example, U.S. Pat. No. 4,432,363 discloses use of light or heat to power a solar battery within an implanted device. U.S. Pat. No. 4,661,107 discloses recharging of a pacemaker battery using mechanical energy created by motion of an implanted heart valve.” These “other methods” may also be used in the process of this invention.

U.S. Pat. No. 5,810,015 also discloses that: “A number of implanted devices have been powered without batteries. U.S. Pat. Nos. 3,486,506 and 3,554,199 disclose generation of electric pulses in an implanted device by movement of a rotor in response to the patient's heartbeat. U.S. Pat. No. 3,563,245 discloses a miniaturized power supply unit which employs mechanical energy of heart muscle contractions to generate electrical energy for a pacemaker. U.S. Pat. No. 3,456,134 discloses a piezoelectric converter for electronic implants in which a piezoelectric crystal is in the form of a weighted cantilever beam capable of responding to body movement to generate electric pulses. U.S. Pat. No. 3,659,615 also discloses a piezoelectric converter which reacts to muscular movement in the area of implantation. U.S. Pat. No. 4,453,537 discloses a pressure actuated artificial heart powered by a second implanted device attached to a body muscle which in turn is stimulated by an electric signal generated by a pacemaker.” These “other devices” may also be used in the process of this invention.

U.S. Pat. No. 5,810,015 also discloses that: “In spite of all these efforts, a need remains for efficient generation of energy to supply electrically powered implanted devices.” The solution provided by U.S. Pat. No. 5,80,015 is described in claim 1 thereof, which describes: “An implantable power supply apparatus for supplying electrical energy to an electrically powered device, comprising: a power supply unit including: a transcutaneously, invasively rechargeable non-electrical energy storage device (NESD); an electrical energy storage device (EESD); and an energy converter coupling said NESD and said EESD, said converter including means for converting non-electrical energy stored in said NESD to electrical energy and for transferring said electrical energy to said EESD, thereby storing said electrical energy in said EESD.”

An implantable ultrasound communicaton system is disclosed in U.S. Pat. No. 5,861,018, the entire disclosure of which is hereby incorporated by reference into this specification. As is disclosed in the abstract of this patent, there is disclosed in such patent “A system for communicating through the skin of a patient, the system including an internal communication device implanted inside the body of a patient and an external communication device. The external communication device includes an external transmitter which transmits a carrier signal into the body of the patient during communication from the internal communication device to the external communication device. The internal communication device includes an internal modulator which modulates the carrier signal with information by selectively reflecting the carrier signal or not reflecting the carrier signal. The external communication device demodulates the carrier signal by detecting when the carrier signal is reflected and when the carrier signal is not reflected through the skin of the patient. When the reflected carrier signal is detected, it is interpreted as data of a first state, and when the reelected carrier signal is not detected, it is interpreted as data of a second state. Accordingly, the internal communication device consumes relatively little power because the carrier signal used to carry the information is derived from the external communication device. Further, transfer of data is also very efficient because the period needed to modulate information of either the first state or the second state onto the carrier signal is the same. In one embodiment, the carrier signal operates in the ultrasound frequency range.”

U.S. Pat. No. 5,861,019, the entire disclosure of which is hereby incorporated by reference into this specification, discloses a telemetry system for communications between an external programmer and an implantable medical device. Claim 1 of this patent describes: “A telemetry system for communications between an external programmer and an implantable medical device, comprising: the external programmer comprising an external telemetry antenna and an external transceiver for receiving uplink telemetry transmissions and transmitting downlink telemetry transmission through the external telemetry antenna; the implantable medical device comprising an implantable medical device housing, an implantable telemetry antenna and an implantable transceiver for receiving downlink transmissions and for transmitting uplink telemetry transmission through the implantable telemetry antenna, the implantable medical device housing being formed of a conductive metal and having an exterior housing surface and an interior housing surface; the implantable medical device housing being formed with a housing recess extending inwardly from the exterior housing surface to a predetermined housing recess depth in the predetermined substrate area of the exterior housing surface for receiving the dielectric substrate therein; wherein the implantable telemetry antenna is a conformal microstrip antenna formed as part of the implantable medical device housing, the microstrip antenna having electrically conductive ground plane and radiator patch layers separated by a dielectric substrate, layer the conductive radiator patch layer having a predetermined thickness and predetermined radiator patch layer dimensions, the patch layer being formed upon one side of the dielectric substrate layer.”

“An extensive description of the historical development of uplink and downlink telemetry transmission formats” is set forth at columns 2 through 5 of U.S. Pat. No. 5,861,019; such telemetry transmission formats may be used in conjunction with the anti-mitotic compound of this invention. As is disclosed in these columns: “An extensive description of the historical development of uplink and downlink telemetry transmission formats and is set forth in the above-referenced '851 and '963 applications and in the following series of commonly assigned patents all of which are incorporated herein by reference in their entireties. Commonly assigned U.S. Pat. No. 5,127,404 to Grevious et al. sets forth an improved method of frame based, pulse position modulated (PPM) of data particularly for uplink telemetry. The frame-based PPM telemetry format increases bandwidth well above simple PIM or pulse width modulation (PWM) binary bit stream transmissions and thereby conserves energy of the implanted medical device. Commonly assigned U.S. Pat. No. 5,168,871 to Grevious et al. sets forth an improvement in the telemetry system of the '404 patent for detecting uplink telemetry RF pulse bursts that are corrupted in a noisy environment. Commonly assigned U.S. Pat. No. 5,292,343 to Blanchette et al. sets forth a further improvement in the telemetry system of the '404 patent employing a hand shake protocol for maintaining the communications link between the external programmer and the implanted medical device despite instability in holding the programmer RF head steady during the transmission. Commonly assigned U.S. Pat. No. 5,324,315 to Grevious sets forth an improvement in the uplink telemetry system of the '404 patent for providing feedback to the programmer to aid in optimally positioning the programmer RF head over the implanted medical device. Commonly assigned U.S. Pat. No. 5,117,825 to Grevious sets forth an further improvement in the programmer RF head for regulating the output level of the magnetic H field of the RF head telemetry antenna using a signal induced in a sense coil in a feedback loop to control gain of an amplifier driving the RF head telemetry antenna. Commonly assigned U.S. Pat. No. 5,562,714 to Grevious sets forth a further solution to the regulation of the output level of the magnetic H field generated by the RF head telemetry antenna using the sense coil current to directly load the H field. Commonly assigned U.S. Pat. No. 5,354,319 to Wybomey et al. sets forth a number of further improvements in the frame based telemetry system of the '404 patent. Many of these improvements are incorporated into MEDTRONIC® Model 9760, 9766 and 9790 programmers. These improvements and the improvements described in the above-referenced pending patent applications are directed in general to increasing the data transmission rate, decreasing current consumption of the battery power source of the implantable medical device, and increasing reliability of uplink and downlink telemetry transmissions.”

U.S. Pat. No. 5,810,015 also discloses that: “The current MEDTRONIC® telemetry system employing the 175 kHz carrier frequency limits the upper data transfer rate, depending on bandwidth and the prevailing signal-to-noise ratio. Using a ferrite core, wire coil, RF telemetry antenna results in: (1) a very low radiation efficiency because of feed impedance mismatch and ohmic losses; 2) a radiation intensity attenuated proportionally to at least the fourth power of distance (in contrast to other radiation systems which have radiation intensity attenuated proportionally to square of distance); and 3) good noise immunity because of the required close distance between and coupling of the receiver and transmitter RF telemetry antenna fields.”

U.S. Pat. No. 5,810,015 also discloses that “These characteristics require that the implantable medical device be implanted just under the patient's skin and preferably oriented with the RF telemetry antenna closest to the patient's skin. To ensure that the data transfer is reliable, it is necessary for the patient to remain still and for the medical professional to steadily hold the RF programmer head against the patient's skin over the implanted medical device for the duration of the transmission. If the telemetry transmission takes a relatively long number of seconds, there is a chance that the programmer head will not be held steady. If the uplink telemetry transmission link is interrupted by a gross movement, it is necessary to restart and repeat the uplink telemetry transmission. Many of the above-incorporated, commonly assigned, patents address these problems.”

U.S. Pat. No. 5,810,015 also discloses that “The ferrite core, wire coil, RF telemetry antenna is not bio-compatible, and therefore it must be placed inside the medical device hermetically sealed housing. The typically conductive medical device housing adversely attenuates the radiated RF field and limits the data transfer distance between the programmer head and the implanted medical device RF telemetry antennas to a few inches.”

U.S. Pat. No. 5,810,015 also discloses that “In U.S. Pat. No. 4,785,827 to Fischer, U.S. Pat. No. 4,991,582 to Byers et al., and commonly assigned U.S. Pat. No. 5,470,345 to Hassler et al. (all incorporated herein by reference in their entireties), the metal can typically used as the hermetically sealed housing of the implantable medical device is replaced by a hermetically sealed ceramic container. The wire coil antenna is still placed inside the container, but the magnetic H field is less attenuated. It is still necessary to maintain the implanted medical device and the external programming head in relatively close proximity to ensure that the H field coupling is maintained between the respective RF telemetry antennas.”

U.S. Pat. No. 5,810,015 also discloses that: “Attempts have been made to replace the ferrite core, wire coil, RF telemetry antenna in the implantable medical device with an antenna that can be located outside the hermetically sealed enclosure. For example, a relatively large air core RF telemetry antenna has been embedded into the thermoplastic header material of the MEDTRONIC® Prometheus programmable IPG. It is also suggested that the RF telemetry antenna may be located in the IPG header in U.S. Pat. No. 5,342,408. The header area and volume is relatively limited, and body fluid may infiltrate the header material and the RF telemetry antenna.”

U.S. Pat. No. 5,810,015 also discloses that: “In U.S. Pat. Nos. 5,058,581 and 5,562,713 to Silvian, incorporated herein by reference in their entireties, it is proposed that the elongated wire conductor of one or more medical lead extending away from the implanted medical device be employed as an RF telemetry antenna. In the particular examples, the medical lead is a cardiac lead particularly used to deliver energy to the heart generated by a pulse generator circuit and to conduct electrical heart signals to a sense amplifier. A modest increase in the data transmission rate to about 8 Kb/s is alleged in the '581 and '713 patents using an RF frequency of 10-300 MHz. In these cases, the conductor wire of the medical lead can operate as a far field radiator to a more remotely located programmer RF telemetry antenna. Consequently, it is not necessary to maintain a close spacing between the programmer RF telemetry antenna and the implanted cardiac lead antenna or for the patient to stay as still as possible during the telemetry transmission.”

U.S. Pat. No. 5,810,015 also discloses that: “However, using the medical lead conductor as the RF telemetry antenna has several disadvantages. The radiating field is maintained by current flowing in the lead conductor, and the use of the medical lead conductor during the RF telemetry transmission may conflict with sensing and stimulation operations. RF radiation losses are high because the human body medium is lossy at higher RF frequencies. The elongated lead wire RF telemetry antenna has directional radiation nulls that depend on the direction that the medical lead extends, which varies from patient to patient. These considerations both contribute to the requirement that uplink telemetry transmission energy be set artificially high to ensure that the radiated RF energy during the RF uplink telemetry can be detected at the programmer RF telemetry antenna. Moreover, not all implantable medical devices have lead conductor wires extending from the device.”

U.S. Pat. No. 5,810,015 also discloses that: “A further U.S. Pat. No. 4,681,111 to Silvian, incorporated herein by reference in its entirety, suggests the use of a stub antenna associated with the header as the implantable medical device RF telemetry antenna for high carrier frequencies of up to 200 MHz and employing phase shift keying (PSK) modulation. The elimination of the need for a VCO and a bit rate on the order of 2-5% of the carrier frequency or 3.3-10 times the conventional bit rate are alleged.”

U.S. Pat. No. 5,810,015 also discloses that: “At present, a wide variety of implanted medical devices are commercially released or proposed for clinical implantation. Such medical devices include implantable cardiac pacemakers as well as implantable cardioverter-defibrillators, pacemaker-cardioverter-defibrillators, drug delivery pumps, cardiomyostimulators, cardiac and other physiologic monitors, nerve and muscle stimulators, deep brain stimulators, cochlear implants, artificial hearts, etc. As the technology advances, implantable medical devices become ever more complex in possible programmable operating modes, menus of available operating parameters, and capabilities of monitoring increasing varieties of physiologic conditions and electrical signals which place ever increasing demands on the programming system.”

U.S. Pat. No. 5,810,015 also discloses that: “It remains desirable to minimize the time spent in uplink telemetry and downlink transmissions both to reduce the likelihood that the telemetry link may be broken and to reduce current consumption.”

“Moreover, it is desirable to eliminate the need to hold the programmer RF telemetry antenna still and in proximity with the implantable medical device RF telemetry antenna for the duration of the telemetry transmission. As will become apparent from the following, the present invention satisfies these needs.”

The solution to this problem is presented, e.g., in claim 1 of U.S. Pat. No. 5,861,019. This claim describes “A telemetry system for communications between an external programmer and an implantable medical device, comprising: the external programmer comprising an external telemetry antenna and an external transceiver for receiving uplink telemetry transmissions and transmitting downlink telemetry transmission through the external telemetry antenna; the implantable medical device comprising an implantable medical device housing, an implantable telemetry antenna and an implantable transceiver for receiving downlink transmissions and for transmitting uplink telemetry transmission through the implantable telemetry antenna, the implantable medical device housing being formed of a conductive metal and having an exterior housing surface and an interior housing surface; the implantable medical device housing being formed with a housing recess extending inwardly from the exterior housing surface to a predetermined housing recess depth in the predetermined substrate area of the exterior housing surface for receiving the dielectric substrate therein; wherein the implantable telemetry antenna is a conformal microstrip antenna formed as part of the implantable medical device housing, the microstrip antenna having electrically conductive ground plane and radiator patch layers separated by a dielectric substrate, layer the conductive radiator patch layer having a predetermined thickness and predetermined radiator patch layer dimensions, the patch layer being formed upon one side of the dielectric substrate layer.”

U.S. Pat. No. 5,945,762, the entire disclosure of which is hereby incorporated by reference into this specification, discloses an external transmitter adapted to magnetically excite an implanted receiver coil; such an implanted receiver coil may be disposed near, e.g., the anti-mitotic compound of this invention and/or other devices and/or tubulin and/or microtubules. Claim 1 of this patent describes “An external transmitter adapted for magnetically exciting an implanted receiver coil, causing an electrical current to flow in the implanted receiver coil, comprising: (a) a support; (b) a magnetic field generator that is mounted to the support; and (c) a prime mover that is drivingly coupled to an element of the magnetic field generator to cause said element of the magnetic field generator to reciprocate, in a reciprocal motion, said reciprocal motion of said element of the magnetic field generator producing a varying magnetic field that is adapted to induce an electrical current to flow in the implanted receiver coil.”

U.S. Pat. No. 5,954,758, the entire disclosure of which is hereby incorporated by reference into this specification, claims an implantable electrical stimulator comprised of an implantable radio frequency receiving coil, an implantable power supply, an implantable input signal generator, an implantable decoder, and an implantable electrical stimulator. Claim 1 of this patent describes “A system for transcutaneously telemetering position signals out of a human body and for controlling a functional electrical stimulator implanted in said human body, said system comprising: an implantable radio frequency receiving coil for receiving a transcutaneous radio frequency signal; an implantable power supply connected to said radio frequency receiving coil, said power supply converting received transcutaneous radio frequency signals into electromotive power; an implantable input signal generator electrically powered by said implantable power supply for generating at least one analog input movement signal to indicate voluntary bodily movement along an axis; an implantable encoder having an input operatively connected with said implantable input signal generator for encoding said movement signal into output data in a preselected data format; an impedance altering means connected with said encoder and said implantable radio frequency signal receiving coil to selectively change an impedance of said implantable radio frequency signal receiving coil; an external radio frequency signal transmit coil inductively coupled with said implantable radio frequency signal receiving coil, such that impedance changes in said implantable radio frequency signal receiving coil are sensed by said external radio frequency signal transmit coil to establish a sensed modulated movement signal in said external transmit coil; an external control system electrically connected to said external radio frequency transmit coil for monitoring said sensed modulated movement signal in said external radio frequency transmit coil, said external control system including: a demodulator for recovering the output data of said encoder from the sensed modulated ovement signal of said external transmit coil, a pulse width algorithm means for applying a preselected pulse width algorithm to the recovered output data to derive a first pulse width, an amplitude algorithm means for applying an amplitude algorithm to the recovered output data to derive a first amplitude therefrom, an interpulse interval algorithm means for applying an interpulse algorithm to the recovered output data to derive a first interpulse interval therefrom; and, a stimulation pulse train signal generator for generating a stimulus pulse train signal which has the first pulse width and the first pulse amplitude; an implantable functional electrical stimulator for receiving said stimulation pulse train signal from said stimulation pulse train signal generator and generating stimulation pulses with the first pulse width, the first pulse amplitude, and separated by the first interpulse interval; and, at least one electrode operatively connected with the functional electrical stimulator for applying said stimulation pulses to muscle tissue of said human body.”

U.S. Pat. No. 6,006,133, the entire disclosure of which is hereby incorporated by reference into this specification, describes an implantable medical device comprised of a hermetically sealed housing.” Such a hermetically sealed housing may be used to contain, e.g., the anti-mitotic compound of this invention.

U.S. Pat. No. 6,083,166, the entire disclosure of which is hereby incorporated by reference into this specification, discloses an ultrasound transmitter for use with a surgical device. This ultrasound transmitter may be used, e.g., to affect the anti-mitotic compound of this invention and/or tubulin and/or microtubules.

U.S. Pat. No. 6,152,882, the entire disclosure of which is hereby incorporated by reference into this specification, discloses an implantable electroporation unit, an implantable proble electrode, an implantable reference electrode, and an an amplifier unit; this electroporation unit may be used to treat, e.g., cancer cells in conjunction with the anti-mitotic compound of this invention. Claim 35 of this patent describes: “Apparatus for measurement of monophasic action potentials from an excitable tissue including a plurality of cells, the apparatus comprising: at least one probe electrode placeable adjacent to or in contact with a portion of said excitable tissue; at least one reference electrode placeable proximate said at least one probe electrode; an electroporating unit electrically connected to said at least one probe electrode and said at least one reference electrode for controllably applying to at least some of said cells subjacent said at least one probe electrode electrical current pulses suitable for causing electroporation of cell membranes of said at least some of said cells; and an amplifier unit electrically connected to said at least one probe electrode and to said at least one reference electrode for providing an output signal representing the potential difference between said probe electrode and said reference electrode.”

U.S. Pat. No. 6,169,925, the entire disclosure of which is hereby incorporated by reference into this specification, describes a transceiver for use in communication with an implantable medical device. Claim 1 of this patent describes: “An external device for use in communication with an implantable medical device, comprising: a device controller; a housing; an antenna array mounted to the housing; an RF transceiver operating at defined frequency, coupled to the antenna array; means for encoding signals to be transmitted to the implantable device, coupled to an input of the transceiver; means for decoding signals received from the implantable device, coupled to an output of the transceiver; and means for displaying the decoded signals received from the implantable device; wherein the antenna array comprises two antennas spaced a fraction of the wavelength of the defined frequency from one another, each antenna comprising two antenna elements mounted to the housing and located orthogonal to one another; and wherein the device controller includes means for selecting which of the two antennas is coupled to the transceiver.” Such a transceiver, in combination with an implantable sensor, may be used in conjunction with the anti-mitotic compound of this invention and/or tubulin and/or microtubules and/or one or more other implanted devices.

U.S. Pat. No. 6,185,452, the entire disclosure of which is hereby incorporated by reference into this specification, claims a device for stimulating internal tissue, wherein such device is comprised of: “a sealed elongate housing configured for implantation in said patient's body, said housing having an axial dimension of less than 60 mm and a lateral dimension of less than 6 mm; power consuming circuitry carried by said housing including at least one electrode extending externally of said housing, said power consuming circuitry including a capacitor and pulse control circuitry for controlling (1) the charging of said capacitor and (2) the discharging of said capacitor to produce a current pulse through said electrode; a battery disposed in said housing electrically connected to said power consuming circuitry for powering said pulse control circuitry and charging said capacitor, said battery having a capacity of at least one microwatt-hour; an internal coil and a charging circuit disposed in said housing for supplying a charging current to said battery; an external coil adapted to be mounted outside of said patient's body; and means for energizing said external coil to generate an alternating magnetic field for supplying energy to said charging circuit via said internal coil.” Such capacitative discharge energy may be used to affect either the anti-mitotic compound of this invention and/or tubulin and/or microtubules.

U.S. Pat. No. 6,235,024, the entire disclosure of which is hereby incorporated by reference into this specification, discloses an implantable high frequency energy generator; such high-frequency energy may be used to affect either the anti-mitotic compound of this invention, tubulin, microtubules, and/or one or more other implanted devices. Claim 1 of this patent describes: “A catheter system comprising: an elongate catheter tubing having a distal section, a distal end, a proximal end, and at least one lumen extending between the distal end and the proximal end; a handle attached to the proximal end of said elongate catheter tubing, wherein the handle has a cavity; an ablation element mounted at the distal section of the elongate catheter tubing, the ablation element having a wall with an outer surface and an inner surface, wherein the outer surface is covered with an outer member made of a first electrically conductive material and the inner surface is covered with an inner member made of a second electrically conductive material, and wherein the wall comprises an ultrasound transducer; an electrical conducting means having a first and a second electrical wires, wherein the first electrical wire is coupled to the outer member and the second electrical wire is coupled to the inner member of the ablation element; and a high frequency energy generator means for providing a radiofrequency energy to the ablation element through a first electrical wire of the electrical conducting means.”

An implantable light-generating apparatus is described in claim 16 of U.S. Pat. No. 6,363,279, the entire disclosure of which is hereby incorporated by reference into this specification. In one embodiment, the compound of this invention is comprised of a photolytic linker which is caused to disassociate upon being exposed to specified light energy. As is disclosed in such claim 16, this patent provides a “Heart control apparatus, comprising circuitry for generating a non-excitatory stimulus, and stimulus application devices for applying to a heart or to a portion thereof said non-excitatory stimulus, wherein the circuitry for generating a non-excitatory stimulus generates a stimulus which is unable to generate a propagating action potential and wherein said circuitry comprises a light-generating apparatus for generating light.”

An implantable ultrasound probe is described in claim 1 of U.S. Pat. No. 6,421,565, the entire disclosure of which is hereby incorporated by reference into this specification. Such ultrasound may be used, e.g., to treat the microtubules of cancer cells; and this treatment may be combined, e.g., with the anti-mitotic compounds of this invention.

Claim 1 of U.S. Pat. No. 6,421,565 describes: “An implantable cardiac monitoring device comprising: an A-mode ultrasound probe adapted for implantation in a right ventricle of a heart, said ultrasound probe emitting an ultrasound signal and receiving at least one echo of said ultrasound signal from at least one cardiac segment of the left ventricle; a unit connected to said ultrasound probe for identifying a time difference between emission of said ultrasound signal and reception of said echo and, from said time difference, determining a position of said cardiac segment, said cardiac segment having a position which, at least when reflecting said ultrasound signal, is correlated to cardiac performance, and said unit deriving an indication of said cardiac performance from said position of said cardiac segment.”

An implantable stent that contains a tube and several optical emitters located on the inner surface of the tube is disclosed in U.S. Pat. No. 6,488,704, the entire disclosure of which is hereby incorporated by reference into this specification. One may use one or more of the implantable devices described in U.S. Pat. No. 6,488,704 together with the anti-mitotic compound of this invention and/or tubulin and/or microtubules and/or another in vivo device.

Claim 1 of U.S. Pat. No. 6,488,704 describes “1. An implantable stent which comprises: (a) a tube comprising an inner surface and an outer surface, and (b) a multiplicity of optical radiation emitting means adapted to emit radiation with a wavelength from about 30 nanometers to about 30 millimeters, and a multiplicity of optical radiation detecting means adapted to detect radiation with a wavelength of from about 30 nanometers to about 30 millimeters, wherein said optical radiation emitting means and said optical radiation detecting means are disposed on the inside surface of said tube.”

Many other implantable devices and configurations are described in the claims of U.S. Pat. No. 6,488,704. These devices and configurations may be used in conjunction with the anti-mitotic compound of this invention, and/or tubulin, and/or microtubules, and/or other auxiliary, implanted deivce.

Thus, e.g., claim 2 of U.S. Pat. No. 6,488,704 discloses that the “ . . . implantable stent is comprised of a flexible casing with an inner surface and an outer surface.” claim 3 of such patent discloses that the case may be “ . . . comprised of fluoropolymer.” claim 4 of such patent discloses that the casing may be “ . . . optically impermeable.”

Thus, e.g., claim 10 of U.S. Pat. No. 6,488,704 discloses an embodiment in which an implantable stent contains “ . . . telemetry means for transmitting a signal to a receiver located external to said implantable stent.” The telemetry means may be adapted to receive “ . . . a signal from a transmitter located external to said implantable stent (see claim 11); and such signal may be a radio-frequency signal (see claims 12 and 13). The implantable stent may also comprise “ . . . telemetry means for transmitting a signal to a receiver located external to said implantable stent” (see claim 22), and/or “ . . . telemetry means for receiving a signal from a transmitter located external to said implantable stent” (see claim 23), and/or “ . . . a controller operatively connected to said means for transmitting a signal to said receiver, and operatively connected to said means for receiving a signal from said transmitter” (see claim 24).

Thus, e.g., claim 14 of U.S. Pat. No. 6,488,704 describes an implantable stent that contains a waveguide array. The waveguide array may contain “ . . . a flexible optical waveguide device” (see claim 15), and/or “ . . . means for transmitting optical energy in a specified configuration” (see claim 16), and/or “ . . . a waveguide interface for receiving said optical energy transmitted in said specified configuration by said waveguide array” (see claim 17), and/or “ . . . means for filtering specified optical frequencies” (see claim 18). The implantable stent may be comprised of “ . . . means for receiving optical energy from said waveguide array” (see claim 19), and/or “ . . . means for processing said optical energy received from waveguide array” (see claim 20). The implantable stent may comprise “ . . . means for processing said radiation emitted by said optical radiation emitting means adapted with a wavelength from about 30 nanometers to about 30 millimeters” (see claim 21).

The implantable stent of U.S. Pat. No. 6,488,404 may be comprised of implantable laser devices. Thus, e.g., and referring again to U.S. Pat. No. 6,488,704, the implantable stent may be comprised of “ . . . a multiplicity of vertical cavity surface emitting lasers and photodetectors arranged in a monolithic configuration” (see claim 27), wherein “ . . . said monolithic configuration further comprises a multiplicity of optical drivers operatively connected to said vertical cavity surface emitting lasers” (see claim 28) and/or wherein “ . . . said vertical cavity surface emitting lasers each comprise a multiplicity of distributed Bragg reflector layers” (see claim 29), and/or wherein “ . . . each of said photodetectors comprises a multiplicity of distributed Bragg reflector layers” (see claim 30), and/or wherein “ . . . each of said vertical cavity surface emitting lasers is comprised of an emission layer disposed between a first distributed Bragg reflector layer and a second distributed Bragg reflector layer” (see claim 31), and/or wherein “ . . . said emission layer is comprised of a multiplicity of quantum well structures” (see claim 32), and/or wherein “ . . . each of said photodetectors is comprised of an absorption layer disposed between a first distributed Bragg reflector layer and a second distributed Bragg reflector layer” (see claim 33), and/or wherein “ . . . each of said vertical cavity surface emitting lasers and photodetectors is disposed on a separate semiconductor substrate” (see claim 34), and/or wherein “ . . . said semiconductor substrate comprises gallium arsenide.” These devices may advantageously be used in the process of this invention.

Referring again to U.S. Pat. No. 6,488,704, the entire disclosure of which is hereby incorporated by reference into this specification, the implantable stent may be comprised of an arithmetic unit (see claim 37 of such patent), and such arithmetic unit may be “ . . . comprised of means for receiving signals from said optical radiation detecting means” (see claim 38), and/or “ . . . means for calculating the concentration of components in an analyte disposed within said implantable stent (see claim 39). In one embodiment, “said means for calculating the concentration of components in said analyte calculates concentrations of said components in said analyte based upon optimum optical path lengths for different wavelengths and values of transmitted light (see claim 40).

Referring again to U.S. Pat. No. 6,488,704, the implantable stent may contain a power supply (see claim 41 thereof) which may contain a battery (see claim 42) which, in one embodiment, is a lithium-iodine battery (see claim 43).

U.S. Pat. No. 6,585,763, the entire disclosure of which is hereby incorporated by reference into this specification, describes in its claim 1 “ . . . a vascular graft comprising: a biocompatible material formed into a shape having a longitudinal axis to enclose a lumen disposed along said longitudinal axis of said shape, said lumen positioned to convey fluid through said vascular graft; a first transducer coupled to a wall of said vascular graft; and an implantable circuit for receiving electromagnetic signals, said implantable circuit coupled to said first transducer, said first transducer configured to receive a first energy from said circuit to emit a second energy having one or more frequencies and power levels to alter said biological activity of said medication in said localized area of said body subsequent to implantation of said first transducer in said body near said localized area.” One may use the means for “ . . . altering said biological activity of said medication . . . ” in the process of this invention. The transducer may be selected from the group consisting of “ . . . an ultrasonic transducer, a plurality of light sources, an electric field transducer, an electromagnetic transducer, and a resistive heating transducer” (see claim 2), it may comprise a coil (see claim 3), it may comprise “ . . . a regular solid including piezoelectric material, and wherein a first resonance frequency, being of said one or more frequencies, is determined by a first dimension of said regular solid and a second resonance frequency, being of said one or more frequencies, is determined by a second dimension of said regular solid and further including a first electrode coupled to said regular solid and a second electrode coupled to said regular solid” (see claim 4).

U.S. Pat. No. 6,605,089, the entire disclosure of which is hereby incorporated by reference into this specification, discloses an implantable bone growth promoting device. Claim 1 of this patent describes “A device for placement into and between at least two adjacent bone masses to promote bone growth therebetween, said device comprising: an implant having opposed first and second surfaces for placement between and in contact with the adjacent bone masses, a mid-longitudinal axis, and a hollow chamber between said first and second surfaces, said hollow chamber being adapted to hold bone growth promoting material, said hollow chamber being along at least a portion of the mid-longitudinal axis of said implant, each of said first and second surfaces having at least one opening in communication with said hollow chamber into which bone from the adjacent bone masses grows; and an energizer for energizing said implant, said energizer being sized and configured to promote bone growth from adjacent bone mass to adjacent bone mass through said first and second surfaces and through at least a portion of said hollow chamber at the mid-longitudinal axis.” The implant may have a coil wrapped around it (see claim 6), a portion of the coil may be “ . . . in the form of an external thread on at least a portion of said first and second surfaces of said implant” (see claim 7), the “external thread” may be energized by the “energizer” (claim 8) by conducting “ . . . electromagnetic energy to said interior space . . . ” of the energizer (claim 9). One may use such “energizer” in the process of this invention.

Referring again to U.S. Pat. No. 6,605,089, and to the implant claimed therein, the implant may contain “ . . . a power supply delivering an electric charge” (see claim 14), and it may comprise “ . . . a first portion that is electrically conductive for delivering said electrical charge to at least a portion of the adjacent bone masses and said energizer delivers negative electrical charge to said first portion of said implant” (see claim 15). Additionally, the implant may also contain “ . . . a controller for controlling the delivery of said electric charge” that is disposed within the implant (see claim 18), that “ . . . includes one of a wave form generator and a voltage generator” (see claim 19), and that “ . . . . provides for the delivery of one of an alternating current, a direct current, and a sinusoidal current” (see claim 21).

U.S. Pat. No. 6,641,520, the entire disclosure of which is hereby incorporated by reference into this specification, discloses a magnetic field generator for providing a static or direct current magnetic field generator; the magnetic field generator described in this patent may be used in conjunction the anti-mitotic compound and/or tubulin and/or microtubules. In column 1 of this patent, some “prior art” magnetic field generators were described; and they also may be so used. It was stated in such column 1 that: “There has recently been an increased interest in therapeutic application of magnetic fields. There have also been earlier efforts of others in this area. The recent efforts, as well as those earlier made, can be categorized into three general types, based on the mechanism for generating and applying the magnetic field. The first type were what could be generally referred to as systemic applications. These were large, tubular mechanisms which could accommodate a human body within them. A patient or recipient could thus be subjected to magnetic therapy through their entire body. These systems were large, cumbersome and relatively immobile. Examples of this type of therapeutic systems included U.S. Pat. Nos. 1,418,903; 4,095,588; 5,084,003; 5,160,591; and 5,437,600. A second type of system was that of magnetic therapeutic applicator systems in the form of flexible panels, belts or collars, containing either electromagnets or permanent magnets. These applicator systems could be placed on or about portion of the recipient's body to allow application of the magnetic therapy. Because of their close proximity to the recipients body, considerations limited the amount and time duration of application of magnetic therapy. Examples of this type system were U.S. Pat. Nos. 4,757,804; 5,084,003 and 5,344,384. The third type of system was that of a cylindrical or toroidal magnetic field generator, often small and portable, into which a treatment recipient could place a limb to receive electromagnetic therapy. Because of size and other limitations, the magnetic field strength generated in this type system was usually relatively low. Also, the magnetic field was a time varying one. Electrical current applied to cause the magnetic field was time varying, whether in the form of simple alternating current waveforms or a waveform composed of a series of time-spaced pulses.”

The magnetic field generator claimed in U.S. Pat. No. 6,641,520 comprised “ . . . . a magnetic field generating coil composed of a wound wire coil generating the static magnetic field in response to electrical power; a mounting member having the coil mounted thereon and having an opening therethrough of a size to permit insertion of a limb of the recipient in order to receive electromagnetic therapy from the magnetic field coil; an electrical power supply furnishing power to the magnetic field coil to cause the coil to generate a static electromagnetic field within the opening of the mounting member for application to the recipient's limb; a level control mechanism providing a reference signal representing a specified electromagnetic field strength set point for regulating the power furnished to the magnetic field coil; a field strength sensor detecting the static electromagnetic field strength generated by the magnetic field coil and forming a field strength signal representing the detected electromagnetic field strength in the opening in the mounting member; a control signal generator receiving the field strength signal from the field strength sensor and the reference signal from the level control mechanism representing a specified electromagnetic field strength set point; and the control signal generator forming a signal to regulate the power flowing from the electrical power supply to the magnetic field coil.”

An implantable sensor is disclosed in U.S. Pat. No. 6,491,639, the entire disclosure of which is hereby incorporated by reference into this specification; this sensor also may be used in conjunction with the anti-mitotic compound of this invention, and/or tubulin, and/or microtubules. Claim 1 of such patent describes: “An implantable medical device including a sensor for use in detecting the hemodynamic status of a patient comprising: a hermetic device housing enclosing device electronics for receiving and processing data; and said device housing including at least one recess and a sensor positioned in said at least one recess.” Claim 10 of such patent describes “10. An implantable medical device including a hemodynamic sensor for monitoring arterial pulse amplitude comprising: a device housing; a transducer comprising a light source and a light detector positioned exterior to said device housing responsive to variations in arterial pulse amplitude; and wherein said light detector receives light originating from said light source and reflected from arterial vasculature of a patient and generates a signal which is indicative of variations in the reflected light caused by the expansion and contraction of said arterial vasculature. “claim 14 of such patent describes: “14. An implantable medical device including a hemodynamic sensor for monitoring arterial pulse amplitude comprising: a device housing; and an ultrasound transducer associated with said device housing responsive to variations in arterial pulse amplitude.” claim 15 of such patent describes: “15. An implantable medical device including a hemodynamic sensor for monitoring arterial pulse amplitude comprising: a device housing; and a transducer associated with said device housing responsive to variations in arterial pulse amplitude, said device housing having at least one substantially planar face and said transducer is positioned on said planar face.” claim 17 of such patent describes “ . . . an implantable pulse generator . . . ’

U.S. Pat. No. 6,663,555, the entire disclosure of which is incorporated by reference into this specification, also claims a magnetic field generator; this magnetic field generator may be used in conjunction with the anti-mitotic compound of this invention and/or tubulin and/or microtubules. Claim 1 of this patent describes: “A magnet keeper-shield assembly for housing a magnet, said magnet keeper-shield assembly comprising: a keeper-shield comprising a material substantially permeable to a magnetic flux; a cavity in the keeper-shield, said cavity comprising an inner side wall and a base, and said cavity being adapted to accept a magnet having a front and a bottom face; an actuator extending through the base; a plurality of springs extending through the base, said springs operative to exert a force in a range from about 175 pounds to about 225 pounds on the bottom face of the magnet in a retracted position, and wherein said magnet produces at least about 118 gauss at a distance of about 10 cm from the front face in the extended position and produces at most about 5 gauss at a distance less than or equal to about 22 cm from the front face in the retracted position.”

Published United States patent application U.S. 2002/0182738 discloses an implantable flow cytometer; the entire disclosure of this published United States patent application is hereby incorporated by reference into this specification. Claim 1 of this patent describes “A flow cytometer comprising means for sampling cellular material within a body, means for marking cells within said bodily fluid with a marker to produce marked cells, means for analyzing said marked cells, a first means for removing said marker from said marked cells, a second means for removing said marker from said marked cells, means for sorting said cells within said bodily fluid to produce sorted cells, and means for maintaining said sorted cells cells in a viable state.”

Referring again to published United States patent application U.S. 2002/0182738, the implantable flow cytometer may contain “ . . . a first control valve operatively connected to said first means for removing said marker from said marked cells and to said second means for removing said marker from said marked cells . . . ” (see claim 3), a controller connected to the first control valve (claim 4), a second control valve (claim 5), a third control valve (claim 6), a dye separator (claims 7 and 8), an analyzer for testing blood purity (claim 9), etc.

A similar flow cytometer is disclosed in published United States patent application U.S. 2003/0036718, the entire disclosure of which is also hereby incorporated by reference into this specification.

Published United States patent application U.S. 2003/0036776, the entire disclosure of which is hereby incorporated by reference into this specification, discloses an MRI-compatible implantable device. Claim 1 of this patent describes “A cardiac assist device comprising means for connecting said cardiac assist device to a heart, means for furnishing electrical impulses from said cardiac assist device to said heart, means for ceasing the furnishing of said electrical impulses to said heart, means for receiving pulsed radio frequency fields, means for transmitting and receiving optical signals, and means for protecting said heart and said cardiac assist device from currents induced by said pulsed radio frequency fields, wherein said cardiac assist device contains a control circuit comprised of a parallel resonant frequency circuit and means for activating said parallel resonant frequency circuit.” The “ . . . means for activating said parallel resonant circuit . . . ” may contain “ . . . comprise optical means (see claim 2) such as an optical switch (claim 3) comprised of “ . . . a pin type diode . . . ” (claim 4) and connected to an optical fiber (claim 5). The optical switch may be “ . . . activated by light from a light source . . . ” (claim 6), and it may be located with a biological organism (claim 7). The light source may be located within the biological organism (claim 9), and it may provide “ . . . light with a wavelength of from about 750 to about 850 nanometers . . . .”

Polymeric Carriers and/or Delivery Systems

The anti-mitotic compound of this invention may be used in conjunction with prior art polymeric carriers and/or delivery systems comprised of polymeric material. In one embodiment, the polymeric material is preferably comprised of one or more anti-mitotic compounds that are adapted to be released from the polymeric material when the polymeric material is disposed within a biological organism. The polymeric material may be, e.g., any of the drug eluting polymers known to those skilled in the art.

By way of illustration, and referring to U.S. Pat. No. 3,279,996 (the entire disclosure of which is hereby incorporated by reference into this specification), the polymeric material may be silicone rubber. This patent claims “An implantate for releasing a drug in the tissues of a living organism comprising a drug enclosed in a capsule of silicone rubber, . . . said drug being soluble in and capable of diffusing through said silicone rubber to the outer surface of said capsule . . . .” One may use, as the anti-mitotic compound a material that is soluble in and capable of diffusing through the polymeric material.

At column 1 of U.S. Pat. No. 3,279,996, other “carrier agents” which may be used as polymeric material are also disclosed, including “ . . . beeswax, peanut oil, stearates, etc.” Any of these “carrier agents” may be used as the polymeric material.

By way of further illustration, and as is disclosed in U.S. Pat. No. 4,191,741 (the entire disclosure of which is hereby incorporated by reference into this specification), one may use dimethylpolsiloxane rubber as the polymeric material. This patent claims “A solid, cylindrical, subcutaneous implant for improving the rate of weight gain of ruminant animals which comprises (a) a biocompatible inert core having a diameter of from about 2 to about 10 mm. and (b) a biocompatible coating having a thickness of from about 0.2 to about 1 mm., the composition of said coating comprising from about 5 to about 40 percent by weight of estradiol and from about 95 to about 60 percent by weight of a dimethylpolysiloxane rubber.”

In column 1 of U.S. Pat. No. 4,191,741, other materials which may be used as the polymeric material are disclosed. Thus, it is stated in such patent that “Long et al. U.S. Pat. No. 3,279,996 describes an implant for releasing a drug in the tissues of a living organism comprising the drug enclosed in a capsule formed of silicone rubber. The drug migrates through the silicone rubber wall and is slowly released into the living tissues. A number of biocompatible silicone rubbers are described in the Long et al. patent. When a drug delivery system such as that described in U.S. Pat. No. 3,279,996 is used in an effort to administer estradiol to a ruminant animal a number of problems are encountered. For example, an excess of the drug is generally required in the hollow cavity of the implant. Also, it is difficult to achieve a constant rate of administration of the drug over a long time period such as from 200 to 400 days as would be necessary for the daily administration of estradiol to a growing beef animal. Katz et al. U.S. Pat. No. 4,096,239 describes an implant pellet containing estradiol or estradiol benzoate which has an inert spherical core and a uniform coating comprising a carrier and the drug. The coating containing the drug must be both biocompatible and biosoluble, i.e., the coating must dissolve in the body fluids which act upon the pellet when it is implanted in the body. The rate at which the coating dissolves determines the rate at which the drug is released. Representative carriers for use in the coating material include cholesterol, solid polyethylene glycols, high molecular weight fatty acids and alcohols, biosoluble waxes, cellulose derivatives and solid polyvinyl pyrrolidone.” The polymeric material used with the anti-mitotic compound is, in one embodiment, both biocompatible and biosoluble.

By way of yet further illustration, and referring to U.S. Pat. No. 4,429,080 (the entire disclosure of which is hereby incorporated by reference into this specification), the polymeric material may be a synthetic absorbable copolymer formed by copolymerizing glycolide with trimethylene carbonate.

By way of yet further illustration, and referring to U.S. Pat. No. 4,581,028 (the entire disclosure of which is hereby incorporated by reference into this specification), the polymeric material may be selected from the group consisting of polyester (such as Dacron), polytetrafluoroethylene, polyurethane silicone-based material, and polyamide. The polymeric material of this patent is comprised “ . . . of at least one antimicrobial agent selected from the group consisting of the metal salts of sulfonamides.” In one embodiment, the polymeric material is comprised of an antimicrobial agent.

By way of yet further illustration, and referring to U.S. Pat. No. 4,481,353, (the entire disclosure of which is hereby incorporated by reference into this specification), the polymeric material may be the bioresorbable polyester disclosed in such patent. U.S. Pat. No. 4,481,353 claims “A bioresorbable polyester in which monomeric subunits are arranged randomly in the polyester molecules, said polyester comprising the condensation reaction product of a Krebs Cycle dicarboxylic acid or isomer or anhydride thereof, chosen for the group consisting of succinic acid, fumaric acid, oxaloacetic acid, L-malic acid, and D-malic acid, a diol having 2, 4, 6, or 8 carbon atoms, and an alpha-hydroxy carboxylic acid chosen from the group consisting of glycolic acid, L-lactic acid and D-lactic acid.”

By way of yet further illustration, and referring to U.S. Pat. No. 4,846,844 (the entire disclosure of which is hereby incorporated by reference into this specification), the polymeric material may be a silicone polymer matrix in which an anabolic agent (such as an anabolic steroid, or estradiol) is disposed. This patent claims “An implant adapted for the controlled release of an anabolic agent, said implant comprising a silicone polymer matrix, an anabolic agent in said polymer matrix, and an antimicrobial coating, wherein the coating comprises a first-applied non-vulcanizing silicone fluid and a subsequently applied antimicrobial agent in contact with said fluid.”

By way of yet further illustration, and referring to U.S. Pat. No. 4,916,193 (the entire disclosure of which is hereby incorporated by reference into this specification), the polymeric material may be a copolymer containing carbonate repeat units and ester repeat units (see, e.g., claim 1 of the patent). As disclosed in column 2 of the patent, it may also be “collagen,” “homopolymers and copolymers of glycolic acid and lactic acid,” “alpha-hydroxy carboxylic acids in conjunction with Krebs cycle dicarboxylic acids and aliphatic diols,” “polycarbonate-containing polymers,” and “high molecular weight fiber-forming crystalline copolymers of lactide and glycolide.” Thus, it is disclosed in such column 2 that: “Various polymers have been proposed for use in the fabrication of bioresorbable medical devices. Examples of absorbable materials used in nerve repair include collagen as disclosed by D. G. Kline and G. J. Hayes, “The Use of a Resorbable Wrapper for Peripheral Nerve Repair, Experimental Studies in Chimpanzees”, J. Neurosurgery 21, 737 (1964). Artandi et al., U.S. Pat. No. 3,272,204 (1966) reports the use of collagen protheses that are reinforced with nonabsorbable fabrics. These articles are intended to be placed permanently in a human body. However, one of the disadvantages inherent with collagenous materials, whether utilized alone or in conjunction with biodurable materials, is their potential antigenicity. Other biodegradable polymers of particular interest for medical implantation purposes are homopolymers and copolymers of glycolic acid and lactic acid. A nerve cuff in the form of a smooth, rigid tube has been fabricated from a copolymer of lactic and glycolic acids [The Hand; 10 (3) 259 (1978)]. European patent application No. 118-458-A discloses biodegradable materials used in organ protheses or artificial skin based on poly-L-lactic acid and/or poly-DL-lactic acid and polyester or polyether urethanes. U.S. Pat. No. 4,481,353 discloses bioresorbable polyester polymers, and composites containing these polymers, that are also made up of alpha-hydroxy carboxylic acids, in conjunction with Krebs cycle dicarboxylic acids and aliphatic diols. These polyesters are useful in fabricating nerve guidance channels as well as other surgical articles such as sutures and ligatures. U.S. Pat. Nos. 4,243,775 and 4,429,080 disclose the use of polycarbonate-containing polymers in certain medical applications, especially sutures, ligatures and haemostatic devices. However, this disclosure is clearly limited only to “AB” and “ABA” type block copolymers where only the “B” block contains poly(trimethylene carbonate) or a random copolymer of glycolide with trimethylene carbonate and the “A” block is necessarily limited to glycolide. In the copolymers of this patent, the dominant portion of the polymer is the glycolide component. U.S. Pat. No. 4,157,437 discloses high molecular weight, fiber-forming crystalline copolymers of lactide and glycolide which are disclosed as useful in the preparation of absorbable surgical sutures. The copolymers of this patent contain from about 50 to 75 wt. % of recurring units derived from glycolide.”

By way of further illustration, and referring to U.S. Pat. No. 5,176,907 (the entire disclosure of which is hereby incorporated by reference into this specification), the polymeric material may be the poly-phosphoester-urethane) described and claimed in claim 1 of such patent. Furthermore, the polymeric material may be one or more of the biodegradable polymers discussed in columns 1 and 2 of such patent. As is disclosed in such columns 1 and 2: “Polymers have been used as carriers of therapeutic agents to effect a localized and sustained release (Controlled Drug Delivery, Vol. I and II, Bruck, S. D., (ed.), CRC Press, Boca Raton, Fla., 1983; Leong, et al., Adv. Drug Delivery Review, 1:199, 1987). These anti-mitotic compound delivery systems simulate infusion and offer the potential of enhanced therapeutic efficacy and reduced systemic toxicity.” The polymeric material may be such a poly-phosphoester-urethane.

U.S. Pat. No. 5,176,907 also discloses “For a non-biodegradable matrix, the steps leading to release of the anti-mitotic compound are water diffusion into the matrix, dissolution of the therapeutic agent, and out-diffusion of the anti-mitotic compound through the channels of the matrix. As a consequence, the mean residence time of the anti-mitotic compound existing in the soluble state is longer for a non-biodegradable matrix than for a biodegradable matrix where a long passage through the channels is no longer required. Since many pharmaceuticals have short half-lives it is likely that the anti-mitotic compound is decomposed or inactivated inside the non-biodegradable matrix before it can be released. This issue is particularly significant for many bio-macromolecules and smaller polypeptides, since these molecules are generally unstable in buffer and have low permeability through polymers. In fact, in a non-biodegradable matrix, many bio-macromolecules will aggregate and precipitate, clogging the channels necessary for diffusion out of the carrier matrix. This problem is largely alleviated by using a biodegradable matrix which allows controlled release of the therapeutic agent. Biodegradable polymers differ from non-biodegradable polymers in that they are consumed or biodegraded during therapy. This usually involves breakdown of the polymer to its monomeric subunits, which should be biocompatible with the surrounding tissue. The life of a biodegradable polymer in vivo depends on its molecular weight and degree of cross-linking; the greater the molecular weight and degree of crosslinking, the longer the life. The most highly investigated biodegradable polymers are polylactic acid (PLA), polyglycolic acid (PGA), polyglycolic acid (PGA), copolymers of PLA and PGA, polyamides, and copolymers of polyamides and polyesters. PLA, sometimes referred to as polylactide, undergoes hydrolytic de-esterification to lactic acid, a normal product of muscle metabolism. PGA is chemically related to PLA and is commonly used for absorbable surgical sutures, as is the PLA/PGA copolymer. However, the use of PGA in controlled-release implants has been limited due to its low solubility in common solvents and subsequent difficulty in fabrication of devices.” The polymeric material 14 may be a biodegradable polymeric material.

U.S. Pat. No. 5,176,907 also discloses “An advantage of a biodegradable material is the elimination of the need for surgical removal after it has fulfilled its mission. The appeal of such a material is more than simply for convenience. From a technical standpoint, a material which biodegrades gradually and is excreted over time can offer many unique advantages.”

U.S. Pat. No. 5,176,907 also discloses “A biodegradable thereapeutic agent delivery system has several additional advantages: 1) the therapeutic agent release rate is amenable to control through variation of the matrix composition; 2) implantation can be done at sites difficult or impossible for retrieval; 3) delivery of unstable therapeutic agents is more practical. This last point is of particular importance in light of the advances in molecular biology and genetic engineering which have lead to the commercial availability of many potent bio-macromolecules. The short in vivo half-lives and low GI tract absorption of these polypeptides render them totally unsuitable for conventional oral or intravenous administration. Also, because these substances are often unstable in buffer, such polypeptides cannot be effectively delivered by pumping devices.”

U.S. Pat. No. 5,176,907 also discloses “In its simplest form, a biodegradable therapeutic agent delivery system consist of a dispersion of the drug solutes in a polymer matrix. The therapeutic agent is released as the polymeric matrix decomposes, or biodegrades into soluble products which are excreted from the body. Several classes of synthetic polymers, including polyesters (Pitt, et al., in Controlled Release of Bioactive Materials, R. Baker, Ed., Academic Press, New York, 1980); polyamides (Sidman, et al., Journal of Membrane Science, 7:227, 1979); polyurethanes (Maser, et al., Journal of Polymer Science, Polymer Symposium, 66:259, 1979); polyorthoesters (Heller, et al., Polymer Engineering Science, 21:727, 1981); and polyanhydrides (Leong, et al., Biomaterials, 7:364, 1986) have been studied for this purpose.” The “therapeutic agent” used in this (and other) patents may be the anti-mitotic compound of this invention.

By way of yet further illustration, and referring to U.S. Pat. No. 5,194,581 (the entire disclosure of which is hereby incorporated by reference into this specification), the polymeric material may the poly (phosphoester) compositions described in such patent.

The polymeric material may be in the form of microcapsules within which the anti-mitotic compound of this invention is disposed. Thus, one may use microcapusels such as, e.g., the microcapsule described in U.S. Pat. No. 6,117,455, the entire disclosure of which is hereby incorporated by reference into this specification. As is disclosed in the abstract of this patent, there is provided “A sustained-release microcapsule contains an amorphous water-soluble pharmaceutical agent having a particle size of from 1 nm-10 μm and a polymer. The microcapsule is produced by dispersing, in an aqueous phase, a dispersion of from 0.001-90% (w/w) of an amorphous water-soluble pharmaceutical agent in a solution of a polymer having a wt. avg. molecular weight of 2,000-800,000 in an organic solvent to prepare an s/o/w emulsion and subjecting the emulsion to in-water drying.”

In one embodiment, disclosed in U.S. Pat. No. 5,484,584 (the entire disclosure of which is hereby incorporated by reference into this specification), a poly (benzyl-L-glutamate) microsphere is disclosed (see, e.g., claim 10); the anti-mitotic compound of this invention may be disposed within and/or on the surface of such microsphere. As is disclosed in the abstract of this patent, “The present invention relates to a highly efficient method of preparing modified microcapsules exhibiting selective targeting. These microcapsules are suitable for encapsulation surface attachment of therapeutic and diagnostic agents. In one aspect of the invention, surface charge of the polymeric material is altered by conjugation of an amino acid ester to the providing improved targeting of encapsulated agents to specific tissue cells. Examples include encapsulation of radiodiagnostic agents in 1 μm capsules to provide improved opacification and encapsulation of cytotoxic agents in 100 μm capsules for chemoembolization procedures. The microcapsules are suitable for attachment of a wide range of targeting agents, including antibodies, steroids and drugs, which may be attached to the microcapsule polymer before or after formation of suitably sized microcapsules. The invention also includes microcapsules surface modified with hydroxyl groups. Various agents such as estrone may be attached to the microcapsules and effectively targeted to selected organs.”

The release rate of the anti-mitotic compound from the polymeric material may be varied in, e.g., the manner suggested in column 6 of U.S. Pat. No. 5,194,581, the entire disclosure of which is hereby incorporated by reference into this specification. As is disclosed in such column 6, “A wide range of degradation rates can be obtained by adjusting the hydrophobicities of the backbones of the polymers and yet the biodegradability is assured. This can be achieved by varying the functional groups R or R′. The combination of a hydrophobic backbone and a hydrophilic linkage also leads to heterogeneous degradation as cleavage is encouraged, but water penetration is resisted.” As is disclosed at column 9 of such patent, “The rate of biodegradation of the poly(phosphoester) compositions of the invention may also be controlled by varying the hydrophobicity of the polymer. The mechanism of predictable degradation preferably relies on either group R′ in the poly(phosphoester) backbone being hydrophobic for example, an aromatic structure, or, alternatively, if the group R′ is not hydrophobic, for example an aliphatic group, then the group R is preferably aromatic. The rates of degradation for each poly(phosphoester) composition are generally predictable and constant at a single pH. This permits the compositions to be introduced into the individual at a variety of tissue sites. This is especially valuable in that a wide variety of compositions and devices to meet different, but specific, applications may be composed and configured to meet specific demands, dimensions, and shapes—each of which offers individual, but different, predictable periods for degradation. When the composition of the invention is used for long term delivery of a anti-mitotic compound a relatively hydrophobic backbone matrix, for example, containing bisphenol A, is preferred. It is possible to enhance the degradation rate of the poly(phosphoester) or shorten the functional life of the device, by introducing hydrophilic or polar groups, into the backbone matrix. Further, the introduction of methylene groups into the backbone matrix will usually increase the flexibility of the backbone and decrease the crystallinity of the polymer. Conversely, to obtain a more rigid backbone matrix, for example, when used orthopedically, an aromatic structure, such as a diphenyl group, can be incorporated into the matrix. Also, the poly(phosphoester) can be crosslinked, for example, using 1,3,5-trihydroxybenzene or (CH2 OH)4 C, to enhance the modulus of the polymer. Similar considerations hold for the structure of the side chain (R).”

,By way of yet further illustration, and referring to U.S. Pat. No. 5,252,713 (the entire disclosure of which is hereby incorporated by reference into this specification), the polymeric material may be a polypeptide comprising at least one drug-binding domain that non-covalently binds a drug. The means of identifying and isolating such a polypeptide is described at columns 5-7 of the patent, wherein it is disclosed that: “The process of isolating a polymeric carrier from a drug-binding, large molecular weight protein begins with the identification of a large protein that can non-covalently bind the drug of interest. Examples of such protein/drug pairs are shown in Table I. The drugs in the Table (other than the steroids) are anti-cancer drugs . . . .”

As is also disclosed in U.S. Pat. No. 5,252,713, “Other drug-binding proteins may be identified by appropriate analytical procedures, including Western blotting of large proteins or protein fragments and subsequent incubation with a detectable form of drug. Alternative procedures include combining a drug and a protein in a solution, followed by size exclusion HPLC gel filtration, thin-layer chromatography (TLC), or other analytical procedures that can discriminate between free and protein-bound drug. Detection of drug binding can be accomplished by using radiolabeled, fluorescent, or colored drugs and appropriate detection methods. Equilibrium dialysis with labeled drug may be used. Alternative methods include monitoring the fluorescence change that occurs upon binding of certain drugs (e.g., anthracyclines or analogs thereof, which should be fluorescent) . . . ”. In one detection method, drug and protein are mixed, and an aliquot of this solution (not exceeding 5% of the column volume of an HPLC column, such as a Bio-sil TSK-250 7.5×30 cm column) is loaded onto the HPLC column. The flow rate is 1 ml/min. The drug bound to protein will elute first, in a separate peak, followed by free drug, eluting at a position characteristic of its molecular weight. If the drug is doxorubicin, both a 280-nm as well as a 495-nm adsorptive peak will correspond to the elution position of the protein if interaction occurs. The elution peaks for other drugs will indicate whether drug binding occurs . . . .”

As is also disclosed in U.S. Pat. No. 5,252,713, “Knowledge of the chemical structure of a particular drug (i.e., whether chemically reactive functional groups are present) allows one to predict whether covalent binding of the drug to a given protein can occur. Additional methods for determining whether drug binding is covalent or non-covalent include incubating the drug with the protein, followed by dialysis or subjecting the protein to denaturing conditions. Release of the drug from the drug-binding protein during these procedures indicates that the drug was non-covalently bound. Usually, a dissociation constant of about 10-15 M or less indicates covalent or extremely tight non-covalent binding . . . .”

As is also disclosed in U.S. Pat. No. 5,252,713, “During dialysis, non-covalently bound drug molecules are released over time from the protein and pass through a dialysis membrane, whereas covalently bound drug molecules are retained on the protein. An equilibrium constant of about 10-5 M indicates non-covalent binding. Alternatively, the protein may be subjected to denaturing conditions; e.g., by gel electrophoresis on a denaturing (SDS) gel or on a gel filtration column in the presence of a strong denaturant such as 6M guanidine. Covalently bound drug molecules remain bound to the denatured protein, whereas non-covalently bound drug molecules are released and migrate separately from the protein on the gel and are not retained with the protein on the column.”

As is also disclosed in U.S. Pat. No. 5,252,713, “Once a protein that can non-covalently bind a particular drug of interest is identified, the drug-binding domain is identified and isolated from the protein by any suitable means. Protein domains are portions of proteins having a particular function or activity (in this case, non-covalent binding of drug molecules). The present invention provides a process for producing a polymeric carrier, comprising the steps of generating peptide fragments of a protein that is capable of non-covalently binding a drug and identifying a drug-binding peptide fragment, which is a peptide fragment containing a drug-binding domain capable of non-covalently binding the drug, for use as the polymeric carrier.”

As is also disclosed in U.S. Pat. No. 5,252,713, “One method for identifying the drug-binding domain begins with digesting or partially digesting the protein with a proteolytic enzyme or specific chemicals to produce peptide fragments. Examples of useful proteolytic enzymes include Iys-C-endoprotease, arg-C-endoprotease, V8 protease, endoprolidase, trypsin, and chymotrypsin. Examples of chemicals used for protein digestion include cyanogen bromide (cleaves at methionine residues), hydroxylamine (cleaves the Asn-Gly bond), dilute acetic acid (cleaves the Asp-Pro bond), and iodosobenzoic acid (cleaves at the tryptophane residue). In some cases, better results may be achieved by denaturing the protein (to unfold it), either before or after fragmentation.”

As is also disclosed in U.S. Pat. No. 5,252,713, “The fragments may be separated by such procedures as high pressure liquid chromatography (HPLC) or gel electrophoresis. The smallest peptide fragment capable of drug binding is identified using a suitable drug-binding analysis procedure, such as one of those described above. One such procedure involves SDS-PAGE gel electrophoresis to separate protein fragments, followed by Western blotting on nitrocellulose, and incubation with a colored drug like adriamycin. The fragments that have bound the drug will appear red. Scans at 495 nm with a laser densitometer may then be used to analyze (quantify) the level of drug binding.”

As is also disclosed in U.S. Pat. No. 5,252,713, “Preferably, the smallest peptide fragment capable of non-covalent drug binding is used. It may occasionally be advisable, however, to use a larger fragment, such as when the smallest fragment has only a low-affinity drug-binding domain.”

As is also disclosed in U.S. Pat. No. 5,252,713, “The amino acid sequence of the peptide fragment containing the drug-binding domain is elucidated. The purified fragment containing the drug-binding region is denatured in 6M guanidine hydrochloride, reduced and carboxymethylated by the method of Crestfield et al., J. Biol. Chem. 238:622, 1963. As little as 20 to 50 picomoles of each peptide fragment can be analyzed by automated Edman degradation using a gas-phase or liquidpulsed protein sequencer (commercially available from Applied Biosystems, Inc.). If the peptide fragment is longer than 30 amino acids, it will most likely have to be fragmented as above and the amino acid sequence patched together from sequences of overlapping fragments.”

As is also disclosed in U.S. Pat. No. 5,252,713, “Once the amino acid sequence of the desired peptide fragment has been determined, the polymeric carriers can be made by either one of two types of synthesis. The first type of synthesis comprises the preparation of each peptide chain with a peptide synthesizer (e.g., commercially available from Applied Biosystems). The second method utilizes recombinant DNA procedures.” The polymeric material 14 may comprise one or more of the polymeric carriers described in U.S. Pat. No. 5,252,713.

As is also disclosed in U.S. Pat. No. 5,252,713, “Peptide amides can be made using 4-methylbenzhydrylamine-derivatized, cross-linked polystyrene-1% divinylbenzene resin and peptide acids made using PAM (phenylacetamidomethyl) resin (Stewart et al., “Solid Phase Peptide Synthesis,” Pierce Chemical Company, Rockford, Ill., 1984). The synthesis can be accomplished either using a commercially available synthesizer, such as the Applied Biosystems 430A, or manually using the procedure of Merrifield et al., Biochemistry 21:5020-31, 1982; or Houghten, PNAS 82:5131-35, 1985. The side chain protecting groups are removed using the Tam-Merrifield low-high HF procedure (Tam et al., J. Am. Chem. Soc. 105:6442-55, 1983). The peptide can be extracted with 20% acetic acid, lyophilized, and purified by reversed-phase HPLC on a Vydac C-4 Analytical Column using a linear gradient of 100% water to 100% acetonitrile-0.1% trifluoroacetic acid in 50 minutes. The peptide is analyzed using PTC-amino acid analysis (Heinrikson et al., Anal. Biochem. 136:65-74, 1984). After gas-phase hydrolysis (Meltzer et al., Anal. Biochem. 160: 356-61, 1987), sequences are confirmed using the Edman degradation or fast atom bombardment mass spectroscopy. After synthesis, the polymeric carriers can be tested for drug binding using size-exclusion HPLC, as described above, or any of the other analytical methods listed above.”

The polymeric carriers of U.S. Pat. No. 5,252,713 may be used with the anti-mitotic compounds of this invention. As is also disclosed in U.S. Pat. No. 5,252,713, “The polymeric carriers of the present invention preferably comprise more than one drug-binding domain. A polypeptide comprising several drug-binding domains may be synthesized. Alternatively, several of the synthesized drug-binding peptides may be joined together using bifunctional cross-linkers, as described below.” The polymeric material in one embodiment, comprises more than one drug-binding domain.

By way of yet further illustration, and referring to U.S. Pat. No. 5,420,105 (the entire disclosure of which is hereby incorporated by reference into this specification), the polymeric material may form a conjugate with a ligand. Thus, and referring to claim 1 of such patent, such conjugate may be “A ligand or an anti-ligand/polymeric carrier/drug conjugate comprising a ligand consisting of biotin or an anti-ligand selected from the group consisting of avidin and streptavidin, which ligand or anti-ligand is covalently bound to a polymeric carrier that comprises at least one drug-binding domain derived from a drug-binding protein, and at least one drug non-covalently bound to the polymeric carrier, wherein the polymeric carrier does not comprise an entire drug-binding protein, but is derived from a drug-binding domain of said drug-binding protein which derivative non-covalently binds a drug which is non-covalently bound by an entire naturally occurring drug-binding protein, and wherein the molecular weight of the polymeric carrier is less than about 60,000 daltons, and wherein said drug is selected from the group consisting of an anti-cancer anthracycline antibiotic, cis-platinum, methotrexate, vinblastine, mitoxanthrone ARA-C, 6-mercaptopurine, 6-mercaptoguanosine, mytomycin C and a steroid.”

The polymeric material form comprise a reservoir (see U.S. Pat. No. 5,447,724) for the anti-mitotic compound(s). Such a reservoir may be constructed in accordance with the procedure described in U.S. Pat. No. 5,447,724, which claims “A medical device at least a portion of which comprises: a body insertable into a patient, said body having an exposed surface which is adapted for exposure to tissue of a patient and constructed to release, at a predetermined rate, therapeutic agent to inhibit adverse physiological reaction of said tissue to the presence of the body of said medical device, said therapeutic agent selected from the group consisting of antithrombogenic agents, antiplatelet agents, prostaglandins, thrombolytic drugs, antiproliferative drugs, antirejection drugs, antimicrobial drugs, growth factors, and anticalcifying agents, at said exposed surface, said body including: an outer polymer metering layer, and an internal polymer layer underlying and supporting said outer polymer metering layer and in intimate contact therewith, said internal polymer layer defining a reservoir for said therapeutic agent, said reservoir formed by a polymer selected from the group consisting of polyurethanes and its copolymers, silicone and its copolymers, ethylene vinylacetate, thermoplastic elastomers, polyvinylchloride, polyolefins, cellulosics, polyamides, polytetrafluoroethylenes, polyesters, polycarbonates, polysulfones, acrylics, and acrylonitrile butadiene styrene copolymers, said outer polymer metering layer having a stable, substantially uniform, predetermined thickness covering the underlying reservoir so that no portion of the reservoir is directly exposed to body fluids and incorporating a distribution of an elutable component which, upon exposure to body fluid, elutes from said outer polymer metering layer to form a predetermined porous network capable of exposing said anti-mitotic compoundin said reservoir in said internal polymer layer to said body fluid, said elutable component is selected from the group consisting of polyethylene oxide, polyethylene glycol, polyethylene oxide/polypropylene oxide copolymers, polyhydroxyethylmethacrylate, polyvinylpyrollidone, polyacrylamide and its copolymers, liposomes, albumin, dextran, proteins, peptides, polysaccharides, polylactides, polygalactides, polyanhydrides, polyorthoesters and their copolymers, and soluble cellulosics, said reservoir defined by said internal polymer layer incorporating said therapeutic agent in a manner that permits substantially free outward release of said therapeutic agent from said reservoir into said porous network of said outer polymer metering layer as said elutable component elutes from said polymer metering layer, said predetermined thickness and the concentration and particle size of said elutable component being selected to enable said outer polymer metering layer to meter the rate of outward migration of the thereapuetic agent from said internal reservoir layer through said outer polymer metering layer, said outer polymer metering layer and said internal polymer layer, in combination, enabling prolonged controlled release, at said predetermined rate, of said therapeutic agent at an effective dosage level from said exposed surface of said body of said medical device to the tissue of said patient to inhibit adverse reaction of the patient to the prolonged presence of said body of said medical device in said patient.”

U.S. Pat. No. 5,447,724 also discloses the preparation of the “reservoir” in e.g., in columns 8 and 9 of the patent, wherein it is disclosed that: “A particular advantage of the time-release polymers of the invention is the manufacture of coated articles, i.e., medical instruments. Referring now to FIG. 3, the article to be coated such as a catheter 50 may be mounted on a mandrel or wire 60 and aligned with the preformed apertures 62 (slightly larger than the catheter diameter) in the teflon bottom piece 63 of a boat 64 that includes a mixture 66 of polymer at ambient temperature, e.g., 25° C. To form the reservoir portion, the mixture may include, for example, nine parts solvent, e.g. tetrahydrofuran (THF), and one part Pellthane® polyurethane polymer which includes the desired proportion of ground sodium heparin particles. The boat may be moved in a downward fashion as indicated by arrow 67 to produce a coating 68 on the exterior of catheter 50. After a short (e.g., 15 minutes) drying period, additional coats may be added as desired. After coating, the catheter 50 is allowed to air dry at ambient temperature for about two hours to allow complete solvent evaporation and/or polymerization to form the reservoir portion. For formation of the surface-layer the boat 64 is cleaned of the reservoir portion mixture and filled with a mixture including a solvent, e.g. THF (9 parts) and Pellthane® (1 part) having the desired amount of elutable component. The boat is moved over the catheter and dried, as discussed above to form the surface-layer. Subsequent coats may also be formed. An advantage of the dipping method and apparatus described with regard to FIG. 3is that highly uniform coating thickness may be achieved since each portion of the substrate is successively in contact with the mixture for the same period of time and further, no deformation of the substrate occurs. Generally, for faster rates of movement of the boat 64, thicker layers are formed since the polymer gels along the catheter surfaces upon evaporation of the solvent, rather than collects in the boat as happens with slower boat motion. For thin layers, e.g., on the order of a few mils, using a fairly volatile solvent such as THF, the dipping speed is generally between 26 to 28 cn/min for the reservoir portion and around 21 cm/min for the outer layer for catheters in the range of 7 to 10 F. The thickness of the coatings may be calculated by subtracting the weight of the coated catheter from the weight of the uncoated catheter, dividing by the calculated surface area of the uncoated substrate and dividing by the known density of the coating. The solvent may be any solvent that solubilizes the polymer and preferably is a more volatile solvent that evaporates rapidly at ambient temperature or with mild heating. The solvent evaporation rate and boat speed are selected to avoid substantial solubilizing of the catheter substrate or degradation of a prior applied coating so that boundaries between layers are formed.”

By way of yet further illustration, and referring to U.S. Pat. No. 5,464,650 (the entire disclosure of which is hereby incorporated by reference into this specification), the polymeric material may be one or ore of the polymeric materials discussed at columns 4 and 5 of such patent. Referring to such columns 4 and 5, it is disclosed that: “The polymer chosen must be a polymer that is biocompatible and minimizes irritation to the vessel wall when the stent is implanted. The polymer may be either a biostable or a bioabsorbable polymer depending on the desired rate of release or the desired degree of polymer stability, but a bioabsorbable polymer is probably more desirable since, unlike a biostable polymer, it will not be present long after implantation to cause any adverse, chronic local response. Bioabsorbable polymers that could be used include poly(L-lactic acid), polycaprolactone, poly(lactide-co-glycolide), poly(hydroxybutyrate), poly(hydroxybutyrate-co-valerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters) (e.g. PEO/PLA), polyalkylene oxalates, polyphosphazenes and biomolecules such as fibrin, fibrinogen, cellulose, starch, collagen and hyaluronic acid. Also, biostable polymers with a relatively low chronic tissue response such as polyurethanes, silicones, and polyesters could be used and other polymers could also be used if they can be dissolved and cured or polymerized on the stent such as polyolefins, polyisobutylene and ethylene-alphaolefin copolymers; acrylic polymers and copolymers, vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile, polyvinyl ketones; polyvinyl aromatics, such as polystyrene, polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; alkyd resins; polycarbonates; polyoxymethylenes; polyimides; polyethers; epoxy resins, polyurethanes; rayon; rayon-triacetate; cellulose, cellulose acetate, cellulose butyrate; cellulose acetate butyrate; cellophane; cellulose nitrate; cellulose propionate; cellulose ethers; and carboxymethyl cellulose. The ratio of therapeutic substance to polymer in the solution will depend on the efficacy of the polymer in securing the therapeutic substance onto the stent and the rate at which the coating is to release the therapeutic substance to the tissue of the blood vessel. More polymer may be needed if it has relatively poor efficacy in retaining the therapeutic substance on the stent and more polymer may be needed in order to provide an elution matrix that limits the elution of a very soluble therapeutic substance. A wide ratio of therapeutic substance to polymer could therefore be appropriate and could range from about 10:1 to about 1:100.”

By way of yet further illustration, and referring to U.S. Pat. No. 5,470,307 (the entire disclosure of which is hereby incorporated by reference into this specification), the polymeric material may a synthetic or natural polymer, such as polyamide, polyester, polyolefin (polypropylene or polyethylene), polyurethane, latex, acrylamide, methacrylate, polyvinylchloride, polysuflone, and the like; see, e.g., column 11 of the patent.

In one embodiment, the polymeric material is bound to the anti-mitotic compound by one or more photosensitive linkers. The process of preparing and binding these photosensitive linkers is described in columns 8-9 of U.S. Pat. No. 5,470,307, wherein it is disclosed that: “The process of fabricating a catheter 10 having a desired therapeutic agent 20 connected thereto and then controllably and selectively releasing that therapeutic agent 20 at a remote site within a patient may be summarized in five steps. 1. Formation of Substrate. The substrate layer 16 is formed on or applied to the surface 14 of the catheter body 12, and subsequently or simultaneously prepared for coupling to the linker layer 18. This is accomplished by modifying the substrate layer 16 to expose or add groups such as carboxyls, amines, hydroxyls, or sulfhydryls. In some cases, this may be followed by customizing the substrate layer 16 with an extender 22 that will change the functionality, for example by adding a maleimide group that will accept a Michael's addition of a sulfhydryl at one end of a bifunctional photolytic linker 18. The extent of this derivitization is measured by adding group-specific probes (such as 1 pyrenyl diazomethane for carboxyls, 1 pyrene butyl hydrazine for amines, or Edman's reagent for sulfhydryls Molecular Probes, Inc. of Eugene, Oreg. or Pierce Chemical of Rockford, Ill.) or other fluorescent dyes that may be measured optically or by flow cytometry. The substrate layer 16 can be built up to increase its capacity by several methods, examples of which are discussed below.”

As is also dislosed in U.S. Pat. No. 5,470,307, “2. Selection of Photolytic Release Mechanism. A heterobifunctional photolytic linker 18 suitable for the selected therapeutic agent d20 and designed to couple readily to the functionality of the substrate layer 16 is prepared, and may be connected to the substrate layer 16. Alternately, the photolinker 18 may first be bonded to the therapeutic agent 20, with the combined complex of the therapeutic agent 20 and photolytic linker 18 together being connected to the substrate layer 16. 3. Selection of the Therapeutic Agent. Selection of the appropriate therapeutic agent 20 for a particular clinical application will depend upon the prevailing medical practice. One representative example described below for current use in PTCA and PTA procedures involves the amine terminal end of a twelve amino acid peptide analogue of hirudin being coupled to a chloro carbonyl group on the photolytic linker 18. Another representative example is provided below where the therapeutic agent 20 is a nucleotide such as an antisense oligodeoxynucleotide where a terminal phosphate is bonded by means of a diazoethane located on the photolytic linker 18. A third representative example involves the platelet inhibitor dipyridamole (persantin) that is attached through an alkyl hydroxyl by means of a diazo ethane on the photolytic linker 18. 4. Fabrication of the Linker-Agent Complex and Attachment to the Substrate. The photolytic linker 18 or the photolytic linker 18 with the therapeutic agent 20 attached are connected to the substrate layer 16 to complete the catheter 10. A representative example is a photolytic linker 18 having a sulfhydryl disposed on the non-photolytic end for attachment to the substrate layer 16, in which case the coupling will occur readily in a neutral buffer solution to a maleimide-modified substrate layer 16 on the catheter 10. Once the therapeutic agent 20 has been attached to the catheter 10, it is necessary that the catheter 10 be handled in a manner that prevents damage to the substrate layer 16, photolytic linker layer 18, and therapeutic agent 20, which may include subsequent sterilization, protection from ambient light, heat, moisture, and other environmental conditions that would adversely affect the operation or integrity of the drug-delivery catheter system 10 when used to accomplish a specific medical procedure on a patient.”

In the process of U.S. Pat. No. 5,470,307, the linker is preferably bound to the polymeric material through a modified functional group. The preparation of such modified functional groups is discussed at columns 10-13 of such patent, wherein it is disclosed that: “Most polymers including those discussed herein can be made of materials which have modifiable functional groups or can be treated to expose such groups. Polyamide (nylon) can be modified by acid treatment to produce exposed amines and carboxyls. Polyethylene terephthalate (PET, Dacron®) is a polyester and can be chemically treated to expose hydroxyls and carboxyls. Polystyrene has an exposed phenyl group that can be derivitized. Polyethylene and polypropylene (collectively referred to as polyolefins) have simple carbon backbones which can be derivitized by treatment with chromic and nitric acids to produce carboxyl functionality, photocoupling with suitably modified benzophenones, or by plasma grafting of selected monomers to produce the desired chemical functionality. For example, grafting of acrylic acid will produce a surface with a high concentration of carboxyl groups, whereas thiophene or 1,6 diaminocyclohexane will produce a surface containing sulfhydryls or amines, respectively. The surface functionality can be modified after grafting of a monomer by addition of other functional groups. For example, a carboxyl surface can be changed to an amine by coupling 1,6 diamino hexane, or to a sulfhydryl surface by coupling mercapto ethyl amine.”

As is also disclosed in U.S. Pat. No. 5,470,307, “Acrylic acid can be polymerized onto latex, polypropylene, polysulfone, and polyethylene terephthalate (PET) surfaces by plasma treatment. When measured by toluidine blue dye binding, these surfaces show intense modification. On polypropylene microporous surfaces modified by acrylic acid, as much as 50 nanomoles of dye binding per cm2 of external surface area can be found to represent carboxylated surface area. Protein can be linked to such surfaces using carbonyl diimidazole (CDI) in tetrahydrofuran as a coupling system, with a resultant concentration of one nanomole or more per cm2 of external surface. For a 50,000 Dalton protein, this corresponds to 50 μg per cm2, which is far above the concentration expected with simple plating on the surface. Such concentrations of a anti-mitotic compound 20 on the angioplasty (PTCA) balloon of a catheter 10, when released, would produce a high concentration of that therapeutic agent 20 at the site of an expanded coronary artery. However, plasmamodified surfaces are difficult to control and leave other oxygenated carbons that may cause undesired secondary reactions.”

As is also disclosed in U.S. Pat. No. 5,470,307, “In the case of balloon dilation catheters 10, creating a catheter body 12 capable of supporting a substrate layer 16 with enhanced surface area can be done by several means known to the art including altering conditions during balloon spinning, doping with appropriate monomers, applying secondary coatings such as polyethylene oxide hydrogel, branched polylysines, or one of the various Starburst.™. dendrimers offered by the Aldrich Chemical Company of Milwaukee, Wis.”

As is also disclosed in U.S. Pat. No. 5,470,307, “The most likely materials for the substrate layer 16 in the case of a dilation balloon catheter 10 or similar apparatus are shown in FIGS. 1a-1g, including synthetic or natural polymers such as polyamide, polyester, polyolefin (polypropylene or polyethylene), polyurethane, and latex. For solid support catheter bodies 12, usable plastics might include acrylamides, methacrylates, urethanes, polyvinylchloride, polysulfone, or other materials such as glass or quartz, which are all for the most part derivitizable.” In one embodiment, depicted in FIG. 1A, the photosensitive linker is bonded to a plastic container 12.

As is also disclosed in U.S. Pat. No. 5,470,307, “Referring to the polymers shown in FIGS. 1a-1g, polyamide (nylon) is treated with 3-5M hydrochloric acid to expose amines and carboxyl groups using conventional procedures developed for enzyme coupling to nylon tubing. A further description of this process may be obtained from Inman, D. J. and Hornby, W. E., The Iramobilization of Enzymes on Nylon Structures and their Use in Automated Analysis, Biochem. J. 129:255-262 (1972) and Daka, N. J. and Laidler, Flow kinetics of lactate dehydrogenase chemically attached to nylon tubing, K. J., Can. J. Biochem. 56:774-779 (1978). This process will release primary amines and carboxyls. The primary amine group can be used directly, or succinirnidyl 4 (p-maleimidophenyl) butyrate (SMBP) can be coupled to the amine function leaving free the maleimide to couple with a sulfhydryl on several of the photolytic linkers 18 described below and acting as an extender 22. If needed, the carboxyl released can also be converted to an amine by first protecting the amines with BOC groups and then coupling a diamine to the carboxyl by means of carbonyl diimidazole (CDI).” The polymeric material 14, and/or the container 12, may comprise or consist essentially of nylon.

As is also disclosed in U.S. Pat. No. 5,470,307, “Polyester (Dacron®) can be functionalized using 0.01N NaOH in 10% ethanol to release hydroxyl and carboxyl groups in the manner described by Blassberger, D. et al, Chemically Modified Polyesters as Supports for Enzyme Iramobilization: lsocyanide, Acylhydrazine, and Aminoaryl derivatives of Poly(ethylene Terephthalate), Biotechnol. and Bioeng. 20:309-315 (1978). A diamine is added directly to the etched surface using CDI and then reacted with SMBP to yield the same maleimide reacting group to accept the photolytic linker 18.” The polymeric material 14, and/or the container 12, may comprise or consist essentially of polyester.”

As is also disclosed in U.S. Pat. No. 5,470,307, “Polystyrene can be modified many ways, however perhaps the most useful process is chloromethylation, as originally described by Merrifield, R. B., Solid Phase Synthesis. I. The Synthesis of a Tetrapeptide, J. Am. Chem Soc. 85:2149-2154 (1963), and later discussed by Atherton, E. and Sheppard, R. C., Solid Phase Peptide Synthesis: A Practical Approach, pp. 13-23, (IRL Press 1989). The chlorine can be modified to an amine by reaction with anhydrous ammonia.” The polymeric material may be comprised of or consist essentially of polystyrene.

As is also disclosed in U.S. Pat. No. 5,470,307, “Polyolefins (polypropylene or polyethylene) require different approaches because they contain primarily a carbon backbone offering no native functional groups. One suitable approach is to add carboxyls to the surface by oxidizing with chromic acid followed by nitric acid as described by Ngo, T. T. et al., Kinetics of acetylcholinesterase immobilized on polyethylene tubing, Can. J. Biochem. 57:1200-1203 (1979). These carboxyls are then converted to amines by reacting successively with thionyl chloride and ethylene diamine. The surface is then reacted with SMBP to produce a maleimide that will react with the sulfhydryl on the photolytic linker 18.” The polymeric material may be comprised of or consist essentially of polyolefin material.

As is also disclosed in U.S. Pat. No. 5,470,307, “A more direct method is to react the polyolefin surfaces with benzophenone 4-maleimide as described by Odom, O. W. et al, Relaxation Time, Interthiol Distance, and Mechanism of Action of Ribosomal Protein S1, Arch. Biochem Biophys. 230:178-193 (1984), to produce the required group for the sulfhydryl addition to the photolytic linker 18. The benzophenone then links to the polyolefin through exposure to ultraviolet (uv) light. Other methods to derivitize the polyolefin surface include the use of radio frequency glow discharge (RFGD)—also known as plasma discharge—in several different manners to produce an in-depth coating to provide functional groups as well as increasing the effective surface area. Polyethylene oxide (PEO) can be crosslinked to the surface, or polyethylene glycol (PEG) can also be used and the mesh varied by the size of the PEO or PEG. This is discussed more fully by Sheu, M. S., et al., A glow discharge treatment to immobilize poly(ethylene oxide)/poly(propylene oxide) surfactants for wettable and non-fouling biomaterials, J. Adhes. Sci. Tech., 6:995-1009 (1992) and Yasuda, H., Plasma Polymerization, (Academic Press, Inc. 1985). Exposed hydroxyls can be activated by tresylation, also known as trifluoroethyl sulfonyl chloride activation, in the manner described by Nielson, K. and Mosbach, K., Tresyl Chloride-Activated Supports for Enzyme Immobilization (and related articles), Meth. Enzym., 135:65-170 (1987). The function can be converted to amines by addition of ethylene diamine or other aliphatic diamines, and then the usual addition of SMBP will give the required maleimide. Another suitable method is to use RFGD to polymerize acrylic acid or other monomers on the surface of the polyolefin. This surface consisting of carboxyls and other carbonyls is derivitizable with CDI and a diamine to give an amine surface which then can react with SMBP.”

Referring again to the process described in U.S. Pat. No. 5,470,307, photolytic linkers can be conjugated to the functional groups on substrate layers to form linker-agent complexes. As is disclosed in columns 13-14 of such patent, “Once a particular functionality for the substrate layer 16 has been determined, the appropriate strategy for coupling the photolytic linker 18 can be selected and employed. Several such strategies are set out in the examples which follow. As with selecting a method to expose a functional group on the surface 14 of the substrate layer 16, it is understood that selection of the appropriate strategy for coupling the photolytic linker 18 will depend upon various considerations including the chemical functionality of the substrate layer 16, the particular therapeutic agent 20 to be used, the chemical and physical factors affecting the rate and equilibrium of the particular photolytic release mechanism, the need to minimize any deleterious side-effects that might result (such as the production of antagonistic or harmful chemical biproducts, secondary chemical reactions with adjunct medical instruments including other portions of the catheter 10, unclean leaving groups or other impurities), and the solubility of the material used to fabricate the catheter body 12 or substrate layer 16 in various solvents. More limited strategies are available for the coupling of a 2-nitrophenyl photolytic linker 18. If the active site is 1-ethyl hydrazine used in most caging applications, then the complementary functionality on the therapeutic agent 20 will be a carboxyl, hydroxyl, or phosphate available on many pharmaceutical drugs. If a bromomethyl group is built into the photolytic linker 18, it can accept either a carboxyl or one of many other functional groups, or be converted to an amine which can then be further derivitized. In such a case, the leaving group might not be clean and care must be taken when adopting this strategy for a particular anti-mitotic compound20. Other strategies include building in an oxycarbonyl in the 1-ethyl position, which can form an urethane with an amine in the anti-mitotic compound20. In this case, the photolytic process evolves CO₂.”

Referring again to U.S. Pat. No. 5,470,307, after the photolytic linker construct has been prepared, it may be contacted with a coherent laser light source to release the therapeutic agent. Thus, as is disclosed in column 9 of U.S. Pat. No. 5,470,307, “use of a coherent laser light source 26 will be preferable in many applications because the use of one or more discrete wavelengths of light energy that can be tuned or adjusted to the particular photolytic reaction occurring in the photolytic linker 18 will necessitate only the minimum power (wattage) level necessary to accomplish a desired release of the anti-mitotic compound 20. As discussed above, coherent or laser light sources 26 are currently used in a variety of medical procedures including diagnostic and interventional treatment, and the wide availability of laser sources 26 and the potential for redundant use of the same laser source 26 in photolytic release of the therapeutic agent 20 as well as related procedures provides a significant advantage. In addition, multiple releases of different therapeutic agents 20 or multiple-step reactions can be accomplished using coherent light of different wavelengths, intermediate linkages to dye filters may be utilized to screen out or block transmission of light energy at unused or antagonistic wavelengths (particularly cytotoxic or cytogenic wavelengths), and secondary emitters may be utilized to optimize the light energy at the principle wavelength of the laser source 26. In other applications, it may be suitable to use a light source 26 such as a flash lamp operatively connected to the portion of the body 12 of the catheter 10 on which the substrate 16, photolytic linker layer 18, and anti-mitotic compound 20 are disposed. One example would be a mercury flash lamp capable of producing long-wave ultraviolet (uv) radiation within or across the 300-400 nanometer wavelength spectrum. When using either a coherent laser light source 26 or an alternate source 26 such as a flash lamp, it is generally preferred that the light energy be transmitted through at least a portion of the body 12 of the catheter 10 such that the light energy traverses a path through the substrate layer 16 to the photolytic linker layer 18 in order to maximize the proportion of light energy transmitted to the photolytic linker layer 18 and provide the greatest uniformity and reproducibility in the amount of light energy (photons) reaching the photolytic linker layer 18 from a specified direction and nature. Optimal uniformity and reproducibility in exposure of the photolyric linker layer 18 permits advanced techniques such as variable release of the anti-mitotic compound 20 dependent upon the controlled quantity of light energy incident on the substrate layer 16 and photolytic linker layer 18.”

As is also disclosed in U.S. Pat. No. 5,470,307, “The art pertaining to the transmission of light energy through fiber optic conduits 28 or other suitable transmission or production means to the remote biophysical site is extensively developed. For a fiber optic device, the fiber optic conduit 28 material must be selected to accommodate the wavelengths needed to achieve release of the anti-mitotic compound 20 which will for almost all applications be within the range of 280-400 nanometers. Suitable fiber optic materials, connections, and light energy sources 26 may be selected from those currently available and utilized within the biomedical field. While fiber optic conduit 28 materials may be selected to optimize transmission of light energy at certain selected wavelengths for desired application, the construction of a catheter 10 including fiber optic conduit 28 materials capable of adequate transmission throughout the range of the range of 280-400 nanometers is preferred, since this catheter 10 would be usable with the full compliment of photolytic release mechanisms and therapeutic agents 10. Fabrication of the catheter 10 will therefore depend more upon considerations involving the biomedical application or procedure by which the catheter 10 will be introduced or implanted in the patient, and any adjunct capabilities which the catheter 10 must possess.”

By way of yet further illustration, and referring to U.S. Pat. No. 5,599,352 (the entire disclosure of which is hereby incorporated by reference into this specification), the polymeric material can comprise fibrin. As is disclosed in column 4 of such patent, “The present invention provides a stent comprising fibrin. The term “fibrin” herein means the naturally occurring polymer of fibrinogen that arises during blood coagulation. Blood coagulation generally requires the participation of several plasma protein coagulation factors: factors XII, XI, IX, X, VIII, VII, V, XIII, prothrombin, and fibrinogen, in addition to tissue factor (factor II), kallikrein, high molecular weight kininogen, Ca+2, and phospholipid. The final event is the formation of an insoluble, cross-linked polymer, fibrin, generated by the action of thrombin on fibrinogen. Fibrinogen has three pairs of polypeptide chains (ALPHA 2—BETA 2—GAMMA 2) covalently linked by disulfide bonds with a total molecular weight of about 340,000. Fibrinogen is converted to fibrin through proteolysis by thrombin. An activation peptide, fibrinopeptide A (human) is cleaved from the amino-terminus of each ALPHA chain; fibrinopeptide B (human) from the amino-terminus of each BETA chain. The resulting monomer spontaneously polymerizes to a fibrin gel. Further stabilization of the fibrin polymer to an insoluble, mechanically strong form, requires cross-linking by factor XIII. Factor XIII is converted to XIIIa by thrombin in the presence of Ca+2. XIIIa cross-links the GAMMA chains of fibrin by transglutaminase activity, forming EPSILON-(GAMMA-glutamyl) lysine cross-links. The ALPHA chains of fibrin also may be secondarily cross-linked by transamidation.”

As is also disclosed in U.S. Pat. No. 5,599,352, “Since fibrin blood clots are naturally subject to fibrinolysis as part of the body's repair mechanism, implanted fibrin can be rapidly biodegraded. Plasminogen is a circulating plasma protein that is adsorbed onto the surface of the fibrin polymer. The adsorbed plasminogen is converted to plasmin by plasminogen activator released from the vascular endothelium. The plasmin will then break down the fibrin into a collection of soluble peptide fragments.”

As is also disclosed in U.S. Pat. No. 5,599,352, “Methods for making fibrin and forming it into implantable devices are well known as set forth in the following patents and published applications which are hereby incorporated by reference. In U.S. Pat. No. 4,548,736 issued to Muller et al., fibrin is clotted by contacting fibrinogen with a fibrinogen-coagulating protein such as thrombin, reptilase or ancrod. Preferably, the fibrin in the fibrin-containing stent of the present invention has Factor XIII and calcium present during clotting, as described in U.S. Pat. No. 3,523,807 issued to Gerendas, or as described in published European Patent Application 0366564, in order to improve the mechanical properties and biostability of the implanted device. Also preferably, the fibrinogen and thrombin used to make fibrin in the present invention are from the same animal or human species as that in which the stent of the present invention will be implanted in order to avoid cross-species immune reactions. The resulting fibrin can also be subjected to heat treatment at about 150° C. for 2 hours in order to reduce or eliminate antigenicity. In the Muller patent, the fibrin product is in the form of a fine fibrin film produced by casting the combined fibrinogen and thrombin in a film and then removing moisture from the film osmotically through a moisture permeable membrane. In the European Patent Application 0366564, a substrate (preferably having high porosity or high affinity for either thrombin or fibrinogen) is contacted with a fibrinogen solution and with a thrombin solution. The result is a fibrin layer formed by polymerization of fibrinogen on the surface of the device. Multiple layers of fibrin applied by this method could provide a fibrin layer of any desired thickness. Or, as in the Gerendas patent, the fibrin can first be clotted and then ground into a powder which is mixed with water and stamped into a desired shape in a heated mold. Increased stability can also be achieved in the shaped fibrin by contacting the fibrin with a fixing agent such as glutaraldehyde or formaldehyde. These and other methods known by those skilled in the art for making and forming fibrin may be used in the present invention.”

As is also disclosed in U.S. Pat. No. 5,599,352, “Preferably, the fibrinogen used to make the fibrin is a bacteria-free and virus-free fibrinogen such as that described in U.S. Pat. No. 4,540,573 to Neurath et al which is hereby incorporated by reference. The fibrinogen is used in solution with a concentration between about 10 and 50 mg/ml and with a pH of about 5.8-9.0 and with an ionic strength of about 0.05 to 0.45. The fibrinogen solution also typically contains proteins and enzymes such as albumin, fibronectin (0-300 μg per ml fibrinogen), Factor XIII (0-20 μg per ml fibrinogen), plasminogen (0-210 μg per ml fibrinogen), antiplasmin (0-61 μg per ml fibrinogen) and Antithrombin III (0-150 μg per ml fibrinogen). The thrombin solution added to make the fibrin is typically at a concentration of 1 to 120 NIH units/ml with a preferred concentration of calcium ions between about 0.02 and 0.2M.”

As is also disclosed in U.S. Pat. No. 5,599,352, “Polymeric materials can also be intermixed in a blend or co-polymer with the fibrin to produce a material with the desired properties of fibrin with improved structural strength. For example, the polyurethane material described in the article by Soldani et at., “Bioartificial Polymeric Materials Obtained from Blends of Synthetic Polymers with Fibrin and Collagen” International Journal of Artificial Organs, Vol. 14, No. 5, 1991, which is incorporated herein by reference, could be sprayed onto a suitable stent structure. Suitable polymers could also be biodegradable polymers such as polyphosphate ester, polyhydroxybutyrate valerate, polyhydroxybutyrate-co-hydroxyvalerate and the like . . . ” The polymeric material 14 may be, e.g., a blend of fibrin and another polymeric material.

As is also disclosed in U.S. Pat. No. 5,599,352, “The shape for the fibrin can be provided by molding processes. For example, the mixture can be formed into a stent having essentially the same shape as the stent shown in U.S. Pat. No. 4,886,062 issued to Wiktor. Unlike the method for making the stent disclosed in Wiktor which is wound from a wire, the stent made with fibrin can be directly molded into the desired open-ended tubular shape.”

As is also disclosed in U.S. Pat. No. 5,599,352, “In U.S. Pat. No. 4,548,736 issued to Muller et al., a dense fibrin composition is disclosed which can be a bioabsorbable matrix for delivery of drugs to a patient. Such a fibrin composition can also be used in the present invention by incorporating a drug or other therapeutic substance useful in diagnosis or treatment of body lumens to the fibrin provided on the stent. The drug, fibrin and stent can then be delivered to the portion of the body lumen to be treated where the drug may elute to affect the course of restenosis in surrounding luminal tissue. Examples of drugs that are thought to be useful in the treatment of restenosis are disclosed in published international patent application WO 91/12779 “Intraluminal Drug Eluting Prosthesis” which is incorporated herein by reference. Therefore, useful drugs for treatment of restenosis and drugs that can be incorporated in the fibrin and used in the present invention can include drugs such as anticoagulant drugs, antiplatelet drugs, antimetabolite drugs, anti-inflammatory drugs and antimitotic drugs. Further, other vasoreactive agents such as nitric oxide releasing agents could also be used. Such therapeutic substances can also be microencapsulated prior to their inclusion in the fibrin. The micro-capsules then control the rate at which the therapeutic substance is provided to the blood stream or the body lumen. This avoids the necessity for dehydrating the fibrin as set forth in Muller et al., since a dense fibrin structure would not be required to contain the therapeutic substance and limit the rate of delivery from the fibrin. For example, a suitable fibrin matrix for drug delivery can be made by adjusting the pH of the fibrinogen to below about pH 6.7 in a saline solution to prevent precipitation (e.g., NACl, CaCl, etc.), adding the microcapsules, treating the fibrinogen with thrombin and mechanically compressing the resulting fibrin into a thin film. The microcapsules which are suitable for use in this invention are well known. For example, the disclosures of U.S. Pat. Nos. 4,897,268, 4,675,189; 4,542,025; 4,530,840; 4,389,330; 4,622,244; 4,464,317; and 4,943,449 could be used and are incorporated herein by reference. Alternatively, in a method similar to that disclosed in U.S. Pat. No. 4,548,736 issued to Muller et al., a dense fibrin composition suitable for drug delivery can be made without the use of microcapsules by adding the drug directly to the fibrin followed by compression of the fibrin into a sufficiently dense matrix that a desired elution rate for the drug is achieved. In yet another method for incorporating drugs which allows the drug to elute at a controlled rate, a solution which includes a solvent, a polymer dissolved in the solvent and a therapeutic drug dispersed in the solvent is applied to the structural elements of the stent and then the solvent is evaporated. Fibrin can then be added over the coated structural elements in an adherent layer. The inclusion of a polymer in intimate contact with a drug on the underlying stent structure allows the drug to be retained on the stent in a resilient matrix during expansion of the stent and also slows the administration of drug following implantation. The method can be applied whether the stent has a metallic or polymeric surface. The method is also an extremely simple method since it can be applied by simply immersing the stent into the solution or by spraying the solution onto the stent. The amount of drug to be included on the stent can be readily controlled by applying multiple thin coats of the solution while allowing it to dry between coats. The overall coating should be thin enough so that it will not significantly increase the profile of the stent for intravascular delivery by catheter. It is therefore preferably less than about 0.002 inch thick and most preferably less than 0.001 inch thick. The adhesion of the coating and the rate at which the drug is delivered can be controlled by the selection of an appropriate bioabsorbable or biostable polymer and by the ratio of drug to polymer in the solution. By this method, drugs such as glucocorticoids (e.g. dexamethasone, betamethasone), heparin, hirudin, tocopherol, angiopeptin, aspirin, ACE inhibitors, growth factors, oligonucleotides, and, more generally, antiplatelet agents, anticoagulant agents, antimitotic agents, antioxidants, antimetabolite agents, and anti-inflammatory agents can be applied to a stent, retained on a stent during expansion of the stent and elute the drug at a controlled rate. The release rate can be further controlled by varying the ratio of drug to polymer in the multiple layers. For example, a higher drug-to-polymer ratio in the outer layers than in the inner layers would result in a higher early dose which would decrease over time. Examples of some suitable combinations of polymer, solvent and therapeutic substance are set forth in Table 1 below . . . .”

At column 7 of U.S. Pat. No. 5,599,352, some polymers that can be mixed with the fibrin are discussed. It is disclosed that: “The polymer used can be a bioabsorbable or biostable polymer. Suitable bioabsorbable polymers include poly(L-lactic acid), poly(lactide-co-glycolide) and poly(hydroxybutyrate-co-valerate). Suitable biostable polymers include silicones, polyurethanes, polyesters, vinyl homopolymers and copolymers, acrylate homopolymers and copolymers, polyethers and cellulosics. A typical ratio of drug to dissolved polymer in the solution can vary widely (e.g. in the range of about 10:1 to 1:100). The fibrin is applied by molding a polymerization mixture of fibrinogen and thrombin onto the composite as described herein.” The polymeric material 14 may be, e.g., a blend of fibrin and a bioabsorbable and/or biostable polymer.

By way of yet further illustration, and referring to U.S. Pat. No. 5,605,696, the polymeric material can be a multi-layered polymeric material, and/or a porous polymeric material. Thus, e.g., and as is disclosed in claim 25 of such patent, “A polymeric material containing a therapeutic drug for application to an intravascular stent for carrying and delivering said therapeutic drug within a blood vessel in which said intravascular stent is placed, comprising: a polymeric material having a thermal processing temperature no greater than about 100° C.; particles of a therapeutic drug incorporated in said polymeric material; and a porosigen uniformly dispersed in said polymeric material, said porosigen being selected from the group consisting of sodium chloride, lactose, sodium heparin, polyethylene glycol, copolymers of polyethylene oxide and polypropylene oxide, and mixtures thereof.” The “porsigen” is described at columns 4 and 5 of the patent, wherein it is disclosed that: “porosigen can also be incorporated in the drug loaded polymer by adding the porosigen to the polymer along with the therapeutic drug to form a porous, drug loaded polymeric membrane. A porosigen is defined herein for purposes of this application as any moiety, such as microgranules of sodium chloride, lactose, or sodium heparin, for example, which will dissolve or otherwise be degraded when immersed in body fluids to leave behind a porous network in the polymeric material. The pores left by such porosigens can typically be a large as 10 microns. The pores formed by porosigens such as polyethylene glycol (PEG), polyethylene oxide/polypropylene oxide (PEO/PPO) copolymers, for example, can also be smaller than one micron, although other similar materials which form phase separations from the continuous drug loaded polymeric matrix and can later be leached out by body fluids can also be suitable for forming pores smaller than one micron. While it is currently preferred to apply the polymeric material to the structure of a stent while the therapeutic drug and porosigen material are contained within the polymeric material, to allow the porosigen to be dissolved or degraded by body fluids when the stent is placed in a blood vessel, alternatively the porosigen can be dissolved and removed from the polymeric material to form pores in the polymeric material prior to placement of the polymeric material combined with the stent within a blood vessel. If desired, a rate-controlling membrane can also be applied over the drug loaded polymer, to limit the release rate of the therapeutic drug. Such a rate-controlling membrane can be useful for delivery of water soluble substances where a nonporous polymer film would completely prevent diffusion of the drug. The rate-controlling membrane can be added by applying a coating from a solution, or a lamination, as described previously. The rate-controlling membrane applied over the polymeric material can be formed to include a uniform dispersion of a porosigen in the rate-controlling membrane, and the porosigen in the rate-controlling membrane can be dissolved to leave pores in the rate-controlling membrane typically as large as 10 microns, or as small as 1 micron, for example, although the pores can also be smaller than 1 micron. The porosigen in the rate-controlling membrane can be, for example, sodium chloride, lactose, sodium heparin, polyethylene glycol, polyethylene oxide/polypropylene oxide copolymers, and mixtures thereof.” The polymeric material 14 may comprise a multiplicity of layers of polymeric material.

By way of yet further illustration, and referring to U.S. Pat. No. 5,700,286 (the entire disclosure of which is hereby incorporated by reference into this specification), the polymeric material may be either a thermoplastic or an elastomeric polymer. Thus, and referring to columns 5 and 6 of such patent, “The polymeric material is preferably selected from thermoplastic and elastomeric polymers. In one currently preferred embodiment the polymeric material can be a material available under the trade name “C-Flex” from Concept Polymer Technologies of Largo, Fla. In another currently preferred embodiment, the polymeric material can be ethylene vinyl acetate (EVA); and in yet another currently preferred embodiment, the polymeric material can be a material available under the trade name “BIOSPAN.” Other suitable polymeric materials include latexes, urethanes, polysiloxanes, and modified styrene-ethylene/butylene-styrene block copolymers (SEBS) and their associated families, as well as elastomeric, bioabsorbable, linear aliphatic polyesters. The polymeric material can typically have a thickness in the range of about 0.002 to about 0.020 inches, for example. The polymeric material is preferably bioabsorbable, and is preferably loaded or coated with a anti-mitotic compound or drug, including, but not limited to, antiplatelets, antithrombins, cytostatic and antiproliferative agents, for example, to reduce or prevent restenosis in the vessel being treated.”

By way of yet further illustration, and referring to U.S. Pat. No. 6,004,346 (the entire disclosure of which is hereby incorporated by reference into this specification), the polymeric material may be a bioabsorbable polymer. Thus, and referring to column 7 of such patent, “controlled release, via a bioabsorbable polymer, offers to maintain the drug level within the desired therapeutic range for the duration of the treatment. In the case of stents, the prosthesis materials will maintain vessel support for at least two weeks or until incorporated into the vessel wall even with bioabsorbable, biodegradable polymer constructions.”

As is also disclosed in U.S. Pat. No. 6,004,346, “Several polymeric compounds that are known to be bioabsorbable and hypothetically have the ability to be drug impregnated may be useful in prosthesis formation herein. These compounds include: poly-1-lactic acid/polyglycolic acid, polyanhydride, and polyphosphate ester. A brief description of each is given below.”

As is also disclosed in U.S. Pat. No. 6,004,346, “Poly-1-lactic acid/polyglycolic acid has been used for many years in the area of bioabsorbable sutures. It is currently available in many forms, i.e., crystals, fibers, blocks, plates, etc . . . ”

As is also disclosed in U.S. Pat. No. 6,004,346, “Another compound which could be used are the polyanhydrides. They are currently being used with several chemotherapy drugs for the treatment of cancerous tumors. These drugs are compounded into the polymer which is molded into a cube-like structure and surgically implanted at the tumor site . . . ”

As is also disclosed in U.S. Pat. No. 6,004,346, “The compound which is preferred is a polyphosphate ester. Polyphosphate ester is a compound such as that disclosed in U.S. Pat. Nos. 5,176,907; 5,194,581; and 5,656,765 issued to Leong which are incorporated herein by reference. Similar to the polyanhydrides, polyphoshate ester is being researched for the sole purpose of drug delivery. Unlike the polyanhydrides, the polyphosphate esters have high molecular weights (600,000 average), yielding attractive mechanical properties. This high molecular weight leads to transparency, and film and fiber properties. It has also been observed that the phosphorous-carbon-oxygen plasticizing effect, which lowers the glass transition temperature, makes the polymer desirable for fabrication.”

As is also disclosed in U.S. Pat. No. 6,004,346, “The basic structure of polyphosphate ester monomer is shown below where P corresponds to Phosphorous, O corresponds to Oxygen, and R and R1 are functional groups. Reaction with water leads to the breakdown of this compound into monomeric phosphates (phosphoric acid) and diols (see below). [Figure] It is the hydrolytic instability of the phosphorous ester bond which makes this polymer attractive for controlled drug release applications. A wide range of controllable degradation rates can be obtained by adjusting the hydrophobicities of the backbones of the polymers and yet assure biodegradability. he functional side groups allow for the chemical linkage of drug molecules to the polymer . . . he drug may also be incorporated into the backbone of the polymer.”

By way of further illustration, and referring to U.S. Pat. No. 6,120,536 (the entire disclosure of which is hereby incorporated by reference into this specification), the polymeric material may comprise a hydrophobic elastomeric material incorporating an amount of anti-mitotic compound therein for timed release. Some of these elastomeric materials are described at columns 5 and 6 of such patent, wherein it is disclosed that: “The elastomeric materials that form the stent coating underlayers should possess certain properties. Preferably the layers should be of suitable hydrophobic biostable elastomeric materials which do not degrade. Surface layer material should minimize tissue rejection and tissue inflammation and permit encapsulation by tissue adjacent the stent implantation site. Exposed material is designed to reduce clotting tendencies in blood contacted and the surface is preferably modified accordingly. Thus, underlayers of the above materials are preferably provided with a fluorosilicone outer coating layer which may or may not contain imbedded bioactive material, such as heparin. Alternatively, the outer coating may consist essentially of polyethylene glycol (PEG), polysaccharides, phospholipids, or combinations of the foregoing.”

As is also disclosed in U.S. Pat. No. 6,120,536, “Polymers generally suitable for the undercoats or underlayers include silicones (e.g., polysiloxanes and substituted polysiloxanes), polyurethanes, thermoplastic elastomers in general, ethylene vinyl acetate copolymers, polyolefin elastomers, polyamide elastomers, and EPDM rubbers. The above-referenced materials are considered hydrophobic with respect to the contemplated environment of the invention. Surface layer materials include fluorosilicones and polyethylene glycol (PEG), polysaccharides, phospholipids, and combinations of the foregoing.”

As is also disclosed in U.S. Pat. No. 6,120,536, “Various combinations of polymer coating materials can be coordinated with biologically active species of interest to produce desired effects when coated on stents to be implanted in accordance with the invention. Loadings of therapeutic materials may vary. The mechanism of incorporation of the biologically active species into the surface coating and egress mechanism depend both on the nature of the surface coating polymer and the material to be incorporated. The mechanism of release also depends on the mode of incorporation. The material may elute via interparticle paths or be administered via transport or diffusion through the encapsulating material itself.”

By way of yet further illustration, and referring to U.S. Pat. No. 6,159,488 (the entire disclosure of which is hereby incorporated by reference into this specification), the polymeric material may be a biopolymer that is non-degradable and is insoluble in biological mediums. Thus, and as is disclosed at column 8 of this patent, “The polymer carrier can be any pharmaceutically acceptable biopolymer that is non-degradable and insoluble in biological mediums, has good stability in a biological environment, has a good adherence to the selected stent, is flexible, and that can be applied as coating to the surface of a stent, either from an organic solvent, or by a melt process. The hydrophilicity or hydrophobicity of the polymer carrier will determine the release rate of halofuginone from the stent surface. The coating may include other antiproliferative agents, such as heparin, steroids and non-steroidal anti-inflammatory agents. To improve the blood compatibility of the coated stent, a hydrophilic coating such as hydromer-hydrophilic polyurethane can be applied. A material for delivering a biologically active compound comprising a solid carrier material having dissolved and/or dispersed therein at least two biologically active compounds, each of said at least two biologically active compounds having a biologically active nucleus which is common to each of the biologically active compounds, and the at least two biologically active compounds having maximum solubility levels in a single solvent which differ from each other by at least 10% by weight; wherein said solid carrier comprises a biocompatible polymeric material.”

By way of yet further illustration, and referring to claim 1 of U.S. Pat. No. 6,168,801 (the entire disclosure of which is hereby incorporated by reference into this specification), the polymeric material may comprise “A material for delivering a biologically active compound comprising a solid carrier material having dissolved and/or dispersed therein at least two biologically active compounds, each of said at least two biologically active compounds having a biologically active nucleus which is common to each of the biologically active compounds, and the at least two biologically active compounds having maximum solubility levels in a single solvent which differ from each other by at least 10% by weight; wherein said solid carrier comprises a biocompatible polymeric material.”

The device of U.S. Pat. No. 6,168,801 preferably comprises at least two forms of a biologically active ingredient in a single polymeric matrix. Thus, and as is disclosed at column 6 of the patent, “It is contemplated in the practice of the present invention that the combination of the at least two forms of the biologically active ingredient or medically active ingredient in at least a single polymeric carrier can provide release of the active ingredient nucleus common to the at least two forms. The release of the active nucleus can be accomplished by, for example, enzymatic hydrolysis of the forms upon release from the carrier device. Further, the combination of the at least two forms of the biologically active ingredient or medically active ingredient in at least a single polymeric carrier can provide net active ingredient release characterized by the at least simple combination of the two matrix forms described above. This point is illustrated in FIG. 1 which compares the in vitro release of dexamethasone from matrices containing various fractions of two forms of the synthetic steroid dexamethasone, dexamethasone sodium phosphate (DSP; hydrophilic) and dexamethasone acetate (DA; hydrophobic). It is easy to see from these results that the release of dexamethasone acetate (specifically, 100% DA) is slower than all other matrices tested containing some degree or loading of dexamethasone sodium phosphate (hydrophilic). Still further, the resulting active ingredient release from the combined form matrix should be at least more rapid in the early stages of release than the slow single active ingredient component alone. Further still, the cumulative active ingredient release from the combined form matrix should be at least greater in the chronic stages than the fast single active ingredient component. Once again from FIG. 1, the two test matrices containing the greatest amount of dexamethasone sodium phosphate (specifically, 100% DSP, and 75% DSP/25% DA) began to slow in release as pointed out at points “A” and “B”. And further still, the optimal therapeutic release can be designed through appropriate combination of the at least two active biological or medical ingredients in the polymeric carrier material. If as in this example, rapid initial release as well as continuous long term release is desired to achieve a therapeutic goal, the matrix composed of 50% DSP/50% DA would be selected.”

By way of yet further illustration, and referring to claim 1 of U.S. Pat. No. 6,395,300 (the entire disclosure of which is hereby incorporated by reference into this specification), the polymeric material may be a porous polymeric matrix made by a process comprising the steps of: “a) dissolving a drug in a volatile organic solvent to form a drug solution, (b) combining at least one volatile pore forming agent with the volatile organic drug solution to form an emulsion, suspension, or second solution, and (c) removing the volatile organic solvent and volatile pore forming agent from the emulsion, suspension, or second solution to yield the porous matrix comprising drug, wherein the porous matrix comprising drug has a tap density of less than or equal to 1.0 g/mL or a total surface area of greater than or equal to 0.2 m2/g.”

The anti-mitotic compound may be derived from an anti-microtuble agent. As is disclosed in U.S. Pat. No. 6,689,803 (at columns 5-6), representative anti-microtubule agents include, e.g., “ . . . taxanes (e.g., paclitaxel and docetaxel), campothecin, eleutherobin, sarcodictyins, epothilones A and B, discodermolide, deuterium oxide (D2 O), hexylene glycol (2-methyl-2,4-pentanediol), tubercidin (7-deazaadenosine), LY290181 (2-amino-4-(3-pyridyl)-4H-naphtho(1,2-b)pyran-3-cardonitrile), aluminum fluoride, ethylene glycol bis-(succinimidylsuccinate), glycine ethyl ester, nocodazole, cytochalasin B, colchicine, colcemid, podophyllotoxin, benomyl, oryzalin, majusculamide C, demecolcine, methyl-2-benzimidazolecarbamate (MBC), LY195448, subtilisin, 1069C85, steganacin, combretastatin, curacin, estradiol, 2-methoxyestradiol, flavanol, rotenone, griseofulvin, vinca alkaloids, including vinblastine and vincristine, maytansinoids and ansamitocins, rhizoxin, phomopsin A, ustiloxins, dolastatin 10, dolastatin 15, halichondrins and halistatins, spongistatins, cryptophycins, rhazinilam, betaine, taurine, isethionate, HO-221, adociasulfate-2, estramustine, monoclonal anti-idiotypic antibodies, microtubule assembly promoting protein (taxol-like protein, TALP), cell swelling induced by hypotonic (190 mosmol/L) conditions, insulin (100 nmol/L) or glutamine (10 mmol/L), dynein binding, gibberelin, XCHO1 (kinesin-like protein), lysophosphatidic acid, lithium ion, plant cell wall components (e.g., poly-L-lysine and extensin), glycerol buffers, Triton X-100 microtubule stabilizing buffer, microtubule associated proteins (e.g., MAP2, MAP4, tau, big tau, ensconsin, elongation factor-1-alpha (EF-1.alpha.) and E-MAP-115), cellular entities (e.g., histone H1, myelin basic protein and kinetochores), endogenous microtubular structures (e.g., axonemal structures, plugs and GTP caps), stable tubule only polypeptide (e.g., STOP 145 and STOP220) and tension from mitotic forces, as well as any analogues and derivatives of any of the above. Within other embodiments, the anti-microtubule agent is formulated to further comprise a polymer.”

The term “anti-micrtubule,” as used in this specification (and in the specification of U.S. Pat. No. 6,689,803), refers to any “ . . . protein, peptide, chemical, or other molecule which impairs the function of microtubules, for example, through the prevention or stabilization of polymerization. A wide variety of methods may be utilized to determine the anti-microtubule activity of a particular compound, including for example, assays described by Smith et al. (Cancer Lett 79(2):213-219, 1994) and Mooberry et al., (Cancer Lett. 96(2):261-266, 1995);” see, e.g., lines 13-21 of column 14 of U.S. Pat. No. 6,689,803.

An extensive listing of anti-microtubule agents is provided in columns 14, 15, 16, and 17 of U.S. Pat. No. 6,689,803; and one or more of them may be disposed within the polymeric material together with and/or instead of the anti-mitotic compound of this invention. In one embodiment, these prior art anti-microtubule agents are made magnetic in accordance with the process described earlier in this specification.

These prior art anti-microtubule agents, which may be used to prepare the anti-mitotic compounds of this invention, include “ . . . taxanes (e.g., paclitaxel (discussed in more detail below) and docetaxel) (Schiff et al., Nature 277: 665-667, 1979; Long and Fairchild, Cancer Research 54: 4355-4361, 1994; Ringel and Horwitz, J. Natl. Cancer Inst. 83(4): 288-291, 1991; Pazdur et al., Cancer Treat. Rev. 19(4): 351-386, 1993), campothecin, eleutherobin (e.g., U.S. Pat. No. 5,473,057), sarcodictyins (including sarcodictyin A), epothilones A and B (Bollag et al., Cancer Research 55: 2325-2333, 1995), discodermolide (ter Haar et al., Biochemistry 35: 243-250, 1996), deuterium oxide (D2 O) (James and Lefebvre, Genetics 130(2): 305-314, 1992; Sollott et al., J. Clin. Invest. 95: 1869-1876, 1995), hexylene glycol (2-methyl-2,4-pentanediol) (Oka et al., Cell Struct. Funct. 16(2): 125-134, 1991), tubercidin (7-deazaadenosine) (Mooberry et al., Cancer Lett. 96(2): 261-266, 1995), LY290181 (2-amino-4-(3-pyridyl)-4H-naphtho(1,2-b)pyran-3-cardonitrile) (Panda et al., J. Biol. Chem. 272(12): 7681-7687, 1997; Wood et al., Mol. Pharmacol. 52(3): 437-444, 1997), aluminum fluoride (Song et al., J. Cell. Sci. Suppl. 14: 147-150, 1991), ethylene glycol bis-(succinimidylsuccinate) (Caplow and Shanks, J. Biol. Chem. 265(15): 8935-8941, 1990), glycine ethyl ester (Mejillano et al., Biochemistry 31(13): 3478-3483, 1992), nocodazole (Ding et al., J. Exp. Med. 171(3): 715-727, 1990; Dotti et al., J. Cell Sci. Suppl. 15: 75-84, 1991; Oka et al., Cell Struct. Funct. 16(2): 125-134, 1991; Weimer et al., J. Cell. Biol. 136(1), 71-80, 1997), cytochalasin B (Illinger et al., Biol. Cell 73(2-3): 131-138, 1991), colchicine and CI 980 (Allen et al., Am. J. Physiol. 261(4 Pt. 1): L315-L321, 1991; Ding et al., J. Exp. Med. 171(3): 715-727, 1990; Gonzalez et al., Exp. Cell. Res. 192(1): 10-15, 1991; Stargell et al., Mol. Cell. Biol. 12(4): 1443-1450, 1992; Garcia et al., Antican. Drugs 6(4): 533-544, 1995), colcernid (Barlow et al., Cell. Motil. Cytoskeleton 19(1): 9-17, 1991; Meschini et al., J. Microsc. 176(Pt. 3): 204-210, 1994; Oka et al., Cell Struct. Funct. 16(2): 125-134, 1991), podophyllotoxin (Ding et al., J. Exp. Med. 171(3): 715-727, 1990), benomyl (Hardwick et al., J. Cell. Biol. 131(3): 709-720, 1995; Shero et al., Genes Dev. 5(4): 549-560, 1991), oryzalin (Stargell et al., Mol. Cell. Biol. 12(4): 1443-1450, 1992), majusculamide C (Moore, J. Ind. Microbiol. 16(2): 134-143, 1996), demecolcine (Van Dolah and Ramsdell, J. Cell. Physiol. 166(1): 49-56, 1996; Wiemer et al., J. Cell. Biol. 136(1): 71-80, 1997), methyl-2-benzimidazolecarbamate (MBC) (Brown et al., J. Cell. Biol. 123(2): 387-403, 1993), LY195448 (Barlow & Cabral, Cell Motil. Cytoskel. 19: 9-17, 1991), subtilisin (Saoudi et al., J. Cell Sci. 108: 357-367, 1995), 1069C85 (Raynaud et al., Cancer Chemother. Pharmacol. 35: 169-173, 1994), steganacin (Hamel, Med. Res. Rev. 16(2): 207-231, 1996), combretastatins (Hamel, Med. Res. Rev. 16(2): 207-231, 1996), curacins (Hamel, Med. Res. Rev. 16(2): 207-231, 1996), estradiol (Aizu-Yokata et al., Carcinogen. 15(9): 1875-1879, 1994), 2-methoxyestradiol (Hamel, Med. Res. Rev. 16(2): 207-231, 1996), flavanols (Hamel, Med. Res. Rev. 16(2): 207-231, 1996), rotenone (Hamel, Med. Res. Rev. 16(2): 207-231, 1996), griseofulvin (Hamel, Med. Res. Rev. 16(2): 207-231; 1996), vinca alkaloids, including vinblastine and vincristine (Ding et al., J. Exp. Med. 171(3): 715-727, 1990; Dirket al., Neurochem. Res. 15(11): 1135-1139, 1990; Hamel, Med. Res. Rev. 16(2): 207-231, 1996; Illinger et al., Biol. Cell 73(2-3): 131-138, 1991; Wiemer et al., J. Cell. Biol. 136(1): 71-80, 1997), maytansinoids and ansamitocins (Hamel, Med. Res. Rev. 16(2): 207-231, 1996), rhizoxin (Hamel, Med. Res. Rev. 16(2): 207-231, 1996), phomopsin A (Hamel, Med. Res. Rev. 16(2): 207-231, 1996), ustiloxins (Hamel, Med. Res. Rev. 16(2): 207-231, 1996), dolastatin 10 (Hamel, Med Res. Rev. 16(2): 207-231, 1996), dolastatin 15 (Hamel, Med. Res. Rev. 16(2): 207-231, 1996), halichondrins and halistatins (Hamel, Med. Res. Rev. 16(2): 207-231, 1996), spongistatins (Hamel, Med. Res. Rev. 16(2): 207-231, 1996), cryptophycins (Hamel, Med. Res. Rev. 16(2): 207-231, 1996), rhazinilam (Hamel, Med. Res. Rev. 16(2): 207-231, 1996), betaine (Hashimoto et al., Zool. Sci. 1: 195-204, 1984), taurine (Hashimoto et al., Zool. Sci. 1: 195-204, 1984), isethionate (Hashimoto et al., Zool. Sci. 1: 195-204, 1984), HO-221 (Ando et al., Cancer Chemother. Pharmacol. 37: 63-69, 1995), adociasulfate-2 (Sakowicz et al., Science 280: 292-295, 1998), estramustine (Panda et al., Proc. Natl. Acad. Sci. USA 94: 10560-10564, 1997), monoclonal anti-idiotypic antibodies (Leu et al., Proc. Natl. Acad. Sci. USA 91(22): 10690-10694, 1994), microtubule assembly promoting protein (taxol-like protein, TALP) (Hwang et al., Biochem. Biophys. Res. Commun. 208(3): 1174-1180, 1995), cell swelling induced by hypotonic (190 mosmol/L) conditions, insulin (100 nmol/L) or glutamine (10 mmol/L) (Haussinger et al., Biochem. Cell. Biol. 72(1-2): 12-19, 1994), dynein binding (Ohba et al., Biochim. Biophys. Acta 1158(3): 323-332, 1993), gibberelin (Mita and Shibaoka, Protoplasma 119(1/2): 100-109, 1984), XCHO1 kinesin-like protein) (Yonetani et al., Mol. Biol. Cell 7(suppl): 211A, 1996), lysophosphatidic acid (Cook et al., Mol. Biol. Cell 6(suppl): 260A, 1995), lithium ion (Bhattacharyya and Wolff, Biochem. Biophys. Res. Commun. 73(2): 383-390, 1976), plant cell wall components (e.g., poly-L-lysine and extensin) (Akashi et al., Planta 182(3): 363-369, 1990), glycerol buffers (Schilstra et al., Biochem. J. 277(Pt. 3): 839-847, 1991; Farrell and Keates, Biochem. Cell. Biol. 68(11): 1256-1261, 1990; Lopez et al., J. Cell. Biochem. 43(3): 281-291, 1990), Triton X-100 microtubule stabilizing buffer (Brown et al., J. Cell Sci. 104(Pt. 2): 339-352, 1993; Safiejko-Mroczka and Bell, J. Histochem. Cytochem. 44(6): 641-656, 1996), microtubule associated proteins (e.g., MAP2, MAP4, tau, big tau, ensconsin, elongation factor-1-alpha EF-1.alpha.) and E-MAP-115) (Burgess et al., Cell Motil. Cytoskeleton 20(4): 289-300, 1991; Saoudi et al., J. Cell. Sci. 108(Pt. 1): 357-367, 1995; Bulinski and Bossler, J. Cell. Sci. 107(Pt. 10): 2839-2849, 1994; Ookata et al., J. Cell Biol. 128(5): 849-862, 1995; Boyne et al., J. Comp. Neurol. 358(2): 279-293, 1995; Ferreira and Caceres, J. Neurosci. 11(2): 392400, 1991; Thurston et al., Chromosoma 105(1): 20-30, 1996; Wang et al., Brain Res. Mol. Brain Res. 38(2): 200-208, 1996; Moore and Cyr, Mol. Biol. Cell 7(suppl): 221-A, 1996; Masson and Kreis, J. Cell Biol. 123(2), 357-371, 1993), cellular entities (e.g. histone H1, myelin basic protein and kinetochores) (Saoudi et al., J. Cell. Sci. 108(Pt. 1): 357-367, 1995; Simerly et al., J. Cell Biol. 111(4): 1491-1504, 1990), endogenous microtubular structures (e.g., axonemal structures, plugs and GTP caps) (Dye et al., Cell Motil. Cytoskeleton 21(3): 171-186, 1992; Azhar and Murphy, Cell Motil. Cytoskeleton 15(3): 156-161, 1990; Walker et al., J. Cell Biol. 114(1): 73-81, 1991; Drechsel and Kirschner, Curr. Biol. 4(12): 1053-1061, 1994), stable tubule only polypeptide (e.g., STOP145 and STOP220) (Pirollet et al., Biochim. Biophys. Acta 1160(1): 113-119, 1992; Pirollet et al., Biochemistry 31(37): 8849-8855, 1992; Bosc et al., Proc. Natl. Acad. Sci. USA 93(5): 2125-2130, 1996; Margolis et al., EMBO J. 9(12): 4095-4102, 1990) and tension from mitotic forces (Nicklas and Ward, J. Cell Biol. 126(5): 1241-1253, 1994), as well as any analogues and derivatives of any of the above. Such compounds can act by either depolymerizing microtubules (e.g., colchicine and vinblastine), or by stabilizing microtubule formation (e.g., paclitaxel).”

U.S. Pat. No. 6,689,803 also discloses (at columns 16 and 17 that, “Within one preferred embodiment of the invention, the therapeutic agent is is paclitaxel, a compound which disrupts microtubule formation by binding to tubulin to form abnormal mitotic spindles. Briefly, paclitaxel is a highly derivatized diterpenoid (Wani et al., J. Am. Chem. Soc. 93:2325, 1971) which has been obtained from the harvested and dried bark of Taxus brevifolia (Pacific Yew) and Taxomyces Andreanae and Endophytic Fungus of the Pacific Yew (Stierle et al., Science 60:214-216,-1993). “Paclitaxel” (which should be understood herein to include prodrugs, analogues and derivatives such as, for example, TAXOL®, TAXOTERE®, Docetaxel, 10-desacetyl analogues of paclitaxel and 3′N-desbenzoyl-3′N-t-butoxy carbonyl analogues of paclitaxel) may be readily prepared utilizing techniques known to those skilled in the art (see e.g., Schiff et al., Nature 277:665-667, 1979; Long and Fairchild, Cancer Research 54:4355-4361, 1994; Ringel and Horwitz, J. Natl. Cancer Inst. 83(4):288-291, 1991; Pazdur et al., Cancer Treat. Rev. 19(4):351-386, 1993; WO 94/07882; WO 94/07881; WO 94/07880; WO 94/07876; WO 93/23555; WO 93/10076; WO94/00156; WO 93/24476; EP 590267; WO 94/20089; U.S. Pat. Nos. 5,294,637; 5,283,253; 5,279,949; 5,274,137; 5,202,448; 5,200,534; 5,229,529; 5,254,580; 5,412,092; 5,395,850; 5,380,751; 5,350,866; 4,857,653; 5,272,171; 5,411,984; 5,248,796; 5,248,796; 5,422,364; 5,300,638; 5,294,637; 5,362,831; 5,440,056; 4,814,470; 5,278,324; 5,352,805; 5,411,984; 5,059,699; 4,942,184; Tetrahedron Letters 35(52):9709-9712, 1994; J. Med. Chem. 35:4230-4237, 1992; J. Med. Chem. 34:992-998, 1991; J. Natural Prod. 57(10):1404-1410, 1994; J. Natural Prod. 57(11):1580-1583, 1994; J. Am. Chem. Soc. 110:6558-6560, 1988), or obtained from a variety of commercial sources, including for example, Sigma Chemical Co., St. Louis, Mo. (T7402—from Taxus brevifolia).”

As is also disclosed in U.S. Pat. No. 6,689,893, “Representative examples of such paclitaxel derivatives or analogues include 7-deoxy-docetaxol, 7,8-cyclopropataxanes, N-substituted 2-azetidones, 6,7-epoxy paclitaxels, 6,7-modified paclitaxels, 10-desacetoxytaxol, 10-deacetyltaxol (from 10-deacetylbaccatin III), phosphonooxy and carbonate derivatives of taxol, taxol 2′,7-di(sodium 1,2-benzenedicarboxylate, 10-desacetoxy-11,12-dihydrotaxol-10,12(18)-diene derivatives, 10-desacetoxytaxol, Protaxol(2′- and/or 7-O-ester derivatives), (2′- and/or 7-O-carbonate derivatives), asymmetric synthesis of taxol side chain, fluoro taxols, 9-deoxotaxane, (13-acetyl-9-deoxobaccatine III, 9-deoxotaxol, 7-deoxy-9-deoxotaxol, 10-desacetoxy-7-deoxy-9-deoxotaxol, Derivatives containing hydrogen or acetyl group and a hydroxy and tert-butoxycarbonylamino, sulfonated 2′-acryloyltaxol and sulfonated 2′-O-acyl acid taxol derivatives, succinyltaxol, 2′-.gamma.-aminobutyryltaxol formate, 2′-acetyl taxol, 7-acetyl taxol, 7-glycine carbamate taxol, 2′-OH-7-PEG(5000)carbamate taxol, 2′-benzoyl and 2′,7-dibenzoyl taxol derivatives, other prodrugs (2′-acetyl taxol; 2′,7-diacetyltaxol; 2′succinyltaxol; 2′-(beta-alanyl)-taxol); 2′gamma-aminobutyryltaxol formate; ethylene glycol derivatives of 2′-succinyltaxol; 2′-glutaryltaxol; 2′-(N,N-dimethylglycyl)taxol; 2′-(2-(N,N-dimethylamino)propionyl)taxol; 2′orthocarboxybenzoyl taxol; 2′aliphatic carboxylic acid derivatives of taxol, Prodrugs {2′(N,N-diethylaminopropionyl)taxol, 2′(N,N-dimethylglycyl)taxol, 7(N,N-dimethylglycyl)taxol, 2′,7-di-(N,N-dimethylglycyl)taxol, 7(N,N-diethylaminopropionyl)taxol, 2′,7-di(N,N-diethylaminopropionyl)taxol, 2′-(L-glycyl)taxol, 7-(L-glycyl)taxol, 2′,7-di(L-glycyl)taxol, 2′-(L-alanyl)taxol, 7-(L-alanyl)taxol, 2′,7-di(L-alanyl)taxol, 2′-(L-leucyl)taxol, 7-(L-leucyl)taxol, 2′,7-di(L-leucyl)taxol, 2′-(L-isoleucyl)taxol, 7-(L-isoleucyl)taxol, 2′,7-di(L-isoleucyl)taxol, 2′-(L-valyl)taxol, 7-(L-valyl)taxol, 2′,7-di(L-valyl)taxol, 2′-(L-phenylalanyl)taxol, 7-(L-phenylalanyl)taxol, 2′,7-di(L-phenylalanyl)taxol, 2′-(L-prolyl)taxol, 7-(L-prolyl)taxol, 2′,7-di(L-prolyl)taxol, 2′-(L-lysyl)taxol, 7-(L-lysyl)taxol, 2′,7-di(L-lysyl)taxol, 2′-(L-glutamyl)taxol, 7-(L-glutamyl)taxol, 2′,7-di(L-glutamyl)taxol, 2′-(L-arginyl)taxol, 7-(L-arginyl)taxol, 2′,7-di(L-arginyl)taxol}, Taxol analogs with modified phenylisoserine side chains, taxotere, (N-debenzoyl-N-tert-(butoxycaronyl)-10-deacetyltaxol, and taxanes (e.g., baccatin III, cephalomannine, 10-deacetylbaccatin III, brevifoliol, yunantaxusin and taxusin).”

At columns 17, 18, 19, and 20 of U.S. Pat. No. 6,689,803, several “polymeric carriers” are described. One or more of these “polymeric carriers” may be used as the polymeric material. Thus, and referring to columns 17-20 of such United States patent, “ . . . a wide variety of polymeric carriers may be utilized to contain and/or deliver one or more of the therapeutic agents discussed above, including for example both biodegradable and non-biodegradable compositions. Representative examples of biodegradable compositions include albumin, collagen, gelatin, hyaluronic acid, starch, cellulose (methylcellulose, hydroxypropylcellulose, hydroxypropylmethylcellulose, hydroxyethylcellulose, carboxymethylcellulose, cellulose acetate phthalate, cellulose acetate succinate, hydroxypropylmethylcellulose phthalate), casein, dextrans, polysaccharides, fibrinogen, poly(D,L lactide), poly(D,L-lactide-co-glycolide), poly(glycolide), poly(hydroxybutyrate), poly(alkylcarbonate) and poly(orthoesters), polyesters, poly(hydroxyvaleric acid), polydioxanone, poly(ethylene terephthalate), poly(malic acid), poly(tartronic acid), polyanhydrides, polyphosphazenes, poly(amino acids) and their copolymers (see generally, Illum, L., Davids, S. S. (eds.) “Polymers in Controlled Drug Delivery” Wright, Bristol, 1987; Arshady, J. Controlled Release 17:1-22, 1991; Pitt, Int. J. Phar. 59:173-196, 1990; Holland et al., J. Controlled Release 4:155-0180, 1986). Representative examples of nondegradable polymers include poly(ethylene-vinyl acetate) (“EVA”) copolymers, silicone rubber, acrylic polymers (polyacrylic acid, polymethylacrylic acid, polymethylmethacrylate, polyalkylcynoacrylate), polyethylene, polyproplene, polyamides (nylon 6,6), polyurethane, poly(ester urethanes), poly(ether urethanes), poly(ester-urea), polyethers (poly(ethylene oxide), poly(propylene oxide), Pluronics and poly(tetramethylene glycol)), silicone rubbers and vinyl polymers (polyvinylpyrrolidone, poly(vinyl alcohol), poly(vinyl acetate phthalate). Polymers may also be developed which are either anionic (e.g. alginate, carrageenin, carboxymethyl cellulose and poly(acrylic acid), or cationic (e.g., chitosan, poly-L-lysine, polyethylenimine, and poly (allyl amine)) (see generally, Dunn et al., J. Applied Polymer Sci. 50:353-365, 1993; Cascone et al., J. Materials Sci.: Materials in Medicine 5:770-774, 1994; Shiraishi et al., Biol. Pharm. Bull. 16(11):1164-1168, 1993; Thacharodi and Rao, Int'l J. Pharm. 120:115-118, 1995; Miyazaki et al., Int'l J. Pharm. 118:257-263, 1995). Particularly preferred polymeric carriers include poly(ethylenevinyl acetate), poly (D,L-lactic acid) oligomers and polymers, poly (L-lactic acid) oligomers and polymers, poly (glycolic acid), copolymers of lactic acid and glycolic acid, poly (caprolactone), poly (valerolactone), polyanhydrides, copolymers of poly (caprolactone) or poly (lactic acid) with a polyethylene glycol (e.g., MePEG), and blends thereof.”

As is also disclosed in U.S. Pat. No. 6,689,893, “Polymeric carriers can be fashioned in a variety of forms, with desired release characteristics and/or with specific desired properties. For example, polymeric carriers may be fashioned to release a anti-mitotic compound upon exposure to a specific triggering event such as pH (see e.g., Heller et al., “Chemically Self-Regulated Drug Delivery Systems,” in Polymers in Medicine III, Elsevier Science Publishers B. V., Amsterdam, 1988, pp. 175-188; Kang et al., J. Applied Polymer Sci. 48:343-354, 1993; Dong et al., J. Controlled Release 19:171-178, 1992; Dong and Hoffmnan, J. Controlled Release 15:141-152, 1991; Kim et al., J. Controlled Release 28:143-152, 1994; Cornejo-Bravo et al., J. Controlled Release 33:223-229, 1995; Wu and Lee, Pharm. Res. 10(10):1544-1547, 1993; Serres et al., Pharm. Res. 13(2):196-201, 1996; Peppas, “Fundamentals of pH— and Temperature-Sensitive Delivery Systems,” in Gurny et al. (eds.), Pulsatile Drug Delivery, Wissenschaftliche Verlagsgesellschaft mbH, Stuttgart, 1993, pp. 41-55; Doelker, “Cellulose Derivatives,” 1993, in Peppas and Langer (eds.), Biopolymers I, Springer-Verlag, Berlin). Representative examples of pH-sensitive polymers include poly(acrylic acid) and its derivatives (including for example, homopolymers such as poly(aminocarboxylic acid); poly(acrylic acid); poly(methyl acrylic acid), copolymers of such homopolymers, and copolymers of poly(acrylic acid) and acrylmonomers such as those discussed above. Other pH sensitive polymers include polysaccharides such as cellulose acetate phthalate; hydroxypropylmethylcellulose phthalate; hydroxypropylmethylcellulose acetate succinate; cellulose acetate trimellilate; and chitosan. Yet other pH sensitive polymers include any mixture of a pH sensitive polymer and a water soluble polymer.”

As is also disclosed in U.S. Pat. No. 6,689,893, “Likewise, polymeric carriers can be fashioned which are temperature sensitive (see e.g., Chen et al., “Novel Hydrogels of a Temperature-Sensitive Pluronic Grafted to a Bioadhesive Polyacrylic Acid Backbone for Vaginal Drug Delivery,” in Proceed. Intern. Symp. Control. Rel. Bioact. Mater. 22:167-168, Controlled Release Society, Inc., 1995; Okano, “Molecular Design of Stimuli-Responsive Hydrogels for Temporal Controlled Drug Delivery,” in Proceed. Intern. Symp. Control. Rel. Bioact. Mater. 22:111-112, Controlled Release Society, Inc., 1995; Johnston et al., Pharm. Res. 9(3):425-433, 1992; Tung, Int'l J. Pharm. 107:85-90, 1994; Harsh and Gehrke, J. Controlled Release 17:175-186, 1991; Bae et al., Pharm. Res. 8(4):531-537, 1991; Dinarvand and D'Emanuele, J. Controlled Release 36:221-227, 1995; Yu and Grainger, “Novel Thermo-sensitive Amphiphilic Gels: Poly N-isopropylacrylamide-co-sodium acrylate-co-n-N-alkylacrylamide Network Synthesis and Physicochemical Characterization,” Dept. of Chemical & Biological Sci., Oregon Graduate Institute of Science & Technology, Beaverton, Oreg., pp. 820-821; Zhou and Smid, “Physical Hydrogels of Associative Star Polymers,” Polymer Research Institute, Dept. of Chemistry, College of Environmental Science and Forestry, State Univ. of New York, Syracuse, N.Y., pp. 822-823; Hoffman et al., “Characterizing Pore Sizes and Water ‘Structure’ in Stimuli-Responsive Hydrogels,” Center for Bioengineering, Univ. of Washington, Seattle, Wash., p. 828; Yu and Grainger, “Thermo-sensitive Swelling Behavior in Crosslinked N-isopropylacrylamide Networks: Cationic, Anionic and Ampholytic Hydrogels,” Dept. of Chemical & Biological Sci., Oregon Graduate Institute of Science & Technology, Beaverton, Oreg., pp. 829-830; Kim et al., Pharm. Res. 9(3):283-290, 1992; Bae et al., Pharm. Res. 8(5):624-628, 1991; Kono et al., J. Controlled Release 30:69-75, 1994; Yoshida et al., J. Controlled Release 32:97-102. 1994; Okano et al., J. Controlled Release 36:125-133, 1995; Chun and Kim, J. Controlled Release 38:39-47, 1996; D'Emanuele and Dinarvand, Int'l J. Pharm. 118:237-242, 1995; Katono et al., J. Controlled Release 16:215-228, 1991; Hoffman, “Thermally Reversible Hydrogels Containing Biologically Active Species,” in Migliaresi et al. (eds.), Polymers in Medicine III, Elsevier Science Publishers B. V., Amsterdam, 1988, pp. 161-167; Hoffman, “Applications of Thermally Reversible Polymers and Hydrogels in Therapeutics and Diagnostics,” in Third International Symposium on Recent Advances in Drug Delivery Systems, Salt Lake City, Utah, Feb. 24-27, 1987, pp. 297-305; Gutowska et al., J. Controlled Release 22:95-104, 1992; Palasis and Gehrke, J. Controlled Release 18:1-12, 1992; Paavola et al., Pharm. Res. 12(12):1997-2002, 1995).”

As is also disclosed in U.S. Pat. No. 6,689,893, “Representative examples of thermogelling polymers, and their gelatin temperature (LCST (° C.)) include homopolymers such as poly(-methyl-N-n-propylacrylamide), 19.8; poly(N-n-propylacrylamide), 21.5; poly(N-methyl-N-isopropylacrylamide), 22.3; poly(N-n-propylmethacrylamide), 28.0; poly(N-isopropylacrylamide), 30.9; poly(N,n-diethylacrylamide), 32.0; poly(N-isopropylmethacrylamide), 44.0; poly(N-cyclopropylacrylamide), 45.5; poly(N-ethylmethyacrylamide), 50.0; poly(N-methyl-N-ethylacrylamide), 56.0; poly(N-cyclopropylmethacrylamide), 59.0; poly(N-ethylacrylamide), 72.0. Moreover thermogelling polymers may be made by preparing copolymers between (among) monomers of the above, or by combining such homopolymers with other water soluble polymers such as acrylmonomers (e.g., acrylic acid and derivatives thereof such as methylacrylic acid, acrylate and derivatives thereof such as butyl methacrylate, acrylamide, and N-n-butyl acrylamide).”

As is also disclosed in U.S. Pat. No. 6,689,893, “Other representative examples of thermogelling polymers include cellulose ether derivatives such as hydroxypropyl cellulose, 41° C.; methyl cellulose, 55° C.; hydroxypropylmethyl cellulose, 66° C.; and ethylhydroxyethyl cellulose, and Pluronics such as F-127, 10-15° C.; L-122, 19° C.; L-92, 26° C.; L-81, 20° C.; and L-61, 24° C.”

As is also disclosed in U.S. Pat. No. 6,689,893, “Preferably, therapeutic compositions of the present invention are fashioned in a manner appropriate to the intended use. Within certain aspects of the present invention, the therapeutic composition should be biocompatible, and release one or more therapeutic agents over a period of several days to months. For example, “quick release” or “burst” therapeutic compositions are provided that release greater than 10%, 20%, or 25% (w/v) of a therapeutic agent (e.g., paclitaxel) over a period of 7 to 10 days. Such “quick release” compositions should, within certain embodiments, be capable of releasing chemotherapeutic levels (where applicable) of a desired agent. Within other embodiments, “low release” therapeutic compositions are provided that release less than 1% (w/v) of a therapeutic agent a period of 7 to 10 days. Further, therapeutic compositions of the present invention should preferably be stable for several months and capable of being produced and maintained under sterile conditions.”

In one preferred embodiment, the anti-mitotic compound is disposed on or in a drug-eluting polymer that is adapted to elute the anti-mitotic compound at a specified rate. These polymers are well known and are often used in conjunction with drug-eluting stents. Reference may be had, e.g., to U.S. Pat. No. 6,702,850 (multi-coated drug-eluting stent), U.S. Pat. No. 6,671,562 (high impedance drug eluting cardiac lead), U.S. Pat. Nos. 6,206,914, 6,004,346 (intralumenl drug eluting prosthesis), U.S. Pat. Nos. 5,997,468, 5,871,535 (intralumenal drug eluting prosthesis), U.S. Pat. Nos. 5,851,231, 5,851,217, 5,725,567, 5,697,967 (drug eluting stent), U.S. Pat. No. 5,599,352 (method of making a drug elting stent), U.S. Pat. No. 5,591,227 (drug eluting stent), U.S. Pat. No. 5,545,208 (intralumenal drug eluting prosthesis), U.S. Pat. No. 5,217,028 (bipolar cardiac lead with drug eluting device), U.S. Pat. No. 4,953,564 (screw-in drug eluting lead), and the like. The entire disclosure of each of these United States patents is hereby incorporated by reference into this specification.

A Process for Delivering the Magnetic Anti-Mitotic Compound

FIG. 1 is a schematic of a preferred process 10 for delivering the magentic anti-mitotic compound described elsewhere in this specification to a specified location. In one embodiment, the magnetic anti-mitotic compound is disposed within a biological organism such as, e.g., a blood vessel 12, and particles 14 of the anti-mitotic compound are delivered to a drug-eluting stent 16.

Referring to FIG. 1, and to the preferred embodiment depicted therein, a bodily fluid, such as blood (not shown for the sake of simplicity of representation) is continuously fed to and through blood vessel 12 in the directions of arrows 20 and 22. In the embodiment depicted, the blood is fed through a generator 26 in order to cause the production of electrical current. In one preferred embodiment, the generator 26 is implanted within an artery 12 or vein 12 of a human being. In another embodiment, not shown, the generator 26 is disposed outside of the artery 12 or vein 12 of the human being.

One may use any of the implanted or implantable generators known to those skilled in the art. Thus, e.g., one may use the power supply disclosed and claimed in U.S. Pat. No. 3,486,506, the entire disclosure of which is hereby incorporated by reference into this specification. This patent claims an electric pulse generator adapted to be implanted within a human body. The generator comprises stator winding means, a permanent magent rotor rotatably mounted adjacent the stator winding means for inducing electrical potentials therein, and means responsive to the movement of the heart for imparting an oscillatory rotary motion to said rotor at approximately the frequency of the heart beat. In one embodiment, the device of U.S. Pat. No. 3,486,506 is a spring-driven cardiac stimulator.

By way of further illustration, the generator 26 may be the heart-actuated generator described and claimed in U.S. Pat. No. 3,554,199, the entire disclosure of which is hereby incorporated by reference in to this specification. Claim 1 of this patent describes: “A device adapted for implantation in the human body for electrically stimulating the heart comprising an envelope housing, an alternating-current generator contained within said housing having a rotor mounted for rotational movement, said rotor having the form of a permanent magnet, a shaft rotatably journaled within said housing, a balance mounted for oscillatory rotational movement about said shaft, the axis of rotation of said rotor being parallel and eccentric to said shaft about which the balance oscillates, a resilient member connected between said housing and the balance, a rotatable member connected with the balance being driven thereby and arranged coaxially with said rotor, a mechanical coupling connecting said rotatable member with said rotor for driving same when said rotatable member is driven by said balance, and electrical contact means connected between said alternating-current generator and the heart muscle for supplying electrical pulses to the heart so as to stimulate the same.”

By way of further illustration, the device disclosed in U.S. Pat. No. 3,563,245 also comprises a miniaturized power supply unit which employs the mechanical energy of heart muscle contractions to produce electrical energy for a pacemaker. This patent claims: “1. A biologically implantable and energized power supply for implanted electric and electronic devices, comprising: a. Fluid pressure sensing means to be disposed inside a heart ventricle for detecting fluid pressure variations therein; b. an energy conversion unit to be disposed outside the heart; c. fluid pressure transfer means connected to said fluid pressure sensing means and to said energy conversion units; said energy conversion unit comprising: d. means for converting said fluid pressure variations into reciprocal motion; e. an electromagnetic generator having a reciprocally rotatable armature; f. means for communicating said reciprocal motion to the reciprocally rotatable armature and thereby convert same therein to corresponding alternating current pulses of electrical energy; g. rectifier means connected to said electromagnetic generator for rectification of said alternating current of electrical energy to corresponding direct current pulses of electrical energy; h. accumulator means connected to said rectifier means for storage therein of the energy in said direct current pulses of electrical energy; and i. connector means connected to said accumulator means for connection thereto of said implanted electric and electronic devices.”

By way of yet further illustration, U.S. Pat. No. 3,456,134 (the entire disclosure of which is hereby incorporated by reference into this specification) discloses a piezoelectric converter for implantable devices utilizing a piezoelectric crystal in the form a a weighted cantilever beam that is adapted to respond to body movement to generate electrical pulses. This patent claims: “1. A converter of body motion to electrical energy for use with electronic implants in the body comrpisng: a closed container of a material not affected by body fluids, a piezoelectric crystal in the form of a cantilevered beam within said container and etending inwardly from a wall of said container with one end anchored in said container wall and the opposite end free to move, a weight mounted on said free end of said crystal cantilvered beam, and means connecting said crystal to the electronic implants in the body.”

As is disclosed in U.S. Pat. No. 3,456,134, when the device of this patent was implanted in the heart of a dog and driven at a mechanical pulse rate of 80 pulses per minute, its produced a maximum output of 4.0 volts at 105 ohms load, or 160 microwatts (see column 2 of the patent).

By way of yet further illustration, the generator 26 may be the piezoelectric converter disclosed in U.S. Pat. No. 3,659,615, the entire disclosure of which is hereby incorporated by reference into this specification. This patent claims: “1. An encapsulated pacesetter implantable in a living system and responsive to movement of an organic muscle to which it is applied to stimulate and pace the natural movement of the muscle, said pacesetter comprising a piezoelectric unit, a transducer, input electrodes electrically connecting said transducer with said generator unit, generator output electrodes for implantation in the muscle tissue, an encapsulating envelope completely enclosing said pacesetter, said envelope formed of a living tissue compatible material consisting of medical grade silicone rubber and a natural wax substantially uniformly and intimately integrated together as a material possessing flexibility sufficient to respond to movement of the muscle tissue in which it is implanted.”

By way of yet further illustration, U.S. Pat. No. 4,453,537 (the entire disclosure of which is hereby incorporated by reference into this specification) discloses a pressure actuated artificial heart powered by a another implanted device attached to a body muscle; the body muscle is stimulated by an electrical signal from a pacemaker. This patent claims: “A device comprising in combination a body implant device and an apparatus for powering said body implant device; said device comprising a reservoir; said reservoir being implantable in the body adjacent to at least one muscle; a fluid disposed within said reservoir; a pressure actuated body implant device; a conduit connecting said reservoir to said body implant device and providing a fluid connection between said reservoir and body implant device; means for periodically stimulating said at least one body muscle from a relaxed state to a contracted state for periodically contracting said at least one body muscle against said reservoir to pressurize said fluid to cause it to flow from said reservoir toward said body implant device; said body implant device including means responsive to said pressurized fluid for powering said body implant device; upon relaxation of said at least one muscle said reservoir returning to its original unpressurized state, thereby creating a vacuum so as to cause the return of said fluid thereto.” As is disclosed in this patent, “The fluid containing reservoir which is implantable in the body and attachable to a body muscle comprises a piston slidably disposed within a cylinder. Preferably, the piston-cylinder reservoir is implanted in the thigh and attached to the rectus femoris muscle . . . . The piston cylinder reservoir is then implanted in the thigh and the insertion end of the muscle is attached to the cylinder and the origin end of the muscle is attached to the piston. The piston-cylinder reservoir is filled with a fluid such as a gas like nitrogen or a liquid such as silicon or oil, and connected to the artificial heart by a biocompatible flexible plastic tubing. Contraction of the rectus femoris muscle forces the piston into the cylinder thereby pressurizing the fluid contained within the cylinder and causing it to flow out of the cylinder and through the flexible plastic tubing toward the artificial heart.”

By way of yet further illustration, U.S. Pat. No. 5,810,015, the entire disclosure of which is hereby incorporated by reference into this specicification, discloses an implantable power supply that is comprised means for converting non-electrical energy to electrical energy. Claim 1 of this patent describes: “1. An implantable power supply apparatus for supplying electrical energy to an electrically powered device, comprising: a power supply unit including:

A. a transcutaneously, invasively rechargeable non-electrical energy storage device (NESD); B. an electrical energy storage device (EESD); and C. an energy converter coupling said NESD and said EESD, said converter including means for converting non-electrical energy stored in said NESD to electrical energy and for transferring said electrical energy to said EESD, thereby storing said electrical energy in said EESD.”

The “prior art” devices for storing non-electrical energy are described at columns 2-4 of U.S. Pat. No. 5,810,015, wherein it is disclosed that: “Any device may be used to store non-electrical energy in accordance with the invention. Many such devices are known which are suitable to act as NESD 22. For example, devices capable of storing mechanical energy, physical phase transition/pressure energy, chemical energy, thermal energy, nuclear energy, and the like, may be used in accordance with the invention. Similarly, any device may be used to store electrical energy in accordance with the invention and to act as EESD 24. Suitable EESDs include, for example, rechargeable batteries and capacitors. Any device capable of converting non-electrical energy to electrical energy may be used to convert energy in accordance with the invention and to act as energy converter 26. When the non-electrical energy used is mechanical energy, for example, energy converter 26 may include a piezoelectric crystal and associated rectifier circuitry as needed. The apparatus of the invention may also include an implanted electrical circuit, such as a driver for a solenoid driven valve, and means for extracting electrical energy from EESD 24 and applying the extracted electrical energy to the electrical circuit.

U.S. Pat. No. 5,810,015 also discloses that: “When the non-electrical energy is mechanical energy, for example, NESD 22 may include a closed fluid system wherein recharging occurs by compression of the fluid. Such a system 10′ is represented in FIGS. 2A and 2B. System 10′ is an implantable medicant infusion pump which includes a biocompatable housing 16 for example, made of titanium, having a piercable septum 18 centrally located in its top surface. A bellows assembly 23 extends from the septum 18 to define a variable volume fluid (or medicant) reservoir 21. A valve/accumulator assembly 30 is coupled between reservoir 21 and an exit cannula 34 to establish a selectively controlled fluid/medicant flow path 34A from the reservoir 21 to a point within the body at the distal tip of cannula 34. In one form of the invention, the valve/accumulator assembly 30 has the form shown in FIG. 3, and includes two solenoid valves 30A, 30B which control the filling and emptying of an accumulator 30C in response signals applied by a controller 32. In response to such signals, the accumulator of assembly 30 drives a succession of substantially uniform pulses of medicant through said catheter 34.”

U.S. Pat. No. 5,810,015 also discloses that: “In the illustrated embodiment, valve/accumulator 30, includes an input port 30′ coupled between reservoir 21 and valve 30A and an output port 30″ coupled between valve 30B and catheter 34. The accumulator includes a diaphragm 31 that is movable between limit surface 33 one side of the diaphragm and limit surface 35 on the other side of the diaphragm. Surface 35 includes open-faced channels therein, defining a nominal accumulator volume that is coupled to valves 30A and 30B. A pressure PB is maintained on the side of diaphragm 31 that is adjacent to surface 35. A pressure of PR is maintained at port 30′, due to the positive pressure exerted on bellows 23 from the fluid in chamber 22A, as described more fully below. A pressure PO is at port 30″, reflecting the relatively low pressure within the patient at the distal end of catheter 34. In operation, the pressure PB is maintained between the PR and PO. Normally, valves 30A and 30B are closed, and diaphragm 31 is biased against surface 33. To generate an output pulse of medicant in catheter 34, valve 30A is opened, and the pressure differential between port 30′ and PB drives fluid into the accumulator 30, displacing the diaphragm 31 to surface 35. The valve 30A is then closed and valve 30B is opened. In response, the pressure differential PB-PO drives an increment of fluid (substantially equal to the previously added fluid) into catheter 34, displacing the diaphragm back to surface 33. Valve 30B then closes, completing the infusion cycle. All valve operations are under the control of controller 32. In other embodiments, other medicant infusion configurations may be used. The controller 32 includes microprocessor-based electronics which may be programmed, for example, by an external handheld unit, using pulse position modulated signals magnetically coupled to telemetry coils within housing 16. Preferably, communication data integrity is maintained by redundant transmissions, data echo and checksums.”

One embodiment of the non-electrical storage device of U.S. Pat. No. 5,810,015 is disclosed in columns 3 et seq. of such patent, wherein it is disclosed that: “In one form of the invention, the bellows assembly 23, together with the inner surface of housing 16, define a variable volume closed fluid chamber 22A which contains a predetermined amount of a gas phase fluid, such as air. The charge of fluid in chamber 22A maintains a positive pressure in the reservoir 21, so that with appropriately timed openings and closings of the valves 30A and 30B, infusate from reservoir 21 is driven through catheter 34. A port 22B couples the chamber 22A to a mechanical-to-electrical energy converter 26, which in turn is coupled to a rechargeable storage battery 24. The battery 24 is coupled to supply power to controller 32 and valves 30A and 30B, and may be used to power other electronic circuitry as desired.”

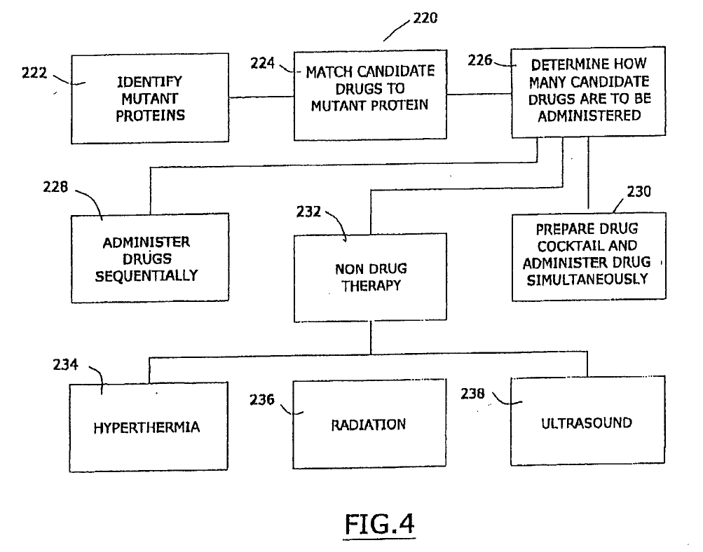

U.S. Pat. No. 5,8100,015 discusses the conversion of mechanical energy to electrical energy at columns 4 et seq., wherein it is disclosed that: “An exemplary mechanical-to-electrical energy converter 26 is shown in FIG. 4. That converter 26 includes a first chamber 26A which is coupled directly via port 22B to chamber 22A, and is coupled via valve 26B, energy extraction chamber 26C, and valve 26D to a second chamber 26E. Energy extraction chamber 26C is preferably a tube having a vaned flow restrictors in its interior, where those flow restrictors are made of piezoelectric devices. A rectifier network 26F is coupled to the piezoelectric devices of chamber 26C and provides an electrical signal via line 26′ to EESD 24. The valves 26B and 26D are operated together in response to control signals from controller 32. When those valves are open, fluid (in gas phase) flows from chamber 22A via chamber 26A and 26C to chamber 26E when the pressure in chamber 22A is greater than the pressure in chamber 26E, and in the opposite direction when the pressure in chamber 22A is less than the pressure in chamber 26E. In both flow directions, the vanes of chamber 26C are deflected by the flowing fluid, which results in generation of an a.c. electrical potential, which in turn is rectified by network 26F to form a d.c. signal used to store charge in EESD 24.”

As is also disclosed in U.S. Pat. No. 5,810,015, “In the operation of this form of the invention, with valves 26B and 26D closed, the chamber 22A is initially charged with fluid, such as air, so that the fluid in chamber 22A exists in gas phase at body temperature over the full range of volume of reservoir 21. Initially, bellows assembly 23 is fully charged with medicant, and thus is fully expanded to maximize the volume of the reservoir 21. The device 10′ is then implanted. After implantation of the device 10′, and valves 26B and 26D are opened, thereby resulting in gas flow through chamber 26C until equilibrium is reached. Then valves 26B and 26D are closed. Thereafter, in response to its internal programming, the controller 32 selectively drives valve/accumulator 30 to complete a flow path between reservoir 21 and cannula, and as described above in conjunction with FIG. 3, driving medicant from reservoir 21, via cannula 34 (and flow path 34A) to a point within the body at a desired rate. In response to that transfer of medicant from reservoir 21, the volume of reservoir 21 decreases, causing an increase in the volume of chamber 22A. As the latter volume increases, a low pressure tends to be established at port 22B. That pressure, with valves 26B and 26D open, in turn draws gas from chamber 26E and through chamber 26C, thereby generating an electrical signal at rectifier 26F. When the reservoir 21 is depleted of medicant, a device such as a syringe may be used to pierce the skin and penetrate the septum 18, and inject a liquid phase medicant or other infusate into reservoir 21, thereby replenishing the medicant in reservoir 21. As liquid is injected into reservoir 21, the bellows assembly 23, expands causing an increase in the volume of reservoir 21 and a decrease in the volume of the phase fluid in chamber 22A, representing storage of mechanical energy. Valves 26B and 26D are then opened, establishing an equilibrating gas flow through chamber 26C, resulting in transfer of charge to EESD 24. In this embodiment, valves 26B and 26D are on opposite sides of chamber 26C. In other embodiments, only one of these valves may be present, and the converter 26 will still function in a similar manner. In yet another embodiment, where chamber 26C has a relatively high flow impedance, there is no need for either of valves 26B and 26D.”

U.S. Pat. No. 5,810,015 also discloses that: “In another form, the bellows assembly 23, together with the inner surface of housing 16, define a variable volume closed fluid chamber 22A which contains a predetermined amount of a fluid, such as freon, which at normal body temperatures exists both in liquid phase and gas phase over the range of volume of chamber 22A. Preferably, the fluid in reservoir 22A is R-11 Freon, which at body temperature 98.6° F. and in a two phase closed system, is characterized by a vapor pressure of approximately 8 psi, where the ratio of liquid-to-gas ratio varies with the volume of chamber 22A. The charge of fluid in chamber 22A maintains a positive pressure in the reservoir 21, so that with appropriately timed openings and closings of the valves 30A and 30B, infusate from reservoir 21 is driven through catheter 34. A port 22B couples the chamber 22A to a mechanical-to-electrical energy converter 26, which in turn is coupled to a rechargeable storage battery 24. The battery 24 is coupled to supply power to controller 32 and valve 30A and 30B. The mechanical-to-electrical energy converter 26 is the same as that described above and as shown in FIG. 4. In this form of the invention, the non-electrical energy is referred to as physical phase transition/pressure energy. In the operation of this form of the invention, the chamber 22A is initially charged with fluid, such as Freon R-11, so that the fluid in chamber 22A exists in both liquid phase and gas phase at body temperature over the full range of volume of reservoir 21. Initially, bellows assembly 23 is fully charged with medicant and thus fully expanded to maximize the volume of reservoir 21. The device is then implanted. Then after implantation of the device 10′, in response to its internal programming, the controller 32 selectively drives valve/accumulator 30 to complete a flow path between reservoir 21 and cannula, and as described above, in conjunction with FIG. 3, to drive medicant from reservoir 21, via cannula 34 (and flow path 34A) to a point within the body at a desired rate. In response to that transfer of medicant from reservoir 21, the volume of reservoir 21 decreases, causing an increase in the volume of chamber 22A. As the latter volume increases, a low pressure tends to be established at port 22B prior to achievement of equilibrium. That pressure, with valves 26B and 26D open, in turn draws gas from chamber 26E and through chamber 26C, thereby generating an electrical signal at rectifier 26F. As the reservoir 21 is depleted of medicant, a device such as a syringe may be used to pierce the skin and penetrate the septum 18, followed by injection of a liquid phase medicant or other infusate into reservoir 21, thereby replenishing the medicant in reservoir 21. As liquid is injected into reservoir 21, the bellows assembly expands causing an increase in the volume of reservoir 21 and a decrease in the volume of the two phase fluid in chamber 22A. That results in an increase in pressure at port 22B representing storage of mechanical energy. Valves 26B and 26D are then opened, establishing an equilibrating gas flow through chamber 26C, resulting in storage of charge in EESD 24. As the bellows assembly 23 is expanded, the re-compression of chamber 22A effects a re-charge of battery 24. The rectifier 26F establishes charging of battery 24 in response to forward and reverse gas flow caused by the expansion and contraction of bellows assembly 23. The present embodiment is particularly useful in configurations similar to that in FIG. 2A, but where the various components are positioned within housing 16 so that the converter 26 normally is higher than the liquid-gas interface in chamber 22A. When implanted, and where the user is upright. With that configuration, and appropriately charged with Freon, the fluid within converter 26 is substantially all in gas phase. In order to prevent liquid phase Freon from passing to chamber 26C when the user is prone, a gravity activated cut-off valve (not shown) may be located in port 22B.”

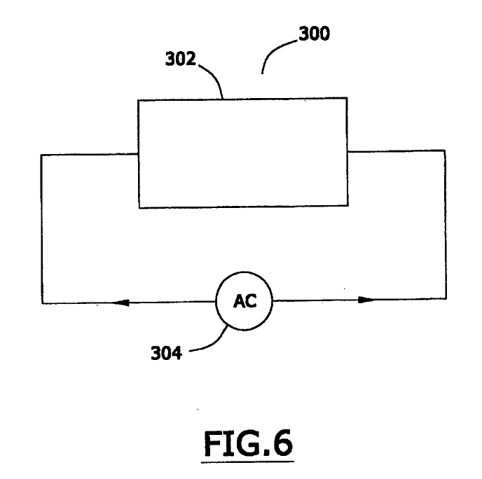

Other implantable devices for converting mechanical energy to electrical energy are discussed at columns 6 et seq. of U.S. Pat. No. 5,810,015. Thus, e.g., it is disclosed that: “In another embodiment in which mechanical energy is stored in NESD 22, shown in FIG. 6, NESD 22 includes a compressible spring 41B. Spring 41B is connected to a compressor assembly 43 which may be accessed transcutaneously. Any means may be used to compress spring 41B. As shown in FIG. 6, compressor 43 includes a screw which may be turned by application of a laparoscopic screwdriver 45.

As is also disclosed in U.S. Pat. No. 5,810,015, “When the non-electrical energy stored in NESD 22 is chemical energy, NESD 22 includes a fluid activatable chemical system. Recharging may occur by injection of one or more chemical solutions into NESD 22. Any chemical solutions may be used to store chemical energy in NESD 22 in accordance with this embodiment of the invention. For example, a solution of electrolytes may be used to store chemical energy in NESD 22.”

U.S. Pat. No. 5,810,015 also discloses that: “When the non-electrical energy stored in NESD 22 is thermal energy, NESD 22 includes a thermal differential energy generator capable of generating electrical energy when a fluid having a temperature greater than normal mammalian body temperature is injected into the generator. By way of example, a Peltier effect device may be used, where application of a temperature differential causes generation of an electrical potential. Alternatively, a bimetallic assembly may be used where temperature-induced mechanical motion may be applied to a piezoelectric crystal which in turn generates an electrical potential.”

U.S. Pat. No. 5,810,015 also discloses that: “In another embodiment, the invention provides a method of supplying energy to an electrical device within a mammalian body which comprises implanting into the mammal an apparatus including a power supply having: a transcutaneously rechargeable NESD; an EESD; and an energy converter coupling said rechargeable means and the storage device, where the converter converts non-electrical energy stored in the NESD to electrical energy and transfers the electrical energy to the EESD, thereby storing the electrical energy in the EESD; and transcutaneously applying non-electrical energy to the NESD. Any of the devices described above may be used in the method of the invention.”

Referring again to FIG. 1, and to the preferred embodiment depicted therein, the blood preferably flows in the direction of arrow 20, past generator 26, and through stent assembly. The electrical energy from generator 26 is passed via line 28 to regulator 30.

In one referred emodiment, the generator 26 produces alternating current that is converted into direct current by regulator 30. One may use, e.g., any of the implantable rectifiers known to those skiled in the art as regulator 30.

These prior art implantable rectifiers are well known and are described, e.g., in U.S. Pat. No. 5,999,849, the entire disclosure of which is hereby incorporated by reference into this specification. As is disclosed in this patent, medical devices that are configured to perform a desired medical function are often implanted in the living tissue of a patient so that a desired function may be carried out as needed for the benefit of the patient. “Numerous examples of implantable medical devices are known in the art, ranging from implantable pacemakers, cochlear stimulators, muscle stimulators, glucose sensors, and the like. Some implantable medical devices are configured to perform the sensing function, i.e., to sense a particular parameter, e.g., the amount of a specified substance in the blood or tissue of the patient, and to generate an electrical signal indicative of the quantity or concentration level of the substance sensed. Such electrical signal is then coupled to a suitable controller, which may or may not be implantable, and the controller responds to the sensed information in a way to enable the medical device to perform its intended function, e.g., to display and/or record the measurement of the sensed substance. An example of an implantable medical device that performs the sensing function is shown, e.g., in U.S. Pat. No. 4,671,288.”

As is also disclosed in U.S. Pat. No. 5,999,849, “As medical devices have become more useful and numerous in recent years, there is a continual need to provide very low power sensors that may be connected to, or incorporated within, such devices so that the desired function of the device can be carried out without the expenditure of large amounts of power (which power, for an implanted device, is usually limited.) It is known in the art to inductively couple a high frequency ac signal into an implanted medical device to provide operating power for the circuits of the device. Once received within the implanted device, a rectifier circuit, typically a simple full-wave or half-wave rectifier circuit realized with semiconductor diodes, is used to provide the rectifying function. Unfortunately, when this is done, a significant signal loss occurs across the semiconductor diodes, i.e., about 0.7 volts for silicon, which signal loss represents lost power, and for low level input signals of only a volt or two represents a significant decrease in the efficiency of the rectifier. For the extremely low power implantable devices and sensors that have been developed in recent years, low operating voltages, e.g., 2-3 volts, are preferable in order to keep overall power consumption low. Unfortunately, with such low operating voltages are used, a diode voltage drop of 0.7 volts represents a significant percentage of the overall voltage, thus resulting in a highly inefficient voltage rectification or conversion process. An inefficient voltage conversion, in turn, translates directly to increased input power, which increased input power defeats the overall design goal of the low power device. What is needed, therefore, is a low power rectifier circuit that efficiently converts a low amplitude alternating input signal to a low output operating voltage.” The device described and claimed in U.S. Pat. No. 5,999,849 is: “1. A low power switched rectifier circuit comprising: first and second voltage rails (120, 122); a storage capacitor (C1) connected between the first and second voltage rails; first and second input lines (LINE 1, LINE 2); a first switch (M1) connecting the first input line to the first voltage rail; a second switch (M2) connecting the second input line to the first voltage rail; a third switch (M3) connecting the first input line to the second voltage rail; a fourth switch (M4) connecting the second input line to the second voltage rail; a detector circuit for each of said first, second, third, and fourth switches, respectively, powered by voltage on the storage capacitor, that automatically controls its respective switch to close and open as a function of the voltage signal appearing on the first input line relative to the second input line such that, in concert, the first and fourth switches close and the second and third switches open in response to a positive signal on the first input line relative to the second input line, and such that second and third switches close and the first and fourth switches open in response to a negative signal on the first input line relative to the second input line, whereby the first input line is automatically connected to the first voltage rail and the second input line is automatically connected to the second voltage rail whenever a positive signal appears on the first input line relative to the second input line, and whereby the first input line is automatically connected to the second voltage rail and the second input line is automatically connected to the first voltage rail whenever a negative signal appears on the first input line relative to the second input line; and startup means for supplying the storage capacitor with an initial voltage sufficient to power each of the detector circuits; said low power switched rectifier circuit wherein all of said first, second, third, and fourth switches and respective detector circuits are all part of a single integrated circuit.”

Thus, by way of further illustration, reference to U.S. Pat. No. 6,456,883, the entire disclosure of which is hereby incorporated by reference into this specification, one may use the implantable rectifier disclosed in such patent. This patent claims, e.g., “36. A method for providing an electrical power feed selection for an implantable medical device comprising: transmitting radio frequency signals to an antenna of the implantable medical device; rectifying the radio frequency signals by a rectifier circuit; storing energy contained in the transmitted radio frequency signals in a supplemental power source that comprises an energy storage device; comparing voltage levels of an electrical main power source and the supplemental power source and outputting a signal from a comparator indicating which power source is greater; receiving a signal from the comparator and selecting the supplemental power source as a power feed when the main power source is depleted; and maintaining the voltage level from the supplemental power source at a predetermined level when the supplemental power source has been selected as the power feed . . . .”

Referring again to FIG. 1, and in one preferred embodiment thereof, the regulator 30 is operatively connected to controller 32 by means of a link 34, and the regulator 30 is comprised of an andjustable power supply whose output may be regulated in response to signals fed to such regulator 30 by controller 32.

One may use any of the implantable power supplies known to those in the art as regulator 32. Thus, e.g., one may use the biologically implantable and energized power supply disclosed in U.S. Pat. No. 3,563,245, the entire disclosure of which is hereby incorporated by reference into this specification.

Thus, by way of further illustration, one may use the power supply disclosed in U.S. Pat. No. 3,757,795, the entire disclosure of which is hereby incorporated by reference into this specification. Claim 6 of this patent describes: “6. Implantable electrical medical apparatus including circuit means for developing electrical signals for stimulating selected portions of a body, comprising: electrically redundant power supply means having a pair of supply junctions; means connecting said circuit means to said supply junctions; voltage doubling means having first and second output terminals adapted to be connected to a body for electrical stimulation thereof; said voltage doubling means including a capacitor having a pair of plates; means connecting one of said plates to one of said supply junctions; means connecting the other of said plates to said first output terminal; means connecting said second output terminal to the other supply junction; electrical switch means connecting said one plate to said other supply junction; further electrical switch means connecting said second output terminal to said one supply junction; and all said switch means being connected to said circuit means and including means for selectably reversing the polarity of electrical energy to said capacitor.”

By way of yet further illustration, one may use the power supply disclosed in U.S. Pat. No. 4,143,661, the entire disclosure of which is hereby incorporated by reference into this specification. As is dislosed in the abstract of this patent, “A power supply system to operate an implanted electric-powered device such as a blood pump. A secondary coil having a biocompatible covering is implanted to subcutaneously encircle either the abdomen or the thigh at a location close to the exterior skin. The secondary coil is electrically interconnected with an implanted storage battery and the blood pump. A primary coil of overlapping width is worn by the patient at a location radially outward of the secondary coil. An external battery plus an inverter circuit in a pack is attached to a belt having a detachable buckle connector which is conventionally worn about the waist. Efficient magnetic coupling is achieved through the use of two air-core windings of relatively large diameter.”

In the specification of U.S. Pat. No. 4,143,661, some of the preferred embodiments of the invention of such patent are discussed. It is disclosed that: “This invention relates to electric power supplies and more particularly to a power supply for a device which is implanted within a living body and a method for operation thereof. The relatively high amount of power required by circulatory support devices, such as a partial or total artificial heart, has rendered most implantable, self-sufficient energy sources inapplicable, such as those used for a pacemaker. Only high-power, radioisotope heat sources have held any promise of sustained outputs of several watts; however, the utilization of such an energy source has been complicated by its inherent need for a miniature, high efficiency heat engine, as well as by serious radiation-related problems. All other practical approaches to powering an artificial heart or circulatory assist system of some type necessarily depend on a more or less continuous flow of energy from outside the body. Results of early efforts at infection-free maintenance of long-term percutaneous connections were discouraging and thus highlighted the desirability, at least for the long term, of powering such an implanted device though intact skin.”

As is also disclosed in U.S. Pat. No. 4,143,661, “One of the earliest approaches to the transmission of energy across intact skin involves the generation of a radio frequency field extending over a substantial area of the body, such that significant power could be extracted from coils located in the vicinity of the implanted power-consuming device itself. Placement of substantial amounts of ferrite materials within such coils to permit the capture of a greater proportion of the incident field was also investigated, as reported in the article by J. C. Schuder et al. in the 1964 Transactions ACEMB. However, difficulty has been experienced in reconciling the conflicting requirement of magnetic circuit geometry with a surgically feasible, variable tissue structure. In another proposed alternative design, a secondary coil is implanted in such a manner that the center of the coil remains accessible through a surgically constructed tunnel of skin; however, such devices have not yielded satisfactory performance. Predominant failure modes included necrosis of the skin tunnel tissue caused by mechanical pressure and excess heat generation—see the 1975 report of I.I.T. Research Institute, by Brueschke et al., N.I.H. Report No. NO1-HT-9-2125-3, page 25.”

U.S. Pat. No. 4,143,661 also discloses that: “As a result of the present invention, it has been found that a satisfactory system can be achieved by the employment of a secondary coil which is implanted just below the skin of the abdomen or the thigh so that it encircles the body member along most of its length and lies at a location close to the skin. The system includes an implanted storage battery plus the necessary interconnections between the secondary coil, the battery and the electric-powered device, which will likely be a circulatory assist device of some type. A primary coil, in the form of an encircling belt which is greater in width than the secondary implanted coil, fits around the body member in the region just radially outward thereof. A portable external A.C. power source, usually a rechargeable battery plus an appropriate inverter, is in electrical connection with the primary coil. These coils function efficiently as an air-core transformer and sufficient power is transcutaneously supplied via the secondary coil to both operate the device and charge the implanted storage battery.”

By way of yet further illustration, one may use the power supply described in U.S. Pat. No. 4,665,896, the entire disclosure of which is hereby incorporated by reference into this specification. This patent claims: “1. In an implanted blood pump system wherein power for driving the pump is provided by a transcutaneous transformer having an external primary winding means and an implanted secondary winding means and shunt regulator means for controlling the driving voltage applied to the pump, a method for regulating the driving voltage applied to the primary winding means, comprising, sensing the power factor in the primary winding means, comparing the sensed power factor to a predetermined power factor level selected to correspond with a desired pump driving voltage, and adjusting the voltage level in the primary winding means to substantially equalize the sensed power factor and the predetermined power factor level.”

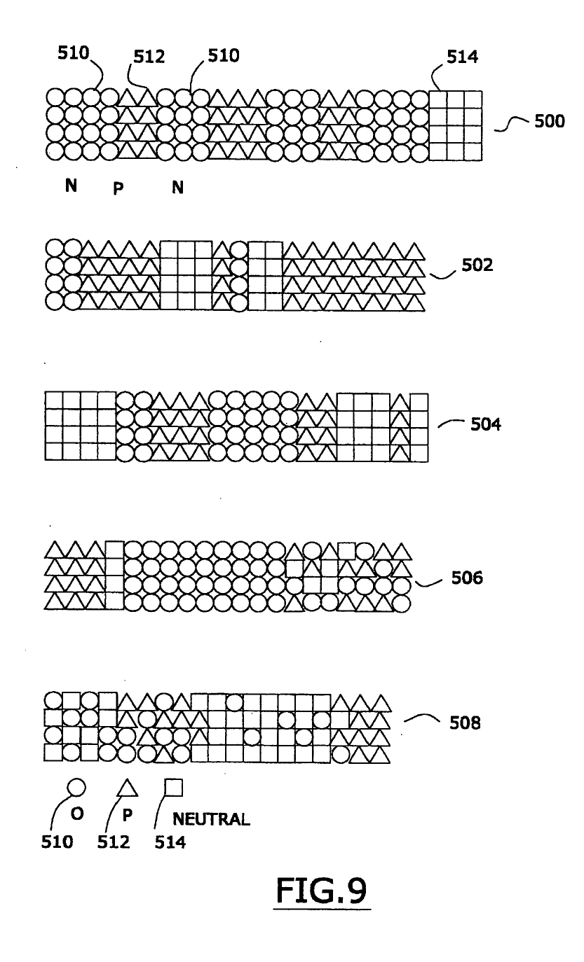

By way of yet further illustration, one may use the surgically implanted power supply described in U.S. Pat. No. 5,702,430, the entire disclosure of which is hereby incorporated by reference into this specification. This patent claims: “1. A surgically implantable power supply comprising battery means for providing a source of power, charging means for charging the battery means, enclosure means isolating the battery means from the human body, gas holding means within the enclosure means for holding gas generated by the battery means during charging, seal means in the enclosure means arranged to rapture when the internal gas pressure exceeds a certain value and inflatable gas container means outside the enclosure means to receive gas from within the enclosure means when the seal means has been ruptured.” As is discussed in the specification of this patent, a rectifier device may be used with the claimed assembly. Thus, e.g., it is disclosed that: “Power for the internal battery charging circuit is obtained via a subcutaneous secondary coil 230. This coil is connected to a capacitor/rectifier circuit 231 that is tuned to the carrier frequency being transmitted transcutaneously to the secondary coil 230. The rectifier may incorporate redundant diodes and a fault detection circuit as shown, which operates similar to the power transistor bridge 222 and logic circuit 223 of FIG. 9(a), except that the power transistors are replaced by diodes. This tuned capacitor/rectifier circuit may also incorporate a filter arrangement 211 to support serial communication interface (SCI) reception via the secondary coil 230. A level detection comparator 232 is provided to convert the analog signal produced by the filter 211 into a digital signal compatible with an SCI receiver 460. A power transistor 233 or other modulation device may also be incorporated to support SCI transmission via the secondary coil 230. A redundant transistor bridge such as the bridge 222 used for PWM current limiting may be used in place of the transistor 233 for improved fault tolerance. This SCI interface provides for changing programmable settings used by the control algorithm and monitoring of analog inputs to the microcontroller such as ECG1, ECG2, MCH1, CUR1, CUR2, TEMP, V1, and V2.”

By way of yet further illustration, one may use the power supply disclosed in U.S. Pat. No. 5,949,632, the entire disclosure of which is hereby incorporated by reference into this specification. This patent claims: “Power for the internal battery charging circuit is obtained via a subcutaneous secondary coil 230. This coil is connected to a capacitor/rectifier circuit 231 that is tuned to the carrier frequency being transmitted transcutaneously to the secondary coil 230. The rectifier may incorporate redundant diodes and a fault detection circuit as shown, which operates similar to the power transistor bridge 222 and logic circuit 223 of FIG. 9(a), except that the power transistors are replaced by diodes. This tuned capacitor/rectifier circuit may also incorporate a filter arrangement 211 to support serial communication interface (SCI) reception via the secondary coil 230. A level detection comparator 232 is provided to convert the analog signal produced by the filter 211 into a digital signal compatible with an SCI receiver 460. A power transistor 233 or other modulation device may also be incorporated to support SCI transmission via the secondary coil 230. A redundant transistor bridge such as the bridge 222 used for PWM current limiting may be used in place of the transistor 233 for improved fault tolerance. This SCI interface provides for changing programmable settings used by the control algorithm and monitoring of analog inputs to the microcontroller such as ECG1, ECG2, MCH1, CUR1, CUR2, TEMP, V1, and V2.”

By way of yet further illustration, one may use the power supply described in U.S. Pat. No. 5,954,058, the entire disclosure of which is hereby incorporated by reference into this specification. This patent claims: “A rechargeable electrically powered implantable infusion pump and power unit therefor, for intracorporeally dispensing a liquid in a body of a living being, with said infusion pump and power until therefor being capable of subcutaneous implantation in said body of said living being, said infusion pump and power unit comprising: A. a rigid or semi-rigid outer pump housing; B. a flexible liquid storage chamber inside said outer-pump housing for containing a liquid to be dispensed intracorporeally in the body of said being by said infusion pump, said liquid storage chamber having a variable volume and a transcutaneously accessible self-sealing inlet and outlet port in communication with said outer-pump housing, such that said liquid can alternatively be introduced into said chamber through said port to refill said chamber, and be pumped out of said chamber through said port upon actuation of electrically powered infusion pump means for intracorporeally dispensing said liquid in the body of said being; C. electrically powered infusion pump means for causing said liquid to be pumped out of said liquid storage chamber through said port thereof and dispensed within said body of said living being upon actuation of said infusion pump means; D. a charging fluid storage chamber at least in part surrounding said liquid storage chamber and containing a two phase charging fluid, wherein the liquid phase to gas phase ratio of said charging fluid is representative of a store of potential energy in the form of physical phase transition/pressure energy which is transferrable into kinetic energy upon the physical phase transition of said charging fluid due to the vaporization of said charging fluid form its liquid phase to its vapor phase; E. rechargeable electrical energy source means contained within said outer-pump housing, for rechargeably receiving and storing electrical energy and for supplying said stored electrical energy to power said infusion pump means; and F. energy converter means in communication with both said charging fluid storage chamber and said rechargeable electrical energy source means, and contained within said outer-pump housing, for converting the released physical phase transition/pressure potential energy of said charging fluid to said electrical energy and for supplying said electrical energy to said rechargeable electrical energy source means.”

By way of yet further illustration, one may use the adjustable power supply described in U.S. Pat. No. 6,141,583, the entire disclosure of which is hereby incorporated by reference into this specification. As is discussed in the abstract of this patent, there is disclosed “A method or apparatus for conserving power in an implantable medical device (IMD) of the type having at least one IC powered by a battery wherein, in each such IC, a voltage dependent oscillator for providing oscillator output signals at an oscillation frequency dependent upon applied supply voltage to the IC is incorporated into the IC. The voltage dependent oscillator oscillates at a frequency that is characteristic of the switching speed of all logic circuitry on the IC die that can be attained with the applied supply voltage. The applied supply voltage is regulated so that the oscillation frequency is maintained at no less than a target or desired oscillation frequency or within a desired oscillation frequency range. The power supply voltage that is applied to the IC is based directly on the performance of all logic circuitry of the IC. In order to provide the comparison function, the oscillator output signals are counted, and the oscillator output signal count accumulated over a predetermined number of system clock signals is compared to a target count that is correlated to the desired oscillation frequency. The counts are compared, and the supply voltage is adjusted upward or downward or is maintained the same dependent upon whether the oscillator output signal count falls below or rises above or is equal to the target count, respectively. The supply voltage adjustment is preferably achieved employing a digitally controlled power supply by calculating a digital voltage from the comparison of the oscillator output signal count to the target count, and storing the digital voltage in a register of the power supply.”

Referring again to FIG. 1, and in the preferred embodiment depicted therein, the generator 26, in one embodiment, produces alternating current This alternating current is fed via line 28 to regulator 30, which preferably converts the alternating current to direct current and either feeds it in a first direction via line 36 to metallic stent 16, or feeds it in another direction via line 38 to metallic stent 16. As will be apparent to those skilled in the art, the regulator 26 thus has the capability of producing a magnetic field of a first polarity (when the direct current is fed in a first direction 36) or a second polarity (swhen the direct current is fed in a second direction 38), as dictated by the well-known Lenz's law.

In one embodiment, the regulator 26 is capable not only of changing the direction of the electrical current, but also its amount. It preferably is comprised of a variable resistance circuit that can modulate its output.

In the preferred embodiment depicted, the regulator 26 is comprised of a transceiver (not shown) whose antenna 40 is in telemetric contact with a controller 32. The controller 32 is preferably in telemetric contact with biosensors 42, 44, 46, and/or 48; and, depending upon the information received from one or more of such sensors, can direct the regulator 30 to increase the production of electrical current in one direction, or another, to decrease the production of electrical current in one direction, or another, or to cease the production of electrical current in one direction or another.

Biosensors 42, 44, 46, and/or 48 may be one or more of the implantable biosensors known to those skilled in the art.

In one embodiment, one of such sensors 42, 44, 46, and/or 48 can determine the extent to which two recognition molecules have bound to each other. Thus, e.g., one may use the process and apparatus described in U.S. Pat. No. 5,376,556, in which an analyte-mediated ligand binding event is monitored; the entire disclosure of this United States patent is hereby incorporated by reference into this specification. Claim 1 of this patent describes “A method for determining the presence or amount of an analyte, if any, in a test sample by monitoring an analyte-mediated ligand binding event in a test mixture the method comprising: forming a test mixture comprising the test sample and a particulate capture reagent, said particulate capture reagent comprising a specific binding member attached to a particulate having a surface capable of inducing surface-enhanced Raman light scattering and also having attached thereto a Raman-active label wherein said specific binding member attached to the particulate is specific for the analyte, an analyte-analog or an ancillary binding member; providing a chromatographic material having a proximal end and a distal end, wherein the distal end of said chromatographic material comprises a capture reagent immobilized in a capture situs and capable of binding to the analyte; applying the test mixture onto the proximal end of said chromatographic material; allowing the test mixture to travel from the proximal end toward the distal end by capillary action; illuminating the capture situs with a radiation sufficient to cause a detectable Raman spectrum; and monitoring differences in spectral characteristics of the detected surface-enhanced Raman scattering spectra, the differences being dependent upon the amount of analyte present in the test mixture.”

By way of further illustration, one may use the “triggered optical sensor” described and claimed in U.S. Pat. No. 6,297,059, the entire disclosure of which is hereby incorporated by reference into this specification. This patent claims (in claim 1) thereof “. An optical biosensor for detection of a multivalent target biomolecule comprising: a substrate having a fluid membrane thereon; recognition molecules situated at a surface of said fluid membrane, said recognition molecule capable of binding with said multivalent target biomolecule and said recognition molecule linked to a single fluorescence molecule and as being movable upon said surface of said fluid membrane; and, a means for measuring a change in fluorescent properties in response to binding between multiple recognition molecules and said multivalent target biomolecule.” In column 1 of this patent, other biological sensors are discussed, it being stated that: “Biological sensors are based on the immobilization of a recognition molecule at the surface of a transducer (a device that transforms the binding event between the target molecule and the recognition molecule into a measurable signal). In one prior approach, the transducer has been sensitive to any binding, specific or non-specific, that occurred at the transducer surface. Thus, for surface plasmon resonance or any other transduction that depended on a change in the index of refraction, such sensors have been sensitive to both specific and non-specific binding. Another prior approach has relied on a sandwich assay where, for example, the binding of an antigen by an antibody has been followed by the secondary binding of a fluorescently tagged antibody that is also in the solution along with the protein to be sensed. In this approach, any binding of the fluorescently tagged antibody will give rise to a change in the signal and, therefore, sandwich assay approaches have also been sensitive to specific as well as non-specific binding events. Thus, selectivity of many prior sensors has been a problem. Another previous approach where signal transduction and amplification have been directly coupled to the recognition event is the gated ion channel sensor as described by Cornell et al., ‘A Biosensor That Uses Ion-Channel Switches’, Nature, vol. 387, Jun. 5, 1997. In that approach an electrical signal was generated for measurement. Besides electrical signals, optical biosensors have been described in U.S. Pat. No. 5,194,393 by Hugl et al. and U.S. Pat. No. 5,711,915 by Siegmund et al. In the later patent, fluorescent dyes were used in the detection of molecules.”

By way of yet further illustration, one may use the sensor element disclosed in U.S. Pat. No. 6,589,731, the entire dislcosure of which is hereby incorporated by reference into this specification. This patent, at column 1 thereof, also discusses biosensors, stating that: “Biosensors are sensors that detect chemical species with high selectivity on the basis of molecular recognition rather than the physical properties of analytes. See, e.g., Advances in Biosensors, A. P. F. Turner, Ed. JAI Press, London, (1991). Many types of biosensing devices have been developed in recent years, including enzyme electrodes, optical immunosensors, ligand-receptor amperometers, and evanescent-wave probes. The detection mechanism in such sensors can involve changes in properties such as conductivity, absorbance, luminescence, fluorescence and the like. Various sensors have relied upon a binding event directly between a target agent and a signaling agent to essentially turn off a property such as fluorescence and the like. The difficulties with present sensors often include the size of the signal event which can make actual detection of the signal difficult or affect the selectivity or make the sensor subject to false positive readings. Amplification of fluorescence quenching has been reported in conjugated polymers. For example, Swager, Accounts Chem. Res., 1998, v. 31, pp. 201-207, describes an amplified quenching in a conjugated polymer compared to a small molecule repeat unit by methylviologen of 65; Zheng et al., J. Appl. Polymer Sci., 1998, v. 70, pp. 599-603, describe a Stern-Volmer quenching constant of about 1000 for poly(2-methoxy,5-(2′-ethylhexloxy)-p-phenylene-vinylene (MEH-PPV) by fullerenes; and, Russell et al., J. Am. Chem. Soc., 1982, v. 103, pp. 3219-3220, describe a Stern-Volmer quenching constant for a small molecule (stilbene) in micelles of about 2000 by methylviologen. Despite these successes, continued improvements in amplification of fluorescence quenching have been sought. Surprisingly, a KSV of greater than 105 has now been achieved.”

Similarly, and by way of further illustration, one may use the light-based sensors discussed at column 1 of U.S. Pat. No. 6,594,011, the entire disclosure of which is hereby incorporated by reference into this specification. As is disclosed in such columm 1, “It is well known that the presence or the properties of substances on a material's surface can be determined by light-based sensors. Polarization-based techniques are particularly sensitive; ellipsometry, for example, is a widely used technique for surface analysis and has successfully been employed for detecting attachment of proteins and smaller molecules to a surface. In U.S. Pat. No. 4,508,832 to Carter, et al. (1985), an ellipsometer is employed to measure antibody-antigen attachment in an immunoassay on a test surface. Recently, imaging ellipsometry has been demonstrated, using a light source to illuminate an entire surface and employing a two-dimensional array for detection, thus measuring the surface properties for each point of the entire surface in parallel (G. Jin, R. Janson and H. Arwin, “Imaging Ellipsometry Revisited: Developments for Visualization of Thin Transparent Layers on Silicon Substrates,” Review of Scientific Instruments, 67(8), 2930-2936, 1996). Imaging methods are advantageous in contrast to methods performing multiple single-point measurements using a scanning method, because the status of each point of the surface is acquired simultaneously, whereas the scanning process takes a considerable amount of time (for example, some minutes), and creates a time lag between individual point measurements. For performing measurements where dynamic changes of the surface properties occur in different locations, a time lag between measurements makes it difficult or impossible to acquire the status of the entire surface at any given time. Reported applications of imaging ellipsometry were performed on a silicon surface, with the light employed for the measurement passing through +the surrounding medium, either air or a liquid contained in a cuvette. For applications where the optical properties of the surrounding medium can change during the measurement process, passing light through the medium is disadvantageous because it introduces a disturbance of the measurement.”

U.S. Pat. No. 6,594,011 goes on to disclose (at columns 1-2) that: “By using an optically transparent substrate, this problem can be overcome using the principle of total internal reflection (TIR), where both the illuminating light and the reflected light pass through the substrate. In TIR, the light interacting with the substance on the surface is confined to a very thin region above the surface, the so-called evanescent field. This provides a very high contrast readout, because influences of the surrounding medium are considerably reduced. In U.S. Pat. No. 5,483,346 to Butzer, (1996) the use of polarization for detecting and analyzing substances on a transparent material's surface using TIR is described. In the system described by Butzer, however, the light undergoes multiple internal reflections before being analyzed, making it difficult or impossible to perform an imaging technique, because it cannot distinguish which of the multiple reflections caused the local polarization change detected in the respective parts of the emerging light beam. U.S. Pat. No. 5,633,724 to King, et al. (1997) describes the readout of a biochemical array using the evanescent field. This patent focuses on fluorescent assays, using the evanescent field to excite fluorescent markers attached to the substances to be detected and analyzed. The attachment of fluorescent markers or other molecular tags to the substances to be detected on the surface requires an additional step in performing the measurement, which is not required in the current invention. The patent further describes use of a resonant cavity to provide on an evanescent field for exciting analytes.”

By way of yet further illustration, one may use one or more of the biological sensors disclosed in U.S. Pat. No. 6,546,267 (biological sensor), U.S. Pat. No. 5,972,638 (biosensor), U.S. Pat. Nos. 5,854,863, 6,411,834 (biological sensor), U.S. Pat. No. 4,513,280 (device for detecting toxicants), U.S. Pat. Nos. 6,666,905, 5,205,292, 4,926,875, 4,947,854 (epicardial multifunctional probe), U.S. Pat. Nos. 6,523,392, 6,169,494 (biotelemetry locator), U.S. Pat. No. 5,284,146 (removable implanted device), U.S. Pat. Nos. 6,624,940, 6,571,125, 5,971,282, 5,766,934 (chemical and biological sensosrs having electroactive polymer thin films attached to microfabricated device and possessing immobilized indicator molecules), U.S. Pat. No. 6,607,480 (evaluation system for obtaining diagnostic information from the signals and data of medical sensor systems), U.S. Pat. Nos. 6,493,591, 6,445,861, 6,280,586, 5,327,225 (surface plasmon resonance sensor), and the like. The entire disclosure of each of these United States patents is hereby incorporated by reference into this specification.

By way of further illustration, one may use the implantable extractable probe described in U.S. Pat. No. 5,205,292, the entire disclosure of which is hereby incorporated by reference into this specification. This probe comprises a biological sensor attached to the body of the probe such as, e.g., a doppler transducer for measuring blood flow.

In one embodiment, the nanowire sensor described in published U.S. patent application U.S. 20020117659 is used; the entire disclosure of this United States patent application is hereby incorporated by reference into this specification. As is disclosed in this published patent aplication, “The invention provides a nanowire or nanowires preferably forming part of a system constructed and arranged to determine an analyte in a sample to which the nanowire(s) is exposed. ‘Determine’, in this context, means to determine the quantity and/or presence of the analyte in the sample. Presence of the analyte can be determined by determining a change in a characteristic in the nanowire, typically an electrical characteristic or an optical characteristic. E.g. an analyte causes a detectable change in electrical conductivity of the nanowire or optical properties. In one embodiment, the nanowire includes, inherently, the ability to determine the analyte. The nanowire may be functionalized, i.e. comprising surface functional moieties, to which the analytes binds and induces a measurable property change to the nanowire. The binding events can be specific or non-specific. The functional moieties may include simple groups, selected from the groups including, but not limited to, —OH, —CHO, —COOH, —SO3H, —CN, —NH2, SH, —COSH, COOR, halide; biomolecular entities including, but not limited to, amino acids, proteins, sugars, DNA, antibodies, antigens, and enzymes; grafted polymer chains with chain length less than the diameter of the nanowire core, selected from a group of polymers including, but not limited to, polyamide, polyester, polyimide, polyacrylic; a thin coating covering the surface of the nanowire core, including, but not limited to, the following groups of materials: metals, semiconductors, and insulators, which may be a metallic element, an oxide, an sulfide, a nitride, a selenide, a polymer and a polymer gel. In another embodiment, the invention provides a nanowire and a reaction entity with which the analyte interacts, positioned in relation to the nanowire such that the analyte can be determined by determining a change in a characteristic of the nanowire.”

A drug delivery device that is comprised of a biological sensor is disclosed in published United States patent application U.S. 2002/011601, the entire disclosure of which is hereby incorporated by reference into this specification. As is disclosed in the “Abstract” of this published patent application, “An Implantable Medical Device (IMD) for controllably releasing a biologically-active agent such as a drug to a body is disclosed. The IMD includes a catheter having one or more ports, each of which is individually controlled by a respective pair of conductive members located in proximity to the port. According to the invention, a voltage potential difference generated across a respective pair of conductive members is used to control drug delivery via the respective port. In one embodiment of the current invention, each port includes a cap member formed of a conductive material. This cap member is electrically coupled to one of the conductive members associated with the port to form an anode. The second one of the conductive members is located in proximity to the port and serves as a cathode. When the cap member is exposed to a conductive fluid such as blood, a potential difference generated between the conductors causes current to flow from the anode to the catheter, dissolving the cap so that a biologically-active agent is released to the body. In another embodiment of the invention, each port is in proximity to a reservoir or other expandable member containing a cross-linked polymer gel of the type that expands when placed within an electrical field. Creation of an electric field between respective conductive members across the cross-linked polymer gel causes the gel to expand. In one embodiment, this expansion causes the expandable member to assume a state that blocks the exit of the drug from the respective port. Alternatively, the expansion may be utilized to assert a force on a bolus of the drug so that it is delivered via the respective port. Drug delivery is controlled by a control circuit that selectively activates one or more of the predetermined ports.”

At column 1 of published U.S. patent application U.S. 2002/0111601, reference is made to other implantable drug delivery systems. It is disclosed that (in paragraph 0004) that “While implantable drug delivery systems are known, such systems are generally not capable of accurately controlling the dosage of drugs delivered to the patient. This is particularly essential when dealing with drugs that can be toxic in higher concentrations. One manner of controlling drug delivery involves using electro-release techniques for controlling the delivery of a biologically-active agent or drug. The delivery process can be controlled by selectively activating the electro-release system, or by adjusting the rate of release. Several systems of this nature are described in U.S. Pat. Nos. 5,876,741 and 5,651,979 which describe a system for delivering active substances into an environment using polymer gel networks. Another drug delivery system is described in U.S. Pat. No. 5,797,898 to Santini, Jr. which discusses the use of switches provided on a microchip to control the delivery of drugs. Yet another delivery device is discussed in U.S. Pat. No. 5,368,704 which describes the use of an array of valves formed on a monolithic substrate that can be selectively activated to control the flow rate of a substance through the substrate.” The disclosures of each of U.S. Pat. Nos. 5,368,704, 5,797,898, and 5,876,741 are hereby incorporated by reference into this specification.

In one embodiment, and referring again to FIG. 1, sensor 36 is an electromagnetic flow meter that, as is known to those skilled in the art, is an instrument which is used to qualitiatively measure flow velocity. Reference may be had to a text by J. A. Tuszynski et al., “Biomedical Applications of Introductory Physics”(John Wiley & Sons, Inc., New York, N.Y., 2001), at page 260.

In another embodiment, and referring again to FIG. 1, the sensor 36 is adapted to detect the degree of oxygenation of blood in accordance with the procedure described in U.S. Pat. No. 6,690,958, the entire disclosure of which is hereby incorporated by reference into this specification. This patent claims, in its claim 1”. A diagnostic apparatus comprising: a near infrared spectrophotometer comprising one or more optical sources and one or more optical detectors capable of interrogating one or more optical source volumes; an ultrasound transducer capable of interrogating an ultrasound source volume, the optical sources, the optical detectors, and the ultrasound transducer being configured in a line so that the one or more optical source volumes and the ultrasound source volume are coplanar and the one or more optical source volumes intersect the ultrasound source volume; and a moveable fixture coupled to the one or more optical sources, the one or more optical detectors, and the ultrasound transducer, and capable of adjustment to vary optical source-detector distances of respective one or more optical sources and one or more optical detectors.” As is disclosed in the abstract of this patent, “A diagnostic apparatus includes a near infrared spectrophotometer (NIRS) and an ultrasound transducer that operate in combination to improve diagnostic measurements. The diagnostic apparatus includes a near infrared spectrophotometer that measures an analyte, for example tissue oxygenation, in an optical sample volume and an ultrasound imager to accurately position the optical sample volume in biological tissue or vessels. In one example, the diagnostic apparatus includes an optical source, a linear array of ultrasound transducers, and an optical photodetector arranged in the same plane so that the ultrasound sample volume interrogated by the ultrasound transducers intersects the optical sample volume formed by the optical source and detector.”

FIG. 2 is a schematic diagram of an electromagnetic flow meter applied to an artery; this Figure is adapted from page 261 of the aforementioned Tuszynski et al. text. Referring such FIG. 2, it will be seen that blood (not shown) flows through artery 100 in the direction of arrow 102. A first signal electrode 102 at a first voltage potential is electrically connected to a second signal electrode (not shown) at a second voltage potential. A magentic field in the direction of arrows is created by magnet 108. As blood flows in the direction of arrow 102 and between the first signal electrode 102 and the second signal electrode (not shown), a current is induced by such flow, and such current is measured by a galvanometer (not shown) that is part of the sensor 36 (see FIG. 1).

In addition to the device depicted in FIG. 2, or instead of such device, one may use one or more of the implantable flow meters known to the prior art. Thus, e.g., one may use one or more of the implantable flow meters disclosed in U.S. Pat. No. 4,915,113 (method and apparatus for monitoring the patency of vascular grants), U.S. Pat. No. 6,458,086 (implantable blood flow monitoring system), U.S. Pat. No. 6,668,197 (treatement using implantable devices), U.S. Pat. No. 6,824,480 (monitoring treatment using implantable telemetric sensors), and the like. The entire disclosure of each of these United States patents is hereby incorporated by reference into this specification.

By way of further illustration, claim 1 of U.S. Pat. No. 4,915,113 describes: an “Implantable flow meter apparatus for monitoring vascular graft patency comprising: (a) at least one ring member for surrounding a blood vessel graft intermediate its ends, said at least one ring member supporting transducer means thereon to define an axis extending internal of said blood vessel graft when said at least one ring member is installed on said blood vessel graft; (b) implantable electrical means positionable subcutaneously at a predetermined access site displaced from said one at least one ring member; (c) conductor means coupling said transducer means to said electrical means; and (d) barrier patch means having an area much greater than the cross-sectional area of said conductor means, said conductor means passing generally through the center of said patch means for inhibiting passage of infection producing organisms from said access site along said conductor means.”

Referring again to FIG. 1, and in the preferred embodiment depicted, a growth of plaque 41 is shown. As will be apparent, and for the sake of simplicity of representation, the plaque 41 is shown on only one portion of the stent 30.

As is known to those in the art, and as is illustrated at page 135 of the Tuszynski et al. text (see problem 11.9), when a segment of an artery is narrowed down by arterisclerotic plaque to one fifth of its cross-sectional area, the velocity increases five times; but the blood pressure increases about about 1 percent.

Thus, e.g., if one were to use the flow-meter depicted in FIG. 2, and assuming a magnetic field of about 10 Gauss, a blood flow rate of about 20 centimeters per second, a diamter of the artery 100 of about 1 centimeter, the voltage difference between the first electrode 104 and the second electrode (not show) will be about 1.5 millivolts; and the current flow will be proportional to the resistance in the circuit formed by the two electrodes. With e.g., a 5 ohm resistance, the current would be about 0.3 milliamperes.

Referring again to FIG. 1, when such current of about 0.3 milliamperes is detected by the sensor 42, such information is preferably transmitted by such sensor 42 to the controller 32. The controller 32 then can determine, based upon this infomration and other information, to what extent, if any, it wishes to change the activity of regulator 30.

Referring again to FIG. 1, and in the embodiment depicted, the stent 16 also is preferably comprised of sensors 44, 46, and 48. One or more of these sensors may be adapted to detect the amount of anti-mitotic agent in the bloodstream.

Referring again to FIG. 1, and to the preferred embodiment depicted therein, particles of magnetic anti-mitotic agent 14 are fed into the artery 11 by means of source 50. These magnetic particles are directed by an externally applied magnetic field 52 towards the stent 16. As will be apparent, the stent 16 will also have a magnetic moment, depending upon the direction in which current is fed from regulator 30 to the stent 16. When the magnetic momement of the stent is opposite to that of the magnetic anti-mitotic particles 14, the anti-mitotic particles are attracted to the stent 16; when the magnetic moment of the stent 16 is the same as that of the anti-mitotic particles 14, the anti-mitotic particles are directed to the stent. Thus, the controller 32 can control the extent to which, if any, the stent 16 attracts and/or repels the magnetic anti-mitotic particles in its vicinity.

Similarly, when externally applied magnetic field 52 has a magnetic moment that is opposite to that of the magentic particles, these particles can be driven towards the stent; and they can be pulled from the stent when the externally applied magnetic field has an opposite orientation.

Thus, there are two separate factors that can be varied to either draw the magentic anti-mitotic particles towards the stent, or to repel such anti-mitotic particles from the stent: the strength and orientation of the magnetic field of the stent (which is controllable via regulator 30), and the strength and orientation of the externally applied magnetic field 52.

One may use any of prior art means for externally applying magnetic field 52. Thus, and referring to published U.S. patent application 2004/0030379, the entire disclosure of which is hereby incorporated by reference into this specification, “An external electromagnetic source or field may be applied to the patient having an implanted coated medical device using any method known to skilled artisan. In the method of the present invention, the electromagnetic field is oscillated. Examples of devices which can be used for applying an electromagnetic field include a magnetic resonance imaging (“MRI”) apparatus. Generally, the magnetic field strength suitable is within the range of about 0.50 to about 5 Tesla (Webber per square meter). The duration of the application may be determined based on various factors including the strength of the magnetic field, the magnetic substance contained in the magnetic particles, the size of the particles, the material and thickness of the coating, the location of the particles within the coating, and desired releasing rate of the biologically active material.”

Published U.S. patent application 2004/0030379 also disclose that “In an MRI system, an electromagnetic field is uniformly applied to an object under inspection. At the same time, a gradient magnetic field, superposing the electromagnetic field, is applied to the same. With the application of these electromagnetic fields, the object is applied with a selective excitation pulse of an electromagnetic wave with a resonance frequency which corresponds to the electromagnetic field of a specific atomic nucleus. As a result, a magnetic resonance (MR) is selectively excited. A signal generated is detected as an MR signal. See U.S. Pat. No. 4,115,730 to Mansfield, U.S. Pat. No. 4,297,637 to Crooks et al., and U.S. Pat. No. 4,845,430 to Nakagayashi. For the present invention, among the functions of the MRI apparatus, the function to create an electromagnetic field is useful for the present invention. The implanted medical device of the present can be located as usually done for MRI imaging, and then an electromagnetic field is created by the MRI apparatus to facilitate release of the biologically active material. The duration of the procedure depends on many factors, including the desired releasing rate and the location of the inserted medical device. One skilled in the art can determine the proper cycle of the electromagnetic field, proper intensity of the electromagnetic field, and time to be applied in each specific case based on experiments using an animal as a model.”

Published U.S. patent application 2004/0030379 also disclose that “In addition, one skilled in the art can determine the excitation source frequency of the elecromagnetic energy source. For example, the electromagnetic field can have an excitation source frequency in the range of about 1 Hertz to about 300 kiloHertz. Also, the shape of the frequency can be of different types. For example, the frequency can be in the form of a square pulse, ramp, sawtooth, sine, triangle, or complex. Also, each form can have a varying duty cycle.”

Referring again to FIG. 1, and in the preferred embodiment depicted therein, In the embodiment depicted, a layer of drug eluting polymer 49 is present in the stent assembly; and this polymer may be used to either attract anti-mitotic agent into it, and/or to elute anti-mitotic agent out of it.

In one preferred embodiment, direct current electrical energy is delivered via lines 36/38 to to stent assembly 16. In this emboidment, it is preferred that stent assembly 16 be comprised of conductive material, and that the stent also be comprised of wire-like struts (see, e.g., FIG. 1 of published United States patent application 1004/0030379).

As will be apparent, as the direct current flows through the conductive material, it creates a static magnetic field in accordance with the well-known Lenz's law. In one embodiment, with the blood flow that is typical through the blood vessels of human beings, magnetic fields on the order of about 1. Gauss can readily be created.

Referring again to FIG. 1, the stent assembly 16 is preferably comprised of a metallic stent body 16 and, disposed thereon, drug eluting polymer 49. The hydrodynamic forces caused by the flow of blood through the stent assembly 16 causes elution of particles 14 of anti-mitotic agent.

It is preferred that regulator 30 be comprised of either a half wave or a full wave rectifier so that the current flowing from regulator 30 be direct current, i.e., that such current flow in only one direction. As will be apparent with either “half-wave d.c.” and/or “full-wave d.c.” being fed to the stent 16, a magnetic field will be induced in such stent that will have a constant polarity but constantly varying intensity. Such a magnetic field with either consistently attract and/or repel the magnetic anti-mitotic particles 14, depending upon the magnetic polarity of such particles. In one preferred embodiment, the magnetized stent 16 consistently attracts the magnetic particles 14.

As will be apparent, the regulator is capable of varying the intensity and/or polarity of its output, preferably in response to a signal from the controller 32. The controller 32 is preferably equipped with an antenna 50 which is in telemetric contact with both the regulator 30 and the sensors 42, 44, 46, and 48.

The sensors 42, 44, 46, and 48 may be any of implantable biosensors known to those skilled in the art.

By way of illustration, and referring to U.S. Pat. No. 4,915,113 (the entire disclosure of which is hereby incorporated by reference into this specification), the sensor(s) may be a implantable Dopper flow meter apparatus for monitoring blood flow through a vascular graft. This patent claims: “1. Implantable flow meter apparatus for monitoring vascular graft patency comprising: (a) at least one ring member for surrounding a blood vessel graft intermediate its ends, said at least one ring member supporting transducer means thereon to define an axis extending internal of said blood vessel graft when said at least one ring member is installed on said blood vessel graft; (b) implantable electrical means positionable subcutaneously at a predetermined access site displaced from said one at least one ring member; (c) conductor means coupling said transducer means to said electrical means; and (d) barrier patch means having an area much greater than the cross-sectional area of said conductor means, said conductor means passing generally through the center of said patch means for inhibiting passage of infection producing organisms from said access site along said conductor means.”

The sensor(s) may comprise a means for sensing the strength of a magnetic field. As is disclosed in claim 4 of U.S. Pat. No. 5,562,714 (the entire disclosure of which is hereby incorporated by reference into this specification), the sensing means “ . . . comprises a sensing antenna having an electrical connection through diodes to a power supply so that the Q of said transmitting antenna is regulated by draw down of energy by said sense antenna through said diode connection to said power supply.

In one embodiment, depicted in FIG. 3, the energy fed via line 24 is direct-current electrical energy. In this embodiment,

A Process for Predicting Mutation Type and Mutation Frequency

In one embodiment of applicants' invention, there is provided a process for predicting both the type and frequency of mutations in certain protein drug targets.

As is known to those skilled in the art, many mutations are “silent,” i.e., they do not result in amino acid changes in the protein being expressed. Put another way, a silent mutation is a mutation that does not result in a detectable phenotypic effect. A silent mutation may be due to a transition or a transversion that leads to synonym codon. Additionally, mutations can change a codonto code for an amino acid closely related in terms of shape, hydrophobicity or other properties to that coded for by the original codon. Reference may be had, e.g., to U.S. Pat. Nos. 5,240,846 5,639,650; 5,840,493 (mitochondrial DNA mutations); U.S. Pat. No. 5,976,798 (methods for detecting mitochondrial mutations); U.S. Pat. No. 6,010,908 (gene therapy by small fragment homologous replacement); U.S. Pat. No. 6,329,138 (method for the detection of antibiotic resistance); U.S. Pat. No. 6,344,356 (methods for recombining nucleic acids); U.S. Pat. No. 6,544,745 (diagnostic assay for diabetes); U.S. Pat. No. 6,699,479; and the like. The entire disclosure of each of these United States patents is hereby incorporated by reference into this specification.

An additional preferred embodiement is an algorithm using artificial intelligence or computer programs that improve their performance based on information gathered from previous cycles to predict which DNA bases are most likely to be mutated and result in important amino acid changes. This information can be derived empirically from data gathered by the sequencing of tubulin mutants from clinical samples of tumors.

As is also known to those skilled in the art, the active site of a protein is assembled from many amino acids that interact with the substrate of the enzymatic reaction or ligand binding reactions. In one embodiment of applicants' invention, one can anticipate which amino acid changes will result in a change in drug binding. In one aspect of this embodiment, one anticipates which amino acid changes result in changes in drug binding in paclitaxeal and, thereafter, designs drugs to bind to the modified binding sites. In this aspect, by utilizing such drugs in advance of the mutation event, or concurrently therewith, the incidence of selecting for resistant forms of cancer is minimized.

Applicants' process 200 is schematically illustrated in FIG. 3. In step 202 of the process, the structure of the target protein is obtained. The target protein may, e.g., be a beta-tubulin that is implicated in, e.g., certain drug resistance.

One may obtain the structure of the target protein by conventional or unconventional means. One, thus, may conduct conventional x-ray crystallography analysis of the protein in question. Alternatively, or additionally, one may obtain and/or confirm the structure of the protein in question by homology modeling, as is discussed elsewhere in this specification.

Thereafter, in step 204 of the process, the binding efficiency of a candidate drug to the target protein is predicted by conventional means. One may use the means disclosed in U.S. Pat. No. 5,854,992 (system and method for structure-based drug design that includes accurate prediction of binding free energy); U.S. Pat. No. 5,933,819 (prediction of relative biding moits of biologically active peptides and peptide mimetics); U.S. Pat. No. 6,226,603 (method for the prediction of binding targets and the design of ligands); U.S. Pat. No. 6,772,073 (method for prediction of binding targets and the design of ligands); and the like. The entire disclosure of each of these United States patents is hereby incorporated by reference into this specification.

By way of illustration, and referring to U.S. Pat. No. 5,854,992, such patent claims: “1. A method for building molecules for binding at a receptor site, comprising the steps of: (a) evaluating a receptor site for a molecular make up of at least a portion of the receptor site to which a molecule being grown will bind and generating at least a coordinate of at least a portion of the receptor site to which the molecule being grown will bind, and outputting, at least with respect to the molecular make up of the receptor site, the coordinate of the portion of the receptor site to which the molecule being grown will bind; (b) estimating free energy of the molecule being grown using knowledge-based potential data to estimate free energy and outputting the estimated free energy; and (c) building a molecule for binding to the receptor site using the outputs from steps (a) and (b), with the building step including building the molecule by selecting molecular fragments at orientations that will result in free energy estimates for the molecule that may be higher than a lowest free energy estimate possible for the molecule.”

Thereafter, in step 206 of the process, the key amino acids that are essential for the interaction of the target protein and the candidate drug are identified. This step also may be conducted by conventional means, such as evaluation of the results of the energy minimization analyses preferably conducted in step 204.

In step 208 of the process, a slight variation in the homology model is made in order to determine how the modified model will function. Thus, e.g., one may modify the target protein used in step 202, and then the process is repeated to determine the binding efficiency of the candidate drug (in step 204) for the modified target protein. The process is then repeated again, and again, until a multiplicity of sets of data are obtained with a multiplicity of different target proteins for the same drug.

This multiplicity of data will indicate which target protein the drug is most efficiently bound to the candidate drug, and which target protein is least efficiently bound to the target drug. The least efficiently bound target proteins are those proteins that, through natural selection of cells, might cause drug resistance to the candidate drug. Thus, in step 210, the data from repeated runs of process 200 is evaluated to determine which of the target proteins are least likely to bind to the candidate drug.

In step 212, the candidate drug is modified, and the modified drug is then tested again in the cyle of steps 202/204/206/208 to determine its binding efficiency with each of the target proteins initially evaluated as well as other modified target proteins.

This process may lead to other modified candidate drugs. The goal is to test for, and determine, the existence of a modified drug that has a high binding efficiency for all of the targeted protein structures.

As will be apparent, the process depicted in FIG. 3 may be used to determine drugs that may minimize drug resistance to anti-mitotic agents; and these “modified drugs” may be used either by themselves and/or in combination with the original cancer drug, depending upon the relative binding efficiencies with regard to particular target proteins and the extent to which the use of such drugs results in synergy. As will also be apparent, the process depicted in FIG. 3 may be used to determine drugs that may minimize other drug resistance caused by natural selection, such as antibiotic drug resistance. The process may also be used in cases of herbicide resistance, pesticide resistance, resistance to antiviral drugs, etc.

FIG. 4 is a flow diagram of one particular process 220 involving the design of anti-mitotic drugs and, in one embodiment thereof, combinations of antimitotic drugs. Referring to FIG. 4, and in step 222 thereof, the mutant proteins that are resistant to certain anti-mitotic agents are identified. These mutant proteins can be identified by conventional means such as, e.g., those means described hereinbelow, which relate to the identification of mutant tubulin isotypes.

Some of these mutant tubulin isotypes are discussed in published U.S. patent application 2004/0121351, the entire disclosure of which is hereby incorporated by reference into this specification. This published United States patent application discloses that: “The conservation of structure and regulatory functions among the P-tubulin genes in three vertebrate species (chicken, mouse and human) allowed the identification of and categorization into six major classes of beta-tubulin polypeptide isotypes on the basis of their variable carboxyterminal ends . . . . As tubulin molecules are involved in many processes and form part of many structures in the eucaryotic cell, they are possible targets for pharmaceutically active compounds. As tubulin is more particularly the main structural component of the microtubules it may act as point of attack for anticancer drugs such as vinblastin, colchicin, estramustin and taxol which interfere with microtubule function. The mode of action is such that cytostatic agents such as the ones mentioned above, bind to the carboxyterminal end the β-tubulin which upon such binding undergoes a conformational change. For example, Kavallaris et al. [Kavallaris et al. 1997, J. Clin. Invest. 100: 1282-1293] reported a change in the expression of of specific β-tubulin isotypes (class I, II, III, and IVa) in taxol resistant epithelial ovarian tumor. It was concluded that these tubulins are involved in the formation of the taxol resistence. Also a high expression of class III P-tubulins was found in some forms of lung cancer suggesting that this isotype may be used as a diagnostic marker.”

The function of certain tubulins in paclitaxel resistance was also discussed in U.S. Pat. No. 6,362,321, the entire disclosure of which is hereby incorporated by reference into this specification. As is disclosed in this patent, “Taxol is a natural product derived from the bark of Taxus brevafolio (Pacific yew). Taxol inhibits microtubule depolymerization during mitosis and results in subsequent cell death. Taxol displays a broad spectrum of tumorcidal activity including against breast, ovary and lung cancer (McGuire et al., 1996, N. Engld. J. Med. 334:1-6; and Johnson et al., 1996, J. Clin. Ocol. 14:2054-2060). While taxol is often effective in treatment of these malignancies, it is usually not curative because of eventual development of taxol resistance. Cellular resistance to taxol may include mechanisms such as enhanced expression of P-glycoprotein and alterations in tubulin structure through gene mutations in the β chain or changes in the ratio of tubulin isomers within the polymerized microtubule (Wahl et al., 1996, Nature Medicine 2:72-79; Horwitz et al., 1993, Natl. Cancer Inst. 15:55-61; Haber et al., 1995, J. Biol. Chem. 270:31269-31275; and Giannakakou et al., 1997, J. Biol. Chem. 272:17118-17125) . . . .”

The increased presence of certain tubulin isotypes associated with certain types of cancers was noted in an article by Tien Yeh et al., “The B_{II} Isotype of Tubulin is Present in the Cell Nuclei of a Variety of Cancers,” Cell Motility and the Cytoskeleton 57:96-106 (2004). The Yeh et al. article discloses that both alpha-tubulin and beta-tubulin consist of a series of isotypes differing in amino acid sequence, each one encoded by a different gene; and it refers to a 1998 article by Richard F. Luduena entitled “The multiple forms of tubulin: different gene products and covalent modifications,” Int. Rev. Cytol 178:207-275. The Yeh et al. article also disclosed that the B_{II} isotype of tubulin is present in the nuclei of many tumors, stating that “Three quarters (75%) of the tumors we examined contained nuclear the B_{II} (Table I).” The authors of the Yeh et al. article suggest that (at page 104) “ . . . it would be interesting to expore the possibility of using nuclear B_{II} as a chemotherapeutic target.”

The aforementioned articles disclose several conventional means for identifying mutant proteins that are a cause, at least in part, of anti-mitotic drug resistance. Comparable means may be used to identify mutant proteins that are the cause of antibioitic drug resistance, vaccine resistance, herbicide reistance, pesticide resistance, antiviral drug resitance, and the like. In general, one may study specimens of drug resistant orgnanisms to determine the existence of prorteins that are preferentially expressed in the drug resistant organisms as compared with a comparable non-drug resistant organisms. Additionally, or alteratively, one may determine the existence of proteins that are preferentially expressed in the diseased organisms in order to determine whether such proteins are essential for the progress of the disease. Means for making such determinations are well documented in the patent literature. Reference may be had, e.g., to U.S. Pat. No. 5,853,995 (large scale genotyping of diseases); U.S. Pat. No. 6,162,604 (methods for determining genetic predisposition to automimmune disease by genotypying apoptotic genes); U.S. Pat. No. 6,291,175 (methods for treating a neurological disease by determining BCHE genotype); U.S. Pat. No. 6,303,307 (large scale genotyping of disease); U.S. Pat. Nos. 6,355,859; 6,432,643 (method of determining Alzheimer's disease risk using apolipoprotein E4 genotype analysis); U.S. Pat. No. 6,573,049 (genotyping of the paraoxonase 1 gene for prognosing, diagnosing, and treating a disease); and the like. The entire disclosure of each of these United States patents is hereby incorporated by reference into this specification.

Referring again to FIG. 4, and in step 224 thereof, certain candidate drugs are then identified that will bind to the mutant proteins. This can be done with the process depicted in FIG. 3.

It is often the case that more than one mutant protein is present in cases of drug resistance. As is known, cancer often has a heterogeneous genotype in which different isotopes preferentially contain different drug-resistant proteins. In such a case, it is often desirable to determine not only which candidate drugs will bind to the particular mutant protein (see step 222), but also what combination of drugs will effectively bind to all the mutant proteins present in the heterogeneous genotype. Furthermore, one should also determine the concentration(s) and/or ratios of such drugs to maximize the possibility of a synergistic therapeutic effect.

After the identity and concentration of the drugs to be used has been determined, one can can either administer these drugs simultaneously (in step 228) and/or administer these drugs sequentially (in step 230). Additionally, or alternatively, in step 232 one may administer non-drug therapy either the same time as the administration of the drug(s), and/or at one or more different times.

One may use one or more of non-drug anti-mitotic therapies that are known to those skilled in the art. Thus, e.g., in step 234 one may use hyperthermia. With the use of the magnetic anti-mitotic drugs discussed elsewhere in this specification, one may direct these drugs to the site of a tumor with the aid of an external electromagnetic field and thereafter, with the use of one or more other electromagnetic fields, cause such drug(s) to heat up to its Curie temperature and preferentially damage and/or destroy cancer cells. In one aspect of this embodiment, the Curie temperature of the magnetic anti-mitotic compound is less than about 41 degrees Celsius.

One may use radiation therapy in step 236. Thus, e.g., the magnetic anti-mitotic drug of this invention may contain a radioactive moiety, such as radioactive iron, or radioactive cobalt.

One may use ultrasound therapy is step 238. This step is described in more detail in the next section of this specification.

Treatment of In Vivo Tumors with High Frequency Energy

FIG. 5 is a flow diagram of a preferred process 260 for treating a biological organism with mechanical vibrational energy (such as ultrasound) as set forth in step 238 of FIG. 4.

In the process of applicants' invenition, in addition to the ultrasound energy, one may use other forms of mechanical energy, some of which are disclosed in published U.S. patent application 2004/0030379.

Referring to published U.S. patent application 2004/0030379, the entire disclosure of which is hereby incorporated by reference into this specification, “The mechanical vibrational energy source includes various sources which cause vibration such as ultrasound energy. Examples of suitable ultrasound energy are disclosed in U.S. Pat. No. 6,001,069 to Tachibana et al. and U.S. Pat. No. 5,725,494 to Brisken, PCT publications WO00/16704, WO00/18468, WO00/00095, WO00/07508 and WO99/33391, which are all incorporated herein by reference. Strength and duration of the mechanical vibrational energy of the application may be determined based on various factors including the biologically active material contained in the coating, the thickness of the coating, structure of the coating and desired releasing rate of the biologically active material.”

As is also disclosed in published U.S. patent application 2004/0030379, “Various methods and devices may be used in connection with the present invention. For example, U.S. Pat. No. 5,895,356 discloses a probe for transurethrally applying focused ultrasound energy to produce hyperthermal and thermotherapeutic effect in diseased tissue. U.S. Pat. No. 5,873,828 discloses a device having an ultrasonic vibrator with either a microwave or radio frequency probe. U.S. Pat. No. 6,056,735 discloses an ultrasonic treating device having a probe connected to a ultrasonic transducer and a holding means to clamp a tissue. Any of those methods and devices can be adapted for use in the method of the present invention.”

As is also disclosed in published U.S. patent application 2004/0030379, “Ultrasound energy application can be conducted percutaneously through small skin incisions. An ultrasonic vibrator or probe can be inserted into a subject's body through a body lumen, such as blood vessels, bronchus, urethral tract, digestive tract, and vagina. However, an ultrasound probe can be appropriately modified, as known in the art, for subcutaneous application. The probe can be positioned closely to an outer surface of the patient body proximal to the inserted medical device.”

As is also disclosed in published U.S. patent application 2004/0030379, “The duration of the procedure depends on many factors, including the desired releasing rate and the location of the inserted medical device. The procedure may be performed in a surgical suite where the patient can be monitored by imaging equipment. Also, a plurality of probes can be used simultaneously. One skilled in the art can determine the proper cycle of the ultrasound, proper intensity of the ultrasound, and time to be applied in each specific case based on experiments using an animal as a model.”

As is also disclosed in published U.S. patent application 2004/0030379, “In addition, one skilled in the art can determine the excitation source frequency of the mechanical vibrational energy source. For example, the mechanical vibrational energy source can have an excitation source frequency in the range of about 1 Hertz to about 300 kiloHertz. Also, the shape of the frequency can be of different types. For example, the frequency can be in the form of a square pulse, ramp, sawtooth, sine, triangle, or complex. Also, each form can have a varying duty cycle.”:

Referring to FIG. 5, and in step 261 thereof, the cells of a biological organism to be treated are first preferably synchronized. so that they are experiencing substantially synchronous growth; in one aspect of this embodiment, such cells are synchronized in metaphase.

As is known to those skilled in the art, synchronous growth is growth in which all (or a substantial porition) of the cells are at the same stage of cell division at a given time; this is also often referred to as “synchronized growth.” Reference may be had, e.g., to page 471 of J. Stensch's “Dictionary of Biochemistry and Molecular Biology,” Second Edition (John Wiley & Sons, New York 1989). Reference may also be had, e.g., to U.S. Pat. No. 5,18,887, the entire disclosure of which is hereby incorporated by reference into this specification.

Referring to such U.S. Pat. No. 5,158,887, in claim 15 thereof there is described “0.15. The process as set forth in claim 1, wherein said modified cell elongation and synchronization of growth in the number of said cells and their effective mass is accomplished by: carrying out at least one additional subculture and incubation step between steps (c) and (d) of claim 1 wherein in each instance a batch subculture is prepared which contains a quantity of said slowly metabolizable carbon source in a growth medium and bacterial cells obtained from the immediately preceding batch subculture at a density level no greater than about one half of the density of the bacterial cells present in the immediately preceding batch subculture, and the batch subculture thus prepared incubated for a time to cause the cells therein to multiply only about one to one and one half generations.” The “claim 1.” of such patent referred to in such claim 15 describes “1. 1. A process for producing bacterial cells useful in selective production of spores and a metabolic end product selected from the group consisting of solvents, enzymes, antibiotics and useful toxic proteins, and comprising the steps of: providing an initial stock culture containing a carbon source in a growth medium, and at least about 1×106 cells per milliliter of bacteria of the genus Clostridium, said bacterial cells, when treated to inhibit division, being genetically capable of metabolizing a carbon source to produce spores or a metabolic end product selected from the group consisting of said solvents, enzymes, antibiotics and proteins; providing a quantity of a divalent cation source; inducing elongation of said bacterial cells under conditions to produce modified cells of a critical length of at least about 3× while synchronizing the growth in the number of said cells and their effective mass by (a) preparing from the initial stock culture another batch subculture which contains a quantity of a slowly metabolizable carbon source other than glucose in a growth medium by adding to the other batch subculture bacterial cells obtained from the initial stock culture and present at a density level no greater than about one half of the density of the bacterial cells present in the initial stock culture; (b) incubating said other batch subculture within a time to cause the cells therein to multiply for only about one to one and one half generations in said batch subculture while maintaining the growth medium at a temperature within a range of about −20° C. to +10 ° C. of the species specific optimum growth temperature, said growth medium being devoid of an amount of cellular metabolites that would be sufficient to substantially interfere with synchronous growth of said cells, (c) preparing from an immediately preceding batch subculture a final batch subculture which contains a quantity of a slowly metabolizable carbon source other than glucose in a growth medium by adding to said final batch subculture bacterial cells obtained from the immediately preceding batch subculture and present at a density level no greater than about one half of the density of the bacterial cells present in said immediately preceding batch subculture; (d) incubating said final batch subculture for a time to cause the cells therein to multiply while maintaining the growth medium at a temperature within the range of step (b), said growth medium being devoid of an amount of cellular metabolites that would be sufficient to substantially interfere with synchronous growth of said cells, and (e) carrying out at least incubation step (d) in the presence of at least about 0.01M of said divalent cation and which is sufficient to cause cellular incorporation of an amount of said divalent cation into said elongated cells during step (d) to stabilize the cells against death, lysis and aggregation and cause modified cell division in a manner such that, as each cell divides into two cells, the resulting divided cells remain elongated to at least said 3× length, said slowly metabolizable carbon source being selected in each instance to cause the bacteria to grow in the selected growth medium at a rate of about 10%-90% less than the maximum growth rate Km for the bacteria in an optimum growth medium; and thereafter selectively subjecting the cells resulting from step (d) to treatment conditions which thereafter inhibit cell division and cause the cells to primarily produce either spores or at least one of said metabolic end products.”

As is well known to those skilled in the art, other means of synchronization of growth of the cells of a biological organism may be used. Reference may be had, e.g., to U.S. Pat. No. 4,315,503 (modification of the growth, repair, and maintenance behavior of living tissues and cells by a specific and selective change in electrical environment), U.S. Pat. No. 4,533,635 (process for stimulating the growth of epidermal cells), U.S. Pat. No. 4,931,053 (method for enhancing vascular and other growth), U.S. Pat. No. 5,158,887 (process for massive conversion of clostridia in synchronized cells), U.S. Pat. No. 6,050,990 (methods and devices for inhibiting hair growth), U.S. Pat. No. 6,143,560 (method of synchronizing epithelial cells into G₀ phase), U.S. Pat. No. 6,149,495 (human fibroblast diffusible factors), U.S. Pat. No. 6,369,294 (methods comprising apoptosis inhibitors for the generation of transgenic pigs), U.S. Pat. No. 6,448,040 (inhibitor of cellular proliferation), U.S. Pat. No. 6,767,734 (method and apparatus for producing age-synchronized cells), and the like. The entire disclosure of each of these United States patents is hereby incorporated by reference into this specification.

In one especially preferred embodiment, and referring again to FIG. 5, in step 261 the cells of biological organisms are synchronized by means of cell cycle arresting drugs. These drugs are well known to those skilled in the art. Reference may be had, e.g., to European patent publication EP 0 870 506 for “Compositons comprising a cryptophicin compound in combination with a synchronizing or activating agent for treating cancer.” As is disclosed in this patent publication, As used herein, the term “synchronizing agent” refers to an agent that can partially synchronize tumor cells with respect to cell cycle progression. Thus the term shall refer to cell cycle phase specific agents such as Gemcitabine, which is now commercially available and other agents such as multitargeted antifolate (MTA, LY231514), the sulfonylurea LY295501, cisplatin, carboplatin, cyclophosphamide, topoisomerase inhibitor, CPT-11, etoposide, VP-16, 5-fluorouracil, doxorubicin, methotrexate, hydroxyurea and 3′-azido-3′-deoxythymidine (AZT). Methods for preparing Gemcitabine are known to the skilled artisan and are described in U.S. Pat. No. 4,808,614, herein incorporated by reference in its entirety. See also, European Patent number EP122707 (Sep. 16, 1987) . . . . As used herein the term “activating agent” refers to an agent that can activate non-cycling cells so that they enter the cell cycle where they will be sensitive to the cytotoxic activity of Compounds I-V and agents which effect growth factor downstream kinase cascade to activate the cell cycle. Examples of activating agents are growth factors, interleukins, and agents which modulate the function of cell cycle regulation which control cell cycle checkpoints and progression through the cell cycle. For example, but not limited to cdc25 phosphatase or p21. (sdil, wafl, cipl). Such growth factors and interleukins are known and readily available to the skilled artisan.”

In one preferred embodiment, the synchronizing agent used is preferably an agent that can partially synchronize tumor cells with respect to cell cycle progression and preferably is a cell cycle phase specific agents such as Gemcitabine, which is now commercially available. Gemcitabine, and its synthesis, are well known to those skilled in the art. Reference may be had, e.g., to U.S. Pat. No. 6,001,994, the entire disclosure of which is hereby incorporated by reference into this specification. Claim 1 of this patent describes “An improved process to make gemcitabine hydrochloride, the improvement consisting essentially of making the lactone intermediate, 2-deoxy-2,2-difluoro-D-erythro-pentafuranose-1-ulose-3,5-dibenzoate: [Figure] from D-erythro-2-Deoxy-2,2-difluoro-4,5-O-(1-ethylpropyl)-idene) pentoic acid tert-Butyl ester wherein, the D-erythro-2-Deoxy-2,2-difluoro-4,5-O-(1-ethylpropyl)-idene) pentoic acid tert-Butyl ester is prepared by the process of reacting S-tert-butyl difluoroethane thioate with 2,3-O( 1-ethylpropylidene)-D-glyceraldehyde, in a solvent and in the presence of a strong base; with the proviso that the process is conducted in the absence of a catalyst and in the absence of a silyl containing.”

As is known to those skilled in the human, biological organisms have built in “check points” which allow them to effectuate synchronization of cell growth upon the occurrence of various events. Thus, and referring to Chapter 17 of Bruce Alberts et al.'s “Molecular Biology of the Cell,” Fourth Edition (Garland Publishing, New York, N.Y.), it is disclosed that “We can illustrate the importance of an adjustable cell-cycle control system by extending our washing machine analogy. The control system of simple embryonic cell cycles, like the controller in a simple washing machine, is based on a clock. The clock is unaffected by the events it regulates and will progress through the whole sequence of events even if one of those events has not been successfully completed. In contrast, the control system of most cell cycles (and sophisticated washing machines) is responsive to information received back from the processes it is controlling. Sensors, for example, detect the completion of DNA synthesis (or the successful filling of the washtub), and, if some malfunction prevents the successful completion of this process, signals are sent to the control system to delay progression to the next phase. These delays provide time for the machinery to be repaired and also prevent the disaster that might result if the cycle progressed prematurely to the next stage.”

The Alberts et al. work also discloses that “In most cells there are several points in the cell cycle, called checkpoints, at which the cycle can be arrested if previous events have not been completed (FIG. 17-14). Entry into mitosis is prevented, for example, when DNA replication is not complete, and chromosome separation in mitosis is delayed if some chromosomes are not properly attached to the mitotic spindle . . . . Progression through G1 and G2 is delayed by braking mechanisms if the DNA in the chromosomes is damaged by radiation or chemicals. Delays at these DNA damage checkpoints provide time for the damaged DNA to be repaired, after which the cell-cycle brakes are released and progress resumes.”

The Alberts et al. work also discloses that “Checkpoints are important in another way as well. They are points in the cell cycle at which the control system can be regulated by extracellular signals from other cells. These signalswhich can either promote or inhibit cell proliferationtend to act by regulating progression through a G1 checkpoint, using mechanisms.”

As will be apparent from many of the aforementioned United States patents, one may utilize externally applied chemotherapeutic agents to synchronize the cells within a biological organism at a certain stage. Thus, e.g., reference may again be had to U.S. Pat. No. 6,511,818, the entire disclosure of which is hereby incorporated by reference into this specification.

In column 1 of U.S. Pat. No. 6,511,818, it is disclosed that “Precise coordination of the S and M phases of the eukaryotic cell cycle is critical not only for normal cell division, but also for effective growth arrest under conditions of stress. When damaged, a cell must communicate signals to both the mitotic and DNA synthesis machineries so that a mitotic block is not followed by an extra S phase, or vice versa. The biochemical mechanisms regulating this coordination, termed checkpoints, have been identified in lower eukaryotes, but are largely unknown in mammalian cells 1-3.” The references cited in this section of the patent include A. W. Murray, Nature 359, 599-604, 1992; P. Nurse, Cell 79, 547-550, 1994, and L. H. Hartwell et al., Science 266, 1821-1828, 1994.

As is also disclosed in column 1 of such United States patent, “DNA-damaging agents are used in the clinic to preferentially kill cancer cells. However, there is a need in the art to discover additional therapeutic agents which are selectively toxic to cancer cells.”

U.S. Pat. No. 6,511,818 describes and claims “1.1. A method of screening for potential anti-tumor agents, comprising the steps of: determining viability of homozygous p53 gene-defective human colonic cells incubated in the presence and in the absence of a test compound; and identifying the test compound as a potential anti-tumor agent if it causes cell death in the homozygous p53 gene-defective human colonic cells.”

Other United States patents also describe how to identify agents that synchronize cells at specific portions of the cell cycle. Reference may be had, e.g., to U.S. Pat. No. 5,879,889 (cancer drug screen based on cell cycle uncoupling), U.S. Pat. No. 5,882,865 (cancer drug screen based on cell cycle uncoupling), U.S. Pat. No. 5,888,735 (cancer drug screen based on cell cycle uncoupling), and U.S. Pat. No. 5,879,999 (cancer drug screen based on cell cycle uncoupling). The entire disclosure of each of these United States patents is hereby incorporated by reference into this specification.

In one preferred embodiment, a drug is used in such step 261 to synchronize the cells in the orgnanism at the M phase (metaphase), also known as “mitosis.” As is known, mitosis is the divison of the nucleus of euraryotic cells which occurs in four stages designated prophase, metaphase, anaphase, and telophase. In one aspect of this embodiment, the drug used in such step 261 synchronizes the cells in prophase. In one aspect of this embodiment, the drug used in such step 261 synchronizes the cells in metaphase. In one aspect of this embodiment, the drug used in such step 261 synchronizes the cells in anaphase. In one aspect of this emboidiemnt, the drug used in such step 261 synchronizes the cells in telophase.

In one embodiment, it is preferred that the drug used in step 261 stabilize the cells in metaphase. As is known to those skilled in the art, metaphase is the second stage in mitosis, during which the chromosomes arrange themselves in an equatorial region.

In another embodiment, it is preferred that the drug used in step 261 stabilize the cells in the “S Phase.” As is also disclosed in Chapter 17 of the aforementioned Alberts et al. text, “Replication of the nuclear DNA usually occupies only a portion of interphase, called the S. phase of the cell cycle . . . . The interval between the completion of mitosis and the beginning of DNA synthesis is called the G1 phase.” Reference also may be had, e.g., to U.S. Pat. No. 4,812,394 (flow cytometric measurement of DNA and incorporated nucleoside analogs), U.S. Pat. No. 5,633,945 (accuracy in cell mitosis analysis), U.S. Pat. No. 5,866,338 (cell cycle checkpoint genes), U.S. Pat. No. 6,172,194 (ARF-p19, a novel regulator of the mammalian cell cycle), U.S. Pat. No. 6,274,576 (method of dynamic retardation of cell cycle kinetics to potentiate cell damage), U.S. Pat. No. 6,455,593 (method of dynamic retardation of cell cycle kinetics to potentiate cell damage), and the like. The entire disclosure of each of these United States patents is hereby incorporated by reference into this specification.

As used in this specification, the term “synchronized” means that at least about 30 weight percent of the cells in question are in the desired phase, and preferably, at least about 50 weight percent of the cells in question are in the desired phase. In one embodiment, at least about 70 weight percent of the cells are in the desired phase.

One may determine the extent to which a collection of cells is synchronized by standard flow cytometry techniques. Thus, and referring to U.S. Pat. No. 4,812,394, one may utilize a process wherein, as described by claim 1, there is “1. A non-radioactive method for measuring unaltered cellular DNA and incorporated nucleoside analog, the method comprising the steps of: growing a population of cells in the presence of a non-radioactive predetermined compound, the non-radioactive predetermined compound being capable of assimilation into the DNA of the cells of the population to form an incorporated nucleoside analog whose presence can be detected by an immunochemical stain; altering a portion of the DNA of each cell of the population to substantially the same extent such that a first portion comprising altered DNA is formed and a second portion comprising unaltered DNA remains, the first portion being sufficiently large so that nucleoside analogs incorporated therein can be detected by an immunochemical stain specific for the incorporated nucleoside analog, and the second portion being sufficiently large so that G1 phase cells of the population can be distinguished from the G2 M phase cells of the population by a second signal generated by a second stain specific for the second portion; applying the immunochemical stain to the cells; applying the second stain to the cells; and detecting at substantially the same time and for each cell of a substantial portion of the population, a non-radioactive first signal from the immunochemical stain bound to the incorporated nucleoside analog in the first portion of DNA of each cell and a second signal from the second stain bound to the second portion of DNA of the same cell such that a first signal and a second signal are associated with each said cell of the substantial portion of the population.” As is disclosed in the specification of this patent, “A broad range of biological and biomedical investigations depends on the ability to distinguish cells that synthesize DNA from those that do not. Oncologists, for example, have devoted substantial effort to establishing correlations between the proportion of human tumor cells synthesizing DNA and treatment prognosis, e.g. Hart et al., Cancer, Vol. 39, pgs. 1603-1617 (1977). Effort has also been devoted to improvement of anticancer therapy with S-phase specific agents by treating when the experimentally determined proportion of tumor cells in S phase is maximal, e.g. Barranco et al., Cancer Research, Vol. 42, pgs. 2894-2898 (1982). In these studies, S-phase cells are usually assumed to be those that appear labeled in autoradiographs prepared immediately after pulse labeling with tritiated thymidine, or those with S-phase DNA content in DNA distributions measured flow cytometrically. Cancer researchers and oncologists have relied heavily on measurements of the proportion of DNA synthesizing cells to determine the cell cycle traverse characteristics of normal and malignant cells. The classical “fraction of labeled mitosis” procedure, Quastler et al., Experimental Cell Research, Vol. 17, pgs. 420-429 (1959), for example, depends on assessment of the frequency of mitotic cells that appear radioactively labeled in autoradiographs of samples taken periodically after labeling with tritiated thymidine. Studies of the cell cycle traverse characteristics of drug-treated cell populations typically require measurement of the amount of tritiated thymidine incorporated by cells in S phase (e.g., by liquid scintillation spectrometry) or determination of the fraction of cells with S-phase DNA content (e.g., by DNA distribution analysis), or both, Pallavicini et al., Cancer Research, Vol. 42, pgs. 3125-3131 (1982). Studies of mutagen-induced genetic damage that use unscheduled DNA synthesis as an index of damage also rely on the detection of low levels of incorporation of tritiated thymidine, e.g. Painter et al., Biochim. Biophys. Acta, vol. 418, pgs. 146-153 (1976).”

As will apparent, one may use other analytical techniques to determine the degree to which the cells are synchronized in a specified phase. In one embodiment, the phase-sensitive flow cytometer described in U.S. Pat. No. 5,270,548 is used; the entire disclosure of this United States patent is hereby incorporated by reference into this specification. This patent claims “1. A phase-sensitive flow cytometer for resolving fluorescence emissions from fluorochrome labeled cells into two components, comprising: flow cytometer means for providing a flow steam containing said labeled cells; an excitation light for exciting said labeled cells to fluoresce in said flow stream; modulation means for modulating said excitation light and generating a reference signal at a selected modulation frequency; detector means for receiving fluorescence emission spectra from said labeled cells as a modulated fluorescence signal and outputting a modulated intensity signal functionally related to said fluorescence emission spectra from said labeled cells; and phase detector means for resolving said modulated intensity signal into two signal components, each functionally related to a different fluorescence decay lifetime of said fluorescent emission spectra.”

Referring again to FIG. 5, and in the preferred embodiment depicted therein, it is preferred to treat the cells with the synchronizing agent for at least about 25 minutes prior to it is contacted with ultrasound in step 266. It is more preferred to wait at least about 60 minutes prior to time one contacts the cells with ultrasound. In one embodiment, one waits at least about 4 hours until after first administration of the synchronizing agent until the cells are contacted with ultrasound. In one embodiment, a period of at at least about 48 hours is allowed to pass from the initial administration of the synchronizing agent before the cells so synchronized are contacted with the ultrasound energy.

Referring again to FIG. 5, and in step 262 of this process, microtubules in diseased cells are preferably stabilized by one or more conventional means. As is known to those skilled in the art, stabilization of microtubles at metaphase can result in the synchronization of a population of cells at the metaphase checkpoint of the cell division cycle.

Thus, e.g., one may effectuate such stabilization by using anti-mitotic or other chemical agents known to affect microtubules, or using chemicals that influence proteins that aid in the stabilization of microtubules (e.g. Rho or FAK), or a process of post-translational modification to the tubulin protein, until the half-life of an individual microtubule in the mitotic spindle of a dividing cell is an average of at least 8 minutes, or more than 10 percent of the microtubules in a non-dividing cell have a half-life of more than 8 minutes. One may use standard means for stabilizing the microtubules to this extent. Thus, e.g., reference may be had to U.S. Pat. No. 5,808,898 (method of stabilizing microtubules); U.S. Pat. Nos. 5,616,608; 6,403,635; 6,414,015 (laulimalide microtubule stabilizing agents); U.S. Pat. Nos. 6,429,232; 6,500,859 (method for treating atherosclerosis or restenosis using microtubule stabilizing agent); U.S. Pat. No. 6,660,767 (coumarin compounds as microtubules stabilizing agents); U.S. Pat. No. 6,740,751 (methods and compositions for stabilizing microtubules and intermediate filaments); and the like. The entire disclosure of each of these United States patents is hereby incorporated by reference into this specification.

In step 264 of this process, the resonant frequency of the stabilized microtubules in the diseased cells to be treated is determined. As used herein, the term “resonant frequency” is that frequency which, at a power level of 10 miliwatts per square centimeter, a temperature of 37 degrees Celsius, and atmospheric pressure, is sufficient to break at least 50 weight percent of the microtubules in the cell after an exposure time of five (5) minutes. That frequency which breaks the maximum number of microtubules under these conditions is the resonant frequency.

In step 264 of the process depicted in FIG. 5, an estimate of the energy and wavelengths associated with the vibration of microtubules from an external source is conducted. By way of illustration and not limitation, and without being bound to any particular theory, applicants believe that such an estimate may be readily made in accordance with the discussion and the equations presented elsewhere in this specification.

A Theoretical Approach to Estimate the Type of Ultrasound to be Used in the Process 260

Without wishing to be bound to any particular theory, it is believed that the critical force required to break a microtubule can be calculated by the equation: F_{c}˜1/L², which indicates that the critical force is proportional to 1 divided by the square of L. L is the length of the microtubule.

An estimate of the F_{c} required to buckle a microtubule can be had from the experimentally derived values of flexural rigidity measured for microtubules. For the purposes of this example, and not wishing to be bound to this value, we will assign L to the value of 10 micrometers (μm) and F_{c} to the value of 6 pN.

Again, for the purposes of this example, without wanting to be bound to a single value, the flexural rigidity of the non-taxol stabilized microtubule can be described with the equation: EI=10.10⁻²⁴ Nm². For comparison purposes, actin's critical stress can be described for the purposes of this example: σ_{c}=5 dyne/cm²=0.5 N/m².

Although not wanting to be bound to this value outside of this example, the buckling pressure of a microtubule has been experimentally determined to be 240 dyne/cm².

The cross sectional area of a hollow tube is described, as in Johnathan Howard's Mechanics of Motor Proteins and the Cytoskeleton (Sinauer Press, 2001), on page 101 to be: A=(π/4)(d₂²−d₁²)=5×10⁻¹⁶ m². This equation, in which A represents area, can be applied to microtubules as they are a polymer in the shape of a cylinder and the values of d₂ and d₁, in the case of a microtubule, are simply the outer and inner diameters of the cyliner (25 nm and 15 nm, respectively).

Critical force (F_{c}) can be calculated based on this area in the equation: F_{c}=P_{c}×A=0.4 pN, in which P_{c} represents the critical pressure applied perpendicularly to the cross-sectional area A.

Young's modulus (Y) is a description of the stiffness of a material. Young's modulus for microtubules has been experimentally determined. Y=10⁹ N/m².

The spring constant (k) for a microtubule can be calculated from the Young modulus as given below: k=(A×Y)/L, in which A is the area of the cylindrical cross-section (described above), Y is the Young's modulus and L is the length of the microtubule, therefore: k=(π(25²−15²)×10⁹)/10=˜4 N/m.

This value is important because it is greater than the force of attraction between 2 protofilaments in a microtubule structure (2 N/m).

In general, one should use the formula (see the book by Jonathon Howard) to derive the formula for the critical force: F_{c}=π²(EI/L²) and thus calculate the propagation velocity for a standing vibrational wave in a microtubule by way of the following equation: υ=(F/ρ_{L})^{1/2} in which ρ_{L} is the linear mass density of the protein filament (microtubule) and F stands for the tension force that is less or at best equal to the critical force for breaking a microtubule.

One can then calculate the frequency of the vibrational mode according to: f=v/l=(F/ρ_{L})^{1/2}/l, where l is the wavelength of the standing wave. The fundamental harmonic will have the wavelength l=2L where L is the length of the microtubule cylinder along its axis. In general, the n-th harmonic will have the wavelength given by the formula: lₙ=2L/n. Hence, its frequency is given by: fₙ=nf, where f stands for the fundamental harmonic. The formula above is applied for the calculation of the fundamental harmonic, second harmonic, or third harmonic, etc by choosing the value of n as 1,2,3, etc . . . . For purposes of this example, EI is assigned to be 26×10⁻²⁴ Nm² in its native state while attached at both ends (one to a polar body, the other to a chromosome, as in mitosis). This value increases to 32×10⁻²⁴ Nm² when the microtubule is stabilized with taxol. Using this value, we can estimate the frequency to be in the range of 270-420 kHz for the fundamental harmonic with a second harmonic at twice the frequency to be in the range of 540-840 kHz, etc.

It should be noted that the frequency formula depends inversely proportionally to the length of a given microtubule. In this connection, polar microtubules are almost twice as long as kinetochore microtubules and hence, in order to break them by means of applying high frequency ultrasound, different frequency ranges must be selected (approximately half the values of those applied to break kinetochore microtubules). In general, this application of ultrasound for breaking up the mitotic apparatus in dividing cells requires a prior microscopic observation and analysis of the cell's cytoskeletal apparatus with particular attention to the length of the microtubules to be determined as accurately as possible. Having determined the lengths and elastic constants for all kinetochore and polar microtubules, a weighted superposition of the fundamental and first harmonic ultrasound modes must be calculated and then generated with a subsequent application to the cellular targets.

The mass density of tubulin is estimated to be approximately 900 kg/m³ while that of the surrounding medium (mainly water) is assumed to be 1000 kg/m³. The linear mass density of a microtubule cylinder is calculated assuming the length L, the outer and inner diameters d₂ and d₁, respectively, as stated above. Aqueous environment is filling the inner diameter region of the cylinder as well as forming a thin layer of bound water surrounding the outer surface. We assumed that a 3 angstrom layer of bound water is attached. With these assumptions, the linear mass density (mass per length) of a microtubule is approximately 5×10⁻¹³ kg/m. Using the formula for v stated above as a function of the force of tension applied to a microtubule (at most 6 pN) and the above linear mass density, we evaluate the propagation velocity of standing vibrational waves on microtubules to be in the range of 3-4 n/s which is much less than the propagation velocity of ultrasound in an aqueous medium (on the order of 1000 m/s).

The following is an estimate of the ultrasound intensity required to deliver a sufficiently strong amount of energy to break microtubules. The formula for the power delivered per cross-sectional area for a wave traveling at a speed v in a medium of mass density rho and having an amplitude A is given by: Power/Area=A² v f² rho, where f is the frequency of the wave. Estimating the amplitude A to be in the 3 angstrom range, the frequency in the MHz range and the velocity of propagation as well as mass density as given above, we obtain an estimate of the intensity as 0.1 W/m². However, this is only the power deposited in the form of microtubule oscillations. Since the ultrasound propagates at a much faster velocity in the medium before it is resonantly absorbed by the microtubules, the actual power generated at the source most be scaled up by the velocity ratio factor, i.e. we expect it to be at least in the range of 10-30 W/m² which corresponds to the 130-135 dB range on the decibel scale.

It is known that Taxol, and Taxol-type compounds, stabilize microtubules, prevent them from shortening and dividing the cell as a result of their shortening as they segregate the genetic material in chromosomes. Furthermore, Taxol increases the rigidity of microtubules making them susceptible to breaking given the right physical stimuli.

Ultrasound induces mechanical vibrations of microtubules. At the right frequency, and at the right power level, the application of ultrasound will cause the microtubules to first buckle and then break up.

The ultrasound used in the process of this invention preferably has a frequency of from about 50 megahertz to about 2 Gigahertz, and more preferably has a frequency of from about 100 megahertz to about 1 Gigahertz. The power of such ultrasound is preferably at least about 0.01 watts per square meter and, more preferably, at least about 10 watts per square meter. The ultrasound is preferably focused on the tumor to be treated. One may use any conventional means for focusing the ultrasound. Thus, e.g., one may use one or more of the devices disclosed in U.S. Pat. No. 6,613,0055 (systems and methods for steering a focused ultrasound array), U.S. Pat. Nos. 6,613,004, 6,595,934 (skin rejuvenation using high intensity focused ultrasound), U.S. Pat. No. 6,543,272 (calibrating a focused ultrasound array), U.S. Pat. No. 6,506,154 (phased array focused ultrasound system), U.S. Pat. No. 6,488,639 (high intensity focused ultrasound treatment apparatus), U.S. Pat. No. 6,451,013 (tonsil reduction using high intensity focused ultrasound to form an ablated tissue area), U.S. Pat. No. 6,432,067 (medical procedures using high-intensity focused ultrasound), U.S. Pat. No. 6,425,867 (noise-free real time ultrasonic imaging of a treatment site undergoing high intensity focused ultrasound therapy), and the like. The entire disclosure of each of these patent applications is hereby incorporated by reference into this specification.

In one embodiment, Taxol (or a similar composition) is delivered to the patient and, as is its wont, makes the microtubules more rigid. Thereafter, when the microtubules are polymerized in a dividing cell and substantially immobilized, the ultrasound is selectively delivered to the microtubules in the tumor, thereby breaking such microtubules and halting the process of cell growth and division, ultimately leading to cell death (apoptosis).

In one aspect of this embodiment, after the Taxol (or similar material) has been delivered to the patient, a high intensity magnetic field is applied to the tumor in order to selectively cause the Taxol to bind the microtubules in the tumor. Thereafter, the ultrasound is applied to break the microtubules so bound to the Taxol enhancing the efficacy of the drug due to a combined effect of the magnetic field, ultrasound and chemotherapeutic action of Taxol itself.

When microtubules have been broken, they tend to reform. Therefore, in one embodiment, and referring again to FIG. 5, the ultrasound is periodically or continuously delivered to the tumor synchronized to the typical time elapsed between subsequent cell division processes during which microtubules are polymerized (see, e.g., steps 261/270/272 of FIG. 5).

In one embodiment, a portable device is worn by the patient and applied to the tumor site; and this device periodically and/or continuously delivers ultrasound and/or magnetic energy to the patient. In one aspect of this embodiment, the device first delivers high intensity magnetic energy, and then it delivers the ultrasound energy.

Referring again to FIG. 5, and to the preferred embodiment depicted therein, in step 265 one can determine the harmonic frequencies that correspond to the resonant frequency determined in step 264. One may use a first harmonic of such resonant frequency, a second harmonic of such resonant frequency, and, in fact, any harmonic of the resonant frequency. As is known to those skilled in the art, a harmonic is one of a series of sounds, each of which has a frequency that is ain integral multiple of some fundamental frequency.

One may apply the resonant frequency to the stabilized microtubules and/or one of the harmonic frequencies, and/or a second of the harmonic frequencies and/or a third of the harmonic frequencies and/or a fourth of the harmonic frequencies and/or a fifth of the harmonic frequencies, etc. These frequencies may be applied simultanoueously, and/or they may be applied sequentially. One may alternate this application of frequency or frequencies with the administration of one or more stabilizing agents and/or synchronizing agents and/or antimototic agents and/or cytotacitc agents.

In this process, and in step 266 thereof, one may use any of the means for generating and focusing ultrasound energy that are known to those skilled in the art. Thus, e.g., one may use the ultrasound generator disclosed in U.S. Pat. No. 6,685,639, the entire disclosure of which is hereby incorporated by reference into this specification. This patent claims: “A high intensity focused ultrasound system, comprising: a controllable power supply; a B-mode ultrasound scanner; a therapeutic bed having a through hole; a liquid bag placed in the through hole and having opposite upper and lower portions, the lower portion of the liquid bag being attached to a combined probe, whereby a body portion of a patient lying immediately above the through hole may be scanned and treated by said system; and the combined probe comprising: a therapeutic head coupled to said controllable power supply for generating and focusing a ultrasound beam on a focal region at a temperature greater than 70 degrees centigrade, said therapeutic head comprising a ultrasound lens and piezoelectric ceramics coupled to said controllable power supply and disposed beneath the ultrasound lens, and an imaging probe coupled to said B-mode ultrasound scanner and mounted on a central axis of said therapeutic head so that the focal region of said therapeutic head is fixed at a predetermined location on a scanning plane; wherein said liquid bag contains vacuum degassed water having an acoustic impedance similar to that of human tissue, the upper portion of said liquid bag including an opening exposing said vacuum degassed water, said opening being open to an upper surface of said therapeutic bed so as said vacuum degassed water is adapted to be placed in direct contact with the skin of the patient's body portion; said system further comprising a multi-dimensional motional apparatus, on which the combined probe is mounted and which is moveable along three-dimensional rectangular coordinate axes and rotatable about one or two rotational coordinate axes, for driving said combined probe, said multidimensional motional apparatus includes a plurality of one-dimensional motional devices each being configured to either translate or rotate said combined probe in a specific direction.”

By way of yet further illustration, and not limitation, one may use one or more of the ultrasound generators described in U.S. Pat. No. 3,735,756 (duplex ultrasound generator); U.S. Pat. No. 4,718,421 (ultrasound generator); U.S. Pat. No. 4,957,100 (ultrasound generator and emitter); U.S. Pat. No. 4,976,255 (extracorporeal lithotripsy using shock waves and therapeutic ultrasound); U.S. Pat. Nos. 5,102,534; 5,184,065 (therapeutic ultrasound generator); U.S. Pat. No. 5,443,069 (therapeutic ultrasound applicator for the urogenital region); U.S. Pat. No. 6,270,342; and the like. The entire disclosure of each of these United States patents is hereby incorporated by reference into this specification.

By way of further illustration, one may also use the ultrasound generator disclosed in an article by article by I. Hrazdira et al., “Ultrasonicallly inducted altrations of cultured tumour cells,” European Journal of Ultrasound 8: 43-49, 1998. At page 45 of this article, is it disclosed that: “A laboratory generator operating at a frequency of 0.8 MHz was used as the source of continuous ultrasound.”

Without wishing to be bound to any particular theory, applicants' believe that the resonant freqency will will vary with the square root of the average length of the microtubules in the cells being treated. They also believe that the microtubules in diseased cells do not necessarily have the same length as the microtubules in non-diseased cells. It is believed, e.g., that cancer cells have microtubules that are up to about 10 percent longer than the microtubules of comparable non-cancer cells. Thus, by applying frequencies that are specific for the microtubules in the diseased cells, they preferentially treat the diseased cells with the process of this invention. Moreover, for the ultrasound application to be most effective in reaking up tumor cell microtubules, an apprpriate superposition of frequencies must be applied in correspondence to the lengths and rigidities of microtubules targetted.

Referring again to FIG. 5, and to step 264 thereof, a series of experiments may be preferably conducted with ultrasound waves with a power level of 10 milliwatts per square centimeter and different frequencies, at temperature of 37 degrees Celsius, and atmospheric pressure, and then the breakage of microtubules caused by such exposure is determined. That frequency which breaks the maximum number of microtubules is the resonant frequency. as will be apparent, the results of these experiments may be used to corroborate the estimates made by mathematical means of the resonant frequency of the stabilized microtubules. Alternatively, they may be used independently to determine the resonant frequency of the microtubules.

One may determine the extent to which any particular ultrasound wave breaks microtubules by conventional means. Thus, e.g., one may use the means described in the afrorementioned article by I. Hrazdira et al. (“Ultrasonically induced alterations of cultured tumor cells,” European Journal of Ultrasound 8 [1998], 43-49), in section 2.3 thereof. As is disclosed in such article, “For visualization of cytoskeleton components, an indirect immunofluorescence method was used. The cells in the monolayer were washed with phosphate buffer before adding 0.1% Triton for stabilization of membrane permeability. The cells were subsequently fixed by means of 3% paraformaldeyde. After fixation, secondary antibodies were added for 45 min . . . for microtubules . . . . Between each operation, the cells were washed by PBS. Finally, samples for fluorescene microscopy were prepared . . . . A total of 20 microphotographs of each controal and experimental sample were evaluated anonymously . . . . Changes in cytoskeletal structre were evaluated quantitatively . . . .”

Referring again to FIG. 5, and in step 266 of the process, the stabilized microtubules are then contacted with ultrasound energy.

In one embodiment, the frequency of the ultrasound energy is approximately the resonant frequency, plus or minus about ten percent. In one aspect of this embodiment, the frequency of the ultrasound energy is approximately the resonant frequency, plus or minus about 5 percent. In general, such frequency will often be in the range of from about 100 kilohertz to about 500 kilohertz. and, more preferably, from about 110 to about 200 kilohertz. In yet antoher embodiment, such frequency is from about 130 to about 170 kilohertz.

The power used for such exposure is preferably from about 1 to about 30 milliwatts per square centimeter and, more preferably, from about 5 to about 15 milliwatts per square centimeters.

At page 46 of the aforementioned Hrazdira et al. article, it was disclosed that “The disassembly of cytoskeleton components was not permanent. According to the time interval between sonication and cell fixation, a partial (at higher intensities) or total (at lower intensitivies) recovery of the cytoskeleton took place.” At page 49 of the Hradzdira et articles, it was disclosed that “We did not find any changes in the cells that could be entirely attributed to ultrasound action only. From the point of view of cytoskeletal alterations, ultrasound has to be considered as a non-specific stress factor.”

To help insure that applicants' process is more effective in causing permanent changes in the cell, an in step 268, the ultrasound excitation of the stabilized microtubules is ceased when the temperature of such microtubules reaches a specified temperature such as, e.g., a temperature of 70 degrees Celsius.

U.S. Pat. No. 6,685,639, the entire disclosure of which is hereby incorporated by reference into this specification, describes and claims “a high intensity focused ultrasound system for scanning and treating tumor” which creates a very high temperature (in excess of 70 degrees Celsisus) in the area of the “focal region.” As is disclosed in column 3 of this patent, “By means of focusing, the sytem causes ultrasonic waves to form a space-point with high energy (focal region); the energy of the region reaches over 1000 W/M² and the temperature instaneously rises to greater than 70 degrees centigrade . . . .”

Applicants wish to avoid prolonged exposure of the cells of living organisms to a temperature in excess of a specified temperature, such as, e.g., 42 degrees Celisus. Thus, when the temperature of the microtubules reaches such specified temperature, and in step 268, the process of ultrasound excitation is repeated.

Thereafter, in step 270, step 266 (the contacting of the stabilized microtubules with ultrasound energy) is repeated until the temperature of the microtubules reaches the aforementioned maximum temperature, at which point step 268 is repeated (in step 272). The cycle is continued for as many times as is necessary to induce apoptosis.

In one embodiment, step 266 is conducted for from about 1 to about 5 minutes, the microtubules are allowed to cool, and then such step 266 is repeated again and again.

Nuclear Localization Sequences

U.S. Pat. No. 6,495,518, the entire disclosure of which is hereby incorporated by reference into this specification, describes the addition of “peptide localization sequences.” This patent, which is entitled “Method for importing biologically active molecules into cells, discloses that: “Peptides have been developed for many therapeutic uses. For example, diseases currently targeted by new peptide drugs include heart conditions, cancers, endocrine disorders, neurological defects, respiratory conditions, allergies and autoimmune diseases. Although the manufacture of known therapeutic peptides can be achieved by known methods, i.e., classic synthetic techniques or recombinant genetic engineering, delivery of the peptides into a cell has remained problematic, since they cannot readily cross biological membranes to enter cells. Thus, current methods include permeabilization of the cell membrane, or microinjection into the cell. Both of these methods have serious drawbacks. Permeabilization of cells, e.g., by saponin, bacterial toxins, calcium phosphate, electroporation, etc., can only be practically useful for ex vivo methods, and these methods cause damage to the cells. Microinjection requires highly skilled technicians (thus limiting its use to a laboratory setting), it physically damages the cells, and it has only limited applications as it cannot be used to treat for example, a mass of cells or an entire tissue, because one cannot feasibly inject large numbers of cells.”

U.S. Pat. No. 6,495,518 also discloses that: “Similarly, delivery of nucleic acids has been problematic. Methods currently employed include the permeabilization described above, with the above-described drawbacks, as well as vector-based delivery, such as with viral vectors, and liposome-mediated delivery. However, viral vectors can present additional risks to a patient, and liposome techniques have not achieved satisfactorily high levels of delivery into cells.”

U.S. Pat. No. 6,495,518 also discloses that “Signal peptide sequences . . . which share the common motif of hydrophobicity, mediate translocation of most intracellular secretory proteins across mammalian endoplasmic reticulum (ER) and prokaryotic plasma membranes through the putative protein-conducting channels.2-11 Alternative models for secretory protein transport also support a role for the signal sequence in targeting proteins to membranes . . . . Several types of signal sequence-mediated inside-out membrane translocation pathways have been proposed. The major model implies that the proteins are transported across membranes through a hydrophilic protein conducting channel formed by a number of membrane proteins.2-11 In eukaryotes, newly synthesized proteins in the cytoplasm are targeted to the ER membrane by signal sequences that are recognized generally by the signal recognition particle (SRP) and its ER membrane receptors. This targeting step is followed by the actual transfer of protein across the ER membrane and out of the cell through the putative protein-conducting channel (for recent reviews, see references 2-5). In bacteria, the transport of most proteins across the cytoplasmic membrane also requires a similar protein-conducting channel.7-11 On the other hand, signal peptides can interact strongly with lipids, supporting the proposal that the transport of some secretory proteins across cellular membranes may occur directly through the lipid bilayer in the absence of any proteinaceous channels . . . .”

U.S. Pat. No. 6,495,518 also discloses that “Thus, though many attempts have been made to develop effective methods for importing biologically active molecules into cells, both in vivo and in vitro, none has proved to be entirely satisfactory.” The solution to this problem, presented in claim 1 of the patent, is: “A method of importing a nuclear localization sequence of NF-.kappa.B into a cell in a subject, comprising administering a cyclic peptide consisting essentially of . . . to the subject, wherein said cyclic peptide is imported into a cell in the subject.”

The process described in U.S. Pat. No. 6,495,518 may be used in conjunction with one or more of the therapeutic agents described elsewhere in this disclosure. In particular, such process may be used in conjunction with the nuclear localization sequence (NLS) which directs a moiety, to which it is attached, to the nucleus of the cell. The NLS is a short peptide usually, (but not limited to) 4 to 8 amino acid residues usually, but not limited to, highly charged species such as lysine or arginine, which can be covalently bound to the therapeutic molecule or other chemical of interest.

Nuclear localization sequences are well known to those skilled in the art. Thus, by way of illustration, reference may be had to U.S. Pat. No. 6,521,456, the entire disclosure of which is hereby incorporated by reference into this specification. This patent is enitled “Cellular transport system for the transfer of a nucleic acid through the nuclear envelope and methods thereof,” it discloses a method to use NLSs to transport transgenic nucleic acid molecules to the nucleus, and it claims “a nuclear transport agent for transferring a nucleic acid from cytoplasm into a nucleus of a eukaryotic cell comprising a first module and a second module, wherein the first module is module A that binds specifically to a DNA molecule so as not to form complexes consisting of more than one DNA molecule, and wherein the second module is module B that comprises an extended nuclear localization signal having a charge thus preventing the second module from mediating nonspecific binding of the nuclear transport agent to the DNA molecule.”

By way of yet further illustration, nuclear localization signals are described in U.S. Pat. Nos. 5,576,201; 5,580,766; 5,670,347; 5,712,379; 5,736,392; 5,770,581; 5, 5,783,420; 5,795,587; 882,837; 5,891,718; 5,973,116; 5,994,512; 6,033,856; 6,057,101; 6,106,825; 6,159,691; 6,165,720; 6.203,968; 6,222,095; 6,235,521 (phage bonded to nuclear location signal); U.S. Pat. Nos. 6,235,526; 6,297,253; 6,300,120 (phage with nuclear localization signal); U.S. Pat. Nos. 6,333,127; 6,372,720; 6,379,927; 6,465,246; 6,472,176; 6,476,296; 6,479,284; 6,521,456; 6,576,758; 6,586,240; 6,649,797; 6,664,368; 6,720,310; 6,746,868; 6,759,231 (phage with nuclear localization signal); U.S. Pat. Nos. 6,770,477; 6,777,544; and the like. The entire disclosure of each of thee United States patents is hereby incorporated by reference into this specification.

By way of yet further illustration, a database of nuclear localization signals is available at http://cubic.bioc.columbia.edu/db/NLSdb/ in which these 114 experimentally derived NLSs are described by their peptide sequence in single letter amino acid code: [de][kr]rr[kr][fyw], [de][rk]{2,4}[ga]r[pl][ga], [de][rk]{3,}?x[kr]{2,}?[pl], [de][st][pl]kr[stc], [de]k[nif]rr[dek][stmnq], [de]kk[pl][gl]k[gl], [de]kr[mqn]r[mqn]r, [de]kxrrk[mnq], [de]rkrr[deplq], [de]rxkkkk, [de]r{2,4}xrk[pl], [ed]r{4,}?[ed], [ga][kr]krx[kr][ga], [ga]kxkkk[mnq], [ga]rx[rk]x[rk][rk]x[qm], [gaplv]rkrkkr, [kar]tpiqkhwrptyltegppvkirietgewe[ka], [kr][de][kr][de]xx[kr]{4,}?, [kr][kr][kr][kr][kr][kr][kr], [kr][kr][qmn]r[rk][qmn]r, [kr][kr]x[kr][kr][kr]x[kr][kr], [kr]g{2,}?xxg{3,}?[rk], [kr]krkk, [kr]xxknkx{6,8}k[kr], [kr]{2,3}xxkr[kr][qlm], [kr]{2,}?[pl]x{1,4}[kr]{2,}?x{1,5}k{3,}?, [kr]{2}x{0,1}[kr]{2,4}x{25,34}k{2,4}x{1,2}k, [kr]{4}x{20,24}k{1,4}xk, [If][stk][viqm][kr]r[qmvi][stk]l, [mi]vwsrd[heq]rrk, [pl][kr]{5,7}[pl], [pl][pl][kr]r[de][kr][qst], [pl][rk][rk][dep]r[rk][fyw], [pl][rk][rk][kr][gapl][rk][stqm], [pl][rk]{2,3}k[pli][rk]x[pli]xk, [pl]kxxkrr, [pl]r[de]k[de]r, [pl]rkrk[pl], [pl]xxkr[iv]k[pl][de], [plq][kr]x{3,4}kkrk, [plq]k[rk]x{1,2}[rk]x{3,6}[rk][rk]x{1,2}[rk]x{1,2}[rk][rk], [plqmkr]r[kr][qm][kr]rxk, [plqmnkr]k[kr][kr]rxk[plqmnkr], [plv]k[rk]x[qmn][rk]r, [plv]k[rk]x[rk][rk][rk][pl], [plv]rk[st]r[de]k, [pvli][rk][rk][rk][rk][rk][qmn]k, [ql]k{2,4}x{8,12}[rk][ql][rk][ql]kr, [ql]xkrxkxkk, [qmn]r[rk]xkx[rk][rk], [rk][pliv][kr][rk]{2,4}[plvi]r, [rk]h[rk]xxx[rk]{2,4}xr, [rk]k{2,4}x[rk][ql][rk][pl], [rk]r[ms]kxk[kr], [rk]x[rk]x[kr]x{4,6}rkk, [rk]{2,4}x{1,2}[rk]x{0,2}[rk]x{3,}[rk]x{0,2}[rk][rk]{2,4}[pl], [rk]{2,4}x{2,4}[qlm][rk]x{2,3}[rk]kr, [rk]{3,}?x[rk]x[rk]x{4,9}[rk]{3,}?, [rk]{3,}?x{8,16}[rk]{4,}?, [rk]{4,}?[qmnpl][rk]x{3,4}[rk]{2}, [st]gx{1,3}g{3,}?x{1,2}g{3,}?[st], [stqm]rkrk[stqm], [stqm]rkrr[stqm], [stqm]rrrk[stqm], [ts][rk]kk[vli]r[pl], [yfw]rrrr[pl], apkrksgvskc, aptkrkgs, ckrkttnadrrka, cygskntgakkrkidda , d[kr]x{0,1}[ql][rk]{2,3}r, dk[ql]kk[ql], dr[mn]kkkke, eedgpqkkkrrl, eylsrkgklel, gggx{3}knrrx{6}rggm, gkkkyklkh, gkkrska, gr[rk]{2,4}xx[rk][ql], grkrkkrt, g {2,4}[rk]x{1,3}g{3}, hkkkkirtsptfttpktlrlrrqpkyprksaprrnkldhy, hrieekrkrtyetfksi, hrkyeaprhx{6}prkr, ikyfkkfpkd, k[ga]k[ag]kk[ag], k[ivqm]rr[vi][stk]l, k[kr][kr]rr[kr], k[kr][qmn][rk]r[qmn]r, k[mnq]rr[plvi]k[pl], k[pl]k{2,3}x{1,3}[rk]{2,4}x{6,9}k[kr], k[pl]k{3,}?xkk, k[plmn]rrk[mnq], k[rk]{2,4}[st]h, k[rk]{2, }?[ql]x{3,8}r{3}, k[rk]{3,5}x{11,18}[rk]kx{2,3}k, kakrqr, kdcvinkhhrnrcqycrlqr, khlkgr, khrkhpg, kk[mnqstc]r[mnqstc]k[mnqstc], and kkekkkskk.

In one preferred embodiment, a small nuclear localization signal (such as RKRKK) is covalently attached to carbon 10 of paclitaxel molecule. In another preferred embodiment, the taxane molecule is attached to the NLS by a short linker which is composed of a ribonucleic acid/deoxyribineucleic acid hybrid linker which would be cleaved in the nucleus by Rnase H or other types of linkers sensitive to other enzymatic activity such that the taxane molecule is released from the NLS and allowed to bind to the tubulin molecules there in. Although not wanting to be bound to any particular theory, it appears that these systems exploit the fact that tubulin type beta II is found inside the nuclear membrane of cancer cells and not normal cells, thereby allowing NLS-guided tubulin binding drugs to find therapeutic target proteins only in cancer cells.

In the following compound, 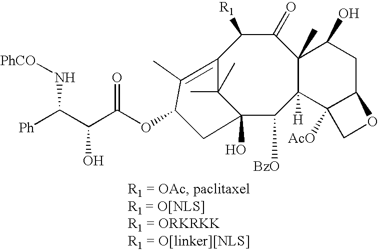
In the embodiment depicted above, when R₁ is OAc, the compound is paclitaxel. In one embodiment, a nuclear localization signal is linked to C10 wherein R₁ is O[NLS]. In another such embodiment, the nuclear localization signal is RKRKK, wherein R₁ is ORKRKK.

As described above, in yet another embodiment, there is employed a a linker molecule, wherein R₁ is O[linker][NLS]. In another embodiment the linker is nucleic acid, and the NLS is selected from the list presented above. Similar functional groups may be installed at other carbon positions around the taxane ring. For example, an NLS functional group may be installed at C4, C7, C9, and/or C10. In another embodiment, a plurality of NLS functional groups are present in a single taxane molecule. Yet other variations upon this theme will be apparent to those skilled in the art.

Metallized Microtubules

In this section of the specification, metallized mircotubules are discussed. These microtubles may be made by well known means, including the means disclosed in U.S. Pat. No. 5,650,787. The entire disclosure of this United States patent is hereby incorporated by reference into this specification.

As is disclosed in U.S. Pat. No. 5,650,787:” . . . a process for the deposition of thin metal coatings onto the microtubules is described in Schnur et al., “Lipid-based Tubule Microstructures”, Thin Solid Films, vol. 152, 1987, pages 181-206. Microtubules with metal coatings such as nickel or permalloy can be aligned with either an electric or a magnetic field during the formation of the anisotropic solid polymer composite.”

Reference also may be had to U.S. Pat. Nos. 6,280,759 and 5,492,696, the entire disclosure of each of which is hereby incorporated by reference into this specification. As is disclosed in U.S. Pat. No. 6,280,759: “As described in U.S. Pat. No. 3,318,697, it is known to metal coat lipid and wax globules. For pharmaceutical and other purposes, it is known to incorporate materials inside a waxy globule or a liposome. It is further known that polymerizable phospholipids form hollow cylindrical structures which are commonly referred to as tubules. These are described in U.S. Pat. Nos. 4,877,501 and 4,990,291. The efficient synthesis of these compounds is fully described in U.S. Pat. No. 4,867,917 entitled “Method for Synthesis of Diacetylenic Compounds”. The methods necessary to coat these microstructures with a range of metals is fully described in U.S. Pat. No. 4,911,981 entitled “Metal Clad Lipid Microstructures” These tubules are hollow tube-shaped microstructures fabricated by self organization of polymerizable diacetylenic phospholipid molecules. Morphologically, tubules are analogous to soda straws with diameters of approximately 0.05 to 0.7 μm and lengths from 1 to 1,000 μm. The tubule diameter, the length and the number of bilayers comprising the wall are all controllable parameters which are controlled by the fabrication methods employed. The preparation of tubules is also discussed in an article by Schnur et al., “Lipid-based Tubule Microstructures”, Thin Solid Films, 152, pp. 181-206, (1987) and the articles cited therein. That same article, in which one of the inventors is a co-author, also describes metal coating tubules and using them as microvials to entrap, transport and deliver polymeric reagents to a desired site.”

By way of yet further illustration, one may metallize microtubules by the process described by R. Kirsch et al. entitled “Three-dimensional metallization of microtubules” that paws published in “Thin Solid Films,” 305 (1997), pages 248-253.

As is disclosed on page 248 of the Kirsch et al. reference, “In order to deposit an adherent, thin metal film onto protein template surfaces, we followed the method of electroless metal plating developed by Brenner and Riddell . . . for finishing material surfaces. Electroless deposition occurs by a redox process, where the cation of the metal to be deposited is chemically reduced.” The Brener and Riddell article cited was by A. Brenner et al., “Proc. Am. Electroplaters Soc. 33 (1946) 16; 34 (1947) 156.

The Kirsch et al. article also discloses (on page 248) that “Electroless deposition occurs by a redox process, where the cation of the mtal to be deposited is chemically reduced. The redox process of electroless deposition takes place only on appropriate catalystic surfaces. Therefore, a noncatalytic substrate, such as the surface of a nonconductor, must be treated with a noble metal catalyst [3] before it can be metallized by an electroless process.” The reference “3” cited in this portion of the Kirsch et al. article was to an article by F. Pearlstein, Met. Finish. 53 (1955) 59.

The Kirsch et al. article also discloses (on page 248) that “The first biomolecular template based tubular microtubules were fabricated utilizing phospholipids tubles [5]. Markowitz et al. [6] found that the diameters of metallized lipid tubules depend upon the duration of dialysis carried out prior to metallization. They observed a distrubiton of diameters ranging from 100 to 900 nm.” The reference “5” cited was an article by J. M. Schnur et al. appearing in Thin Solid Films 152 (1987)181. The Markowitz et al. reference (“6”) was published in Thin Solid Films 224 (1993) 242.

Metallization of proteinaceus tubules was first demonstrated by Pazirendeh et al. [7] using rhapidosomes as the templates. Rhapidosomes are found in certain bacteria. They have a well defined diameter of 25 nm. and an average length of ˜500 nm.” The Pazirendeh et al. reference “7” was published in Biometrics 1 (1992) 41.

The Kirsch et al. article (at page 249) discussed the electroless metal plating of microtubules (MTs), and stated state “MTs are cytoskeletal protein polymers. They form highly dynamic structures which may polymerize and depolymerize during their function, e.g., they form transport tracks for organelles in the cell and deermine mainly cellular architecture. These are interesting features for artificial nanoassembly. MTs are tubular protein filaments. Each tubules if formed by longitudinally arranged protofilaments, each about 4-5 nm in diameter. The protofilaments consit of about 8 nm long heterodimers polymerized head to tail. The heterodimers comprise α- and β-subuits (MW about 50 kDa each). The outer diameter of the MTs is 25 nm, the same as that of rahpidosomes, and is also well defined.”

The Kirsch et al. article, at page 249, also discloses that “MTs have the advantage that they can be assembled in vitro to a length of several micrometers. On the other han d, both the process of self-assembly and the morphological stability of MTs are very sensitive to the chemical environment and to temperature. For example, MTs cannot withstand treatment in strong alkaline or acidic solutions nor tmepetarues about 60 degrees C., as are commonly applied in electroless copper plating baths . . . . We show here that these problems can be circumvented by carrying out electroless plating of MTs under conditions similar to those required for the assembly process, i.e at a pH of about 7 and physiological temperatrures. In a first step, the protein surface is activated by direct adsorption of molecular palladium catalysts (first demonstrated by Chow et al. [9] for rhapidosomes.” The cited Chow et al. reference waspublished in Nanostruct. Mater. 2 (1993) 495.

The Kirsch et al. article also discloses (at page 249) that “In a second step, under both appropriate chemical conditions and temperatures, nickel is deposited onto activated MTs by applying electroless metallization baths based on dimethylamine borane as the reducing agent, as developed by Narcus [10] and Paunovic [11].” The cited references “10” and “11” were published by H. Narcus (Electronics Symp. Plating 54 [1967] 380, and by M. Paunovic (Plat. Surf. Finish. 70 [1983] 62), respectively.

In the experiments described in the Kirsch et al. paper, “The MTs were isolated from porcine brain by three cycles of temperature-dependent disassembly/reassembly [12]”; the cited reference “12” was an article by M. L. Shelanski et al. published in Proc. Nat. Acad. Sci. USA 70 (1973) 765.

The Kircsh et al. paper then disclosed that “Pure tubulin heterodimer preparations were obtained by phosphocellulose column chromatography [13]. All experiments in ths study started from a tubulin heterodimer preparation stored at −80 degres C. in a buffer solution of 100 mM MES (2-morpholino-ethanesulfonic acid monohydrate), 1 mM EGTA (ethylene glycol bis-(β-aminoethyl)-tetra-acetic acid), and 0.5 nM MgCl₂. The protein concentration was about 1 mg ml⁻¹. The MTs were assembled in vitro by adding 0.25 nM GTP (guanosin-5′-triphosphate) and 10 nM taxol . . . and warming the sample to 37 degrees C. The MT formation was accompanied by turbidity measurements at 240 nm wavelength. The steady state level at which the tubulin mass in the polymerized state shows no further increase, was usually observed after 10 min. Thereafter, the polymer solution was centrifuged for 30 min. at 14500 g to separate the MTs from the unpolymerized tubulin. The supernatant was discarded and replaced by the same volume of pure MES buffer at pH 6.4 and the pellet was resuspended.”

Section 2.2 of the Kircsh et al. reference, appearing on page 249 thereof, discloses a process for the activation and nickel plating of the microtubule surface. It is disclosed that “To activate the MT surface by adsorptiojn of Pd catalyst particles, a volume of about 300 μl of the assembled MT solution was treated with an equal volume of a fresh saturated Pd (CH₃COO)₂ solution for about 2 h at room temperature (pH 6.2). The catalyzed MTs were then washed with MES buffer by ultrafitration using a 300 kDa MW cut-off membrane filter. The pellet in the membrane filter was subsequently redispesed in about 500 μl of MES buffer.”

The nickel plating process was then disclosed. It was stated that “For the nickel plating, we used two slightly different metallization baths, with dimethylamine borane (DMAB) as the reducing agent. The two baths wee prepared with analytical-grade reagents and deionized water. Electroless nickel ‘solution A’ [10] contained 50 g l⁻¹ Ni(CH₃COO)₂.6H₂O, 25 gl⁻¹ sodium citrate, 25 gl⁻¹ of 85% lactic acid aq. sol., and 25 gl⁻¹ of DMAB, whereas ‘solution B’ [11] contained 39.4 g l⁻¹ NiSO₄.6H₂O, 20 gl⁻¹ sodium citrate, 10 gl⁻¹ of 85% lactic acid aq. sol. and 4 gl⁻¹ DMAB. In both cases, the Ph was adjusted with NH₄OH.” The cited references “10” and “11,” which referred to the solutions “A” and “B”, were articles by H Narcus and M. Paunovic cited elsewhere in this specification.

The Kirsch et al. article then disclosed that “The Pd-activated MT preparation was mixed with an equal volume of the metallization bath. After 1 min, black metallized MTs settled at the bottom. The mtallization process was usually stopped by decreasing the concentration of the metallization bath by at least a factor of 100. The metallized MTs were then washed and stored in water.”

The metallized microtubules will have different electrical properties, depending upon the metal used. One advantage they possess is they may be used in a wet state (in a solution).

In one embodiment, the microtubules are metallized with a conductive metal, such as silver, ruthenium or copper. In another embodient, the microtubules are coated with palladium or gold or platinum.

Depending upon the configuration of the metal-plated tubules, and their interconnections, electrical assemblies comparable to diodes (rectifiers), transformers, transmitters, antennas, etc., may be formed, as will be discussed in more detail elsewhere in this specification.

In one embodiment, microtubules are removed from a cell, placed in a buffer solution, and stabilized with Taxol; they can be disposed, e.g., in a Petri dish.

The metallized or partially metallized microtubules can be connected to other microtubules, or other biological moities, by microtubule associated proteins (MAPS).

Microtubule assemblies may be used as sensors, for the properties of the microtubules vary upon exposure to different materials. Thus, e.g., bacteria interact with microtubules and affect their conductive properties. Thus, e.g., pH, magnesium ions, calcium ions, temperature, ultrasound, zinc ions, kinesins, etc. affect the properties of microtubules; and microtubules can be used to sense them or to sense a change in their condition.

Microtubules are only stable within a very narrow range of temperatures, typically between 7 and 37 degrees Celsius. Microtubules react to a pressure change. Mechanical stress applied to microtubules will affect their electrical properties.

Microtubules may be used as rectifiers. They are inherently anisotropic, being composed of alpha/beta tubulins each of which has a different value of the next electric charge at a particular ambient pH or salinity of the solution in which they are placed.

In one embodiment, microtubules are coated with layers of both conductive particles and and magnetic particles. One may make interconnections through these layers (via MAPs) and/or on these layers to form different circuits in the manner done with printed circuit boards.

In one embodiment, three-dimensional circuits comprised of metallized microtubles are prepared. These assemblies can be manipulated with local magnetic fields and/or oriented at various angles to form pre-designed circuitry.

As is known to those skilled in the art, whereas individual proteins are normally not considered to be good conductors of electricity, protein filaments can be conducting under specific conditions, especially when sufficiently hydrated. The latter effect has been shown to have a percolation threshold and microtubules often exhibit significant conductivity properties. Applicants have carried out preliminary computations of the conductivity coefficient for microtubules under various conditions of protofilament number and lattice structure predicting that under favorable conditions a 1-mm microtubule should have the conductivity of approximately 100,000 (Wm)\hat{ }−1 which is in the good intrinsic semiconductor range.

A Process for Preparing a Modified Microtubule Assembly

In this portion of the specification, a process for preparing a modified microtubule assembly will be described.

FIG. 6 is a schematic of a biological circuit 300 comprised of biological polymeric material 302 and a source 304 of alternating current.

Among the polymeric biological materials that may be used as biological material 302 are the microtubules described elsewhere in this specification. Alternatively, and in one embodiment, one may use the polymorphic tubulin assemblies described, e.g., in, an article by E. Unger et al, “Structural Diversity and Dynamics of Microtubules and Polymorphic Tubulin Assemblies,” Electron Miscrosc. Rev., Volume 3, pp. 355-395, 1990.

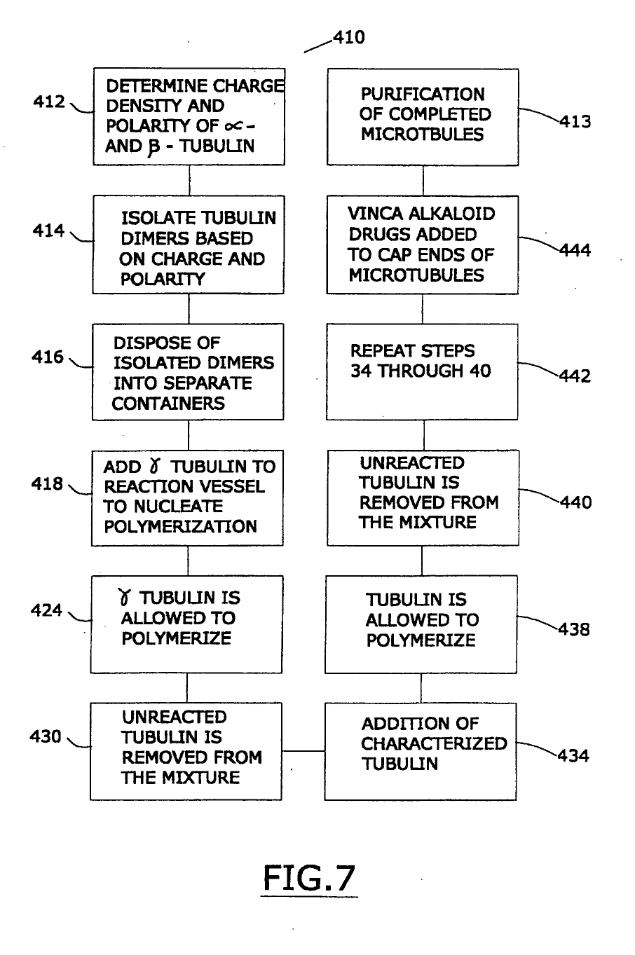

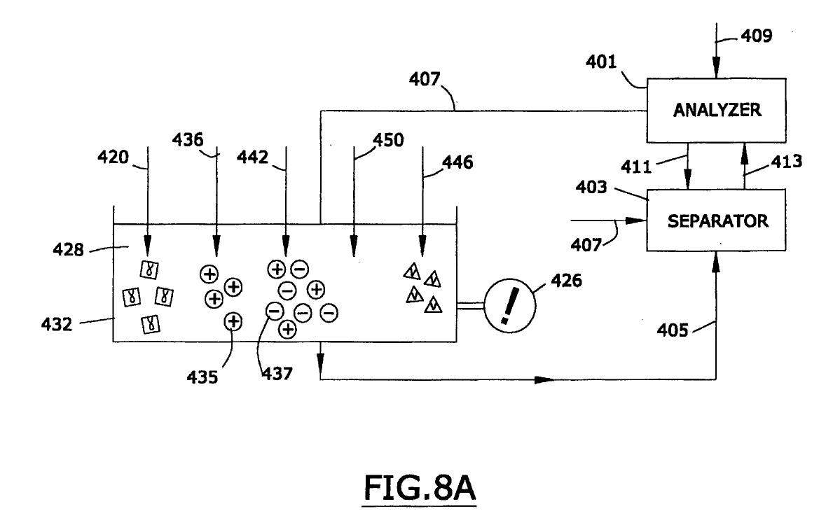

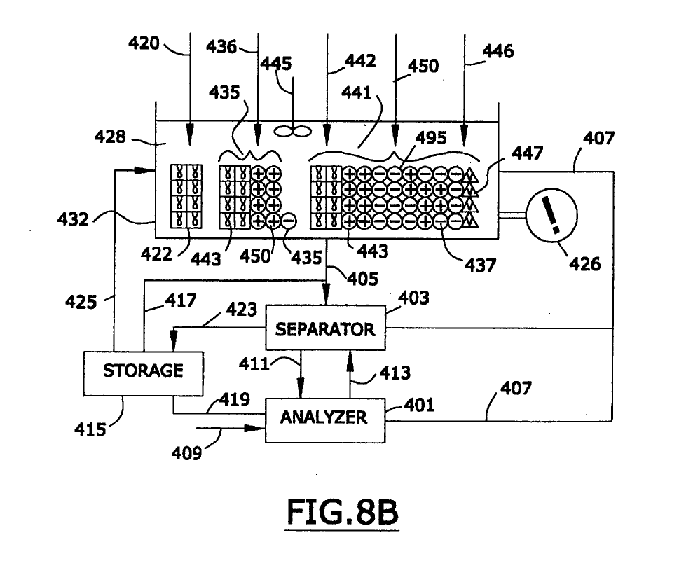

The biological materials used as material 302 may also be used as a reagent in the processes depicted in FIGS. 7, 8A, and 8B to make other biological materials to be used as material 302.

Referring again to the Unger et al. article, and in the abstract of the Unger et al. article, at page 355, it is disclosed that “Tubulin, the main protein of microtubules (MTs), has the potency of forming a variety of other assembly products in vitro: rings, ring-crystals, C- and S-shaped ribbons, 10 nm fiberes, hoops, sheets, heapted sheets, MT doublets, MT triplets, double-wall MTS, macrotubules, curled ribbons, and paracrystals. The supramoleuclar subunits of all of them are the protofilaments which might be arranged either parallel to the axis (e.g., in MTs, ribbons) or curved (e.g., in hoops, marcrotubules) . . . . All assembly products mentioned are described structurally . . . .” Each of these “other assembly products” may be used as the biological material 302. Alternatively, or additionally, each of these “other assembly products” may be used as a reagent in one or more of the processes depicted in FIGS. 7, 8A, and 8B in order to make even more “other assembly products” that may be used as either biological material 302 and/or biolelectronic material that may be used in one or more of the other processes and assemblies of this invention.

At page 365 of the Unger et al. article, tubulin assembly and disassembly is described. It is disclosed that “The main component of MTs is tubulin. This is a globular protein with a molecular mass of approximately 50 kDa . . . and 4 nm in diameter. The isoelectric point of both tubulins, for which numerous isotypes have been described (Lee et al., 1986) were found near 5.5, resulting above all from the relatively high content of acidic residues at the C-terminals . . . . ” Such tubulin, and/or any of its assembly products, may be used as biological material 302 and/or as a reagent in one or more of the processes of FIGS. 7, 8A, and 8B.

The Unger et al. article, at page 356, also discloses that “A feature of the tubulin dimers is their ability to form MTs by self-assembly in vitro at physiological temperatures in the presence of Mg²⁺ and GTP. On the other hand, in the cold MTs disassemble into dimers and some oligomers (see Section II.A).” Such tubulin dimers and oligomers may also be used as may be used as biological material 302 and/or as a reagent in one or more of the processes of FIGS. 7, 8A, and 8B.

Certain ring structures comprised of tubulin were described at pages 357 et seq. of the Unger et al. article. At page 357, it was disclosed that “MTP isolated from mammalian brain typically contains numerous ring or ring-like structures-single rings, double rings (consisting of two concentrically arranged rings with different diameters), triple rings (analogously constructed as the double rings), plane spirals, and further species . . . . Sometimes, two or more structures lie one on top of another. The outer diameter of ring assemblies ranges up to 57 nm.” Such ring structures may be used as biological material 302 and/or as a reagent in one or more of the processes of FIGS. 7, 8A, and 8B.

At page 358, the Unger et al. article disclosed that “The type of rings formed depends upon MTP composition and medium parameters, such as ionic strength, temperature, and pressure. Image reconstruction of electron micrographs . . . revealed 8, 12, and 16 α-dimers in triple rings and 12 and 16 in double rings . . . . Polycations can have a remarkable influence on the formation of rings. In the presence of polylysine, single rings appear (43 nm outer diameter, 6 nm radial thickness) whose inner side is found to bind 1 to β tubulin subunits . . . Histones, core histones or H1 cause the formation of unordered aggregates of (single) ring structures . . . .” Such polycations and/or histones and/or core histones and/or H1 may be used as a reagent in one or more of the processes of FIGS. 7, 8A, and 8B.

The formation of ringed crystal structures is also disclosed at page 358 of the Unger et al. article. It is stated that “Under certain conditions, double rings can form crystals, e.g., after long incubation of tubulin . . . with 15 nM Mg 2+at 0 degrees C. . . . or after 37 degrees C. inclubation of tubulin . . . with 1 mm ATP/5 MM Mg²/3.4 M glycerol . . . . The crystals c an be up to 100 μm in exent and several μm thick . . . .” Such ringed crystal structures may be used as biological material 302 and/or as a reagent in one or more of the processes of FIGS. 7, 8A, and 8B.

At page 383 of the Unger et al. article, the end-to-end annealing of microtubules was discussed. It was disclosed that “ . . . MTs assembled from chicken erythrocyte tubuln rapidly anneal end-to-end with MTs from brain tubulin. In a similar assay, Rothwell et al. (1987) used MTs from tyrosinolated and detyrosinolated tubulin. The annealing effect was also found in experiments of Caplow et al. (1986). Using mixtures of Tetrahymena axonemes and MTs, they demonstrated that the axonemene elongation is more rapid with a low concentration of long MTs at steady state than with a high number concentration of short MTs. The annealing phenomenon also acts in the presence of taxol, which strongly suppresses dissociaton events at the MT ends . . . .” Such long microtubules and/or such short microtubules may be used as biological material 302 and/or as a reagent in one or more of the processes of FIGS. 7, 8A, and 8B.

The formation of double-walled microtubules is discussed at pages 384-385 of the Unger et al. article. It is disclosed that “Double-wall MTs are not only formed by assembly of tubulin in the presence of certain polycations, they can be built up also by addition of polyc ations to preformed MTs . . . . Under favourable conditions (e.g. H1 excess) it is even possible to get a pure population of double-wall MTs from normal MTs . . . .” Such double-walled microtubules may be used as biological material 302 and/or as a reagent in one or more of the processes of FIGS. 7, 8A, and 8B.

It is also disclosed at page 385 of the Unger et al. article that “When H1 is added to tubulin sheets induced by Zn²⁺, besides mult-layered sheet aggregates, numerous curved sheets with a double-wall MT-like appearance were obserived.” Such H1 and/or zinc ions may be used as a reagent in one or more of the processes of FIGS. 7, 8A, and 8B.

The formation of macrotubules is also discussed at age 385 of the Unger et al. article. It is disclosed that “Macrotubules have been found as a result of MT disruption . . . . Recently we have demonstrated that macrotubules can arise from the outer wall of double-wall MTs upon the addition of tubulin . . . . Unlike macrotubules originating from direct conversions of MTs, these macrotubules have an inside-out orientation of wall protofilaments.” Such macrotubules may be used as biological material 302 and/or as a reagent in one or more of the processes of FIGS. 7, 8A, and 8B.

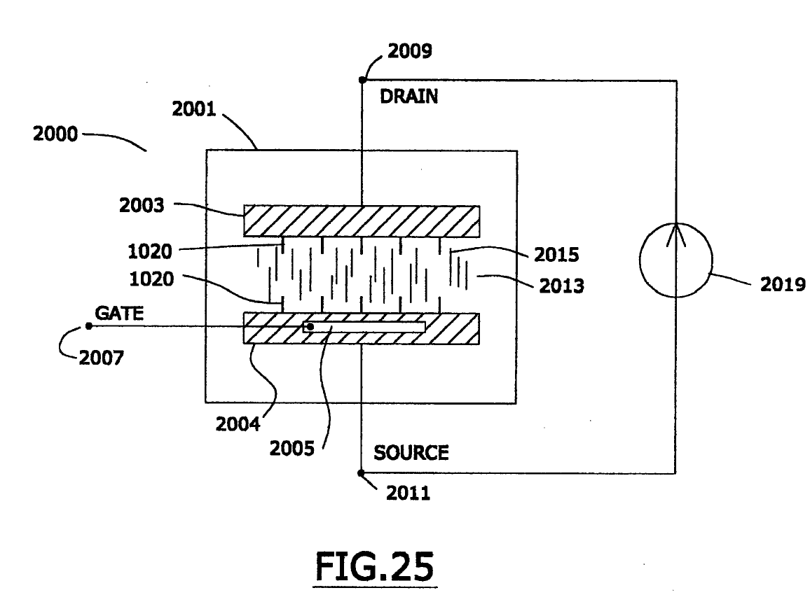

FIG. 25 of the Unger et al. article lists various polymorphic tubulin assemblies including dimmers, oligomers, rings, spirals, ring crystals, ring fragments, hoops, C-ribbons, sheets, heaped sheets, S-ribbon, an MT/ribbon complex, a double-wall MT, a macrotuble, a curled ring, and a paracrystal. Such polymorphic forms of tubulin may be used as biological material 302 and/or as a reagent in one or more of the processes of FIGS. 7, 8A, and 8B.

Another article that dealt with “aberrant forms of tubulin” was one by W. Vater et al. on “Tubulin Assembly in the Presence of Calcium Ions and Taxol: Microtubule Bundling and Fomration of Macrotubule—Ring Complexes,” Cell Motility and the Cytoskeleton 36:76-83 (1997). The abstract of this article disclosed that “ . . . assembly in the presence of Ca²⁺ and taxol leads to structural aberrations. The kind of aberration depends on the order of addition of taxol and Ca²⁺ to tubulin. When taxol was added first, microtubules were formed preferentially. But, these microtubules typically associated with each other by close wall-to-wall alignments or they formed complexes with some C-shaped protofilament ribbons, resulting in microtubule bundles or doublet- and triplet-like microtubule structures, respectively. When Ca² was added firt, macrotubules, rings, and ring crystals were the dominant assembly products. Mostly, the macrotubules were also bundled or they enclosed rings in their lumen.” Such calcium ions and/or such taxol and/or such aberrant forms of tubulin may be used as biological material 302 and/or as a reagent in one or more of the processes of FIGS. 7, 8A, and 8B.

At page 77 of the Vater et al. article, tubulin self-assembly is discussed. It is disclosed that “Tubulin, isolated and purified from cell homogenates, is able to self-assemble into MTs in vitro. This process requires certain conditions, among the appropriate concentrations of Mg²⁺ ions (Lee and Timaschef, 1977). The cited Lee et al. article, on “In vitro reconstitution of calf brain microtubules: Effects of solution variables”, was published in Biochemistry 16:1754-1764. Such magnesium ion and/or such “solution variables” may be used and/or adjusted in one or more of the processes of FIGS. 7, 8A, and 8B.

The Vater et al. article also discusses the effects of calcium ions, stating that “By contrast, Ca²⁺, like cold, usually causes MT disassembly leading to ring structures (circular protofilaments) and similar tubuln ologomers (Weisenberg, 1972).” The cited Weisenberg article, on “Microtubule formation in vitro in solutions containing low calcium concentrations,” was published in 1972 in Science 177:1104-1105. Such calcium ion and/or such cold may be used in one or more of the processes of FIGS. 7, 8A, and 8B.

It is also disclosed in the Vater et al. article that “ . . . Ca²⁺ ions are able to cause the formation of curled protofilament ribbons and macrotubules . . . ,” citing articles by Matsumarua et al, Langord, and Stromberg et al. The Matsumura et al. article, on “Polymorphism of tublin assembly. In vivo formation of sheet, twisted ribbon, and microtubule”, was published in 1976 in Biochim. Biophys. Acta 453:162-175. The Langford article, on “In vitro assembly of dogfish brain tubulin in the induction of coiled ribbon polymers by calcium,” was published in 1978 in Exp. Cell Res. 111: 139-151. The Stromberg et al. article, on “Differences in the effect of Ca²⁺ on isolated microtubules from cod and cow brain,” was published in 1994 in Cell Motil. Cytoskel. 28:59-68. Such coiled protofilament ribbons and/or such sheet microtubules and/or such twisted ribbon microtubules may be used as biological material 302 and/or as a reagent in one or more of the processes of FIGS. 7, 8A, and 8B.

In one preferred embodiment, the material 302 is an inorganic material that forms an inorganic microtubule. Such an inorganic microtubule is described, e.g., in U.S. Pat. No. 5,651,976, the entire disclosure of which is hereby incorporated by reference into this specification. This patent claims (in claim 1) “1. A composition for use in the delivery of an active agent at an effective rate for a selected time, comprising: hollow mineral microtubules selected from the group consisting of halloysite. cylindrite, boulangerite, and imogolite, wherein said microtubules have inner diameters ranging from about 200 .ANG. to about 2000 .ANG., and have lengths ranging from about 0.1 μm to about 2.0 μm, wherein said active agent is selected from the group consisting of pesticides, antibiotics, antihelmetics, antifouling compounds, dyes, enzymes, peptides. bacterial spores, fungi, hormones, and drugs and is contained within the lumen of said microtubules, and wherein outer and end surfaces of said microtubules are essentially free of said adsorbed active agent.” Such inorganic microtubule may be used as biological material 302 and/or as a reagent in one or more of the processes of FIGS. 7, 8A, and 8B.

In one preferred embodiment, the material 302 is a microtubule made from lipid material, such as that described in U.S. Pat. No. 6,013,206, the entire disclosure of which is hereby incorporated by reference into this specification. This patent claims (see claim 1) “1. A method of forming lipid microtubules, comprising the steps of: dissolving a lipid in a ethanol/ethanol/water solvent in which the vol % of methanol is about 50 to about 95 based on the total combined volume of methanol and ethanol, and the total combined vol % of methanol and ethanol is about 60 to about 90, based on the total volume of said methanol/ethanol/water solvent; allowing lipid microtubules to self-assemble in said methanol/ethanol/water solvent; and separating said formed lipid microtubules from said methanol/ethanol/water solvent.” In one aspect of this embodiment, the lipid microtubules thus formed are metallized. The process for metallizing such miroctubules is described at columns 5-6 of the patent, wherein it is disclosed that “The present invention also provides for a method to electrolessly plate the microtubules with a metallic coating to render them mechanically more robust and conductive. To achieve such a coating without breakage of the microtubules it is necessary to prevent the rapid evolution of hydrogen bubbles (a natural byproduct of the plating chemistry). Rapid evolution will cause pressure to build within the microtubules, thus bursting them. In addition large gas bubbles offer a surface attractive to the microtubules which then rise within the plating bath to aggregate and then become “welded” together by the plating process where they touch forming large aggregates that are difficult to redisperse.” Such lipid microtubule may be used as biological material 302 and/or as a reagent in one or more of the processes of FIGS. 7, 8A, and 8B.

As is also disclosed in U.S. Pat. No. 6,013,206, “The catalyzed microtubules are suspended in a large volume of water sufficient to produce a volume of 10× the original suspension volume of the naturally settled tubules. Following this step, the plating bath is added slowly, typically as follows. A solution of the plating bath is added to the dilute suspension such that the final concentration reaches about 5 to about 25% (typically about 10%) of that customarily used for plating surfaces. The standard dilution of a plating bath can vary depending on the commercial plating bath selected. For each plating bath selected, however, the manufacturer provides a standard (i.e., customary) plating bath dilution. If desired, about 0.025% by weight K-90 grade poly(vinylpyrollodone) (PVP) may be added to the bath to further reduce the possibility of cold welding and clumping of the high aspect ratio microstructures. If used, the poly(vinylpyrollodone) should first be reacted with a metal salts solution to prevent the PVP from stripping metal ions from the plating bath, thus having an adverse effect on the plating bath performance. Using the plating method described herein, however, the use of PVP is generally not needed to prevent cold welding and clumping.” Such catalyzed microtubules may be used as biological material 302 and/or as a reagent in one or more of the processes of FIGS. 7, 8A, and 8B.

As is also disclosed in U.S. Pat. No. 6,013,206, “Once the plating process has been observed to initiate, additional additions of plating bath are added so that the final concentration of the plating bath is reached after 9 further additions. When the reaction appears to subside a sample of the tubules are observed by microscopy to ensure that the coating is not less than 100 nm or meets process requirements. If the desired coating thickness has not yet been achieved, the plating bath is replenished to provide the aforementioned final concentration and plating is continued until the reaction again subsides. Obtaining a thickness of 100 nm or greater generally requires the addition of a total 6× or less of the recommended amount of plating solution for plating printed circuit boards. Serial addition of the plating solution maintains the desired low concentration of plating solution throughout the plating process. The use of an amount of plating bath greater than that required for plating to the desired thickness should be avoided, since excess metal salts would remain in solution following attainment of sufficient thickness. Following plating, the microtubules are either filtered from solution (preferred method) or allowed to settle and the excess bath drawn off. The plated tubules are then rinsed repeatedly with water until all plating salts have been removed. The tubules are then treated with a surface passivating agent, such as a suspension of a silane (e.g., hexamethyldisilizane), ethylene glycol, or a sugar to prevent undue oxidation.”

By way of further illustration, one may use the metallized microtubules referred to in U.S. Pat. No. 5,650,787, the entire disclosure of which is hereby incorporated by reference into this specification. Thus, e.g., as is disclosed at columns 4-5 of such patent, “Metallized microtubules, which are hollow tubule-shaped microstructures, are presently the preferred implementation within this category. The fabrication of these structures is described in Yager et al.,” Formation of Tubules by a Polymerizable Surfactant”, Molecular Crystals Liquid Crystals, vol. 106, 1984, pages 371-381, while a process for the deposition of thin metal coatings onto the microtubules is described in Schnur et al., “Lipid-based Tubule Microstructures”, Thin Solid Films, vol. 152, 1987, pages 181-206. Microtubules with metal coatings such as nickel or permalloy can be aligned with either an electric or a magnetic field during the formation of the anisotropic solid polymer composite.” Such microtubules with metal coatings may be used as biological material 302 and/or as a reagent in one or more of the processes of FIGS. 7, 8A, and 8B.

As is also disclosed in U.S. Pat. No. 5,650,787, “An experimental example of such an anisotropic solid core rod is one made with 0.2% (by weight) of nickel-coated microtubules dispersed in Optistik 2060, and aligned with a 1.5 kG magnetic field while cured (polymerized) with ultraviolet light for two hours. This was done in a 4 cm long Teflon tube (4.4 mm outside diameter, 3.35 mm inside diameter). The solid anisotropic rod composite was removed from the Teflon tube, placed in a millimeter wave (30 GHz) Mach-Zehnder interferometer, and its phase shift was measured as it was axially rotated in the rectangular waveguide. It showed a 60° phase shift when its anisotropic direction was rotated from parallel to perpendicular to the millimeter wave E-field. This corresponds to an effective birefringence of .DELTA.n=0.04, although the actual .DELTA.n of the rod is higher since the rod did not fill the rectangular waveguide cavity.”

By way of further illustration, and not limitation, one may use one or more other components of the cytoskeleton as those disclosed, e.g., in U.S. Pat. No. 6,699,969, the entire disclosure of which is hereby incorporated by reference into this specification. As is disclosed in column 1 of such patent, “The cytoskeleton constitutes a large family of proteins that are involved in many critical processes of biology, such as chromosome and cell division, cell motility and intracellular transport. Vale and Kreis, 1993, Guidebook to the Cytoskeletal and Motor Proteins New York: Oxford University Press; Alberts et al., (1994) Molecular Biology of the Cell, 788-858). Cytoskeletal proteins are found in all cells and are involved in the pathogenesis of a large range of clinical diseases. The cytoskeleton includes a collection of polymer proteins, microtubules, actin, intermediate filaments, and septins, as well as a wide variety of proteins that bind to these polymers (polymer-interacting proteins) Some of the polymer-interacting proteins are molecular motors (myosins, kinesins, dyneins) (Goldstein (1993) Ann. Rev. Genetics 27: 319-351; Mooseker and Cheney (1995) Annu. Rev. Cell Biol. 11: 633-675) that are essential for transporting material within cells (e.g., chromosomal movement during metaphase), for muscle contraction, and for cell migration. Other groups of proteins (e.g., vinculin, talin and alpha-actinin) link different filaments, connect the cytoskeleton to the plasma membrane, control the assembly and disassembly of the cytoskeletal polymers, and moderate the organization of the polymers within cells.” One or more of such other components of the cytoskeleton may be used as biological material 302 and/or as a reagent in one or more of the processes of FIGS. 7, 8A, and 8B.

Referring again to FIG. 6, they may be one or more protein filaments. As is disclosed in U.S. Pat. No. 5,882,881, “The cytoskeleton plays an important role in the growth, division, and migration of eukaryotic cells. Changes in cellular morphology, the repositioning of internal organelles, and cellular migration all depend on complex networks of protein filaments that traverse the cytoplasm. These protein filaments fall into three main categories according to their size: microtubules, intermediate filaments, and microfilaments. Both microtubules and microfilaments are made of globular subunits which can quickly polymerize and depolymerize in the cell resulting in movement and morphological changes. Intermediate filaments are made of fibrous protein subunits and tend to be more stable with longer half-lives than most microtubules and microfilaments.” A similar disclosure also appears in U.S. Pat. No. 5,789,189, the entire disclosure of which is hereby incorporated by reference into this specification. This latter patent discloses (in column 1) that “Current theory holds that cells have a pool of unpolymerized globular subunits in the cytoplasm which is used to rapidly form the cytoskeletal microtubules and microfilaments. Microtubules are formed by a dimer of tubulin proteins which take on a helical shape to form filaments. Similarly, microfilaments comprise actin proteins which agglutinate together to form elongated filaments. In addition to these fibers, the cytoskeleton is also made up of many other components for linking the filaments to each other or to the plasma membrane. Many cytoplasmic components can influence the rate of filament polymerization or depolymerization. Also, drugs have been discovered which affect the rate of filament polymerization and lead to either abnormal accumulations of protein filaments or unpolymerized globular subunits.” One or more of such protein filaments may be used as biological material 302 and/or as a reagent in one or more of the processes of FIGS. 7, 8A, and 8B.

By way of yet further illustration, one may use as polymeric material 302 actin filaments, macrotubules, carbohydrates, one or more tubulin heterodimers, and the like. One or more of such actin filaments may be used as biological material 302 and/or as a reagent in one or more of the processes of FIGS. 7, 8A, and 8B.

In one preferred embodiment, intermediate filaments are used as polymeric material 302. As is known to those skilled in the art, intermediate filaments are intracellular fibers having a diameter of about 8 to 12 naometers, which is between that of microfilaments and microtubules. Intermediate filaments are heterogeneous in their protein composition and are an important compoentn of the cytoskeleton. Reference may be had, e.g., to page 246 of J. Stensch's “Dictionary of Biochemstiry and Molecular Biology,” Second Edition (John Wiley & Sons, Inc., New York, N.Y., 1989). Reference also may be had, e.g., to U.S. Pat. Nos. 5,527,773; 6,296,850; 6,660,837; and the like. One or more of such intermediate filaments may be used as biological material 302 and/or as a reagent in one or more of the processes of FIGS. 7, 8A, and 8B.

In another embodiment, microfilaments are used as the polymeric material 302. As is disclosed at page 300 of the aforementioned Stensch et al. dictionary, microfilaments are thin, intracellular fibers having a diameter of about 5-8 nanometers and consisting essentially of actin. They exist in tow forms, lattice microfilaments (a losse network of short, interconnected filaments), and sheath microfilaments (bundles of fibers). Reference may be had, e.g., to U.S. Pat. Nos. 4,701,406; 5,789,189; 5,882,881; 6,074,659; 6,200,808; 6,376,525; and the like. The entire disclosure of each of these United States patents is hereby incorporated by reference into this specification.

Microfilaments are often referred to as “actin filaments,” and the latter term will be used in this specification. As is disclosed, e.g., on page 909 of B. Alberts et al.'s “Molecular Biology of The Cell,” Fourth Edition (Garalnd Science, New York, N.Y., 2002), “Actin filaments (also known as microfilaments) are two-stranded helical polymers of the protein actin. They appear as flexible structures, with a diameter of 5-9 nm, and they are organized into a variety of linear bundles, two-dimensional networks, and three-dimensional gels.” Reference also may be had, e.g., to U.S. Pat. Nos. 5,464,817; 5,656,589; 5.851,993; 6,331,659; 6,376,525; 6,403,766; 6,727,071; and the like. The entire disclosure of each of these United States patents is hereby incorporated by reference into this specification.

Referring again to FIG. 6, and in the preferred embodiment depicted therein, it will be seen that the polymeric material 302 is connected to a source 304 of alternating current. Other circuit elements typically present in circuit 300 have been omitted for the sake of simplicity of representation.

The source 300 of alternating current may be any source conventionally used such as, e.g., a pickup coil, a generator, an oscillator, household current, a radio-frequency signal, and the like.

In one embodiment, the inorganic microtubules are metallized.

FIG. 7 is a flow diagram of a preferred process 410 for preparing specified organic assemblies, and FIG. 8 is a schematic of a portion of such process 410. For the sake of illustration and not limitation, FIG. 7 describes the preparation of a tubulin assembly with a specified polarity and charge density. It will be apparent, however, that the process of FIG. 7 (and FIGS. 8A and 8B) may readily be used to prepare other biological assemblies with other specified properties.

Referring to FIG. 7, and in step p 412 of this process, and in one preferred embodiment, the polarity and charge density of various alpha and beta tubulins is determined. Some or all of these alpha- and beta-tubulins may thereafter be used, as desired, as a reagent in the process depicted n FIGS. 8A and 8B by adding and/or removing sauch alpha- and/or beta-tubulins from the reaction mixture 428 at specified times and/or by purifying and/or modifying such alpha- and/or beta-tubulins and thereafter using them in the reaction process.

As is disclosed in U.S. Pat. No. 6,750,330, the entire disclosure of which is hereby incorporated by reference into this specification, many different forms of tubulin and its monomeric precursors have been isolated. At lines 42 et seq. of column 1 of this patent, it is disclosed that “Different forms of tubulin have been isolated. These include a microtubule associated protein (MAP)-rich tubulin that is 50% to 97% purified (Shelanski, M. L., Gaskin, F., and C. R. Cantor, 1973, Proceedings of the National Academy of Sciences USA, 70, 765-768), highly purified (97% to 99.99% or apparently 100% purified by silver stain or coomassie-blue stained SDS-PAGE) tubulin, e.g., Phospho-cellulose purified tubulin (Lee, J. C., Tweedy, N., S. N. Timasheff, 1978, Biochemistry, 17(14), 2783-2790), tubulin from crude cancer cell line extracts (Weatherbee, J. A., Luftig, R. B., R. R. Weihing, 1980, Biochemistry, 19 (17), 4116-4123), tubulin isolated from higher eukaryotes and their cell lines (Weatherbee et al. 1980), tubulin isolated from fungi and yeasts and their cell lines (Davis, A., Sage, C. R., Dougherty, C., K. W. Farrell, 1993, Biochemistry, 32, 8823-8835), tubulin isolated from parasitic organisms or their cell lines (Dawson, P. J., Gutteridge, W. E., K. Gull, 1983, Molecular and Biochemical Parasitology, 7(3), 267-277), and tubulins isolated from recombinant systems and recombinant organisms (Davis, A., Sage, C. R., Dougherty, C., K. W. Farrell, 1994, Science, 264, 839-842.) Some or all of these “many forms of tubulin and its monomeric precursors” may be used in the processes of FIGS. 7, 8A, and/or 8B.

As is also disclosed in U.S. Pat. No. 6,750,330, “Tubulin is an essential intracellular protein that is necessary for mitosis, transport of intracellular material, cell structure, and cell motility. Tubulin is composed of a heterodimer of two closely related 55 Kilodalton proteins called alpha and beta tubulin. These two proteins are encoded by separate genes or small gene families, whose sequences are highly conserved throughout the eukaryotic kingdom.”

The polymerization of tubulin to form microtubles is discussed at lines 22-41 of column 1 of U.S. Pat. No. 6,750,330, wherein it is disclosed that “Tubulin polymerizes to form structures called microtubules. When tubulin polymerizes it initially forms protofilaments. Microtubules consist of 13 protofilaments and are 25 nm in diameter, each μm of microtubule length being composed of 1650 tubulin heterodimers. Microtubules are highly ordered fibers that have an intrinsic polarity. There is a dynamic flux between microtubules and tubulin. When this equilibrium is perturbed by anti-tubulin agents like paclitaxel (taxol), cells will arrest in mitosis and eventually die . . . .” Some or all of these 13 protofilaments may be used either as biological material 302 (see FIG. 6) and/or in one or more of the processes described in FIGS. 7, 8A, and/or 8B.

Referring again to FIG. 7, and in step 412 of FIG. 7, the charge density and polarity of “prior art” alpha- and beta-tubulins can be determined by reference to the “prior art” that describes such materials and its properties. One may, e.g., use the database described in this patent application.

Alternatively, or additionally, one may determine the polarity and charge density of various alpha- and beta-tubulins by electrophoresis. As is disclosed, e.g., at page 148 of J. Stensch's “Dictionary of Biochemistry and Molecular Biology,” Second Edition (John Wiley & Sons, Inc., New York, N.Y., 1989), electroporesis is “The movement of charged particles through a staionary liquid under the influence of an electric field Electrophoresis is a powerful tool for the separation of particles and for both preparative and analystical studies of macromolecules. The particles are separated primarily on the basis of theier charge and to a lesser extent on the basis of theis size and shape.” Reference may also be had, e.g., to U.S. Pat. No. 3,879,280 (gel slap electrophoresis cell), U.S. Pat. No. 5,399,255 (platform for conducting electrolphoresis), U.S. Pat. No. 5,562,813 (two dimensional electrophoresis apparatus), U.S. Pat. No. 5,589,104 (electrophoresis separation gel), U.S. Pat. No. 5,637,203 (platform for conducting electrophoresis), U.S. Pat. No. 6,533,913 (electrophoresis method, electrophoresis device, and marker sample used for the same), U.S. Pat. No. 6,572,746 (compositons for the rehydration of an electrophoresis support), U.S. Pat. No. 6,783,649 (high throughput capillary electrophoresis sytesm), U.S. Pat. No. 6,783,651 (system for pH-neutral stable electrophoresis gel), U.S. Pat. No. 6,793,790 (sample collection system for gel electrophoresis), U.S. Pat. No. 6,818,718 (electrophoresis gels), and the like. The entire disclosure of each of these United States patents is hereby incorporated by reference into this specification.

Referring again FIGS. 8A and 8B, and in the preferred embodiment depicted therein, the analyzer 401 and the separator 403 may be part of the same unit, such as an electrophoresis assembly, or they may be separate components. A sample may be removed from the reaction mixture 428 and/or separately charged via lines 405 and/or 407 and/or 409 and/or 411 and/or 413 to the analyzer 401 and/or the separator 403, and in one or both of these assemblies the sample may be separated into its component parts and/or analyzed. Thereafter, depending upon the properties found, one or more of the component parts of the sample and/or the sample itself may be added into reaction mixture 428 (via lines 403 and/or 407).

In one embodiment, best illustrated in FIG. 8B, a sample of the rection mixture 428 is removed thereform via line 405 and then fed to storage 415 via line 417. Alternatively, or additionally, material may be fed to storage 415 from either analyzer 401 (via line 419) and/or separator 403 (via line 423), and, at one or more selected times during the reaction process, material may be withdrawn from storage 415 via line 425 and fed into the reactin mixture 428.

Thus, by way of illustration and not limitation, a sample may be withdrawn from the reaction mixture 428 via line 405, it may be separated into various alpha-tubuln and beta-tubulin fractions in separator 403, and the charge density and the polarity of each such alpha-tubulin and/or beta-tubulin may be determined in analyzer 401.

One may use other means for determining the charge density and polarity of alpha-tubulins and beta-tubulins. Thus, e.g., one may use isoelectric focusing. This technique is discussed at page 254 of the aforementioned Stensch et al. reference, where it is described as “An electrophoretic technique for fractionating amphoteric molecules, particularly, proteins, that is based on their distrubiton in a pH gradient under the influence of an electric field that is applied across the gradient. The molecules distribute themselves in the gradient according to their isoelectric pH values. Positvely charged proteins are repelled by the anode and negatively charged proteins are repelled by the cathode: consequently, a given protein moves in the pH gradient and binds at a point where the pH of the gradient equals the isoelectric pH of the prtein. The pH gradient is produced in a chromatographic column by the electrolysis of amphoteric compounds and is stabilized by either a density gradient or a gel.” Reference also may be had, e.g., to U.S. Pat. No. 3,915,839 (apparatus for isolectric focusing), U.S. Pat. No. 3,951,777 (isoelectric focusing devices), U.S. Pat. No. 3,962,058 (flat bed isoelectric focusing devices), U.S. Pat. No. 4,204,929 (isoelectric focusing method), U.S. Pat. No. 4,312,739 (medium for isoelectric focusing), U.S. Pat. No. 4,362,612 (isoelectric focusing apparatus), U.S. Pat. No. 4,441,978 (separation of proteins using electrodialysis—isoelectric focusing combaintion), U.S. Pat. No. 4,481,141 (device for isoelectric focusing), U.S. Pat. No. 4,588,492 (rotating apparatus for isoelectric focusing), U.S. Pat. No. 4,670,119 (isoelectric focusing device and process), U.S. Pat. No. 4,673,483 (isoelectric focusing apparatus), U.S. Pat. No. 4,963,236 (apparatus and methods for isoelectric focusing), U.S. Pat. No. 4,971,670 (isoelectric focusing process and means for carrying out said process), U.S. Pat. No. 5,082,548 (isoelectric focusing apparatus), U.S. Pat. No. 5,376,249 (analysis utilizing isoelectric focusing), U.S. Pat. No. 5,468,359 (method of determining presence of an analyate by isoelectric focusing), U.S. Pat. No. 5,866,683 (isoelectric point markers for isoelectric focusing with fluorescence detection), U.S. Pat. No. 6,572,751 (method and apparatus for continous flow isoelectric focusing for purifying biological substances), U.S. Pat. No. 6,638,408 (method and device for separation of charged molecules by solution isoelectric focusing), and the like. The entire disclosure of each of these United States patents is hereby incorporated by reference into this specification.

Referring again to FIG. 7, and in step 414 thereof, individual tubulin dimers are isolated based upon their charge and their polarity. In step 416, the individual tubulin dimers so isolated are optionally disposed in separate containers (not shown). In one embodiment, the individual tubulin dimers so isolated are disposed in storage 415.

In addition to isolating individual tubulin dimers, or instead of isolating individual tubulin dimers, one may synthesize alpha- and/or beta-tubulns with specified charge polarities and densities. One may determine by conventional analyses whether any particular tubulin dimer or monomer is has a positive or negative charge polarity. Without wishing to be bound to any particular theory, applicants believe that the amount and polaritiy of charge in the tubulin moieties is a function, at least in part, of the types of amino acids that coprise such tubulin. Reference may be had, e.g., to page 28 of the Ph.D. thesis of Jonathan A. M. Brown, “A Study of the Interactions between Electromagnetic Fields and Microtubules . . . ,” Uiversity of Alberta, Emdonton, Canada, May 28, 1999. Referring to such page 28, and of the twenty-naturally occurring amino acids, aspartic acid, and glutamic acid have negative charges, and histidine, lysine, and arginine have positive charges. As will be apparent, one may modify the net charge in a tubulin moiety by replacing a uncharged amino acid with an amino acid of a specified charge; and vice versa. Additionally, or alternatively, acylation of the tubulin protein with other foreign compounds (such as fluorescent molecules) may affect the net charge.

The amino acid sequences of tubulin monomers may be determiend by standard tubulin databases. Thereafter, one may make approirate substitutions of amino acids to change the charge; and one then may construct the desired modified tubulin by means standard peptide synthesis. Reference may be had, e.g., to column 5 of U.S. Pat. No. 6,492,151, the entire disclosure of which is hereby incorporated by reference into this specification. As is disclosed in such column 5, “Because of the degeneracy of the genetic code, a large number of functionally identical nucleic acids encode any given protein. For instance, the codons GCA, GCC, GCG and GCT all encode the amino acid alanine. Thus, at every position where an alanine is specified by a codon, the codon can be altered to any of the corresponding codons described without altering the encoded polypeptide. Such nucleic acid variations are ‘silent variations,’ which are one species of conservatively modified variations. Every nucleic acid sequence herein which encodes a polypeptide also describes every possible silent variation of the nucleic acid. One of skill will recognize that each degenerate codon in a nucleic acid can be modified to yield a functionally identical molecule. Accordingly, each silent variation of a nucleic acid which encodes a polypeptide is implicit in each described sequence.”

U.S. Pat. No. 6,492,151 also discloses that “Also included within the definition of target proteins of the present invention are amino acid sequence variants of wild-type target proteins. These variants fall into one or more of three classes: substitutional, insertional or deletional variants. These variants ordinarily are prepared by site specific mutagenesis of nucleotides in the DNA encoding the target protein, using cassette or PCR mutagenesis or other techniques well known in the art, to produce DNA encoding the variant, and thereafter expressing the DNA in recombinant cell culture. Variant target protein fragments having up to about 100-150 amino acid residues may be prepared by in vitro synthesis using established-techniques. Amino acid sequence variants are characterized by the predetermined nature of the variation, a feature that sets them apart from naturally occurring allelic or interspecies variation of the target protein amino acid sequence. The variants typically exhibit the same qualitative biological activity as the naturally occurring analogue, although variants can also be selected which have modified characteristics.”

U.S. Pat. No. 6,492,151 also discloses that “Amino acid substitutions are typically of single residues; insertions usually will be on the order of from about 1 to about 20 amino acids, although considerably longer insertions may be tolerated. Deletions range from about 1 to about 20 residues, although in some cases, deletions may be much longer.”

U.S. Pat. No. 6,492,151 also discloses that “Substitutions, deletions, and insertions or any combinations thereof may be used to arrive at a final derivative. Generally, these changes are done on a few amino acids to minimize the alteration of the molecule. However, larger characteristics may be tolerated in certain circumstances.”

U.S. Pat. No. 6,492,151 also discloses that “The following six groups each contain amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W). (See, e.g., Creighton, Proteins (1984)).”

By way of further illustration of means for making “conservative amino acid substitutions,” reference may also be had to U.S. Pat. No. 6,492,158, the entire disclosure of which is also hereby incorporated by reference into this specification. As is disclosed at column 7 of this patent, “When percentage of sequence identity is used in reference to proteins or peptides, it is recognized that residue positions that are not identical often differ by conservative amino acid substitutions, where amino acid residues are substituted for other amino acid residues with similar chemical properties (e.g,. charge or hydrophobicity) and therefore do not change the functional properties of the molecule. Where sequences differ in conservative substitutions, the percent sequence identity may be adjusted upwards to correct for the conservative nature of the substitution. Means for making this adjustment are well known to those of skill in the art. The scoring of conservative substitutions can be calculated according to, e.g., the algorithm of Meyers & Millers, Computer Applic. Biol. Sci. 4:11-17 (1988), e.g., as implemented in the program PC/GENE (Intelligenetics, Mountain View, Calif.).”

Referring again to FIGS. 7 and 8B, the individual tubulin dimmers isolated in step 414, or synthesized by the appropriate amino acid substitution(s), may be lypholized to render them stable during storage; and they may be disposed within storage 415 (see FIG. 8B). As is known to those skilled in the art, lypholization is “The removal of water under vacuum from a frozen sample; a relatively gentle process for the removal of water in which the water sublimes from the solid to the gaseous state.” See, e.g., page 282 of the aforementioned Stensch dictionary. Reference may also be had, e.g., to claim 1 of U.S. Pat. No. 6,750,330 (the entire disclosure of which is hereby incorporated by reference into this specification), which describes “1. A method for the preparation of lyophilized active tubulin comprising the steps of; (a) running at least one cycle of polymerization and depolymerization of tubulin; (b) conducting differential sedimentation centrifugation to create a pellet of active tubulin; (c) re-suspending the pellet at tubulin concentration between 1 μg/ml and 200 mg/ml in a lyophilization solution comprising distilled water, 5% w/v sucrose, 1% w/v Ficoll, 15 mM Pipes at a pH of 6.9. 0.5 mM MgCl2, and 0.5 mM GTP; and (d) lyophilizing the re-suspended pellet.” In this patent, it is disclosed that “Tubulin can be isolated from most eukaryotic and some recombinant prokaryotic sources by standard methods. Various degrees of purity are produced by these methods. (Weisenberg and Timasheff, 1970; Weatherbee et al. 1980; Davis et al. 1993; Barnes, G., Louie, J. M., D. Botstein, 1992, Biochemistry, 32, 8823-8835; Lubega, G. W., Geary, T. G., Klein, R. D., R. K. Prichard, 1993, Mol. Biochem. Parasitol., 62, 281-292.) Tubulin is isolated as MAP-rich bovine brain tubulin (Shelanski et al. 1973) by three cycles of polymerization and depolymerization, greater than 99% purified bovine brain tubulin (phospho-cellulose purified tubulin by the method of Lee et al. 1978), DEAE-Cellulose purified Hela S3 Cell line tubulin (Weatherbee et al. 1980), and DEAE-Cellulose purified yeast and recombinant yeast tubulin (Davis et al. 1993). V.(2) Modified Forms of Tubulin.”

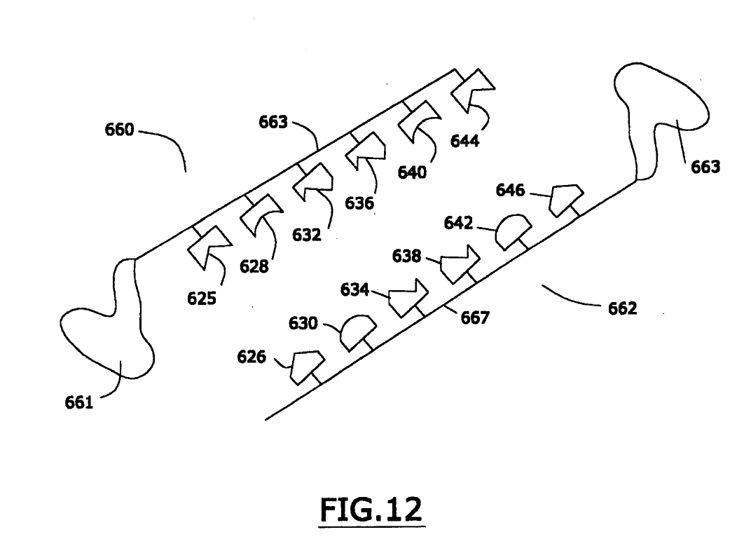

U.S. Pat. No. 6,750,330 also discloses that “There are currently several types of chemically and enzymatically modified tubulins reported in the literature. Modified tubulins include biotinylated, fluorescent, tyrosinylated, non-tyrosinylated, acetylated, caged fluorescence and fluorescent analog derivatives. The lyophilization procedure described in Section V.(3) below has been tested on biotinylated and fluorescent derivatives. These derivatives are made stable by the lyophilization process (See FIG. 12), and, thus, have an increased shelf life as compared to non-lyophilized samples. V.(3) Lyophilization Procedure.” Each of these “several types of chemically and enzymatically modified tubulins” may be used as a reagent in the processes depicted in FIGS. 7, 8A, and/or 8B.

At column 7 of U.S. Pat. No. 6,750,330, it is disclosed that “The process of producing lyophilized active tubulin of the present invention can be applied to all isotypes, types, modified forms, recombinant forms, and all purity levels of tubulin since it has been performed on MAP-rich bovine brain tubulin, 70% pure and greater than 99% purified bovine brain tubulin, DEAE-Cellulose purified Hela S3 Cell line tubulin, DEAE-Cellulose purified yeast and recombinant yeast tubulin (70%-95% purity). The activity of the lyophilized tubulin product is extremely high, i.e., greater than 95% activity.” Each of these “ . . . isotypes, types, modified forms, recombinant forms, and all purity levels of tubulin . . . ” may be used as a reagent in one or more of the processes depicted in FIGS. 7, 8A, and/or 8B.

U.S. Pat. No. 6,750,330 also discloses that “Tubulin is purified by one of the methods described in Section V.(1), above, by successive cycles of polymerization and depolymerization. Active tubulin will polymerize to form microtubules which are separated from non-polymerized tubulin by differential sedimentation centrifugation which sediments the microtubules into a pellet at the bottom of the centrifuge tube. The supernatant is then removed and the tube containing the pellet is placed on ice. The pellet is then resuspended in a lyophilization buffer at concentrations between 1 μg/ml to 200 mg/mil and placed into a vessel for lyophilization.”

U.S. Pat. No. 6,750,330 also discloses that “Tubulin at 0.2 to 50 mg/ml is polymerized by incubating at a temperature from 4° C. to 45° C. for 1 to 500 minutes, and more preferably, at a temperature of 37° C. Preferably, the number of cycles of polymerization can be 1 to 6, and more preferably, about 2 to 3 for higher yields. The preferred temperature gradient for polymerization is usually less than 5 minutes from the low temperature to the high temperature. Polymerization at the increased temperature is preferably, from 1 to 500 minutes, more preferably, from 20 to 120 minutes, and most preferably, at 45 minutes. The concentration of tubulin during polymerization is preferably, between 0.2 to 50 mg/ml, more preferably, between 0.5 to 20 mg/ml, and most preferably, at 5 mg/ml.”

U.S. Pat. No. 6,750,330 also discloses that “Preferably, the temperature for centrifugation is between 15° C. to 45° C., and more preferably, the temperature is 37° C. Preferably, the sample is centrifuged at 5,000×g to 500,000×g, more preferably, 30,000×g to 200,000×g, and most preferably, at 100,000×g. The period of centrifugation is preferably, for 5 to 5000 minutes, more preferably, for 10 to 1000 minutes and, most preferably, for 30 minutes. The supernatant is removed by decanting and the tube containing the pellet is placed on ice.”

U.S. Pat. No. 6,750,330 also discloses that “Prior lyophilization methods for tubulin have been unsuccessful. The present invention produces highly active lyophilized tubulin and is applicable to a wide range of uses. This invention disclosed herein illustrates that tubulin that is lyophilized at higher concentrations is more active than tubulin lyophilized at lower concentrations. It was determined that there was a 40% loss in activity for tubulin at 3 mg/mil compared to 20 mg/ml. Thus, the concentration at which the pellet is resuspended prior to lyophilization is very critical. Preferably, pellet resuspension can be at a protein concentration of 1 μg/ml to 200 mg/ml, more preferably, between 0.5 to 50 mg/ml, and most preferably, at 20 mg/ml.”

U.S. Pat. No. 6,750,330 also discloses that “The tubulin lyophilization solution includes a buffer, a sugar, a carbohydrate polymer, a nucleotide, and a substitute protein. Buffers include, but are not limited to, PIPES, MES, Tris buffer and phosphate buffer. Preferably, the buffer is PIPES at a concentration of 15 mM, pH 6.9. Sugars include but are not limited to, sucrose, glucose, maltose and galactose. Preferably, the sugar is sucrose at 5% w/v. Carbohydrate polymers include, but are not limited to, dextran, polyethylene glycol and FICOLL. Preferably, the carboydrate polymer is FICOLL.™. (400 Kdal) at 1% w/v. Salts include, but are not limited to, MgCl2, MnCl2, CaC2, and magnesium acetate. Preferably, the salt is MgCl2, at 0.5 mM. Nucleotides include, but are not limited to, adenosine triphosphate (ATP), guanosine triphosphate (GTP), and guanosine diphosphate (GDP). Preferably, the nucleotide is GTP, at 0.5 mM. Substitute proteins include, but are not limited to, Bovine Serum Albumin (BSA) and Imunoglobulins like IgG. Preferably, the substitute protein is BSA, at 10 mg/ml. These substitute proteins can substitute for up to 50% of tubulin. Alternatively, the pellet may also be suspended in distilled water alone for a semi-stable, less than optimal formulation.” One or more of such “ . . . buffer, a sugar, a carbohydrate polymer, a nucleotide, and a substitute protein . . . ” may be used as a reagent in the process depicted in FIGS. 7, 8A, and/or 8B.

U.S. Pat. No. 6,750,330 also discloses that “The pellet is preferably air-dried or frozen; more preferably it is air-dried. Lyophilization is conducted preferably at a temperature between −200° C. to 60° C., more preferably at −45° C. to 30° C., and most preferably at −40° C. for frozen samples and 4° C. for air-dried liquid samples. The water content (v/v) in the sample is preferably between 0% to ran 20%, more preferably between 0.2% to 5%, and most preferably between 1% to 3%. Lyophilization is preferably performed at a vacuum pressure between 76 torr to 1 milli-torr, more preferably between 10 torr to 20 milli-torr, and most preferably at 100 milli-torr. V.(4) Vessels for Lyophilization.”

U.S. Pat. No. 6,750,330 also discloses that “Different applications for lyophilized tubulin require different vessels for lyophilization of tubulin. The vessels include, but are not limited to, single vials for all applications, wells in 96-well, 384-well, 864-well and higher well plates, wells and walls of the wells in 96-well, 384-well, 864-well and higher well plates, glass slides, solid supports, dip sticks, filters, frozen liquid drops, and any micro or nano-sized reaction chambers that may be available in the future. V.(5) Storage of Lyophilized Tubulin.” One or more of such “single vials” and/or “wells” may be used as storage 415 (see FIG. 8B).

U.S. Pat. No. 6,750,330 also discloses that “After lyophilization the product can be stored at −189° C. to 37° C. with desicant for greater than one year (See FIG. 3) which can be extrapolated to greater than five years at 4° C. Prior storage methods involved freezing tubulin solution in liquid nitrogen and storing at −70° C. (Shelanski et al. 1973). Storage at −70° C. is unsuitable for high through-put screening and other uses because retrieving the vials requires dexterity that cannot be automated easily. Thus, the lyophilization and subsequently, the storage methods of the present invention offer significant advantages over the methods described in the prior art. V.(6).”

Referring agan to FIG. 7, and in optional step 416, the isolated dimers are preferably disposed in separate containers. These containers may be, e.g., “single vials.

Referring again to FIG. 7, and in step 418 thereof, and in the preferred embodiment depicted therein, gamma tubulin is charged to the reaction mixture 428 to nucleate the assembly of microtubules. As is disclosed in U.S. Pat. No. 6,346,389, the entire disclosure of which is hereby incorporated by reference into this specification, “ . . . gamma.-tubulin is a phylogenetically conserved component of microtubule-organizing centers that is essential for viability and microtubule function (T. Horio et al. (1994) J. Cell Biol. 126(6): 1465-73). It is exclusively localized at the spindle poles (also known as spindle pole bodies, SPB) in mitotic animal cells, where it is required for microtubule nucleation (M. A. Martin et al. (1997) J. Cell Sci. 110(5): 623-33; I. Lajoie-Mazenc et al. (1994) J. Cell Sci. 107(10): 2825-37).gamma.-tubulin is also found on osmiophilic material that lies near the inner surface of the nuclear envelope, immediately adjacent to the SPB (R. Ding et al. (1997) Mol. Biol. Cell 8(8): 1461-79).”

The gamma-tubulin may be charged, e.g., via line 420 (see FIG. 8A), and it produces nucleating end 422 as it is allowed to polymerize in step 424 (see FIG. 8B).

In addition to charging the gamma globulin, one may charge other reagents conventionally used in tubulin polymerization. Thus, e.g., one may charge MES buffer and guanosine triphosphate via line 420.

In general, one may use reagents and conditions typically used during microtubule assembly and/or microtubule disassembly and/or microtubule stabilization, depending upon the stage of the reaction, which product(s) one wishes to produce at such stage, and which product(s), if any, one wishes to remove from the reaction mixture 428 at such stage. Thus, by way of illustration and not limitation, one may use the conditions described in Example 20 of U.S. Pat. No. 5,409,953, the entire disclosure of which is hereby incorporated by reference into this specification. As is disclosed in such Example 20, “The assembly reaction at 37° C. was followed turbidimetrically as described by Hamel et al, Biochem. Pharmacal., 32, p. 3864, 1983; and Batra et al, Molecular Pharm. 27, pp 94-102, 1984. Each 0.25 ml reaction mixture contained 1.5 mg/ml of tubulin and 0.5 mg/ml of microtubule-associated proteins (proteins were purified as described by Hamel et al, Biochemistry, 23, p. 4173, 1984, 0.1M 4-morpholine ethanesulfonate (adjusted to pH 6.6 with NaOH), 0.5 mM MgCl2 0.5 mM guanosine 5′-triphosphate, and drugs as required. The concentration of drug needed to inhibit the extent of assembly by 50% was determined.” Thus, e.g., one may use either such microtubule associated protein(s) and/or such tubulin assembly inhibitor as a reagent to be added to reaction mixture 428.

By way of further illustration, one may use the conditions described in Example 4 of U.S. Pat. No. 5,760,092, the entire disclosure of which is hereby incorporated by reference into this specification. As is disclosed in such Example 4, “Tubulin was prepared from fresh calf brains (one hour maximum after slaughter) by a modified Weisenberg procedure (Weisenberg et al, Biochemistry, 7:4466 (1968)); Na and Timasheff, Biochemistry, 19:1347 (1980). Protein aliquots (40 mg, 40-50 mg/ml) were stored in liquid nitrogen in a buffer that consisted of 0.01M sodium phosphate, 0.1 mM GTP, 0.5 mM MgCl2, 1M sucrose, pH 7.0. Prior to each assembly experiment, samples of tubulin were thawed at 20° C. and the bulk of the sucrose was removed from the tubulin solution by a Sephadex G-25 dry column procedure (Na and Timasheff, 1980). The resulting protein solution was cleared of aggregates by centrifugation at 35,000 g for 30 minutes. The final equilibration of the protein with the assembly buffer was by gel chromatography on a Sephadex G-25 column (Na and Timasheff, Methods Enzymol., 85:393 (1982)). The protein was maintained on ice and used within 4 hours of sucrose removal. Tubulin concentrations were determined spectrophotometrically at 275 nm in 6M guanidine hydrochloride (Na and Timasheff, J. Mol. Biol., 15:165 (1981)).” Thus, e.g., one may use GTP and/or one or more salts of magnesium as a reagent to be added to the reaction mixture 428.

U.S. Pat. No. 5,760,092 also discloses that “The self-assembly of tubulin was monitored turbidimetrically (Gaskin et al., J. Mol. Biol., 89:737 (1974); Lee and Timasheff, Biochemistry, 16:1754 (1977)) at 350 nm on a Cary 118 recording spectrophotometer. It is known that the turbidity is proportional to the mass of microtubules formed. For the inhibition studies, tubulin, equilibrated with assembly buffer (0.01M sodium phosphate, 16 mM MgCl2, 3.4M glycerol, 1 mM GTP, pH 7.0), was supplemented with increasing concentrations of the drug (7-acetamido-allocolchinone in this case) by addition of a concentrated stock solution of the drug in DMSO. The concentration of 7-acetamido-allocolchinone was determined by absorbance at 300 nm using 15,540 M−1 cm31 l as the extinction coefficient. The final concentration of DMSO never exceeded 1%. The solution was then incubated at 20° C. for 30 minutes prior to assembly. The protein solution was then transferred into a thermostatted cuvette maintained at 10° C. and assembly was initiated by rapidly switching the water supply to a second water bath maintained at 37° C. The development of turbidity was monitored and recorded in the spectrophotometer chart recorder. The results are summarized in FIG. 1, which shows the decrease in plateau turbidity induced by the addition of increasing amounts of 7-acetamido-allocolchinone. Tubulin concentration was 2.1×10-5 M; the concentration of 7-acetamido-allocolchinone was (a) 0.0M, (b) 1.1×10-7 M, (c) 1.9×10⁻⁷ M and (d) 5.04×10-7 M.” Thus, e.g., one may use such “assembly buffer” as one or more of the reagents to be charged to the reaction mixture 428.

By way of yet further illustration, one may use the process described in Example 4 of U.S. Pat. No. 6,140,362, the entire disclosure of which is hereby incorporated by reference into this specification. As is disclosed in such Example 4, “Compounds were evaluated for their ability to inhibit tubulin assembly into microtubules by comparing the extent of cold-reversible assembly in the presence of each test compound with controls lacking the test compound. Tubulin was isolated from calf brain tissue by two cycles of assembly/disassembly as described by Vallee, R. B. in Methods in Enzymology, vol. 134, pp. 89-104. Assay mixtures contained 1 mg/ml of purified tubulin in 1 M sodium glutamate, pH 6.6, 1 millimolar (mM) MgCl2, and the test compound, which was added as a solution in DMSO. The final concentration of DMSO in each assay was 2% (v/v). Assay mixtures were preincubated at 37° C. for 1 h, then chilled on ice for 5 min. Microtubule assembly was initiated by addition of guanosine triphosphate (0.1 mM), and incubation at 37° C. Assembly was followed turbidimetrically at 350 nm for 20 minutes (min) using a temperature-controlled cell in a Cary 2200 spectrophotometer. Since microtubules undergo depolymerization at 0° C., assembly was confirmed by measuring the reduction in turbidity following incubation for 30 min at 0° C. The difference in absorbance before and after incubation for 30 min at 0° C. (.DELTA.A350) represents the extent of microtubule assembly. Inhibition of assembly was calculated by subtracting the (.DELTA.A350) values for treatments with test compounds from the (.DELTA.A350) for controls without test compound, and expressing this difference as a percentage of the (.DELTA.A350) value for the control. Results including test compound number, test compound concentration in micromoles per liter (μM) and percent inhibition are set forth in Table 11.”

Referring again to FIG. 7 (step 424), the gamma tubulin is allowed to polymerize to a specified degree. In one embodiment, the gamma tubulin is allowed to polymerize until at least about 90 percent of the gamma tubulin monomer is in polymeric form. The extent to which the gamma tubulin has been polymerized my be determined by conventional means such as, e.g., a turbidity meter 426 that is adapted to measure the optical density of the reaction mixture 28 by “turbidimetry,” which is “The quantititative determination of a substance in suspension that is based on measurements of the decrease in light transmission by the suspension due to the scattering of the light by the suspended particles.” Reference may be had, e.g., to page 497 of the aforementioned Stensch dictionary. Referenced also may be had, e.g., to U.S. Pat. No. 4,006,988 (photo-electric depth or turbidity meter for fluid suspensions), U.S. Pat. No. 4,263,511 (turbidity meter), U.S. Pat. No. 4,863,690 (measuring instrument for bioluminescence and chemiluminescence or turbidimetry) U.S. Pat. No. 4,999,514 (turbidity meter with parameter selection and weighting), and the like. The entire disclosure of this United States patent application is hereby incorporated by reference into this specification.

In one embodiment, as different turbidimetry measurements are made by turbidity meter 426, different products are removed via line 405 to be separated (in separator 403) and/or analyzed (in analyzer 401) and stored (in storage 415) and/recycled into reaction mixture 428 via line 407. Thus, e.g., in step 430 (see FIG. 7), unreacted gamma tubulin may be removed from the reaction mixture 428 via line 405. Alternatively, or additionally, partially reacted gamma tubulin may be removed from the reaction mixture 428 via line 405.

Some or all of the unreacted tubulin (and/or some or all of the partially reacted tubulin) may be removed from the reaction mixture 428 via line 405 and analyzed and/or separated by conventional means such as, e.g., size exclusion column chomratography. Reference may be had, e.g., to U.S. Pat. No. 4,687,814 (crosslinked copolymers and their application to size exclusion chromatography), U.S. Pat. No. 4,762,617 (size-exclusion chromatography system for macromolecular interaction analysis), U.S. Pat. No. 5,190,658 (method for size exclusion chromatography), and the like. The entire disclosure of each of these United States patents is hereby incorporated by reference into this specification.

Referring again to FIG. 7, and in step 434 thereof, a tubulin dimer 435 (depicted by a circle with a plus sign within it in FIG. 8) is charged to the reaction mixture. In the embodiment depicted, tubulin dimer 435 has a positive polarity.

The tubulin dimer 435 with a positive polarity may be made from one or more of the alpha- and beta-tubulins which were characterized and isolated in steps 412 and 414. Alternatively, one may prepare one or more tubulin monomers or dimers with the required polarity and charge distribution by conventional means.

Thus, by way of illustration, one may determine the amino acid sequences of tubulin monomers by reference, e.g., to standard tubulin databases. Thereafter, one may make appropriate substitutions of amino acids to change the charge polarity and/or the charge density of the tubulin monomer, and one may then synthesize the modified monomer by conventional polypeptide synthesis techniques and apparatuses. Reference may be had, e.g., to U.S. Pat. No. 3,948,821 (solid amino acid products for polypeptide synthesis), U.S. Pat. No. 4,192,798 (rapid, large scale, automatable high pressure peptide synthesis), U.S. Pat. No. 4,507,230 (peptide synthesis reagents and method of use), U.S. Pat. No. 4,581,167 (peptide synthesis and amino acid blocking agents), U.S. Pat. No. 4,599,198 (intermediates in polypeptide synthesis), U.S. Pat. No. 4,668,476 (automated polypeptide synthesis apparatus), U.S. Pat. No. 4,816,513 (automated polypeptide synthesis process), U.S. Pat. No. 4,879,371 (solid phase peptide synthesis), U.S. Pat. No. 4,950,418 (reagent for removing protective groups in peptide synthesis), U.S. Pat. No. 4,965,343 (method of peptide synthesis), U.S. Pat. No. 5,186,898 (automated polypeptide synthesis apparatus), U.S. Pat. No. 5,221,754 (reagents for rapid peptide synthesis), U.S. Pat. No. 5,243,038 (construction of synthetic DNA and its use in large polypeptide sequences), U.S. Pat. No. 5,268,423 (peptide synthesis resins), U.S. Pat. No. 5,286,789 (solid phase multiple peptide synthesis), U.S. Pat. No. 5,373,053 (peptide synthesis method and solid support for use in the method), U.S. Pat. No. 5,567,797 (kits for protein synthesis), U.S. Pat. No. 5,591,646 (method and apparatus for peptide synthesis and screening), U.S. Pat. No. 5,637,719 (reagents for rapid peptide synthesis), U.S. Pat. No. 5,763,284 (methods for peptide synthesis and purification), U.S. Pat. No. 5,849,954 (method of peptide synthesis), U.S. Pat. No. 5,895,783 (method for in vitro protein synthesis), U.S. Pat. No. 5,942,061 (peptide synthesis with sulfonyl protecting groups), U.S. Pat. No. 6,015,881 (methods and compositions for peptide synthesis), U.S. Pat. No. 6,028,172 (reactor and method for solid peptide synthesis), U.S. Pat. No. 6,103,489 (cell-free protein synthesis system), U.S. Pat. No. 6,143,517 (thermostable proteolytic enzymes and uses thereof in peptide and protein synthesis), U.S. Pat. No. 6,204,361 (method of peptide synthesis), U.S. Pat. No. 6,320,025 (solid phase peptide synthesis reaction vessel), U.S. Pat. No. 6,680,365 (methods and compositons for controlled polypeptide sequences), U.S. Pat. No. 6,632,922 (methods and compositions for controlled polypeptide synthesis), U.S. Pat. No. 6,686,446 (methods and compositions for controlled polypeptide synthesis), U.S. Pat. No. 6,767,993 (methods and compositions for peptide synthesis), and the like. The disclosure of each of these United States patents is hereby incorporated by reference into this specification.

As will be apparent to those skilled in the art, one may charge via line 436 a modified tubulin 435 with a positive charge polarity that has a specified charge distribution dictated by the choice of amino acids one wishes to include in the tubulin monomers used in the tubulin dimer. Alternatively, or additionally, one may charge via line 442 a modified tubulin 437 with a negative charge polarity that has a specified charge distribution dictated by the choice of amino acids one wishes to include in the tubulin monomers used in the tubulin dimer.

In one embodiment, instead of synthesizing the desired tublin monomers and dimers, one may prepare the products with the desired amino acid sequence(s) by modifying a DNA sequence so that it expresses the desired sequence of amino acids in a living system. As is known to the art, one may construct desired DNA sequences with polymerase chain reaction (PCR) assemblers. As is disclosed at page 373 of the aforementioned Stensch dictionary, the polymerase chain reaction is “A technique for the synthesis of large quantities of specific DNA segments; consist of a series of repetitive cycles, one step of which involves a high temperature. The latter inactivates the DNA polymerase originally used, thus requiring the addition of fresh enzyme at each cycle.” Referene also may be had to, e.g., U.S. Pat. No. 6,143,496 (method of sampling, amplifying, and quantifying segment of nucleic acid, polymerease chain reaction assembly having nanoliter-sized sample chambers), U.S. Pat. No. 6,391,559 (method of sampling, amplifying, and quantifying segment of nucleic acid, polymerease chain reaction assembly having nanoliter-sized sample chambers), and the like. The entire disclosure of each of these United States patents is hereby incorporated by reference into this specification.

Referring again to FIG. 7, and also to FIG. 8, the process steps 412, 414, 416, 418, 424, 430, 438, and/or 438 may be repeated in whole or in part to produce a variety of tubulin assemblies with different properties. At different stages of the reaction, different tubulin assemblies, or mixtures thereof, may be removed from the reaction mixture 405 and/or stabilized and/or modified. A description of some of the “reagents” that may be added to the reaction mixture 428 and/or the analyzer 401 and/or the separator 403 is described in the next section of this specification.

Reagents that Interact with Tubulin, Tubulin Dimers, or Microtubules

As indicated in the prior section of this specification, there are a variety of different materials that interact with tubulin monomers and/or tubulin dimers and/or tubulin reaction products (such as microtubules); these may be added in the processes described in FIGS. 7, 8A, and 8B to produce a wide variety of tubulin products with different charges and/or different physical properties.

One may add to the reaction mixture 428 certain agents that either foster or inhibit the assembly of tubulin to form microtubules. Many of these agents selectively foster or inhibit the assembly of certain tubulins and not others. Thus, by the use of certain selective agents, one may drive the reaction(s) that occur in one direction but another.

As is disclosed in U.S. Pat. No. 4,904,697, the entire disclosure of which is hereby incorporated by reference into this specification, one may inhibit the polymerization of tubulin to form microtubules with the use of certain chalcone derivatives, or with colchicines. One or more of thiese chalcone derivatives, or one or more colchicines, may be added to the reaction mixture 428 whenever it is desired to selectively inhibit the tubulin polymerization.

As is disclosed in the abstract of U.S. Pat. No. 4,996,237, the entire disclosure of which is hereby incorporated by reference into this specification, “The African tree Combretum caffrum (Combretaceae) has been found to contain an agent which is a powerful inhibitor of tubulin polymerization (IC50 2-3 μM), the growth of murine lymphocytic leukemia (L1210 and P388 with ED50<0.003 mg/ml and human colon cancer cell lines (e.g. VoLo with ED50<0.01 μg/ml). This agent is herein denominated “combretastatin A-4”. The structure assigned by spectral techniques was confirmed by synthesis. “Such “combre3statin A-4” may be added to the rection mixture 428 whenever it is desired to selectively inhibit tubulin polymerization.

The reversible nature of tublin assembly is discussed in U.S. Pat. No. 5,189,055, the entire disclosure of which is hereby incorporated by reference into this specification. As is disclosed in column 1 of such patent, “Tubulin is a cell protein with a molecular weight of the order of 110,000 to 120,000 daltons, consisting of two closely associated subunits, alpha. and β. It constitutes a basic component whose assembly in helicoid form permits the construction of complex macromolecular structures commonly known as microtubules. The latter are encountered in practically all eukaryotic cells and are used in the formation of many cytoplasmic structures: mitotic spindle, centrioles, flagellae, axonemes, neurotubules, etc. Microtubules thus have fundamental roles, not yet all enumerated, in the life of the cell (division, motility, transport, growth, etc.). The assembling of tubulin is a reversible dynamic mechanism subject to a regulation which has not at present been elucidated.”

As is also disclosed in U.S. Pat. No. 5,189,055, “After extraction of the protein (from pig brain), it is possible to monitor in vitro its assembling and dismantling behaviour under the effect of varying different physicochemical parameters: polymerization in the form of microtubules following a temperature rise to 37° C.; promoted by the presence of GTP, polycations, glycerol, etc.,—depolymerization caused by a low temperature (4° C.) and promoted by Ca2+ ions, excess GTP, etc.” One may use such calcium ions and/or excess GTP or cold to, e.g., selectively depolymerize the microtubules formed in the reaction mixture 428 during part or all of the reaction process.

U.S. Pat. No. 5,189,055 also discloses that “A number of natural substances are capable of binding to specific tubulin receptor sites. They inhibit its polymerization (colchicine, vinblastine, vincristine, podophyllotoxin, etc.) or its depolymerization (taxol, rhazinilam) and can cause its spiralization (vinblastine). The present invention has been directed towards substances exhibiting biaryl character, capable of interacting with tubulin and hence exhibiting activity as a mitotic spindle poison. To this end, compounds containing a phenylpyrrole skeleton have been synthesized.” One or more of these “phyenylpyrrole skeleton compounds” may selectively be added to the reaction mixture 428 whenever it is desired to, at specified times, to inhibit tubulin polymerization or depolymerization.

As is disclosed in U.S. Pat. No. 5,760,092, the entire disclosure of which is hereby incorporated by reference in to this specification, one may add to the reaction mixture 428 an inhibitor of microtubule assembly. This patent claims (in claim 1) “1. An inhibitor of microtubule assembly comprising an allocolchinone.” Such inhibitor may be added at any stage of the reaction process, or to any particular mixture of reagents and/or reaction products, to obtain the desired results.

The inhibition mechanism of allocolchinone is discussed in U.S. Pat. No. 5,760,092, wherein it is disclosed that “Colchicine . . . is an alkaloid having a tricyclic ring structure . . . . Colchicine binds to the protein tubulin irreversibly. Tubulin is part of the cellular cytoskeleton, of the mitotic apparatus, of neurons and a building block of microtubules. The binding of colchicine to tubulin interferes with microtubule-dependent cell processes. One important example of a microtubule-dependent process with which colchicine interferes is the assembly of microtubules during metaphase. Inhibition of microtubule assembly results in the inability of a cell to move its chromosomes during cell division causing the cell to arrest during metaphase and die. Consequently, colchicine acts as an anti-mitotic agent.” One may add such colchicines to the reaction mixture 428 during part or all of the reaction process to selectively inhibit tubulin assembly.

U.S. Pat. No. 5,760,092 also discloses that “Many anti-cancer drugs act by causing cell death during mitosis. However, the use of colchicine as an anti-cancer drug is precluded by its high toxicity. The toxicity of colchicine is thought to be due in part to the fact that colchicine binds irreversibly to tubulin. Consequently, the treatment of cancer could be greatly advanced with new drugs that inhibit microtubule assembly by binding to tubulin but which are less toxic than colchicine.”

U.S. Pat. No. 5,760,092 also discloses that “Colchicine has long been used as an agent against inflammatory disease, such as gout and Mediterranean fever. Colchicine is also used to treat other diseases, such as multiple sclerosis, primary biliary cirrhosis, Alzheimer's Disease and Behcet's Disease. Thus, there is also a need for less toxic drugs having greater effectiveness with reduced side effects against these diseases.”

U.S. Pat. No. 5,760,092 also discloses that “The present invention is based on the discovery that allocolchinones, like colchicine, bind to tubulin. However, the binding of allocolchinones to tubulin is reversible, in contrast to colchicine. Certain allocolchinones are also more effective than colchicine in inhibiting microtubule formation in vitro. In addition, it has been found that the concentration of 7-acetamido-allocolchinone at which 50% of the cell growth is inhibited is about 100 fold lower than colchicine against most of the tumor cell lines in the National Cancer Institute's (NCI) revised anti-cancer screen (Grever et al., Seminars in Oncology 19:622 (1992), Alley et al., Cancer Research 48:589 (1988) and Montes et al., J. National Cancer Institute 83:757 (1991)). 7-Butyramido-allocolchinone is also active in the NCI screen” One may use such allocolchinones, and may adjust the reaction conditions appropriately, to selectively inhibit tubulin assembly.

U.S. Pat. No. 5,886,025, the entire disclosure of which is hereby incorporated by reference into this specification, discloses that certain methoxy- and ethoxy-substituted thiophenes inhibit tubulin polymerization. A discussion of “prior art” tubulin polymerization inhbhitors is presented at column 1 of the patent, wherein it is disclosed that “Antineoplastic chemotherapy currently encompasses several groups of drugs including alkylating agents, purine antagonists and antitumor antibiotics. Alkylating agents alkylate cell proteins and nucleic acids preventing cell replication, disrupting cellular metabolism and eventually leading to cell death. Typical alkylating agents are nitrogen mustard, cyclophosphamide and chlorambucil. Toxicities associated with alkylating agents treatment include nausea, vomiting, alopecia, hemorrhagic cystitis, pulmonary fibrosis and an increased risk of developing acute leukemia.”

U.S. Pat. No. 5,886,025 also discloses that “Purine, pyrimidine and folate antagonists are cell cycle and phase specific and, in order to promote an anti-tumor effect, they require cells to be in the cell replication cycle and in the DNA synthesis phase of replication. The purine antagonists such as 6-mercaptopurine or 6-thioguanidine inhibit de novo purine synthesis and interconversion of purines. The pyrimidine antagonists, such as cytarabine, 5-fluorouracil or floxuridine, inhibit DNA synthesis by inhibiting deoxycytidylate kinase and DNA polymerase.

As is also disclosed in U.S. Pat. No. 5,886,025, “Folate antagonists, e.g., methotrexates, bind tightly with the intracellular enzyme dihydrofolate reductase ultimately leading to cell death resulting from an inability to synthesize pyrimidines. Toxicities associated with the use of these compounds include alopecia, myelosuppression, vomiting, nausea, and cerebellar ataxia, among others.”

U.S. Pat. No. 5,886,025 also discloses that “Plant alkaloids such as vincristine, vinblastine or podophyllotoxins etoposide and teniposide generally inhibit mitosis and DNA synthesis and RNA dependent protein synthesis. Toxicities of these drugs are similar to those described above and include myopathy, myelosuppression, peripheral neuropathy, vomiting, nausea and alopecia.” One may use one or more of thse plant alkaloids, during part or all of the reaction process, to selectively inhibit tubulin assembly.

U.S. Pat. No. 5,886,025 also discloses that “Antitumor antibiotics such as doxorubicin, daunorubicin and actinomycin act as intercalators of DNA, preventing cell replication, inhibiting synthesis of DNA-dependent RNA and inhibiting DNA polymerase. Bleomycin causes scission of DNA and mitomycin acts as inhibitor of DNA synthesis by bifunctional alkylation. Toxicities of these antibiotics are numerous and severe and include necrosis, myelosuppression, anaphylactic reactions, anorexia, dose-dependent cardiotoxicity and pulmonary fibrosis.”

U.S. Pat. No. 5,886,025 also discloses that “Other compounds used for chemotherapeutical treatment of cancer are inorganic ions such as cisplatin, biologic response modifiers such as interferon, enzymes and hormones. All these compounds, similarly to those mentioned above, are accompanied by toxic adverse reactions.” These “other compounds” may also be selectively used in the reaction mixture 428.

U.S. Pat. No. 6,107,958 discloses, at columns 40-42 thereof, an optical assay for the polymerization of microtubules; the entire disclosure of this United States patent application is hereby incorporated by reference into this specification. As is disclosed in such columns 40-42, “In the following experiments the hormonally inactive thyroid hormone analog, DIME, at 1 to 5 μM concentrations inhibits the GTP-dependent polymerization of MTP as determined by an optical test. This inhibition is critically dependent on the concentration of GTP. The quantitative correlation between the concentrations of DIME and GTP, under conditions of a linear rate of MTP polymerization, follows Michaelis-Menten kinetics and the inhibition portrays a “mixed” type, where km for GTP and Umax are altered simultaneously. Chemical analogues of DIME inhibit MTP polymerization parallel to their antitumorigenic action in vivo. The MTP site is one of the early cellular response sites of DIME.”

As is also disclosed in U.S. Pat. No. 6,017,958, “Exposure of human mammary cancer cells (MDA-MB-231) to 1 μM DIME induced abnormal spindle structures within 18 hours of drug treatment, thus a putative DIME-microtubule-protein (MTP) interaction appears to be a component of early cellular responses to the drug, Zhen, et al., 1997, “Cellular Analysis of the mode of action of methyl-3-5-diiodo-4-(4′-methoxyphenoxy) benzoate (DIME) on tumor cells”, Intl. J. Oncol. Abnormal spindle structures could be the result of DIME-MTP interaction or reactions of DIME with components of the microtubule organizing center or with as yet undefined systems sequentially or in concert. Since time-dependent quantitative analysis of the MTP system in situ is unsuitable for initial velocity measurement we adapted the in vitro assembly system of neurotubules as a model for a quantitative analysis of the interaction of DIME with MTP. As demonstrated by, Gaskin, et al., 1974, “Turbidimetric studies of the in vitro assembly and disassembly of porcine neurotubules”, J. Mol. Biol. 89:737-758; and Kirschner, et al., 1974, “Microtubules from mammalian brain: some properties of their depolymerization products and a proposed mechanism of assembly and disassembly”, Proc. Natl. Acad. Sci. U.S.A. 71:1159-1163; this system is suitable for kinetic assay of MTP assembly in vitro. The time course of MTP assembly consists of initiation and propagation and termination steps, Gaskin, et al., 1974, “Turbidimetric studies of the in vitro assembly and disassembly of porcine neurotubules”, J. Mol. Biol. 89:737-758. The rate of propagation under defined conditions is sufficiently linear to permit kinetic analysis, that can be evaluated with respect DIME and GTP concentrations. As we show here the inhibition of MTP assembly by DIME occurs in the same range of drug concentration as required to inhibit tumorigenesis in vivo, or to inhibit cell replication or induce eventual cell death; Mendeleyev, et al., 1997. “Structural specificity and tumoricidal action of methyl-3,5-diiodo-4-(4′-methoxyphenoxy) benzoate (DIME)” Int. J. Oncol., 10:689-695 and Table 8, above; therefore the DIME-MTP interaction is most probably a component of the apparently pleiotropic cellular mechanism of action of DIME.”

As is also disclosed in U.S. Pat. No. 6,017,958, “Inhibition of MTP polymerization may have highly complex cellular consequences. In cytokinesis this inhibition may interfere with traction forces of tubulin and prevent the formation of a cleavage furrow which is essential for cell division, Burton, et al., 1997, “Traction forces of cytokinesis measured with optically modified elastic substrate”, Nature 385:450-454. The inhibition of MTP *—polymerization by DIME should be correlated with the biochemical sites of this drug. As compared with Mendeleyev et al.; supra, DIME directly activates pp2-ase, therefore it is necessary to coordinate this effect with mitosis-related phenomena induced by DIME. For example it was recently reported, Kawabe, et al., 1997, “HOXII interacts with protein phosphatase pp2a and pp1 and disrupts G2/M cell cycle check point” Nature 385:454-458. that pp2-ase may regulate G2/M transition and pp2-ase is also a potential oncogene, the inhibition of which promotes oncogenesis. It is possible that activation of pp2-ase by DIME be antagonistic to oncogenesis.”

As is also disclosed in U.S. Pat. No. 6,017,958, “On the basis of these experiments, it can be seen that thyroxine type analogues, such as DIME, are capable of blocking mitosis in cancer cells. The present invention provides for a rapid screen for such compounds by use of these techniques and use of cell sorters, chromosome blot or other analysis of DNA in cells.” One may selectively use one or more of such thyroxine type analogs, during part or all of the reaction process, to selectively inhibit tubulin assembly.

By way of further illustration, U.S. Pat. No. 6,326,402 discloses that a diiodo thyronine analog binds a microtubule. The entire disclosure of this United States patent is hereby incorporated by reference into this specification. One may use such as diiodo thyronine analog as a reagent in the processes depicted in FIGS. 7, 8A, and/or 8B.

An experiment designed to determine the effect of discodermolide (and its analogs) upon tubulin polymerization was disclosed at columns 20-21 of U.S. Pat. No. 6,495,594, the entire disclosure of which is hereby incorporated by reference into this specification. As is disclosed in this patent, “Polymerization of purified bovine brain tubulin (Cytoskeleton Inc., Denver, Colo.) was followed by changes in the optical density of tubulin solutions at 350 nm in a Hitachi U-3010 spectrophotometer equipped with a SPR-10 electronic thermostatted cell holder. Stock solutions of tubulin were diluted on ice in G-PEM buffer (1 mM GTP, 80 mM PIPES, 1 mM EGTA, 0.5 mM magnesium chloride; pH 6.8) to a final concentration of 1 mg/mL. The instrument was zeroed on this solution at 4° C. Discodermolide, and its analogs, were then added to the tubulin solution to a final concentration of 10 μM, quickly mixed, and the absorbance monitored over a period of 61 minutes. Within this time the temperature of the thermoelectric cell holder was held at 4° C. for 1 minute, increased to 35° C. at a rate of 1° C./minute, reduced back to 4° C. at a rate of 2° C./minute, and held at 4° C. for an additional 14 minutes.”

U.S. Pat. No. 6,495,584 also discloses that “Cell cycle studies were initiated in order to pinpoint a specific phase within the cell cycle in which discodermolide analogs were exerting their antiproliferative effect. A549 human lung cells were used as cell cycle targets to compare the effects of discodermolide and discodermolide analogs on perturbation of the cell cycle. Cell cycle analyses were performed as follows: A549 cells were incubated at 37° C. in 5% CO₂ in air in the presence or absence of varying concentrations of discodermolide or discodermolide analogs for 24 hr. Cells were harvested, fixed in ethanol, washed, and stained with 0.02 mg/mL of propidium iodide (P.I.) together with 0.1 mg/mL of RNAse A. Stained preparations were analyzed on a Coulter EPICS ELITE flow cytometer with 488 nM excitation. Fluorescence measurements and resulting DNA histograms were collected from at least 10,000 P.I. stained cells at an emission wavelength of 690 nM. Raw histogram data was further analyzed using a cell cycle analysis program (Multicycle, Phoenix Flow Systems).”

By way of yet further illustration, and as is disclosed in U.S. Pat. No. 6,443,187 (the entire disclosure of which is hereby incorporated by reference into this specification), one may add to the reaction mixture derivatives of known tubulin-binding compounds in which a (poly) fluorobenzene, a fluoropyridine, or a fluoronitrobenzene moiety is incorporated or added to the structure. These tubulin binding agents are described at columns 3-4 of U.S. Pat. No. 6,433,187, wherein it is disclosed that “The present invention provides a variety of agents capable of covalent attachment to tubulin. Accordingly, the compounds are particularly useful as antimitotic agents for the treatment of cancer. The compounds are derivatives of naturally-occurring antimitotic agents as well as other tubulin-interacting compounds. Briefly, the compounds can be described as antimitotic agents having, for example, a pentafluorophenyl-sulfonamide group (C6 F5—SO₂—NH—), a 2-fluoropyridyl group, a nitrofluorophenyl group or a dinitrofluorophenyl group. In each instance, the reactive fluorinated aromatic moiety is introduced into the parent compound by replacing an existing portion of the parent (e.g., an aromatic ring or lactone), by attaching to an available reactive functional group (e.g., hydroxyl, amino, carboxylic acid and the like), or by attaching to an otherwise unfunctionalized portion of the molecule. Each of the reactive fluorinated aromatic moieties is capable of covalently modifying a cysteine thiol owing to the electrophilic nature of the fluoroaryl moiety and the leaving group character of the fluorine atom.”

U.S. Pat. No. 6,433,187 also discloses that “Derivatives of parent tubulin-interacting compounds are also described in which small portions of the parent compound are replaced with fragments of similar size that can increase the reactivity of the aromatic electrophile. For example, an ethylene group (—CH2 CH2—) can be replaced with a sulfonamido moiety (—SO₂ NH—) in those positions wherein the reactivity of an adjacent pentafluorophenyl or tetrafluorophenyl group can be enhanced. Additionally, any of the noted fluorinated romatic electrophiles can be attached to the remainder of the molecule via a connecting element that further enhances the reactivity of the fluorinated electrophile (e.g., a sulfonyl group or a carbonyl group).”

U.S. Pat. No. 6,433,187 also discloses that “The tubulin-interacting agents on which the following embodiments are based have been described in, for example, Jordan, et al., Med. Res. Rev. 18(4):259-296 (1998), Bai, et al., J. Biol. Chem. 271(21):12639-12645 (1996), Hamel, Med. Res. Rev. 16(2):207-231 (1996), Sackett, Pharmacol. Ther. 59(2):163-228 (1993) and Luduena, et al., Pharmac. Ther. 49:133-152 (1991).” One may use these “tubulin interacting agents” in the processes described in FIGS. 7, 8A, and/or 8B.

U.S. Pat. No. 6,433,187 also discloses that “The present invention generally provides tubulin binding agents that selectively and covalently bind to tubulin. The agents are derivatives of compounds which non-covalently bind to the colchicine binding site, the vinca alkaloid binding site, or the rhizoxin/maytansine binding site of tubulin. Additionally, the derivatives are formed by the attachment of a fluorinated aromatic electrophile to the parent non-covalent compounds, or by the replacement of a portion of the parent compound with the fluorinated aromatic electrophile. As used herein, the term derivative is also meant to include those agents in which a fluorinated aromatic electrophile is attached to the parent compound via a linker, preferably a linker which increases the electrophilic character of the fluorinated aromatic electrophile. Still further, the term “derivative” is meant to include those compounds in which small portions of the parent compound are replaced with fragments of similar size that also serve to enhance the reactivity of the fluorinated aromatic electrophile.”

U.S. Pat. No. 6,433,187 also discloses that “In other preferred embodiments, the agent is a derivative of a compound selected from the group consisting of colchicine, podophyllotoxin, combretastatin, nocodazole, stegnacin, dihydroxy-pentamethoxyflananone, 2-methoxyestradiol, vinblastine, vincristine, dolastatin, curacin A, etoposide, teniposide, sanguinarine, griseofulvin, cryptophycins or chelidonine.”

Certain amide derivatives may be used as tubulin binding agents in the process of this invention; and these agents preferentially bind covalently to beta tubulin, as is disclosed in U.S. Pat. No. 6,500,405, the entire dislclosure of which is hereby incorporated by reference into this specification,” Microtubules are intracellular filamentous structures present in all eukaryotic cells. As components of different organelles such as mitotic spindles, centrioles, basal bodies, cilia, flagella, axopodia and the cytoskeleton, microtubules are involved in many cellular functions including chromosome movement during mitosis, cell motility, organelle transport, cytokinesis, cell plate formation, maintenance of cell shape and orientation of cell microfibril deposition in developing plant cell walls. The major component of microtubules is tubulin, a protein composed of two subunits called alpha and beta. An important property of tubulin in cells is the ability to undergo polymerization to form microtubules or to depolymerize under appropriate conditions. This process can also occur in vitro using isolated tubulin.”

As is also disclosed in U.S. Pat. No. 6,500,405, “Microtubules play a critical role in cell division as components of the mitotic spindle, an organelle which is involved in distributing chromosomes within the dividing cell precisely between the two daughter nuclei. Various drugs and pesticides prevent cell division by binding to tubulin or to microtubules. Anticancer drugs acting by this mechanism include the alkaloids vincristine and vinblastine, and the taxane-based compounds paclitaxel and docetaxel {see, for example, E. K. Rowinsky and R. C. Donehower, Pharmacology and Therapeutics, 52, 35-84 (1991)}. Other antitubulin compounds active against mammalian cells include benzimidazoles such as nocodazole and natural products such as colchicine, podophyllotoxin and the combretastatins. Benzimidazole compounds which bind to tubulin are also widely used anthelmintics {McKellar, Q. A. and Scott, E. W., J. Vet. Pharmacol. Ther., 13, 223-247 (1990)}. Anti-tubulin herbicides are described in “The Biochemical Mode of Action of Pesticides”, by J. R. Corbett, K. Wright and A. C. Baillie, pp. 202-223, and include dinitroanilines such as trifluralin, N-phenylcarbamates such as chlorpropham, amiprophos-methyl, and pronamide. Fungicides believed to act by binding to tubulin include zarilamide {Young, D. H. and Reitz, E. M., Proceedings of the 10th International Symposium on Systemic Fungicides and Antifungal Compounds, Reinhardsbrunn, ed by H. Lyr and C. Polter, 381-385, (1993)}, the benzimidazoles benomyl and carbendazim, and the N-phenylcarbamate diethofencarb {Davidse, L. C and Ishi, H. in “Modern Selective Fungicides”, ed. by H. Lyr, 305-322 (1995)}.”

As is also disclosed in U.S. Pat. No. 6,500,405, “Due to the success of tubulin as a biochemical target for drugs and pesticides, there is considerable interest in discovering new compounds which bind to tubulin. Various cell-free methods are available for detecting such compounds. A common method involves measuring the ability of test compounds to inhibit the polymerization of isolated tubulin into microtubules in vitro {see for example, E. Hamel, Medicinal Research Reviews, 16, 207-231 (1996)}. In a second method, interactions of test compounds with isolated tubulin can be detected in binding assays by measuring the ability of the test compound to influence binding of a second tubulin-binding ligand, used as a probe. (The term “test compound” means a compound which one wishes to evaluate, i.e. to test, for its ability to affect tubulin). Typically, the probe is radiolabeled to enable binding to be measured. A test compound which binds to tubulin may influence binding of the probe by binding to the same site on the tubulin protein as the probe, and thus reduce the amount of probe which binds. Alternatively, binding may be influenced by means of an “allosteric” interaction in which the test compound binds to a different site from that of the probe and induces a conformational change in the tubulin protein which affects the binding site of the probe. Such an allosteric interaction may either increase or decrease binding of the probe. A third approach involves measuring the effect of test compounds on tubulin-associated guanosine triphosphatase activity {Duanmu, C., Shahrik, L. K., Ho, H. H. and Hamel, E., Cancer Research, 49, 1344-1348 (1989)}.”

As is also disclosed in U.S. Pat. No. 6,500,405, “To screen large numbers of compounds by any of these methods is feasible at present only using tubulin from mammalian brain tissue, since it has not been possible to isolate sufficiently large amounts of purified tubulin from other sources. This limits the usefulness of these methods since many anti-tubulin compounds show great specificity with respect to their effects on microtubules from different sources. For example, the herbicides oryzalin and amiprophosmethyl inhibit the polymerization of plant tubulin but not brain tubulin, whereas colchicine is more than 100-fold more effective as an inhibitor of brain tubulin polymerization than of plant tubulin polymerization {Morejohn, L. C. and Fosket, D. E., ‘Tubulin from Plants, Fungi, and Protists’, in “Cell and Molecular Biology of the Cytoskeleton”, ed. by J. W. Shay, 257-329 (1986)).”

As is also disclosed in U.S. Pat. No. 6,500,405, “The present invention relates to the use of certain amide derivatives, known to inhibit the growth of eukaryotic cells, including fungal and plant cells {see, for example, U.S. Pat. Nos. 3,661,991, 4,863,940 and 5,254,584). Said amides have now been found useful as probes in binding assays to screen compounds for antitubulin activity, a use which U.S. Pat. Nos. 3,661,991, 4,863,940 and 5,254,584 neither disclose nor suggest. While radiolabeled probes such as colchicine {see for example, M. H. Zweig and C. F. Chignell, Biochemical Pharmacology, 22, 2141-2150 (1973)} and vinblastine (see for example, R. Bai et al., Journal of Biological Chemistry, 265, 17141 (1990)) have been used extensively in binding assays using isolated tubulin, these compounds bind noncovalently to tubulin.”

As is also disclosed in U.S. Pat. No. 6,500,405, “One advantage of the amide derivatives of this invention over existing antitubulin compounds in competitive binding assays results from their unique ability to bind covalently in a highly specific manner to tubulin, specifically to the beta-subunit of tubulin. (A covalent bond is a nonionic chemical bond characterized by the sharing of electrons by two atoms). In binding assays it is necessary to measure the amount of the probe which is bound to tubulin, and this generally involves separating the tubulin-bound probe from unbound probe. In the case of the amides, since binding is covalent, the tubulin-bound probe is chemically stable allowing easy separation from the unbound probe by methods such as filtration or centrifugation. This enables their use not only in assays using isolated tubulin but also in assays using whole cells, crude cell extracts, and partially purified tubulin preparations, thus obviating the need for isolated tubulin and enabling tubulin-binding assays to be carried out in many different types of cell or cell extract.”

As is also disclosed in U.S. Pat. No. 6,500,405, “One aspect of the present invention involves use of amide probes in binding assays to screen large numbers of compounds in order to identify those compounds with antitubulin activity using whole cells, cell extracts or isolated tubulin. For example, test compounds which bind to plant or fungal tubulin may be detected in assays using plant or fungal cells, thus providing a means of detecting antitubulin compounds with herbicidal or fungicidal activity. Similarly, amide probes may be used to detect compounds which bind to tubulin in mammalian cells or cell extracts, thus providing a means of detecting antitubulin compounds with anticancer activity.”

As is also disclosed in U.S. Pat. No. 6,500,405, “A second aspect of the current invention involves use of amide probes in binding assays to evaluate the sensitivity of a cell population to an antitubulin compound. For example, the current invention can be used to evaluate the sensitivity of a tumor cell population to an antitubulin drug such as paclitaxel, vincristine or vinblastine, thus providing a means of predicting drug sensitivity of a patient's tumor at the time of diagnosis or relapse using cells isolated by biopsy, and consequently guiding selection of the optimal chemotherapy regimen. Frequently, treatment of neoplasms with a particular antitubulin drug results in resistance development due to a reduced accumulation of drug in the cell. The current invention also provides a method for determining sensitivity of such resistant cells to antitubulin drugs. Various types of in vitro drug sensitivity tests have been used to select drugs more likely to be effective against tumor cells of a particular patient prior to their in vivo application {Cortazar, P. et al., Clinical Cancer Research, 3, 741-747 (1997), Arps, H. et al., Int. J. Immunotherapy, III, 229-235 (1987)}. Such assays typically involve cell culture of the isolated tumor cells or xenotransplantation using transplant-bearing mice, and require several days to multiple weeks to obtain results. In the current invention, the sensitivity of isolated tumor cells to antitubulin drugs can be determined by measuring the ability of said antitubulin drugs to influence binding of an amide probe to the cells, cell extracts or isolated tubulin. Since this method does not require culture of the isolated cells, it can provide sensitivity data within a few hours allowing drug sensitivity to be determined more rapidly.”

As is also disclosed in U.S. Pat. No. 6,500,405, “A third aspect of the present invention involves another approach to the use of amide probes in binding assays to evaluate sensitivity of eukaryotic cells to pesticides or drugs which act by binding to tubulin. Specifically, this approach is useful in resistance monitoring for antitubulin pesticides or drugs to detect cells which show altered sensitivity to said antitubulin pesticides or drugs due to modifications in tubulin. Resistance to antitubulin compounds due to modifications in tubulin have occurred in fungal pathogens {Davidse, L. C. and Ishi, H. in “Modern Selective Fungicides”, ed. by H. Lyr, 305-322 (1995)}, algae (James, S. W. et al., Journal of Cell Science, 106, 209-218 (1993)} and helminths (Beech, R. N. et al., Genetics, 138, 103-110 (1994)}. Resistant cells containing modified tubulin may show a difference in binding affinity for amides, allowing amide probes to be used in binding assays to detect such mutants. Such an assay can be carried out by comparing the rate of binding of an amide probe to cells or extracts of cells previously exposed to the antitubulin pesticide or drug with the rate of binding to untreated control cells or cell extracts.”

As is also disclosed in U.S. Pat. No. 6,500,405, “For example, benzimidazole and thiophanate fungicides such as benomyl (methyl 1-(butylcarbamoyl)benzimidazol-2-ylcarbamate), fuberidazole (2-(2′-furyl)benzimidazole), thiabendazole (2-(4-thiazolyl)benzimidazole), carbendazim (methyl benzimidazol-2-ylcarbamate), thiophanate-methyl (1,2-bis(3-methoxycarbonyl-2-thioureido)benzene, and thiophanate (1,2-bis(3-ethoxycarbonyl-2-thioureido)benzene are known in the art for use against plant pathogenic fungi. However, the use of benzimidazole and thiophanate fungicides over a period of time can result in the development of fungal strains having reduced sensitivity to these fungicides, whereby the fungicides are much less effective in controlling a particular fungal disease. Such “resistant” fungi when isolated as pure cultures typically are from 10-fold to >1,000-fold less sensitive to benzimidazoles and thiophanates than fungi from locations which have not been exposed to these fungicides. Moreover, fungi which develop reduced sensitivity to one benzimidazole or thiophanate fungicide frequently also show reduced sensitivity to other benzimidazole or thiophanate fungicides. The N-phenylcarbamate fungicide diethofencarb is used commercially to control benzimidazole-resistant fungi such as Botrytis cinerea. However, its use has led to the development of fungal strains resistant to both benzimidazoles and diethofencarb. Current methods to detect fungal strains resistant to benzimidazoles, thiophanates or diethofencarb are labor-intensive and time-consuming. Some methods involve isolation of pure test cultures followed by in vitro assays of mycelial growth using fungicide-amended agar plates, or in vivo assays involving fungicide-treated leaves. Alternatively, slide germination tests of spores may be carried out in the presence of fungicide. Fungal strains which are resistant to diethofencarb and/or benzimidazoles and thiophanates typically contain modified tubulin proteins {see for example, Koenraadt, H. et al., Phytopathology, 82, 1348-1354 (1992) and Yarden, O. and Katan, T., Phytopathology, 83, 1478-1483 (1993)}. Benzimidazole-resistant, diethofencarb-sensitive fungal strains typically show enhanced sensitivity to amide derivatives of the present invention, whereas benzimidazole-resistant, diethofencarb-resistant fungal strains typically show reduced sensitivity. While not wishing to be bound by theory, it is believed that amide probes can be used in binding assays to differentiate benzimidazole-resistant, diethofencarb-sensitive fungal strains which show enhanced ability to bind amide probes in assays using whole cells or cell extracts, or benzimidazole-resistant, diethofencarb-resistant fungal strains which show reduced ability to bind amide probes, from strains which are not resistant. Such assays may be less labor-intensive and time-consuming, and may also provide information as to whether the resistance mechanism involves a change in tubulin. Information about the mechanism of resistance may be useful in designing a resistance management strategy.”

As is also disclosed in U.S. Pat. No. 6,500,405, “A fourth aspect of the present invention involves the use of amide probes in binding assays to detect and quantitate tubulin in cells or cell extracts. Tubulin is the subject of intense research due to its success as a target for drugs and pesticides and its important cellular functions. In such studies it is often desirable to detect and quantitate tubulin in cells or cell extracts. At present this is accomplished by various immunoassays {D. Thrower et al., Methods in Cell Biology, vol. 37, pp. 129-145 (1993)}, sodium dodecyl sulfate polyacrylamide gel electrophoresis {B. M. Spiegelman et al., Cell, vol. 12, pp. 587-600 (1977)}, binding to DEAE-cellulose {J. C. Bulinski et al., Analytical Biochemistry, vol. 104, 432-439 (1980)}, or by measuring colchicine-binding activity {Wilson, L., Biochemistry, vol. 9, pp. 4999-5007 (1970)}. Amide probes offer an alternative method to detect and quantitate tubulin based on measurement of amide-binding activity. Use of amide probes obviates the need for antibodies against tubulin, provides a simpler and more rapid method than either sodium dodecyl sulfate polyacrylamide gel electrophoresis or binding to DEAE-cellulose, and is applicable to measurement of tubulin levels in a variety of cells such as plant or fungal cells which are not sensitive to colchicine.”

One may add as a reagent to the reaction mixture 428 a tubulin depolymerization agent, such as the “Spongistatin” disclosed in U.S. Pat. No. 6,512,003, the entire disclosure of which is hereby incorporated by reference in to this specification. As is disclosed at columns 1-2 if this patent, “Cellular proliferation, for example, in cancer and other cell proliferative disorders, occurs as a result of cell division, or mitosis. Microtubules play a pivotal role in mitotic spindle assembly and cell division . . . . These cytoskeletal elements are formed by the self-association of the ad tubulin heterodimers . . . . Agents which induce depolymerization of tubulin and/or inhibit the polymerization of tubulin provide a therapeutic approach to the treatment of cell proliferation disorders such as cancer.”

U.S. Pat. No. 6,512,003 also discusses the structure of the alpha/beta tubulin dimer, stating that “Recently, the structure of the .alpha.β tubulin dimer was resolved by electron crystallography of zinc-induced tubulin sheets . . . . According to the reported atomic model, each 46×40×65 Angstrom. tubulin monomer is made up of a 205 amino acid N-terminal GTP/GDP binding domain with a Rossman fold topology typical for nucleotide-binding proteins, a 180 amino acid intermediate domain comprised of a mixed B sheet and five helices which contain the taxol binding site, and a predominantly helical C-terminal domain implicated in binding of microtubule-associated protein (MAP) and motor proteins . . . .”

U.S. Pat. No. 6,512,003 then discussed certain tubulin-binding molecules, including “Spongistatin.” At column 1 of such patent it is disclosed that “Novel tubulin-binding molecules which, upon binding to tubulin, interfere with tubulin polymerization, can provide novel agents for the inhibition of cellular proliferation and treatment of cancer. Spongistatin . . . is a potent tubulin depolymerizing natural product isolated from an Eastern Indian Ocean sponge in the genus Spongia . . . . Spongistatins are 32-membered macrocyclic lactone compounds with a spongipyran ring system containing 4 pyran-type rings incorporated into two spiro[5.5]ketal moieties . . . . In cytotoxicity assays, spongistatin (SP) exhibited potent cytotoxicity with subnanomolar IC50 values against an NCI panel of 60 human cancer cell lines . . . . SP was found to inhibit the binding of vinc alkaloids (but not colchicin) to tubulin8, indicating that the binding site for this potent tubulin depolymerizing agent may also serve as a binding region for vinc alkaloids.” Such spongistatin may be used as a reactant in reaction mixture 428 to selectively bind to certain tubulins.

U.S. Pat. No. 6,512,003 also discloses that “Novel tubulin binding compounds, which, upon binding to tubulin, interfere with tubulin assembly, for example by causing depolymerization of tubulin or by inhbiting tubulin polymerization, would provide novel agents for the prevention of cellular proliferation, for example in the inhibition of tumor cell growth and treatment of cancer.” The patent goes on to disclose certain spiroketal pyranes that bind to a specified binding pocket in tubulin, stating that “A novel binding pocket has been identified in tubulin, which binding pocket accepts and binds novel, small molecule tubulin binding spiroketal pyrane compounds of the invention. Binding of the spiroketal pyranes . . . to tubulin causes tubulin depolymerization, and/or inhibits tubulin polymerization. The spiroketal pyranes of the invention are therapeutically effective as cytotoxic agents, to inhibit cellular proliferation, and as effective anti-cancer agents.” One or more of these spiroketal pyranes may be added to the reaction mixture 428 to selectively inhibit tubulin assembly.

By way of further illustration, one may add one or more sulfonylurea compounds to the reaction mixture 428 to selectively inhibit tubulin assembly; these compounds are discussed in U.S. Pat. No. 6,586,188, the entire disclosure of which is hereby incorporated by reference into this specification.

By way of further illustration, one may add to the reaction mixture 428 a microtubule associated protein such as, e.g, the “MAP4” protein disclosed in U.S. Pat. No. 5,998,148, the entire disclosure of which is hereby incorporated by referenrence into this specification. As is known to those skilled in the art, microtubule associated proteins are high molecular weight proteins, with molecular weights from about 200,000 to about 300,000, that are associated with and ehance the polymerization of microtubules.

An excellent discussion of microtubule associated proteins is presented in column 1 of U.S. Pat. No. 5,998,148, wherein it is disclosed that “In order to maintain their shape and integrity, it is critical that all types of cells contain a structural scaffold. This structure is known as the cytoskeleton and is composed of a framework of interlocking proteins such as microtubules, actin and intermediate filaments. It is currently believed that the controlled regulation of the assembly and disassembly of the cytoskeleton is critical to the survival of the cell and many cellular processes are mediated by the cytoskeleton, especially those involving the interaction of the cell with the surrounding environment. These processes include but are not limited to cell adhesion, motility, and polarity. Cell division or mitosis is also dependent on concerted structural changes in the cytoskeleton.”

U.S. Pat. No. 5,998,148 also discloses that “There are several proteins that, in conjunction with the primary components of the cytoskeleton, act as regulators of cytoskeletal architecture. Microtubule-associated proteins (MAPs) comprise one group of proteins that mediate microtubule assembly and function required for the maintenance of cytoskeletal //integrity. MAPs co-purify with microtubule polymers and are defined by their association with the microtubule lattice. These proteins are divided into two classes; motor MAPs which play an integral part in cellular movement, and structural MAPs which dictate the morphologic characteristics of the cell (Maccioni and Cambiazo, Physiol. Rev., 1995, 75, 835-864; Olmsted, Annu. Rev. Cell Biol., 1986, 2, 421-457).”

U.S. Pat. No. 5,998,148 also discloses that “Microtubule-associated protein 4 (also known as MAP4) is a member of the non-neuronal structural MAP family. Studies comparing the bovine, human, and mouse MAP4 sequences demonstrated an 80% similarity among the proteins indicating that they belong to the same family of MAPs (West et al., J. Biol. Chem., 1991, 266, 21886-21896).”

U.S. Pat. No. 5,998,148 also discloses that “Originally isolated from microtubule preparations of differentiated mouse neuroblastoma cells, MAP4 was shown to be encoded by a single gene that expresses multiple transcripts in a tissue-specific manner (Code and Olmsted, Gene, 1992, 122, 367-370; Parysek et al., J. Cell Biol., 1984, 99, 2287-2296). These studies implicate MAP4 in the mediation of processes common to supportive and connective tissue types in the mouse. Further support of this conclusion comes from studies in which a muscle-specific MAP4 transcript was isolated in the mouse and shown to be required for myogenesis (Mangan and Olmsted, Development, 1996, 122, 771-781). In these studies, a plasmid bearing the muscle MAP4 nucleotides 216-1214 in the reverse orientation was transfected into myoblasts.”

U.S. Pat. No. 5,998,148 also discloses that “MAP4 is believed to affect microtubule dynamics by stabilizing the microtubule lattice (Illenberger et al., J. Biol. Chem., 1996, 271, 10834-10843). It has been shown that this stability is disrupted upon phosphorylation and recently at least two kinases have been reported that phosphorylate MAP4, cdc2 kinase which phosphorylates MAP4 in the M (mitosis) phase of the cell cycle and p110mark kinase (Illenberger et al., J. Biol. Chem., 1996, 271, 10834-10843; Ookata et al., Biochemistry, 1997, 36, 15873-15883).”

U.S. Pat. No. 5,998,148 also discloses that “Overexpression of the full- or partial-length (containing only the microtubule binding domain) MAP4 protein was shown to retard cell growth and inhibit organelle motility and trafficking in vivo (Bulinski et al., J. Cell Sci., 1997, 110, 3055-3064; Nguyen et al., J. Cell Sci., 1997, 110, 281-294). MAP4 expression has been shown to be elevated in cells with mutant p53 oncogene expression and therefore linked to cancer chemotherapeutic drug sensitivity. Immunofluorescent studies of murine fibroblasts transfected with MAP4 revealed that cells overexpressing MAP4 were more sensitive to the cancer drug paclitaxel, and less sensitive to vinca alkaloid treatment (Zhang et al., Oncogene, 1998, 16, 1617-1624).”

One may add to the reaction mixture 428, during a portion of or all of the reaction process, or to some or all of the reaction mixture, a microtubule stabilizing agent such as, e.g., the stabilizing agents disclosed in U.S. Pat. No. 5,616,608, the entire disclosure of which is hereby incorporated by reference into this specification. Claim 1 of this patent describes “1. A method of preventing or reducing a fibroproliferative vascular disease in a patient comprising: treating said patient with a pharmaceutical preparation comprising a therapeutically effective amount of a microtubule stabilizing agent selected from the group consisting of taxol, a water soluble taxol derivative, and deuterium oxide.” Similarly, one may use one or more of the microtubules stabilizing agents discussed in U.S. Pat. No. 6,403,635 (taxol or taxol derivative stabilizer), U.S. Pat. No. 6,414,015 (laulimalide microtubule stabilizing agent), U.S. Pat. No. 6,429,232 (taxol), U.S. Pat. No. 6,495,594 (biologically active analogs of doscodermolide), U.S. Pat. No. 6,660,767 (coumarin), U.S. Pat. No. 6,719,540 (C3-cyano epothione derivatives), U.S. Pat. Nos. 6,740,751, 6,677,370 (dictyostatin compounds), and the like. The entire disclosure of each of these United States patents is hereby incorporated by reference into this specification.

One may add during part or all of the reaction processs, to part or all of the reaction mixture, an anti-microtubules agent such as, e.g, the anti-microtubule agent disclosed in U.S. Pat. Nos. 6,633,347, 6,593,321, and 6,515,016, the entire disclosure of each of which is hereby incorporated by reference into this specification. Claim 1 of this patent describes “1. A method for treating or preventing disease of the pericardium, heart, or coronary vasculature, comprising administering intrapericardially to a patient an anti-microtubule agent, such that said disease of the pericardium, heart, or coronary vasculature is treated or prevented. U.S. Pat. No. 6,333,347 discloses, at columns 1 et seq, As noted above, the present invention provides methods for treating or preventing disease of the pericardium, heart, or coronary vasculature (e.g., stenosis, restenosis, or atherosclerosis), comprising the step of administering to the pericardium, heart or, coronary vasculature an anti-microtubule agent. Briefly, a wide variety of anti-microtubule agents may be delivered, either with or without a carrier (e.g., a polymer or ointment), in order to treat or prevent disease. Representative examples of such agents include taxanes (e.g., paclitaxel (discussed in more detail below) and docetaxel) (Schiff et al., Nature 277: 665-667, 1979; Long and Fairchild, Cancer Research 54: 4355-4361, 1994; Ringel and Horwitz, J. Natl. Cancer Inst. 83(4): 288-291, 1991; Pazdur et al., Cancer Treat. Rev. 19(4): 351-386, 1993), campothecin, eleutherobin (e.g., U.S. Pat. No. 5,473,057), sarcodictyins (including sarcodictyin A), epothilones A and B (Bollag et al., Cancer Research 55: 2325-2333, 1995), discodermolide (ter Haar et al., Biochemistry 35: 243-250, 1996), deuterium oxide (D2 O) (James and Lefebvre, Genetics 130(2): 305-314, 1992; Sollott et al., J. Clin. Invest. 95: 1869-1876, 1995), hexylene glycol (2-methyl-2,4-pentanediol) (Oka et al., Cell Struct. Funct. 16(2): 125-134, 1991), tubercidin (7-deazaadenosine) (Mooberry et al., Cancer Lett. 96(2): 261-266, 1995), LY290181 (2-amino-4-(3-pyridyl)-4H-naphtho(1,2-b)pyran-3-cardonitrile) (Panda et al., J. Biol. Chem. 272(12): 7681-7687, 1997; Wood et al., Mol. Pharmacol. 52(3): 437-444, 1997), aluminum fluoride (Song et al., J. Cell. Sci. Suppl. 14: 147-150, 1991), ethylene glycol bis-(succinimidylsuccinate) (Caplow and Shanks, J. Biol. Chem. 265(15): 8935-8941, 1990), glycine ethyl ester (Mejillano et al., Biochemistry 31(13): 3478-3483, 1992), nocodazole (Ding et al., J. Exp. Med. 171(3): 715-727, 1990; Dotti et al., J. Cell Sci. Suppl. 15: 75-84, 1991; Oka et al., Cell Struct. Funct. 16(2): 125-134, 1991; Weimer et al., J. Cell. Biol. 136(1), 71-80, 1997), cytochalasin B (Illinger et al., Biol. Cell 73(2-3): 131-138, 1991), colchicine and CI 980 (Allen et al., Am. J. Physiol. 261(4 Pt. 1) L315-L321, 1991; Ding et al., J. Exp. Med. 171(3): 715-727, 1990; Gonzalez et al., Exp. Cell. Res. 192(1): 10-15, 1991; Stargell et al., Mol. Cell. Biol. 12(4): 1443-1450, 1992; Garcia et al., Antican. Drugs 6(4): 533-544, 1995), colcemid (Barlow et al., Cell. Motil. Cytoskeleton 19(1): 9-17, 1991; Meschini et al., .J Microsc. 176(Pt. 3): 204-210, 1994; Oka et al., Cell Struct. Funct. 16(2): 125-134, 1991), podophyllotoxin (Ding et al., J. Exp. Med 171(3): 715-727, 1990), benomyl (Hardwick et al., J. Cell. Biol. 131(3): 709-720, 1995; Shero et al., Genes Dev. 5(4): 549-560, 1991), oryzalin (Stargell et al., Mol. Cell. Biol. 12(4): 1443-1450, 1992), majusculamide C (Moore, J. Ind. Microbiol. 16(2): 134-143, 1996), demecolcine (Van Dolah and Ramsdell, J. Cell. Physiol. 166(1): 49-56, 1996; Wiemer et al., J. Cell. Biol. 136(1): 71-80, 1997), methyl-2-benzimidazolecarbamate (MBC) (Brown et al., J. Cell. Biol. 123(2): 387-403, 1993), LY195448 (Barlow & Cabral, Cell Motil. Cytoskel. 19: 9-17, 1991), subtilisin (Saoudi et al., J. Cell Sci. 108: 357-367, 1995), 1069C85 (Raynaud et al., Cancer Chemother. Pharmacol. 35: 169-173, 1994j, steganacin (Hamel, Med Res. Rev. 16(2): 207-231, 1996), combretastatins (Hamel, Med Res. Rev. 16(2): 207-231, 1996), curacins (Hamel, Med Res. Rev. 16(2): 207-231, 1996), estradiol (Aizu-Yokata et al., Carcinogen. 15(9): 1875-1879, 1994), 2-methoxyestradiol (Hamel, Med Res. Rev. 16(2): 207-231, 1996), flavanols (Hamel, Med Res. Rev. 16(2): 207-231, 1996), rotenone (Hamel, Med Res. Rev. 16(2): 207-231, 1996), griseofulvin (Hamel, Med Res. Rev. 16(2): 207-231, 1996), vinca alkaloids, including vinblastine and vincristine (Ding et al., J. Exp. Med 171(3): 715-727, 1990; Dirk et al., Neurochem. Res. 15(11): 1135-1139, 1990; Hamel, Med Res. Rev. 16(2): 207-231, 1996; Illinger et al., Biol. Cell 73(2-3): 131-138, 1991; Wiemer et al., J. Cell. Biol. 136(1): 71-80, 1997), maytansinoids and ansamitocins (Hamel, Med Res. Rev. 16(2): 207-231, 1996), rhizoxin (Hamel, Med Res. Rev. 16(2): 207-231, 1996), phomopsin A (Hamel, Med. Res. Rev. 16(2): 207-231, 1996), ustiloxins (Hamel, Med Res. Rev. 16(2): 207-231, 1996), dolastatin 10 (Hamel, Med. Res. Rev. 16(2): 207-231, 1996), dolastatin 15 (Hamel, Med. Res. Rev. 16(2): 207-231, 1996), halichondrins and halistatins (Hamel, Med. Res. Rev. 16(2): 207-231, 1996), spongistatins (Hamel, Med Res. Rev. 16(2): 207-231, 1996), cryptophycins (Hamel, Med. Res. Rev. 16(2): 207-231, 1996), rhazinilam (Hamel, Med. Res. Rev. 16(2): 207-231, 1996), betaine (Hashimoto et al., Zool. Sci. 1: 195-204, 1984), taurine (Hashimoto et al., Zool. Sci. 1: 195-204, 1984), isethionate (Hashimoto et al., Zool. Sci. 1: 195-204, 1984), HO-221 (Ando et al., Cancer Chemother. Pharmacol. 37: 63-69, 1995), adociasulfate-2 (Sakowicz et al., Science 280: 292-295, 1998), estramustine (Panda et al., Proc. Natl. Acad. Sci. USA 94: 10560-10564, 1997), monoclonal anti-idiotypic antibodies (Leu et al., Proc. Natl. Acad. Sci. USA 91(22): 10690-10694, 1994), microtubule assembly promoting protein (taxol-like protein, TALP) (Hwang et al., Biochem. Biophys. Res. Commun. 208(3): 1174-1180, 1995), cell swelling induced by hypotonic (190 mosmol/L) conditions, insulin (100 nmol/L) or glutamine (10 mmol/L) (Haussinger et al., Biochem. Cell. Biol. 72(1-2): 12-19, 1994), dynein binding (Ohba et al., Biochim. Biophys. Acta 1158(3): 323-332, 1993), gibberelin (Mita and Shibaoka, Protoplasma 119(1/2): 100-109, 1984), XCHO1 (kinesin-like protein) (Yonetani et al., Mol. Biol. Cell 7(suppl): 211A, 1996), lysophosphatidic acid (Cook et al., Mol. Biol. Cell 6(suppl): 260A, 1995), lithium ion (Bhattacharyya and Wolff, Biochem. Biophys. Res. Commun. 73(2): 383-390, 1976), plant cell wall components (e.g., poly-L-lysine and extensin) (Akashi et al., Planta 182(3): 363-369, 1990), glycerol buffers (Schilstra et al., Biochem. J. 277(Pt. 3): 839-847, 1991; Farrell and Keates, Biochem. Cell. Biol. 68(11): 1256-1261, 1990; Lopez et al., J. Cell. Biochem. 43(3): 281-291, 1990), Triton X-100 microtubule stabilizing buffer (Brown et al., J. Cell Sci. 104(Pt. 2): 339-352, 1993; Safiejko-Mroczka and Bell, J. Histochem. Cytochem. 44(6): 641-656, 1996), microtubule associated proteins (e.g, MAP2, MAP4, tau, big tau, ensconsin, elongation factor-1-alpha (EF-1.alpha.) and E-MAP-115) (Burgess et al., Cell Motil. Cytoskeleton 20(4): 289-300, 1991; Saoudi et al., J. Cell. Sci. 108(Pt. 1): 357-367, 1995; Bulinski and Bossler, J. Cell. Sci. 107(Pt. 10): 2839-2849, 1994; Ookata et al., J. Cell Biol. 128(5): 849-862, 1995; Boyne et al., J. Comp. Neurol. 358(2): 279-293, 1995; Ferreira and Caceres, J. Neurosci. 11(2): 392-400, 1991; Thurston et al., Chromosoma 105(1): 20-30, 1996; Wang et al., Brain Res. Mol. Brain Res. 38(2): 200-208, 1996; Moore and Cyr, Mol. Biol. Cell 7(suppl): 221-A, 1996; Masson and Kreis, J. Cell Biol. 123(2), 357-371, 1993), cellular entities (e.g., histone H1, myelin basic protein and kinetochores) (Saoudi et al., J. Cell. Sci. 108(Pt. 1): 357-367, 1995; Simerly et al., J. Cell Biol. 111(4): 1491-1504, 1990), endogenous microtubular structures (e.g., axonemal structures, plugs and GTP caps) (Dye et al., Cell Motil. Cytoskeleton 21(3): 171-186, 1992; Azhar and Murphy, Cell Motil. Cytoskeleton 15(3): 156-161, 1990; Walkeret al., J. Cell Biol. 114(1): 73-81, 1991; Drechsel and Kirschner, Curr. Biol. 4(12): 1053-1061, 1994), stable tubule only polypeptide (e.g., STOP145 and STOP220) (Pirollet et al., Biochim. Biophys. Acta 1160(1): 113-119, 1992; Pirollet et al., Biochemistry 31(37): 8849-8855, 1992; Bosc et al., Proc. Natl. Acad. Sci. USA 93(5): 2125-2130, 1996; Margolis et al., EMBO J. 9(12): 4095-4102, 1990) and tension from mitotic forces (Nicklas and Ward, J. Cell Biol. 126(5): 1241-1253, 1994), as well as any analogues and derivatives of any of the above. Such compounds can act by either depolymerizing microtubules (e.g., colchicine and vinblastine), or by stabilizing microtubule formation (e.g., paclitaxel).”

Formation of a Charged Tubulin Assembly

Referring again to FIGS. 7, 8A, and 8B, in step 438 the “charged tubulin dimer” (see dimer 435 in FIG. 8B) is allowed to polymerize until it has reached at least 90 percent of completion; the extent of desired completion of polymerization may be determined by, e.g., turbidity meter 426. The charged assembly 439 thus produced preferably has a desired set of properties and may be used, e.g., a biological entity 302. Alternatively, the charged assembly 439 may be further reacted with other reagents to form charged assembly 441, which also may be used as entity 302.

The biological entity 302 preferably contains at least about 90 weight percent of tubulin and, more preferably, at least about 95 weight percent of tubulin. As used herein, the term tubulin refers to monomeric tubulin (such as, e.g., alpha-tubulin, beta-tubulin, gamma-tubulin, etc.), dimeric tubulin (such as, e.g., a heterodimeter of tubulin made from alpha-tubulin and beta-tubulin), protofilaments made from tubulin, microtubules and/or microtubular fragments made from tubulin, and the polymorphic tubulin assemblies referred to elsewhere in this specification.

Referring again to FIG. 8B, the reaction product 441 is comprised of a positive (P) section comprised of positively charged tubulin dimers. It is preferred that this “P section” have a molecular weight of at least about 1,000 Daltons and, preferably, at least about 5,000 Daltons. In another embodiment, this “P” section has a molecular weight of at least about 10,000 Daltons and, more preferably, at least about 15,000 Daltons. In an even more preferred embodiment, such “P” section has a molecular weight of at least about 30,000 Daltons and, more preferably, at least about 40,000 Daltons. In one embodiment, the molecular weight of the charged moiety is at least about 1,000,000 Daltons.

Referring again to FIG. 8B, it will be seen that the “P section” of moiety 439 is identified as positive section 443, whereas the “N” (negative”) section of moiety 441 is identified as section 445. The comments regarding the molecular weight of the “N section” are equally applicable to the “P” section.

The “N section” may be formed by repeating steps 430/434/438 by, instead of using “positively charged tubulin” in step 434, using negatively charged tubulin (identified as element 437) and allowing such negatively charged tubulin to polymerize until the desired degree of polymerization, as evidenced by turbidity meter 426, has occurred.

In one emobidment, illustrated in FIGS. 8A and 8B, a mixture of positively charged and negatively charged tubulin is used in step 430 to produce a charged region (either N or P) whose charge will depend upon which tubulin dimer predominates in the reaction mixture 428.

It is preferred that each of the P moieties 443 and the N moieties 445 have a bulk electrical conductivity of at least 10⁻⁷ ohm⁻¹ meter⁻¹ Siemens and, preferably from about 10⁻⁷ ohm⁻¹ meters⁻¹ Siemens to about 10⁸ ohm⁻¹ meter⁻¹ Siemens. In one preferred embodiment, such bulk conductivity is from about 10⁻⁷ ohm⁻¹ meter⁻¹ Siemens to about 10⁻² ohms⁻¹ meter⁻¹ Siemens.

One may measure the bulk electrical conductivity of the P moieties 443 and the N moieties 445 by conventional means such as, e.g. the means disclosed at pages 179-181 of J. S. Balkemore's “Solid State Physics,” W.B. Saunders Company, Philadelphia, Pa., 1969; the measurement is preferably made at ambient temperature. Reference also may be had, e.g., to U.S. Pat. No. 3,604,108, the entire disclosure of which is hereby incorporated by reference into this specification.

In one embodiment, each of the P moieties 443 and the N moieties 445 have a certain number of free charges disposed within it; the net polarity of the free charges will determine whether the moiety is a P or an N moiety. It is preferred that each of such N and P moieties have a free charge of from about 10¹² to 1025 elemental charges per cubic centimeter of such moiety. It should be noted that naturally occurring microtubules, at a pH of 7, comprise only negative charges. In one embodiment of this invention, a microtubule with at least one region of positive charges is provided. Referring to FIG. 8B, the positively charged region 443 is shown as part of the microtubule that is being formed. In one preferred embodiment, the positively charged region 443 has a length of at least 2 nanometers and a molecular weight of at least 1,000 Daltons. In one aspect of this embodiment, the positively charged region 443 has a length of at least 4 nanometers.

In one preferred embodiment, each of the P moiety and/or the N moiety has a molecular weight of at least about 30,000 Daltsons and has a concentration of elemental charges that is at least about 10¹⁴ elemental charges per cubic centimeter, and preferably at least about 10¹⁷ elemental charges per cubic centimeter. In one embodiment, such P moiety and/or such N moiety has a concentration of elemental charges of at least 10¹⁸ elemental charges per cubic centimeter and, more preferably, at least 10¹⁹ elemental charges per cubic centimeter. In one embodiment, each such P moiety and/or such N moiety has a concentration of elemental charges of at least 10²⁰ elemental charges per cubic centimeter. The concentration of elemental charges is preferably measured at pH 7.

In one embodiment, the free charges present in the P moiety and the N moiety preferably have a specified degree of drift mobility. This drift mobility may be measured by conventional means, such as isoelectric focusing. This technique is discussed at page 254 of the aforementioned Stensch et al. reference, where it is described as “An electrophoretic technique for fractionating amphoteric molecules, particularly, proteins, that is based on their distrubiton in a pH gradient under the influence of an electric field that is applied across the gradient. The molecules distribute themselves in the gradient according to their isoelectric pH values. Positvely charged proteins are repelled by the anode and negatively charged proteins are repelled by the cathode: consequently, a given protein moves in the pH gradient and binds at a point where the pH of the gradient equals the isoelectric pH of the prtein. The pH gradient is produced in a chromatographic column by the electrolysis of amphoteric compounds and is stabilized by either a density gradient or a gel.” Reference also may be had, e.g., to U.S. Pat. No. 3,915,839 (apparatus for isolectric focusing), U.S. Pat. No. 3,951,777 (isoelectric focusing devices), U.S. Pat. No. 3,962,058 (flat bed isoelectric focusing devices), U.S. Pat. No. 4,204,929 (isoelectric focusing method), U.S. Pat. No. 4,312,739 (medium for isoelectric focusing), U.S. Pat. No. 4,362,612 (isoelectric focusing apparatus), U.S. Pat. No. 4,441,978 (separation of proteins using electrodialysis—isoelectric focusing combaintion), U.S. Pat. No. 4,481,141 (device for isoelectric focusing), U.S. Pat. No. 4,588,492 (rotating apparatus for isoelectric focusing), U.S. Pat. No. 4,670,119 (isoelectric focusing device and process), U.S. Pat. No. 4,673,483 (isoelectric focusing apparatus), U.S. Pat. No. 4,963,236 (apparatus and methods for isoelectric focusing), U.S. Pat. No. 4,971,670 (isoelectric focusing process and means for carrying out said process), U.S. Pat. No. 5,082,548 (isoelectric focusing apparatus), U.S. Pat. No. 5,376,249 (analysis utilizing isoelectric focusing), U.S. Pat. No. 5,468,359 (method of determining presence of an analyate by isoelectric focusing), U.S. Pat. No. 5,866,683 (isoelectric point markers for isoelectric focusing with fluorescence detection), U.S. Pat. No. 6,572,751 (method and apparatus for continous flow isoelectric focusing for purifying biological substances), U.S. Pat. No. 6,638,408 (method and device for separation of charged molecules by solution isoelectric focusing), and the like. The entire disclosure of each of these United States patents is hereby incorporated by reference into this specification.

It is preferred that the free charges present in the P moiety and the N moiety have a drift mobility of at least about 10 cm²/volt/second and, preferably, at least about 50 cm²/volt/second. In one embodiment, the free charges have a drift mobility of at least about 100 cm²/volt/second and, more preferably, at least about 1000 cm²/volt/second. In yet another embodiment, the free charges have a drift mobility of at least about 5000 cm²/volt/second.

As is known to those skilled in the art, drift mobility is the increase in the average velocity of a charge carrier per electric field intensity. Reference may be had, e.g., to U.S. Pat. No. 4,319,187 (“Method for measuring the drift mobility in doped semiconductors”), the entire disclosure of which is hereby incorporated by reference into this specification. Reference may also be had to equation 3-18 of page 148 of J. S. Blakemore's “Solid State Physics,” W.B. Saunders Company, Philadelphia, Pa., 1969.

Referring again to FIGS. 7, 8A, and 8B, the P moiety 443 is allowed to assemble until at least about 90 weight percent of the of the P tubulin dimer has so assembled. During this process, one may mix the reaction mixture with a mixer 445. One may also add agents via line 436 that facilitate such self-assembly such as, e.g., guanosine triphosphate, magnesium salt (such as magnesium chloride), standard buffers, etc.; many of these reagents are described elsewhere in this specification.

When at least about 90 weight percent of the P dimer 435 has self-assembled, an N-type dimer 437 is preferably added and reacted using substantially the same reaction conditions as were used, e.g., in step 438 (see FIG. 7). The N-type dimer 437 will add onto the end of the growing microtubule 450 (see FIG. 8B). After the turbidity meter 426 indicates that the polymerization of the dimer 437 is substantially complete, one may then add another reagent.

The other reagent may be another P dimer (so that one may form a PNP structure), and/or it may be one or more of the other reagents specified elsewhere in this specification.

As will be apparent to those skilled in the art, the process depicted in FIGS. 8A and 8B is a dynamic one, with one being able to (a) remove reaction mixture 428 at any desired time, and isolate and/purify components of such reaction mixture, (b) add new reagents at any time, (c) recycle reaction products that have been withdrawn from the reaction mixture, either before or after they have been separated and/or purified and/or modified, and (d) synthesize substantially any desired structure. Thus, steps 412, 414, 416, 418, 424, 430, 4343, and/or 438 may be repeated and/or modified with the same or different reagents to make many different types of microtubule structure.

Referring again to FIG. 7, and in step 444 thereof, one may cap the growing microtubule assembly and terminate its growth by conventional means. One may use any conventional means of filament capping to terminate the growth of the microtubule assembly at its “plus end.” Reference may be had, e.g., to U.S. Pat. No. 4,857,538 (new compounds for the study and treatment of microfilament organization in cells), U.S. Pat. Nos. 5,783,662, 5,798,380 (cytoskeletal active agents for glaucoma therapy), U.S. Pat. Nos. 6,114,118, 6,586,425, and 6,716,597 (methods and products for regulating cell motility); the entire disclosure of each of these United States patents is hereby incorporated by reference into this specification. Reference also may be had, e.g., to pages 937-938 of Bruce Alberts et al.'s “Molecular Biology of the Cell,” Fourth Edition (Garland Science, New York, N.Y., 2002).

By way of illustration and not limitation, one may cap the growing microtubule assembly with Vinblastine in step 444 to form a cap 447 at the growing end of the microtubule. As is known to those skilled in the art, the addition of gamma tubulin (in step 418 of FIG. 7) “capped” the “minus end” of the growing microtubule assembly 439.

After the plus end of the microtubule assembly is capped, one my purify the completed microtubule in step 413 by conventional means.

FIG. 9 is a schematic illustration of various charged microtubule assemblies (500, 502, 504, 506, 508) that may be made with tubulin dimer 510 with a negative charge, tubulin dimer 512 with a positive charge, and neutral tubulin dimer 514 using the processes of FIGS. 7, 8A, and 8B.

Preparation of Conductive Biological Links

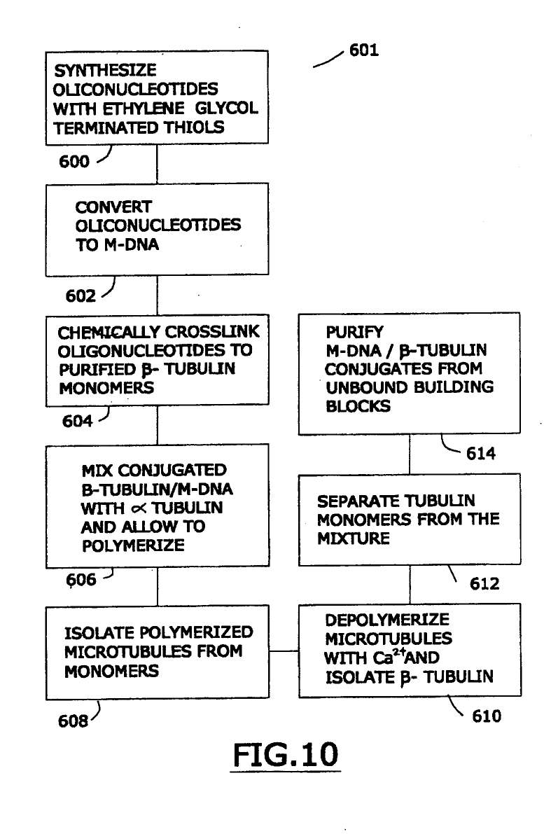

FIG. 10 illustrates a process for preparing a microtubule containing one or more conductive polymeric links. These type of conductive links are discussed in an article by A. Rakitin et al. entitled “Metallic Conduction through Engineered DNA: DNA Nanoelectronic Buidling Blockis,” Volume 86, Number 16, Physical Review Letters, Apr. 16, 2001, pages 3670-3673.

The Rakitin et al. article provides a process for modifying the conductivity of DNA molecules by substituting the imino proton of each base pair with a metal ion; the composition so produced was about 15 microns long and was called M-DNA.

As is disclosed at page 3670 of the Rakitin et al. article, “Four types of . . . DNA samples were prepared for conductivity measurements . . . M-DNA was prepared in 0.1 nM Zn²⁺ at pH 9.0 [10,11] and placed across the gap as above. The “10,11” references were P. Aich et al. (J. Mol. Biol. 294, 477, 1999), and J. S. Lee et al. (Biochem. Cell Biol. 71, 162, 1993), respectively.

At page 3672 of the Rakitin et al. article, it was stated that “The evidence of metalliclike conduction through M-DNA is found . . . . The ability to convert normal DNA into M-DNA and the resultant drastic cange of DNA conductivity oens up a whole new range of opportunities for molecular electronic engineering, and provide us a new degree of freedom in molecular electronics and sensor designs.”

FIG. 10 is a flow diagram illustrating a preferred process for preparing a microtubule that contains one or more conductive links, such as those described in the Rakitin article.

Referring to FIG. 10, an M-DNA is prepared in step 602. The M-DNA has the structure described in the aforementioned Rakitaiin et al. article, and it is made in accordance with the Aich et al. and Lee et al. articles cited in references 10 and 11 of the Rakitin article.

It is preferred to use thiolated, single-stranded DNA to prepare single-stranded probe sequences. In step 600, oligonucleotides with oligo (ethylene glycol) terminated thiols are prepared. These oligo (ethylene glycol) terminated thiols are well known to those skilled in the art and may be prepared, e.g., in accordance with the teachings of U.S. Pat. No. 6,114,513; the entire disclosure of this United States patent is hereby incorporated by reference into this specification.

As is disclosed in such U.S. Pat. No. 6,114,513, “This invention provides nucleosides, oligonucleotides and oligonucleosides containing alkylthiol chemical functionality. The nucleoside subunits can be “natural” or “synthetic” moieties. Each nucleoside is formed from a naturally occurring or synthetic base and a naturally occurring or synthetic pentofuranosyl sugar group.”

U.S. Pat. No. 6,114,513 also discloses that “The term “oligonucleotide” refers to a polynucleotide formed from a plurality of linked nucleotide units. The nucleotide units each include a nucleoside unit. In the context of this invention, the term “oligonucleoside” refers to a plurality of nucleoside units that are linked together. In a generic sense, since each nucleotide unit of an oligonucleotide includes a nucleoside therein, the term “oligonucleoside” can be considered to be inclusive of oligonucleotides (i.e., nucleosides linked together via phosphate linking groups). In a further sense, the term “oligonucleoside” also refers to a plurality of nucleosides that are linked together via linkages other than phosphate linkages. The term “oligonucleoside” thus effectively includes naturally occurring species or synthetic species formed from naturally occurring subunits. For brevity, the term “oligonucleoside” will be used to denote both phosphate linked (oligonucleotides) and non-phosphate linked polynucleoside species.”

U.S. Pat. No. 6,114,513 also discloses that “Oligonucleosides according to the invention also can include modified subunits. Representative modifications include modification of a heterocyclic base portion of a nucleoside or a sugar portion of a nucleoside. Exemplary modifications are disclosed in the following U.S. patent application Ser. No. 07/463,358, filed Jan. 11, 1990, now abandoned entitled Compositions And Methods For Detecting And Modulating RNA Activity; Ser. No. 07/566,977, filed Aug. 13, 1990, now abandoned, entitled Sugar Modified Oligonucleotides That Detect And Modulate Gene Expression; Ser. No. 07/558,663, filed Jul. 27, 1990, now U.S. Pat. No. 5,138,045, entitled Novel Polyamine Conjugated Oligonucleotides; Ser. No. 07/558,806, filed Jul. 27, 1991, now abandoned, entitled Nuclease Resistant Pyrimidine Modified Oligonucleotides That Detect And Modulate Gene Expression and Serial No. PCT/US91/00243, filed Jan. 11, 1991, entitled Compositions and Methods For Detecting And Modulating RNA Activity. Each of these patent applications are assigned to the assignee of this invention. The disclosure of each is incorporated herein by reference.”

U.S. Pat. No. 6,114,513 also discloses that “The term oligonucleoside thus refers to structures that include modified portions, be they modified sugar moieties or modified base moieties, that function similarly to natural bases and natural sugars. Representative modified bases include deaza or aza purines and pyrimidines used in place of natural purine and pyrimidine bases; pyrimidines having substituent groups at the 5 or 6 position; and purines having altered or replacement substituent groups at the 2, 6 or 8 positions. Representative modified sugars include carbocyclic or acyclic sugars, sugars having substituent groups at their 2′ position, and sugars having substituents in place of one or more hydrogen atoms of the sugar. Other altered base moieties and altered sugar moieties are disclosed in U.S. Pat. No. 3,687,808 and PCT application PCT/US89/02323.”

Example 1 of U.S. Pat. No. 6,114,513 is instructive, and it illustrates the preparation of a “Compound 1,” S-Trityl-6-mercaptohexylbromide, 1,1′,1″-{{(6-bromohexyl)thio]methylidyne]trisbenzene (Compound 1). As is disclosed in this Example 1, “To a solution of triphenylmethanethiol (Fluka; 69 g, 250 mmol) in 500 mL 95% ethanol (EtOH) was added 11 grams of sodium hydroxide dissolved in 75 mL of water (275 mmol). After stirring for about 15 minutes in argon atmosphere, using an addition funnel, 1,6-dibromohexane (91.5 g, 375 mmol, 58 mL) dissolved in 100 mL of 95% EtOH was added dropwise over a period of 1 hour with vigorous stirring. After about 15 minutes of stirring of addition, a brown white solid separates out from the reaction flask. After stirring for additional 4 hours, the reaction mixture was filtered. The filtrate was evaporated under high vacuum and the oily residue was combined with the filtered residue and dissolved in 500 mL CH₂Cl₂, filtered again, the filtrate was washed once with water (200 mL) and once with saturated NaCl solution. After drying the CH₂Cl₂ layer over MgSO4, it was concentrated to 200 mL in volume. About 200 mL of hexane was added and the solution was left in freezer. Three crops of cream white product was isolated out. Total yield 81 g (184 mmol, 73% yield). After one more recrystallization the product melted at 91-92° C.”

U.S. Pat. No. 6,114,513 also discloses (in Example 1) that “Portions of the product are independently treated with sodium cyanide followed by hydrolysis to give the corresponding acid, S-trityl-6-mercaptohexanoic acid (Compound 2), with lithium azide followed by triphenylphosphine reduction to give the corresponding amine, S-trityl-6-mercapto hexylamine (Compound 3), and with sodium hydrogen sulfide to give the corresponding thiol, (1-S-trityl-thio-hexylmercaptan).”

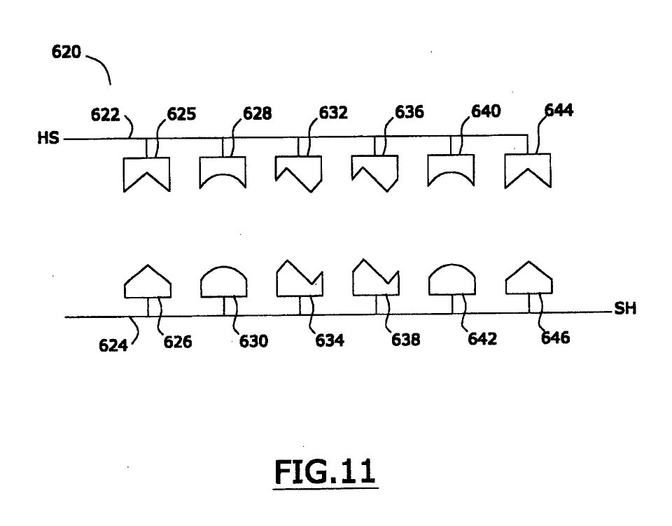

FIG. 11 is a schematic illustration of a typical thiolated oligonucleotide assembly 620 comprised of thiolated oligonucleotide 622 and thiolated oligonucletoide 624. In these oligonucelotides, the thiol(SH) is preferably bonded to the 5′ end. In the embodiment, depicted, each of oligonucleotide 622 and 624 is comprised of bases that are complementary to each other. Thus, base 625 is complementary 5o base 626, base 628 is complementary to base 630, base 632 is complementary to base 634, base 640 is complementary to base 642, and base 644 is complentary to base 646. These complementary base pairs are preferably chosen to correspond to the M-DNA discussed in the Rakitin et al. article.

Referring again to FIG. 10, and in step 602 thereof, the thiol-terminated oligonucleotides are converted into M-DNA. It is preferred to form such M-DNA at pH conditions above 8 in the presence of zinc ion and/or nickel ion and/or cobalt ion; it is preferred not to sue calcium ion or magnesium ion. All bacterial and synthetic DNA usually dismutates to M-DNA under these conditions, but the process is readily reversible with the addition of EDTA.

Referring again to FIG. 10, and to step 604 thereof, DNA/beta-tubulin monomer conjugates are preferably formed by chemically crosslinking the aforementioned thiolated single-stranded DNA. In one preferred embodiment, beta tubulin (5 milligrams per milliliter) is reacted with a ten-fold excess of sulfosuccinimidyl 4-(p-maleimidophenyl) butyrate in PBS (100 millimolar phosphate buffer, pH 7.4, 150 millimolar NaCl). After incubation for 30 minutes at room temperature, the derivatized tubulin is preferably desalted by ultrafiltraiton (using a 30,000 molecular weight cutoff membrane), and the buffer is changed to phosphate buffered EDTA (100 millimolar phosphate buffer at pH 6.8, 5 millimolar of EDTA). Excess thiolated DNA is preferably removed by ultrfiltration (100,000 molecular weight cutoff), and the purification is preferably verified by non-denaturing PAGE.

Referring again to FIG. 10, and in step 606 thereof, the purified product obtained in step 604 is added to a reaction veseel comprised of alpha-tubulin monomers (in an equimolar concentration), and GTP is added in an amount sufficient to induce the polymerization of the monomers into microtubules. In one embodiment, the reaction is conducted under ambient conditions for about 1 hour until it has gone to about 50 percent of completion.

In step 608, the tubulin assemblies produced in step 606 (such as, e.g., microtubules) are isolated from the reaction mixture and purified by conventional means such as, e.g., size exclusion protein column chromatography.

In step 610, the tubulin assemblies isolated in step 608 are depolymerized to form tubulin monomers. One may conventional tubulin depolymerizing processes such as, e.g., those described elsewhere in this specification. Thus, e.g., one may use calcium ion provided, e.g., by soluble calcium chloride. This depolymerization step produces a mixture of tubulins.

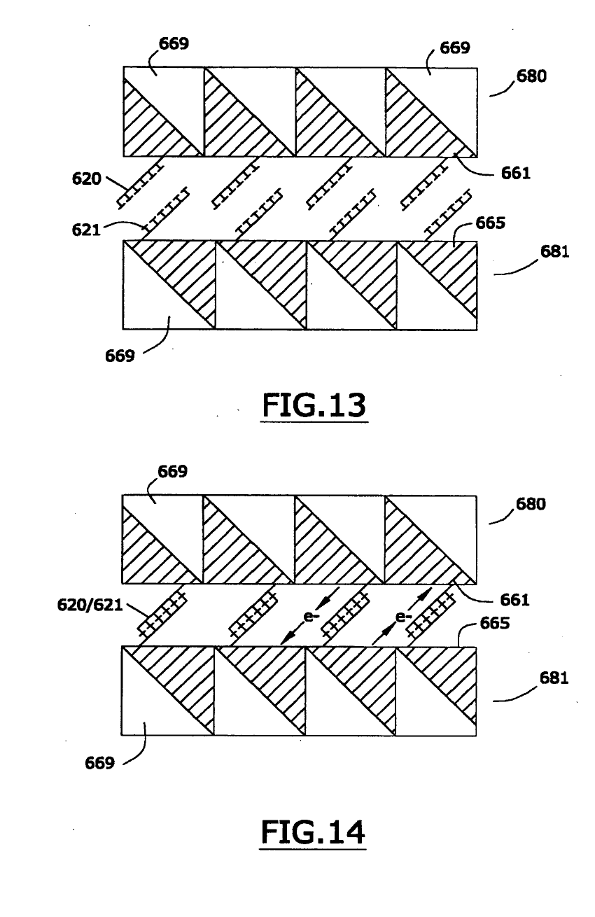

The mixture of tubulins produced in step 610 is then separated into its individual components in step 112 using conventional means such as, e.g., size column chromatography or electrolpheris. One preferred component obtained in this step is illustrated in FIG. 12, as tubulin construct 660; another is illustrated in FIG. 12 as tubulin construct 662. The tubulin construct 660 is comprised of a beta-tubulin portion 661 and oligonucleotide portion 663. The tubulin construct 662 is comprised of beta-tubulin portion 665 and oligonucleotide portion 667. As will be apparent to those skilled in the art, the tubulin constructs 660 and 662 will be likely to “self assemble” because of the complementarity of their base pairs (such as, e.g., base pairs 625/626). This is illustrated in FIG. 13.

When the beta-tubulin/M-DNA conjugate 660 (or 662) is incorporated into a biological polymer, such as a microtubule, the structure 680 illustrated in FIG. 13 (and also in FIG. 14) will be produced; such FIGS. 13 and 14 illustrate the binding of two tubulin portions of microtubules 680 and 681. Refering to such FIG. 13, and in the preferred embodiment depicted therein, it will be seen that microtubule assembly 680 is comprised of a multiplicity of beta tubulin/M-DNA conjugates with extending M-DNA “tails” 620. In the embodiment depicted in FIG. 13, the beta-tubulin conjugates 660 (see FIG. 12) have copolymerized with alpha-tubulins 669. Microtubules assembly 681 is similarly constituted.

Referring again to FIG. 13, and also to FIG. 14, it will be seen that when M-DNA “tail” 620 is near an M-DNA “tail” 621, the two tails will bind to each other through hydrostatic interactions, thereby effectively joining beta-tubulin 661 with beta tubulin 665. When the two tails 620/621 are physically attached to each other, the microtubles 680/681, in addition to being physically attached, are also electrically attached to each other by the conductive M-DNA segments 620/621.

As will be apparent, there are many different monomers, dimers, polymers, and fragements that an be made in accordance with the process illustrated in FIGS. 7 through 14.

The monomer may, e.g., be eithere an alpha-tubulin monomer or a beta-tubulin monomer. Each of these monomers may exist either by itself and/or linked to one or more condutive DNA segements.

The dimer may consist of or comprise two “N monomers”(NN), each of which has a net negative charge. Alternatively, the dimer may consist of or comprise two “P” monomers (“PP”), each of which has a net positive charge. Alternatively, the dimer may be an NP, a PN, an NPN, a PNP, or other monomer. As will be apparent, for each of such dimers, the monomers comprising such dimer may be either alpha-tubulin or beta-tubulin.

The dimer, additionally, may contain one or more conductive DNA links (L), such that structures like LNN, NNL, LPP, PLL, LNP, NPL, and the like, may be prepared and used.

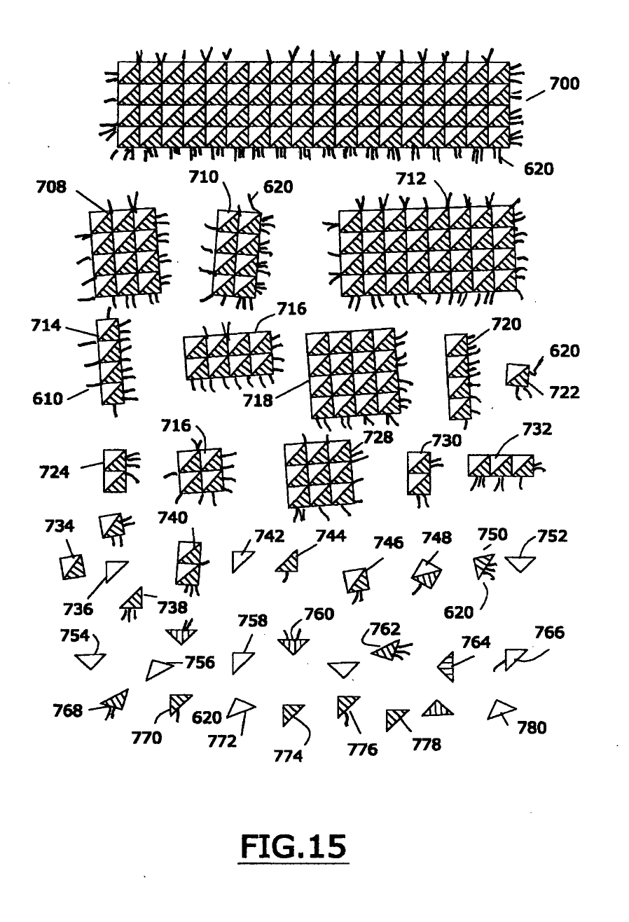

FIG. 15 illustrates some of the “building blocks” that may be made with the process of FIGS. 7 through 14. Referring to FIG. 15, and in the preferred embodiment depicted therein, one may may an assembled microtubule 700 that is comprised of, e.g., alpha-tubulin and beta tubulin M-DNA conjugates; in this Figure, the M-DNA conductive fragments are identified as element 620.

Referring again to FIG. 15, it will be seen that one can also make fragemented portions 702, 704, 706, 708, 710, 712, 714, 716, 718, 720, 722, 724, 726, 728, 730, 732, 734, 736, 738, 740, 742, 744, 746, 748, 750, 752, 754, 756, 758, 760, 762, 764, 766, 768, 770, 772, 774, 776, 778, and 780. One or more of these highly fragmented portions may be made from one or more of the lesser fragmented portions by conventional means such as, e.g., the addition of appropriate soluble calcium salts.

In one preferred embodiment, one or more of such fragments are alpha-tubulin monomers, such as monomer 742, that is not bound to a conductive DNA fragment 620.

In one preferred embodiment, one or more of such fragments is a beta-tubulin monomer, such as monomer 778, that is not bound to a conductive DNA fragment 620.

In one preferred embodiment, one or more of such fragments is a beta-tubulin monomer 764 that is bound to one or more conductive links 620, and/or an alpha tubulin monomer 766 that is bound to one or more conductive links 620.

Referring again to FIG. 15, and as will be apparent to those skilled in the art, some of such fragments are dimeric, trimeric, or polymeric in nature (such as, e.g., fragments 708, 710, 712, 714, 716, 718, and 720). As will be apparent one or more of these fragments 700 to 780 may be used in the processes described in FIGS. 7, 8A, and 8B and added to the reaction mixture 428 at any time during the process.

Metallization of One or More of the Fragments

An of the fragements illustrated in FIG. 15 may be metallized by conventional techniques and thereafter added to the reaction mixture 428. In one preferred embodiment, the procedure of R. Kirsch et al. is utilized.

In 1997 R. Kirsch et al. published an artilce on the “Three-dimensional metallization of microtubules” in Thin Solid Films 305 (1997), at pages 248-255. This article discussed “three-dimensional metallization of these MTs . . . by an electroless depositon technique of nickel initiated by molecular palladium catalysts.”

As was ntoe4d on page 248 of the Kirsh et al. article, the process of Kirsch et al. was applicable to many complex, three-dimensional structures. The authors noted that “Apart from mikcroelectronics applcications, three-dimensional nanostrutures will open the way to future development of micromachines and robots,” citing an work by K. E. Dreschler, “Nanosystems, Molecular Machinery, Manufacturing, and Computation” (Wiley, New York, 1992). The authors then disclosed that “Biological templating is a novel and promising direction of this development. Here, three-dimensional biolgocial specimens with characteristic nanometer dimensions are employed as templates for the build-up of solid-state nanostructurues. In addition to the small dimensions, the high morphological reproducibility of self-assembled biological templates is very advantageous for nanometer fabrication.”

The process of the Kirsch et al. article is applicable to protein template surfaces. As is disclosed on page 248 of such articles, “In order to deposit an adherent, thin metal film onto protein template surfaces, we followed the method of electroless metal plating developed by Brenner and Ridell[2] for finishing metal surfaces.” The Brenner and Ridell article was published in Proc. Am. Electroplaters Soc. 33 (1946) 16; 34(1947) 156.

In further describing their process, Kirsch et al. state (at page 248) that “Electroless deposition occurs by a redox process, where the cation of the metal to be deposited is chemically reduced. The redox process of electroless depositon takes place only on appropriate catalystic surfaces. Theeafter, a noncatalytic substrate, such as the surface of a nonconductor, must be treated with a noble metal catalyst [3] before it can be meteallized by an electroless process.” The reference [3] cited by Kirsch et al. is a work by F. Pearlstein in Met. Finish. 53 (1955) 59.

In further describing their process, Kirsch et al. (at page 248) that “Surface catalysis is commonly accomplished by nonspecific adsorption of colloidal tin-palladium particles onto the electrically insulating surface. A subsequent treatement of the specimen in alkaline solutions dissolves the outer tin oxide cover and the remaining palladium particles act as catalytic nucleation sites [4].” The reference [4] cited by Kirsch et al. is U.S. Pat. No. 3,011,920 of C. R. Shipley, the entire disclosure of which is hereby incorporated by reference into this specification.

The Kirsch et al. article further discloses (at page 248) that “The first biomolecular template based tubular microstructures were fabricated utilizing phospholipids microtubules [5]. The reference [5] was an article by J. M. Schnur et al., “Thin Solid Films 152 (1987) 181. The Schnur et al. work was also extensively reported, and utilized, in the patent literature.

U.S. Pat. No. 5,492,696, the entire disclosure of which is hereby incorporated by reference into this specification, discloses a metallized microtubule in its claim 1, describing “1.A composition for effecting the controlled release of an active agent to an environment, comprising a tubule containing a solution, dispersion, or blend of an active agent in a carrier in the lumen thereof, wherein said tubule has an inner diameter of from 0.1 to 1 μm, a wall thickness of from 5 to 50 nm, an optional 200-2,000 nm thick metal coating on said wall, and a length of 1 μm to 1 mm, wherein said active agent is tetracycline and said carrier is a water-soluble epoxy resin, said composition providing a zero order or first order release rate of said active agent from said tubule for a period of at least 30 days.” At column 4 of this patent, it is disclosed that “The preparation of tubules is also discussed in an article by Schnur et al., “Lipid-based Tubule Microstructures”, Thin Solid. Films, 152, pp. 181-206, (1987) and the articles cited therein. That same article, in which one of the inventors is a co-author, also describes metal coating tubules and using them as microvials to entrap, transport and deliver polymeric reagents to a desired site.” In Example 1 of this patent, it is disclosed that “Unpolymerized tubules were produced from a mixed solvent system of 70% ethanol and 30% water by volume, with a lipid concentration of 2.5 mg/ml. The microtubules are formed at 27° C. and following formation are dialyzed against water at pH 1.0 in 0.1N HCL. A commercial palladium and tin catalyst (Shipley Co., Waterbury, Mass.) is used as received. Cuposit (Shipley Co., Waterbury, Mass.) a commercial electroless copper plating bath is used per the manufacturers instructions to copper plate the accelerated microtubules. Following the plating reaction the excess bath is removed and the tubules are filtered to remove excess water. A commercial freeze drying apparatus is used to dry the metallized microtubules to a powder. The desired active agent at saturation in the selected carrier is added slowly to the dry microstructures during which time the material is captured by the microstructures by capillary attraction. Exogenous material is removed by suspending the tubules in an excess of solvent and is followed by rapid filtration. These microstructures can again be dried or suspended in a diluent liquid.” It should be noted that a similar disclosure also appears in U.S. Pat. No. 6,280,759, the entire disclosure of which is hereby incorporated by reference into this specification.

Metallized microtubules are also described in U.S. Pat. No. 5,650,787, the entire disclosure of which is hereby incorporated by reference into this specification. As is disclosed at columns 4-5 of such patent, “Metallized microtubules, which are hollow tubule-shaped microstructures, are presently the preferred implementation within this category. The fabrication of these structures is described in Yager et al., “Formation of Tubules by a Polymerizable Surfactant”, Molecular Crystals Liquid Crystals, vol. 106, 1984, pages 371-381, while a process for the deposition of thin metal coatings onto the microtubules is described in Schnur et al., “Lipid-based Tubule Microstructures”, Thin Solid Films, vol. 152, 1987, pages 181-206. Microtubules with metal coatings such as nickel or permalloy can be aligned with either an electric or a magnetic field during the formation of the anisotropic solid polymer composite.”

Metallized microtubules are also discussed in U.S. Pat. No. 6,452,564, the entire disclosure of which is hereby incorporated by reference into this specification. As is disclosed at columns 4-5 of this patent, “In a preferred embodiment, the microtubules are formed from diacetylenic lipid (1,2 bis(tricosa-10,12-diynoyl)-sn-glycero-3-phosphocholine), or DC8,9PC. See, for example, A. N. Lagarkov and A. K. Sarychev, Phys. Rev. B 53, 6318 (1996) and F. Behroozi, M. Orman, R. Reese, W. Stockton, J. Calvert, F. Rachfold and P. Schoen, J. Appl. Phys. 68, 3688 (1990). The lipid is dissolved in alcohol at 50° C., water is added, and the temperature lowered to room temperature. The lipid self-assembles itself into microtubules and subsequently precipitates. The particles are rinsed and coated with a palladium catalyst and mixed with metal ions and reductants. In contact with the catalyst, the metal ions-are reduced to neutral metal on the surface of the microtubules and coat the structure with a conductive layer of metal of several tenths of a micron thickness. Several metal species are available for use in this process, but nickel and copper appear to be of greatest potential usefulness for the present invention. Once the microtubules have been metallized, they can be dried and subsequently mixed into a polymer matrix. The choice of polymer is dependent upon the properties desired for the resulting composite. Among the desirable properties are flexibility, strength, both chemical and environmental stability, and appropriate viscosity to properly disperse the metal powder.”

Referring again to the process disclosed in the Kirsch et al. article, and at page 248 thereof, it is further disclosed that “Markowitz et al. [6] found that the diameters of metallized lipid tubules dpened on the duration of dialysis conducted prior to metallization. The observed a distribution of diamters ranging from 100 up to 900 nm.” The Reference [6] cited in the Kirsch et al. article was published in Thin Solid Films 224 (1993) 242.

The metllization of proteinaceous filaments was then discussed in the Kircsch et al. article. At page 248 thereof, it was disclosed that “Metallization of proteinaceous tubules was first demonstrated by Pazirendeh et al. [7] using rhapidosomes as the templates. Rhapidosomes are found in certain bact eria. They have a well defined diameter of 25 nm, considerably less than those of phospholipids tubules, and an average length of about 400 nanometers.” The reference [7] cited by Kirsch et al. was published in Biomimetics 1 (1992) 41.

At page 248 of the Kirsch et al. article, the electroless metal plating of microtubules was discussed. It was disclosed that “In this paper we report on electroless metal plating of microtubules (MTs). MTs are cytoskeletal protein polymers. They form highly dynamic structures which may polymerize and depolymerize during their function, e.g., they form transport tracts for organelles in the cell and determine mainoy cellular architecture . . . . MTs are tubular protein filaments. Each tubule is formed by longitudinally7 arranged protofilaments, each about 4-5 nm in diameter. The protofilaments consist of about 8 nm long heterodimers polymerized head to tail . . . . The outer diameter of the MTs is 25 nm . . . .”

At page 249 of the Kirsch et al. article, it is also disclosed that “MTs have the advantage that they can be assembled in vitro to a length of several micrometers. On the other hand, both the process of self-assembly and the morphological stability of MTs are very sensitive to the chemical environment and to temperature. For example, MTs cannot withstand treatment in strong alkaline or acidic solutions nor temperature above 60 degrees C., as are commonly applied in electoless copper plating baths . . . We show here that these problems can be circumvented by carrying out electroless metal plating of MTs under conditions similar to those required for the assembly process, i.e., at a pH of about 7 and physiological temperatures. In a first step, the protein surface is activated by direct adsorption of molecular palladium catalysts (first demonstrated by Chow et al. [9] for rhapidosomes).” The reference [9] appeared in Nanostruct. Mater. 2 (1993) 495.

The Kirsch et al. article further disclosed (at page 249) that “In a second step, under both appropriate chemical conditions and temperatures, nickel is deposited onto activated MTs by applying electroless metallization baths based on dimethylamine borane as the reducing agent, as developed by Narcus [10] and Paunovic [11].” The references [10] and [11] were Electronics Symp. Plating 54 (1967)380, and Plat. Surf. Finish. 70(1983)62, respectively.

In section 2.1 of the Kirsch et al. article, entitled “Microtubule assembly,” the authors disclosed that “The MTs were isolated from porcine brain by three cycles of temperature-dependent disasemlby/reassembly [12]. Pure tubulin hetermodimer preparations were obtained by phosphocellulose column chromatography[13]. All experiments in this study started from a tubulin heterodimer preparation stored at −80 degrees C. in a buffer solution of 100 nM MES (2-morpholino-ethanesulfonic acid monohydrate 0, 1 mM EGTA (ethylene glycol-bis-(beta-aminoethyl)-tetra-acetic acid), and 0.5 mM MgCl₂. The protein concentrate was about 1 mg ml⁻¹. The MTs were assembled in vitro by adding 0.5 mM GTP (guanosin-5′-triphosphate) and 10 mM taxol (from Taxus brevifolia) and warming the sample to 37 degrees C. The MT formation was accompanied by turbidity measurements at 340 nm. The steady state level, at which the tubulin mass in the polymerized state shows no further increase, was usually observed after 10 min. Thereafter, the polymer solution was centrifuged for 30 min at 14500 g to separate the MTs from the unpolymerized tubulin. The supernatatant was discarded and replaced by the same volume of pure MES buffer at pH 6.4 and the pellet was resuspended.” The [12] and [13] references cited in the Kirsch et al. article were M. L. Shelanski et al., Proc. Nat. Acad. Sci. USA 70 (1973)765, and M. D. Weingarten et al., Proc. Nat. Acad. Sci. USA 72 (1976) 1858, respectively.

In section 2.2 of the Kirsch et al. article, “Activation and nickel palting of the microtubule surface,” The authors disclosed that “To activate the MT surface by adsorption of Pd catalyst particles, a volume of about 300 microliters of the assembled MT solution was treated with an equal volume of a fresh, saturated Pd(CH₃)COO)₂ solution for about 2 hours at room temperature (pH 6.2). The catalyzed MTs were then washed with MES buffer by ultrafiltraiton using a 300 kDa MW cut-off membrane filter. The pellet in the membrane filter was subsequently redispersed in about 500 microliters of MES buffer.”

The Kirsch et al. article further discloses (at page 249) that “For the nickel plating we used two slightly different metallization baths, with dimethylamine borane (DMAB) as the reducing agent. The two baths were prepared with analytical-grade reagents and deionized water. Electroless nickel ‘solution A’ [10] contined 50 g l⁻¹ Ni(CH₃COO)₂ 6H₂O, 25 g l⁻¹ sodium citrate, 25 g l⁻¹ 85% lactic acic aq. sol. and 2.5 g l⁻¹ DMAB, whereas “solution B” [11] contained 39.4 g l⁻¹ NiSO₄.6H₂O, 20 g l⁻¹ sodium citrate, 10 g l⁻¹ 85% lactic acic aq. sol. and 4 g l⁻¹ DMAB. In both cases the pH was adjusted with NH₄OH.” The references [10] and [11] are described elsewhere in this specification.

The Kirsch et al. reference further discloses (at page 249) that “The Pd-activated MT preparation was mixed with an equal volume of the metallization bath. After 1 min, black metallized MTs settled at the bottom. The metallization process was usually stopped by decreasing the concentration of the metllization bath by at least a factor of 100. The metallized MTs were then washed and stored in water.”

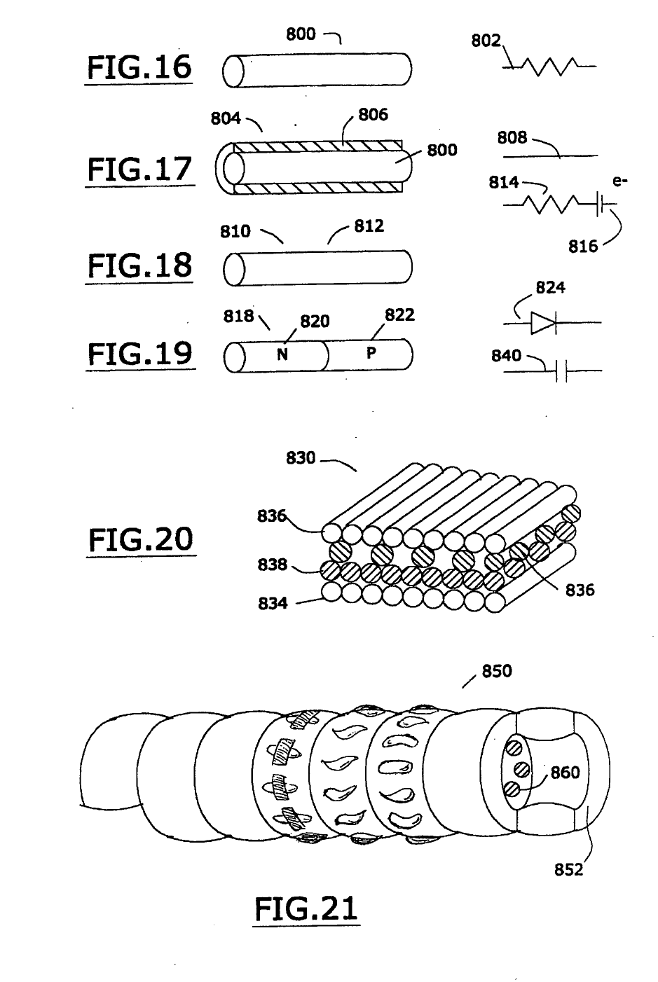

FIG. 16 et seq. illustrates some of the “building blocks” that can be made by the process of this invention and that may be used as electrical components.

FIG. 16 illustrates a microtubule 800 without any net charge that is equivalent to a resistor 802 with a resistance of about 200 kilohms per micrometer of length.

FIG. 17 illustrates a metallized microtubule 904 that is comprised of metal 806 and microtubule 800. This is equivalent to a conductor 808 with a conductivity, when gold is used, of about 10⁷ ohms⁻¹ meters⁻¹ Siemens.

FIG. 18 illustrates a microtubule 810 comprised of a charge 812 that acts a resistor 814 in series with a power source 816.

FIG. 19 illustrates a microtubule assembly 818 comprised of a negative portion 820 and a positive portion 822 that acts as a diode 824.

FIG. 20 illustrates a capacitative assembly 830 comprised of sheets 832 and 834 of beta-tubulin and, dispoed therebetween, polystyrene beads 836 on which are disposed kinesin proteins 838. As will be apparent, this assembly acts as a capacitor 840.

FIG. 21 illustrates an inductive assembly 850 that is comprised of a a microtubule 852 to which have been attached kinesin proteins 838. In one embodiment, magnetic antimitotic agent 860 is disposed within the core of microtubule 852.

In one embodiment, a switch 880 is constructed by connecting a recognition molecule 882 that binds to its recognition 884 only after it has been activated by the binding of cofactor 886, at which point current can flow from conductive fragment 888 to conductive fragment 890.

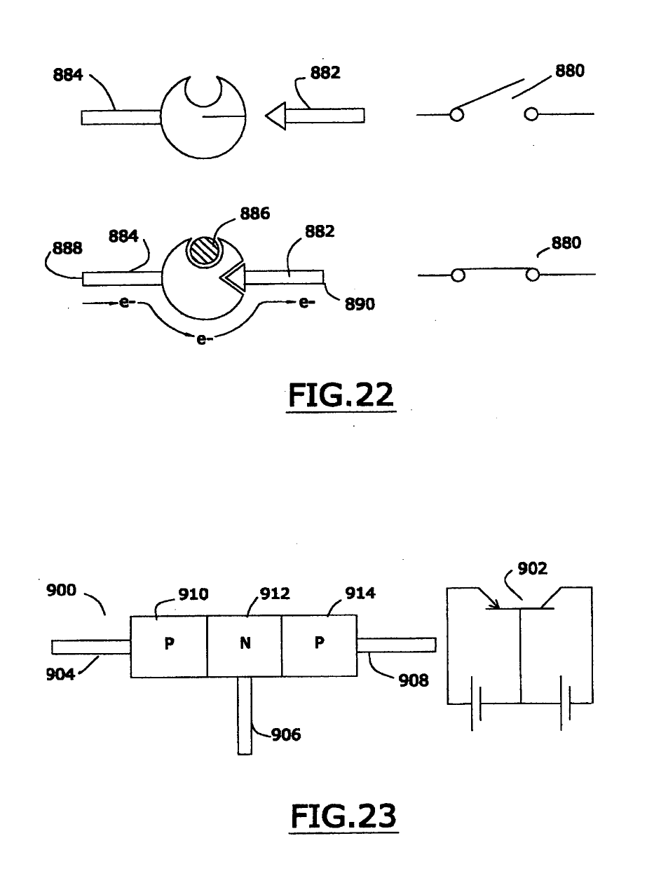

FIG. 22 illustrates an assembly 900 that whose equivalent circuit is 902. The assembly 902 is comprised of conductive links 904, 906, and 908 operatively connected to “P section 910,” “N section 912,” and “P section 914.”

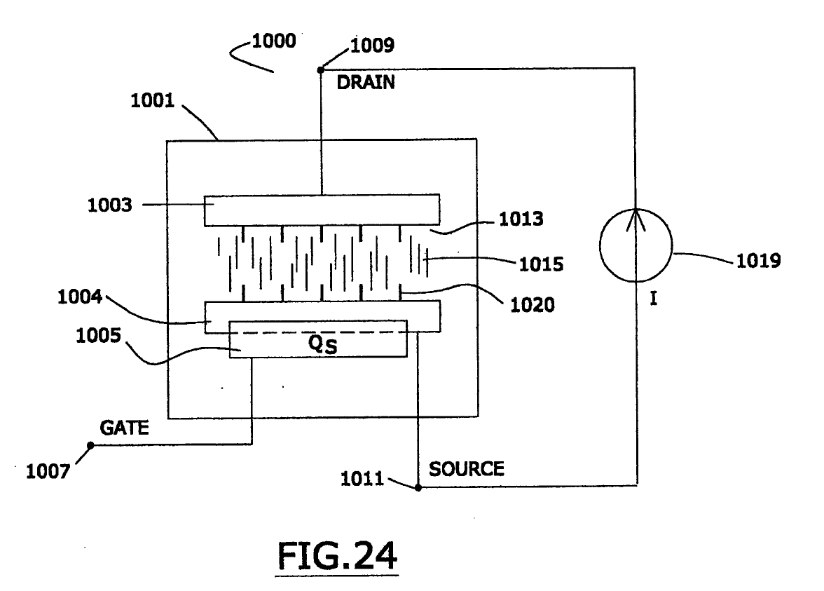

The ability to integrate traditional electronics with parts of the cell has great potential in the treatment of disease, delivery of drugs, communication and signaling between the cells of a living organism and an electronic device such as a computer, and in sensing and imaging the growth of unwanted cells such as cancer cells. However, there are few devices currently able to provide such integration. The microtubule based switching device as described herein uses a part of a eucaryotic cell called a microtubule to provide such integration. The microtubule is a part of a remarkable system of filaments within a cell called the cytoskeleton. A device that integrates traditional electronics with the cellular environment also opens the door for research and development into ways to control the growth and organization of cells, the ways in which cells organize and interact with their environment, change their shape and move from one location to another. Various embodiments of such a device are described by reference to FIGS. 24 through 30. In the drawings, like reference numerals have been used throughout to designate identical elements.

FIG. 24 is a plan view of a biological switching device according to one embodiment of the present invention. As used herein, the term biological switching device refers to a device comprised of biological material, the biological material preferably is cytoskeletal material, and that is also preferably comprised of a first electrical connection and a second electrical connection. Referring to FIG. 24, and to the preferred embodiment depicted therein, it will be seen that biological material 1015 is disposed between a microelectrode 1003 and a second microelectrode 1004. FIG. 24 depicts a charge coupled gate microtubule based switching device 1000. The charge coupled gate microtubule based switching device 1000 is based on biological material, and contains a charge source 1005, conductive microelectrodes 1003 and 1004, and a channel 1013 containing microtubules 1015. The charge coupled gate microtubule based switching device 1000 is a three terminal device that contains a drain 1009, a source 1011 and a gate 1007. The drain 1009 and the source 1011 refer to terminals of the charge coupled gate microtubule based switching device 1000 and indicate direction of current flow. Current flows from Drain 1009 to Source 1011. The drain 1009 is connected to a conductive microelectrode 1003. The source 1011 is also connected to a second conductive microelectrode 1004.

The conductive microelectrode 1003 and the second conductive microelectrode 1004 make up a microelectrode pair. The microelectrode pairs, in one embodiment, were created from a thin film layer of gold that, in some embodiments, may be pre-primed with a layer of titanium. The thin film layer of gold, in one embodiment, was placed on a thermally oxidized silicon wafer and the microelectrodes were formed using thin film deposition and photolithography techniques commonly known to those skilled in the art.

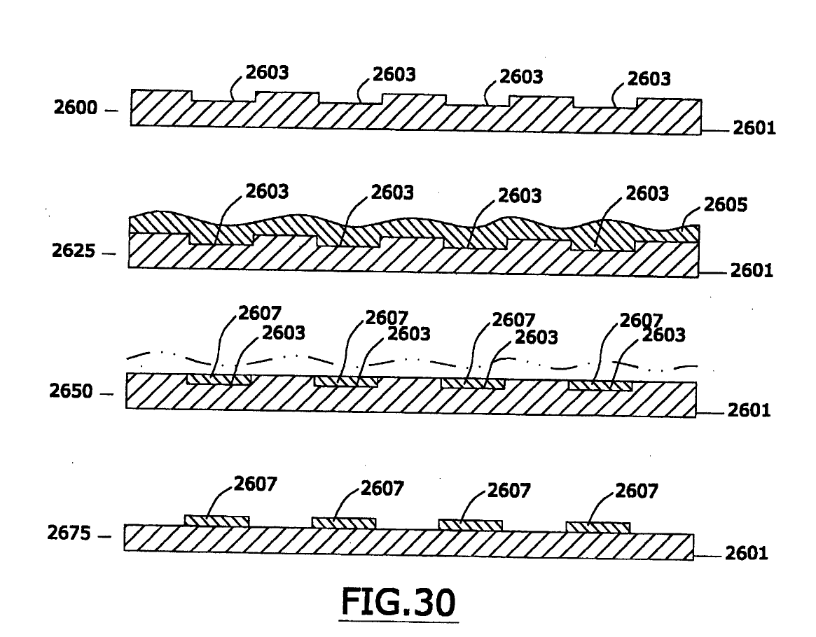

In some embodiments, nanoelectrodes 1020 are connected to the microelectrode 1003 and the second microelectrode 1004 to enable direct electrical contact between an individual microtubule 1015 and both the microelectrode 1003 and the second microelectrode 1004. In one embodiment, a plurality of nanoelectrodes 1020 extend into the channel 1013, forming a hair-like array. The nanoelectrodes 1020 increase the probability that the microtubules 1015 will make ohmic contact with the microelectrode 1003 and the second microelectrode 1004. The nanoelectrodes 1020 may be manufactured using a technique such as Electron Beam-induced Deposition (EBD). The nanoelectrodes 1020 may, in other embodiments, be manufactured using the process described in this specification and illustrated by way of FIG. 30. The nanoelectrodes 1020 may, in some embodiments, be fabricated using catalyst pattern techniques such as described in U.S. Pat. No. 6,831,017 entitled CATALYST PATTERNING FOR NANOWIRE DEVICES. Other methods may be used, for example, as described in U.S. Pat. No. 6,843,902 entitled METHODS FOR FABRICATING METAL NANOWIRES. The microelectrode 1003 and the second microelectrode 1004 form a channel 1013. The channel 1013 may be formed entirely from the geometries of the microelectrode 1003 and the second microelectrode 1004 in some embodiments. In other embodiments, the channel 1013 may be formed from a microelectrode 1003, a second microelectrode 1004 and additional geometries, for example, a well formed into a microstructured substrate such as silicon. A well is commonly known to one skilled in the art of microelectronics and microelectronic device design as an area depressed into a substrate such as silicon that may at times contain material that differs from the material of the surrounding substrate. A well is etched in, for example, a silicon substrate using proportions of HNO3, HF, CH3COOH and water. Other fabrication techniques may use anisotropic etching with etchants such as KOH and Hydrazine hydrate. In some embodiments, specific geometries of wells are formed by selective etching using resistive coatings to prevent the etching of the surrounding substrate. The depth of the well can be controlled by varying the strength of the etchant and the exposure time of the etchant to the substrate. In some embodiments, a sublayer of chrome silicon is sputter deposited to serve as an etch stop. The chrome silicon may, in some embodiments, be a ratio of 40% chrome and 60% silicon. The channel width for the purpose of this disclosure is defined as the spacing between the microelectrode 1003 and the second microelectrode 1004. The channel width may be varied to achieve the desired electrical characteristics at the drain 1009 and the source 1011. The drain 1009 and the source 1011 refer to terminals of the microtubule based switching device that indicate direction of current flow. Current flows from Drain 1009 to Source 1011. The channel 1013 may contain microtubules 1015 in a solution such as tubulin. The microtubules may, in some embodiments, be conductive. Conductive microtubules are known to those skilled in the art. As is disclosed in U.S. Pat. No. 6,452,564, the entire disclosure of which is hereby incorporated by reference into this specification, “ . . . these microtubules are preferably a system of biologically-derived, high-aspect ratio, rods or tubules of microscopic dimensions, and are made electrically conductive by electroless plating . . . .”

Preparation of conductive microtubules of U.S. Pat. No. 6,452,564 is described in the paragraph beginning at line 51 of column 4, wherein it is disclosed that: “The microtubules are based on research done a number of years ago, wherein researchers at the Naval Research Laboratories in Washington, D.C., discovered particles with the size and shape appropriate for percolation. These microtubules are biologically derived, hollow organic cylinders of half-micron diameter and lengths of tens to hundreds of microns. The cylinders are coated with metal to render them conductive by an electroless process. Once metallized, the microtubules can be dried to a powder and dispersed into polymer matrices at varying loading densities to form the composite. In a preferred embodiment, the microtubules are formed from diacetylenic lipid (1,2 bis(tricosa-10,12-diynoyl)-sn-glycero-3-phosphocholine), or DC8,9PC. See, for example, A. N. Lagarkov and A. K. Sarychev, Phys. Rev. B 53, 6318 (1996) and F. Behroozi, M. Orman, R. Reese, W. Stockton, J. Calvert, F. Rachfold and P. Schoen, J. Appl. Phys. 68, 3688 (1990). The lipid is dissolved in alcohol at 50° C., water is added, and the temperature lowered to room temperature. The lipid self-assembles itself into microtubules and subsequently precipitates. The particles are rinsed and coated with a palladium catalyst and mixed with metal ions and reductants. In contact with the catalyst, the metal ions-are reduced to neutral metal on the surface of the microtubules and coat the structure with a conductive layer of metal of several tenths of a micron thickness. Several metal species are available for use in this process, but nickel and copper appear to be of greatest potential usefulness for the present invention.”

The microtubules of U.S. Pat. No. 6,452,564 have a substantially uniform conductivity over their entire surface, such conductivity being substantially equal to the conductivity of the metal used to coat the surface.

The microtubules 1015 may, in some embodiments, be coated with a resistive material such as tantalum or nichrome (NiCr). In other embodiments, the microtubules may be coated with a magnetic material. The channel 1013 may, in some embodiments, contain several different types of microtubules. Microtubule types may, in some embodiments, include variations in both the biological and the electrical characteristics of microtubules.

It has been shown by Tuszynski, Hameroff et al in a paper entitled “Ferroelectric Behavior in Microtubule Dipole Lattices: Implications for Information Processing, Signaling and Assembly/Disassembly” that microtubules are charged dipoles, and will align in a parallel orientation upon the application of an electric field or a magnetic field.

Returning now to FIG. 24, a charge source 1005 is shown in proximity to the channel 1013. The charge source 1005 may be adjacent to the channel, or may be overlayed above or below the channel 1013. The charge source 1005 may in some embodiments be an electrode. The charge source in other embodiments may be a cathode. The charge source 1005 may be fabricated by deposition of a thin film layer of metal that, in some embodiments may be gold, and in other embodiments, may be pre-primed with a layer of titanium. The charge source geometry may be formed using thin film deposition and photolithography techniques commonly known to those skilled in the art. The charge source 1005 may, in some embodiments, be separated from the channel 1013 and, in some embodiments, the microelectrode 1003 and the second microelectrode 1004, with an insulator or dielectric. One preferred insulator is Silicon Dioxide. Other insulators may include Silicon Monoxide, ruby, or ceramic. Insulators such as silicon dioxide and silicon monoxide may be grown as an oxide layer on top of a silicon substrate using techniques known to those skilled in the art.

The charge source 1005 is connected to a gate 1007. The electrical function of the gate 1007 is similar to the gate of a metal oxide semiconductor field effect transistor (MOSFET). Upon application of a voltage, the charge source 1005 becomes electrically biased. The charge source 1005 in such an energized state will serve to either repel the negatively charged end of the microtubules 1015 contained in the channel 1013, or attract the negatively charged ends of the microtubules, depending on the bias that is applied to the gate 1007.

The alignment of microtubules 1015 in the presence of a charge is due to the inherent dipole moment of the microtubules 1015; whereby a positive charge applied to the gate 1007 causes the microtubules 1015 to align perpendicular to the surface of the microelectrode 1003 and the second microelectrode 1004, such that the negative end of the microtubules 1021 is oriented proximate to the charge source 1005. The alignment of the microtubules 1015 within the channel 1013 creates a conductive path between the microelectrode 1003 and the second microelectrode 1004. The magnitude of the applied charge determines the degree of alignment of the microtubules 1015. The greater the alignment of microtubules 1015, the lower the resistance of the channel 1013.

Changing the voltage bias at the gate 1007 will change the gain of the microtubule based switching device 1000 by altering the electrical characteristics such as microtubule alignment within the channel 1013.

The electrical connection 1007 at the charge source serves as the gate 1007 of the device. In a similar manner, the electrical connections 1009 and 1011 of the microelectrode 1003 and the second microelectrode 1004 serve as the drain and source of the device respectively. The electrical connections at the drain 1009, source 1011 and gate 1007 are made using wire bond techniques known to those skilled in the art of microelectronics fabrication. Wire bond techniques may include thermo compression bonding, ball bonding, nail head bonding, or ultrasonic binding. Referring again to FIG. 24, an ideal current source 1019 is shown. The ideal current source 1019 may be further connected to additional electronic components such as resistors (not shown) to provide voltage levels that are appropriate for the circuitry to which the microtubule based switching device 1000 is connected.

Referring now to FIG. 25, a cross sectional view of a microtubule based switching device 2000 according to another embodiment of the present invention is shown. The microtubule based switching device 2000 uses a gate 2007 that contains an infrared light source 2005. As will be further described, the infrared light source 2005 serves to align microtubules 2015 contained in a channel 2013. The infrared light source 2005 emits infrared light with a wavelength of from about 400 to about 900 nanometers. It has been shown that microtubules will align in a manner similar to that of a compass needle when exposed to Infrared light. Reference may be had to the article “Cell Intelligence” by Guenter Albrecht-Buehler. In this article, Buehler states that Centrioles are able to detect near Infrared Light, and cells use Centrioles to see objects around them that emit or scatter near infrared light.

Another article by Guenter Albrecht-Buehler, entitled “Rudimentary form of cellular ‘vision’” that was published in The Proceedings of the National Academy of Science of the United States of America, September 1992, Volume 89, pages 8288-829 discloses that cells located and tried to approach distant infrared light sources because they mistook them for other cells. This article further discloses that cells are continuously emitting and absorbing infrared light.

Referring again to FIG. 25, the infrared light source 2005 is shown in proximity to the channel 2013. The infrared light source 2005 may be adjacent to the channel, or may be overlayed above or below the channel 2013. The infrared light source 2005 may in some embodiments be mitochondria. In some embodiments, the infrared light source 2005 may be luminescent cells. Luminescent cells, as discussed by Dr. John W. Kimball in his textbook “Biology” (http://users.rcn.com/jkimball.ma.ultranet/BiologyPages/B/Bioluminescence.html) under the heading Bioluminescence are cells that emit light through the involvement of a luciferin, a light emitting substrate, a luciferase, an enzyme that catalyzes the reaction, ATP, the source of energy, and molecular oxygen, O2. Luminescent cells in fireflies, for example, contain nitric oxide synthase (NOS), the enzyme that liberates the gas nitric oxide (NO) from arginine. Nerve impulses activate the release of NO from these cells, the NO diffuses and inhibits cellular respiration in mitochondria by blocking the action of cytochrome c oxidase. With cellular respiration inhibited, the oxygen content of the cells increases, which turns on light production in the peroxisomes that contain luciferase and luciferin ATP. Luminescent cells may also, in some embodiments, be contained in luminescent bacteria such as those found in the flashlight fish, Photoblepharon palpebratus. The infrared light source 2005 in other embodiments may be a light emitting diode PN junction. In some embodiments, the infrared light source 2005 may be fabricated within the substrate 2001 using standard PN junction fabrication techniques commonly known to those skilled in the art of microelectronics fabrication. The infrared light source 2005 is connected to a gate 2007. Upon application of an external stimulus to the gate 2007, the infrared light source 2005 is activated. In some embodiments, the external stimulus is an applied voltage. The infrared light emitted by the light source 2005 serves to align the microtubules 2015 contained in the channel 2013. The microtubules 2015, in the presence of infrared light, align perpendicular to the surface of the microelectrode 2003 and the second microelectrode 2004. The alignment of the microtubules 2015 within the channel 2013 creates a conductive path between the microelectrode 2003 and the second microelectrode 2004. In some embodiments, nanoelectrodes 1020 are connected to the microelectrode 2003 and the second microelectrode 2004 to enable direct electrical contact between an individual microtubule 2015 and both the microelectrode 2003 and the second microelectrode 2004. In one embodiment, a plurality of nanoelectrodes 1020 extend into the channel 2013, forming a hair-like array. The nanoelectrodes 1020 increase the probability that the microtubules 1015 will make ohmic contact with the microelectrode 2003 and the second microelectrode 2004. The nanoelectrodes 1020 may be manufactured using a technique such as Electron Beam-induced Deposition (EBD). The nanoelectrodes 1020 may, in other embodiments, be manufactured using the process described by way of FIG. 30. The nanoelectrodes 1020 may, in some embodiments, be fabricated using catalyst pattern techniques such as described in U.S. Pat. No. 6,831,017 entitled CATALYST PATTERNING FOR NANOWIRE DEVICES. Other methods may be used, for example, as described in U.S. Pat. No. 6,843,902 entitled METHODS FOR FABRICATING METAL NANOWIRES.

Referring again to FIG. 25, an ideal current source 2019 is shown. The ideal current source 1019 may be further connected to additional electronic components such as resistors (not shown) to provide voltage levels that are appropriate for the circuitry to which the microtubule based switching device is connected.

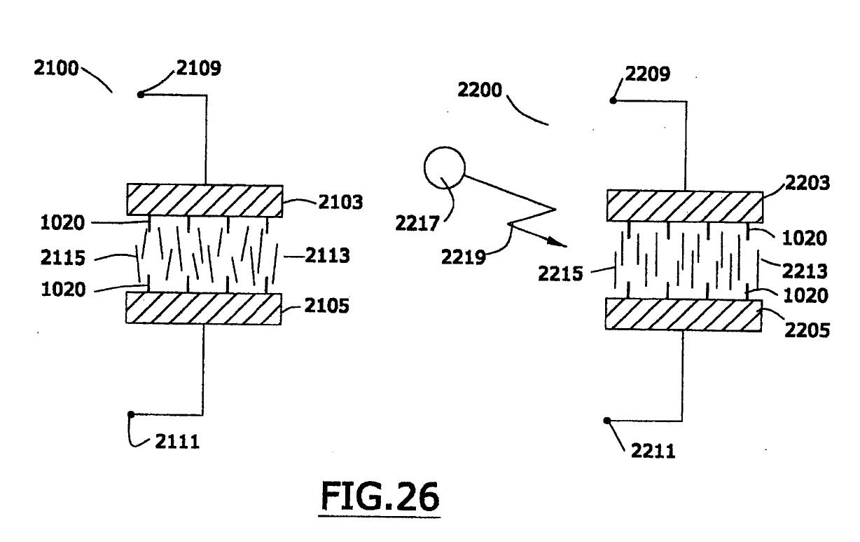

Referring now to FIG. 26, a time variant view of a biological switching device used as a sensor is shown. The microtubule based sensor 2100 and 2200 is a two terminal device similar to the three terminal microtubule based switching devices previously illustrated in FIGS. 24 and 25, but with the gate structure removed. A microtubule based sensor in the absence of infrared light 2100 exhibits random orientation of the microtubules 2115. A microtubule based sensor in the presence of infrared light 2200 exhibits orientation of the microtubules 2115 in a direction perpendicular to the microelectrode 2203 and the second microelectrode 2205.

The microtubule based sensor of FIG. 26 contains a channel 2113, a microelectrode 2103 and a second microelectrode 2105. The channel 2113 contains microtubules 2115. The microtubule based sensor shown in FIG. 26 is similar to the microtubule based switching device of FIG. 25 with the absence of a gate. The microtubule based sensor shown in FIG. 26 is a two terminal device that provides a conductive path upon detection of infrared light with a wavelength of from about 400 to about 900 nanometers, and a non-conductive or less conductive path in the absence of infrared light. The microtubules 2115 that are contained in the channel 2113 become aligned in the presence of infrared light. A microtubule based sensor in the absence of infrared light 2100 contains microtubules 2115 oriented randomly within a channel 2113. A microtubule based sensor in the presence of infrared light 2200 contains microtubules 2115 in a channel 2115 that align perpendicular to the surface of the microelectrode 2103 and the second conductive microelectrode 2105. The alignment of the microtubules 2115 within the channel 2113 creates a conductive path between the microelectrode 2103 and the second microelectrode 2105.

In some embodiments, nanoelectrodes 1020 are connected to the microelectrodes to enable direct electrical contact between an individual microtubule and the microelectrode 2103 or the second microelectrode 2105. The nanoelectrodes 1020 may be manufactured using a technique such as Electron Beam-induced Deposition (EBD). The nanoelectrodes 1020 may, in other embodiments, be manufactured using the process described by way of FIG. 30. The nanoelectrodes 1020 may, in some embodiments, be fabricated using catalyst pattern techniques such as described in U.S. Pat. No. 6,831,017 entitled CATALYST PATTERNING FOR NANOWIRE DEVICES. Other methods may be used, for example, as described in U.S. Pat. No. 6,843,902 entitled METHODS FOR FABRICATING METAL NANOWIRES. Other embodiments of the microtubule based sensor 2100 may use a surface acoustic wave structure as a structural part of the channel 2113. A surface acoustic wave structure is a microelectronic structure that uses a piezoelectric substrate with a thin film coating. The piezoelectric substrate may, in some embodiments, be quartz. Other embodiments may include Lithium Niobate (LiNbO3) or Lithium Tantalate (LiTaO3). The thin film coating may, in some embodiments, be polyisobutylene (PIB). Polyisobutylene is deposited to a thickness of between 5 and 30 microns using thin film deposition techniques such as sputter deposition, chemical vapor deposition, and the like. The thin film coating, in other embodiments, may be polyimide. Polyimide is deposited to a thickness of between 1 and 20 microns using thin film deposition techniques such as sputter deposition, chemical vapor deposition, and the like. The surface acoustic wave structure resonates upon exposure to RF energy, and returns a modified RF signal based on the material characteristics present in the channel 2113. The microtubule based sensor containing a surface acoustic wave structure as a part of the channel 2113 allows for interrogation of the sensor using an externally applied RF signal, and provides information on the state of the sensor as well as the state of the material contained in or adjacent to the channel 2113.

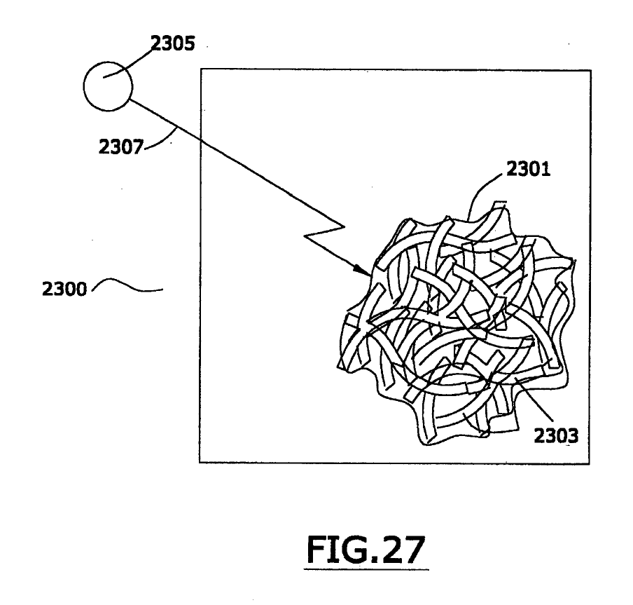

Referring now to FIG. 27, a biological memory array 2300 is shown. Microtubules 2303 are contained within an array 2301. The microtubules 2303 maintain spatial orientation based on addressing by an infrared light source 2305. The infrared light source 2305 emits infrared light 2307 that is optically modulated to address microtubules 2303.

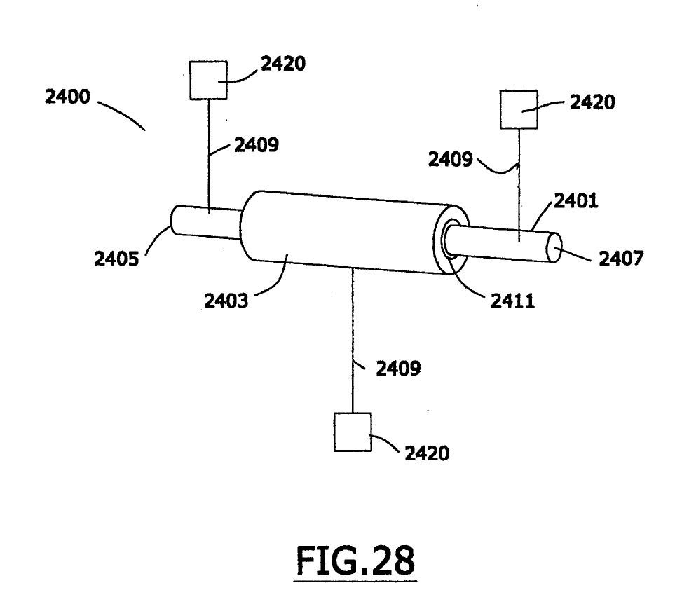

Referring to FIG. 28, a magnetic biological memory array element 2400 is shown. The magnetic biological memory array element 2400 comprises a microtubule 2401 with a magnetoresistive coating 2403. The magnetoresistive coating 2403 may, in some embodiments, be an anisotropic ferromagnetic thin film such as disclosed in U.S. Pat. No. 6,275,411 entitled SPIN DEPENDENT TUNNELING MEMORY. The magnetoresistive coating 2403 may, in a preferred embodiment, be a ferromagnetic thin film layer that is formed of an alloy of 65% nickel, 15% iron, and 20% cobalt that is deposited to a thickness of 40 angstroms, and which has a magnetic saturation of typically about 10,000 Gauss. In some preferred embodiments, the magnetoresistive coating 2403 may include a thin film layer of 5% iron and 95% cobalt having a thickness of 15 angstroms, resulting in a magnetic saturation induction of approximately 16,000 gauss. In some embodiments, multiple thin film layers may be separated by a barrier layer such as aluminum oxide. The microtubule 2401 contains a drain 2405 and a source 2407. The drain 2405 is electrically connected to a microelectrode 2420 using a nanowire 2409. The nanowire 2409 may, in some embodiments, be manufactured using the process described by way of FIG. 30. The nanowire 2409 may, in other embodiments, be fabricated using catalyst pattern techniques such as described in U.S. Pat. No. 6,831,017 entitled CATALYST PATTERNING FOR NANOWIRE DEVICES. Other methods may be used, for example, as described in U.S. Pat. No. 6,843,902 entitled METHODS FOR FABRICATING METAL NANOWIRES.

The source 2407 is electrically connected to a microelectrode 2420 using a nanowire 2409. The magnetoresistive material 2403 is further electrically connected to a bonding pad 2420 using nanowires 2409. In some embodiments, the magnetoresistive coating 2403 is separated from the microtubule 2401 with a dielectric layer 2411. The dielectric layer 2411 may be, for example, silicon nitride or silicon dioxide.

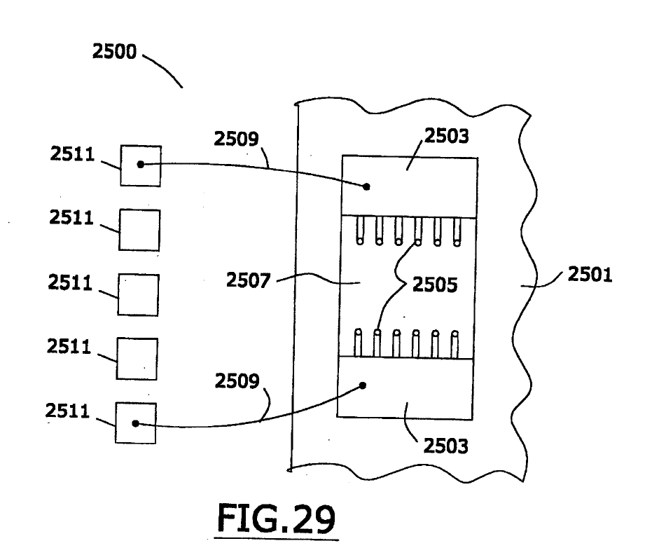

Referring now to FIG. 29, a plan view of a microelectrode and nanoelectrode structure 2500 according to one embodiment of the present invention is shown. A substrate 2501 may, in some embodiments, be used to mechanically retain the microelectrode and nanoelectrode structure 2500. Microelectrodes 2503 may be deposited using evaporation, sputtering, plating, anodization, chemical vapor deposition, and screen printing. Selective etching may be used to further remove excess metal from the microelectrode structure. The microelectrode 2503 may contain nanoelectrodes 2505. The nanoelectrodes may be manufactured using the process described later in this specification, and illustrated by reference to FIG. 30. The nanoelectrodes 1020 may, in some embodiments, be fabricated using catalyst pattern techniques such as described in U.S. Pat. No. 6,831,017 entitled CATALYST PATTERNING FOR NANOWIRE DEVICES. Other methods may be used, for example, as described in U.S. Pat. No. 6,843,902 entitled METHODS FOR FABRICATING METAL NANOWIRES. The nanoelectrodes 2505, in some embodiments, project into a channel 2507. The channel 2507 may, in some embodiments, contain a well formed into a microstructured substrate such as silicon. A well is commonly known to one skilled in the art of microelectronics and microelectronic device design as an area depressed into a substrate such as silicon that may at times contain material that differs from the material of the surrounding substrate. A well is etched in, for example, a silicon substrate using proportions of HNO3, HF, CH3COOH and water. Other fabrication techniques may use anisotropic etching with etchants such as KOH and Hydrazine hydrate. Specific geometries of wells are formed by selective etching using resistive coatings to prevent the etching of the surrounding substrate. The depth of the well can be controlled by varying the strength of the etchant and the exposure time of the etchant to the substrate. In some embodiments, a sublayer of chrome silicon is sputter deposited to serve as an etch stop. The chrome silicon may, in some embodiments, be a ratio of 40% chrome and 60% silicon. The microelectrodes 2503 are electrically connected to a bonding pad 2511 using wire bonding techniques such as thermocompression, ball or nail head wire bonding. In some embodiments, ultrasonic bonding may be used. The bonding pad 2511 may, in some embodiments, be contained in a chip carrier (not shown).

Referring now to FIG. 30, a fabrication method for manufacturing nanowires is shown. 2600 is a substrate 2601 etched with wells 2603. The wells are etched in, for example, a silicon substrate using proportions of HNO3, HF, CH3COOH and water. Other embodiments may use anisotropic etching with etchants such as KOH and Hydrazine hydrate. The substrate 2601 is selectively etched using resistive coatings. The depth of the well can be controlled by varying the strength of the etchant and the exposure time of the etchant to the substrate. In some embodiments, a sublayer of chrome silicon is sputter deposited to serve as an etch stop. The chrome silicon may, in some embodiments, be a ratio of 40% chrome and 60% silicon. 2625 shows a substrate 2601 containing wells 2603 that have been coated with a thin film metal using a method such as magnetron sputter deposition, RF sputter deposition, chemical vapor deposition, or the like. 2650 shows the same substrate 2601 with the excess metal etched away to a expose the substrate 2601. The wells 2603 are now filled with a metal 2607 such as gold, aluminum, silver, copper, platinum, or the like. 2675 now shows the same substrate 2601 with further etching. The metal 2607 is now exposed above the surface of the substrate 2601, making nanowires.

Modeling and Prediction of Microtubule Dynamics

As is well known to those familiar with their properties, microtubules are in a constant cycle of assembly and disassembly. The relative stability of an individual microtubule's length and the amount of time said microtubule remains assembled is determined by a multitude of cellular conditions which include but are not limited to: concentration of GTP and/or GDP, concentration of tubulin monomers, temperature, pH, concentrations of cytoplasmic salts, and other factors. Determination of the stability and or rate of growth and/or collapse of microtubules relative to time and predictions of these features allow for a powerful method to interrogate, among other things, cell health, position in the cell cycle, timing of cell division and control of cellular processes.

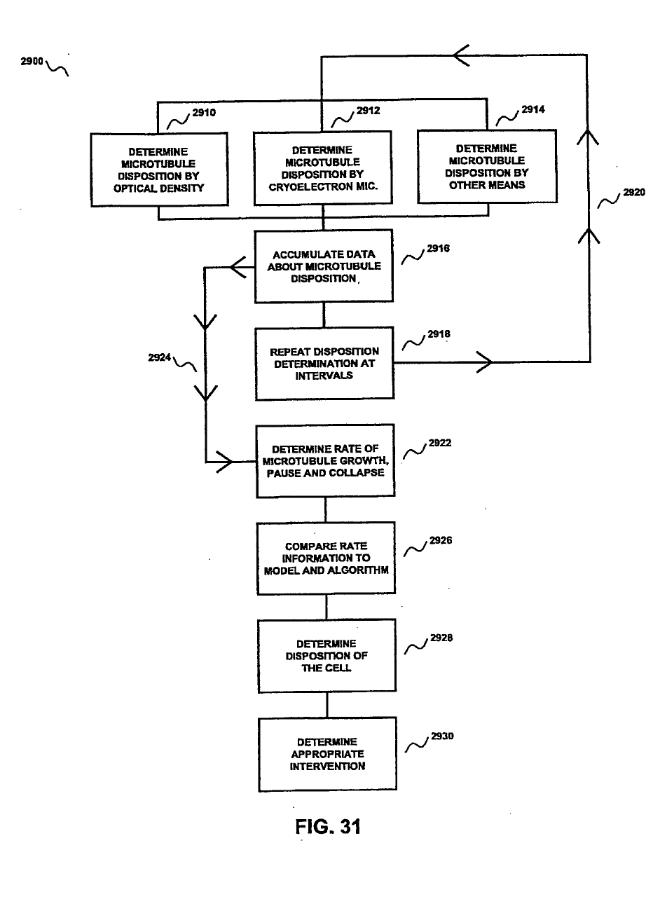

FIG. 31 is a flow diagram that describes a method 2900 for acquiring data about the disposition of a cell's microtubules, for applying these data to a mathematical algorithm, and for relating this processed information to cell health, position in the cell division cycle, or other experimentally determined disposition.

Steps 2910, 2912 and 2914 of FIG. 33 involve the determination of the length of microtubules. In one preferred embodiment, the microtubules involved in these steps are derived from a cell monolayer preferably grown in a laboratory incubator. As is known to those skilled in the art, the microtubules' length is often referred to as its “disposition.”

Referring again to FIG. 31, the microtubule disposition may either be determined by conventional optical density (using a spectrophotometer measuring absorbance of visible light at a wavelength of either 280 or 340 nanometers), as is described in step 2910. Alternatively, or additionally, the microtubule disposition may be determined by cryelectron microscopy, and/or by other conventional means. Reference may be had, e.g., to U.S. Pat. No. 4,857,735 (light emitting diode spectrophotometer), U.S. Pat. No. 5,184,193 (dual fiber optic spectrophotometer), U.S. Pat. No. 5,413,098 (path constrained spectrophotometer), U.S. Pat. No. 6,654,119 (scanning spectrophotometer for high throughput fluorescence detection), U.S. Pat. No. 6,813,024 (non-focusing optics spectrophotometer), and the like. The entire disclosure of each of these United States patents is hereby incorporated by reference into this specification.

Referring again to FIG. 31, in step 2916, data is accumulated regarding the disposition of the microtubles, preferably by a machine such as a computer (not shown) which is capable of accumulating information acquired from steps 2910, 2912 and/or 2914 in the form of a database or hardcopy, or other recording method.

In the preferred embodiment illustrated in FIG. 31, and in step 2918 thereof, the length of the microbules is repeatedly measured at different time by means such as, e.g., a computer (not shown) and/or a computer program (not shown) and/or a relay switch (not shown). As will be apparent to those skilled in the art, one may use many other conventional means for repeatedly measuring the disposition of the microtubules. As is also known to those skilled in the art, the product of the length and number of microtubules is proportional to the optical density measured by, e.g., one or more of the aforementioned spectrophotometers.

In step 2922, one determines the rate of microtubule gowth, pause, and collapse, preferably by means of an algorithm (not shown) which is preferably, but not exclusively, a computer program that acquires information in step 2916 by way of connection of 2924. One may determine the rate of microtubule growth, pause, and collapse by conventional means and/or by means described elsewhere in this specification.

In the preferred embodiment depicted, item 2924 is a connective element (such as, e.g., a computer cable); and it may also comprise a a computer program or other suitable interface between the interpretive apparatus used in step 2922 and the stored information obtained in step 2916. In Step 2922, one preferably determines, based on the length of the microtubules in the sample versus time, the stability of the microtubules.

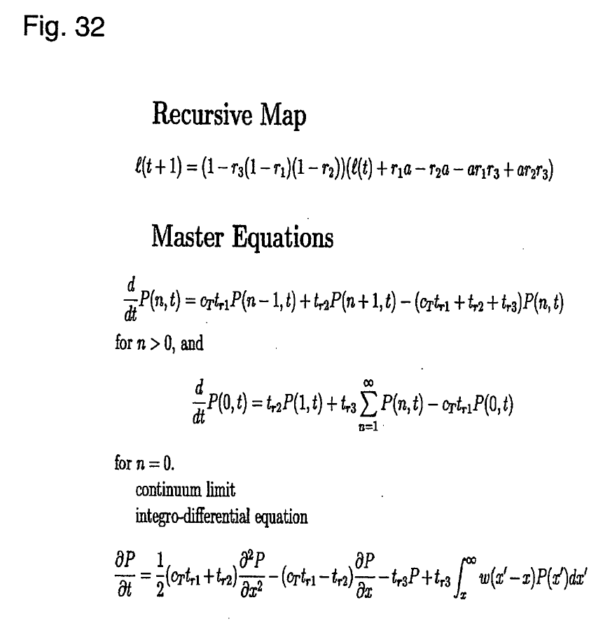

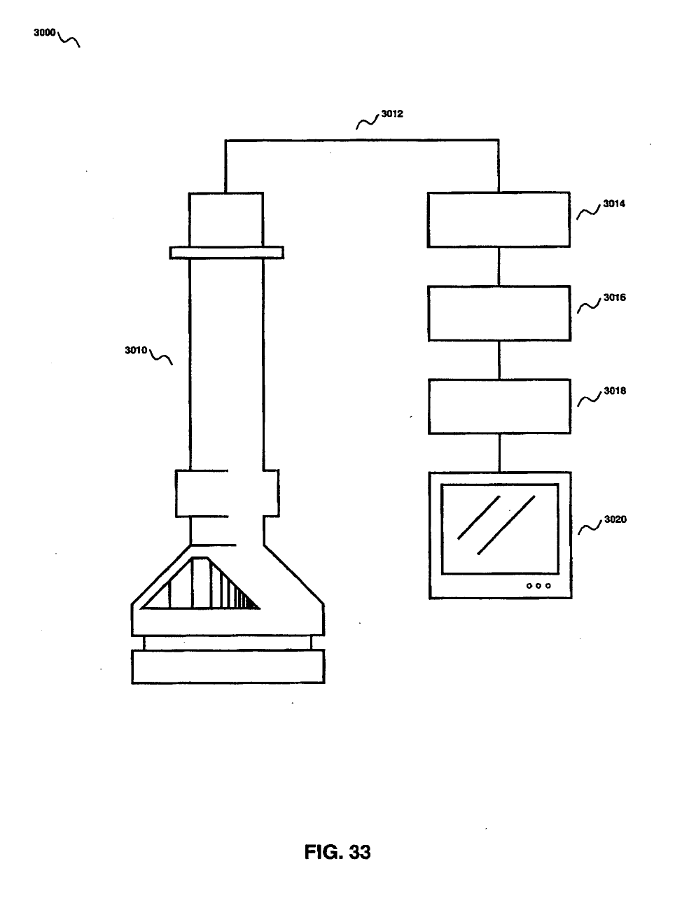

FIG. 32 illustrates some equations that may be used to model the stability, instability, or catastrophic disassembly of microtubules. One may use data from the steps described in FIG. 31 in the euquations depicted in FIG. 32.

Referring to FIG. 32, the “Recursive Map” provides a prediction based upon the length of an individual microtubule and determines its stability, rate of disassembly, and/or its possibility of catastrophic collapse. In the equation presented, 1 is the length of the microtubule, t is the time, r is a number (either 1 or 0) that represents a state of assembly or disassembly of the microtubule in question.

Referring again to FIG. 32, the “Master Equations” predict the continuous rate of growth and collapse of the microtubules, allowing for the construction of a histogram that can be plotted with length on the y axis and length at time t on the x axis. The slope of the three resulting lines represent the probability that a microtubule will stay the same length, or will go into catastrophic collapse. Correlation of such slope with the slopes of reference cells will indicate whether the particular cells in question are healthy or diseased; and such correlation also allows one to predict the position of the cells within the cell division cycle. As will be apparent to those skilled in the art, the “Master Equation” provides the time evolution of the probability distribution of microtubule lengths.

Referring again to FIG. 31, and in step 2926 thereof, one can compare the rate information obtained in step 2922 to the model and algorithm obtained via the equations of FIG. 32. In one embodiment, a flow chart showing possible outcomes with different values of r and in different sequences can be constructed that correlates rates of growth, pause and collapse with physiologic states of the cell. In one aspect of this embodiment, plotting the delta in length a between time n and n+1 in Step 2928 yields three discrete linear plots whose slopes indicate three possible outcomes: growth, shrinkage, or catastrophe. The slope of each of these curves can be used to determine the isotype of tubulin present in the cell which can be used to predict the tumorigenicity of the cell. Physicians, pathologists and clinicians determine appropriate intervention, if required, in step 2930.

Referring again to FIG. 31, and in step 2912 thereof, a cryoelectron microscope is preferably used to determine the disposition of the microtubules. As is known to those skilled in the art, a cryoelectron microscope contains means for “snap freezing” a biological sample so that the individual components of a cell can be observed at the micrometer level of detail without the use of stains, fixatives, or other invasive means. Reference may be had, e.g., to European Patent 1209469A1 which discloses that “A suspension of AVPs was applied to a holey carbon-foil grid and vitrified by flash-freezing in liquid ethane. The grids were cryo-transferred to the liquid nitrogen-cooled cryoelectron microscope (Philips CM200 FEG, FEI GmbH, Germany). Images were taken at a magnification of 60 000 under liquid nitrogen conditions at 1.5 microns defocus at 160 keV.” Reference also may be had, e.g., to U.S. Pat. Nos. 6,271,592 and 6,835,395, the entire disclosure of each of which is hereby incorporated by reference in to this specification.

In one embodiment illustrated in FIG. 33, a microscope 3000 is illustrated that can be used to look at the disposition of the microtubules in a cell sample. Microscope 3000 is comprised of an electron source and sample stage (see element 3010) collects information from a cell monolayer that has been quickly frozen with liquid helium. The information collected in element 3010 is communicated by element 3012 to a signal amplifier, or similar device shown as item 3014. Interpretation of the information by a computer, or other CPU containing device in 3016 allows element 3018, a device containing the capability to perform the algorithms described above in FIG. 32; and it also allows for a technician to see the result of the work on a display 3020, or other similar device.

A Method for Repair of Nerve and Spinal Cord Damage

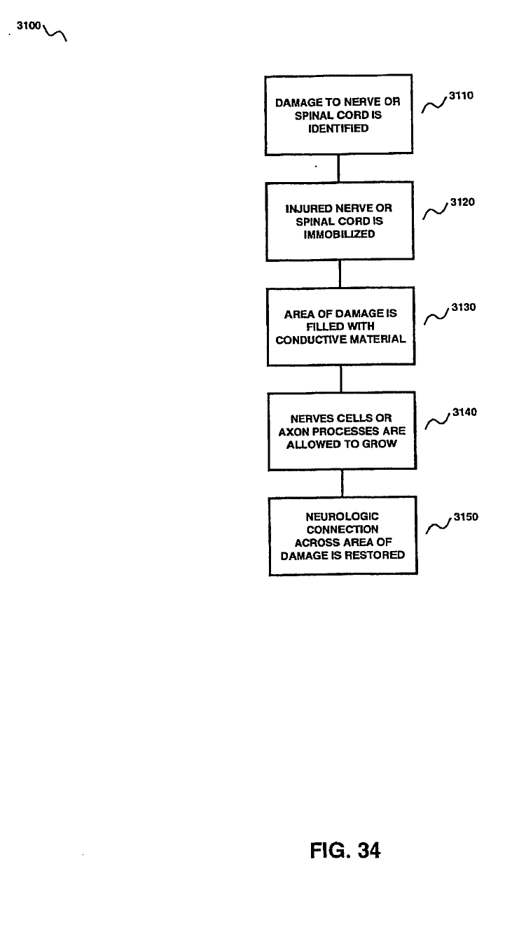

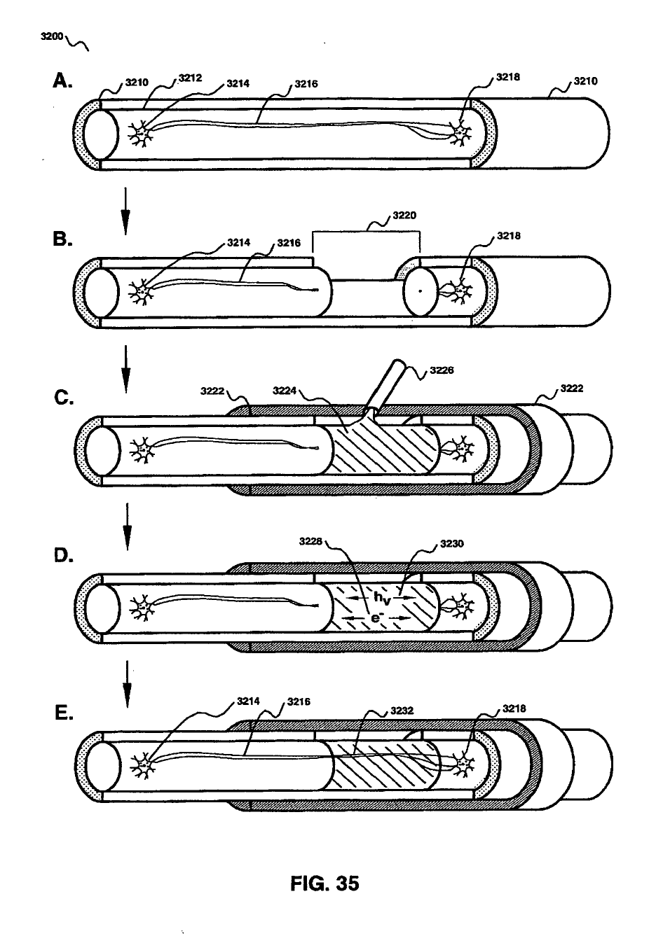

FIGS. 34 and 35 describe methods 3100 and 3200, respectively, for the repair of nerve damage produced by injury or disease, in the peripheral or central nervous system. In these embodiments, one may repair nerve damage by filing a gap with a solution of chemical and biological substrates that conduct electrical and/or light energy and/or magnetic energy.

Referring to FIG. 35, and in step 3110 thereof, damage to the nerve or the spinal cord of a biological organism is identified. Such damage may be, e.g., the gap 3220 illustrated in FIG. 35B.

Such damage is also described, e.g., in U.S. Pat. No. 6,676,675, the entire disclosure of which is hereby incorporated by reference into this specification. As is disclosed in such U.S. Pat. No. 6,676,675 (see columns 1-2), “When a nerve is severed, a gap is formed between the proximal and distal portions of the injured nerve. In order for the nerve axon to regenerate and reestablish nerve function, it must navigate and bridge the gap. Under the appropriate conditions, e.g., minimal gap length, the proximal end forms neurite growth cones that navigate the gap and enter endoneural tubes on the distal portion. The growth cones sense the extracellular environment and determine the rate and direction of nerve growth. The motion of the axon is responsive to environmental signals provided by other cells that guide the growth cone (Tessier-Lavigne, 1994).”

U.S. Pat. No. 6,676,675 also disclose that “Once the growth cones reach the distal segment, they enter the endoneurial tubes left from the degenerated axons. However, the growth cones and the dendrites on the proximal stump typically grow in many directions and unless the injury is small, the growth cones may never reach the distal segment. The natural ability of the nerve to regenerate is greatly reduced by the disruption of environmental cues resulting from, for example, soft tissue damage, formation of scar tissue, and disruption of the blood supply (Mackinnon and Dellon, 1988; Fawcett and Keynes, 1990, Buettner et al, 1994).”

U.S. Pat. No. 6,676,675 also disclose that “Several techniques have previously been attempted to aid and accelerate the repair of damaged nerves: suturing the severed ends, suturing an allograft or autograft, or regenerating the nerve through a biological or synthetic conduit (Williams et al., 1983; Valentini et al., 1987; Aebischer et al., 1988; Feneley et al., 1991; Calder and Green, 1995).”

U.S. Pat. No. 6,676,675 also disclose that “Autografts and allografts require a segment of a donor nerve acquired from the patient (autograft) or a donor (allograft). The donor nerve segment is removed from another part of the body or a donor and then sutured between the unattached ends at the injury site. Nerve autograft procedures are difficult, expensive, and offer limited success. Often, a second surgical procedure is required and may lead to permanent denervation at the nerve donor site. Allografts typically require the use of immunosuppressive drugs to avoid rejection of donor segments.”

U.S. Pat. No. 6,676,675 also disclose that “Artificial nerve grafts have been used in attempts to avoid the problems associated with autografts and allografts. Artificial grafts do not require nerve tissue from another part of the body or a donor. However, use of artificial nerve grafts has met with only limited success. Axonal regeneration in the peripheral nervous system has only been achieved for graft lengths up to approximately 3 cm in nonhuman primates. There has been little or no success with longer grafts. The previously used artificial nerve grafts were unsuitable for bridging longer gaps between distal and proximal nerve stumps and, therefore, are unsuitable for treating a significant proportion of nerve injuries.”

U.S. Pat. No. 6,676,675 also disclose that “Neurite growth has been aided to a limited extent by fabricating grooves on substrate surfaces (Weiss, 1945; Turner, 1983; Clark et al., 1987; Dow et al., 1987). The grooves employed in these studies were engraved on plastic or quartz substrates. The substrates utilized are unsuitable for implantation in vivo and thus the authors were unable to determine if the grooves could guide neurite growth in an animal. Alignment of neurons using physical guidance cues alone is highly uncertain and difficult to reproduce. For example, the neurites are typically aligned on only part of the substrate and unaligned on other parts and exhibit undesireable axon branching.”

U.S. Pat. No. 6,676,675 also disclose that “Tai et al., 1998 refer to the effects of micropatterned laminin glass surfaces on neurite outgrowth and growth cone morphology. In Tai et al., micropatterns consisting of laminin stripes alternating with glass stripes were formed on glass coverslips. Neuronal cultures were prepared from chicken dorsal root ganglia and seeded on either micropatterned laminin coverslips or on a uniform laminin coated glass surface. While neuronal growth on the micropatterned laminin surface was biased in the direction of the pattern, severe axon branching formed dense axon outgrowth. Thus, while the laminin provided some level of chemical guidance, applicability of the technique was limited. In addition, the glass substrates are unsuitable for implantation into patients.”

U.S. Pat. No. 6,676,675 also disclose that “Biodegradable conduits filled with magnetically aligned collagen rods have also been used in an attempt to provide directional guidance to regenerating neurons. However, this approach does not provide any chemical guidance to regenerating neurons and has had only limited success. The presence of the collagen rods reduces the space available for neuronal outgrowth, constricts growth, does not reduce axonal branching, and limits the natural transport of oxygen, nutrients, and waste products.” In one embodiment, and referring again to step 3110 of FIG. 34, steps may be taken by a physician in charge of an individual patient's well being following and injury or the diagnosis of neurodegerative disease.

Though not wanting to be limited to spinal injury as a sole source of this inventions therapeutic value, a normal healthy spinal cord is shown for the sake of illustration, in schematic form and cross-section, in FIG. 35 in letter A of item 3200. The bony and cartilaginous tissue is shown here as item 3210. Two healthy neurons (3214 and 3218) reside in full health in the spinal cord, 3212, connected by axon process 3216.

Following an injury or degenerative process, illustrated here in letter B of item 3200, a gap is caused to form in the cord 3212, separating the nerve cells 3214 and 2318.

The first requirement is an intervention to immobilize the damaged cord and prevent movement of the area, which could cause further damage. This can be accomplished, but is not restricted to, a casting material of metal or other material, as shown in letter C of item 3200, labeled 3222.

Referring again to FIG. 34, and in step 3120 thereof, the injred nerve or spinal cord is immobilized by conventional means. Reference may be had, e.g., to FIG. 35C; see the use of the immobilizing device 3222.

In step 3130 of FIG. 34, the damaged area in the nerve or spinal cord is filled with a biological compatibile conductive material. Thus, by way of illustration and not limitation, one may use conductive polymer gels. Reference may be had, e.g., to U.S. Pat. No. 6,434,410 (liquid electrolytic gel with a high salt concentration), U.S. Pat. No. 6,482,299 (polymer gel electrode), and the like; the entire disclosure of each of these United States patents is hereby incorporated by reference into this specicification. Reference also may be had to U.S. published patent application 20030074042 (differential gel body for a medical stimulation electrode), the entire disclosure of which is hereby incorporated by reference into this specification.

Referring to FIG. 35, and in the preferred embodiment depicted therein, it is preferred that the gel material 3224 preferably have an electrical conductivity that is from about 0.1 to about 10 times as great as the conductivity of neurons and, more preferably, is from about 0.5 to about 5 times the conductivity of neurons. In one preferred embodiment, the gel material 3224 has a resistivity.

In one embodiment, the gel material 3224 is transparent to light with a wavelength of from 100 to about 1200 nanometers and, more preferably, from about 200 to about 800 nanometers. As used herein, the term transparent refers to material that has a transmittance to such light of at least 80 percent. As is known to those skilled in the art, transmittance is the ratio of the radiant power transmitted by an article (i.e., the gel material 3224) to the incident radiant power.

In one preferred embodiment, the gel material 3224 is comprised of at least about 1 percent of microtubules, weight/volume, and, more preferably, at least about 5 percent of microtubules, weight/volume. In one aspect of this embodiment, the gel material 3224 contains from about 5 to about 20 percent of microtubules, weight/volume.

In one preferred embodiment, the gel material 3224 contains at least about 1 weight percent of unpolymerized (monomeric) tubulin and, more preferably, from about 1 to about 20 weight percent of unpolymerized tubulin. The unpolymerized tubulin is preferably selected from the group consisting of alpha-tubulin, beta-tubulin, gamma-tubulin, and mixtures thereof.

In one preferred embodiment, the gel material 3224 is comprised of at least about 1 percent of actin, weight/volume, and, more preferably, at least about 5 percent of actin, weight/volume. In one aspect of this embodiment, the gel material 3224 contains from about 5 to about 20 percent of actin, weight/volume.

In one preferred embodiment, the gel material 3224 contains at least about 1 weight percent of unpolymerized (monomeric) actin and, more preferably, from about 1 to about 20 weight percent of unpolymerized actin.

In one embodiment, in addition to one or more of the aforementioned materials, the material 3224 may contain one or more of calcium salt, sodium salt, magnesium salt, phospholipids, ion chelating agent (such as, e.g., EDTA, EGTA, and the like), energy sources (such as adenosine triphosphate, adenosine diphosphate, NADPH, carbohydrate such as glucose), peptide growth factor (such as EGF, TGF beta, VEGF, cytokines, and the like), antibiotic agents (such as ampicillin, tetracycline, streptomycin, and the like), gelling agent (such as agarose, acrylamide, complex carbohydrate, and the like), and the like. Other suitable agents will be apparent to those skilled in the art.

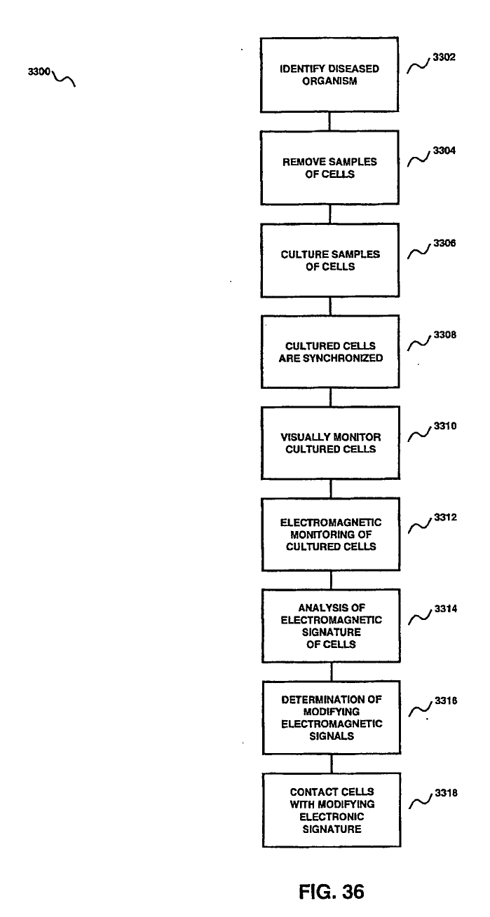

Referring again to FIG. 35, the gap 3220 (see FIG. 35B) may be filled by conventional means such as, e.g., with a tubue or syringe 3226 (see FIG. 35C). As is shown in step 3130 of FIG. 35, a tube or syringe, labeled 3226 in letter C of FIG. 36, can be used to fill the gap in the spinal cord or nerve with the conductive biological gel material, labeled 3224 in FIG. 36.

Referring again to FIG. 34, and in step 3140, the nerve cells or axon processes are allowed to grow through them aterial 3224, as is illustrated in FIGS. 35D and 35E of FIG. 35. Without wishing to be bound to any particular theory, it is believed that the process shown in letter FIG. 35D involves the transmission of electrical and light energy through material 3224, thereby allowing cells 3214 and 3218, on either side of the gap 3220 (see FIG. 35B) to communicate and grow in the direction of each other. This growth, as is illustrated in Figure FIG. 35E (also see steps 3240 and 3150 of FIG. 34) allow for reformation of synaptic connections (see FIG. 35E and the intact neuronal connection 3232 depicted therein). Such an intact connection 3232 faciliates restoration of cell communication and, for the organism in question, the return of function and sensation.

As will be apparent to those skilled in the art, the processes depicted in FIGS. 34 and 35 are applicable only to spinal cause injuries but, e.g., can be ued to repair detached retinas or other nerve-bundle maladies.

A Device for Altering the Electromagnetic Environment within a Biological Organism

It is known that the cells of biological organisms are capable of detecting infrared radiation with a wavelength of from about 400 to about 900 nanometers. This phenomenon was reported in an article by Guenter Albrecht-Buehler, entitled “Rudimentary form of cellular ‘vision,’” and published in Proceedings of the National Academy of Science of the United States of America, Volume 89, pages 8288-8292, September, 1992. In the first paragraph of this article, it was disclosed that “A previous article had suggested among other possibilities that 3T3 cells located and tried to approach distant infrared light sources because they mistook them for other cells (1).” The reference “(1) cited in the 1992 article was to a 1991 publication by Guenter Albrecht-Buehler published in the Journal of Cell Biology, 114, a pages 493 to 502.

In the abstract of the 1992 Guenter Albrecht-Buehler article, it was stated that “BHK cells were inoculated sparsely on one face of a thin glass film whose opposiste face was covered with a 2- to 3-day old confluent layer of BHK cells . . . . After 7 hr of attaching and spreading in the absence of visible light, most of the clls on the s-face traversed with their long axes the direction of the whorls of the confluent cells on the c-face directly opposed. The effect was inhibited by a thin metal coating of the glass films. The results suggest that the cells were able to detect the orientation of others by signals that penetrated glas but not thin metallic films and, therefore, appeared to be carried by electromagnetic radiation. In contrast, the effect was not influenced by a thin coat of silicone on the glass, suggesting that the wavelength of this radiation is likely to be in the red to infrared range. The ability of cells to detect the direction of others by electromagnetic singals points to a rudimentary form of cellular ‘vision.’”

At page 8292 of the 1992 Guenter Albrecht-Buehler article, it is disclosed that cells are continuously emitting and absorbing infrared light. The author states that “ . . . cells and all other objects in their environment at 37 degrees K interact continuously with the natural heat radiation of 310 K. Correspondingly, they are continuously emitting and absorbing infrared light over a wide range of wavelengths with a peak at about 10 microns (4).” The cited reference “(4)” was to a work by R. A. Smith et al. entitled “The Detection and Measurement of Infrared radiation” (Clarendon, Oxford, 1957).

As is well known to those skilled in the art, and as is disclosed, e.g., by the Smith work, means for measuring the infrared radiation produced by “ . . . cells and all other objects in their environment . . . ” (and other electromagnetic radiation) are well known. Reference also may be had, e.g. to U.S. Pat. No. 3,568,662 (apparatus for sensing bioelectric potentials), U.S. Pat. No. 3,557,777 (magnetic study of bioelectric phenomena), U.S. Pat. No. 3,662,746 (apparatus for detecting, analzing, and recording bioelectric potentials), U.S. Pat. No. 3,795,241 (electrode for recording biological properties), U.S. Pat. No. 3,880,146 (noise compensation techniques for biolelectric potential sensing), U.S. Pat. No. 3,971,365 (bioelectrical impedance measurement system), U.S. Pat. No. 4,275,743 (measuring device for measuring the bioelectrical activity of the central nervous system), U.S. Pat. No. 4,375,219 (electrode for detecting bioelectrical signals), U.S. Pat. No. 4,448,199 (electrode for detecting bioelectrical signals), U.S. Pat. No. 4,880,014 (method for determining therapeutic drug dosage using bioelectrical resistance and reactance measurements), U.S. Pat. No. 4,919,143 (electroencphalic neurofeedback apparatus and method for bioelectrical frequency inhibition and facilitation), U.S. Pat. No. 4,940,060 (apparatus for detecting bioelectric signals), U.S. Pat. No. 4,974,602 (arrangement for analyzing local bioelectric currents in biological tissue complxes), U.S. Pat. No. 5,024,227 (bioelectrical electrode), U.S. Pat. No. 5,024,235 (electroencephalic neurofeedback apparatus and method for bioelectrical frequtency inhibition and facilitation), U.S. Pat. No. 5,086,781 (bioelectric apparatus for monitoring body fluid compartments), U.S. Pat. No. 5,203,344 (method for taking bioelectrical impedance measurements), U.S. Pat. No. 5,307,817 (biotelemetry method for the transmission of bioelectrical potential differences), U.S. Pat. No. 5,464,014 (display device for bioelectrical and biophysical phenomena), U.S. Pat. No. 5,483,967 (bioelectric signal recording device), U.S. Pat. No. 5,795,293 (reducing artifact in bioelectrical signal modeling), U.S. Pat. No. 6,138,044 (method and device for sensing bioielectrical signals), U.S. Pat. No. 6,295,468 (apparatus for measuring bioelectrical parameters), and the like. The entire disclosure of each of these United States patents is hereby incorporated by reference into this specification.

In one preferred embodiment, the device used for measuring the infrared radiation produced by “ . . . cells and all other objects in their environment . . . ” is especially adapted to measure radiation in the near infrared range, from about 750 nanometers to about 3 microns. Such devices are also well known. Reference may be had, e.g., to U.S. Pat. No. 4,692,620 (near infrared measuring instrument with sample holder), the entire disclosure of which is hereby incorporated by reference into this specification.

FIG. 36 is a flow diagram of a preferred process 3300 for altering the electromagnetic environment of a biological system or a portion thereof. Referring to FIG. 36, and in tsep 3302 thereof, a diseased organism that is to be treated by the process in question is identified. The diseased organism may, e.g., be a human being suffering from cancer and/or another malady.

In step 3304, a sample of cells are removed from the diseased organism. Preferably this sample will include both healthy cells and cells that are affected by the malady in question. Thus, e.g., with a patient with cancer, one may by a biopsy remove both cancerous and noncancerous cells.

In step 3306, the healthy and unhealthy cells are separately cultured to produce a substantial number of cultured cells for evaluation. These cells thereafter as measured with one or more of the measuring instruments described hereinabove to determine what electromagnetic radiations they normally emit.

In step 3308, the cultured cells are synchronized in order to achieve synchronous growth. As is known to those skilled in the art, synchronous growth is growth in which all cells are at the same stage in cell division at any particular time. Such synchronous growth may be achieved by well known means. Reference may be had, e.g., to U.S. Pat. No. 5,158,887 (process for massive conversion of clostridia in synchronized cells of elongated length or refractive endospheres), U.S. Pat. No. 6,767,734 (method for producing age-synchronized cells), and the like. The entire disclosure of each of these United States patents is hereby incorporated by reference into this specification.

In step 3310, the cultured and synchronized cells are visually monitored to determine that stage(s) they are undergoing in the cell division cycle, and also to determine when cell synchronization is complete. Thus, step 3310 may advantageously occur prior, during, and after the cell synchronization process.

In step 3312, the synchronized and cultured cell samples are continually monitored to determine what electromagnetic radiations(s) each of such samples emits at various periods of time. During this monitoring, they are maintained at ambient temperature (37 degrees Fahrenhit), ambient pressure, and in standard cell culture conditions. These standard cell culture conditions preferably include a carbon dioxide humidified atmosphere comprised of 5 volume pecent of carbon dioxide and percent relative humidity.

In one embodiment, the signals monitored from the cultured cell samples are stochastic. As is known to those skilled in the art, the term “stochastic” refers to random variables. A stochastic signal is a sample function of a stochastic process. The process produces sample functions, the infinite collection of which is called the ensemble. Stochastic signals cannot be expressed exactly; they can be described only in terms of probabilities which may be calculated over the ensemble. Reference may be had, e.g., to pages 809-810 of Joseph D. Bronzino's “The Biomedical Engineerng Handbook,” CRC Press, Boca Raton, Fla., 1995.

Means for measuring stochastic signals are well known to those skilled in the art. Reference may be had, e.g., to U.S. Pat. No. 4,084,133 (method of and apparatus for determining the direction of the mutual temporal shift of at least two similar stochastic signals), U.S. Pat. No. 4,373,151 (stochastic demodulator for phase jump-modified signals), U.S. Pat. No. 5,703,906 (system for assessing stochastic properties of signals representing three different items of mutually orthogonal measurement information), U.S. Pat. No. 5,752,223 (code-excited linear predictive coder and decoder with conversion fitler for convertinig stochastic and impulsive excitation signals), U.S. Pat. No. 5,963,591 (system and method for stochastic characterization of a signal with four embedded orthogonal measurement data items), U.S. Pat. No. 6,008,642 (stochastic resonance detector for weak signals), U.S. Pat. No. 6,041,298 (method for synthesizing a frame of a speech signal with a computed stoachastic excitation part), U.S. Pat. No. 6,597,634 (system and method for stochastic characterization of sparase, four-dimensional, underwater sound signals), U.S. Pat. No. 6,724,188 (apparatus and method for measuring molecular electromagnetic signals with a squid device and stochastic resonance to measure low-threshhold signals.

In step 3314, a correlation is made between the “energetic signatures” of the healthy and unhealthy synchronized cells and the position they are in during the cell division cycle. Of particular interest is the “energetic signature of these cells” that occur prior to prophase.

In step 3316, the electronic signatures of the cells that occur prior to the prophases stage are analyzed to determine in what manner such electronic signatures may advantageously be modified.

In one embodiment, the electronic signatures are amplified, and such amplified signatures are returned to the biological system to facilitate the occurrence of cell division. In one aspect of this embodiment, the electronic signature(s) are amplified by a factor of about 1.5 to about 3.0; in this aspect, the amplified signatures may be used to facilitate cell division. In another aspect of this embodiment, the electronic signature(s) are amplified by a factor of at least 10; in this aspect, the substantially increased amplified signal confuses the cell and causes either its death and/or its return to interphase (G₀).

In one embodiment, the amplified electronic signature is at a power level of from about 0.1 to about 10 millwatts per square centimeter.

In step 3318, the cells are contacted with modified electronic signatures. In one embodiment, the modified electronic signatures effect, or tend to effect, selective cancellation of the “electronic signatures” of the cultured cells. In one aspect of this embodiment, the electronic signature(s) of the unhealthy cells are canceled by the cancellation signal, but the electronic signature(s) of the healthy cells are not so canceled by the cancellation signal(s).

Cancellation is the elimination of one quantity by another, as when a voltage is reduced to zero by antoher voltage of equal magnitude and opposite sign. Reference may be had, e.g. to U.S. Pat. No. 4,817,081 (adaptive filter for producing an echo cancellation signal in a transceiver system), U.S. Pat. No. 4,859,951 (detecting faults in transmission lines employing an echo cancellation signal), and the like. The entire disclosure of each of these United States patents is hereby incorporated by reference into this specification.

In one preferred embodiment, the cancellation signal effectuates phase cancellation by conventional means. Reference may be had, e.g., to U.S. Pat. No. 3,596,209 (sidelobe suprpression by phase cancellation in traveling wave devices), U.S. Pat. No. 4,233,626 (playback information record using phase cancellation for reading), U.S. Pat. No. 5,088,327 (phase cancellation enhancement of ultrasonic evlaution of metal-to-elastomer bonding), U.S. Pat. No. 5,898,454 (phase cancellation in a multi-output distribution amplifier at cross-over frequency), U.S. Pat. No. 5,913,172 (method and apparatus for reducing phase cancellation in a simulcast paging system), U.S. Pat. No. 6,700,442 (N-way phase cancellation power amplifier), and the like. The entire disclosure of each of these United States patents is hereby incorporated by reference into this specication.

In one embodiment, the modified electronic signatures effect, or tend to effect, selective interference with the electromagnetic signatures of the diseased cells. As is known to those skilled in the art, interference is the disturbing effect of any often undesired signal. Reference may be had, e.g., to U.S. Pat. No. 3,895,639, the entire disclosure of which is hereby incorporated by reference into this specification. This patent claims “1. Apparatus for producing an interference signal at a selected location comprising, in combination, oscillator means for furnishing an oscillator output signal having a determined frequency and a reference phase; phase shift means connected to said oscillator means cyclically varying the phase of said oscillator output signal, thereby furnishing a phase-shifted oscillator output signal; first electrode means connected to said oscillator means for creating a first current having said determined frequency at said selected location in response to said oscillator output signal; and second electrode means connected to said phase shift means for creating a second current having said determined frequency and a phase varying cyclically with respect to the phase of said first current at said selected location in response to said phase-shifted oscillator output signal, whereby interference between said first and second currents creates said interference signal at said selected location.” The entire disclosure of this United States patent is hereby incorporated by reference into this specification.

By way of yet further illustration, one may create an optically intererfering signal by the means disclosed in U.S. Pat. No. 3,695,749, the entire disclosure of which is hereby incorporated by reference into this specification. This patent claims “1. Apparatus for producing an interference pattern comprising means including a source of monochromatic coherent light for providing a pair of collimated coherent monochromatic light beams directed at an acute angle to each other toward a common point, and means for providing two closely spaced apparent light sources comprising a spreading lens positioned to intercept the light beams and having its primary focal point at the common point for spreading the beams in overlapping manner to provide a zone of light interference.”

In one preferred embodiment, the modified electronic signatures effectuate or tend to effectuate jamming of the electronic signatures of the diseased cells but not the healthy cells. As is known to those skilled in the art, jamming is the deliberate use of countermeasures, such as malicious transmission of interfering signals, to obstruct communication. One may effectuate jamming of the signals produced by the diseased cells by conventional means. Reference may be had, e.g., to U.S. Pat. No. 3,673,343 (anti-jamming circuit), U.S. Pat. No. 3,720,944 (signal system for jamming detection systems utilizing signal correlation), U.S. Pat. No. 4,122,452 (jamming signal cancellation system), U.S. Pat. No. 4,148,064 (jamming circuit for television signals), U.S. Pat. No. 4,214,208 (jamming of keyed continuous wave radio signals), U.S. Pat. No. 4,358,766 (jamming signal reduction system), U.S. Pat. No. 4,544,926 (adaptive jamming-signal canceler for radar receiver), U.S. Pat. No. 4,573,052 (method and device for reducing the power of jamming signals received by the sidelobes of a radar antenna), U.S. Pat. No. 4,651,204 (jamming signal generator circuit), U.S. Pat. No. 4,737,990 (unauthorized channel jamming signal appling method for CATV system), U.S. Pat. No. 4,748,667 (jamming singal scrambling and descrambling systems for CATV), U.S. Pat. No. 4,891,647 (method and device for reducing the power of jamming singals received by the secondary lobes of a random frequency radar antenna), U.S. Pat. No. 4,972,503 (method and apparatus for determining audience viewing habits by jamming a control signal and identifying the viewers command), U.S. Pat. No. 5,068,893 (television signal processing network for subscription televevision jamming signals), U.S. Pat. No. 5,228,082 (jamming singal producing system in CATV), U.S. Pat. No. 5,287,539 (interdiction program denial system for jamming audio and video signals), U.S. Pat. No. 5,363,104 (jamming signal cancellation system), U.S. Pat. No. 5,367,269 (system for producing an oscillating jamming signal using a phase-locked loop), U.S. Pat. No. 5,528,539 (interdiction program denial system for jamming audio and video signals), U.S. Pat. No. 5,793,795 (method for correcting errors from a jamming signal), U.S. Pat. No. 6,100,838 (multiple source jamming signal cnqacllation system), U.S. Pat. No. 6,757,324 (method and apparatus for detecting jamming signal), and the like. The entire disclosure of each of these United States patents is hereby incorporated by reference into this specification. The modified electronic signals are produced after first conducting.

FIG. 37 is a schematic illustration of an implantable assembly that is adapted to treat ill cells.

The cells are monitored for a sufficient period of time for them to go through each of the stages of cell division. These stages include phases of the cycle commonly referred to as G₀, G₁, G₂, and M phase, which includes prophase (during which the cells replicate their DNA and their centrosomes and begin the assembly of their mitotic spindle apparatuses), prometaphase (during which the cells dissolve their nuclear membranes and begin to align their chromosomes on the metaphase plate in preparation for the separation of sister chromatids), metaphase (at which time the chromosomes are aligned at the cell equator and the centrosomes, acting as the spindle pole bodies, are aligned on opposite sides of the cell, thereby defining its horizontal axis), anaphase (during which time the sister chromatids are synchronously separated towards opposite ends of the horizontal axis), telphoase (during which time the nuclear envelope of the two soon-to-be daughter cells begins assembly and the contractile ring responsible for cytoikinesis begins formation), and cytokinesis (during which the cell is physically divided by the shrinking contractile ring into the two resulting daughter cells). Reference may be had, e.g., to pages 1027-1061 of Bruce Alberts et al.'s “Molecular Biology of the Cell,” Fourth Editiion (Garland Science, New York, N.Y., 2002).

As is known to those skilled in the art, the process of cell division with mammalian cells generally takes at least 24 hours.

The invention has been described by reference to certain preferred embodiments. Various additions and modifications within the spirit of the invention will occur to those of skill in the art; and they are intended to be comprehended within the scope of the invention.

1. A biological polymer assembly comprised of a biological polymer, wherein said biological polymer is comprised of at least about 90 weight percent of tubulin, wherein said biological polymer is comprised of a positively charged segment, and wherein said positively charged segment has a molecular weight of at least about 5,000 Daltons, a bulk electrical conductivity of at least about 10⁻⁷ ohm⁻¹ meter⁻¹ Siemens, a concentration of elemental charges per cubic centimeter of from about 10¹² to about 10²⁵, and a length of at least about 2 nanometers.

2. The biological polymer assembly as recited in claim 1, wherein said biological polymer is comprised of at least about 90 weight percent of tubulin dimer.

3. The biological polymer assembly as recited in claim 1, wherein said positively charged segment has a bulk electrical conductivity of from about about 10⁻⁷ ohms⁻¹ meter⁻¹ Siemens to about 10⁻² ohm⁻¹ meter⁻¹ Siemens.

4. The biological polymer assembly as recited in claim 1, wherein said tubulin is comprised of alpha-tubulin.

5. The biological polymer assembly as recited in claim 1, wherein said tubulin is comprised of beta-tubulin.

6. The biological polymer assembly as recited in claim 1, wherein said positively charged segment has a molecular weight of at least 30,000 Daltons.

7. The biological polymer assembly as recited in claim 1, wherein said positively charged segment is comprised of at least 10¹⁴ positive charges per cubic centimeter.

8. The biological polymer assembly as recited in claim 1, wherein said tubulin is comprised of gamma-tubulin.

9. The biological polymer assembly as recited in claim 1, wherein said biological polymer is connected to a source of alternating current.

10. The biological polymer assembly as recited in claim 1, wherein a metal film is disposed on the surfaces of said biological polymer.

11. The biological polymer assembly as recited in claim 10, wherein said metal is selected from the group consisting of copper, gold, nickel, palladium, platinum, ruthenium, silver, and mixtures thereof.

12. The biological polymer assembly as recited in claim 11, wherein said biological polymer is a microtubule.

13. The biological polymer assembly as recited in claim 1, wherein said tubulin is comprised of tubulin dimer.

14. The biological polymer assembly as recited in claim 1, wherein said tubulin is comprised of tubulin oligomers.

15. The biological polymer assembly as recited in claim 1, wherein said tubulin is comprised of tubulin rings.

16. The biological polymer assembly as recited in claim 1, wherein said tubulin is comprised of tubulin spirals.

17. The biological polymer assembly as recited in claim 1, wherein said tubulin is comprised of tubulin ring crystals.

18. The biological polymer assembly as recited in claim 1, wherein said tubulin is comprised of tubulin ring fragments.

19. The biological polymer assembly as recited in claim 1, wherein said tubulin is comprised of tubulin hoops.

20. The biological polymer assembly as recited in claim 1, wherein said tubulin is comprised of tubulin C-ribbons.

21. The biological polymer assembly as recited in claim 1, wherein said tubulin is comprised of tubulin sheets.

22. The biological polymer assembly as recited in claim 1, wherein said tubulin is comprised of tubulin heaped sheets,

23. The biological polymer assembly as recited in claim 1, wherein said tubulin is comprised of tubulin S-ribbon.

24. The biological polymer assembly as recited in claim 1, wherein said tubulin is comprised of a microtubule associated protein-rich tubulin.

25. The biological polymer assembly as recited in claim 1, wherein said tubulin is derived from a naturally-occurring tubulin in which alanine has been substituted for serine.

26. The biological polymer assembly as recited in claim 1, wherein said positively charged segment has a molecular weight of at least about 10,000 Daltons.

27. The biological polymer assembly as recited in claim 1, wherein said positively charged segment has a molecular weight of at least about 15,000 Daltons.

28. The biological polymer assembly as recited in claim 1, wherein said positively charged segment has a molecular weight of at least about 30,000 Daltons.

29. The biological polymer assembly as recited in claim 1, wherein said positively charged segment has a molecular weight of at least about 40,000 Daltons.

30. The biological polymer assembly as recited in claim 1, wherein said positively charged segment has a molecular weight of at least about 1,000,000 Daltons.

31. The biological polymer assembly as recited in claim 1, wherein said positively charged segment has a bulk electrical conductivity of from about 10⁻⁷ ohm⁻¹ meter⁻¹ Siemens to about 10⁸ ohm⁻¹ meter⁻¹ Siemens.

32. The biological polymer assembly as recited in claim 1, wherein said positively charged segment has a bulk electrical conductivity of from about 10⁻⁷ ohm⁻¹ meter⁻¹ Siemens to about 10⁻² ohm⁻¹ meter⁻¹ Siemens.

33. The biological polymer assembly as recited in claim 1, wherein said positively charged segment is comprised of at least about 10¹⁴ elemental charges per cubic centimeter.

34. The biological polymer assembly as recited in claim 1, wherein said positively charged segment is comprised of at least about 10¹⁷ elemental charges per cubic centimeter.

35. The biological polymer assembly as recited in claim 1, wherein said positively charged segment is comprised of at least about 10¹⁸ elemental charges per cubic centimeter.

36. The biological polymer assembly as recited in claim 1, wherein said positively charged segment is comprised of at least about 10¹⁹ elemental charges per cubic centimeter.

37. The biological polymer assembly as recited in claim 1, wherein said positively charged segment is comprised of at least about 10²⁰ elemental charges per cubic centimeter.

38. The biological polymer assembly as recited in claim 1, wherein said elemental charges have a drift mobility of at least about 10 square centimeters/volt/second.

39. The biological polymer assembly as recited in claim 1, wherein said elemental charges have a drift mobility of at least about 50 square centimeters/volt/second.

40. The biological polymer assembly as recited in claim 1, wherein said elemental charges have a drift mobility of at least about 100 square centimeters/volt/second.

41. The biological polymer assembly as recited in claim 1, wherein said elemental charges have a drift mobility of at least about 1,000 square centimeters/volt/second.

42. The biological polymer assembly as recited in claim 1, wherein said elemental charges have a drift mobility of at least about 5,000 square centimeters/volt/second.

43. The biological polymer assembly as recited in claim 1, wherein said biological polymer is comprised of a negatively charged segment, and wherein said negatively charged segment has a molecular weight of at least about 5,000 Daltons, a bulk electrical conductivity of at least about 10⁻⁷ ohm⁻¹ meter⁻¹ Siemens, a concentration of elemental charges per cubic centimeter of from about 10 to about 10²⁵, and a length of at least about 2 nanometers.

44. The biological polymer assembly as recited in claim 43, wherein said positively charged segment and said negatively charged segment are contiguous with each other.

45. The biological polymer assembly as recited in claim 1, wherein said assembly further comprises a conductive oligonucleotide link attached to said biological polymer.

46. The biological polymer assembly as recited in claim 45, wherein said conductive oligonucleotide link is comprised of conductive deoxyribonucleic acid in which the imino proton of each base pair has been substituted with a metal ion.
